(19) United States
(12) Patent Application Publication
(10) Pub. No.: US 2024/0173436 A1
(43) Pub. Date: May 30, 2024

(54) COMPACT PROMOTERS FOR GENE EXPRESSION

(71) Applicant: HUNTERIAN MEDICINE LLC, Cambridge, MA (US)

(72) Inventor: Vinod Jaskula-Ranga, Cambridge, MA (US)

(21) Appl. No.: 18/285,369

(22) PCT Filed: Mar. 31, 2022
(86) PCT No.: PCT/US22/22920

§ 371 Date: Oct. 2, 2023

Related U.S. Application Data
(60) Provisional application No. 63/168,708, filed on Mar. 31, 2021.

(51) Int. Cl. A61K 48/00; A61P 1/00; A61P 11/00; C12N 15/86

(52) U.S. Cl. CPC A61K 48/0066; A61P 1/00; A61P 11/00; C12N 15/86; C12N 2750/14143; C12N 2800/22; C12N 2830/15; C12N 2830/42

(57) ABSTRACT

The invention relates generally to compact promoters and their use in expressing genes, e.g., for treating disease.

### CROSS-REFERENCE TO RELATED APPLICATIONS

This application claims the benefit of and priority to U.S. Provisional Application No. 63/168,708, filed Mar. 31, 2021, the entire contents of which are incorporated by reference herein.

### FIELD OF THE INVENTION

The invention relates generally to compact promoters and their use in expressing genes, e.g., for treating disease.

### BACKGROUND

Adeno-associated viruses (AAV) provide a safe means of therapeutic gene delivery; however, a significant technical obstacle limits an AAV vector's utility: its small payload capacity. The large size of certain genes, including for example, the Cystic Fibrosis Transmembrane Conductance Regulator (CFTR) gene, in addition to a promoter, terminator, and 2 inverted terminal repeats (ITRs), presents a significant barrier to AAV packaging. For example, the full length CFTR gene, including its promoter, terminator, and 2 inverted terminal repeats (ITRs), has been viewed as too big to fit into a single AAV vector, making gene delivery impossible. Efforts at gene therapy for CF have nonetheless been pursued for several decades, since identification and cloning of the CFTR gene. Initial efforts were aimed at fitting the expression cassette within a single AAV by eliminating the promoter entirely. Although these pioneering studies advanced to clinical trials, CFTR expression and functional rescue were not observed. More recent attempts at overcoming the limited payload capacity of AAV were focused on a combination of small synthetic promoters and a truncated CFTR gene.

Other large genes, such as the ATP7B gene which is mutated in Wilson's disease, the ATP7A gene which is mutated in Menkes disease, the AGL gene which is mutated in Cori Disease, the dystrophin gene which is mutated in Duchenne muscular dystrophy (DMD), and the CPS1 gene which is mutated in carbamoyl phosphate synthetase I deficiency (CPS1D), face similar barriers to AAV packaging.

Accordingly, there is a need in the art for compositions and methods for packaging large genes in vectors such as AAV, which are suitable for gene delivery.

### SUMMARY OF THE INVENTION

The invention is based, in part, upon the discovery that compact promoters can effectively drive expression of large genes useful in, for example, gene therapy applications. AAV represents a promising delivery vehicle for nucleic acids for gene therapy, but the small size of AAV is a barrier to delivery of large genes, such as those having coding sequences above about 4000 bp, and vector components. Here, the disclosure provides a solution to this problem using a compact promoter to deliver sufficient and sustained expression of genes, e.g., large genes such as CFTR, via AAV.

The invention is also based, in part, upon the discovery that CFTR sequences can be optimized based on an iterative RNA-folding and codon optimization process, generating sequences representing a range of thermodynamic stability. Such codon-optimized CFTR sequences enhance CFTR expression, processing, and function.

Accordingly, in one aspect, the disclosure relates to a nucleic acid including a compact promoter operably linked to a coding sequence of a gene, wherein the compact promoter is between 50 and 250 bp, and wherein the coding sequence of the gene is greater than about 4000 bp. In certain embodiments, the compact promoter is between 75 and 225 bp. In certain embodiments, the compact promoter is between 100 and 200 bp. In certain embodiments, the compact promoter is between 150 and 180 bp.

In certain embodiments, the promoter includes a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3), or a functional fragment thereof, or a variant having a nucleic acid sequence at least about 80%, at least about 85%, at least about 90%, at least about 95%, at least about 98%, at least about 99%, or at least about 99.5% identity thereto.

In certain embodiments, the compact promoter includes an H1 promoter. In certain embodiments, the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a functional fragment thereof, or a variant having a nucleic acid sequence at least about 80%, at least about 85%, at least about 90%, at least about 95%, at least about 98%, at least about 99%, or at least about 99.5% identity thereto. In certain embodiments, the H1 promoter includes a human H1 promoter.

In certain embodiments, the compact promoter includes a Gar1 promoter. In certain embodiments, the Gar1 promoter is selected from SEQ ID NOs: 107-203 or a functional fragment thereof, or a variant having a nucleic acid sequence having at least about 80%, at least about 85%, at least about 90%, at least about 95%, at least about 98%, at least about 99%, or at least about 99.5% identity thereto. In certain embodiments, the Gar1 promoter is a human Gar1 promoter.

In certain embodiments, the compact promoter includes a bidirectional promoter selected from SEQ ID NOs: 204-255 or a functional fragment thereof, or a variant having a nucleic acid sequence at least about 80%, at least about 85%, at least about 90%, at least about 95%, at least about 98%, at least about 99%, or at least about 99.5% identity thereto.

In certain embodiments, the compact promoter does not comprise a viral promoter and/or a synthetic promoter. In certain embodiments, the compact promoter does not comprise F5tg83.

In certain embodiments, the compact promoter includes at least 95%, 98%, 99%, 99.5% or 100% identity to a naturally-occurring mammalian promoter.

In certain embodiments, compact promoter is capable of expressing a luciferase reporter at a higher level than is a HSK thymidine kinase (TK) promoter.

In certain embodiments, the coding sequence encodes a cystic fibrosis transmembrane conductance regulator (CFTR), ATP7B, ATP7A, AGL, DMD, CPS1, or a functional fragment or variant thereof.

In certain embodiments, the coding sequence encodes a cystic fibrosis transmembrane conductance regulator (CFTR). In certain embodiments, the CFTR coding sequence is codon optimized.

In certain embodiments, the codon-optimized CFTR coding sequence includes one or more of the following features as compared to a wild type CFTR coding sequence: (a) fewer unpaired base pairs of mRNA; (b) increased codon usage bias; (c) decreased GC content; (d) fewer CpG dinucleotides; (e) increased mRNA secondary structure; (f) fewer cryptic splicing sites; (g) fewer premature poly(A) sites; (h) fewer RNA instability motifs; (i) fewer AT-rich elements (ARE); (j) fewer repeat sequences (e.g., direct repeat, reverse repeat, and dyad repeat); (k) fewer GC peaks; and (l) fewer cis-acting elements.

In certain embodiments, the CFTR coding sequence includes a truncated form of a wild-type CFTR gene. In certain embodiments, the truncated form of the wild-type CFTR gene includes CTFRΔR.

In another aspect, the disclosure relates to an expression construct including a nucleic acid as described herein. In certain embodiments, the coding sequence can be expressed in the lung, pancreas, liver, neuron, or combinations thereof. In certain embodiments, the expression construct is expressed in HEK293, A549 cells, CFBE4lo−, A549, or Calu-3 cells.

In certain embodiments, the coding sequence encodes a CFTR, and, when the expression construct is expressed in an epithelial cell, the expressed CFTR protein causes an increase in transepithelial electrical resistance as compared to a cell in which the expression construct is not present. In certain embodiments, the coding sequence encodes a CFTR, and, when the expression construct is expressed in an epithelial cell, the expressed CFTR protein causes an increase in transepithelial Cl⁻ transport as compared to a cell in which the expression construct is not present.

In another aspect, the disclosure relates to a vector having an expression construct as described herein.

In certain embodiments, the vector includes an adeno-associated viral (AAV) vector. In certain embodiments, the AAV vector includes an AAV-6 vector.

In another aspect, the disclosure relates to a method of expressing a protein in a cell, the method including transfecting a cell with an expression construct as described herein or a vector as described herein.

In another aspect, the disclosure relates to a method of treating a disease (e.g., cystic fibrosis, Wilson disease, Menkes disease, Cori Disease, Duchenne Muscular Dystrophy, or carbamoyl phosphate synthetase I deficiency (CPS1D)) in a subject in need thereof, the method including administering to the subject a vector as described herein.

In another aspect, the disclosure relates to a nucleic acid having a cystic fibrosis transmembrane conductance regulator (CFTR) coding sequence, wherein the CFTR coding sequence is codon optimized, wherein the CFTR coding sequence includes one or more of the following features as compared to a wild type CFTR coding sequence: (a) fewer unpaired base pairs of mRNA; (b) increased codon usage bias; (c) decreased GC content; (d) fewer CpG dinucleotides; (e) increased mRNA secondary structure; (f) fewer cryptic splicing sites; (g) fewer premature poly(A) sites; (h) fewer RNA instability motifs; (i) fewer AT-rich elements (ARE); (j) fewer repeat sequences (e.g., direct repeat, reverse repeat, and dyad repeat); (k) fewer GC peaks; and (l) fewer cis-acting elements.

In certain embodiments, the codon usage bias is determined using the codon adaptive index (CAI). In certain embodiments, the CAI score is greater than about 0.70.

In certain embodiments, the frequency of optimal codons (FOP) is greater than about 80%.

In certain embodiments, the cis-acting element is selected from the group consisting of splice donors/acceptors (e.g., GGTAAG, GGTGAT, GTAAAA, GTAAGT), PolyA (e.g., AATAAA, ATTAAA, AAAAAAA), destabilizing motifs (e.g., ATTTA), AT-rich elements (e.g., ATTTTA, ATTTTTA, ATTTTTTA), PolyT, polymerase slippage sites (e.g., GGGGGG, CCCCCC), and internal Kozak sequences (e.g., ACCACCATGG, GCCACCATGG).

In certain embodiments, the nucleic acid further includes a 3′UTR, a 5′UTR or a 3′UTR and a 5′UTR. In certain embodiments, the minimum free energy structure of the nucleic acid having the 3′UTR, the 5′UTR or the 3′UTR and the 5′UTR does not favor base-pairing between (a) the 3′UTR, the 5′UTR or the 3′UTR and the 5′UTR and (b) the CFTR coding sequence.

In another aspect, the disclosure relates to an expression construct comprising a codon-optimized CFTR coding sequence as described herein. In certain embodiments, the half-life of the mRNA expressed from the codon optimized CFTR coding sequence is increased as compared to a wild-type CFTR coding sequence. In certain embodiments, expression of the codon optimized CFTR coding sequence results in an increased amount of CFTR mRNA or protein as compared to expression of a wild-type CFTR coding sequence. In certain embodiments, the CFTR coding sequence can be expressed in the lung and/or the pancreas. In certain embodiments, the expression construct can be expressed in HEK293 or A549 cells.

In certain embodiments, when the expression construct is expressed in an epithelial cell, the expressed CFTR protein causes an increase in transepithelial electrical resistance as compared to a cell in which the expression construct is not present. In certain embodiments, when the expression construct is expressed in an epithelial cell, the expressed CFTR protein causes an increase in transepithelial Cl⁻ transport as compared to a cell in which the expression construct is not present.

In another aspect, the disclosure relates to a vector having an expression construct including a codon-optimized CFTR coding sequence as described herein.

In certain embodiments, the vector includes an adeno-associated viral (AAV) vector. In certain embodiments, the AAV vector includes an AAV-6 vector.

In another aspect, the disclosure relates to a method of expressing a CFTR protein in a cell, the method including transfecting a cell with an expression construct or vector as described herein.

In another aspect, the disclosure relates to a method of treating cystic fibrosis in a subject in need thereof, the method including administering to the subject a vector as described herein.

In another aspect, the disclosure relates to a nucleic acid including a compact bidirectional promoter, a protein coding gene, and a second gene. In some embodiments, the compact bidirectional promoter has a size between 50 and 250 bp, has at least one regulatory element that provides for transcription of the protein coding gene in one direction and at least one regulatory element that provides for transcription of the second gene in the other direction, wherein the protein coding gene includes a coding sequence from about 300 bp to about 4110 bp. These and other aspects and features of the invention are described in the following detailed description and claims.

### DESCRIPTION OF THE DRAWINGS

The invention can be more completely understood with reference to the following drawings.

FIG. 1 is a schematic showing the region in which the H1 promoter is located, between the start of the H1RNA gene (left) to the start of the PARP-2 gene (right). Transcription factor binding sites including Staf, DSE, PSE, c-REL, GATA-1, GATA-2, and CREB are shown. In addition, the B recognition sequence (BRE) and TATA box are shown.

FIG. 2 provides Hidden Markov model (HMM) used to identify H1 promoter sequences.

FIG. 3 provides an alignment of Artiodactyla, Camivora, Cetacea, Chiroptera, Insectivore, Lagomorpha, Marsupial, Pangolin, Perissodactyla, Primate, Rodent, and Xenartha H1 promoters.

FIG. 4 provides an alignment of human and Orycteropus afer H1 promoters, showing the 132 bp insertion and 12 bp insertion found in the Orycteropus afer H1 promoter. The human H1 promoter corresponds to SEQ ID NO: 87 and the Orycteropus afer H1 promoter corresponds to SEQ ID NO: 25. The consensus sequence corresponds to SEQ ID NO: 1808.

FIG. 5 provides an alignment of H1 promoter sequences from Artioactyla species.

FIG. 6 provides an alignment of H1 promoter sequences from Carnivora species.

FIG. 7 provides an alignment of H1 promoter sequences from Cetacea species.

FIG. 8 provides an alignment of H1 promoter sequences from Chiroptera species.

FIG. 9 provides an alignment of H1 promoter sequences from Dermoptera species.

FIG. 10 provides an alignment of H1 promoter sequences from Hyracoidae species.

FIG. 11 provides an alignment of H1 promoter sequences from Insectivora species.

FIG. 12 provides an alignment of H1 promoter sequences from Lagomorpha species.

FIG. 13 provides an alignment of H1 promoter sequences from Marsupial species.

FIG. 14 provides an alignment of H1 promoter sequences from Pangolin species.

FIG. 15 provides an alignment of H1 promoter sequences from Perissodactyla species.

FIG. 16 provides an alignment of H1 promoter sequences from Primate species.

FIG. 17 provides an second alignment of H1 promoter sequences from Primate species showing the TATA box, PSE, Staf, and DSE binding sites.

FIG. 18 provides an alignment of H1 promoter sequences from Rodent species.

FIG. 19 provides an alignment of H1 promoter sequences from Xenartha species.

FIG. 20 depicts RT-qPCR design for CFTR coding sequence variants. By using a common 3′UTR region, priming and probe detection is consistent for all variants. RT priming with oligo(dT) may reduce problems with mRNA synthesis due to extensive secondary structure.

FIG. 21A depicts DNA alignment and conservation of the H1 bidirectional promoter, from the start of the H1RNA gene (left) to the start of the PARP-2 gene (right). FIG. 21B depicts RNA polymerase II-driven promoter activity in HeLa cells. Also depicted is the length of each promoter shown in the red bars, plotted against the right Y axis.

FIG. 22 depicts CFTR mRNA secondary structure. The secondary structure for the WT CFTR mRNA is shown (left with the calculated free energy shown below the structure. The secondary structure for a candidate optimized structure is shown (right) with the calculated free energy show below the structure. Extensive base-paring through the designed mRNA is seen. The calculated CAI for the structure on the right is greater than for the structure on the left.

FIG. 23 provides a schematic representation of mouse H1 promoter deletion constructs evaluated as described in Example 5.

FIG. 24 shows an alignment of mouse H1 promoter deletion constructs evaluated as described in Example 5.

FIG. 25 shows a bar graph showing normalized firefly to NANOLUC® luciferase signal for each mouse H1 promoter deletion constructs described in Example 5.

FIG. 26 provides a schematic representation of 17 mouse H1 promoter mutation constructs that were designed by walking across the promoter in 10 bp increments and replacing the sequence with its reverse complement.

FIG. 27 provides a sequence alignment of the mouse H1 promoter mutation constructs provided in FIG. 26.

FIG. 28 shows a bar graph showing normalized firefly to NANOLUC® luciferase signal for each mouse H1 promoter mutation constructs described in Example 6.

FIG. 29 provides a schematic representation of 12 constructs designed to incorporate introns into the mouse H1 promoter region.

FIG. 30 shows a bar graph showing normalized firefly to NANOLUC® luciferase signal for each mouse H1 intron constructs described in Example 7.

FIG. 31 provides a schematic showing the design of human H1 promoter and variant constructs. As shown in FIG. 31, a construct carrying a human H1 promoter alone (p144), a human H1 promoter with a 9 bp Kozak sequence (GCCGCCACC) (SEQ ID NO: 256) (p145), a human H1 promoter with a beta-globin 5′UTR (p146), and a human H1 promoter with a TATA box mutation (TATAA->TCGAA) (p147) were designed.

FIG. 32 provides a sequence alignment of the constructs provided in FIG. 31.

FIG. 33 shows a bar graph showing normalized firefly to NANOLUC® luciferase signal for each human H1 wt and 5′UTR construct described in Example 8.

FIG. 34 provides a schematic showing the design of mouse H1 promoter and 5′UTR variant constructs.

FIG. 35 provides a sequence alignment of the constructs provided in FIG. 34.

FIG. 36 shows a bar graph showing normalized firefly to NANOLUC® luciferase signal for each mouse H1 wt and 5′UTR construct described in Example 8.

FIG. 37 shows a bar graph showing normalized firefly to NANOLUC® luciferase signal for each bidirectional promoter construct described in Example 9. The promoters were human H1 (p144; SEQ ID NO: 87), mouse H1 (p148; SEQ ID NO: 93), human 7sk-1 (p199; SEQ ID NO: 242), mouse 7sk-1 (p203; SEQ ID NO: 204), human ALOXE3 (p204; SEQ ID NO: 246), human CGB1 (p206; SEQ ID NO: 247), human CGB2 (p207; SEQ ID NO: 248), human GAR1-1 (p216; SEQ ID NO: 107), human Med16-1 (p222; SEQ ID NO: 249), human Med16-2 (p223; SEQ ID NO: 250), human SRP (p242; SEQ ID NO: 233).

FIG. 38 is a graph showing the optimization of a luciferase reporter assay. HEK293 cells were co-transfected with firefly luciferase and NANOLUC® reporter plasmids under the control of standard promoters p006 (EF1a), p323 (PGK), and p322 (TK). Normalized luciferase expression (firefly:NANOLUC®) was quantified for transfection ratios of 90:10 ng, 99:1 ng, and 100:0.1 ng.

FIG. 39 is a bar graph showing normalized luciferase signal (firefly: NANOLUC®) for a library of H1 promoters including p095, p127, p110, p109, p088, p094, p060, p071, p077, p103, p10⁰, p102, p092, p073, p100, p102, p092, p073, p083, p130, p066, p089, p112, p101, p099, p116, p098, p069, p106, p131, p081, p107, p074, p072, p082, p097, p108, p065, p122, p114, p070, p091, p062, p119, p113, p063, p064, p090, p079, p105, p067, p128, p124, p084, p126, p078, p086, p093, p059, p058, p087, p061, p085, p129, p096, p111, p125, p115, p068, p118, p117, p076, p120, p123, and p104 in CFBE4lo− cells. Control TK promoter normalized luciferase activity is shown as “p322”.

FIG. 40 is a bar graph showing normalized luciferase signal (firefly: NANOLUC®) for a library of H1 promoters including p095, p127, p088, p094, p087, p110, p109, p083, p100, p073, p116, p092, p077, p066, p130, p101, p079, p071, p081, p119, p065, p098, p097, p060, p061, p089, p078, p070, p102, p084, p086, p059, p099, p106, p069, p125, p117, p058, p067, p129, p126, p107, p122, p064, p112, p062, p085, p091, p082, p072, p131, p090, p093, p063, p068, p114, p120, p115, p074, p076, p108, p113, p096, p124, p105, p103, p118, p128, p111, p123, and p104 in A549 cells. Control TK promoter normalized luciferase activity is shown as “p322”.

FIG. 41 is a bar graph showing normalized luciferase signal (firefly: NANOLUC®) for a library of H1 promoters including p095, p127, p094, p110, p107, p109, p102, p084, p071, p087, p101, p088, p097, p092, p066, p077, p106, p065, p099, p078, p116, p081, p119, p083, p098, p131, p073, p112, p100, p062, p103, p091, p061, p072, p129, p068, p114, p120, p060, p070, p118, p059, p113, p089, p108, p069, p067, p122, p124, p058, p079, p115, p093, p130, p086, p074, p125, p063, p126, p117, p090, p076, p096, p128, p105, p111, p123, p085, p082, p064, and p104 in Calu3 cells. Control TK promoter normalized luciferase activity is shown as “p322”.

FIG. 42A is a violin plot showing log-scale expression of a library of H1 promoters in three lung cell types (CFBE4lo−, A549, and Calu3). Vertical axis represents relative luminescence units.

FIG. 42B is a violin plot showing log-scale expression of a library of H1 promoters in Calu-3 cells compared to the expression activity of standard promoters TK, PGK, and EF1a.

FIG. 43 is a series of graphs showing linear regression analysis to compare the expression activity of each of the promoters in the library (each dot represents a promoter) in different cell types.

FIG. 44 is a plot showing hierarchical clustering of a library of H1 promoters segregated by activity in three lung cell types (CFBE4lo− marked with a *, A549 marked with a †, and Calu3 marked with a ‡) and one control cell type (HeLa marked with a ♦).

### DETAILED DESCRIPTION

Various features and aspects of the invention are discussed in more detail below.

The invention is based, in part, upon the discovery that compact promoters can effectively drive expression of large genes useful in, for example, gene therapy applications. AAV represents a promising delivery vehicle for nucleic acids for gene therapy, but the small size of AAV is a barrier to delivery of large genes, such as those having coding sequences above about 4000 bp, and vector components. Here, the disclosure provides a solution to this problem using a compact promoter to deliver sufficient and sustained expression of genes, e.g., large genes such as CFTR, via AAV.

The invention is also based, in part, upon the discovery that CFTR sequences can be optimized based on an iterative RNA-folding and codon optimization process, generating sequences representing a range of thermodynamic stability. Such codon-optimized CFTR sequences enhance CFTR expression, processing, and function.

Accordingly, the disclosure provides nucleic acids, expression constructs, and vectors comprising a compact promoter and a gene, e.g., a gene having more than about 4000 bp, wherein the compact promoter is small enough to allow for the inclusion of a large gene in a vector, such as an AAV vector, having a size limit that makes expression of large genes difficult using conventional promoters. Unless otherwise defined herein, scientific and technical terms used in this application shall have the meanings that are commonly understood by those of ordinary skill in the art.

Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Exemplary methods and materials are described herein, although methods and materials similar or equivalent to those described herein can also be used in the practice or testing of the present disclosure. The materials, methods, and examples are illustrative only and not intended to be limiting.

### I. Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

IUPAC nucleotide code is used throughout. IUPAC nucleotide code is provided in TABLE 1.

TABLE 1

| | |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | gap |

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the term “functional fragment” refers to a fragment of (a) a promoter or (b) a gene or coding sequence (e.g., an mRNA) that encodes a protein that retains, for example, at least 10%, at least 20%, at least 30%, at least 40%, at least 50%, at least 60%, at least 70%, at least 80%, at least 90%, at least 95%, or 100% of at least one activity of the corresponding full-length, naturally occurring promoter or protein.

As used herein, the term “variant” refers to a variant of (a) a promoter or (b) a gene or coding sequence (e.g., an mRNA) that encodes a protein that retains, for example, at least 10%, at least 20%, at least 30%, at least 40%, at least 50%, at least 60%, at least 70%, at least 80%, at least 90%, at least 95%, or 100% of at least one activity of the corresponding full-length, naturally occurring promoter or protein. For example, a variant can comprise a splice variant or a gene comprising a mutation such as an insertion, deletion, or substitution.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. The term host cell may refer to the packaging cell line in which the rAAV is produced from the plasmid. In the alternative, the term “host cell” may refer to the target cell in which expression of the transgene is desired.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector based on an adeno-associated virus comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. An rAAV vector can be packaged into an AAV virus capsid to generate a “recombinant adeno-associated viral particle (rAAV particle)”.

An “rAAV virus” or “rAAV viral particle” refers to a viral particle composed of at least one AAV capsid protein and an encapsidated rAAV vector genome.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR.

A “helper virus” for AAV refers to a virus that allows AAV (which is a defective parvovirus) to be replicated and packaged by a host cell. A number of such helper viruses are known in the art.

As used herein, “expression control sequence” means a nucleic acid sequence that directs transcription of a nucleic acid. An expression control sequence can be a promoter, such as a constitutive promoter, or an enhancer. The expression control sequence is operably linked to the nucleic acid sequence to be transcribed.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

As used herein, the terms “prevent,” “preventing” and “prevention” refer to the prevention of the recurrence or onset of, or a reduction in one or more symptoms of a disease or condition in a subject as result of the administration of a therapy (e.g., a prophylactic or therapeutic agent). For example, in the context of the administration of a therapy to a subject for an infection, “prevent,” “preventing” and “prevention” refer to the inhibition or a reduction in the development or onset of a disease or condition, or the prevention of the recurrence, onset, or development of one or more symptoms of a disease or condition, in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition, treatment refers to the reduction or amelioration of the progression, severity, and/or duration of one or more symptoms of the disease, or the amelioration of one or more symptoms resulting from the administration of one or more therapies (including, but not limited to, the administration of one or more prophylactic or therapeutic agents).

“Administering” or “administration of a substance, a compound or an agent to a subject can be carried out using one of a variety of methods known to those skilled in the art. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Each embodiment described herein may be used individually or in combination with any other embodiment described herein.

### II. Compact Promoters

The disclosure is based, in part, upon the discovery that compact promoters can effectively drive expression of large genes useful in, for example, gene therapy applications such as those involving AAV. The size limitations of AAV make it difficult to use for the expression of large genes, such as those having coding sequences above about 4110 bp. However, this problem can be overcome by using a compact promoter, as described herein, to deliver sufficient and sustained expression of genes, e.g., large genes such as CFTR, via AAV.

A compact promoter provided herein can be selected to express the selected transgene in a desired target cell. In some embodiments, the target cell is a lung cell, a pancreatic cell, a liver cell, or a neuronal cell. The promoter may be derived from any species, including human. In one embodiment, the promoter is “cell specific.” The term “cell-specific” means that the particular promoter selected for the recombinant vector can direct expression of the selected transgene in a particular cell.

In certain embodiments, the promoter is of a small size, e.g., less than about 500 bp, due to the size limitations of the AAV vector. In certain embodiments, the promoter is less than about 300 bp, less than about 200 bp, between about 50 bp and about 400 bp, between about 75 bp and about 400 bp, between about 99 bp and about 400 bp, between about 100 bp and about 400 bp, between about 150 bp and about 400 bp, between about between about 200 bp and about 400 bp, between about 250 bp and about 400 bp, between about 300 bp and about 400 bp, about 50 bp and about 300 bp, about 75 bp and about 300 bp, about 100 bp and about 300 bp, about 150 bp and about 300 bp, between about 200 bp and about 300 bp, about 50 bp and about 250 bp, about 75 bp and about 250 bp, between about 100 bp and about 250 bp, between about 150 bp and about 250 bp, between about 200 bp and about 250 bp, between about 50 bp and about 200 bp, between about 75 bp and about 200 bp, between about 100 bp and about 200 bp, between about 150 bp and about 200 bp, between about 50 bp and about 150 bp, between about 100 bp and about 150 bp, between about 50 bp and about 150 bp, and between about 100 bp and about 150 bp in size.

In certain embodiments, the promoter is a bidirectional promoter. In certain embodiments, the bidirectional promoter is less than about 500 bp. In certain embodiments, the bidirectional promoter is less than about 300 bp, less than about 200 bp, between about 50 bp and about 400 bp, between about 75 bp and about 400 bp, between about 99 bp and about 400 bp, between about 100 bp and about 400 bp, between about 150 bp and about 400 bp, between about between about 200 bp and about 400 bp, between about 250 bp and about 400 bp, between about 300 bp and about 400 bp, between about 50 bp and about 300 bp, between about 75 bp and about 300 bp, between about 100 bp and about 300 bp, between about 150 bp and about 300 bp, between about 200 bp and about 300 bp, between about 50 bp and about 250 bp, between about 75 bp and about 250 bp, between about 100 bp and about 250 bp, between about 150 bp and about 250 bp, between about 200 bp and about 250 bp, between about 50 bp and about 200 bp, between about 75 bp and about 200 bp, between about 100 bp and about 200 bp, between about 150 bp and about 200 bp, between about 50 bp and about 150 bp, between about 100 bp and about 150 bp, between about 50 bp and about 150 bp, and between about 100 bp and about 150 bp in size.

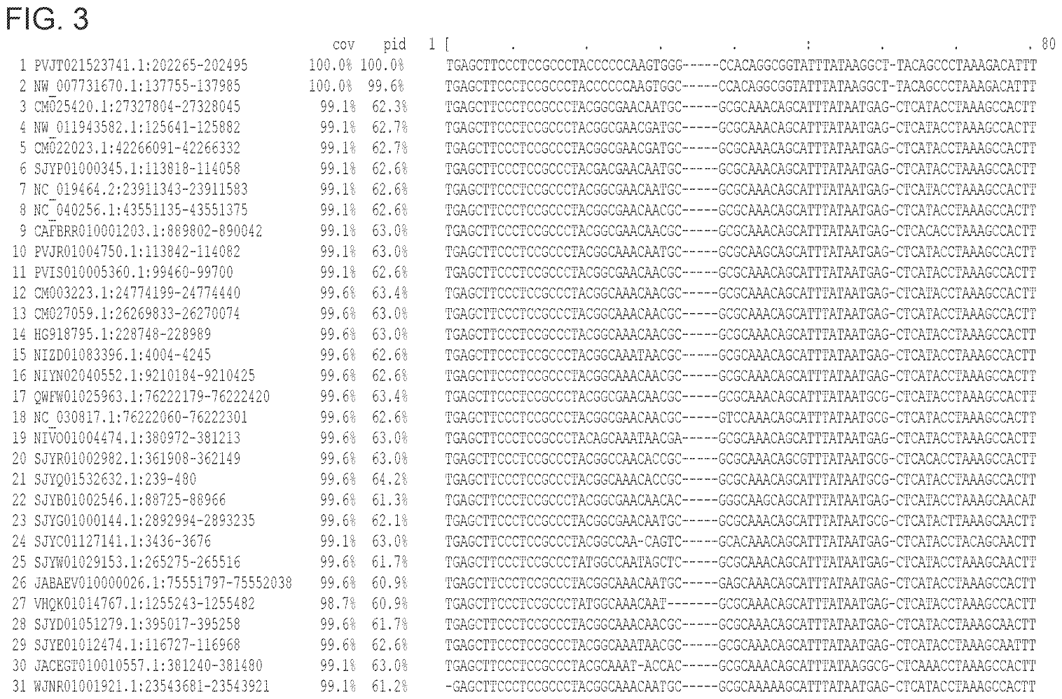

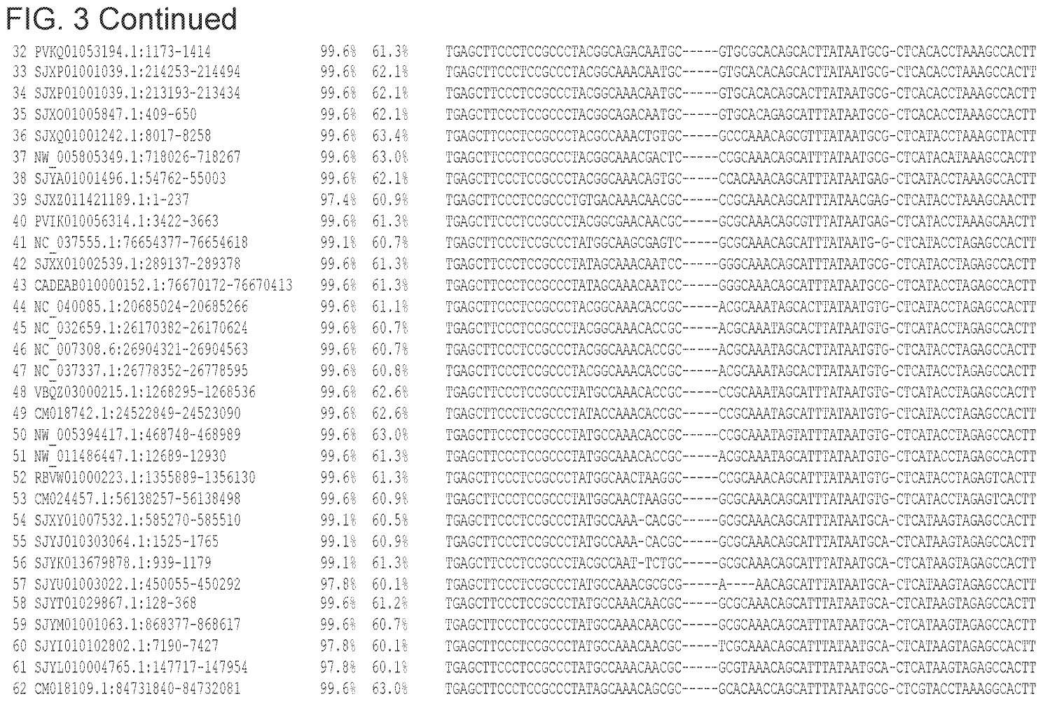

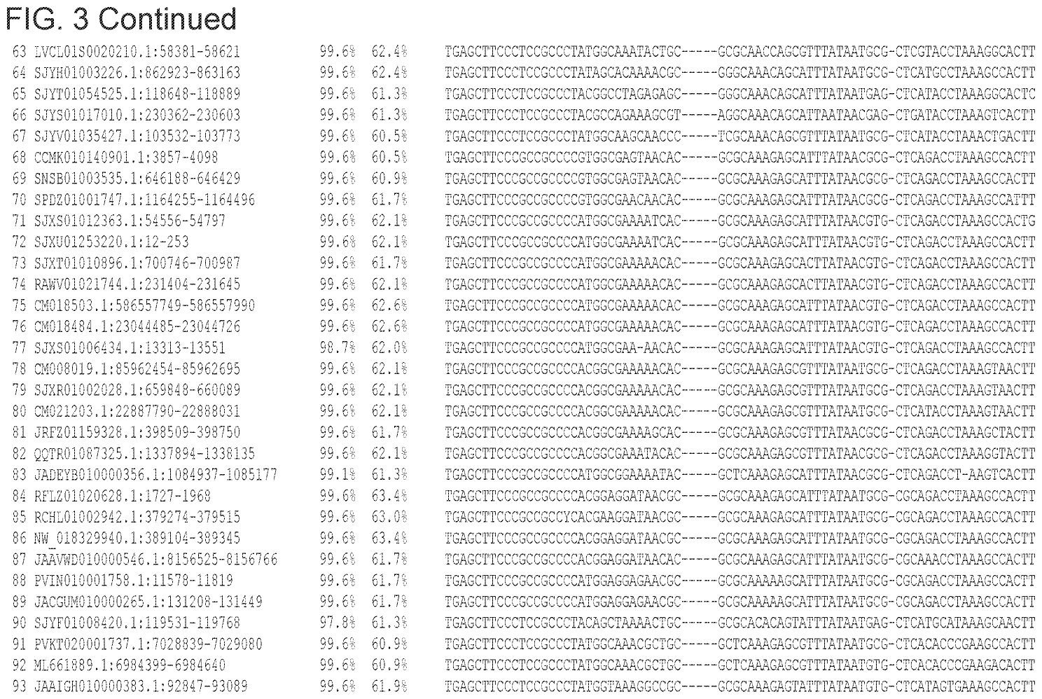

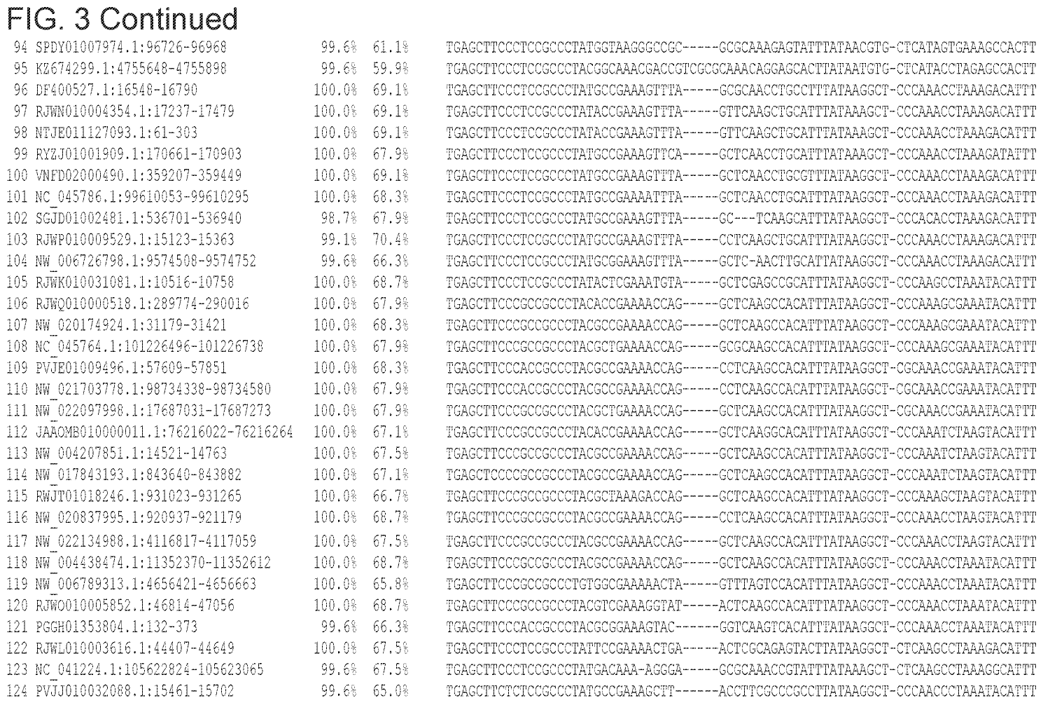

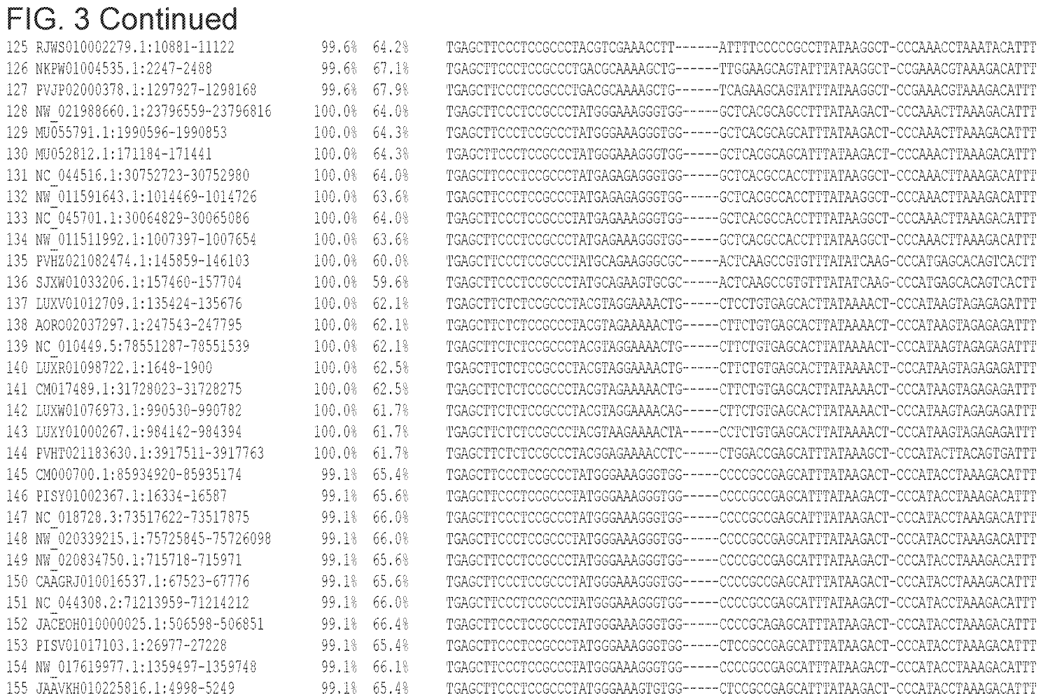

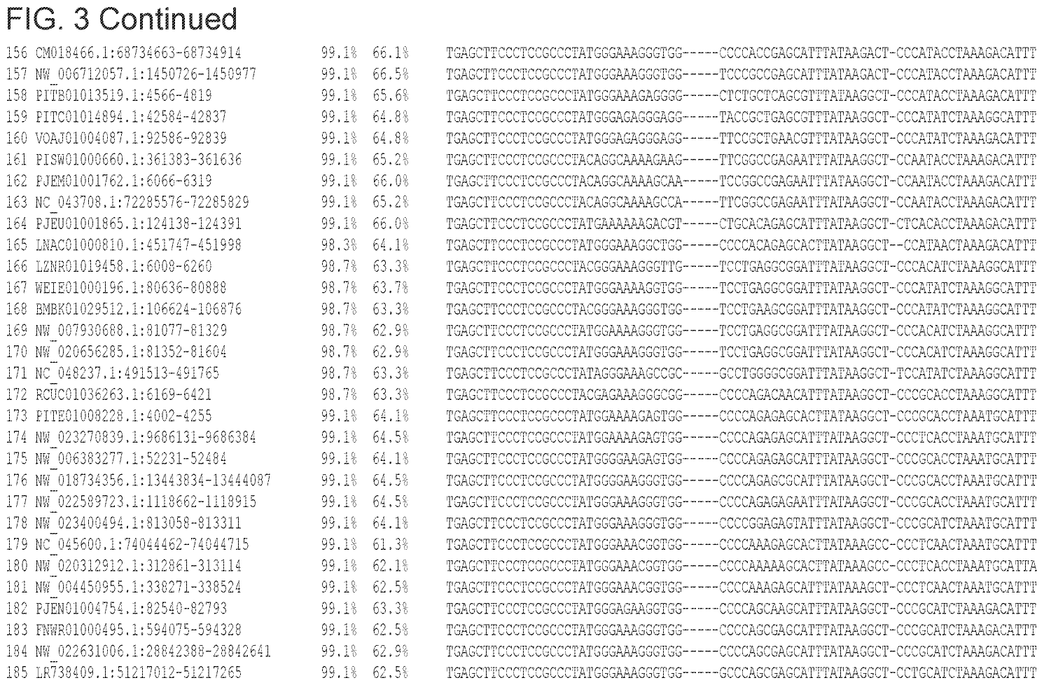

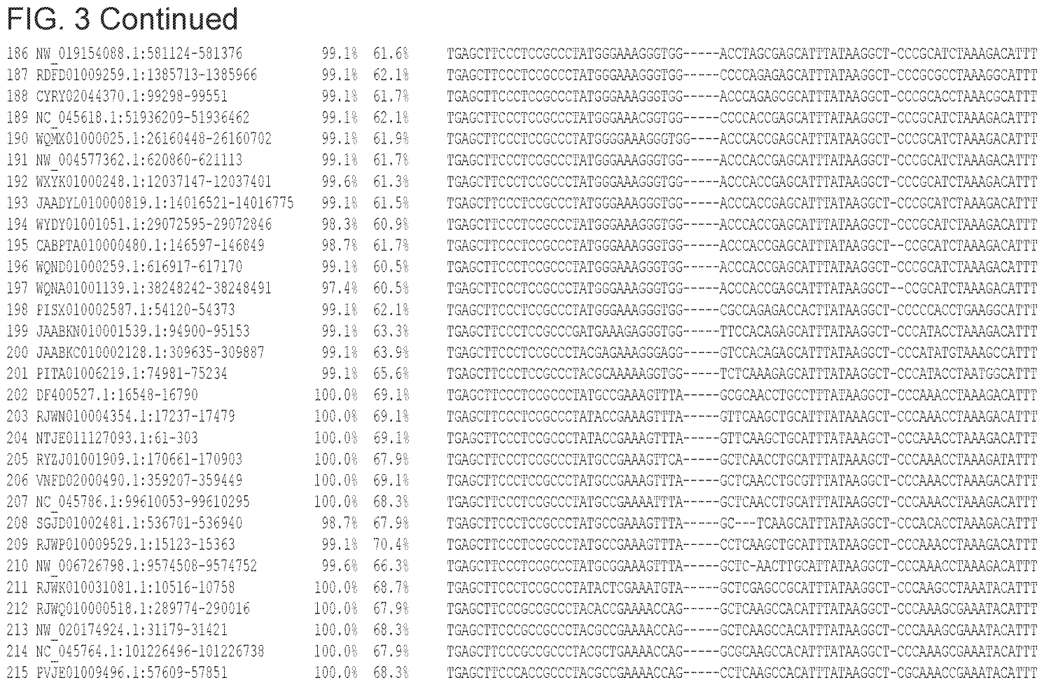

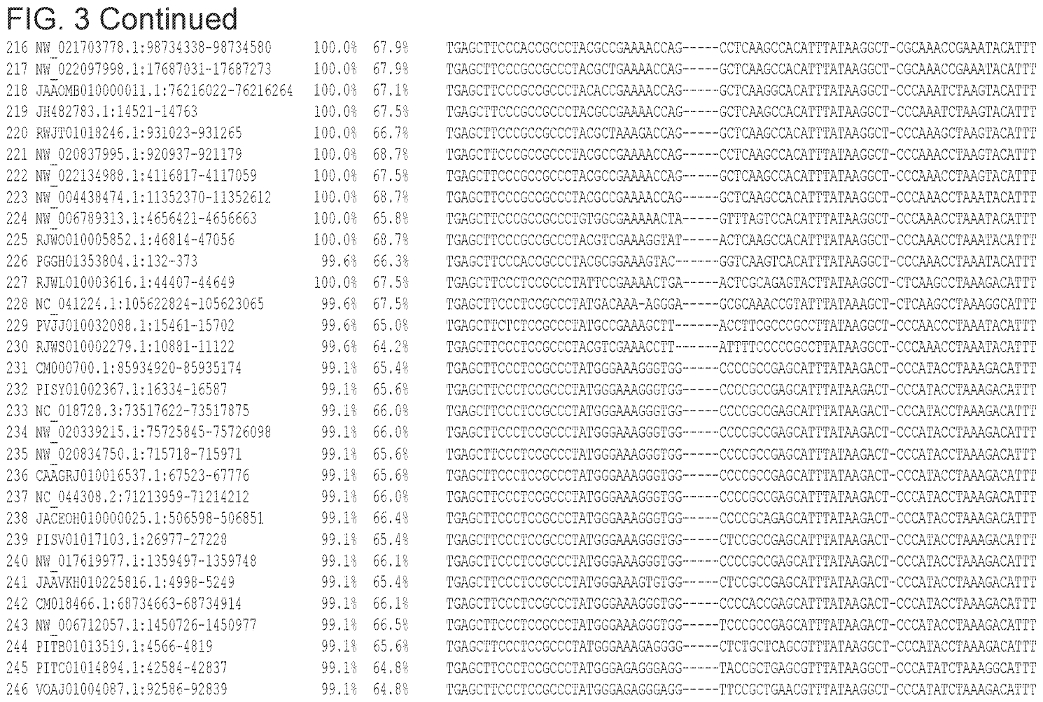

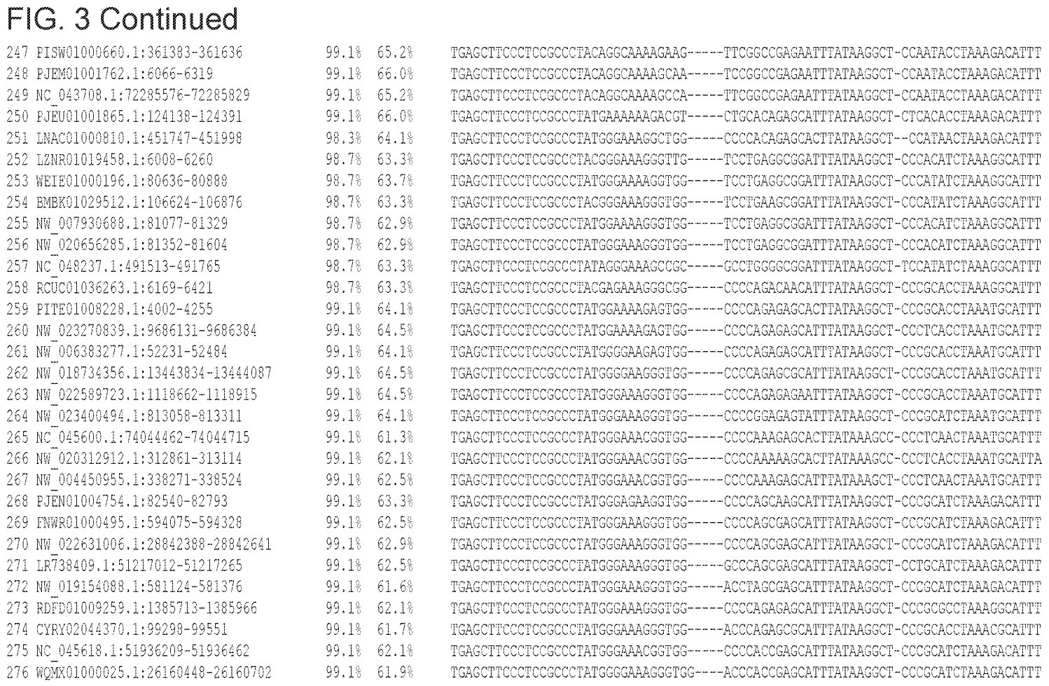

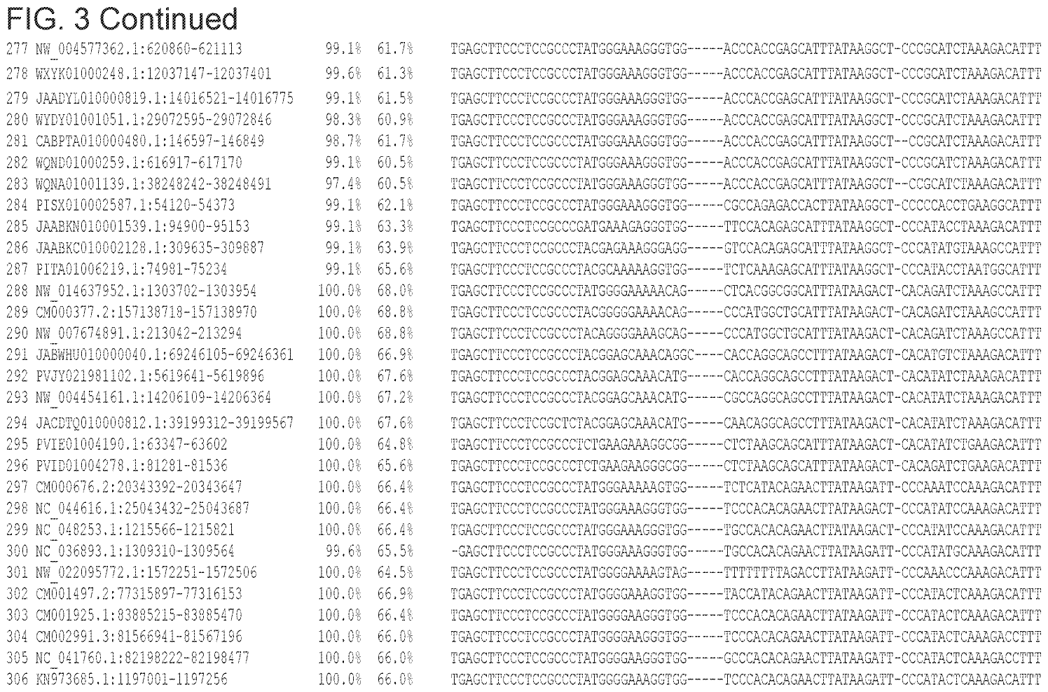

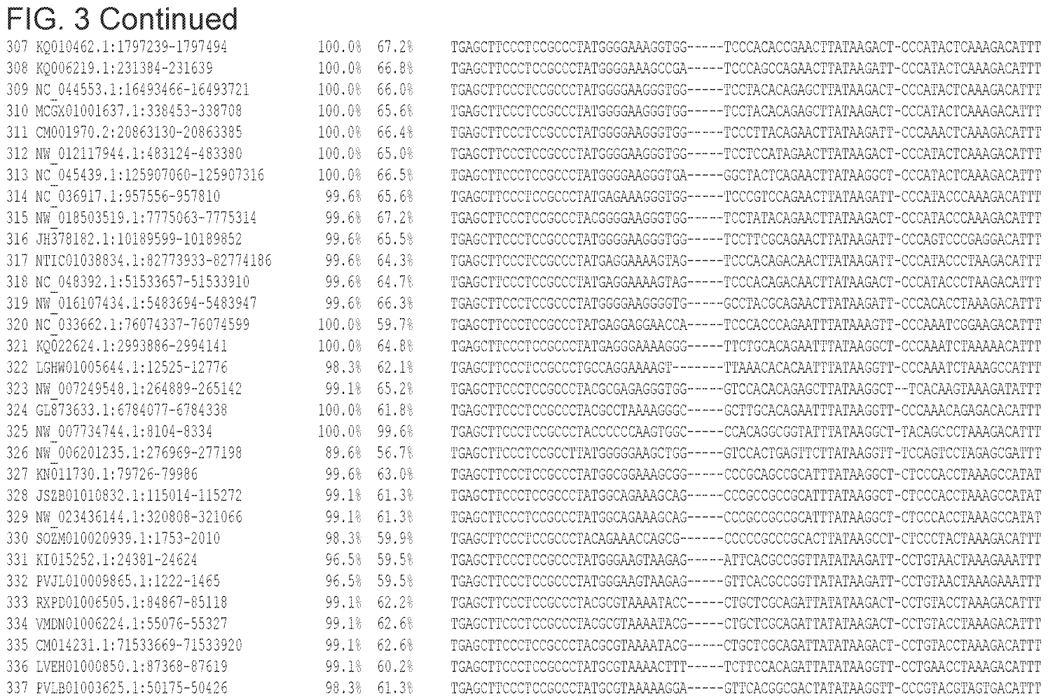

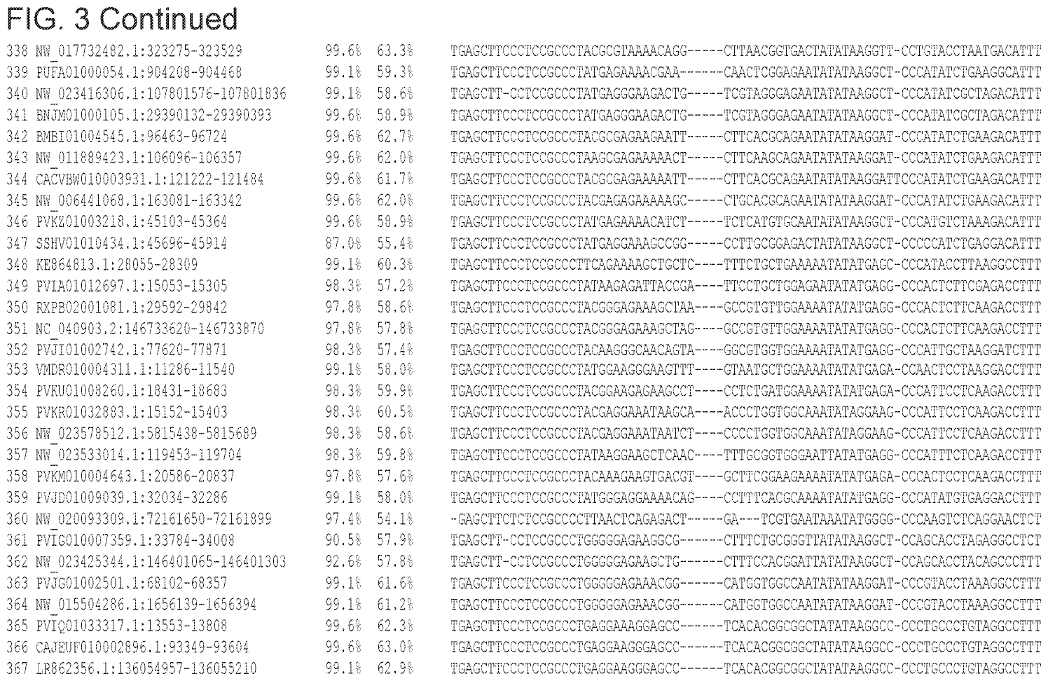

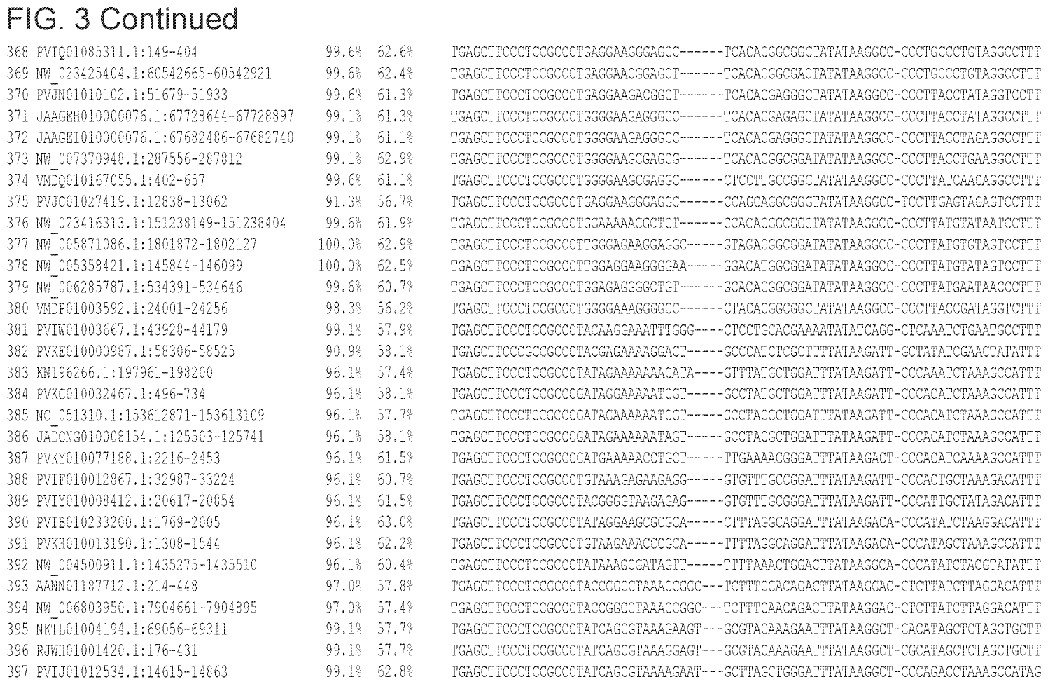

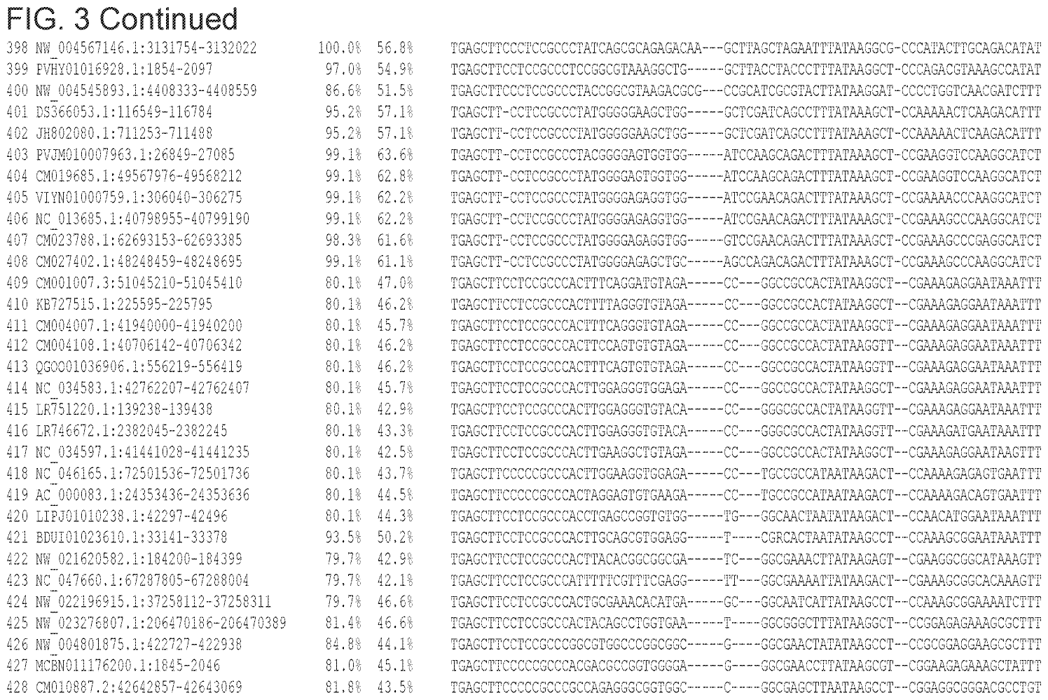

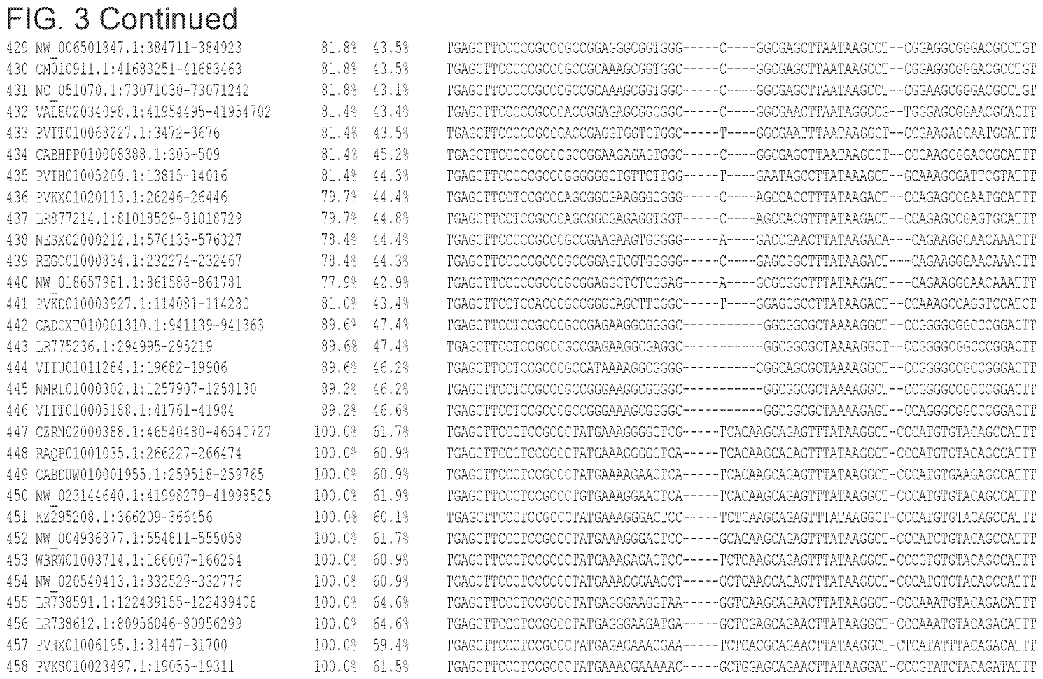

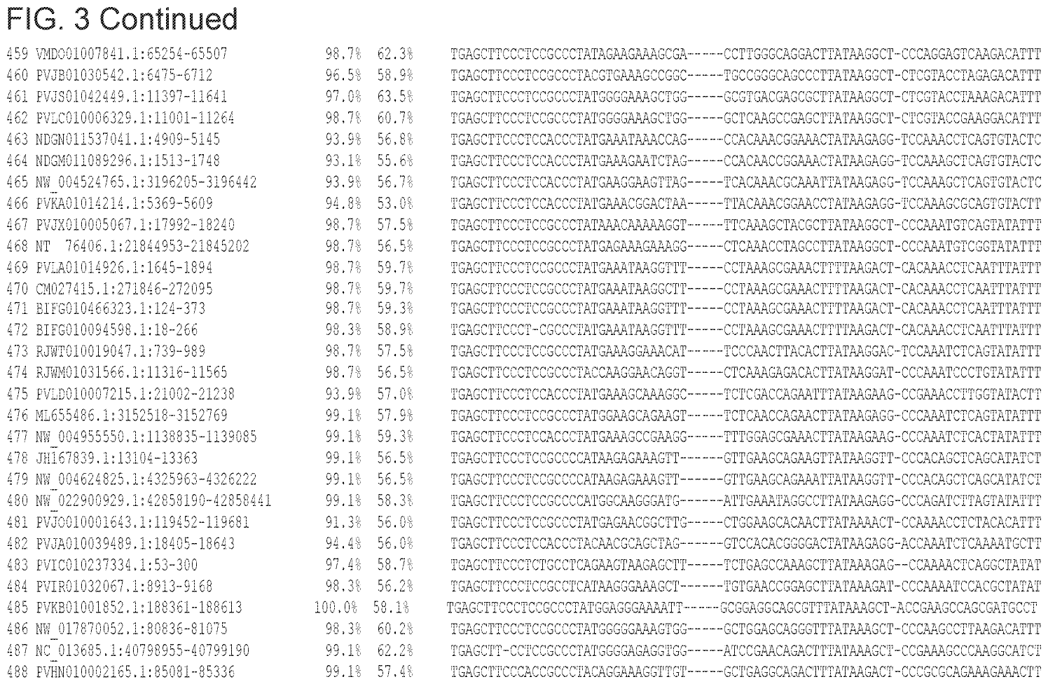

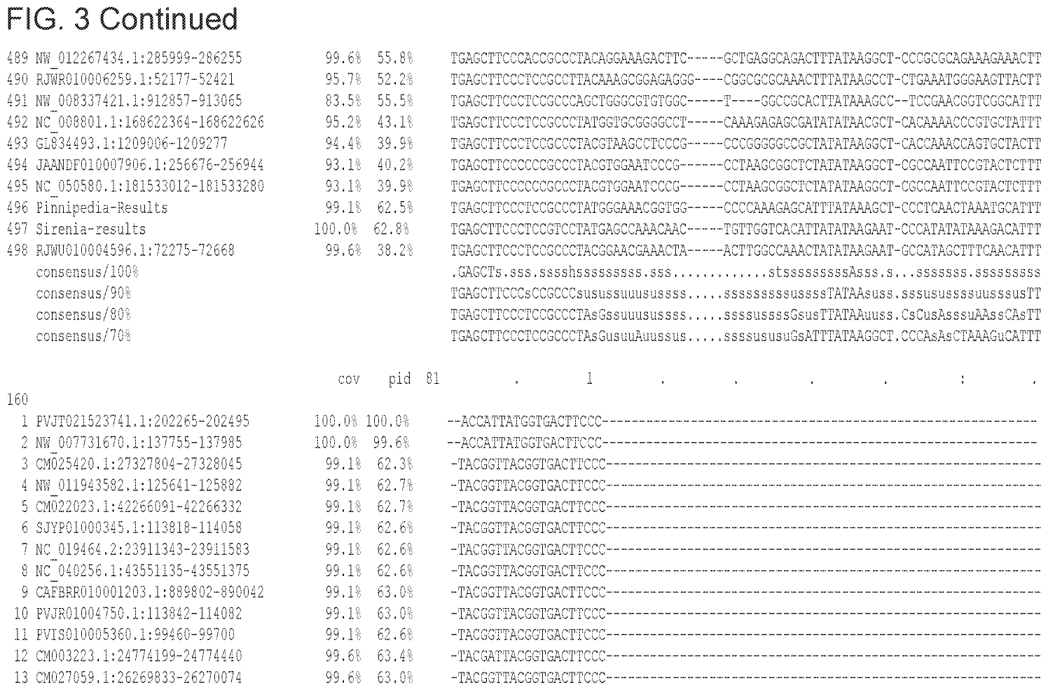

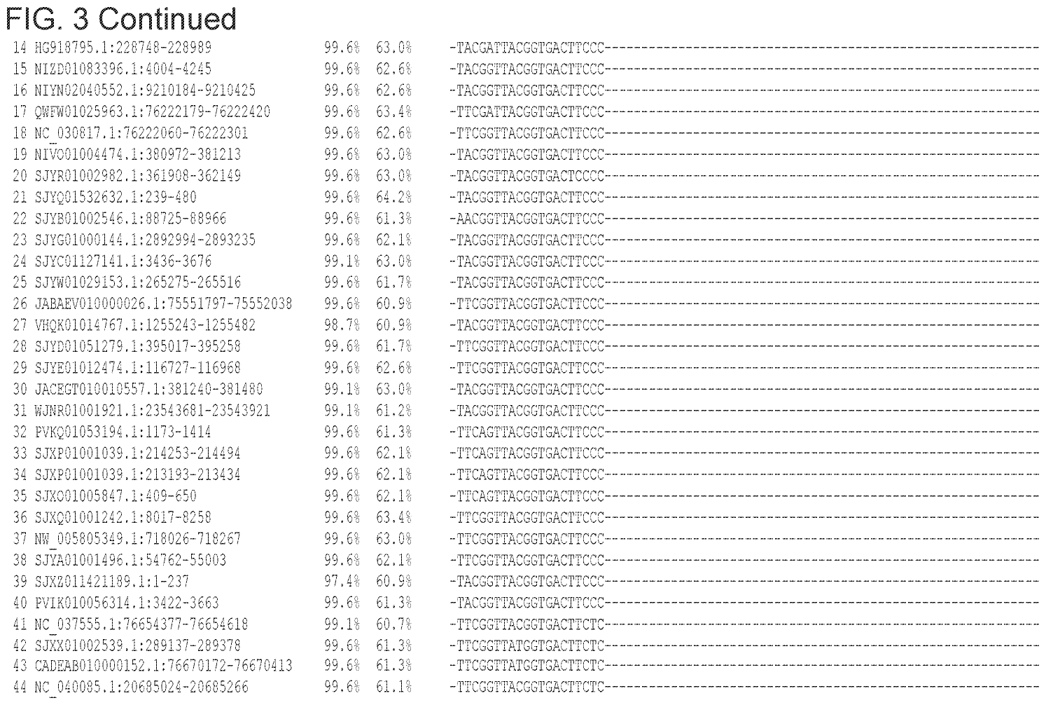

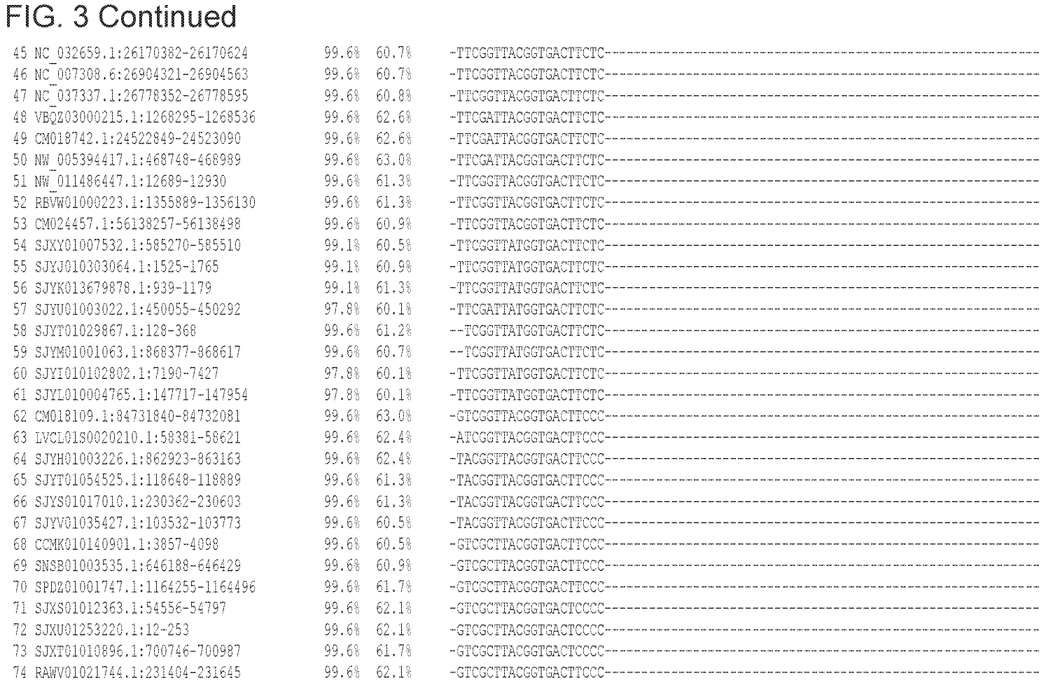

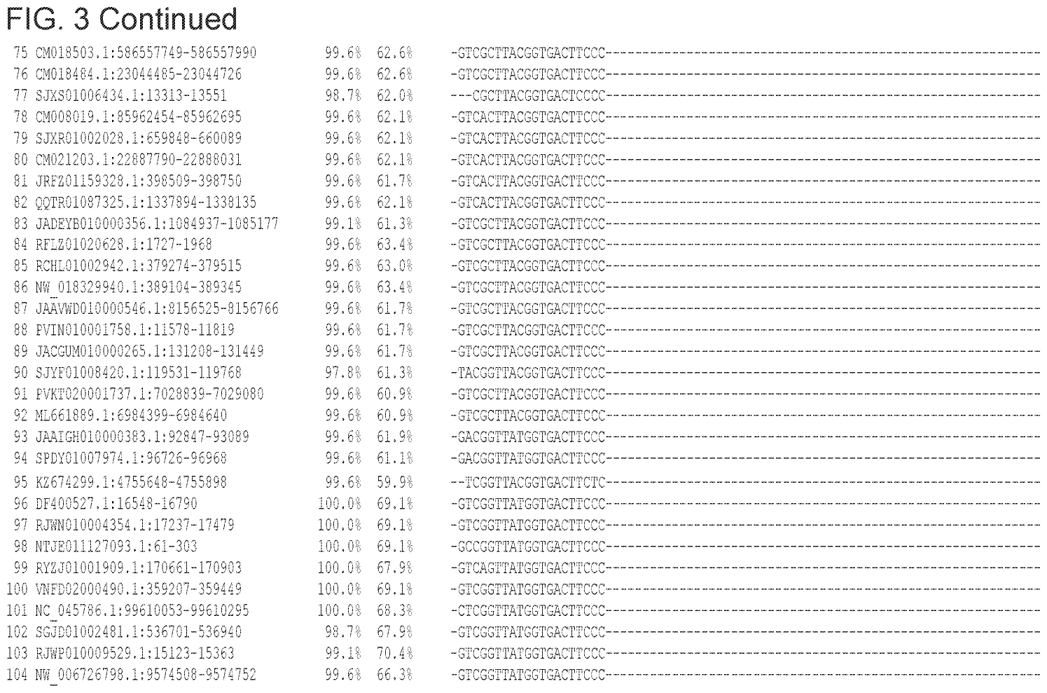

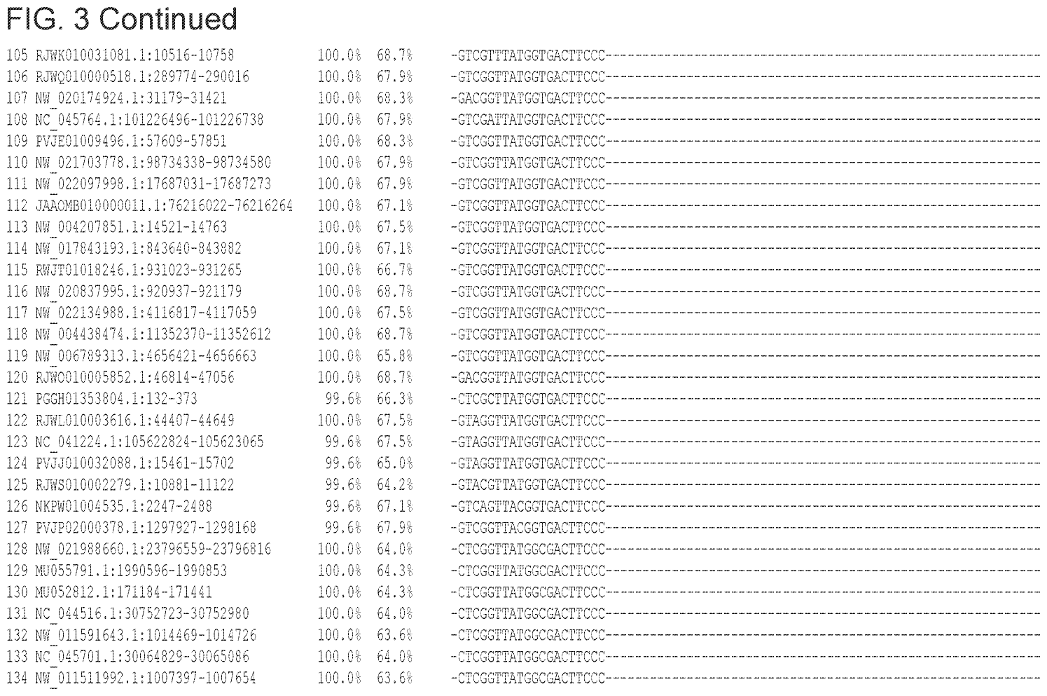

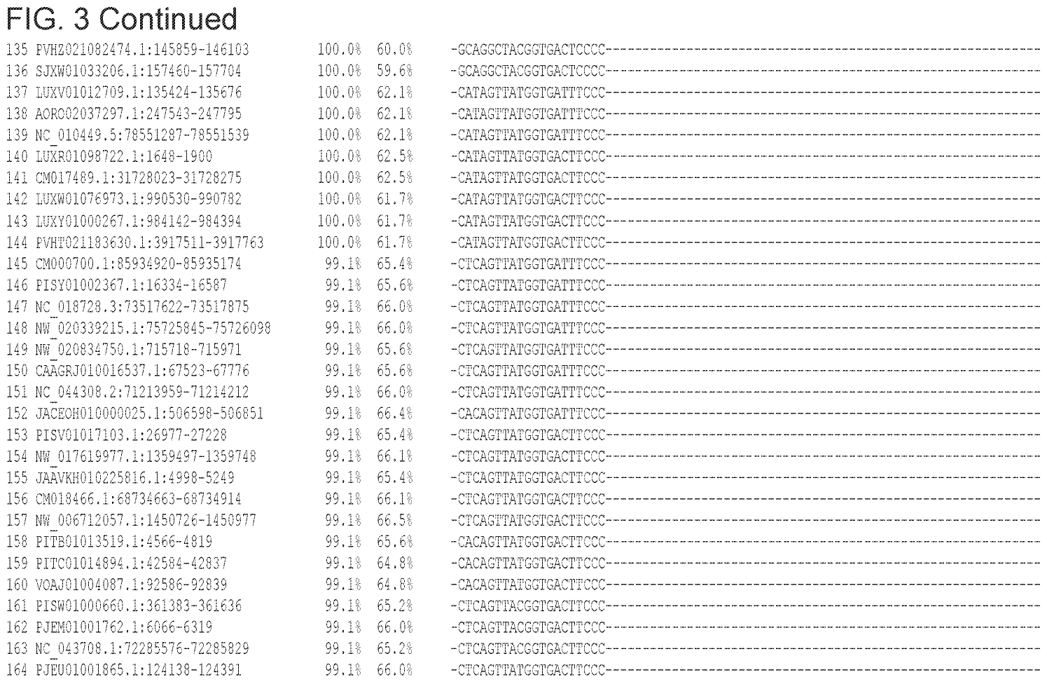

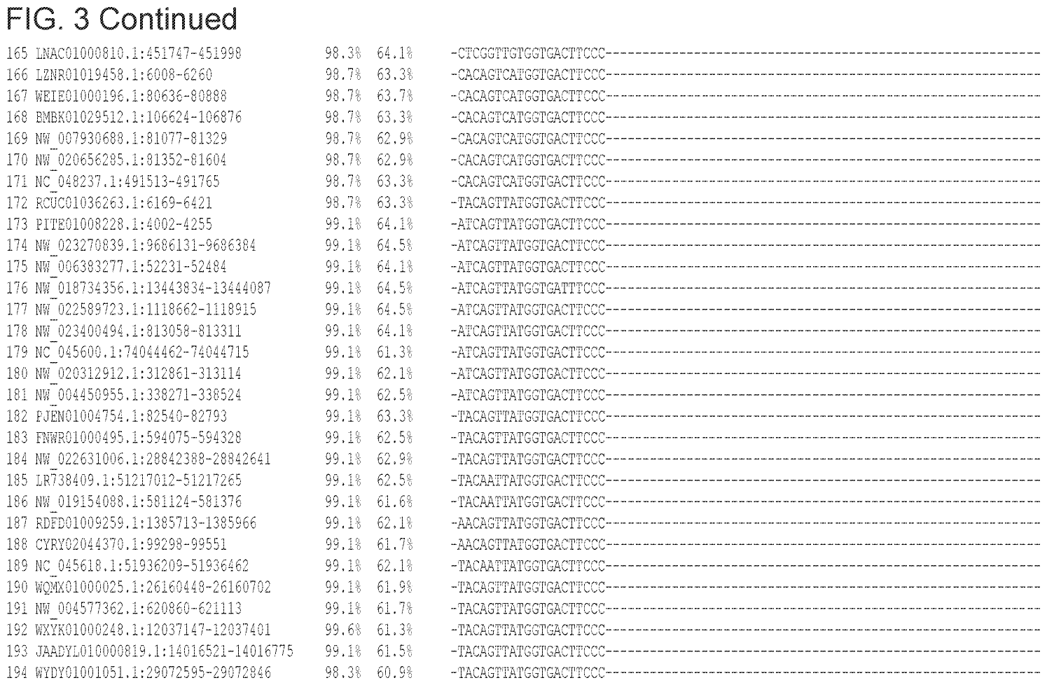

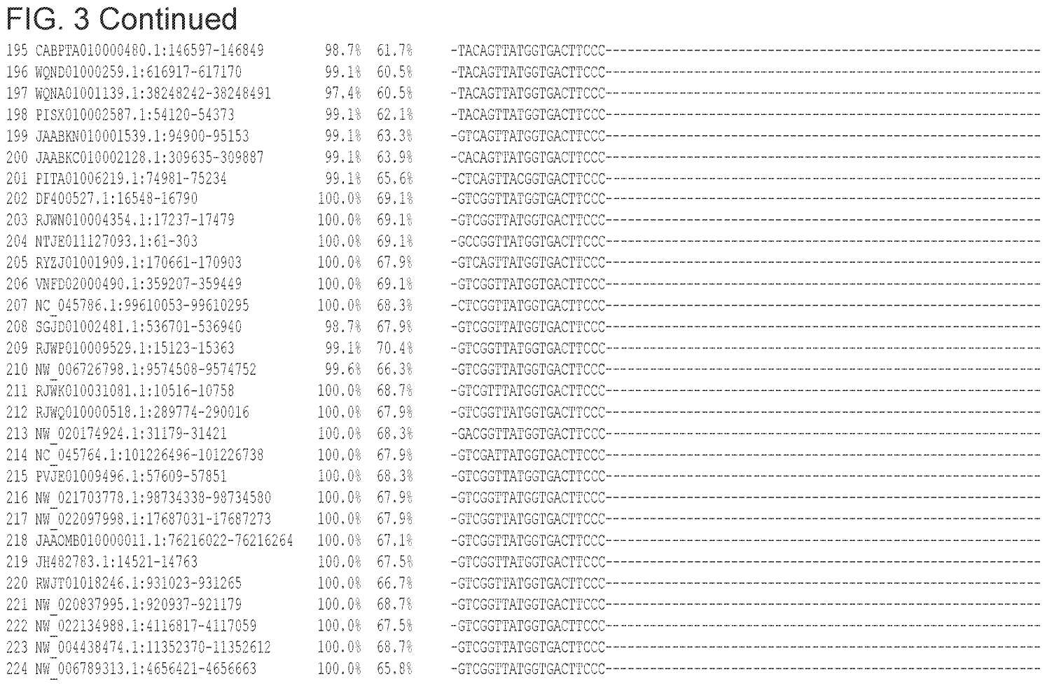

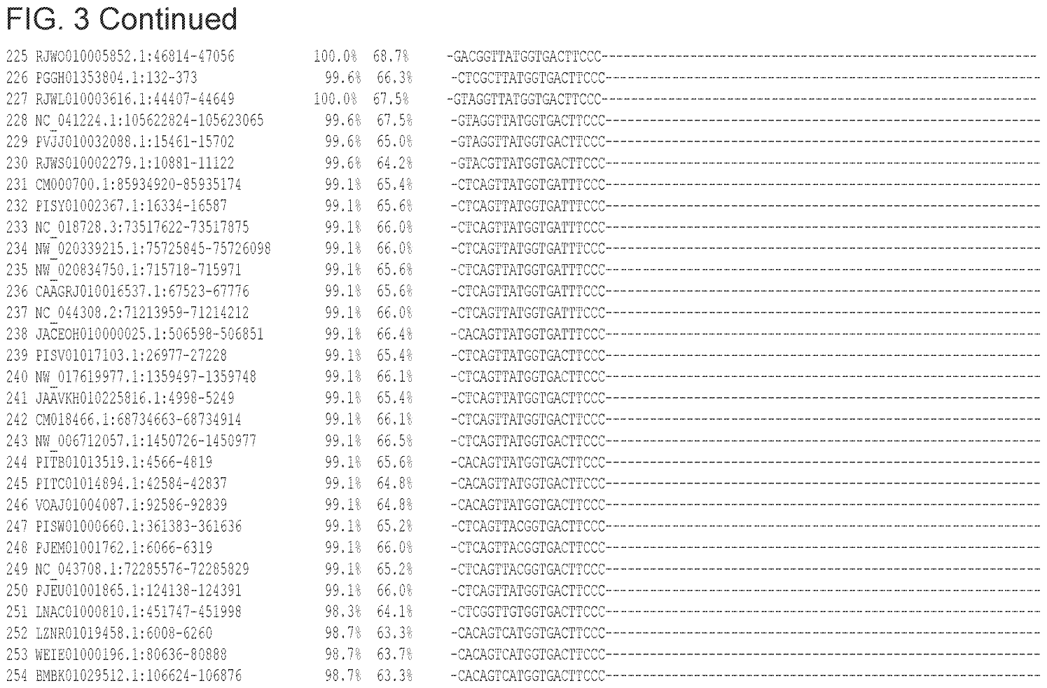

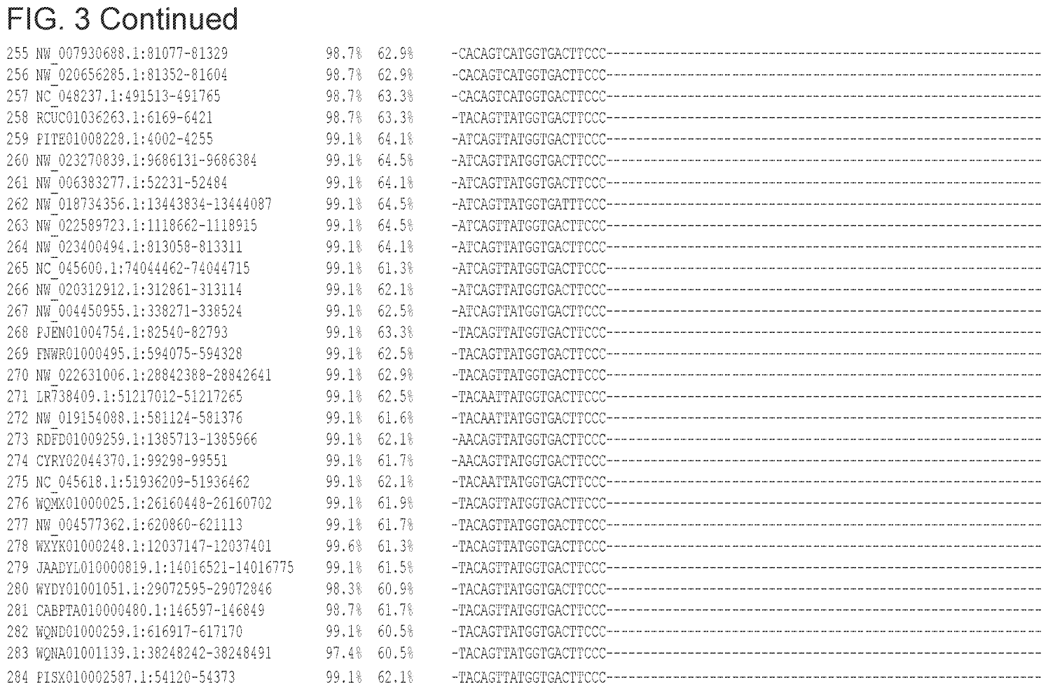

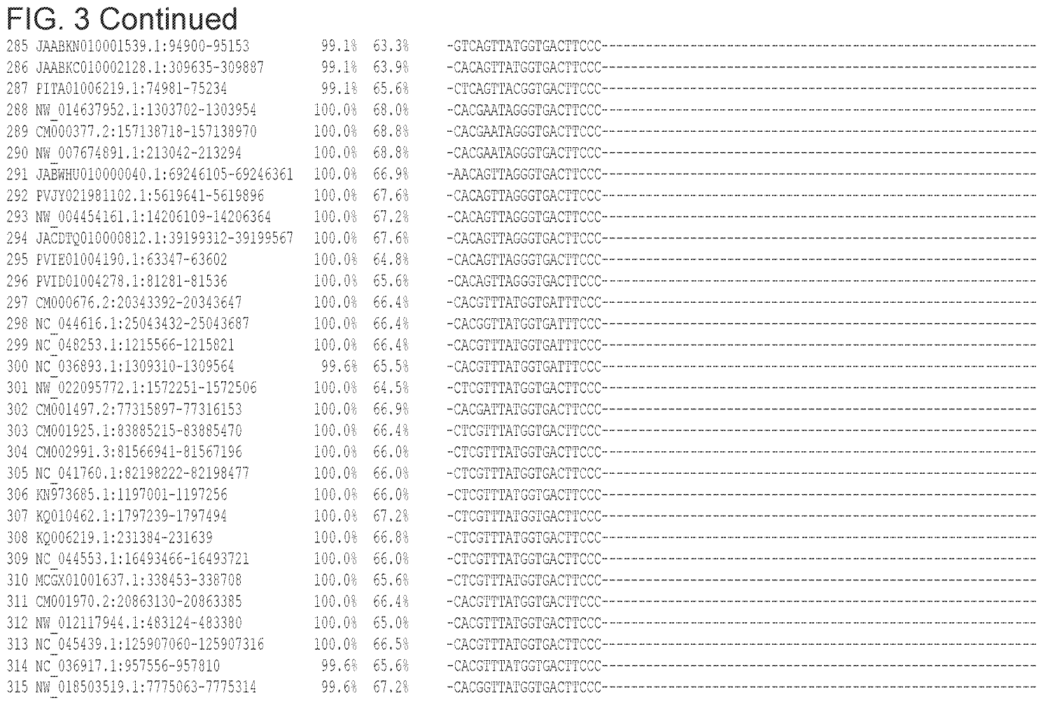

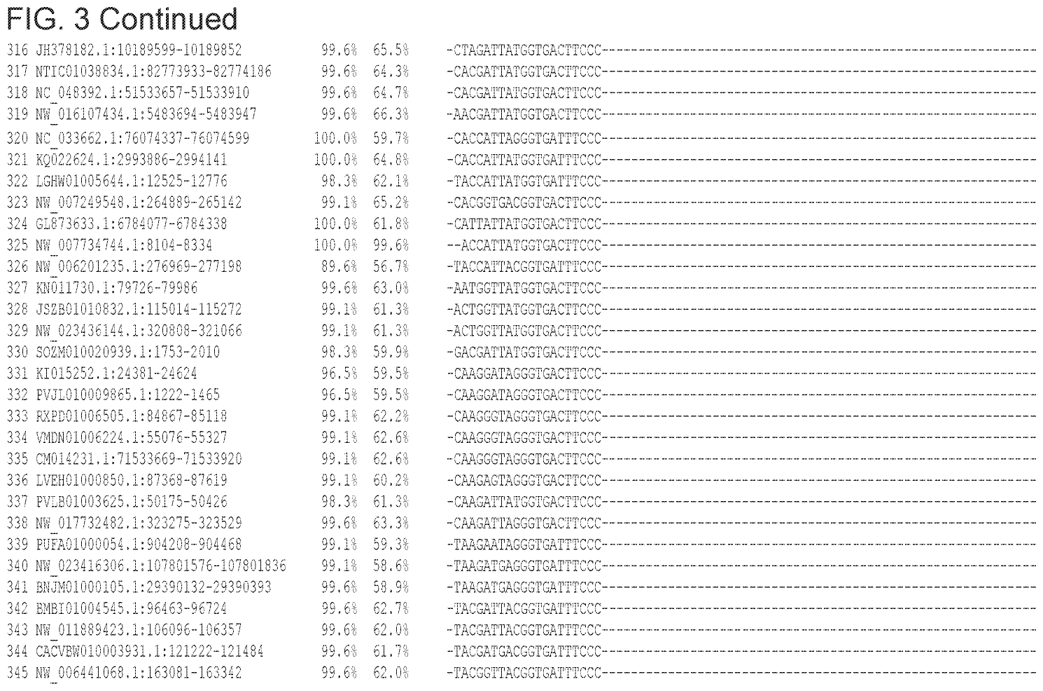

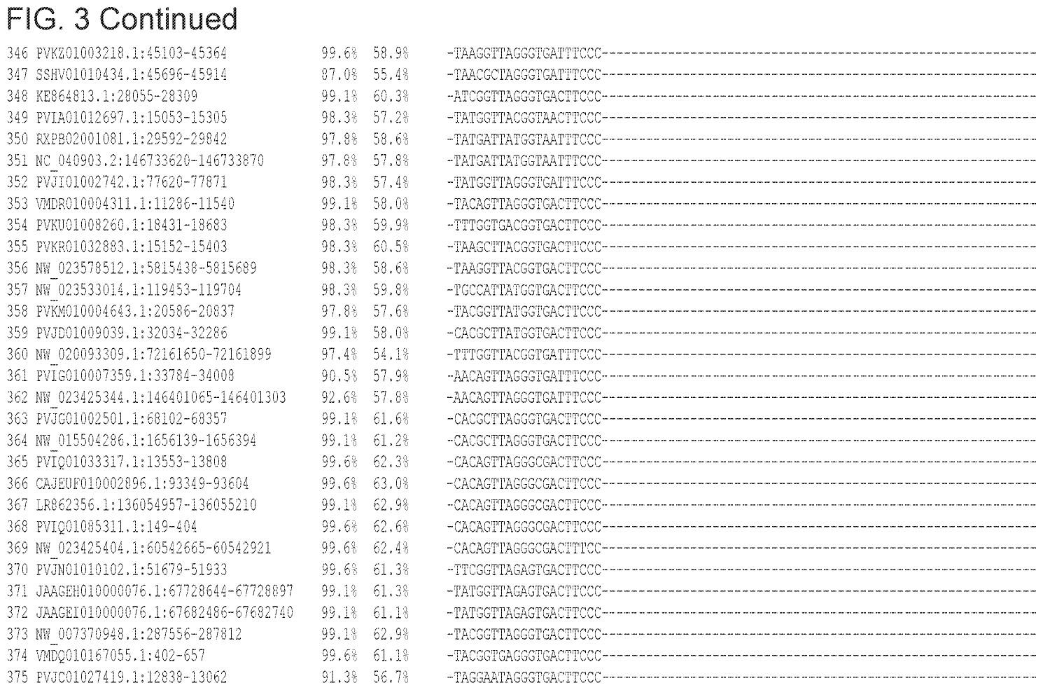

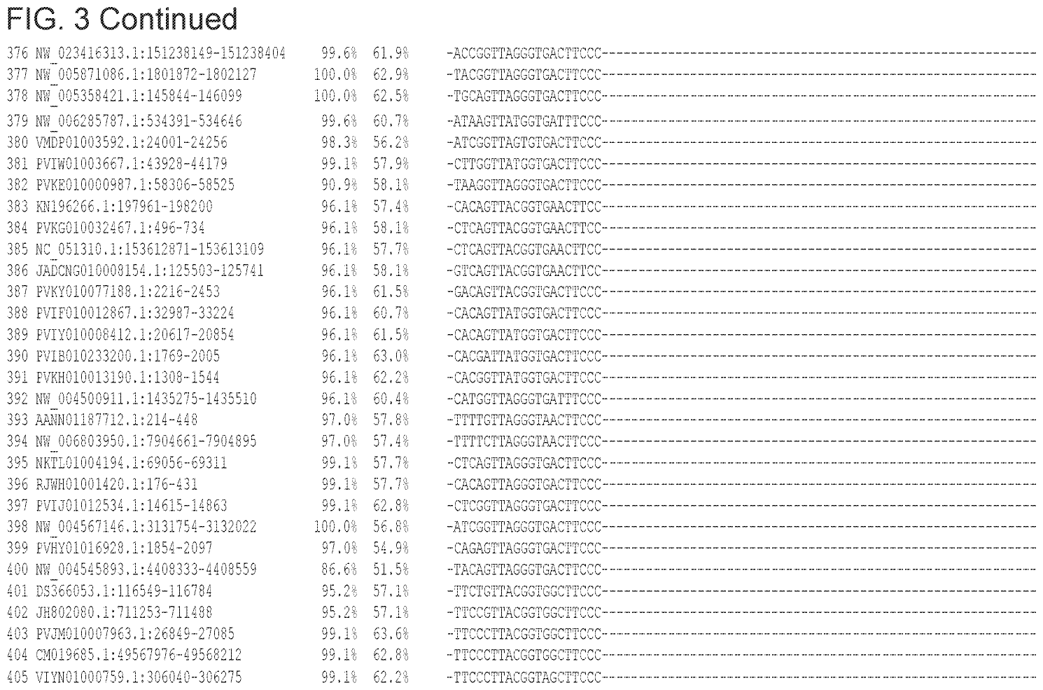

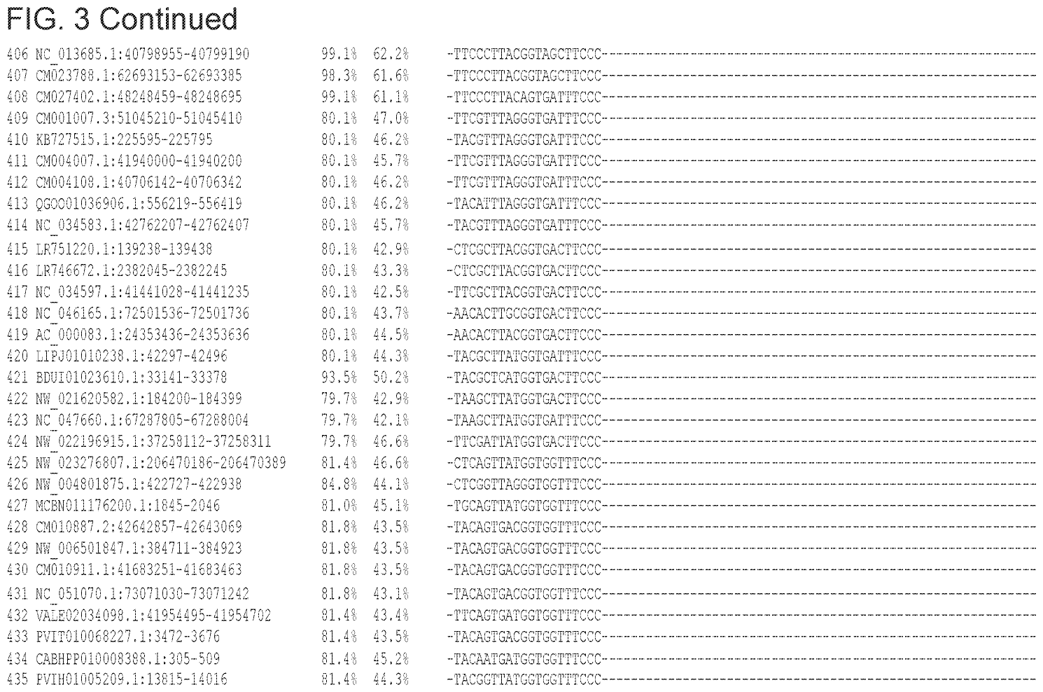

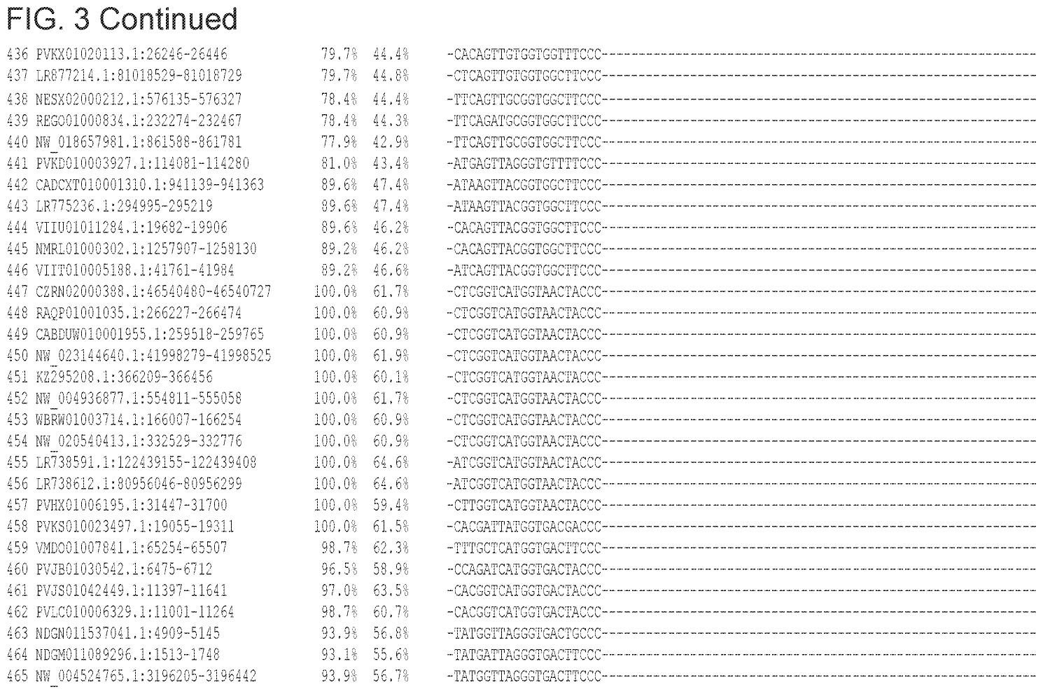

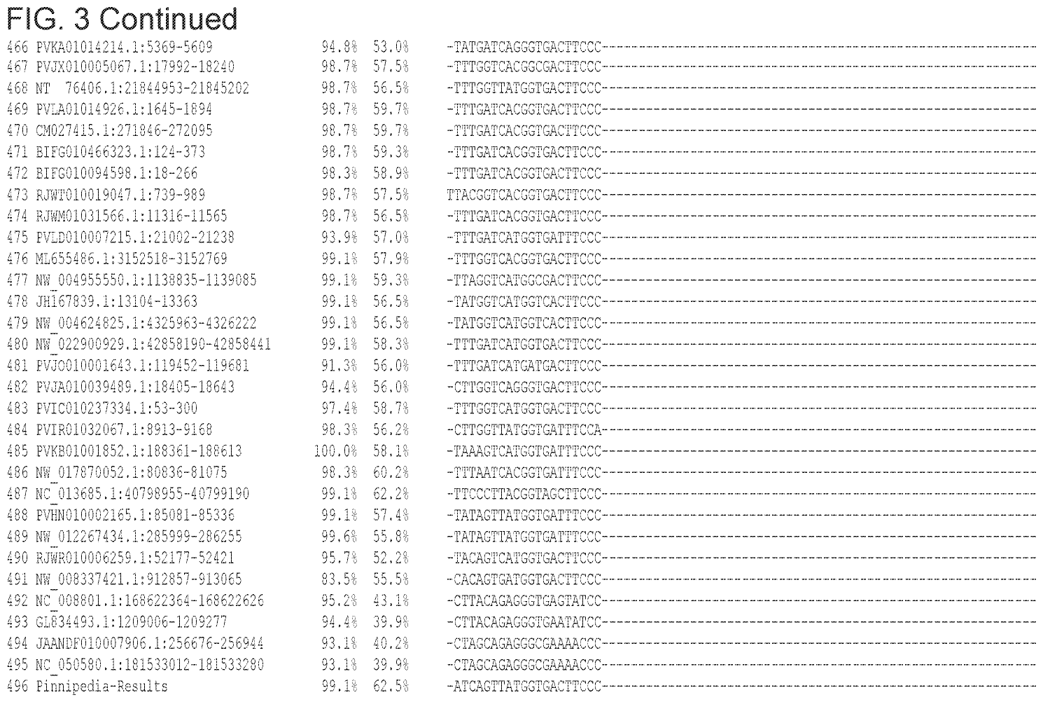

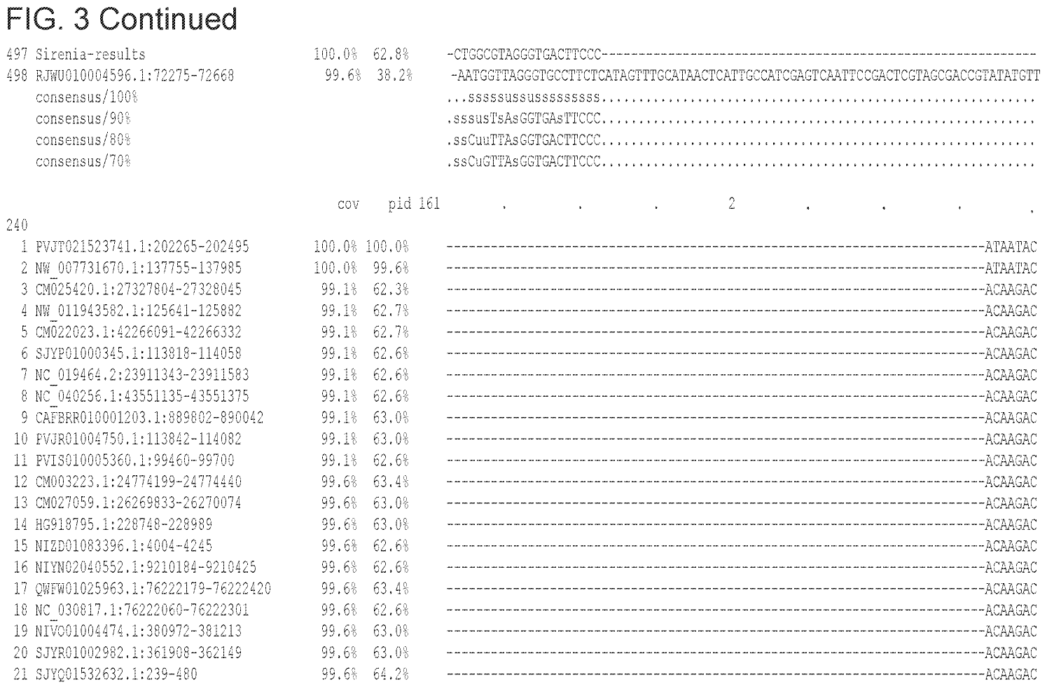

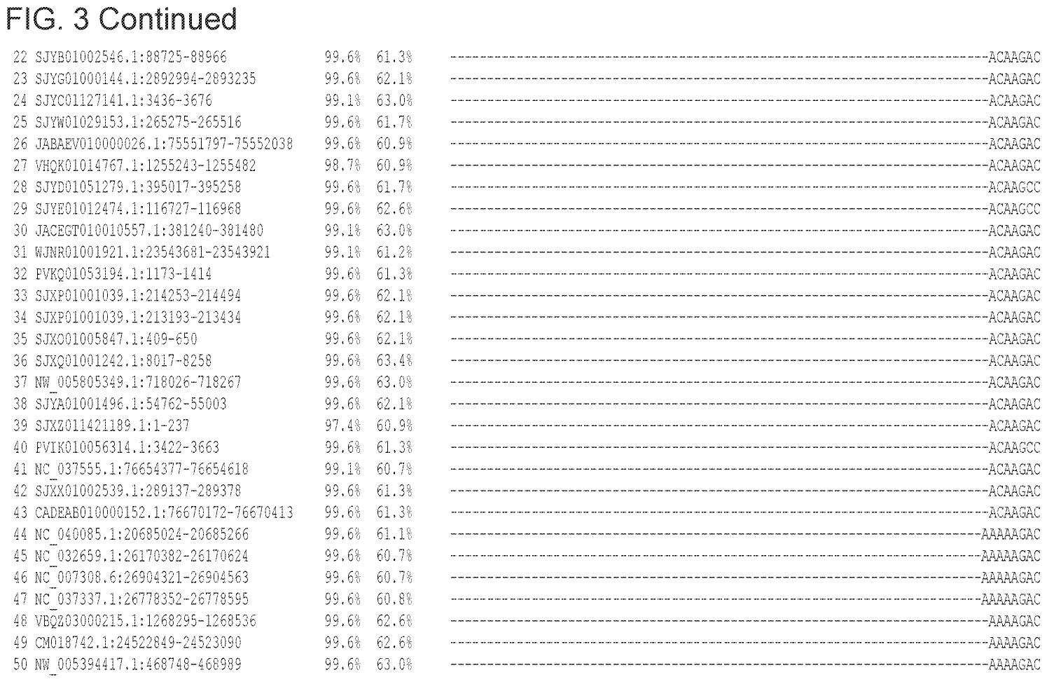

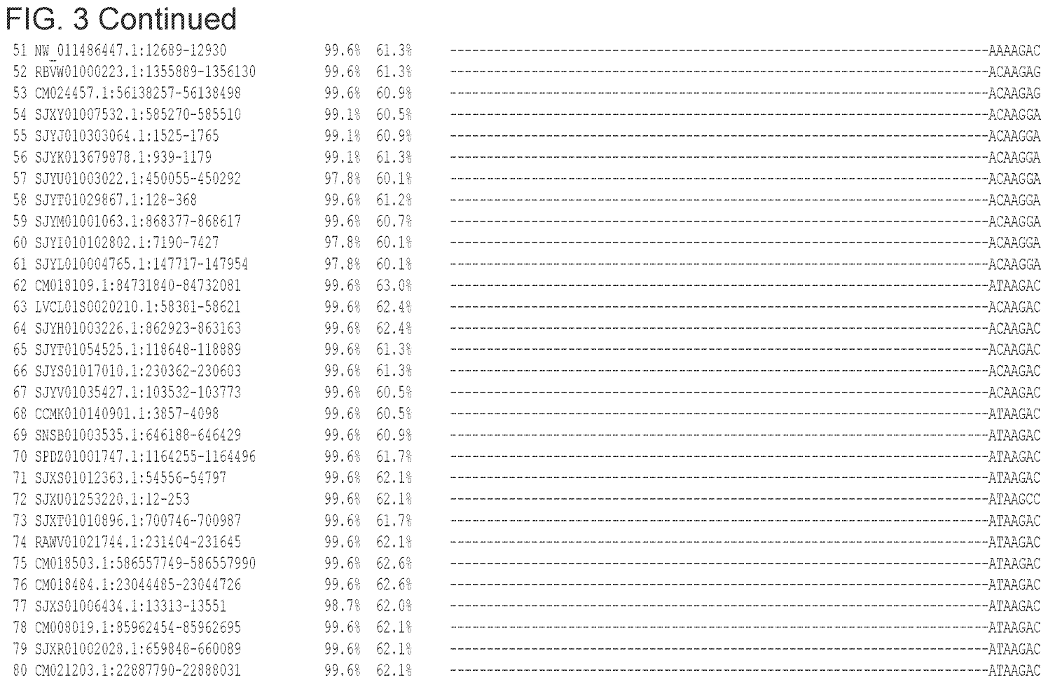

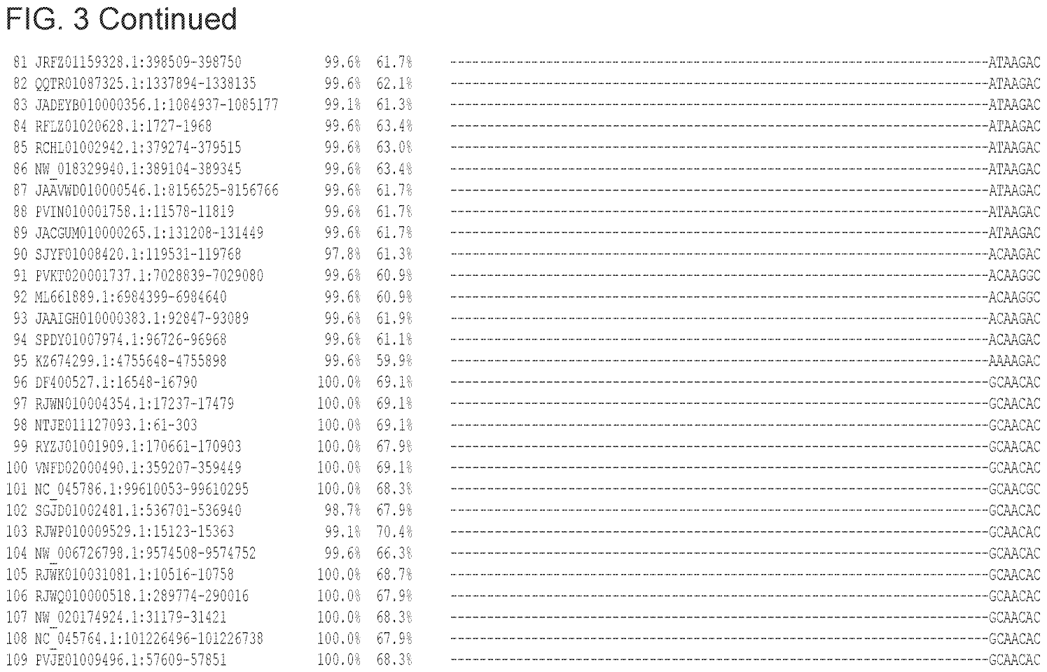

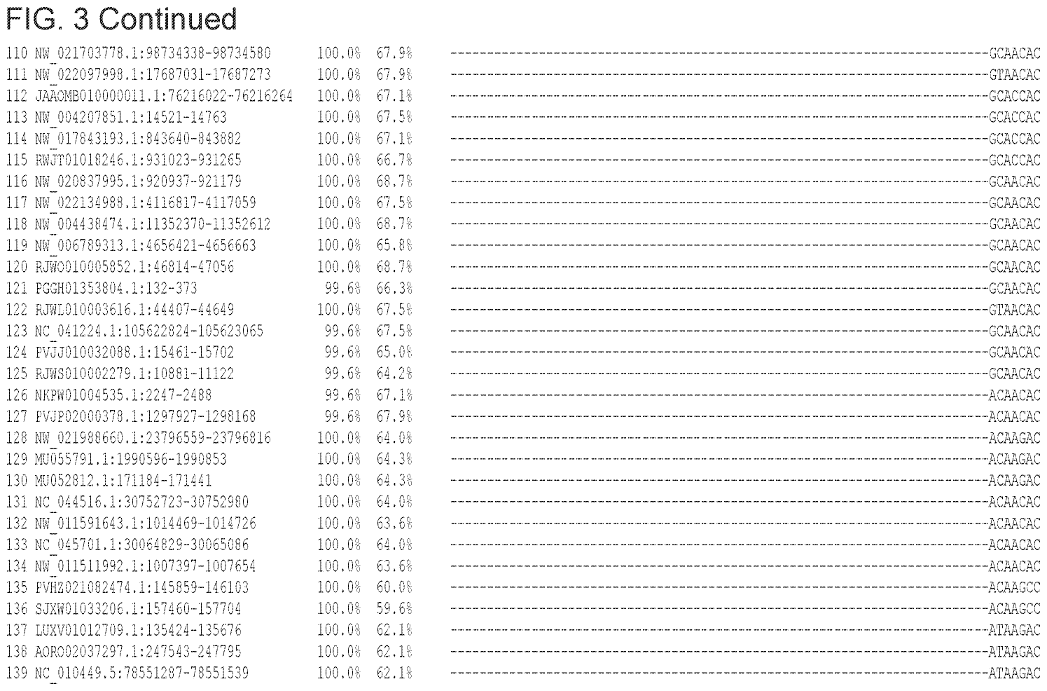

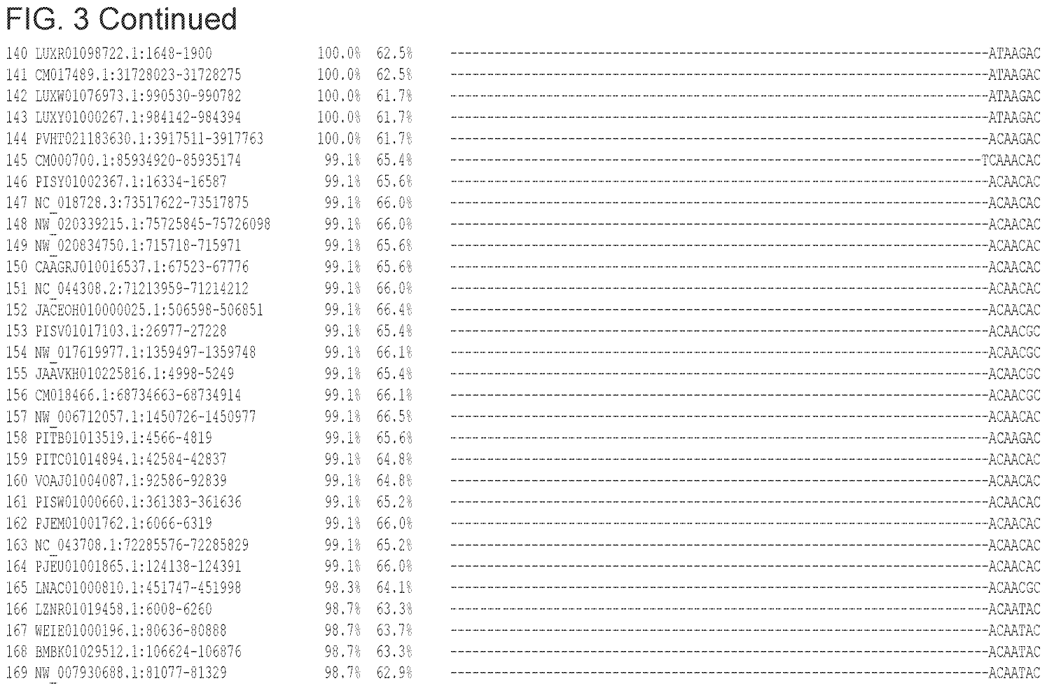

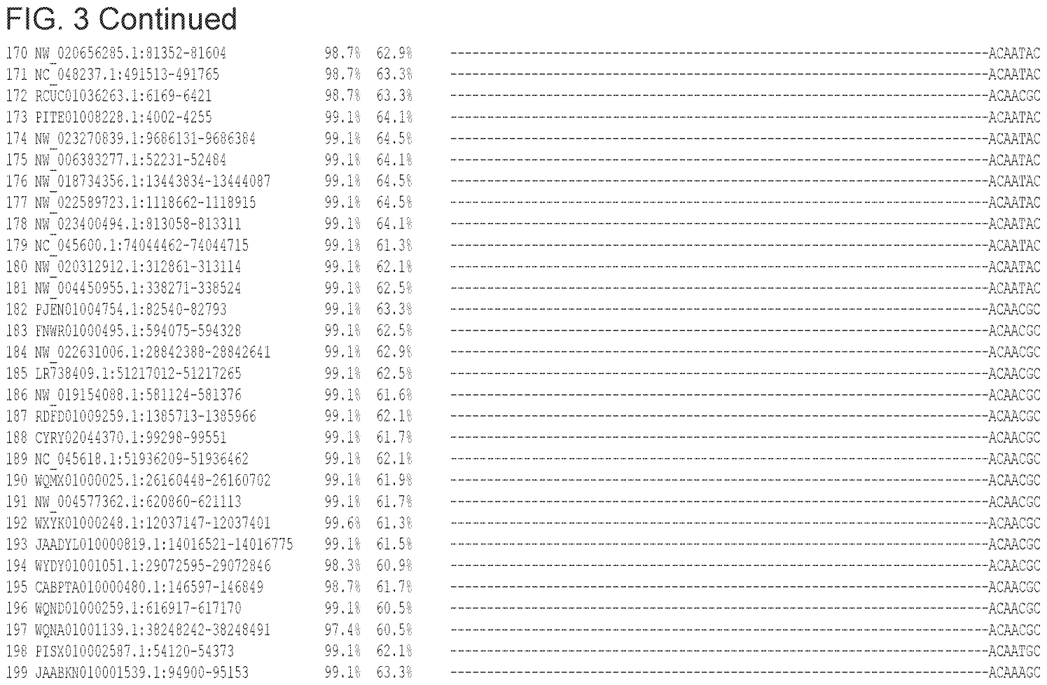

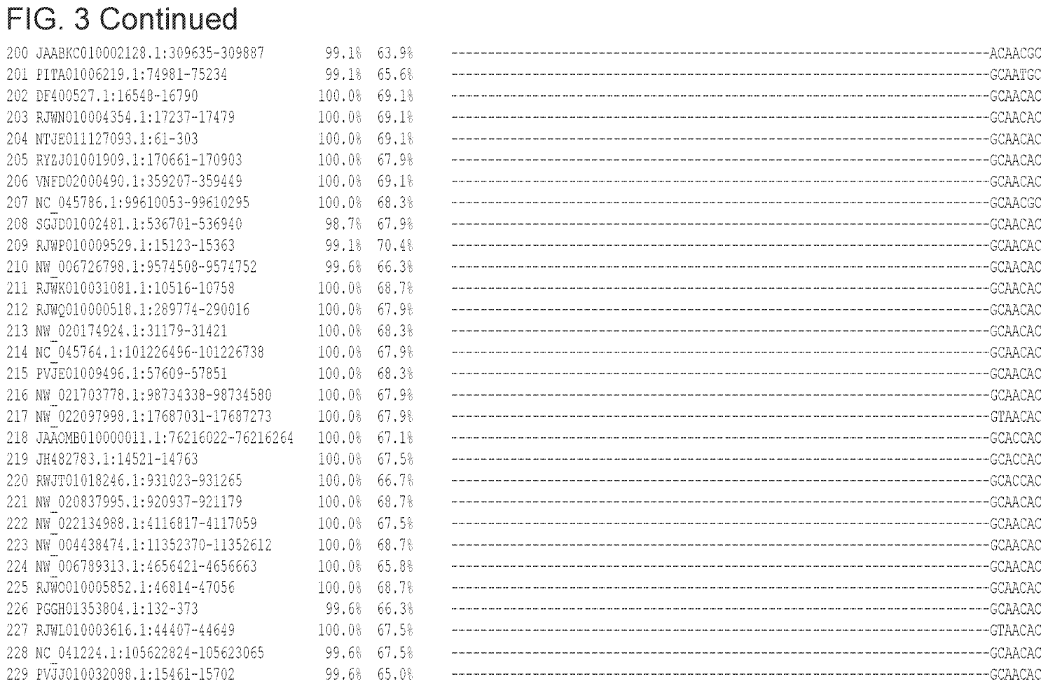

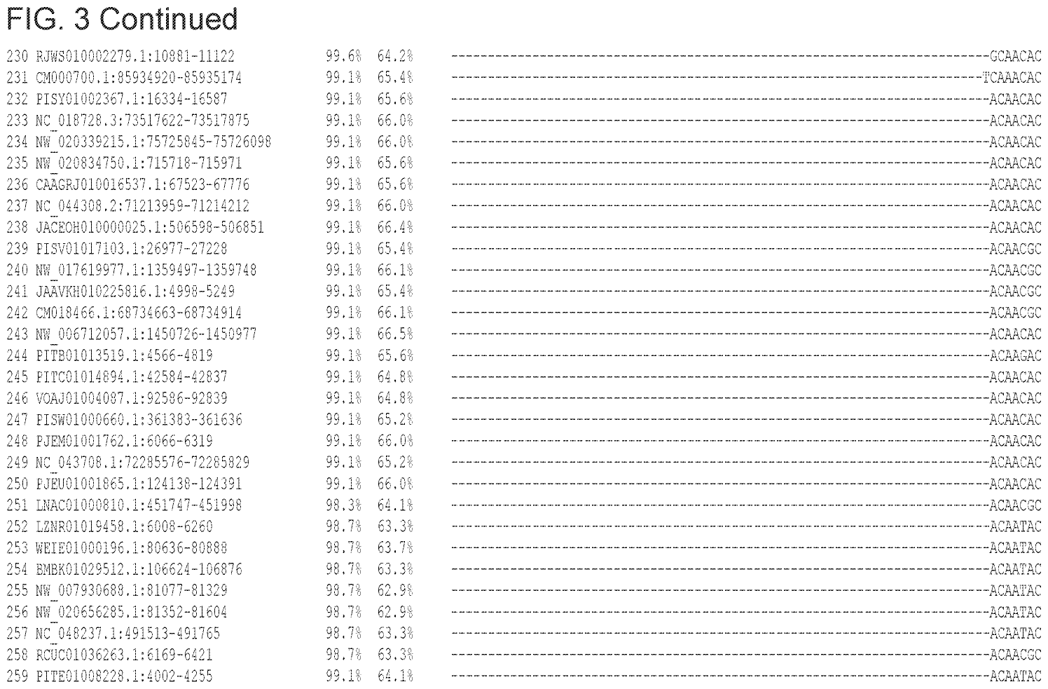

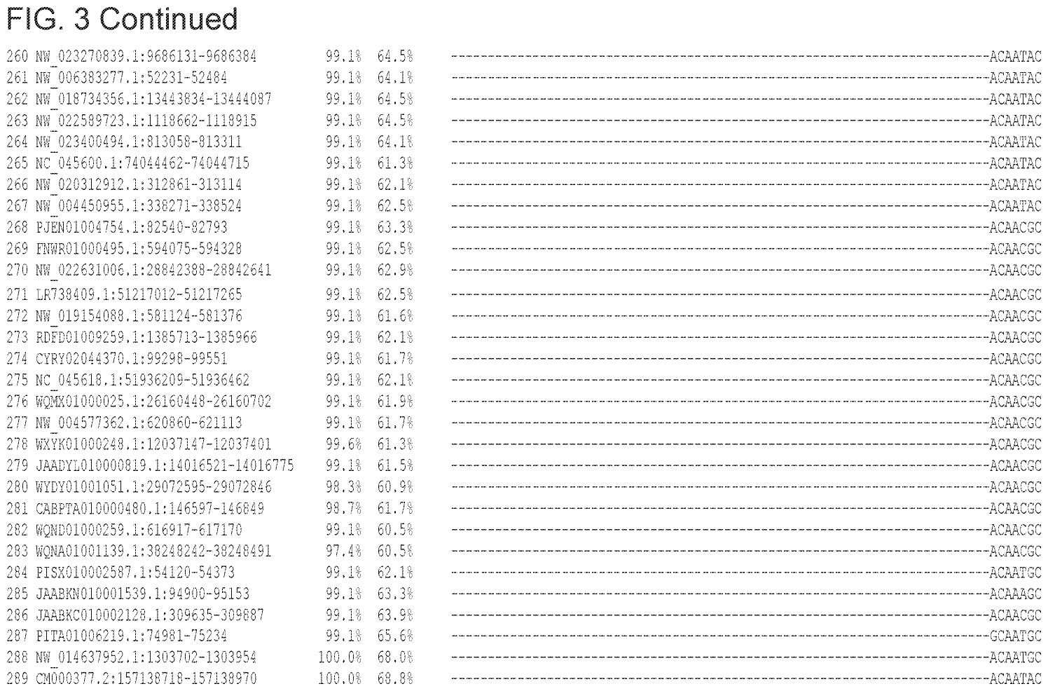

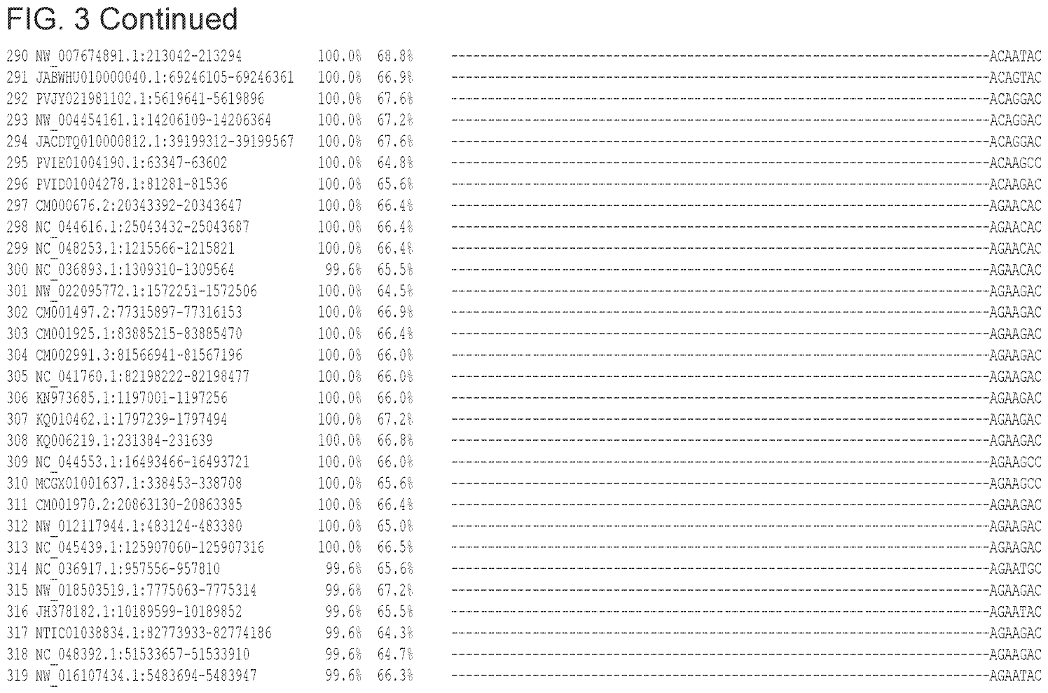

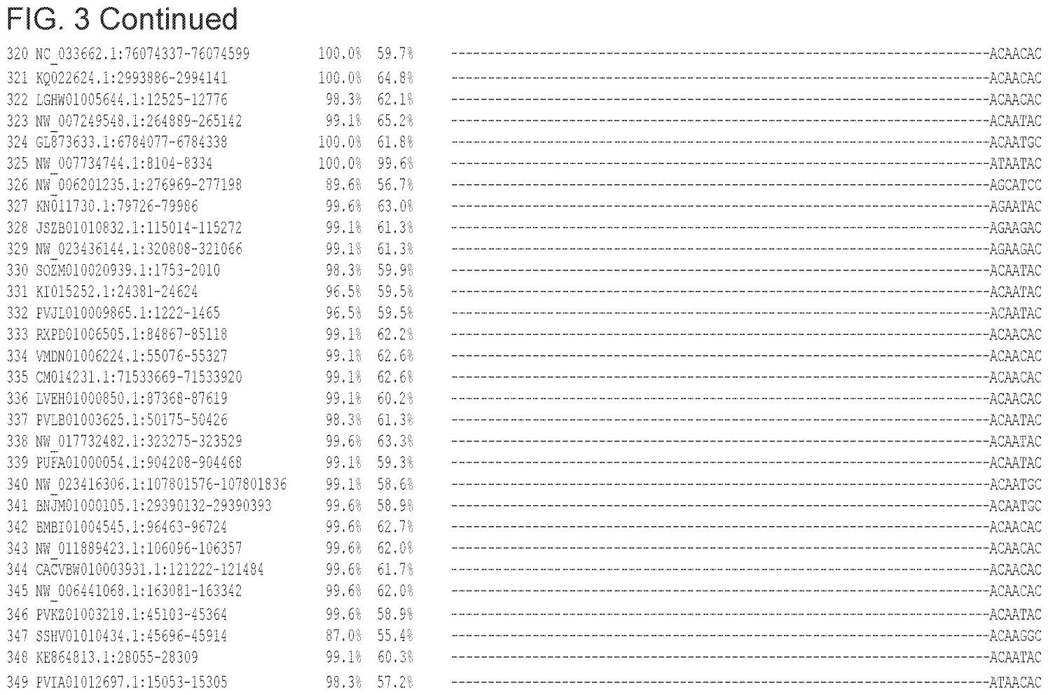

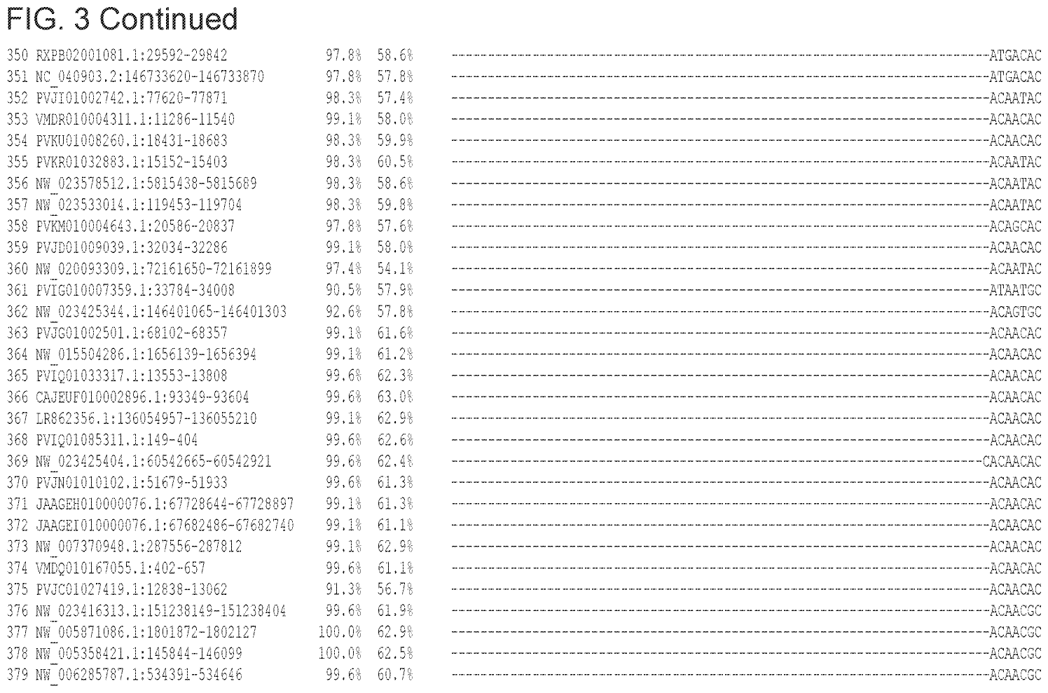

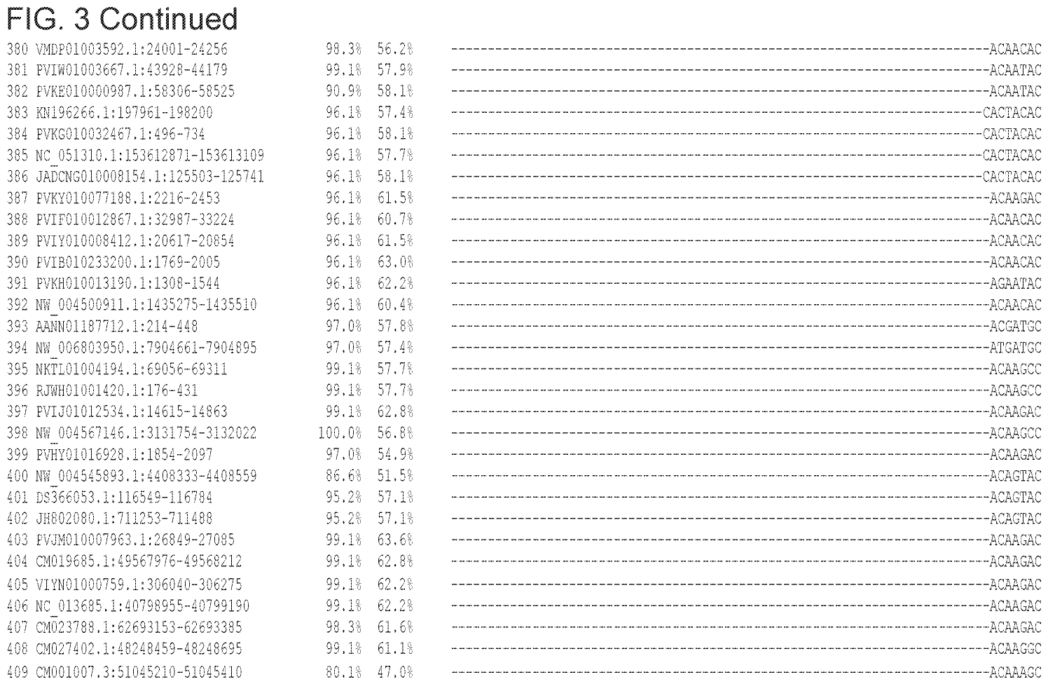

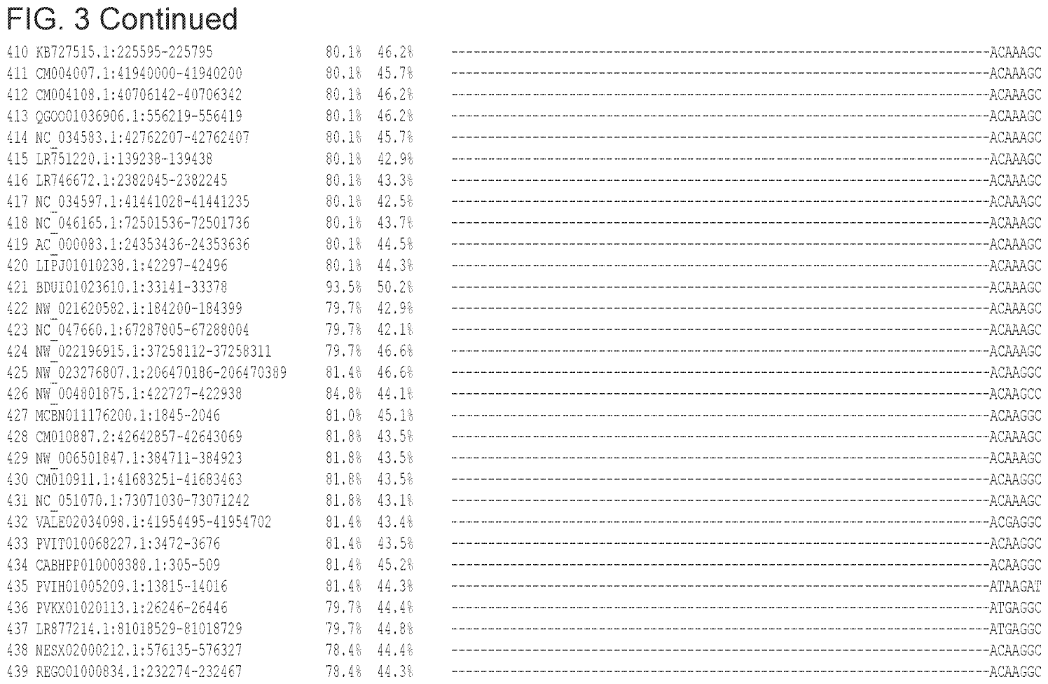

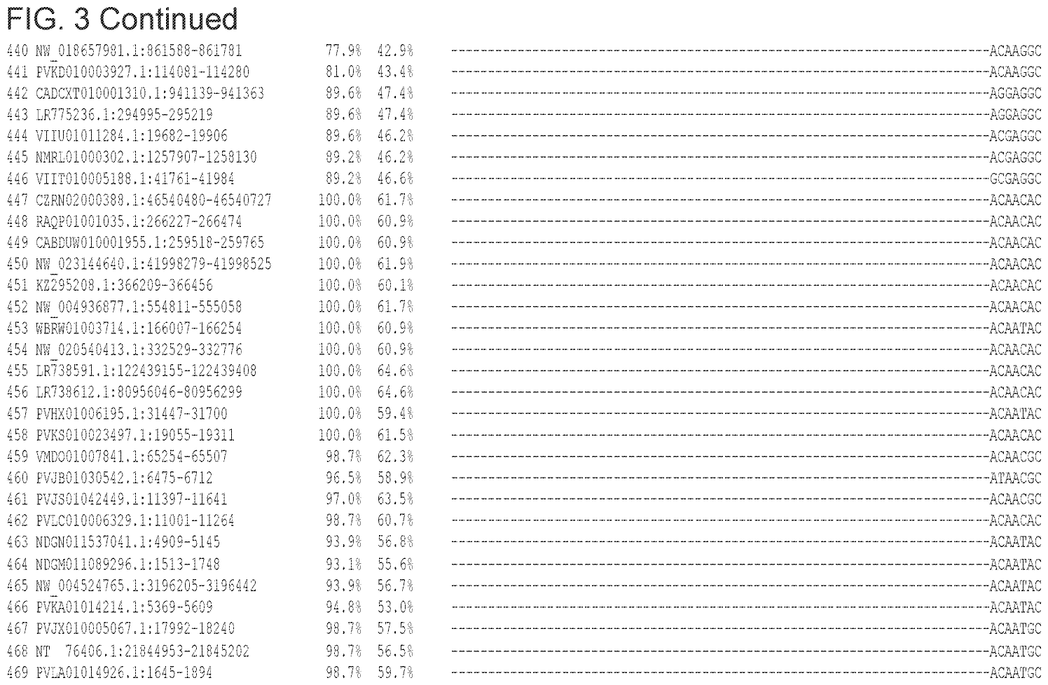

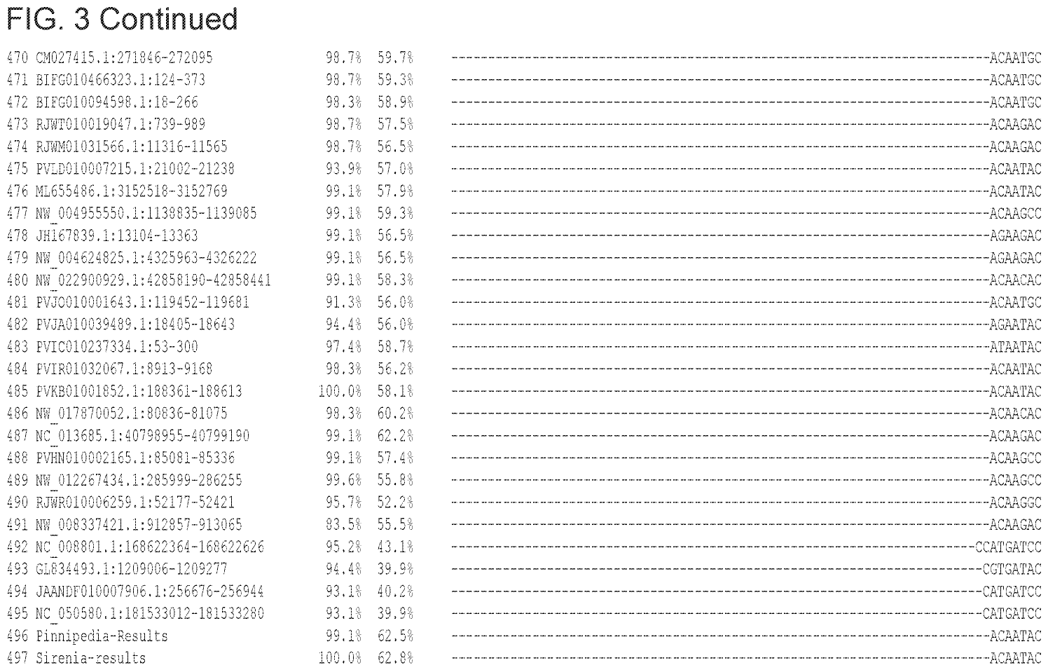

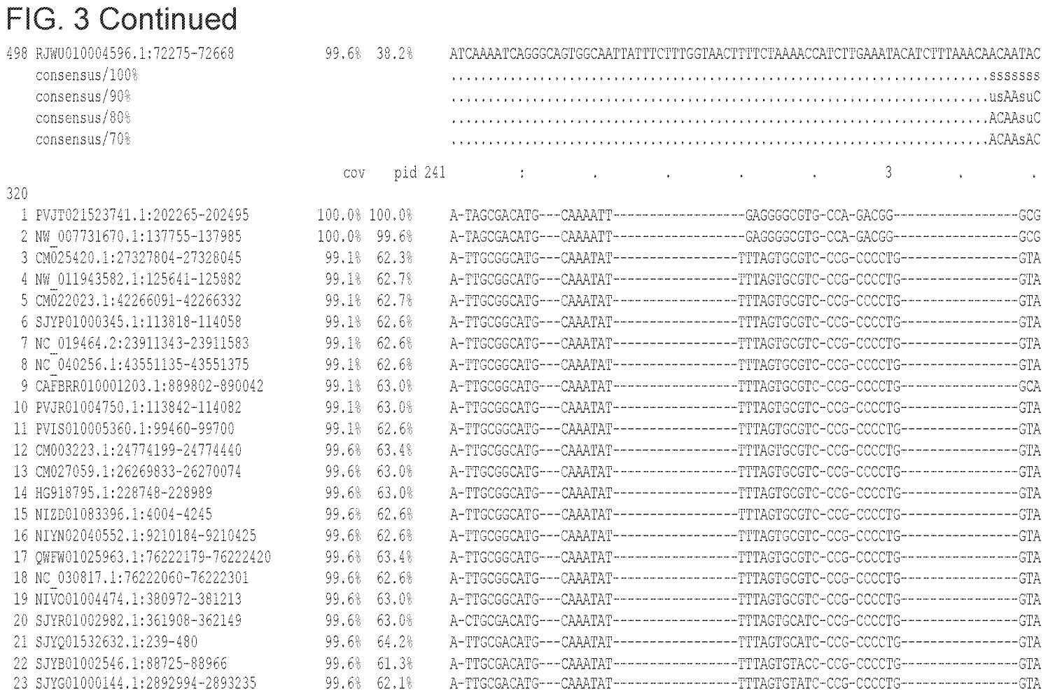

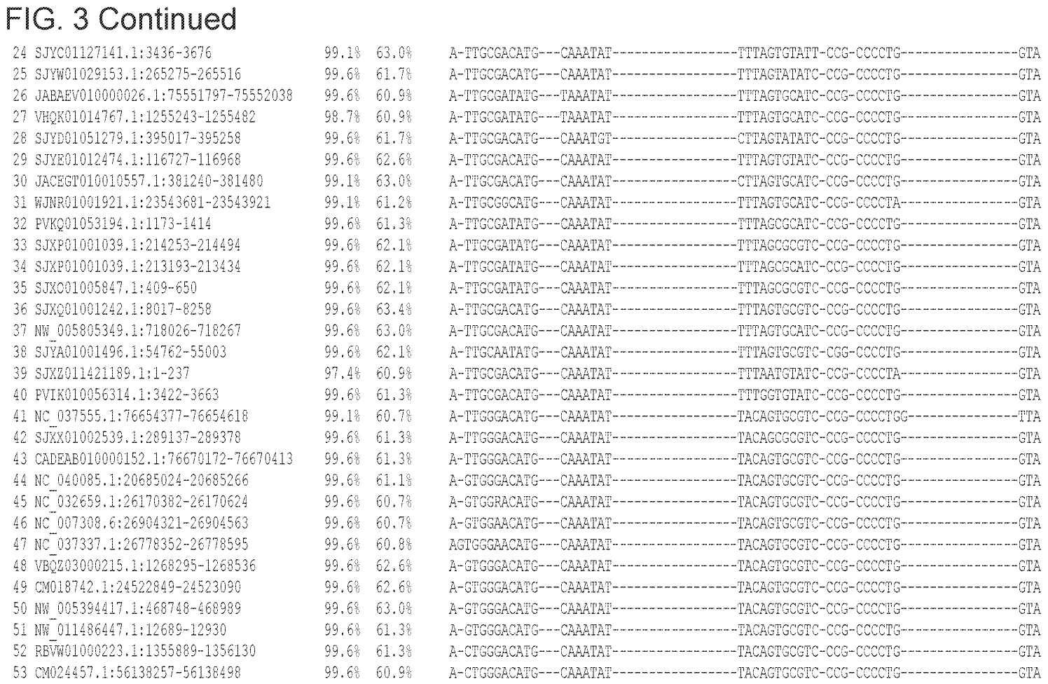

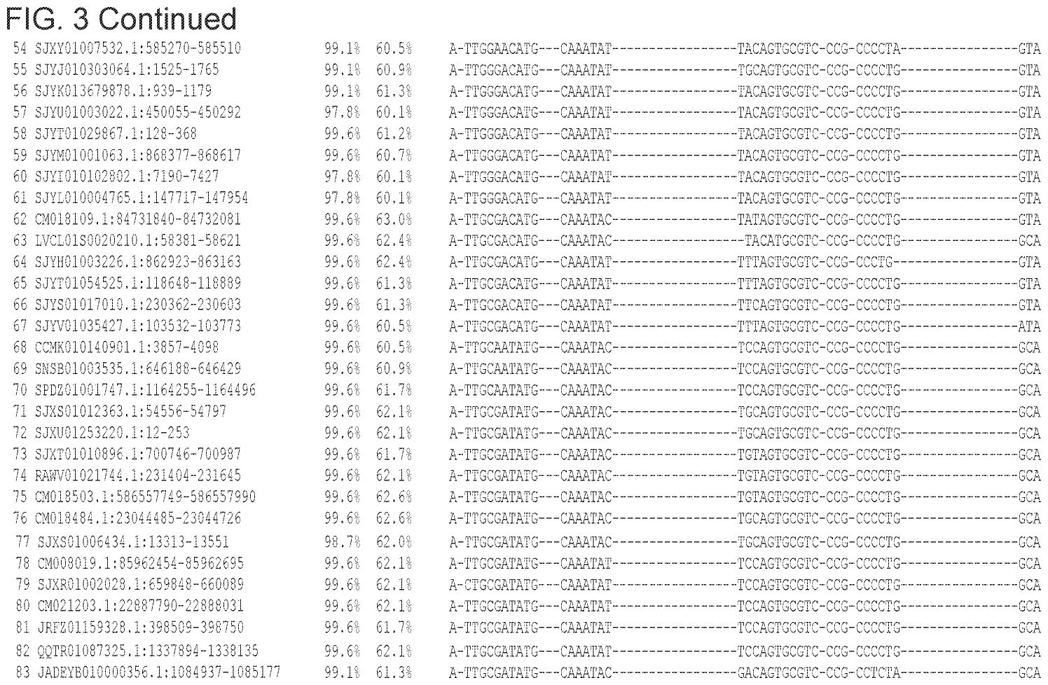

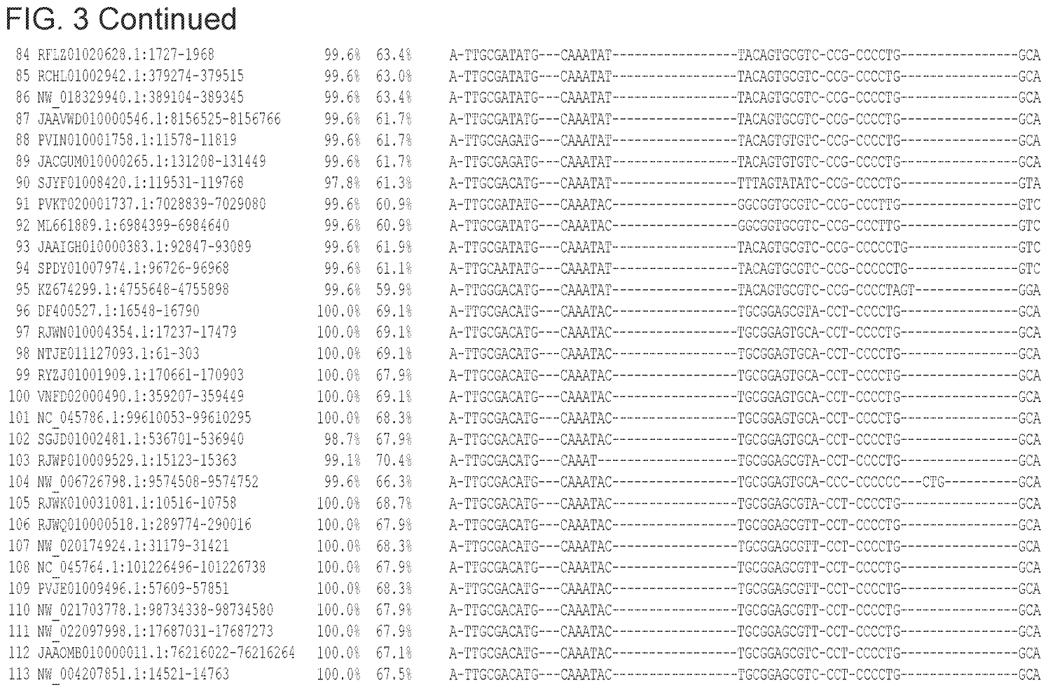

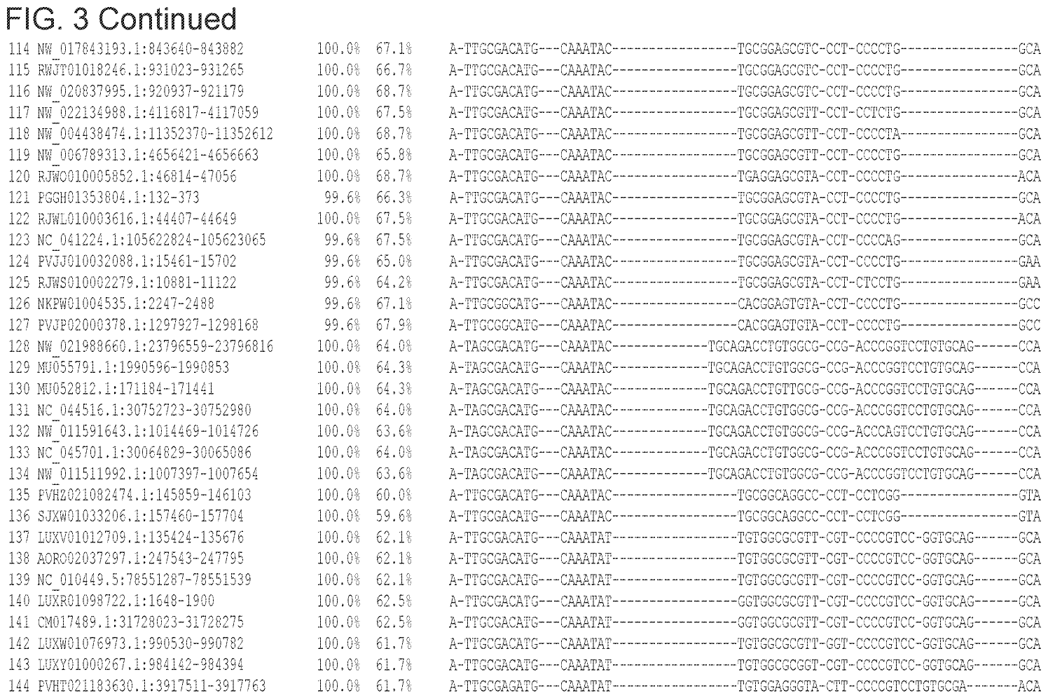

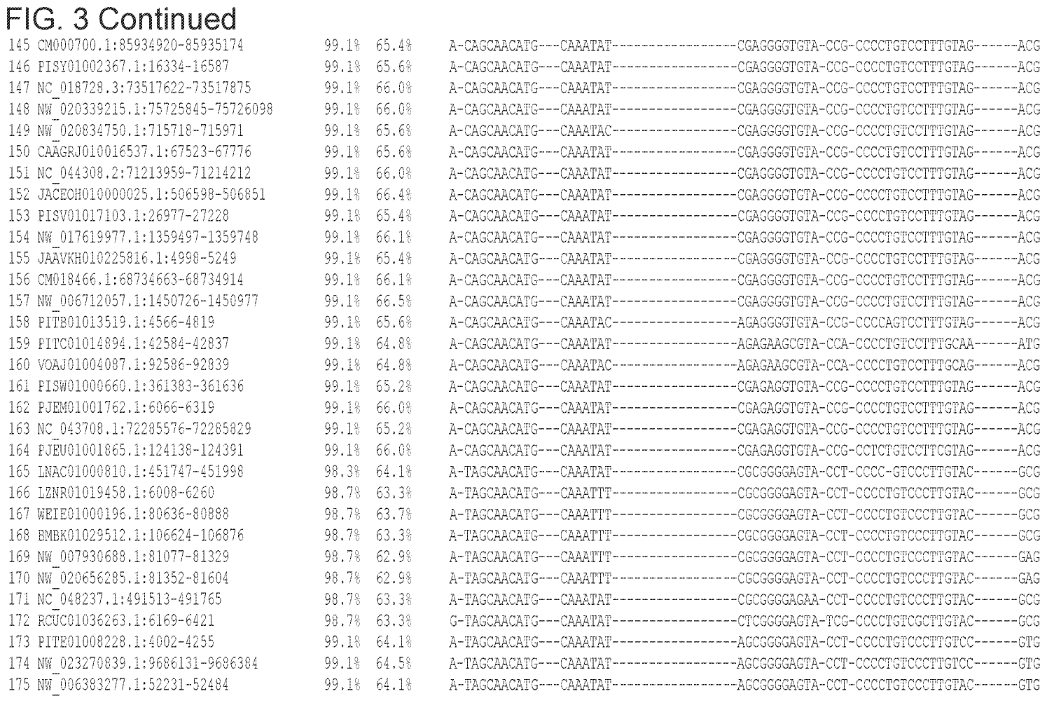

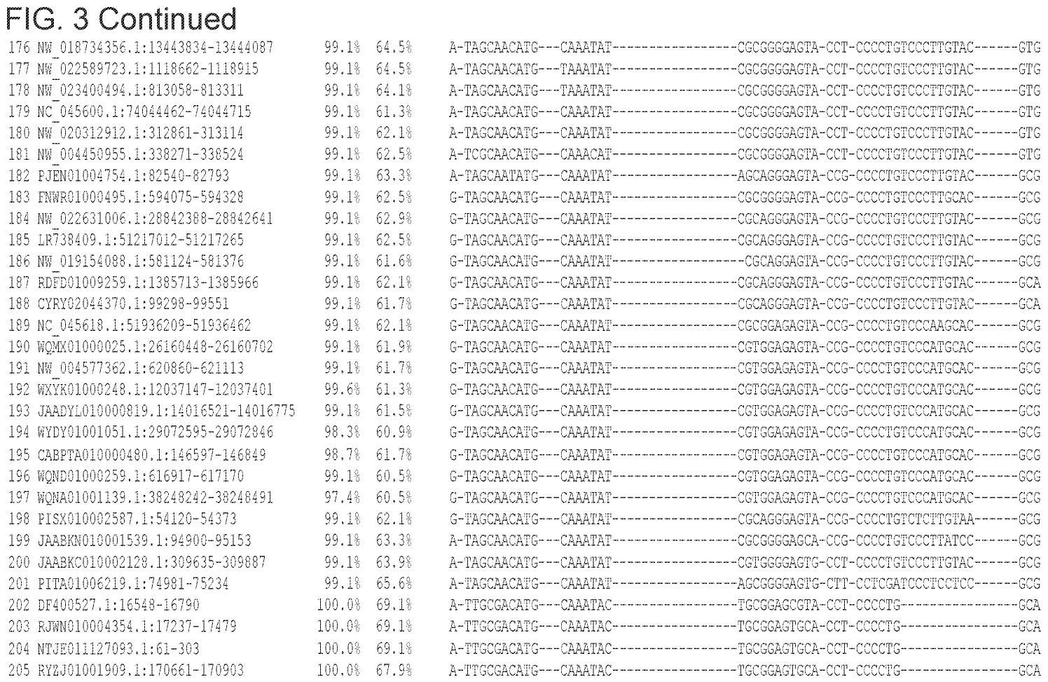

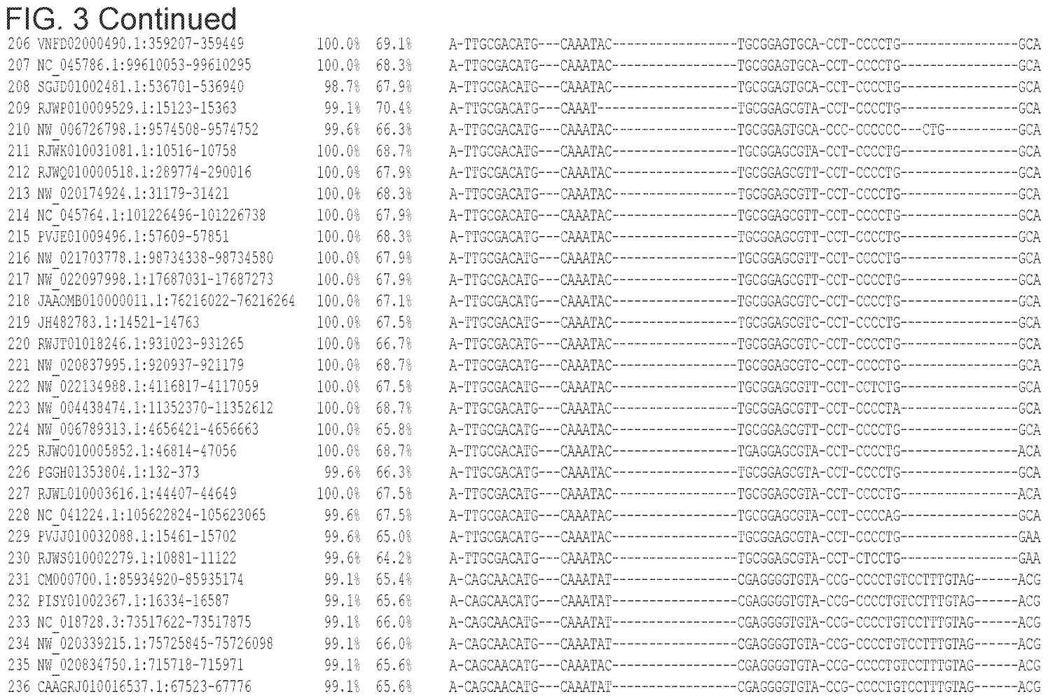

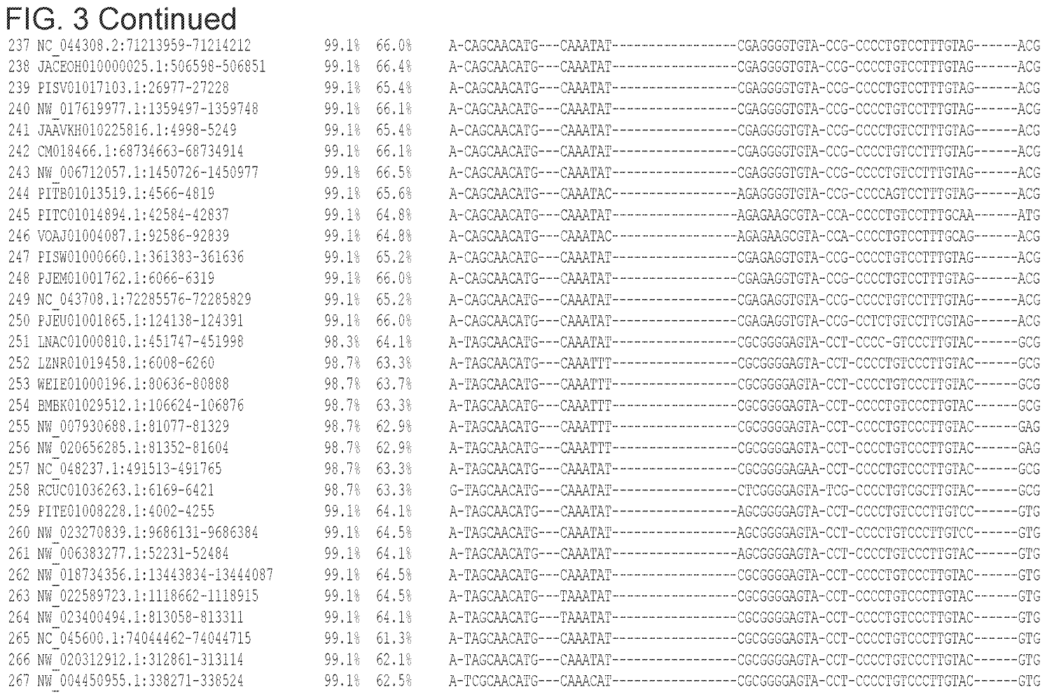

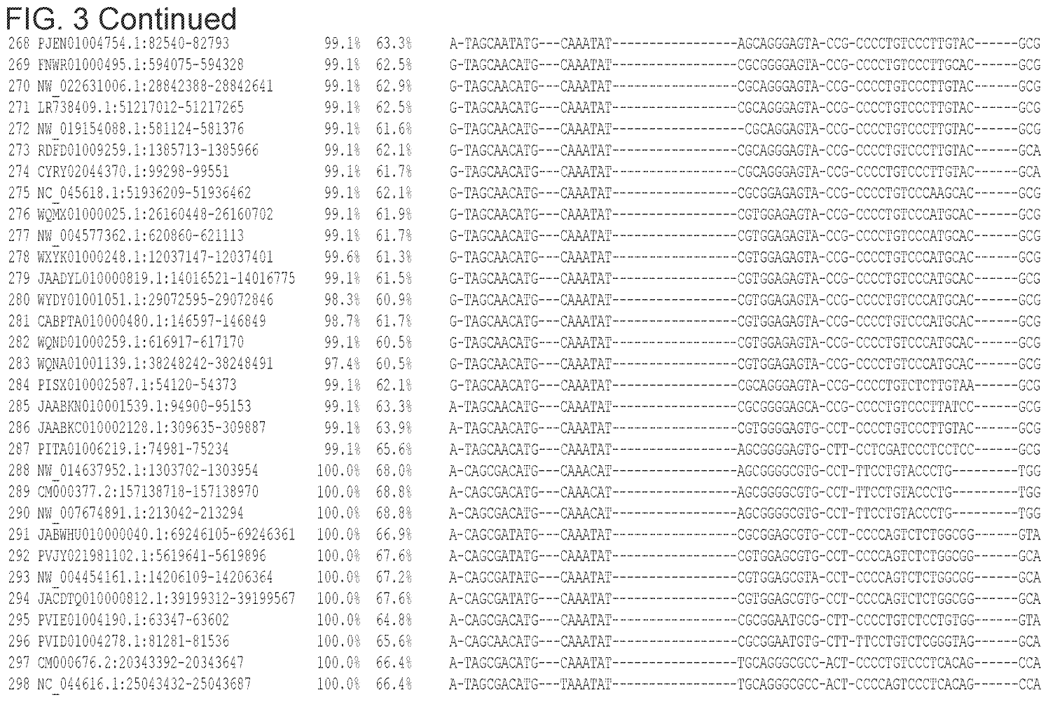

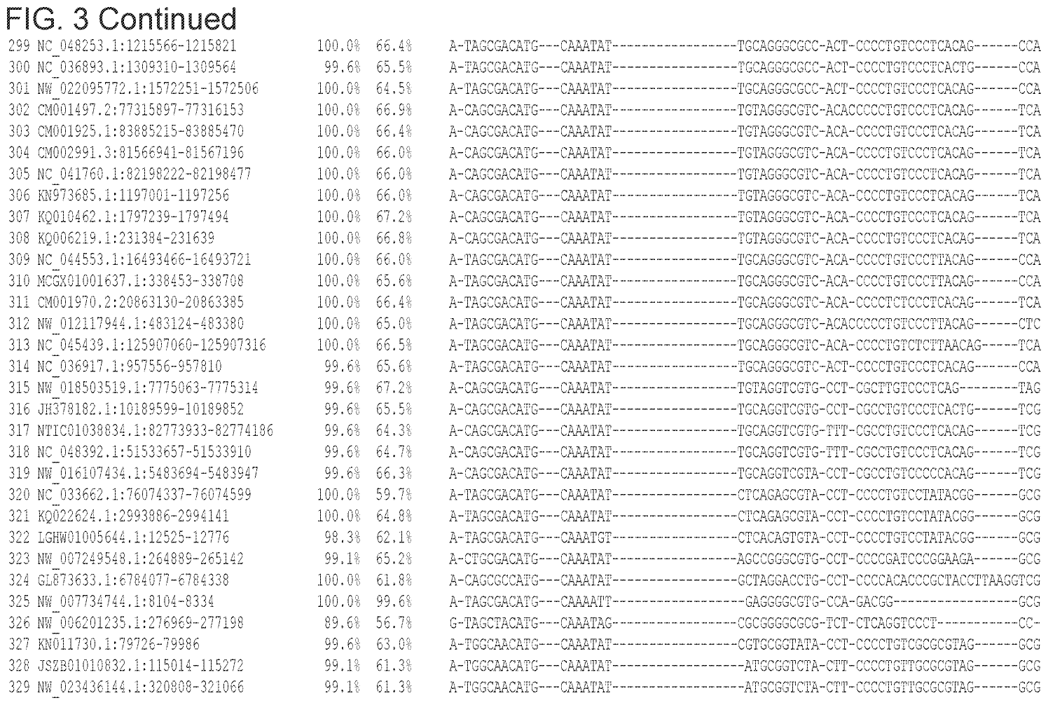

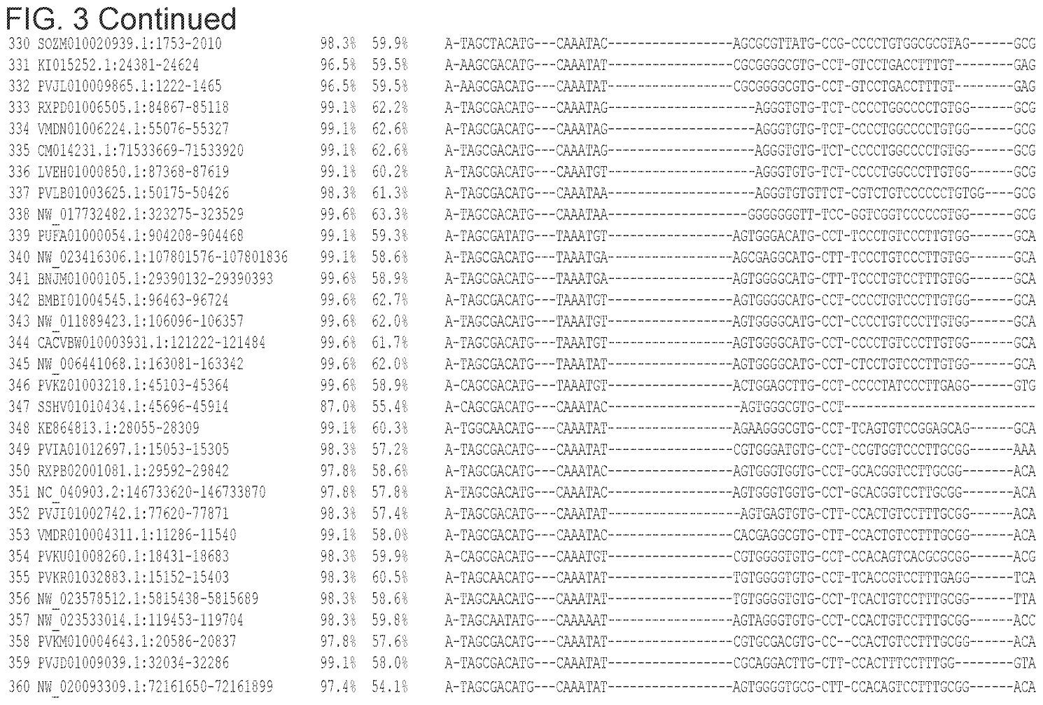

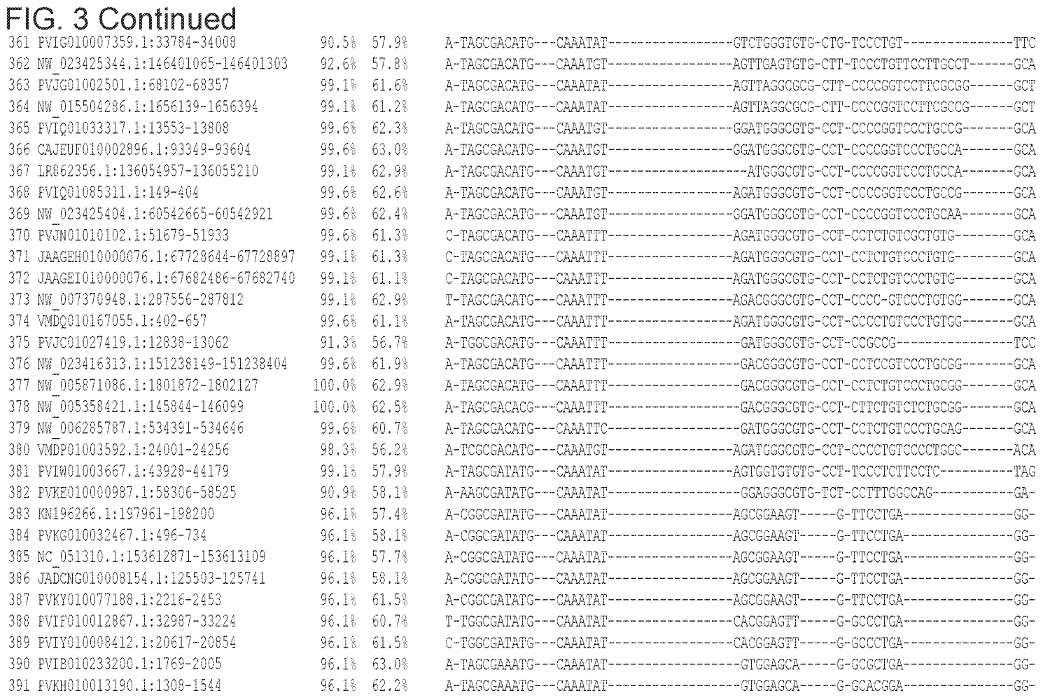

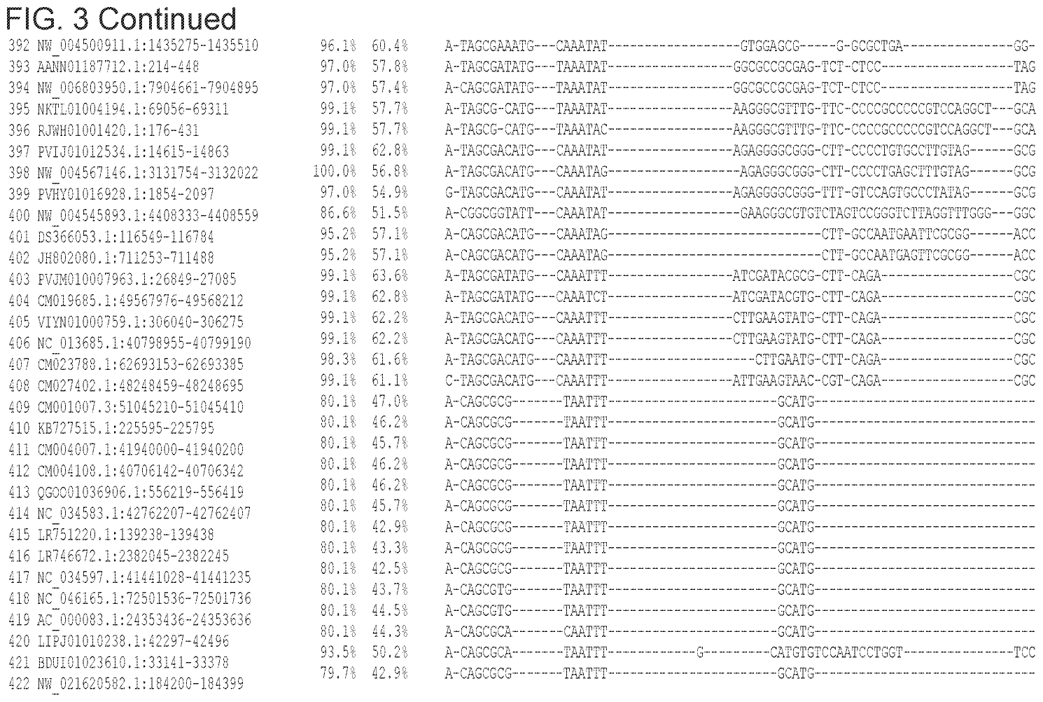

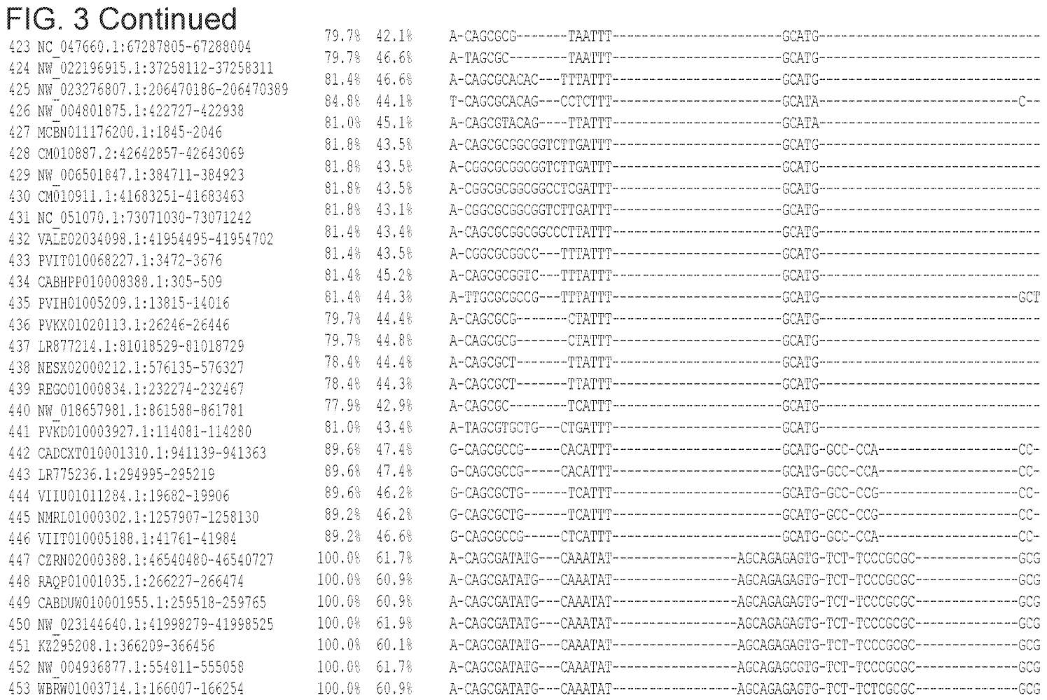

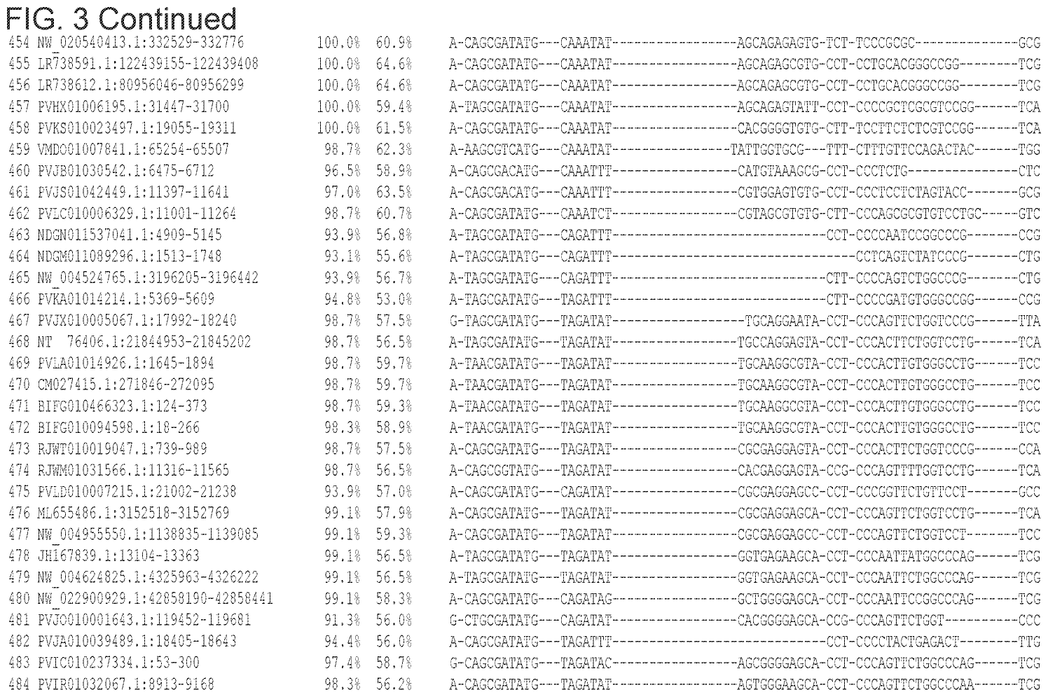

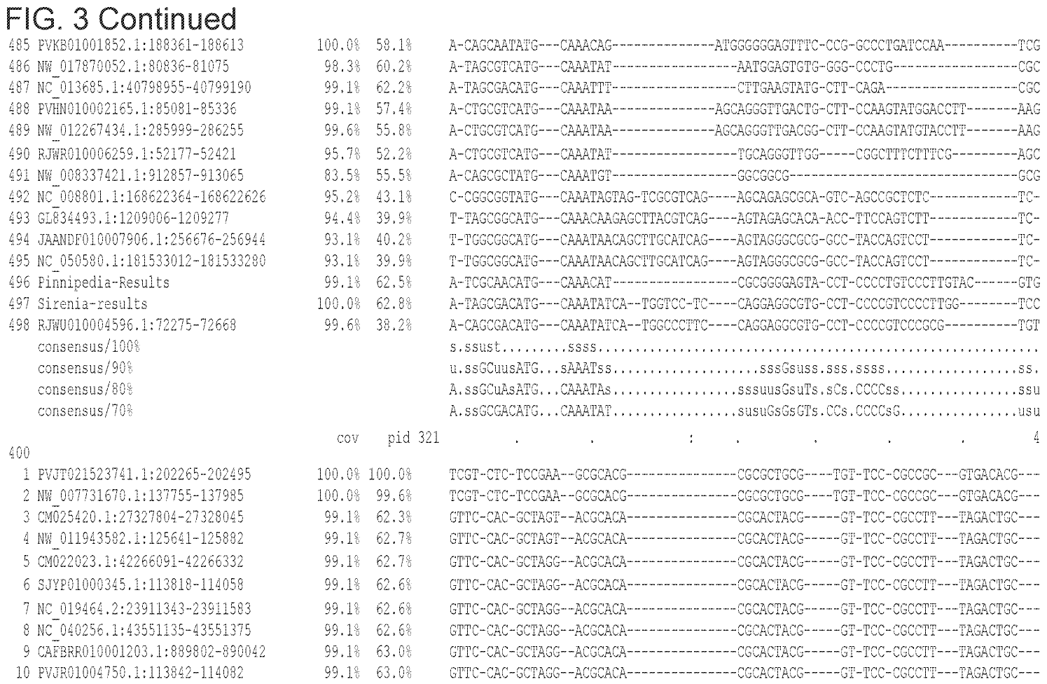

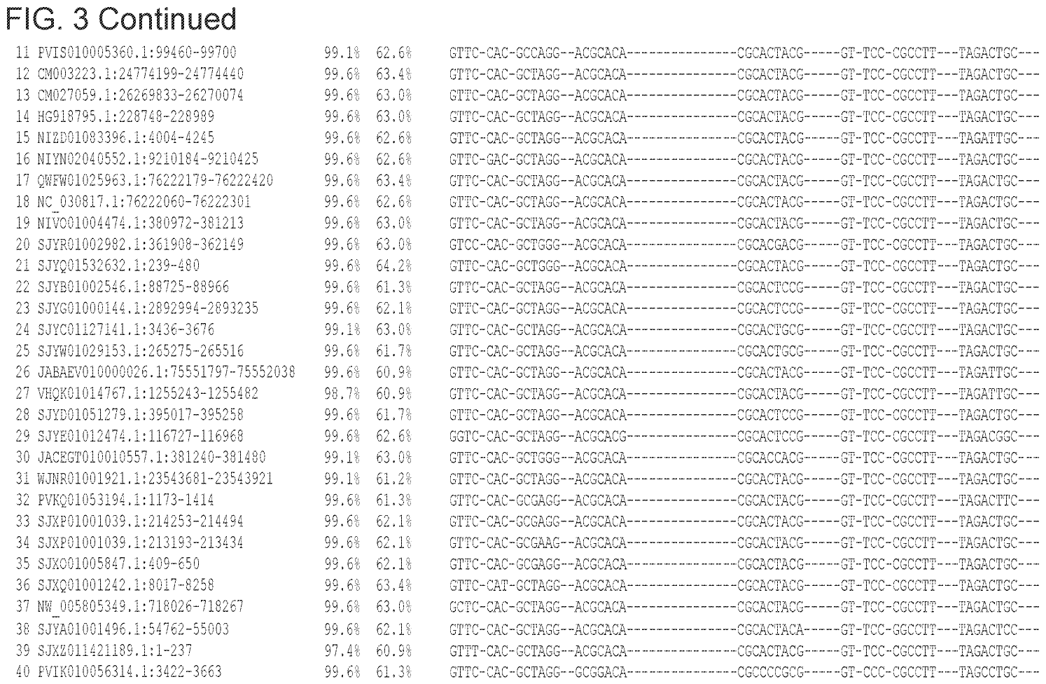

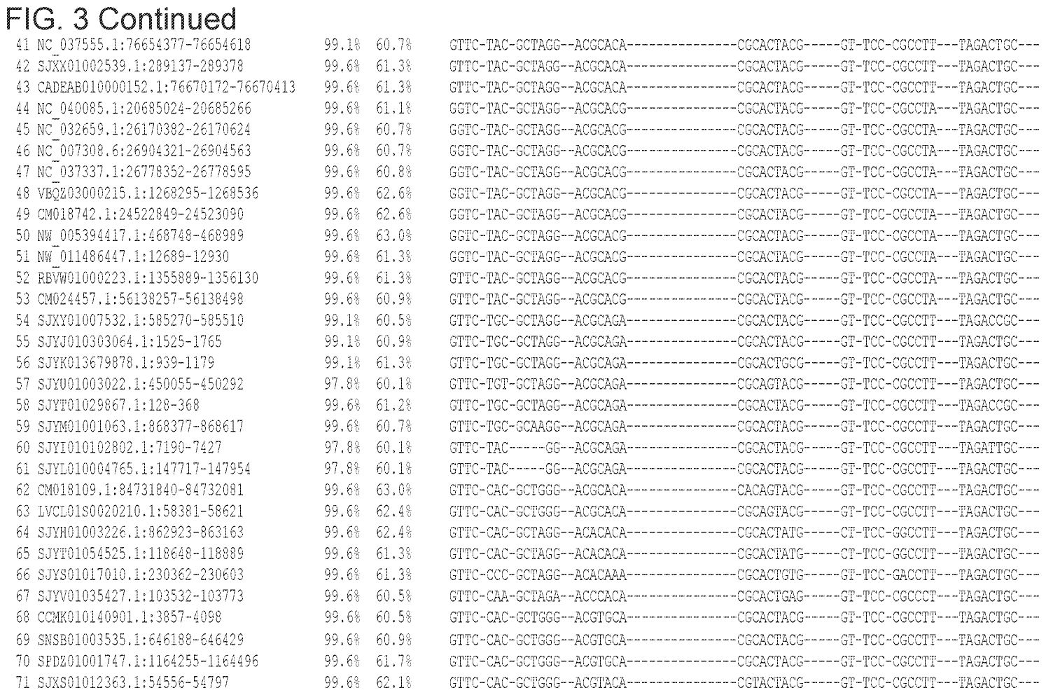

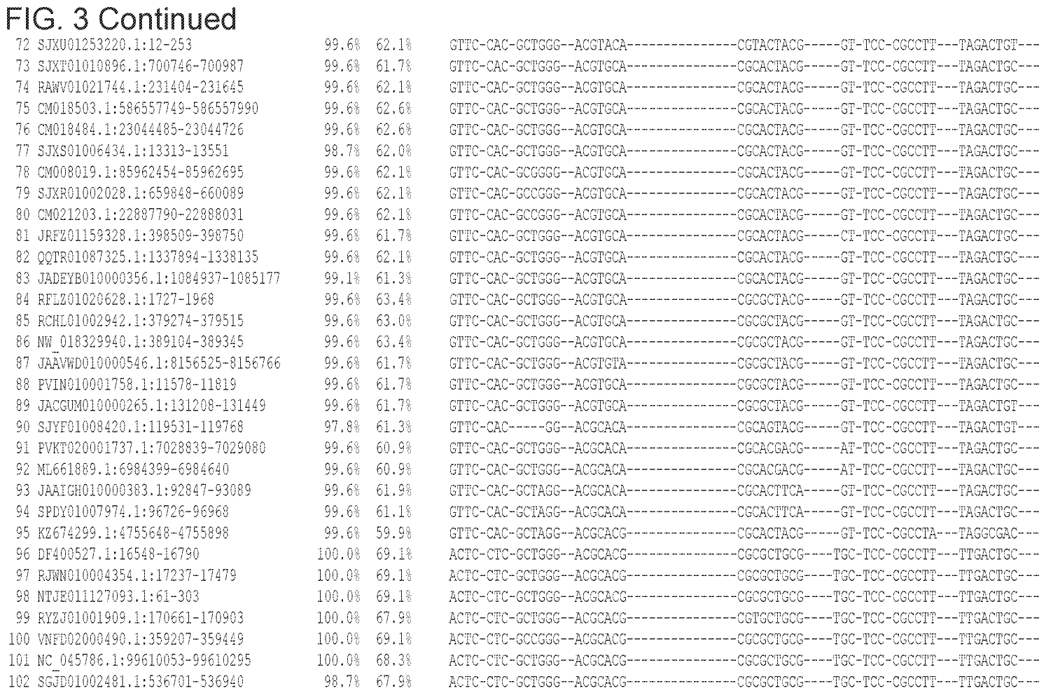

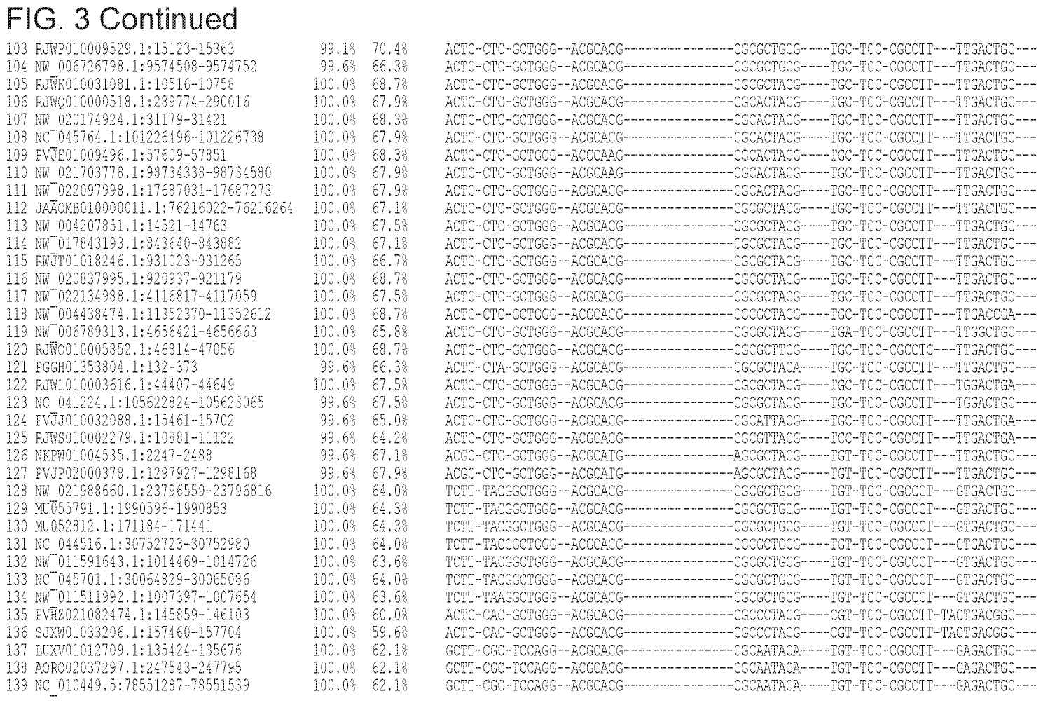

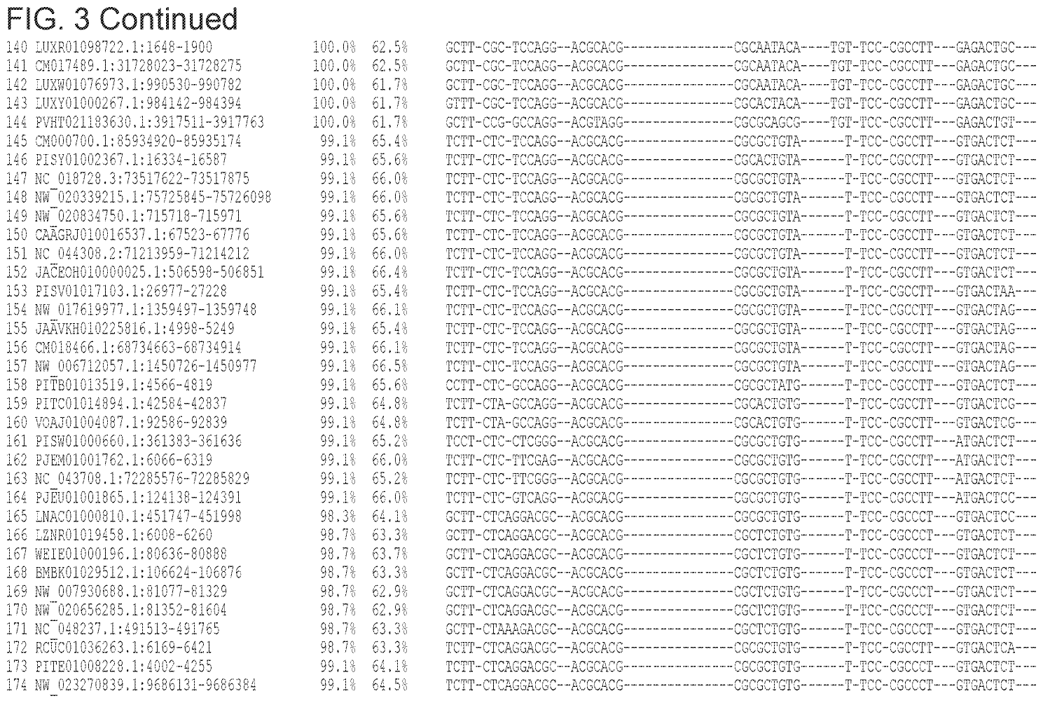

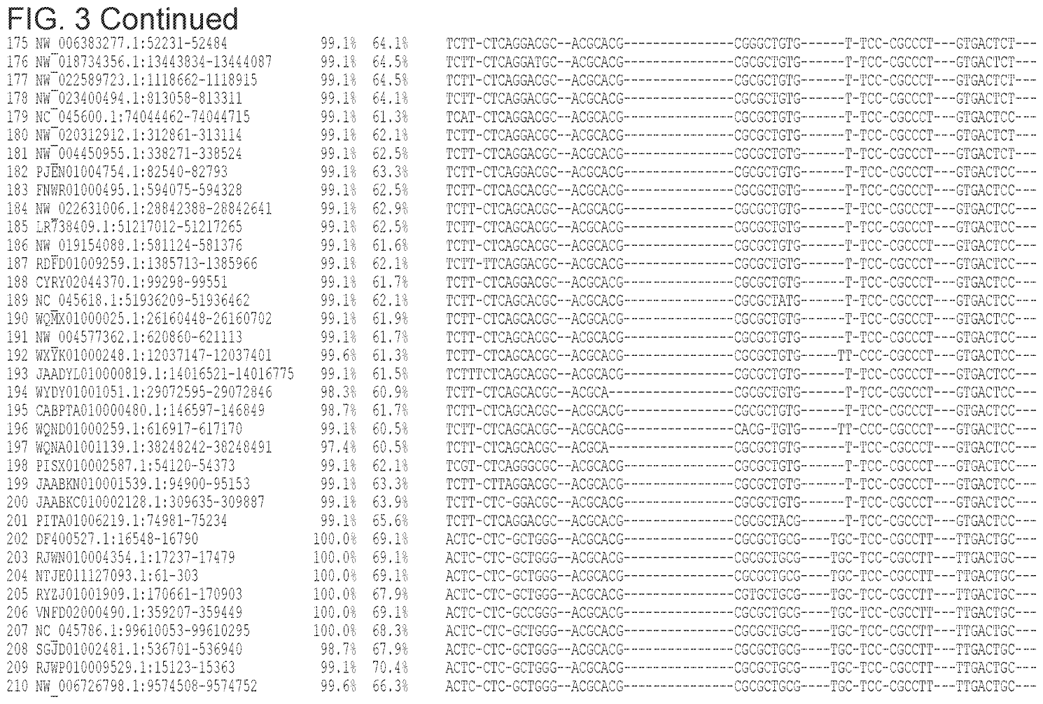

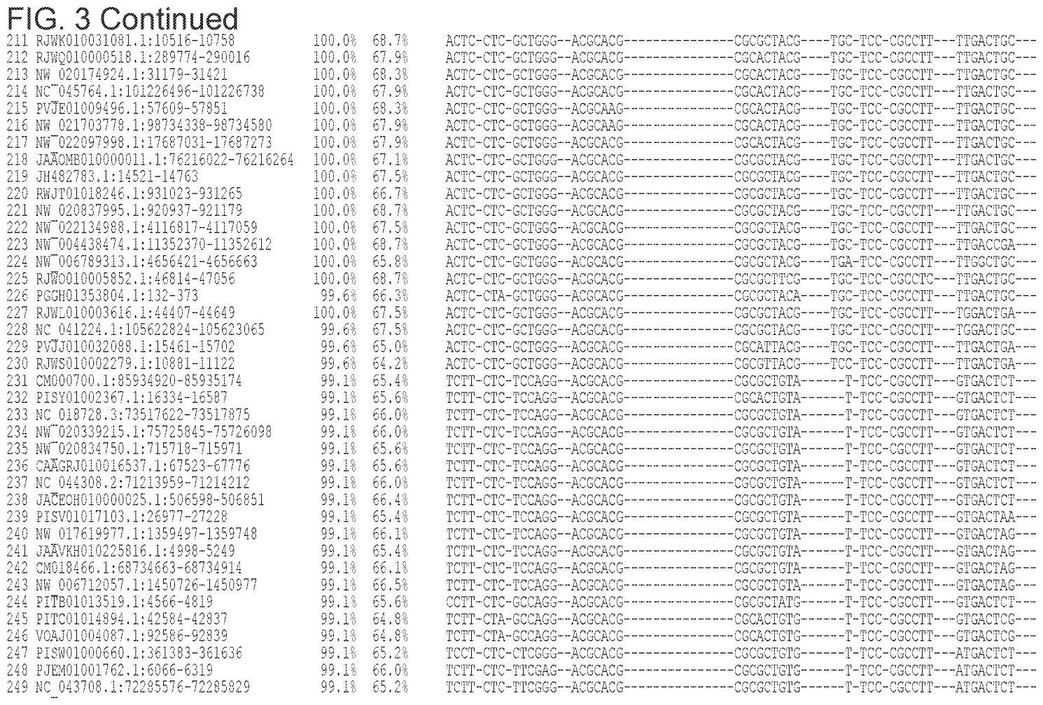

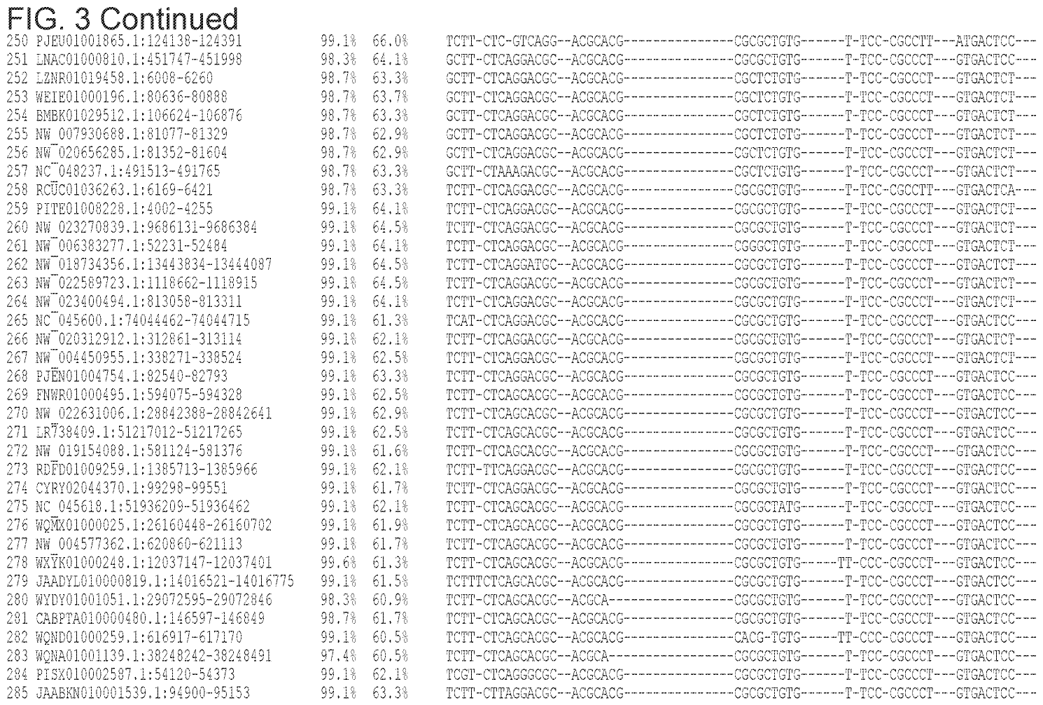

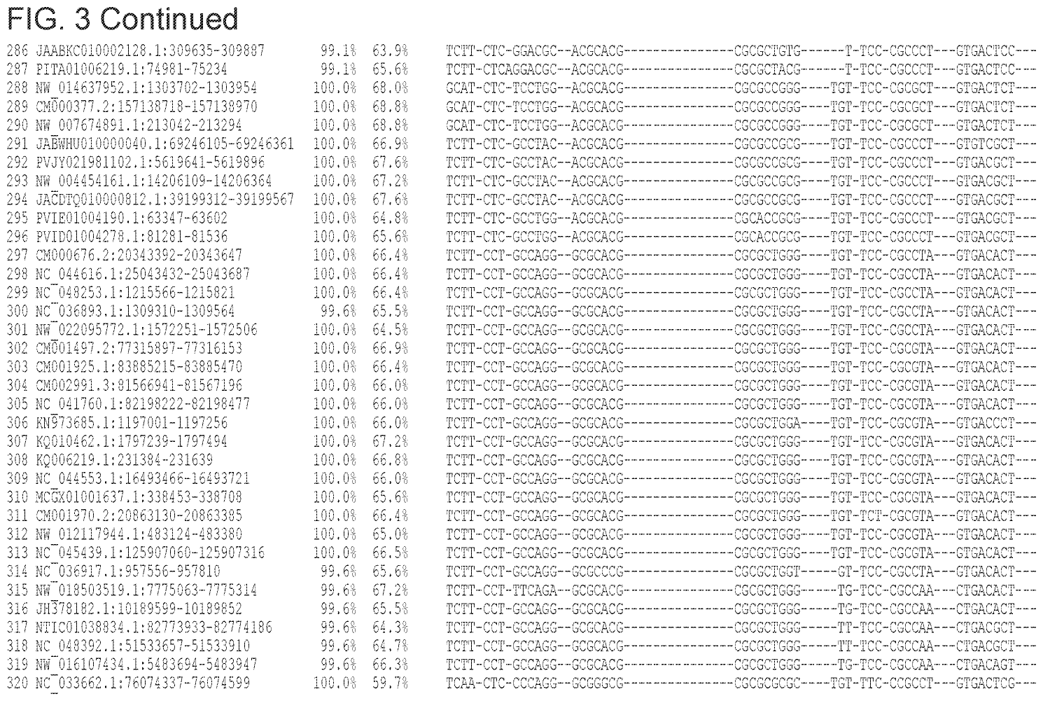

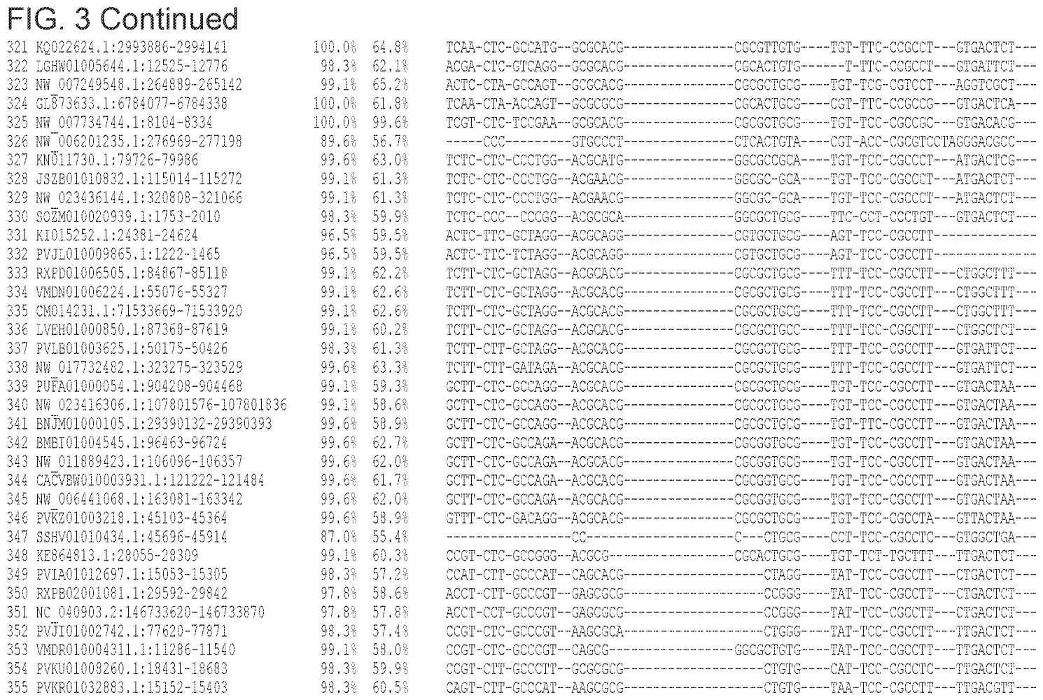

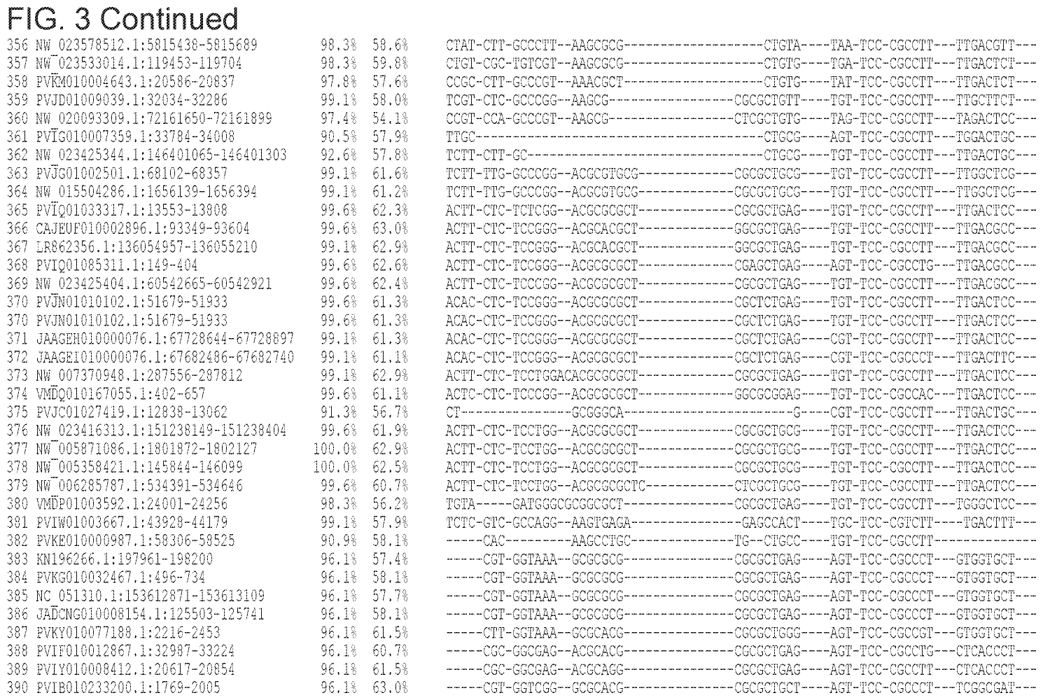

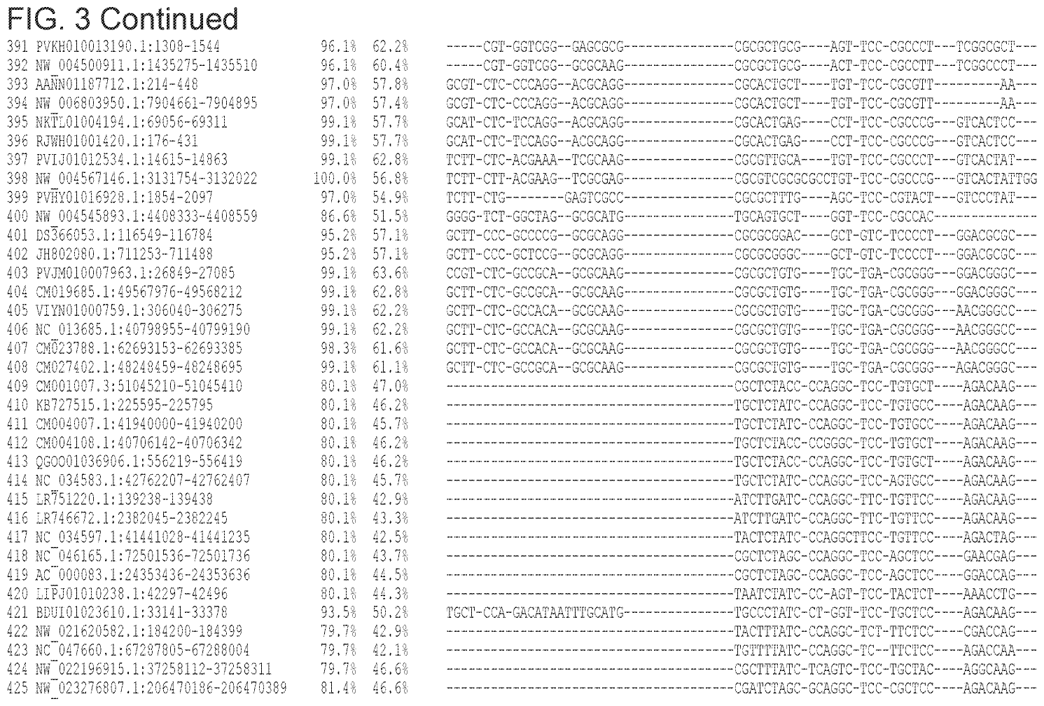

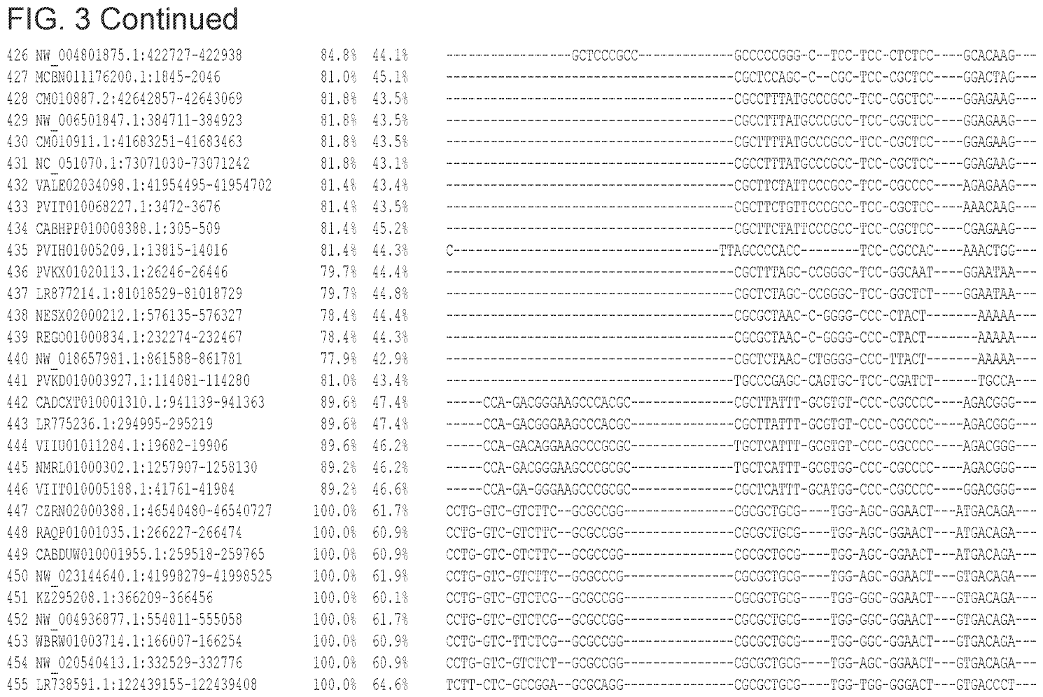

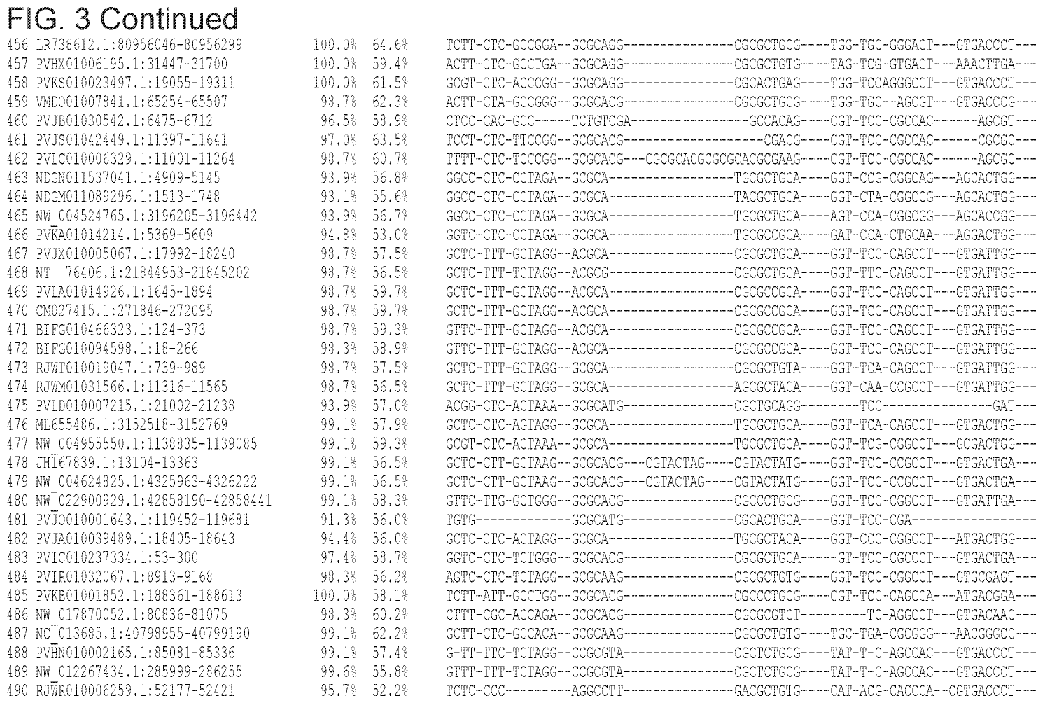

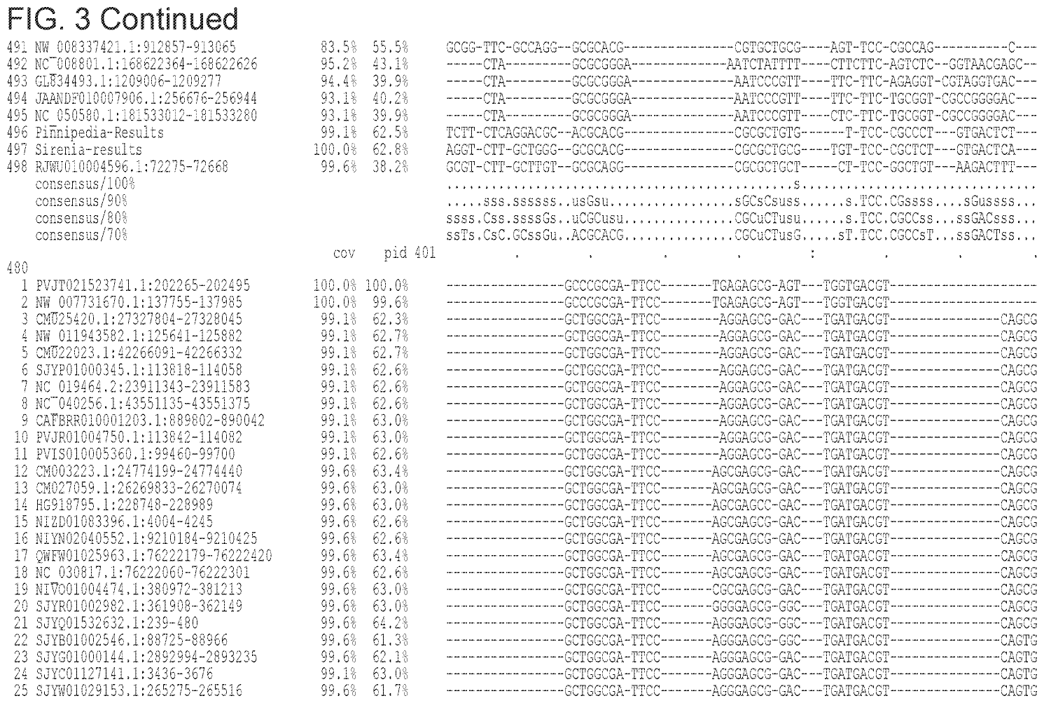

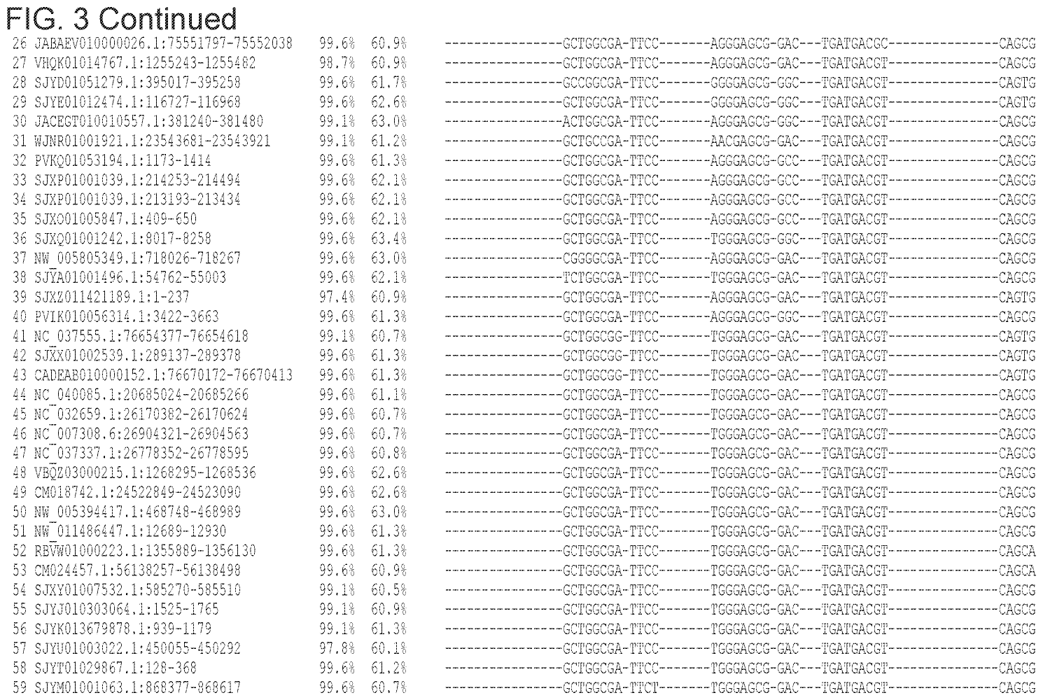

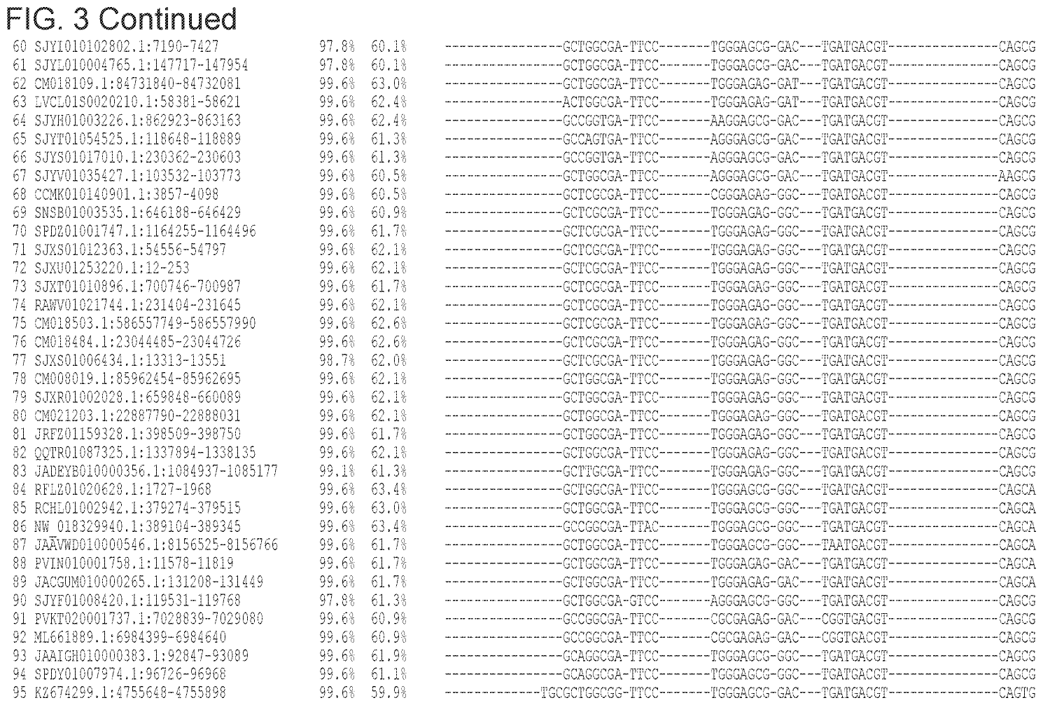

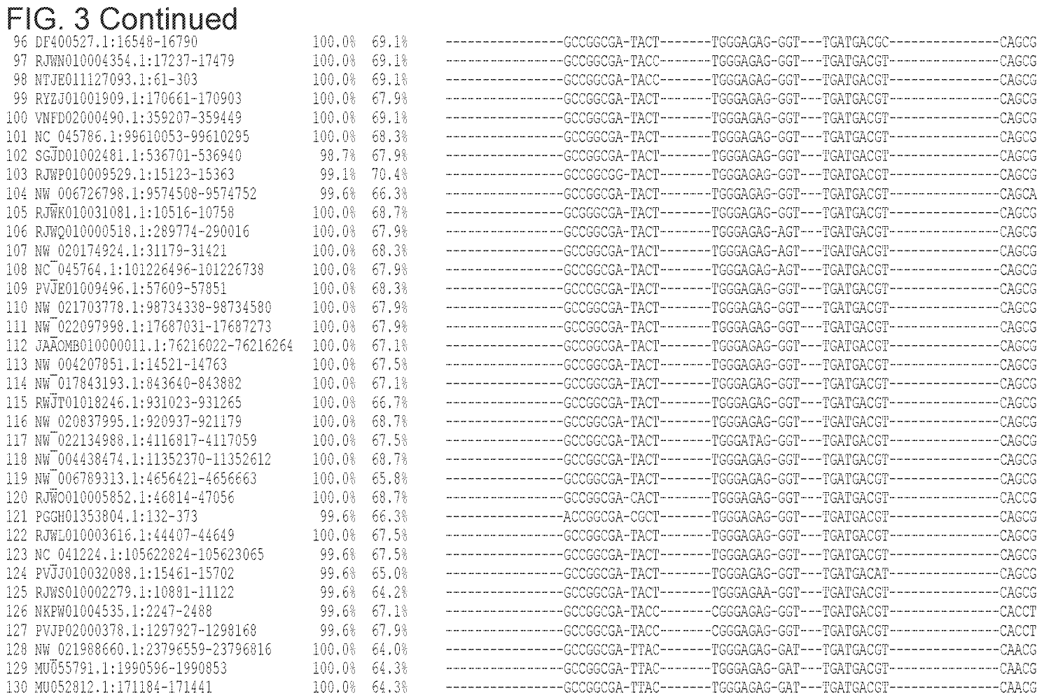

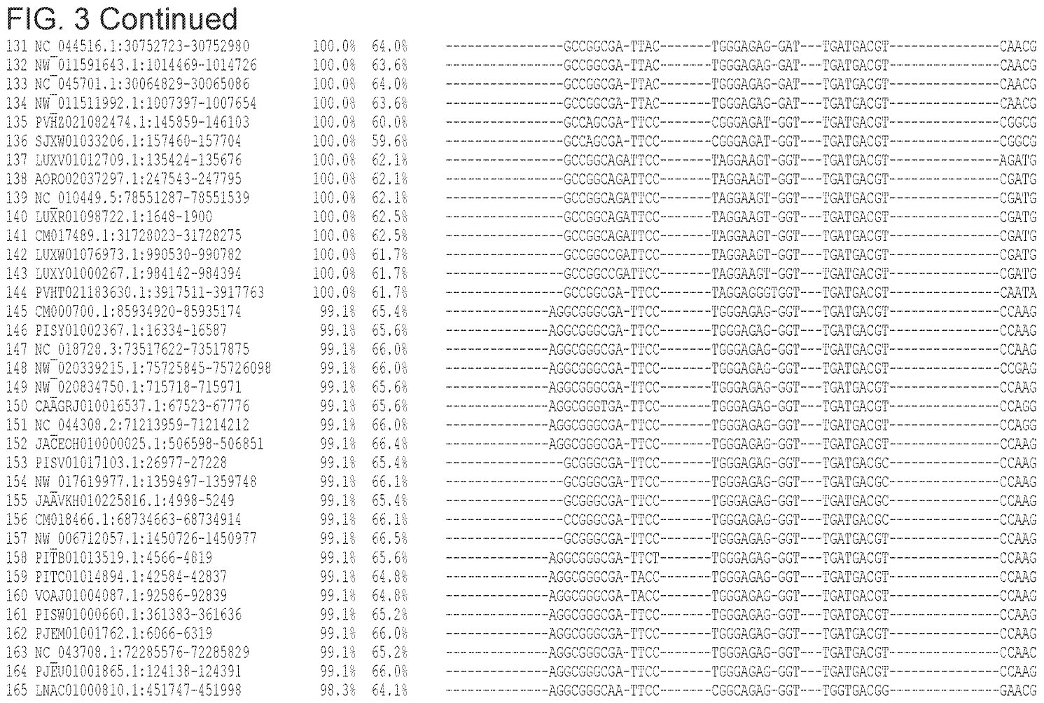

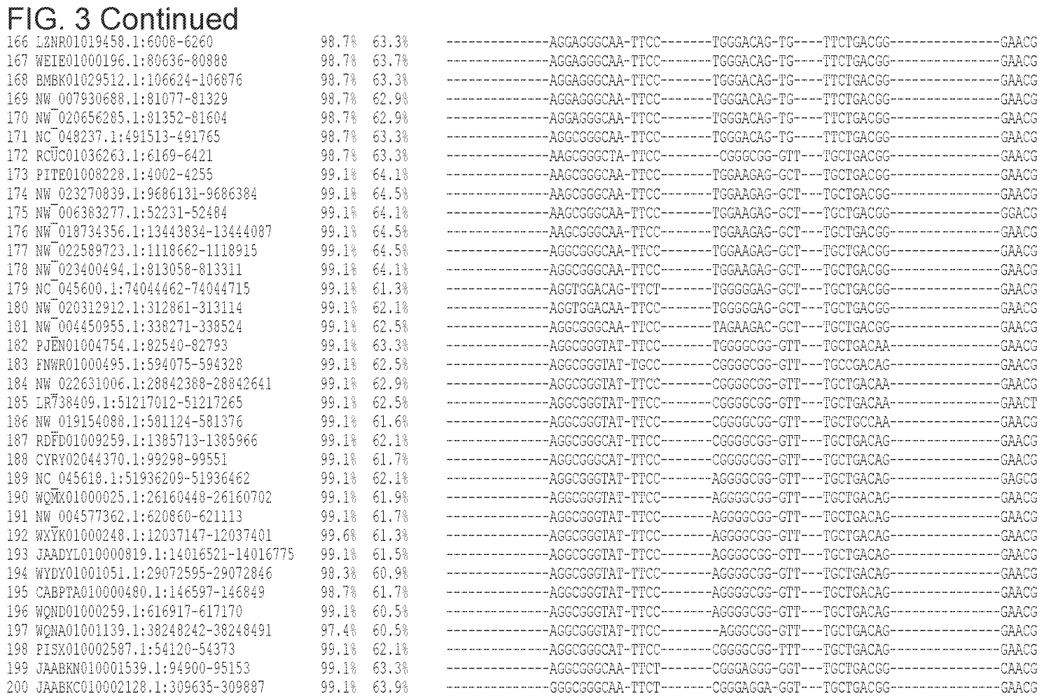

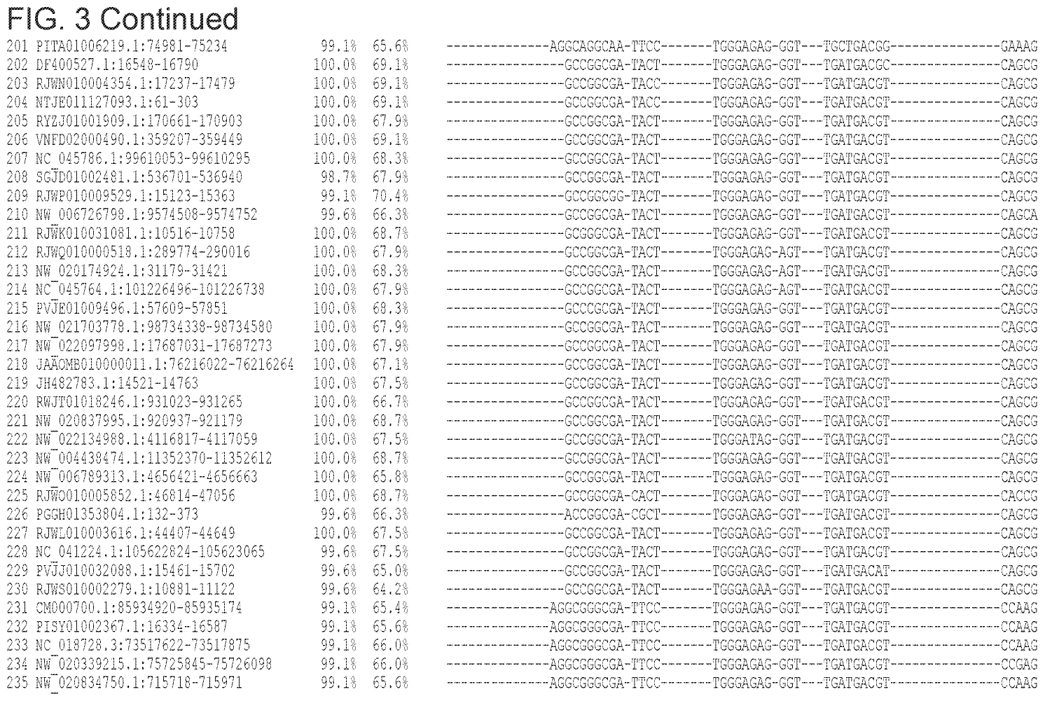

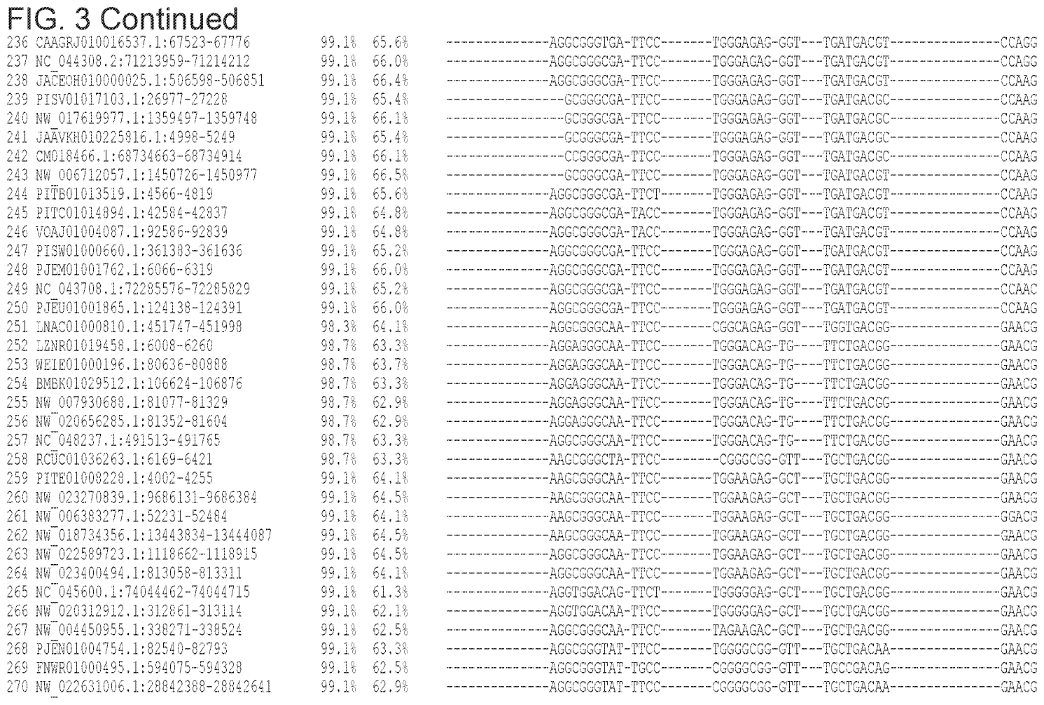

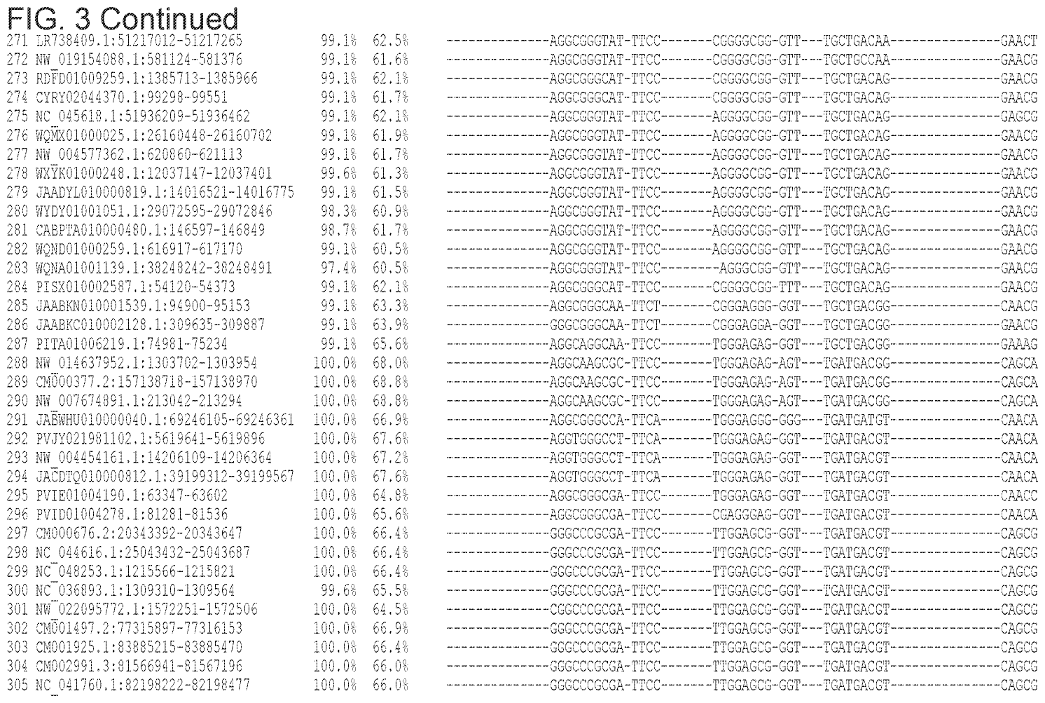

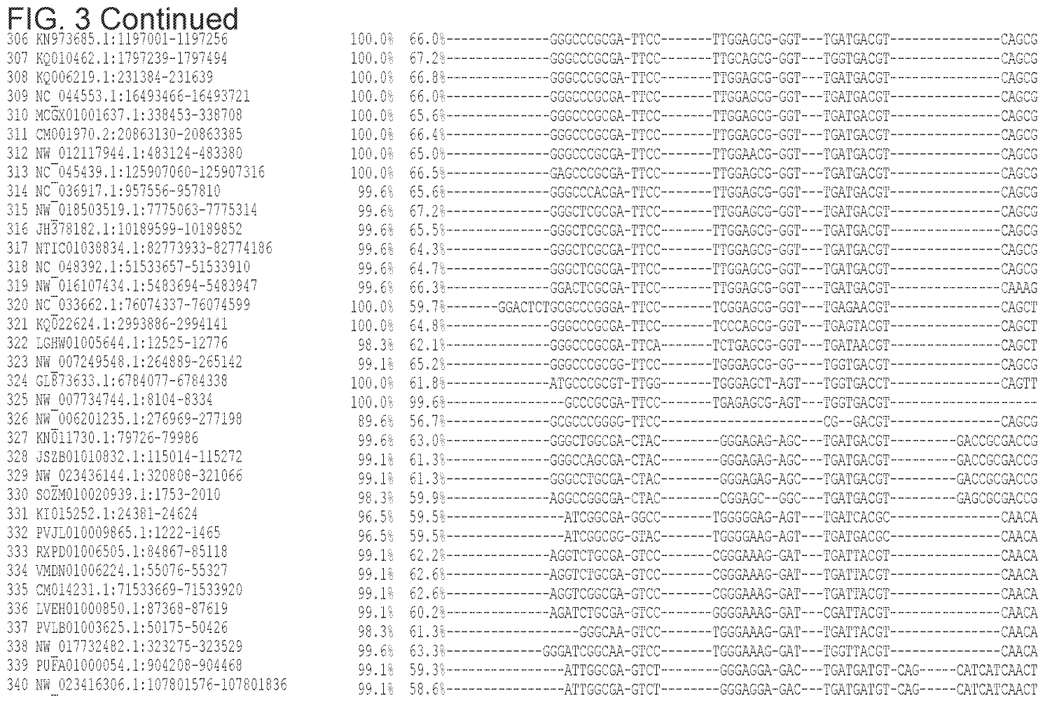

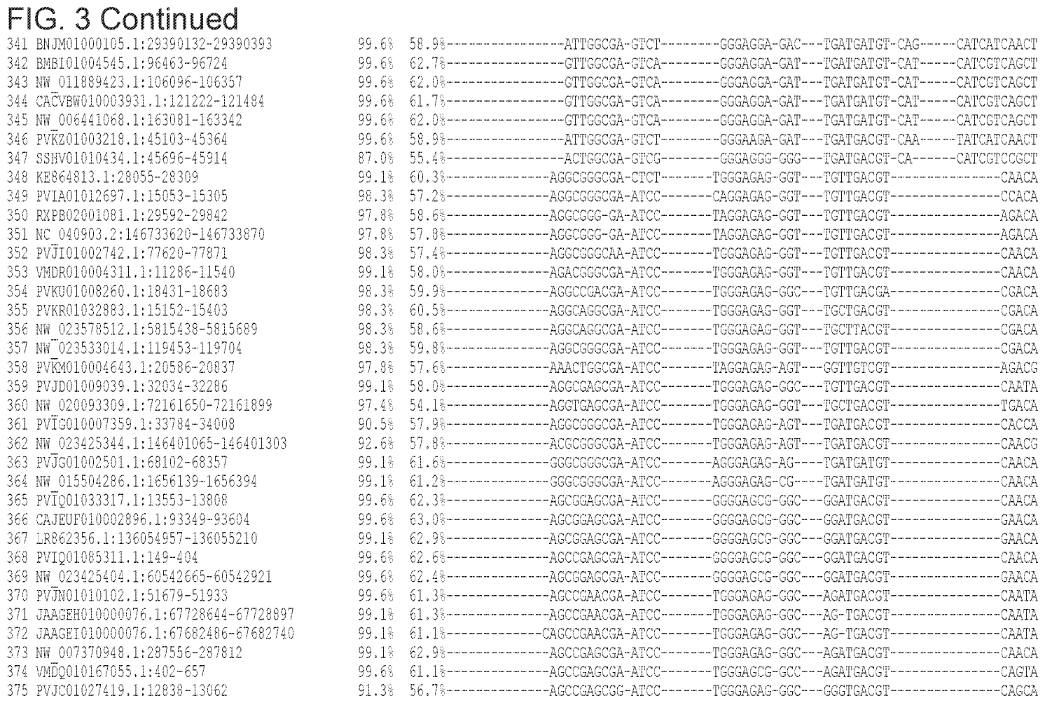

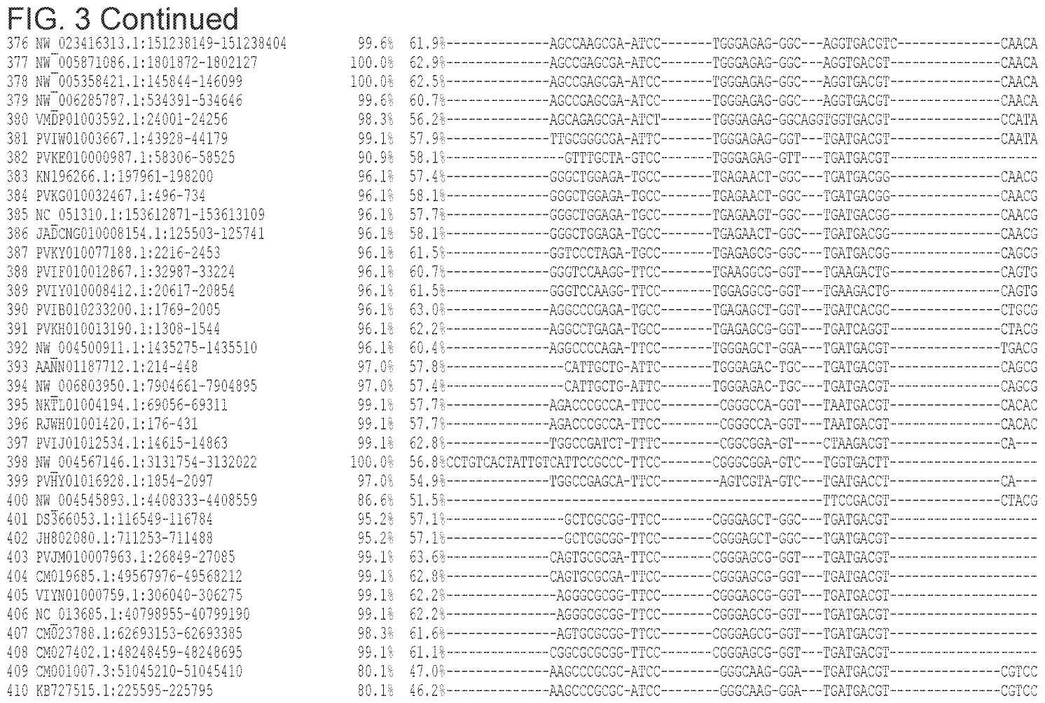

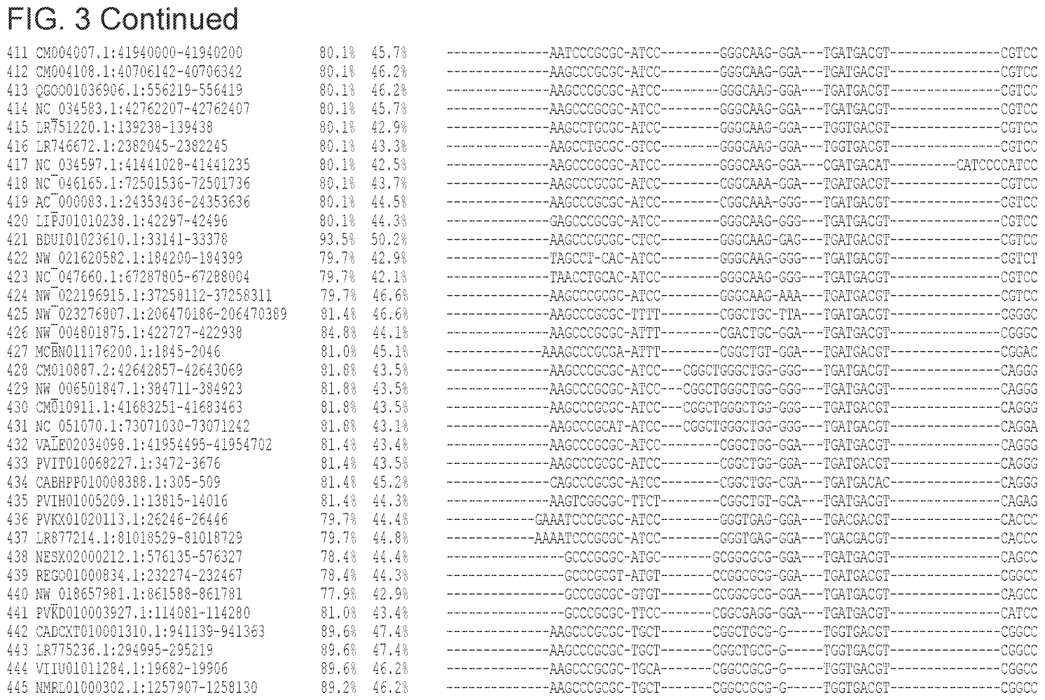

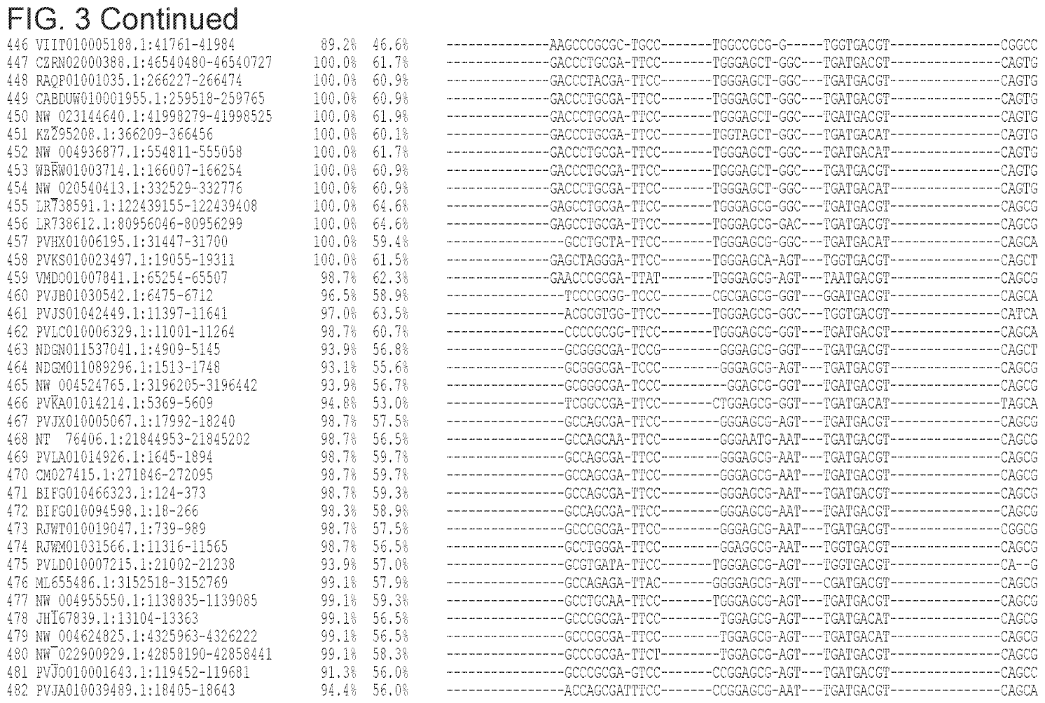

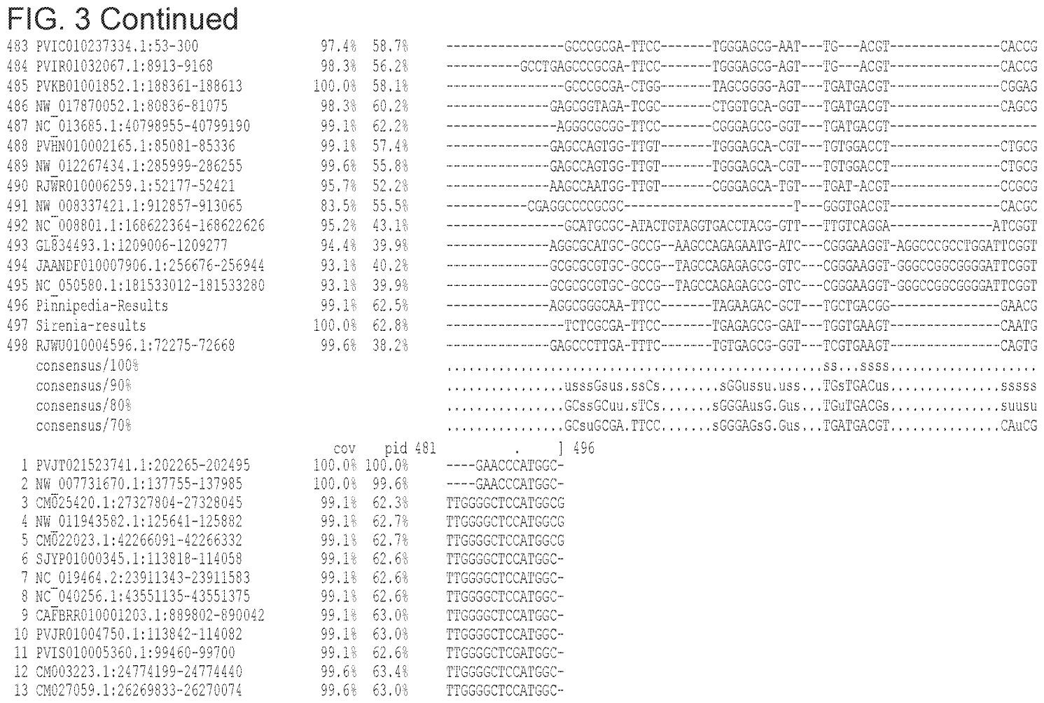

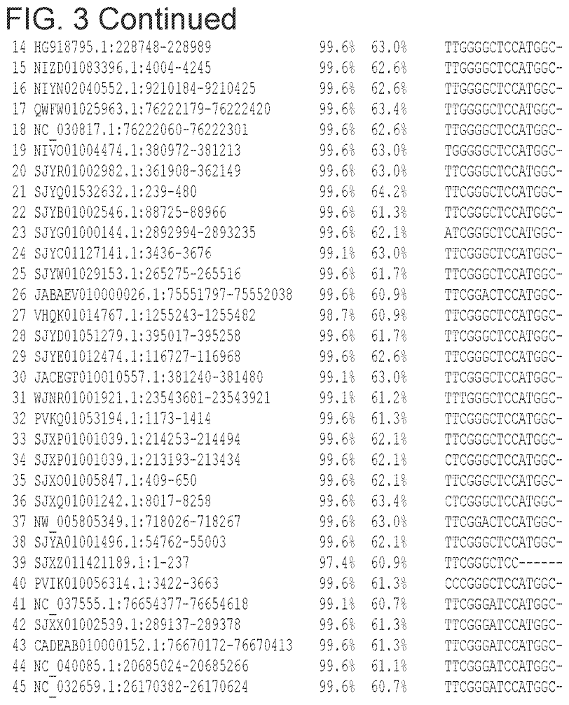

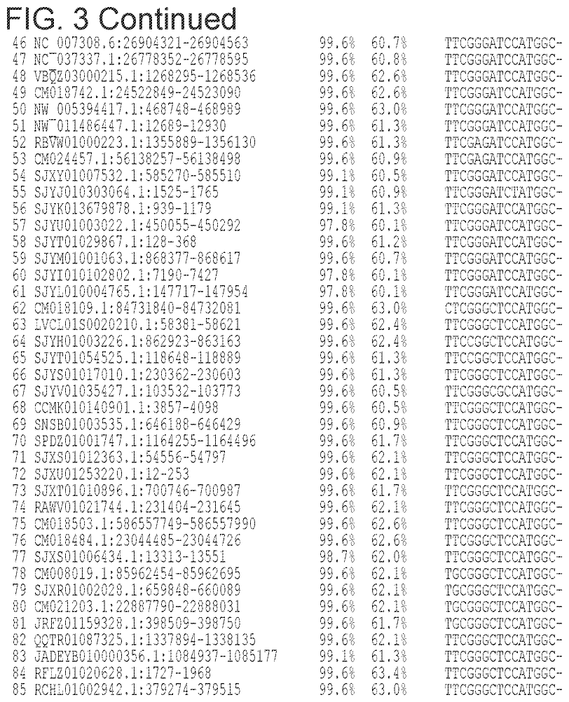

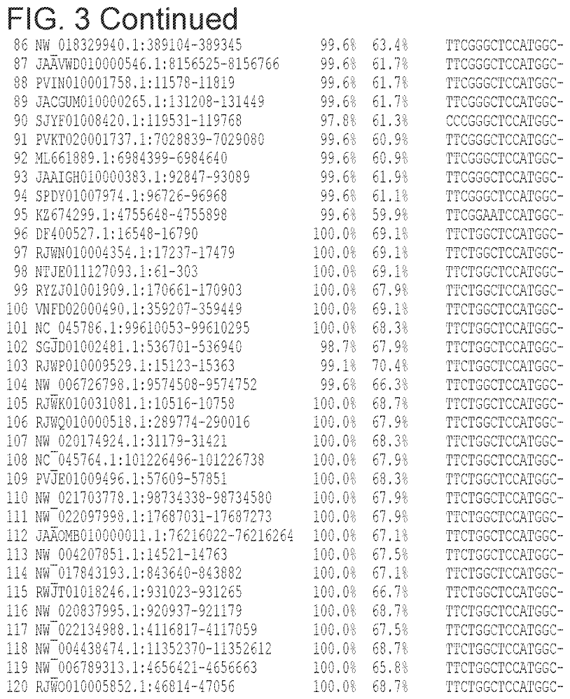

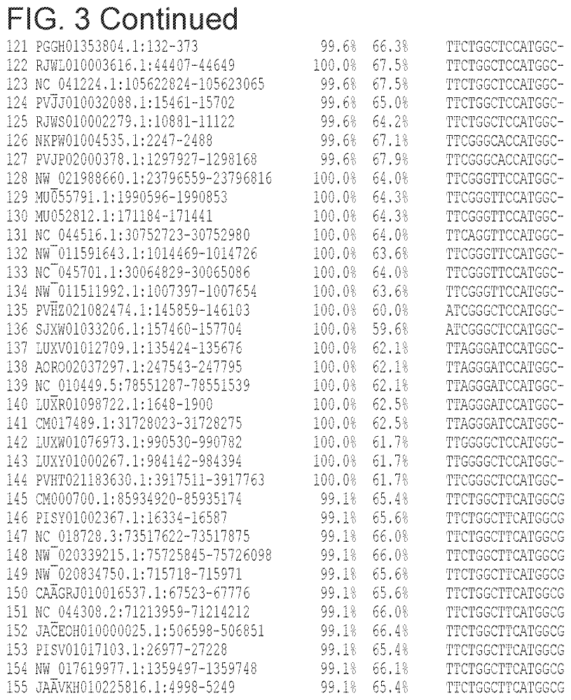

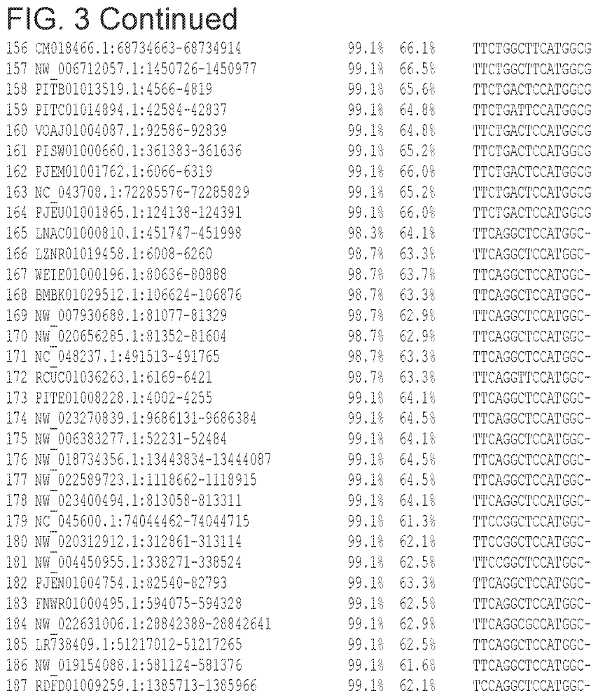

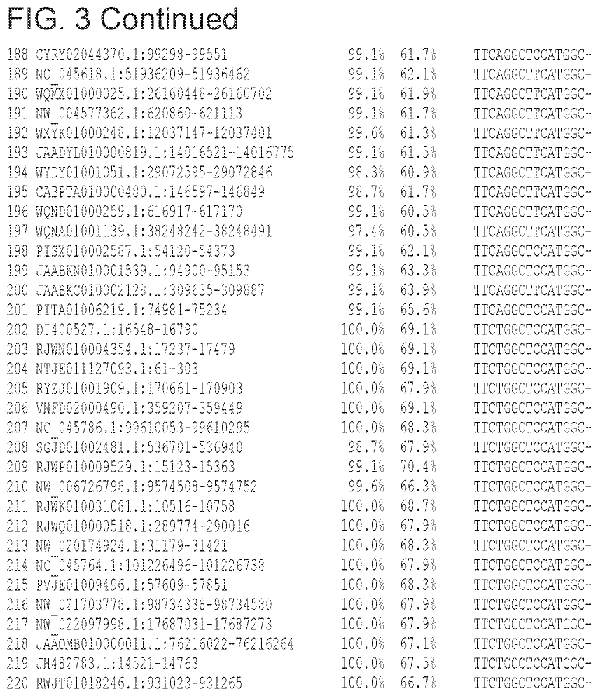

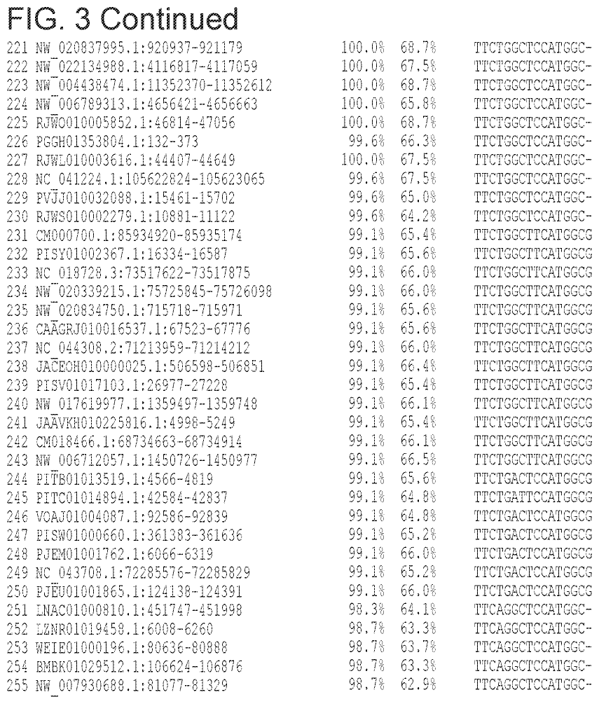

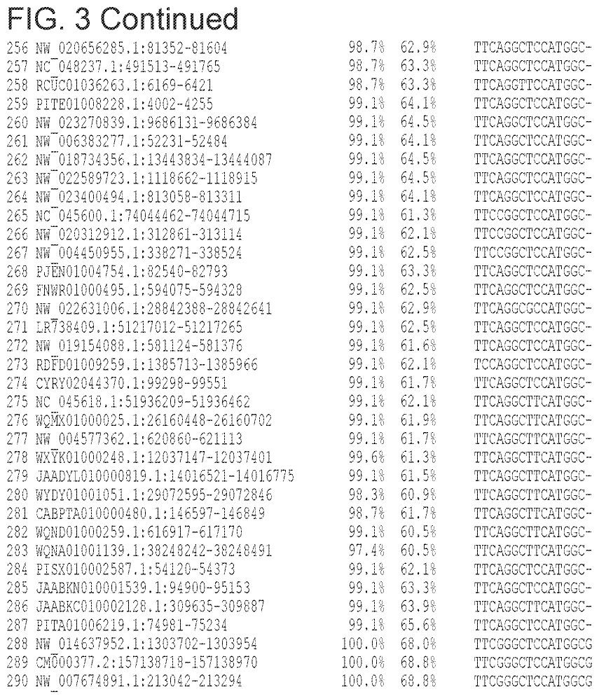

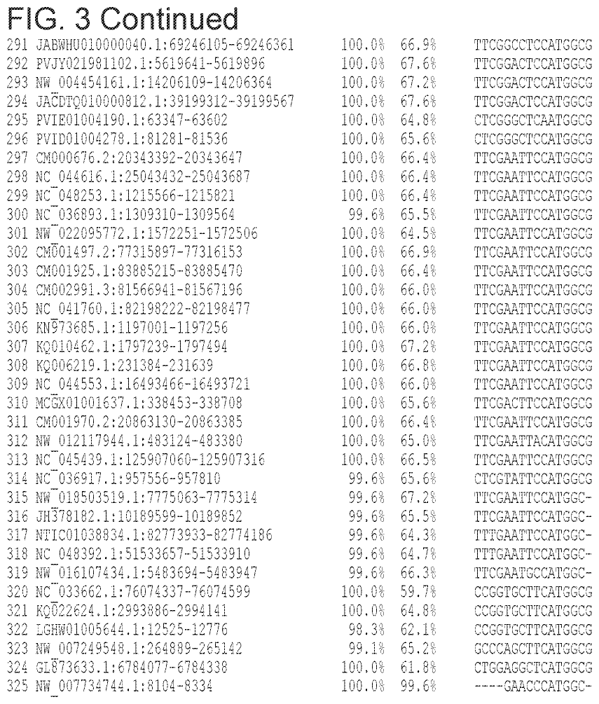

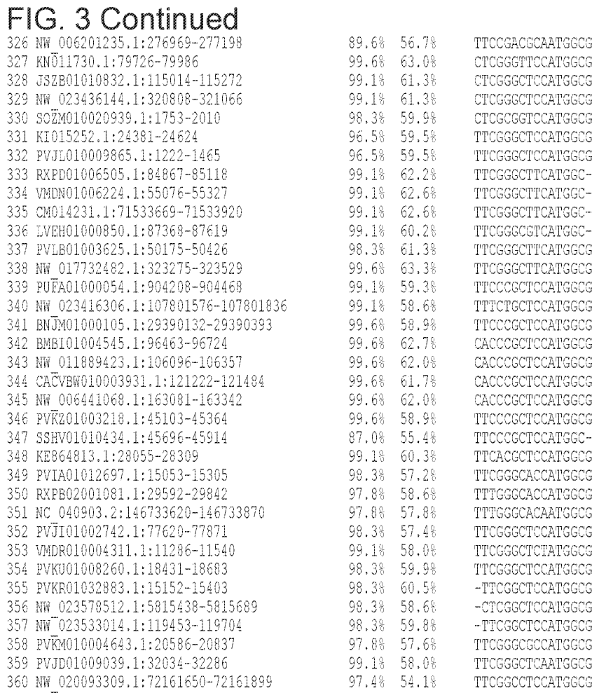

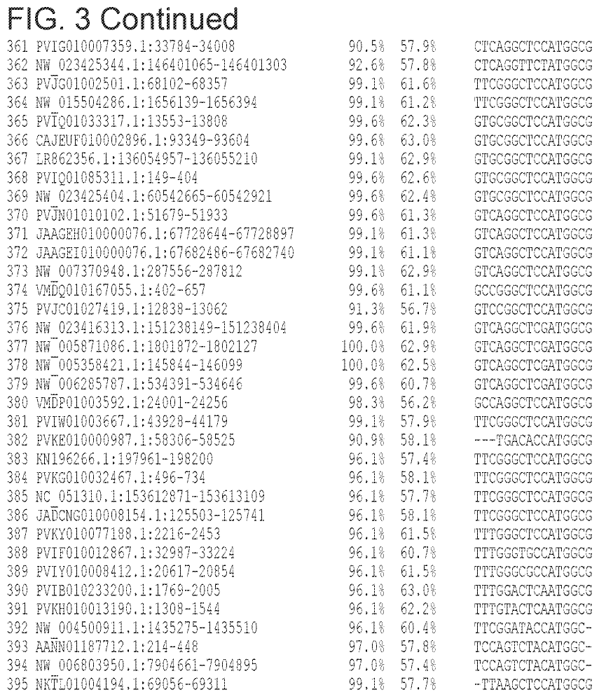

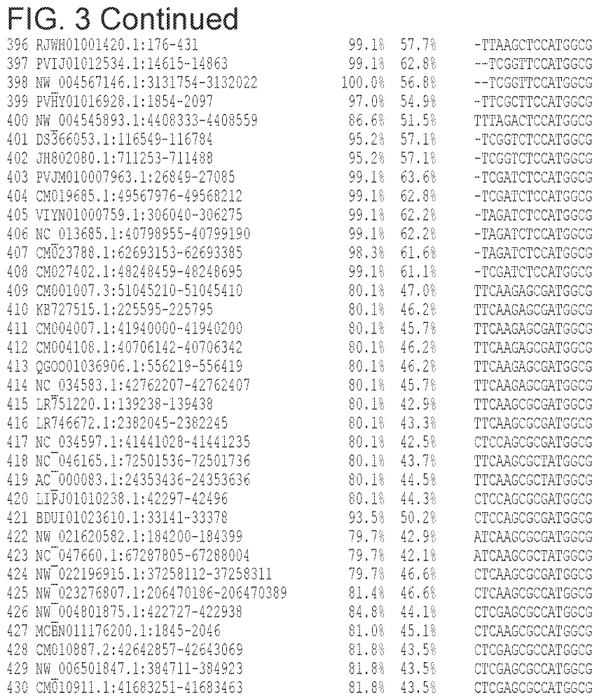

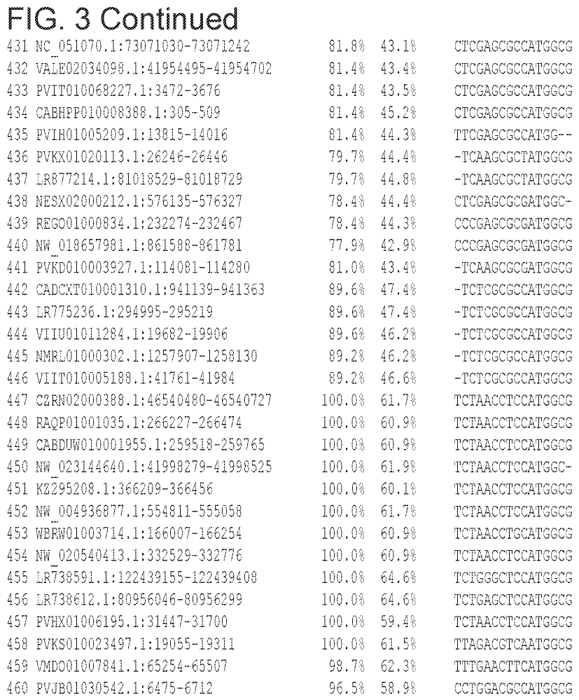

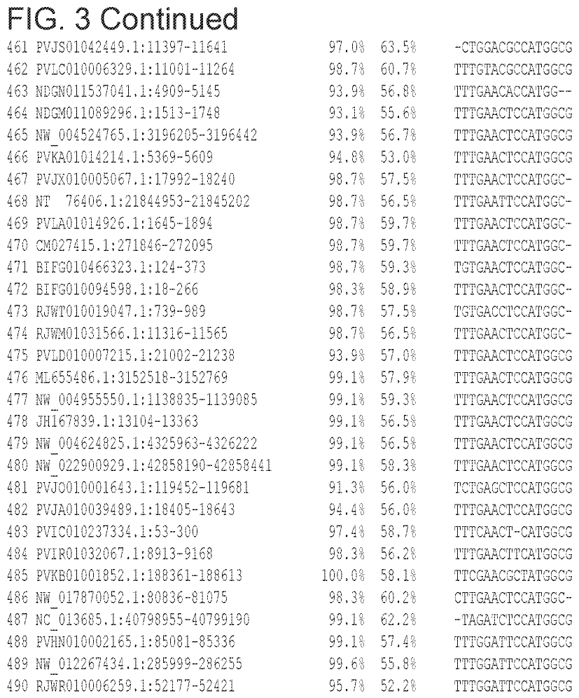

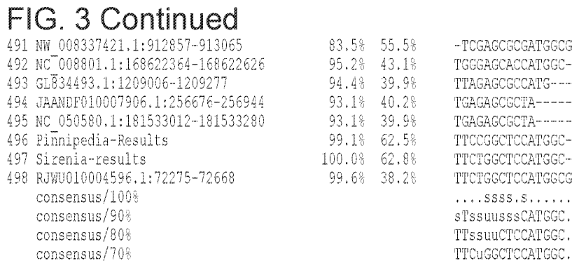

In certain embodiments, the promoter comprises a nucleotide sequence having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a functional fragment or variant (e.g., codon optimized) thereof. In some embodiments, the promoter comprises the nucleotide sequence of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a functional fragment or variant (e.g., codon optimized) thereof.

In certain embodiments, a functional fragment comprises a truncation of from about 10 to about 70 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 10 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 20 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 30 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 40 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 50 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 60 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 70 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)).

In certain embodiments, the promoter includes a group of promoters that shares a biological parameter (e.g., level of activity or ability to express in a certain cell or tissue, such as a lung cell). In certain embodiments, the compact promoter has higher activity than standard promoters (e.g., higher activity than a TK promoter).

In certain embodiments, the promoter includes a nucleotide sequence having at least 85%, 90%, 95%, 96%, 97%, 98%, 99%, or 100% identity to p104 (SEQ ID NO: 84 or a functional fragment or variant (e.g., codon optimized) thereof. In some embodiments, a functional fragment includes a truncation of from about 10 to about 70 bases (e.g., about 10 bases, about 20 bases, about 30 bases, about 40 bases, about 50 bases, about 60 bases, or about 70 bases) at the 5′ end, the 3′ end, or both the 5′ and 3′ ends of p104 (SEQ ID NO: 84).

In certain embodiments, the promoter is selected from a group of promoters consisting of p111 (SEQ ID NO: 29), p123 (SEQ ID NO: 34), or p128 (SEQ ID NO: 70), and the promoter has a nucleotide sequence having at least 85%, 90%, 95%, 96%, 97%, 98%, 99%, or 100% identity to p111 (SEQ ID NO: 29), p123 (SEQ ID NO: 34), or p128 (SEQ ID NO: 70), or a functional fragment or variant (e.g., codon optimized) thereof. In some embodiments, a functional fragment includes a truncation of from about 10 to about 70 bases (e.g., about 10 bases, about 20 bases, about 30 bases, about 40 bases, about 50 bases, about 60 bases, or about 70 bases) at the 5′ end, the 3′ end, or both the 5′ and 3′ ends of p111 (SEQ ID NO: 29), p123 (SEQ ID NO: 34), or p128 (SEQ ID NO: 70).

In certain embodiments, the promoter is selected from a group of promoters consisting of p064 (SEQ ID NO: 64), p082 (SEQ ID NO: 104), or p085 (SEQ ID NO: 54), and the promoter has a nucleotide sequence having at least 85%, 90%, 95%, 96%, 97%, 98%, 99%, or 100% identity to p064 (SEQ ID NO: 64), p082 (SEQ ID NO: 104), or p085 (SEQ ID NO: 54), or a functional fragment or variant (e.g., codon optimized) thereof. In some embodiments, a functional fragment includes a truncation of from about 10 to about 70 bases (e.g., about 10 bases, about 20 bases, about 30 bases, about 40 bases, about 50 bases, about 60 bases, or about 70 bases) at the 5′ end, the 3′ end, or both the 5′ and 3′ ends of p064 (SEQ ID NO: 64), p082 (SEQ ID NO: 104), or p085 (SEQ ID NO: 54).

In certain embodiments, the promoter is selected from a group of promoters consisting of p058 (SEQ ID NO: 96), p059 (SEQ ID NO: 92), p061 (SEQ ID NO: 65), p067 (SEQ ID NO: 52), p068 (SEQ ID NO: 103), p069 (SEQ ID NO: 62), p074 (SEQ ID NO: 99), p076 (SEQ ID NO: 51), p086 (SEQ ID NO: 83), p089 (SEQ ID NO: 43), p090 (SEQ ID NO: 40), p093 (SEQ ID NO: 46), p096 (SEQ ID NO: 87), p105 (SEQ ID NO: 44), p108 (SEQ ID NO: 32), p113 (SEQ ID NO: 78), p114 (SEQ ID NO: 39), p115 (SEQ ID NO: 77), p117 (SEQ ID NO: 35), p118 (SEQ ID NO: 37), p120 (SEQ ID NO: 73), p122 (SEQ ID NO: 30), p124 (SEQ ID NO: 31), p125 (SEQ ID NO: 76), p126 (SEQ ID NO: 76), or p129 (SEQ ID NO: 71), and the promoter has a nucleotide sequence having at least 85%, 90%, 95%, 96%, 97%, 98%, 99%, or 100% identity to p058 (SEQ ID NO: 96), p059 (SEQ ID NO: 92), p061 (SEQ ID NO: 65), p067 (SEQ ID NO: 52), p068 (SEQ ID NO: 103), p069 (SEQ ID NO: 62), p074 (SEQ ID NO: 99), p076 (SEQ ID NO: 51), p086 (SEQ ID NO: 83), p089 (SEQ ID NO: 43), p090 (SEQ ID NO: 40), p093 (SEQ ID NO: 46), p096 (SEQ ID NO: 87), p105 (SEQ ID NO: 44), p108 (SEQ ID NO: 32), p113 (SEQ ID NO: 78), p114 (SEQ ID NO: 39), p115 (SEQ ID NO: 77), p117 (SEQ ID NO: 35), p118 (SEQ ID NO: 37), p120 (SEQ ID NO: 73), p122 (SEQ ID NO: 30), p124 (SEQ ID NO: 31), p125 (SEQ ID NO: 76), p126 (SEQ ID NO: 72), or p129 (SEQ ID NO: 71), or a functional fragment or variant (e.g., codon optimized) thereof. In some embodiments, a functional fragment includes a truncation of from about 10 to about 70 bases (e.g., about 10 bases, about 20 bases, about 30 bases, about 40 bases, about 50 bases, about 60 bases, or about 70 bases) at the 5′ end, the 3′ end, or both the 5′ and 3′ ends of p058 (SEQ ID NO: 96), p059 (SEQ ID NO: 92), p061 (SEQ ID NO: 65), p067 (SEQ ID NO: 52), p068 (SEQ ID NO: 103), p069 (SEQ ID NO: 62), p074 (SEQ ID NO: 99), p076 (SEQ ID NO: 51), p086 (SEQ ID NO: 83), p089 (SEQ ID NO: 43), p090 (SEQ ID NO: 40), p093 (SEQ ID NO: 46), p096 (SEQ ID NO: 87), p105 (SEQ ID NO: 44), p108 (SEQ ID NO: 32), p113 (SEQ ID NO: 78), p114 (SEQ ID NO: 39), p115 (SEQ ID NO: 77), p117 (SEQ ID NO: 35), p118 (SEQ ID NO: 37), p120 (SEQ ID NO: 73), p122 (SEQ ID NO: 30), p124 (SEQ ID NO: 31), p125 (SEQ ID NO: 76), p126 (SEQ ID NO: 76), or p129 (SEQ ID NO: 71).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83. In certain embodiments, a functional fragment comprises at least a transcription factor binding sites selected from Staf, DSE, PSE, c-REL, GATA-1, GATA-2, and CREB. A functional fragment can comprise the B recognition sequence (BRE) or TATA box.

In certain embodiments, the promoter comprises a TATA mutation. In certain embodiments, the TATA mutation is a TATAA→TCGAA mutation.

In certain embodiments, the promoter is not one or more of SEQ ID NO: 70-SEQ ID NO: 106 and SEQ ID NO: 241-SEQ ID NO: 255. In certain embodiments, the promoter is not one or more of SEQ ID NO: 70-SEQ ID NO: 106. In certain embodiments, the promoter is not one of SEQ ID NO: 241-SEQ ID NO: 255.

In certain embodiments, a nucleic acid comprising a promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a promoter described herein further comprises a terminator sequence. In certain embodiments, the terminator sequence comprises one of the terminator sequences in TABLE 2.

TABLE 2

| | |
|---|---|
| a synthetic poly(A) | AATAAAATATCTTTATTTTCATTACATCTGTGTGTTGGTTTTTT |
| sequence (SPA) | GTGTG (SEQ ID NO: 258) |
| SPA and Pause | AATAAAATATCTTTATTTTCATTACATCTGTGTGTTGGTTTTTT |
| | GTGTGAATCGATAGTACTAACATACGCTCTCCATCAAAACAA |
| | AACGAAACAAAACAAACTAGCAAAATAGGCTGTCCCCAGTG |
| | CAAGTGCAGGTGCCAGAACATTTCTCT (SEQ ID NO: 259); |
| SV40 (240 bp) | ATCTAGATAACTGATCATAATCAGCCATACCACATTTGTAGA |
| | GGTTTTACTTGCTTTAAAAAACCTCCCACACCTCCCCCTGAAC |
| | CTGAAACATAAAATGAATGCAATTGTTGTTGTTAACTTGTTTA |
| | TTGCAGCTTATAATGGTTACAAATAAAGCAATAGCATCACAA |
| | ATTTCACAAATAAAGCATTTTTTTCACTGCATTCTAGTTGTGG |
| | TTTGTCCAAACTCATCAATGTATCTTA (SEQ ID NO: 260) |
| SV 40-mini | TTGTTTATTGCAGCTTATAATGGTTACAAATAAAGCAATAGC |
| (120 bp) | ATCACAAATTTCACAAATAAAGCATTTTTTTCACTGCATTCTA |
| | GTTGTGGTTTGTCCAAACTCATCAATGTATCTTAT (SEQ ID |
| | NO: 261) |
| bGH poly A | CGACTGTGCCTTCTAGTTGCCAGCCATCTGTTGTTTGCCCCTC |
| | CCCCGTGCCTTCCTTGACCCTGGAAGGTGCCACTCCCACTGTC |
| | CTTTCCTAATAAAATGAGGAAATTGCATCGCATTGTCTGAGT |
| | AGGTGTCATTCTATTCTGGGGGGTGGGGTGGGGCAGGACAGC |
| | AAGGGGGAGGATTGGGAAGACAATAGCAGGCATGCTGGGGA |
| | TGCGGTGGGCTCTATGG (SEQ ID NO: 262) |
| TKpoly A | GGGGGAGGCTAACTGAAACACGGAAGGAGACAATACCGGAA |
| | GGAACCCGCGCTATGACGGCAATAAAAAGACAGAATAAAAC |
| | GCACGGGTGTTGGGTCGTTTGTTCATAAACGCGGGGTTCGGT |
| | CCCAGGGCTGGCACTCTGTCGATACCCCACCGAGACCCCATT |
| | GGGGCCAATACGCCCGCGTTTCTTCCTTTTCCCCACCCCACCC |
| | CCCAAGTTCGGGTGAAGGCCCAGGGCTCGCAGCCAACGTCG |
| | GGGCGGCAGGCCCTGCCATAG (SEQ ID NO: 263) |
| SNRP1 | GGTATCAAATAAAATACGAAATGTGACAGATT (SEQ ID NO: |
| | 264) |
| SNRP1a | AAATAAAATACGAAATGTGACAGATT (SEQ ID NO: 265) |
| Histone H4B | GGTTGCTGATTTCTCCACAGCTTGCATTTCTGAACCAAAGGCC |
| | CTTTTCAGGGCCGCCCAACTAAACAAAAGAAGAGCTGTATCC |
| | ATTAAGTCAAGAAGC (SEQ ID NO: 266) |
| MALAT-1 | GATTCGTCAGTAGGGTTGTAAAGGTTTTTCTTTTCCTGAGAAA |
| | ACAACCTTTTGTTTTCTCAGGTTTTGCTTTTTGGCCTTTCCCTA |
| | GCTTTAAAAAAAAAAAAGCAAAAGACGCTGGTGGCTGGCAC |
| | TCCTGGTTTCCAGGACGGGGTTCAAGTCCCTGCGGTGTCTTTG |
| | CTT (SEQ ID NO: 267) |
| MALAT-comp14 | AAAGGTTTTTCTTTTCCTGAGAAATTTCTCAGGTTTTGCTTTTT |
| | AAAAAAAAAGCAAAAGACGCTGGTGGCTGGCACTCCTGGTT |
| | TCCAGGACGGGGTTCAAGTCCCTGCGGTGTCTTTGCTT (SEQ |
| | ID NO: 268) |

In certain embodiments, the compact promoter is coupled with a viral intron (e.g., an SV40i intron, a MVM intron, a Mv2 intron, an HNRNPH1 intron, chimeric introns or synthetic introns).

In certain embodiments, the compact promoter does not comprise a viral promoter and/or a synthetic promoter. In certain embodiments, the compact promoter does not comprise F5tg83.

In certain embodiments, the compact promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a compact promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### H1 Promoters

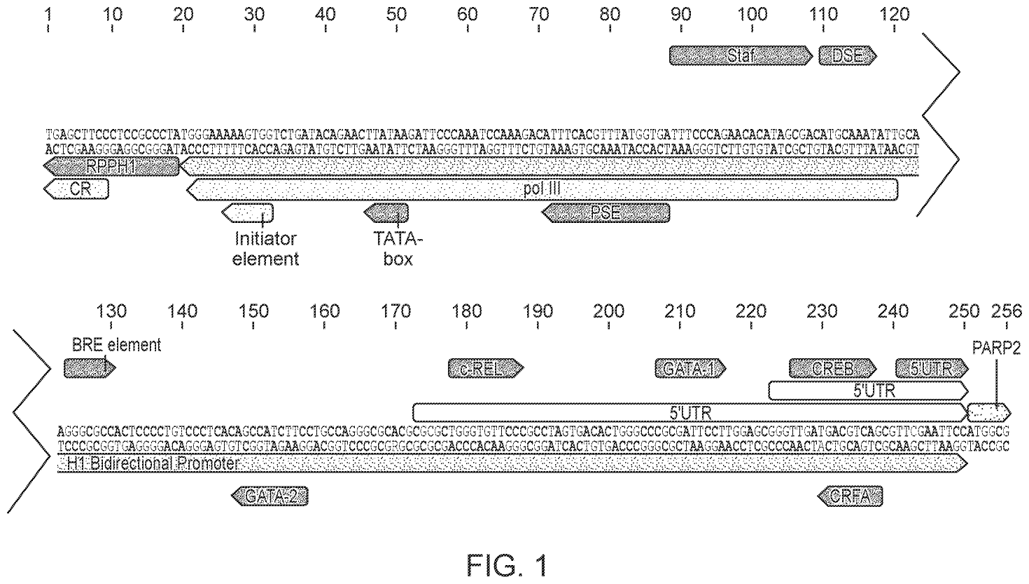

In certain embodiments, the promoter is comprises an H1 promoter. The H1 promoter is a bidirectional promoter having both pol II and pol III activity. The disclosure provides previously unidentified H1 promoters that Applicant identified by generating a Hidden Markov model (HMM) profile from a multispecies alignment of known H1 promoters (see, e.g., International Patent Publication No. WO2015/195621 and WO2018/009534). Regions flanking the H1 promoter region that were conserved throughout mammals were identified. As shown in FIG. 1, the region comprising the H1 promoter is located between the RPPH1 (H1 RNA) gene located on the minus strand to the left, and the beginning (i.e., the ATG(GCG)) of the protein coding gene, PARP2, located to the right. The RPPH1 gene comprises a highly conserved region in the H1 RNA gene (5′-GGAAGCTCA-3′) that is conserved throughout all mammals. Accordingly, in certain embodiments, the H1 promoter comprises or consists of a region between the ATG(GCG) of PARP2, and the highly conserved region in the H1 RNA gene (5′-GGAAGCTCA-3′). Also shown in FIG. 1 is the position of the pol III portion of the H1 promoter. Additional conserved regions present in the H1 promoter are shown, including, for example, conserved transcription factor binding sites, like a TATA box.

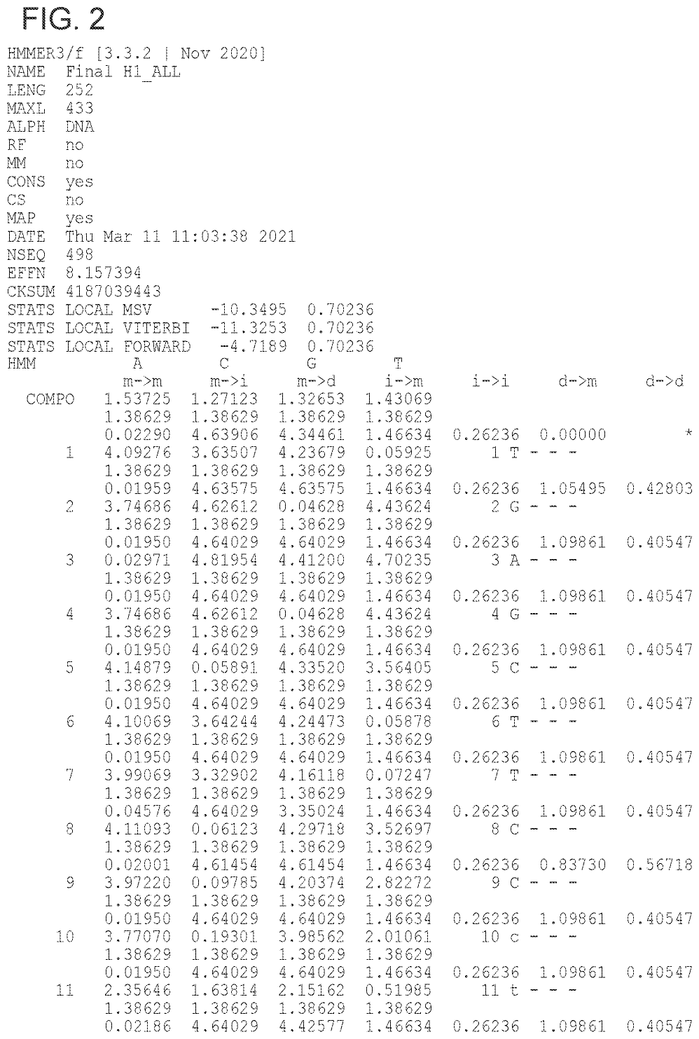

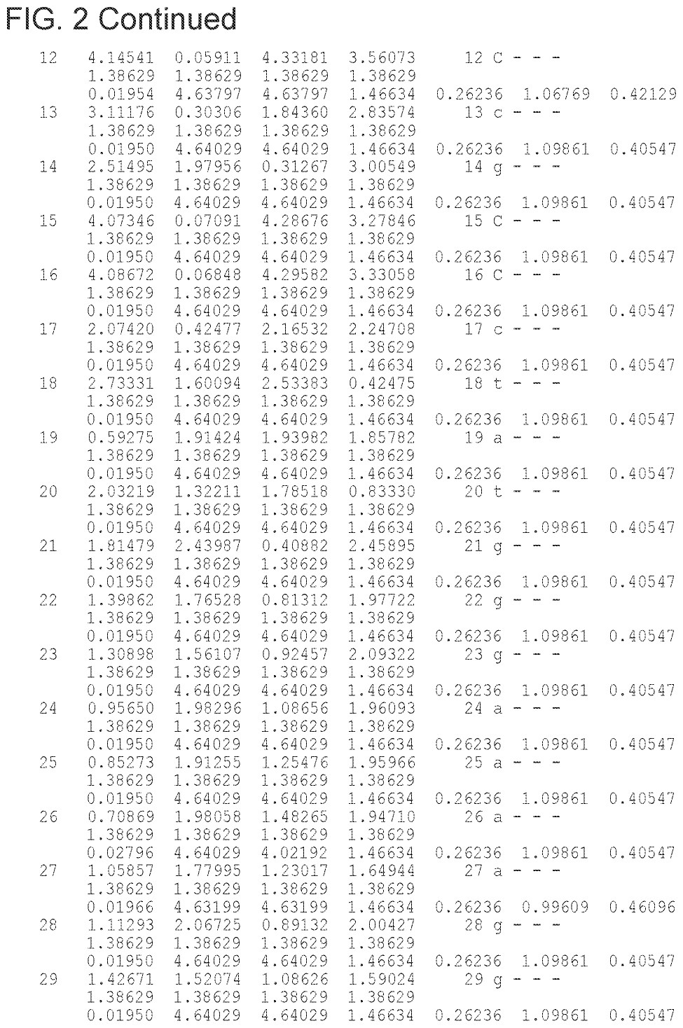

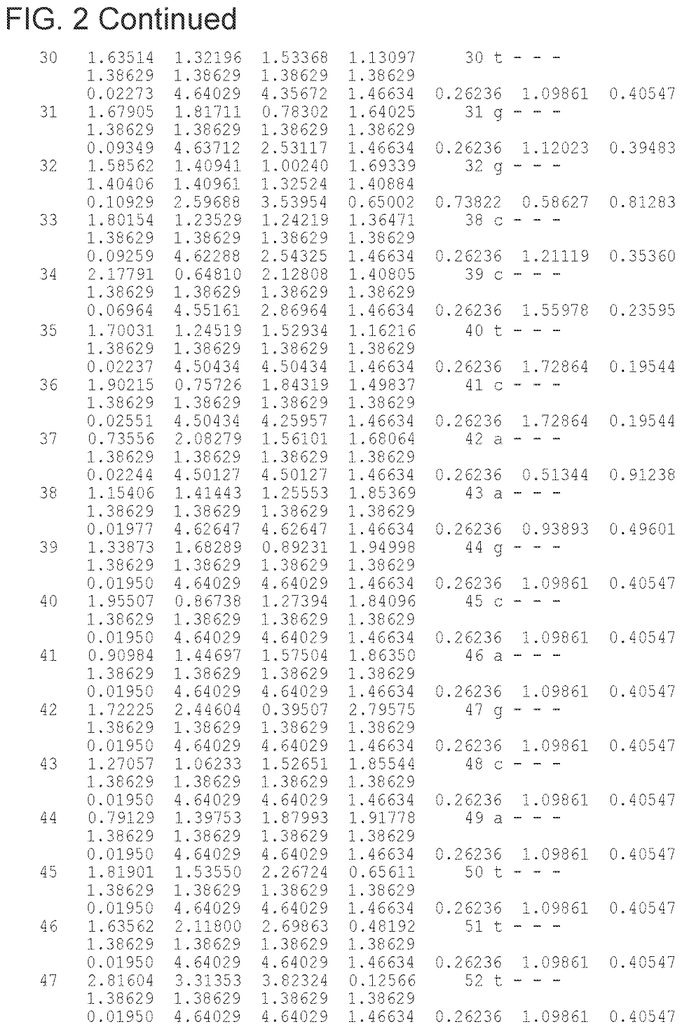

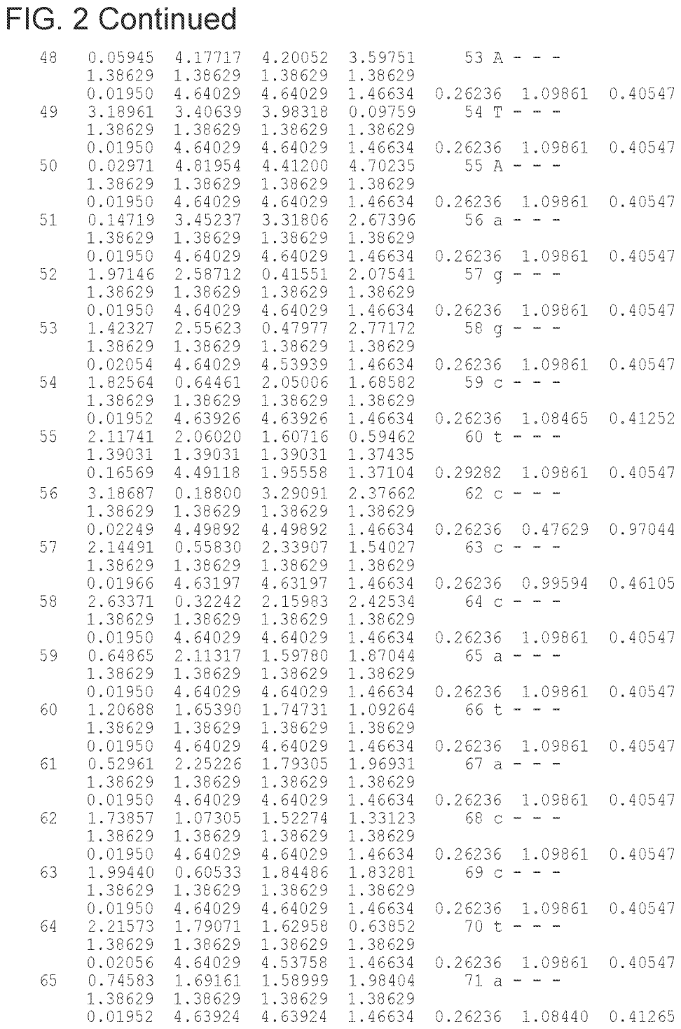

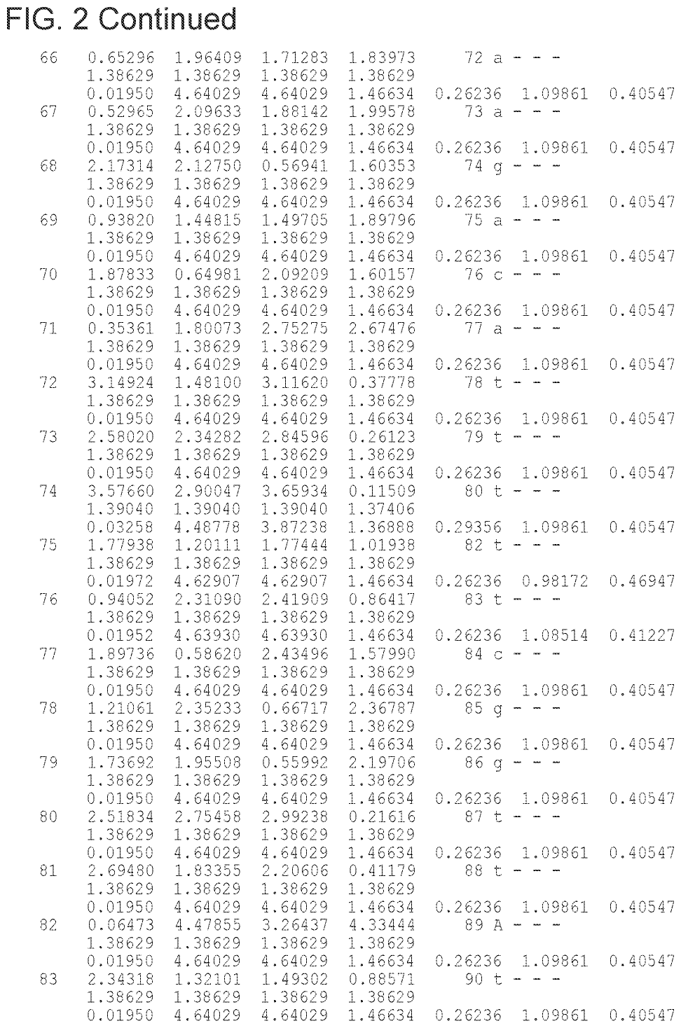

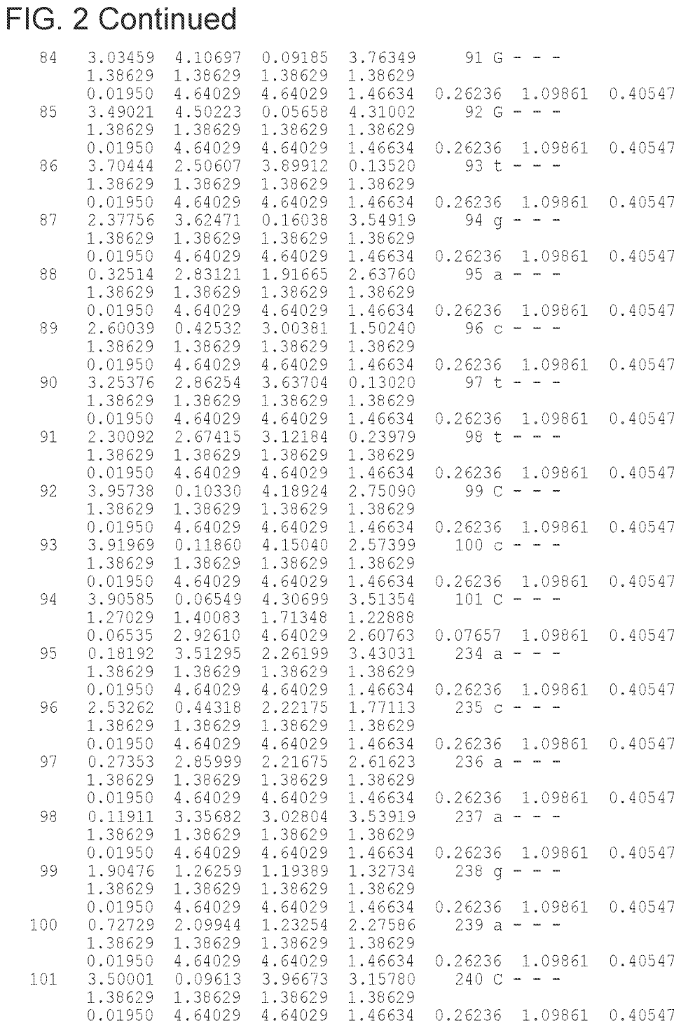

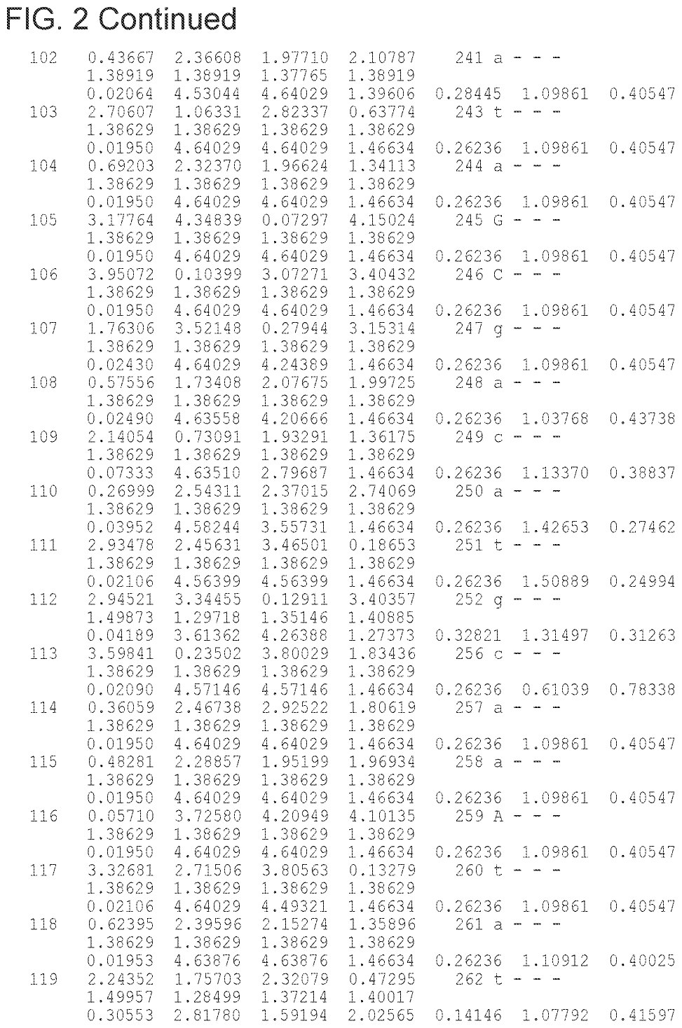

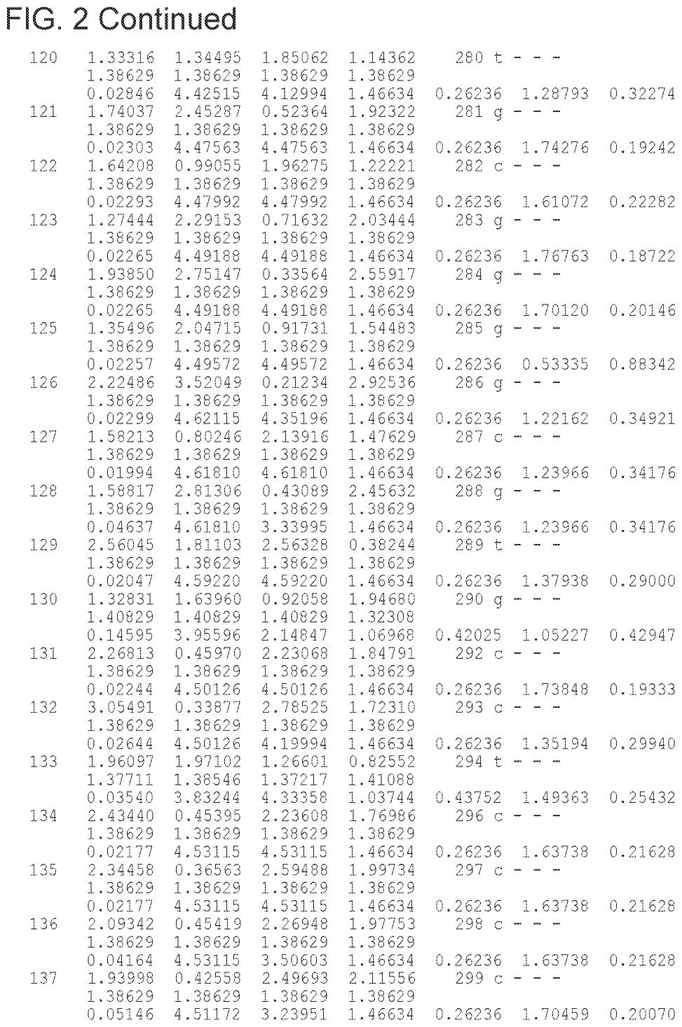

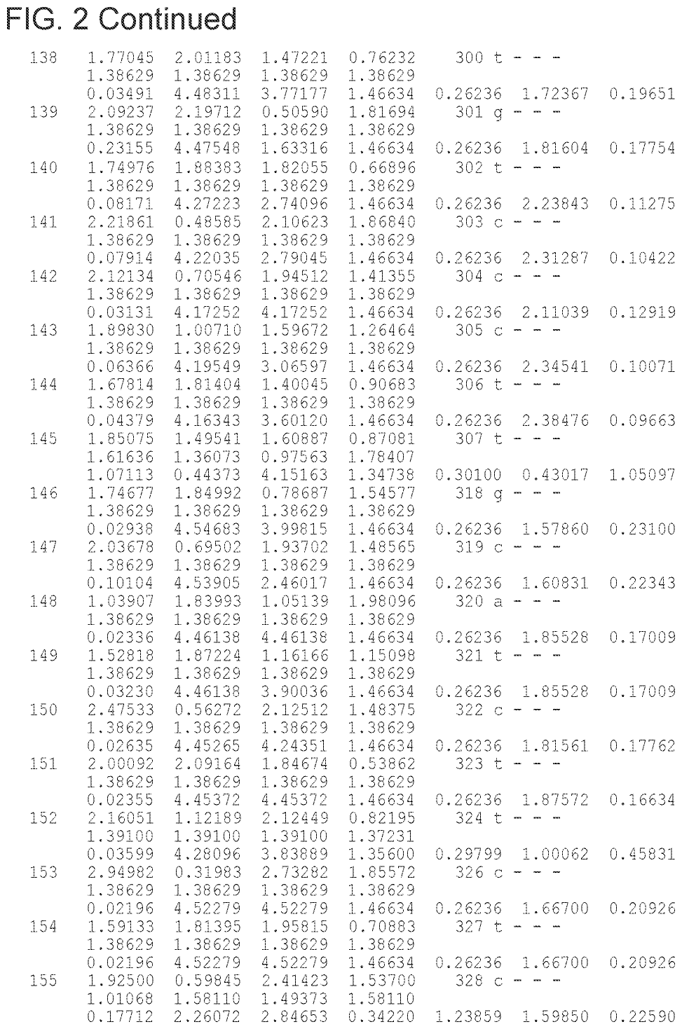

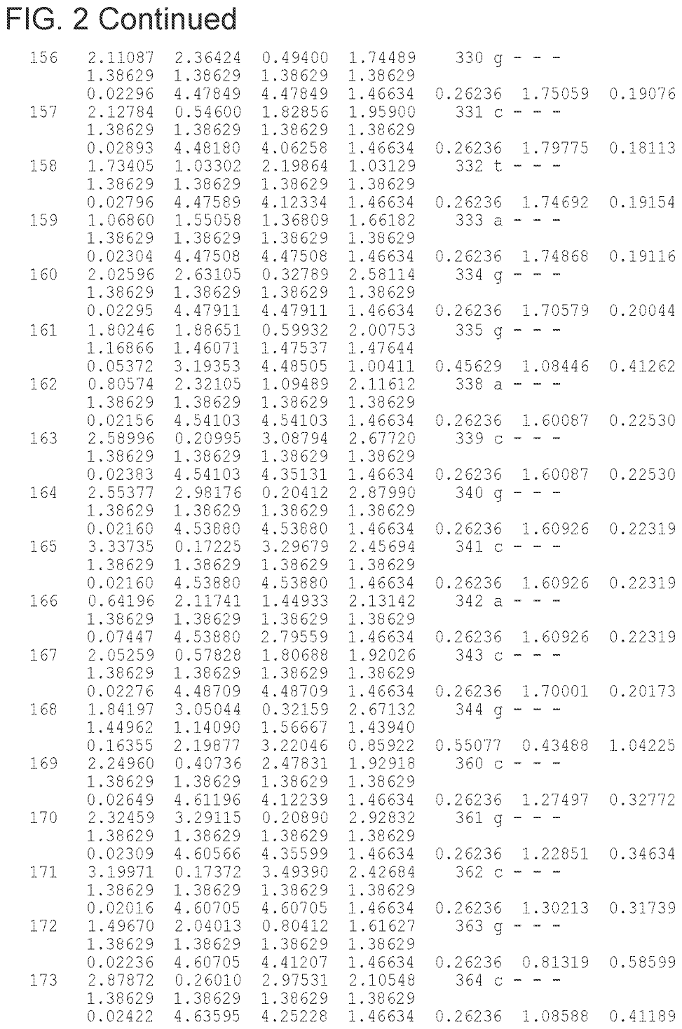

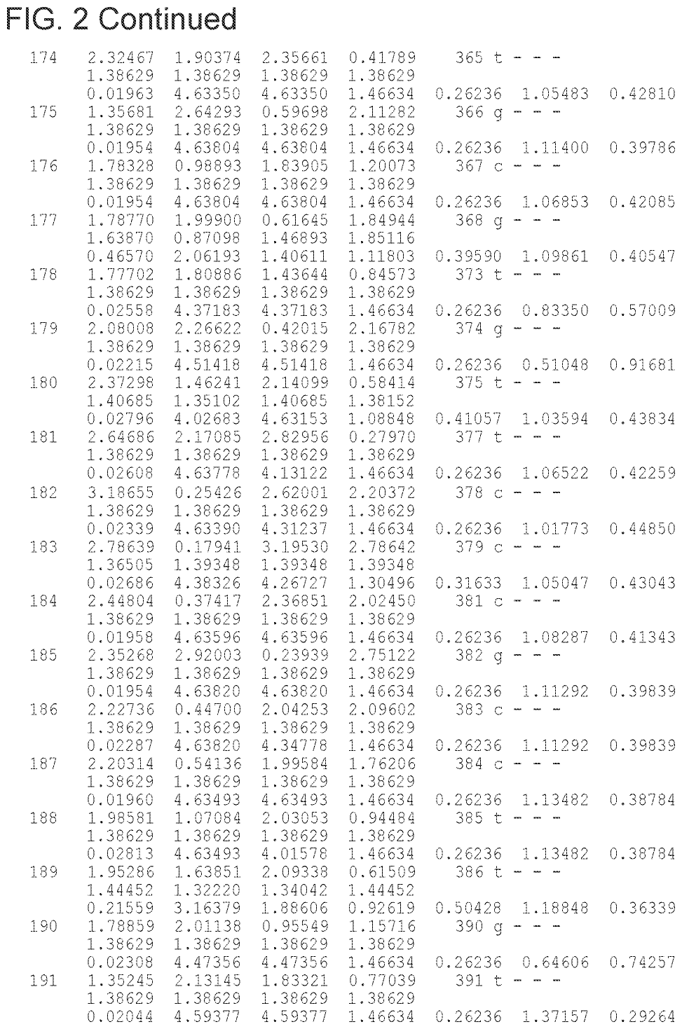

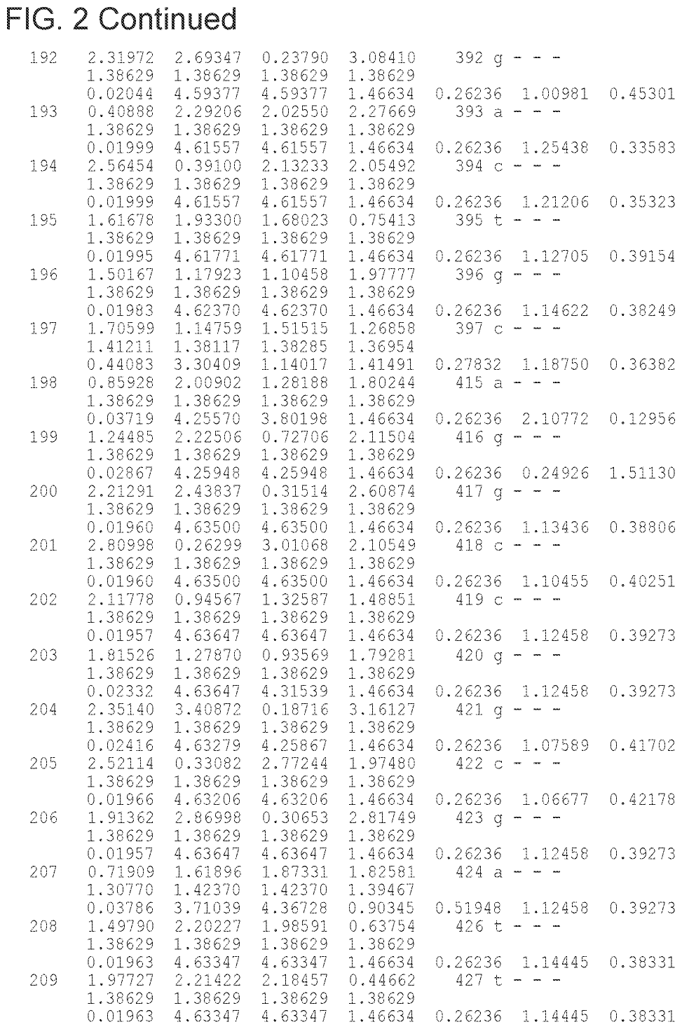

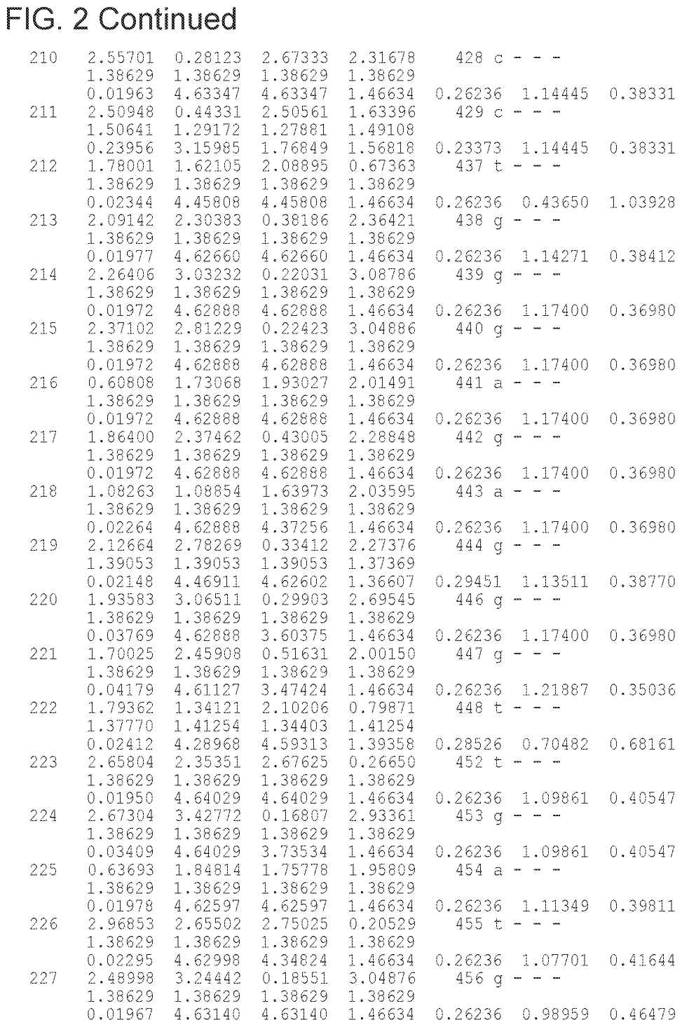

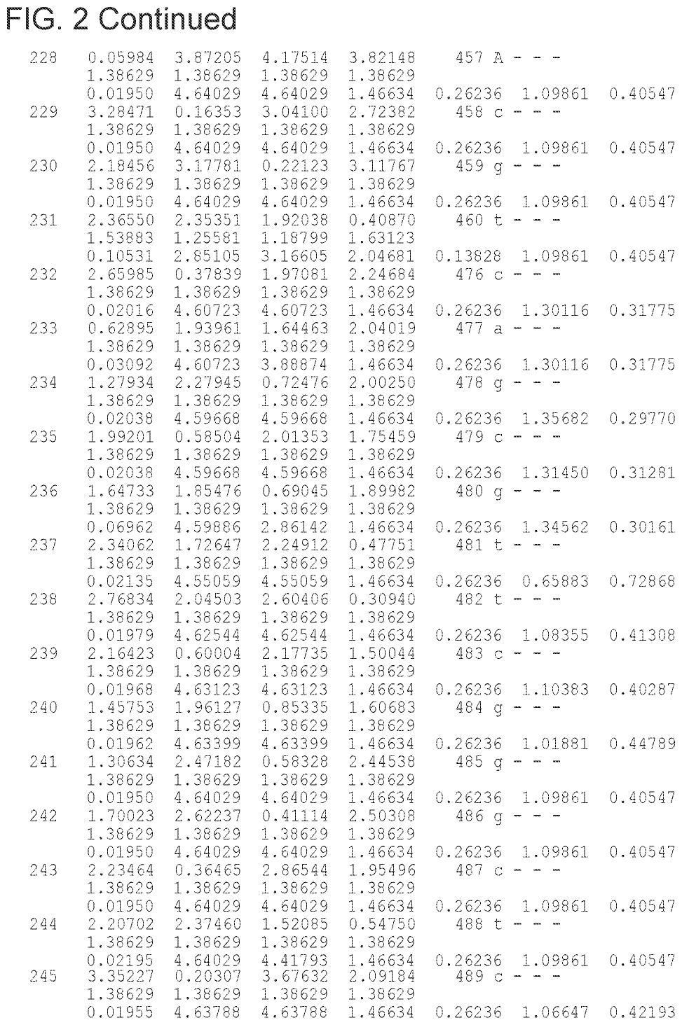

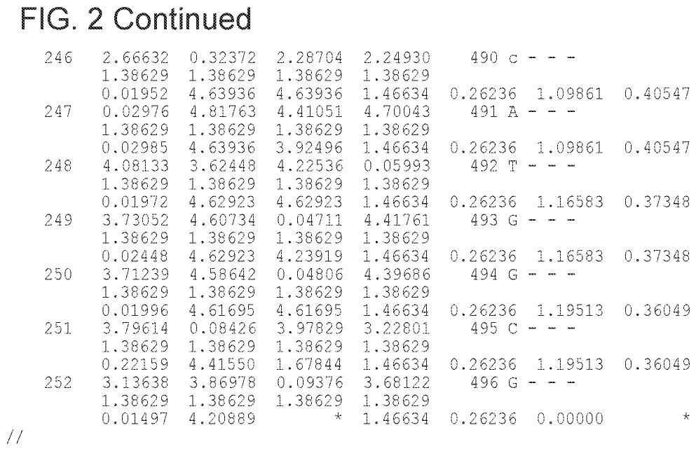

A Hidden Markov model (HMM) profile for identifying H1 promoters is provided in FIG. 2.

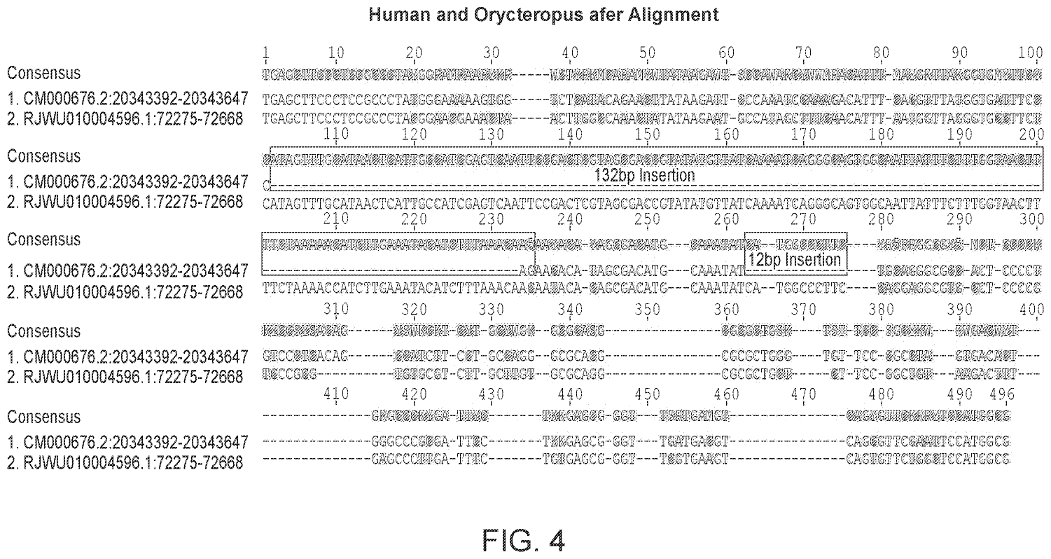

An alignment of naturally-occurring H1 promoters and consensus sequences is provided in FIG. 3 (wherein sequences numbered 1-498 in FIG. 3 correspond to SEQ ID NOs: 1304-1803 and consensus/100%, consensus/90%, consensus/80% and consensus/70% correspond to SEQ ID NOs: 1804-1807, respectively). Nucleotides 1-19 (as numbered in the alignment) form part of the H1 RNA gene and nucleotides 491 and above (as numbered in the alignment) form part of the PARP2 gene. Accordingly, nucleotides 20-490 correspond to the H1 promoter as used herein. Thus, in certain embodiments, the H1 promoter comprises nucleotides 20-490, as numbered in the alignment (or corresponding to the numbering in the alignment of FIG. 3 for a given H1 promoter sequence not present in the alignment of FIG. 3) of any one of SEQ ID NOs: 25-106 or a sequence provided in FIGS. 3-19. In addition, nucleotides 19-280, as numbered in the alignment (or corresponding to the numbering in the alignment of FIG. 3 for a given H1 promoter sequence not present in the alignment of FIG. 3)) of any one of SEQ ID NOs: 25-106 or a sequence provided in FIGS. 3-19 correspond with the pol III portion of the H1 promoter. An alignment of human and Orycteropus afer (Aardvark) H1 promoter sequences provided in FIG. 4 shows a 132 bp and a 12 bp insertion found in the Orycteropus afer H1 promoter sequence. Without wishing to be bound by theory, it is noted that the 144 bp insertion corresponds closely to the length of DNA required to wrap around a nucleosome (147 bp). Therefore, given the context of DNA found in eukaryotic cells, binding site distances are maintained and conserved.

In certain embodiments, the promoter is selected from a promoter in TABLE 3.

TABLE 3

| Promoter | Promoter | SEQ |
|---|---|---|
| Designation | Name | ID NO: |
| p095 | Marmoset H1 Bidirectional Promoter | 91 |
| p127 | Big brown bat H1 Bidirectional Promoter | 27 |
| p094 | Microbat H1 Bidirectional Promoter | 49 |
| p071 | Synthetic-2 H1 Bidirectional Promoter | 63 |
| p110 | Elephant H1 Bidirectional Promoter | 80 |
| p101 | Opossum H1 Bidirectional Promoter | 50 |
| p109 | David's myotis H1 Bidirectional Promoter | 38 |
| p116 | Bushbaby H1 Bidirectional Promoter | 74 |
| p066 | Star-nosed mole H1 Bidirectional Promoter | 61 |
| p060 | Tree Shrew H1 Bidirectional Promoter | 66 |
| p099 | Guinea pig H1 Bidirectional Promoter | 85 |
| p131 | Aardvark H1 Bidirectional Promoter | 25 |
| p100 | Goat H1 Bidirectional Promoter | 41 |
| p098 | Ferret H1 Bidirectional Promoter | 82 |
| p097 | Horse H1 Bidirectional Promoter | 86 |
| p092 | Killer whale H1 Bidirectional Promoter | 45 |
| p073 | Shrew H1 Bidirectional Promoter | 56 |
| p112 | Chinese tree shrew H1 Bidirectional Promoter | 36 |
| p081 | Sooty mangabey H1 Bidirectional Promoter | 59 |
| p078 | Shrew mouse H1 Bidirectional Promoter | 57 |
| p079 | Sheep H1 Bidirectional Promoter | 102 |
| p077 | Sifaka H1 Bidirectional Promoter | 58 |
| p065 | White-faced sapajou H1 Bidirectional Promoter | 69 |
| p130 | Angolan colobus H1 Bidirectional Promoter | 26 |
| p084 | Rat H1 Bidirectional Promoter | 100 |
| p106 | Cape golden mole H1 Bidirectional Promoter | 33 |
| p088 | Orangutan H1 Bidirectional Promoter | 95 |
| p091 | Mas night monkey H1 Bidirectional Promoter | 48 |
| p103 | Manatee H1 Bidirectional Promoter | 47 |
| p102 | Large flying fox H1 Bidirectional Promoter | 89 |
| p087 | Golden hamster H1 Bidirectional Promoter | 42 |
| p083 | Squirrel monkey H1 Bidirectional Promoter | 60 |
| p063 | Weddell seal H1 Bidirectional Promoter | 67 |
| p064 | Tenrec H1 Bidirectional Promoter | 64 |
| p072 | Pig H1 Bidirectional Promoter | 97 |
| p070 | Ryukyu mouse H1 Bidirectional Promoter | 55 |
| p119 | Cat H1 Bidirectional Promoter | 75 |
| p082 | Tarsier H1 Bidirectional Promoter | 104 |
| p059 | Mouse H1 Bidirectional Promoter | 92 |
| p058 | Panda H1 Bidirectional Promoter | 96 |
| p085 | Rhesus H1 Bidirectional Promoter | 54 |
| p062 | White rhinoceros H1 Bidirectional Promoter | 68 |
| p067 | Pig-tailed macaque H1 Bidirectional Promoter | 52 |
| p107 | Black flying-fox H1 Bidirectional Promoter | 28 |
| p061 | Tibetan antelope H1 Bidirectional Promoter | 65 |
| p086 | Gorilla H1 Bidirectional Promoter | 83 |
| p105 | Hedgehog H1 Bidirectional Promoter | 44 |
| p089 | Golden snub-nosed monkey H1 Bidirectional | 43 |
| | Promoter | |
| p096 | Human H1 Bidirectional Promoter | 87 |
| p090 | Gibbon H1 Bidirectional Promoter | 40 |
| p076 | Pacific walrus H1 Bidirectional Promoter | 51 |
| p113 | Crab-eating macaque H1 Bidirectional Promoter | 78 |
| p069 | Synthetic-1 H1 Bidirectional Promoter | 62 |
| p068 | Squirrel H1 Bidirectional Promoter | 103 |
| p093 | Lesser Egyptian jerboa H1 Bidirectional Promoter | 46 |
| p074 | Rabbit H1 Bidirectional Promoter | 99 |
| p125 | Chimp H1 Bidirectional Promoter | 76 |
| p124 | Brush-tailed rat H1 Bidirectional Promoter | 31 |
| p117 | Chinese hamster H1 Bidirectional Promoter | 35 |
| p114 | Drill H1 Bidirectional Promoter | 39 |
| p108 | Camel H1 Bidirectional Promoter | 32 |
| p118 | Consensus-1 H1 Bidirectional Promoter | 37 |
| p126 | Baboon H1 Bidirectional Promoter | 72 |
| p129 | Armadillo H1 Bidirectional Promoter | 71 |
| p111 | Black snub-nosed monkey H1 Bidirectional | 29 |
| | Promoter | |
| p122 | Bonobo H1 Bidirectional Promoter | 30 |
| p120 | Bottlenose dolphin H1 Bidirectional Promoter | 73 |
| p128 | Alpaca H1 Bidirectional Promoter | 70 |
| p104 | Green monkey H1 Bidirectional Promoter | 84 |
| p123 | Chinchilla H1 Bidirectional Promoter | 34 |
| p115 | Cow H1 Bidirectional Promoter | 77 |

In certain embodiments, the H1 promoter is a mammalian promoter, e.g., an artiodactyla H1 promoter, a camnivora H1 promoter, a cetacea H1 promoter, a chiroptera H1 promoter, an insectivora H1 promoter, a lagomorpha H1 promoter, a marsupial H1 promoter, a pangolin H1 promoter, a penissodactyla H1 promoter, a primate H1 promoter, a rodent H1 promoter, or a xenartha promoter. In certain embodiments, the H1 promoter is an ancestral promoter (e.g., selected from SEQ ID NOs: 936-1303). In some embodiments, the promoter comprises a nucleotide sequence having at least 85%, 90%, 95%, 96%, 97%, 98%, 99%, or 100% identity to the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3), or a functional fragment or variant (e.g., codon optimized) thereof. In some embodiments, the promoter comprises the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3), or a functional fragment or variant (e.g., codon optimized) thereof.

In certain embodiments, the promoter is not one or more of SEQ ID NO: 70-SEQ ID NO: 106.

In certain embodiments, a functional fragment comprises a truncation of from about 10 bases to about 40 bases at the 5′ end, the 3′ end, or both the 5′ and 3′ ends of any one of SEQ ID NOs: 25-106 or a sequence provided in FIGS. 3-19, or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 15 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 20 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 25 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 30 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 35 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of any one of SEQ ID NOs: 25-106 or a sequence provided in FIGS. 3-19). In certain embodiments, a functional fragment comprises a truncation of about 40 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the promoter comprises a TATA mutation. In certain embodiments, the TATA mutation is a TATAA→TCGAA mutation.

In certain embodiments, a nucleic acid comprising a promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a promoter described herein further comprises a terminator sequence. In certain embodiments, the terminator sequence comprises one of the terminator sequences in TABLE 4.

TABLE 4

| | |
|---|---|
| a synthetic poly(A) | AATAAAATATCTTTATTTTCATTACATCTGTGTGTTGGTTTTTT |
| sequence (SPA) | GTGTG (SEQ ID NO: 258) |
| SPA and Pause | AATAAAATATCTTTATTTTCATTACATCTGTGTGTTGGTTTTTT |
| | GTGTGAATCGATAGTACTAACATACGCTCTCCATCAAAACAA |
| | AACGAAACAAAACAAACTAGCAAAATAGGCTGTCCCCAGTG |
| | CAAGTGCAGGTGCCAGAACATTTCTCT (SEQ ID NO: 259); |
| SV40 (240 bp) | ATCTAGATAACTGATCATAATCAGCCATACCACATTTGTAGA |
| | GGTTTTACTTGCTTTAAAAAACCTCCCACACCTCCCCCTGAAC |
| | CTGAAACATAAAATGAATGCAATTGTTGTTGTTAACTTGTTTA |
| | TTGCAGCTTATAATGGTTACAAATAAAGCAATAGCATCACAA |
| | ATTTCACAAATAAAGCATTTTTTTCACTGCATTCTAGTTGTGG |
| | TTTGTCCAAACTCATCAATGTATCTTA (SEQ ID NO: 260) |
| SV 40-mini | TTGTTTATTGCAGCTTATAATGGTTACAAATAAAGCAATAGC |
| (120 bp) | ATCACAAATTTCACAAATAAAGCATTTTTTTCACTGCATTCTA |
| | GTTGTGGTTTGTCCAAACTCATCAATGTATCTTAT (SEQ ID |
| | NO: 261) |
| bGH poly A | CGACTGTGCCTTCTAGTTGCCAGCCATCTGTTGTTTGCCCCTC |
| | CCCCGTGCCTTCCTTGACCCTGGAAGGTGCCACTCCCACTGTC |
| | CTTTCCTAATAAAATGAGGAAATTGCATCGCATTGTCTGAGT |
| | AGGTGTCATTCTATTCTGGGGGGTGGGGTGGGGCAGGACAGC |
| | AAGGGGGAGGATTGGGAAGACAATAGCAGGCATGCTGGGGA |
| | TGCGGTGGGCTCTATGG (SEQ ID NO: 262) |
| TKpoly A | GGGGGAGGCTAACTGAAACACGGAAGGAGACAATACCGGAA |
| | GGAACCCGCGCTATGACGGCAATAAAAAGACAGAATAAAAC |
| | GCACGGGTGTTGGGTCGTTTGTTCATAAACGCGGGGTTCGGT |
| | CCCAGGGCTGGCACTCTGTCGATACCCCACCGAGACCCCATT |
| | GGGGCCAATACGCCCGCGTTTCTTCCTTTTCCCCACCCCACCC |
| | CCCAAGTTCGGGTGAAGGCCCAGGGCTCGCAGCCAACGTCG |
| | GGGCGGCAGGCCCTGCCATAG (SEQ ID NO: 263) |
| SNRP1 | GGTATCAAATAAAATACGAAATGTGACAGATT (SEQ ID NO: |
| | 264) |
| SNRP1a | AAATAAAATACGAAATGTGACAGATT (SEQ ID NO: 265) |
| Histone H4B | GGTTGCTGATTTCTCCACAGCTTGCATTTCTGAACCAAAGGCC |
| | CTTTTCAGGGCCGCCCAACTAAACAAAAGAAGAGCTGTATCC |
| | ATTAAGTCAAGAAGC (SEQ ID NO: 266) |
| MALAT-1 | GATTCGTCAGTAGGGTTGTAAAGGTTTTTCTTTTCCTGAGAAA |
| | ACAACCTTTTGTTTTCTCAGGTTTTGCTTTTTGGCCTTTCCCTA |
| | GCTTTAAAAAAAAAAAAGCAAAAGACGCTGGTGGCTGGCAC |
| | TCCTGGTTTCCAGGACGGGGTTCAAGTCCCTGCGGTGTCTTTG |
| | CTT (SEQ ID NO: 267) |
| MALAT-comp14 | AAAGGTTTTTCTTTTCCTGAGAAATTTCTCAGGTTTTGCTTTTT |
| | AAAAAAAAAGCAAAAGACGCTGGTGGCTGGCACTCCTGGTT |
| | TCCAGGACGGGGTTCAAGTCCCTGCGGTGTCTTTGCTT (SEQ |
| | ID NO: 268) |

In certain embodiments, the compact promoter is coupled with a viral intron (e.g., an SV40i intron, a MVM intron, a Mv2 intron, an HNRNPH1 intron, chimeric introns or synthetic introns.).

In certain embodiments, the compact promoter does not comprise a viral promoter and/or a synthetic promoter. In certain embodiments, the compact promoter does not comprise F5tg83.

In certain embodiments, the compact promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a compact promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### Artiodactyla H1 Promoters

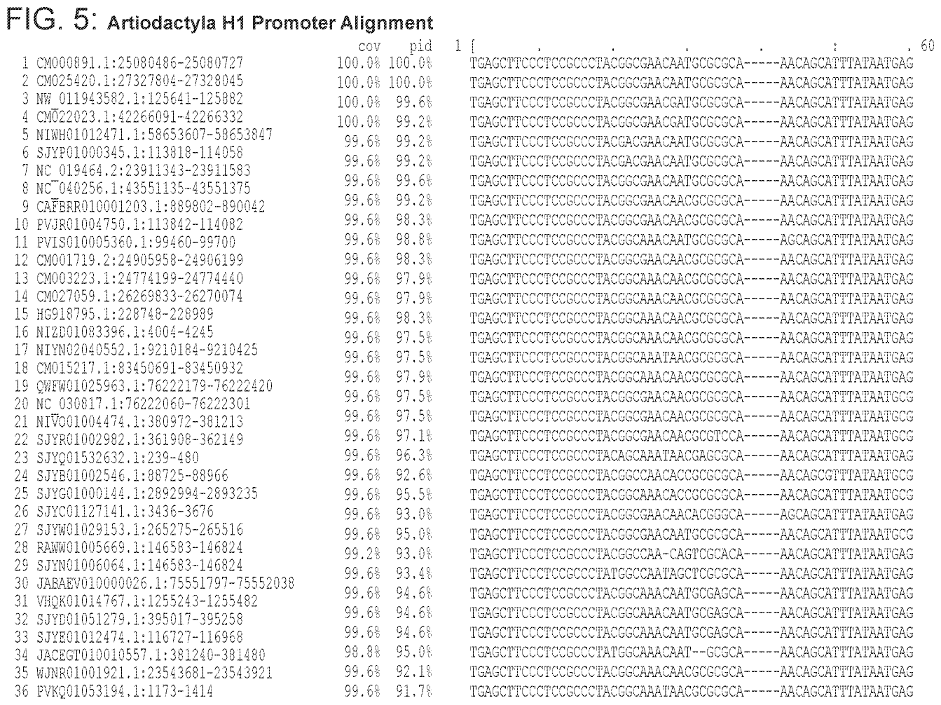

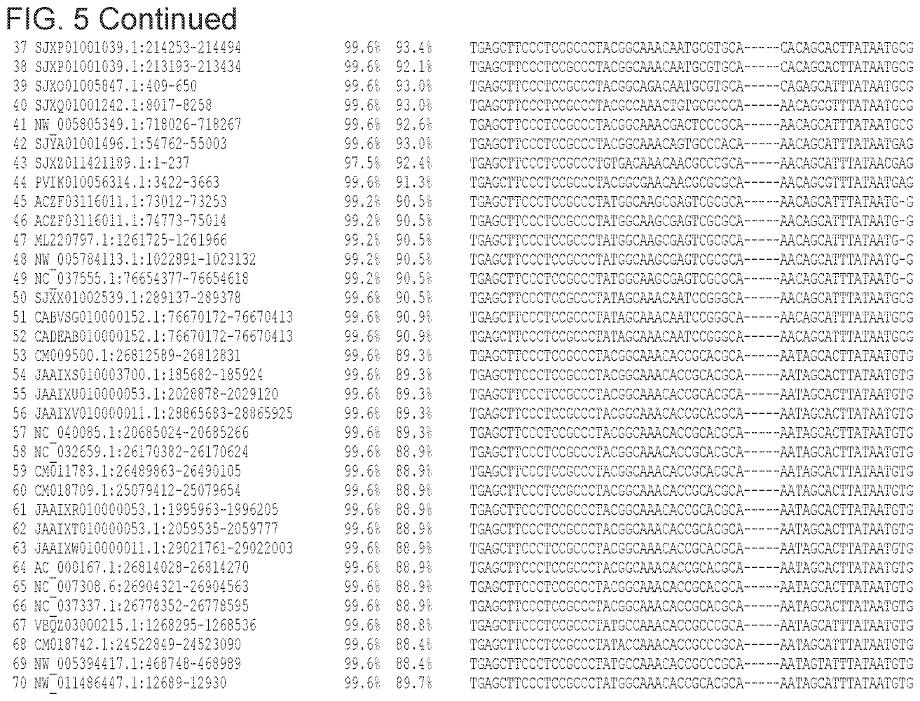

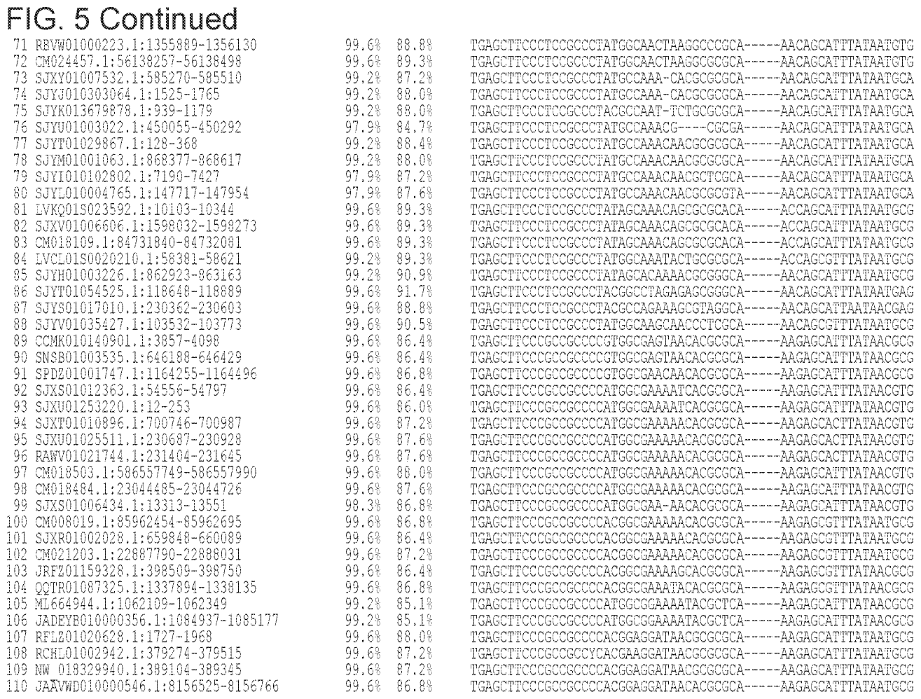

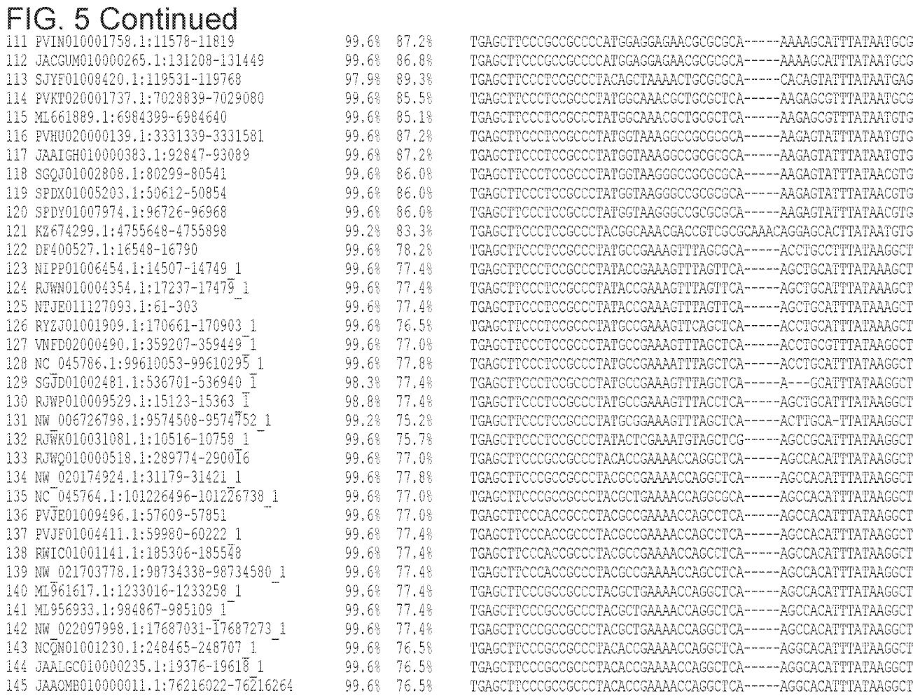

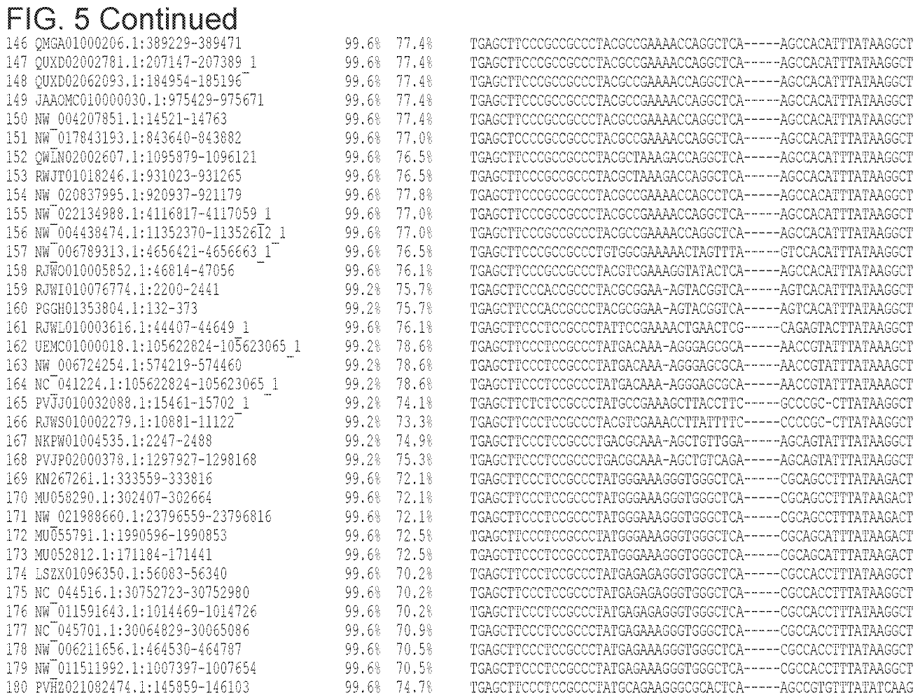

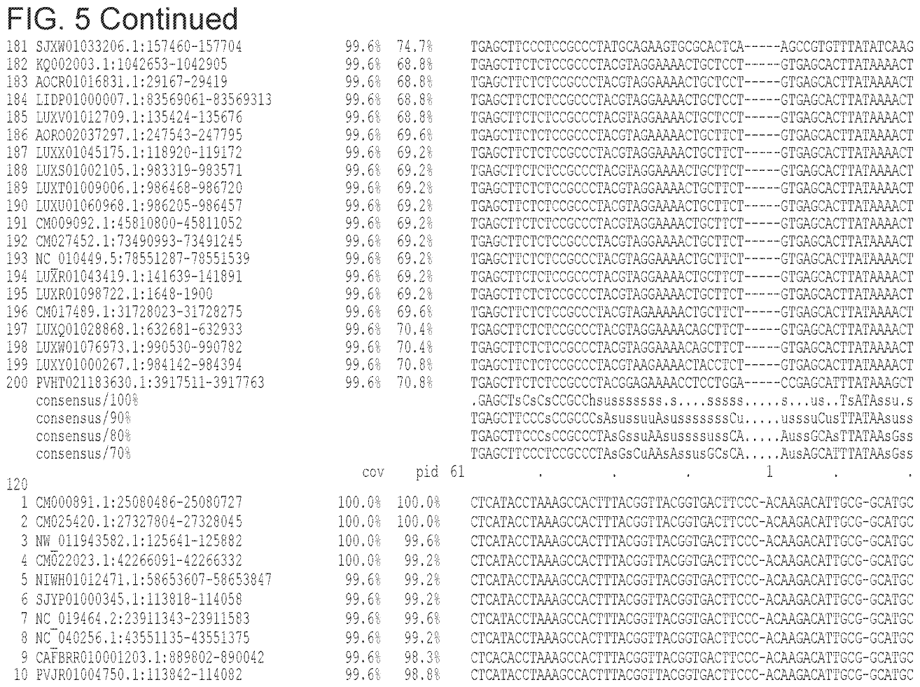

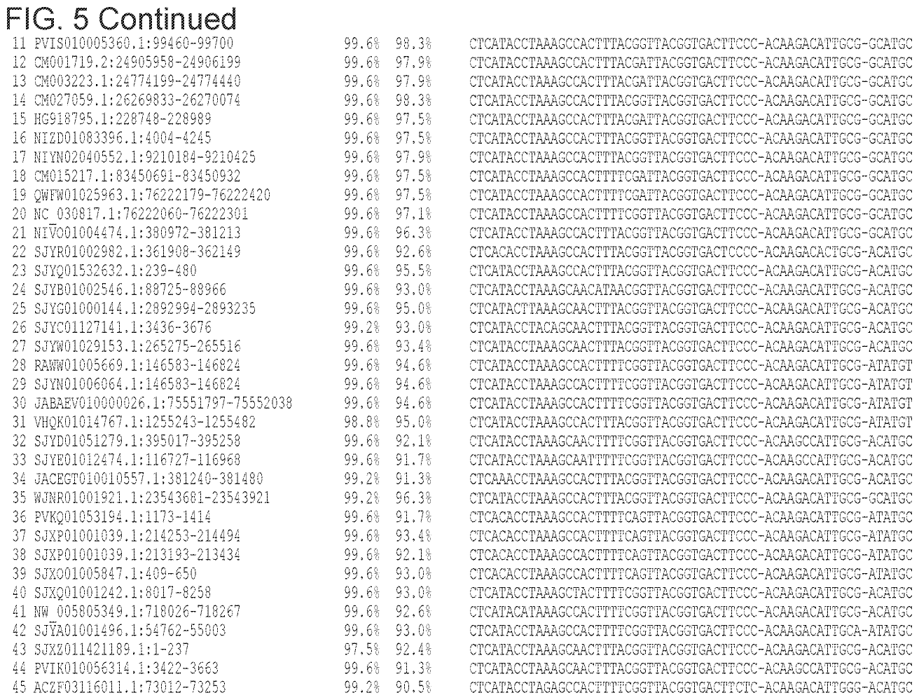

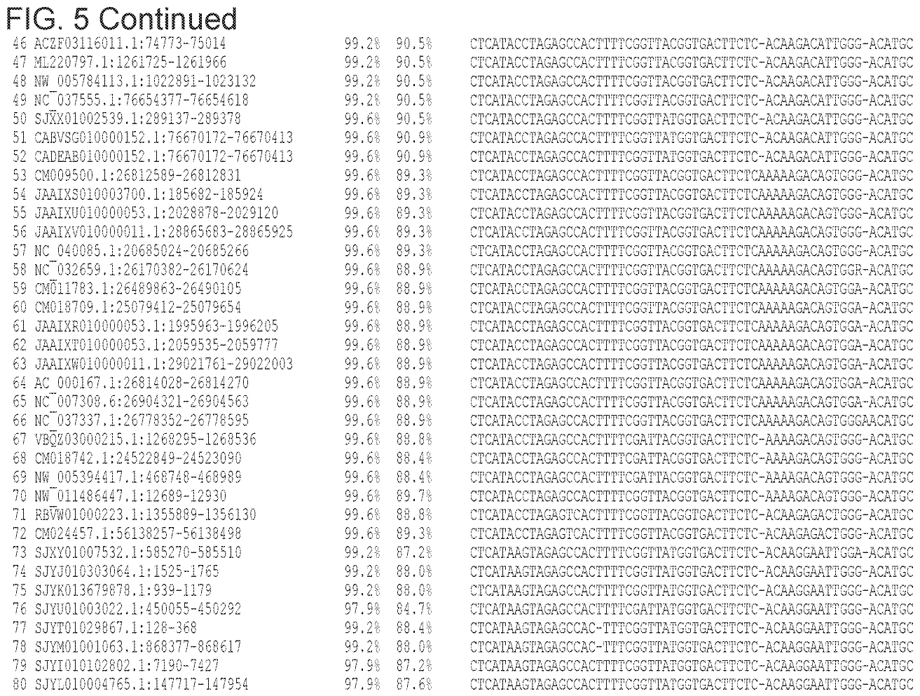

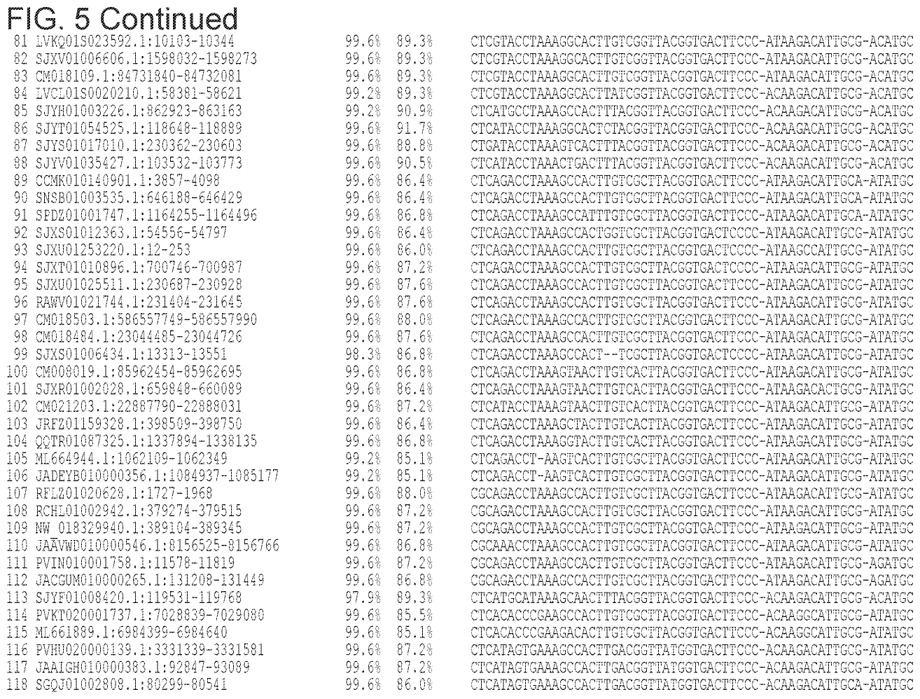

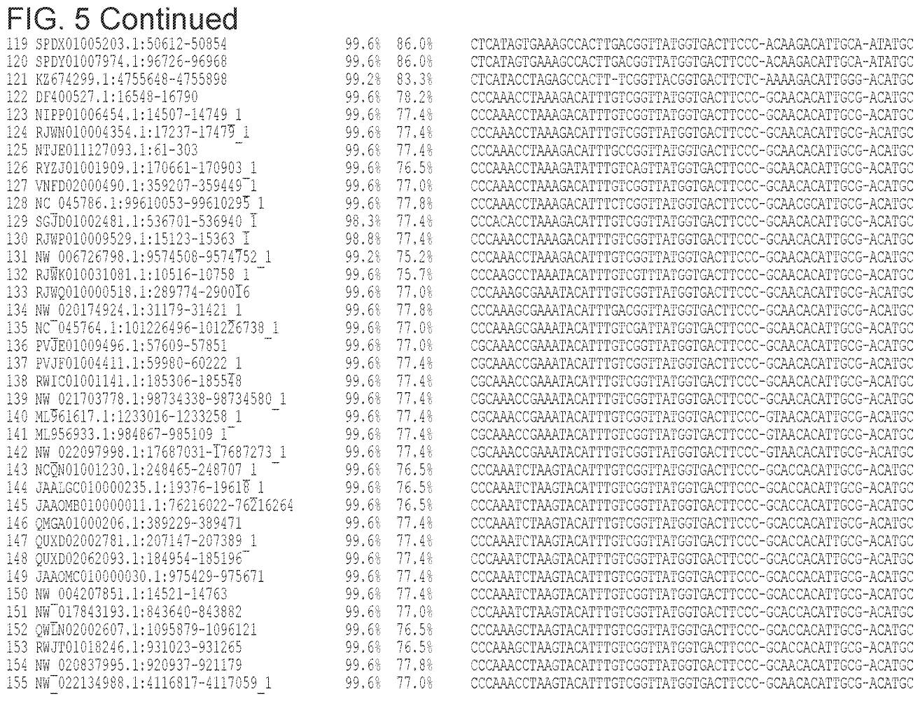

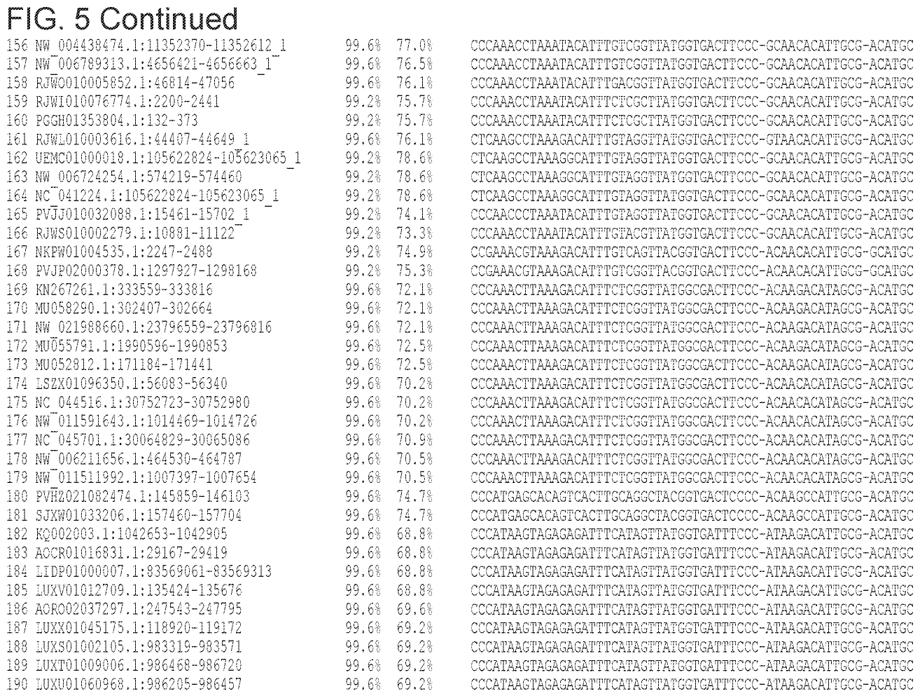

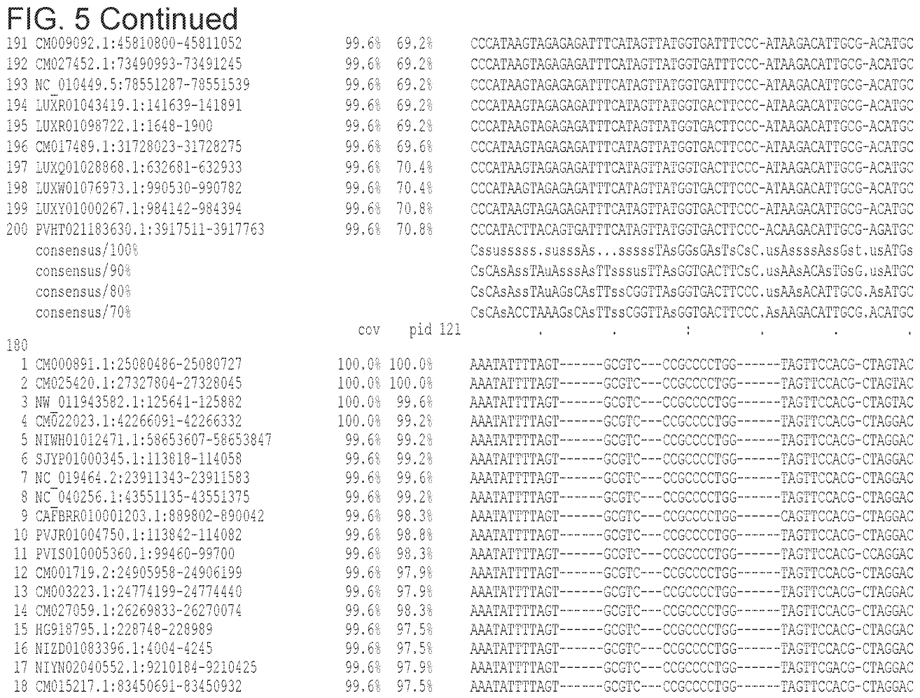

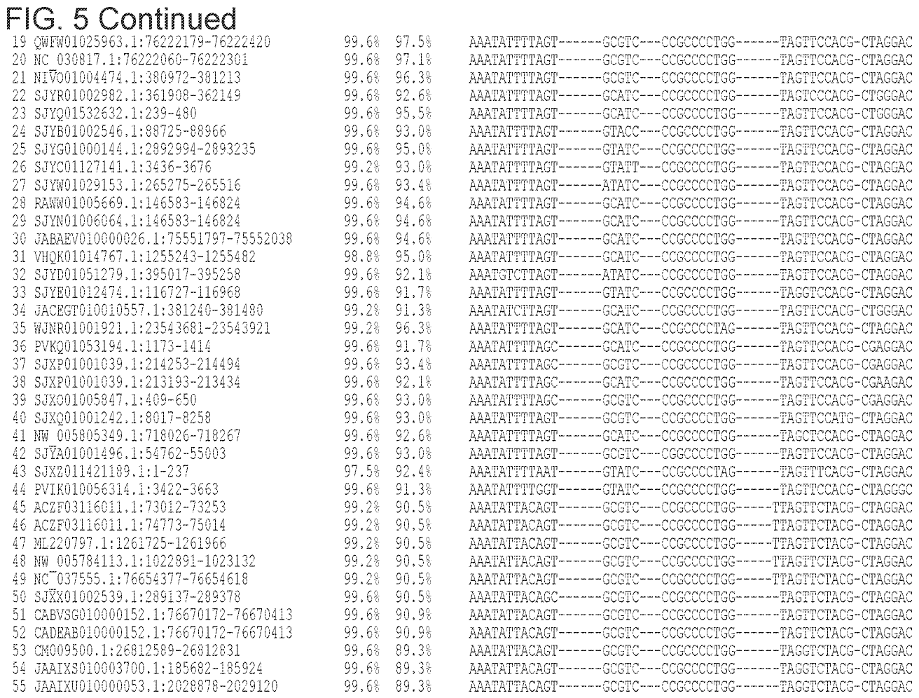

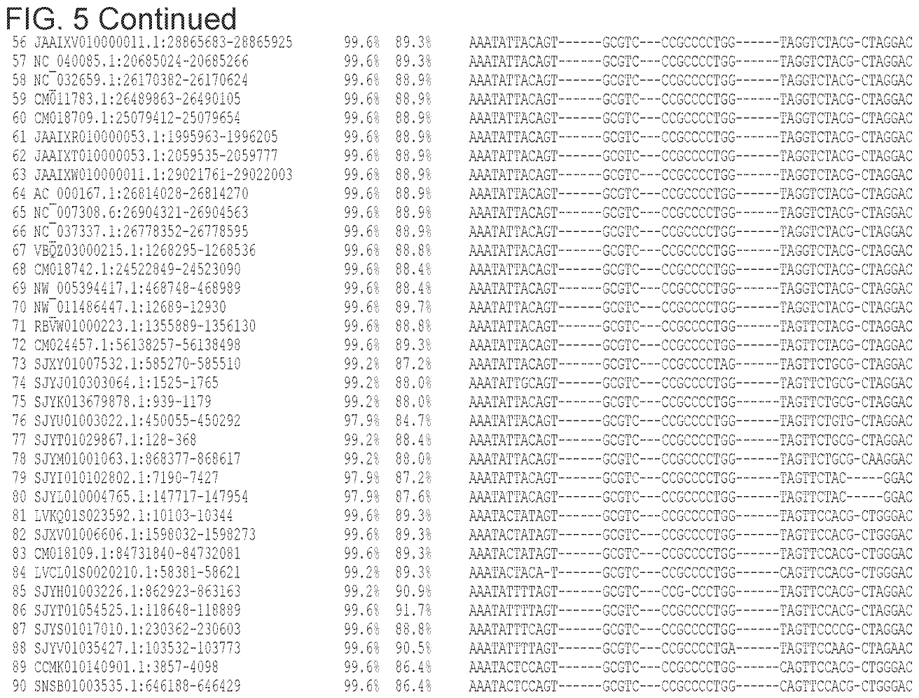

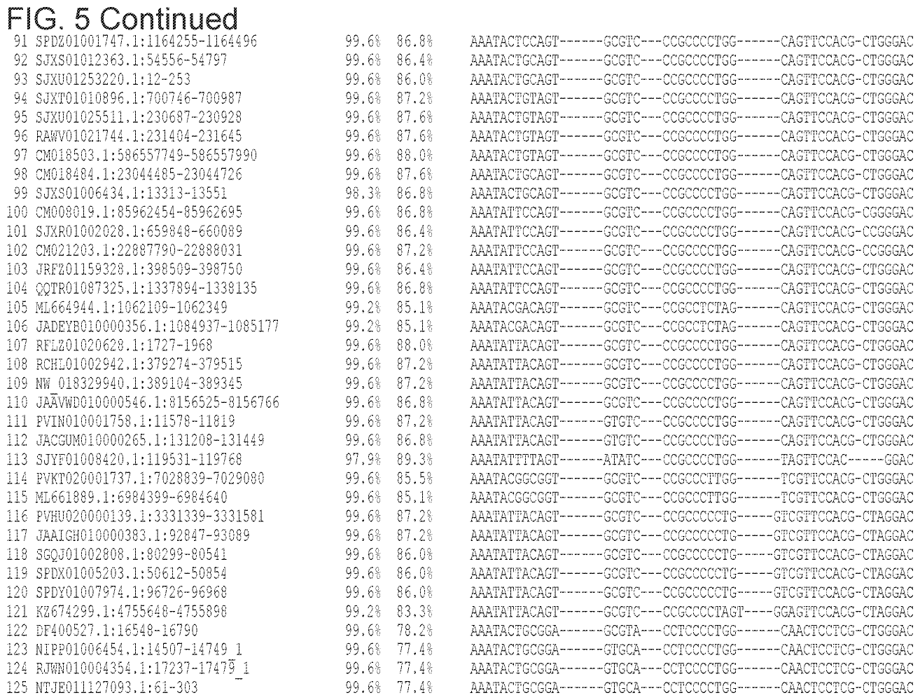

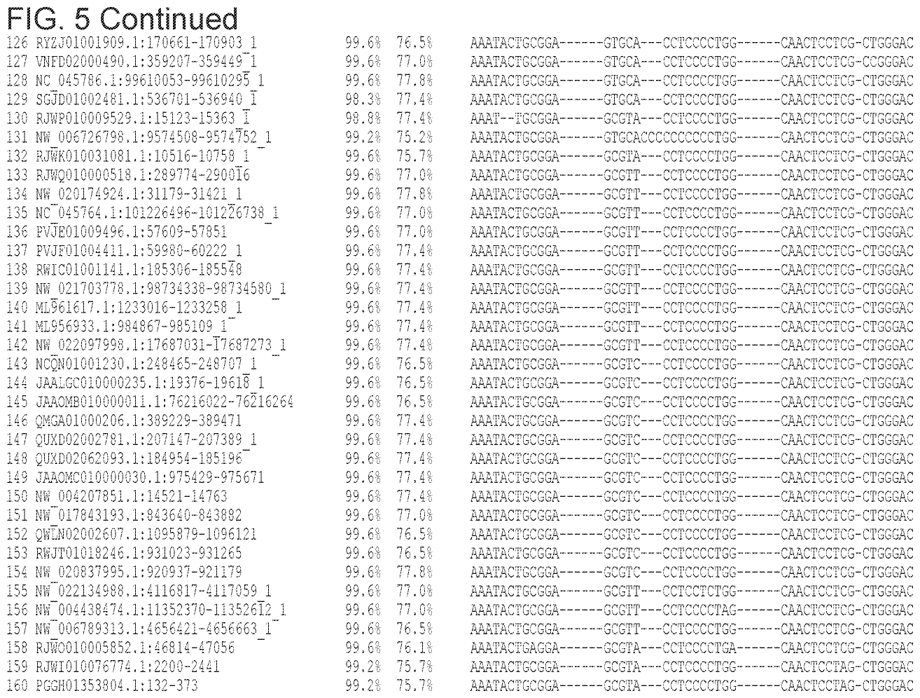

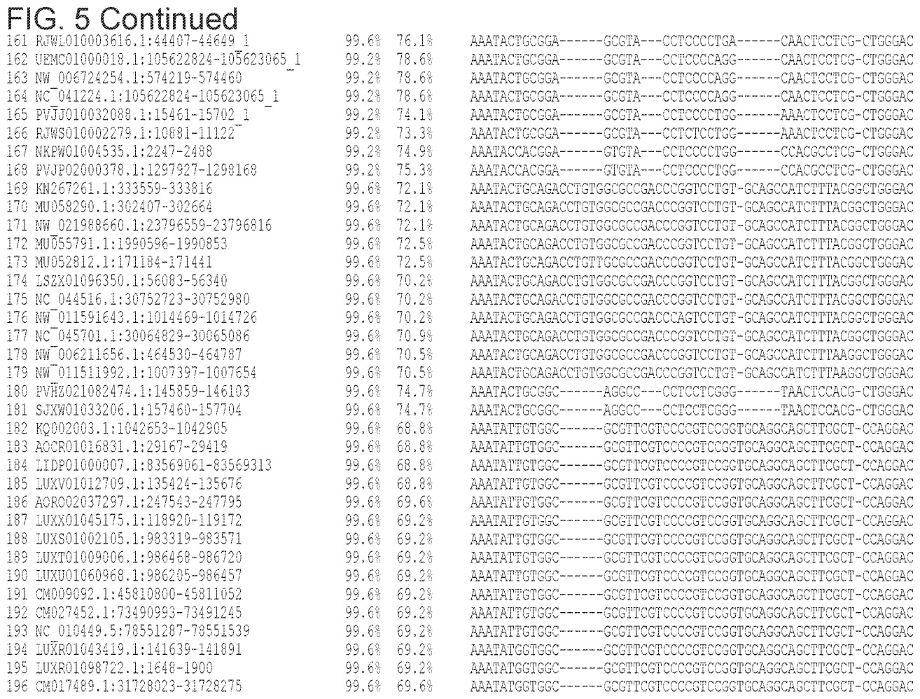

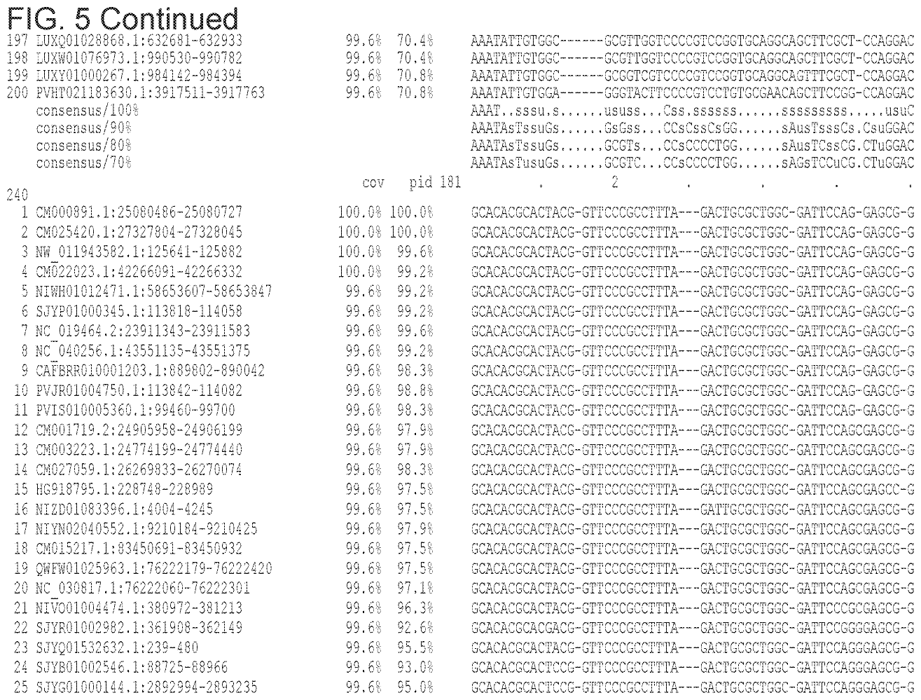

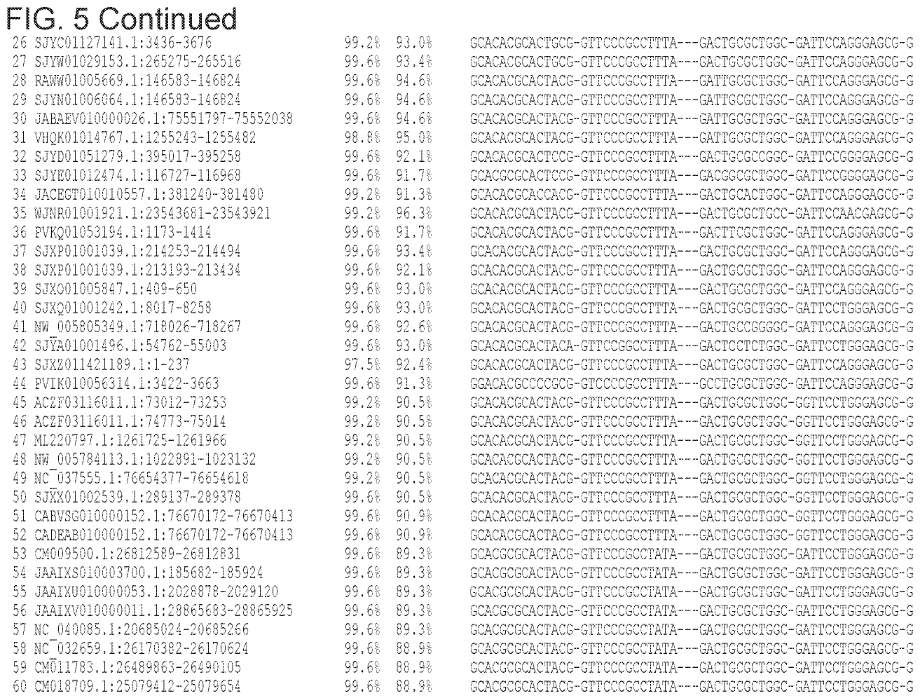

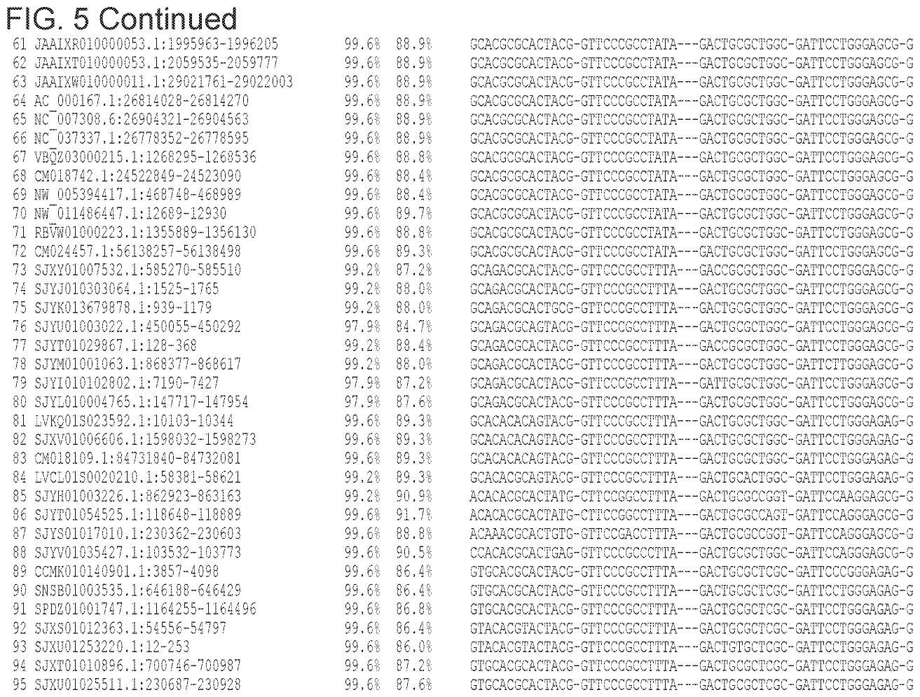

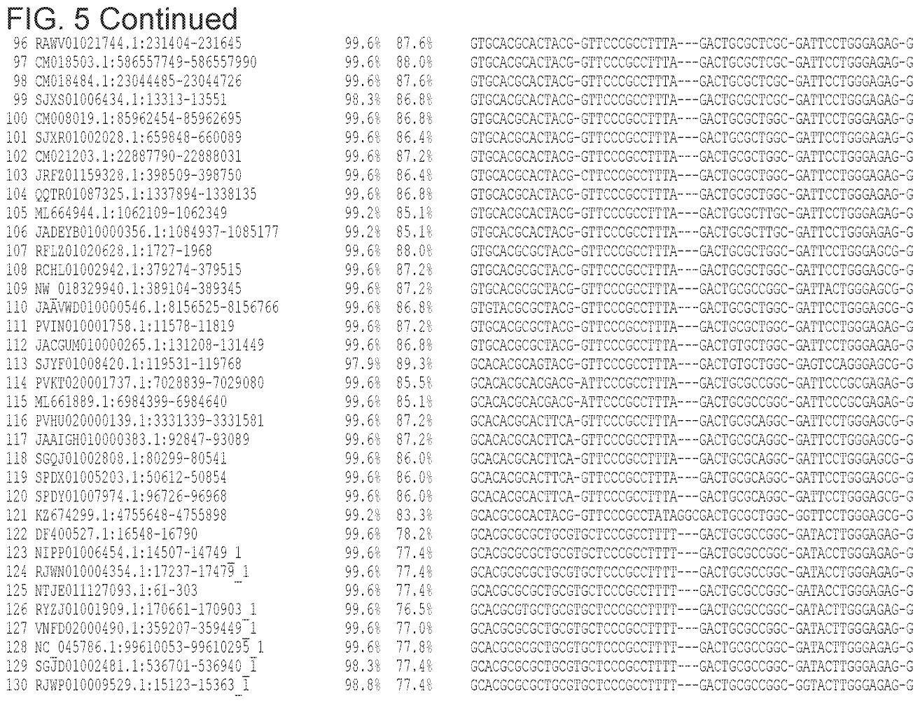

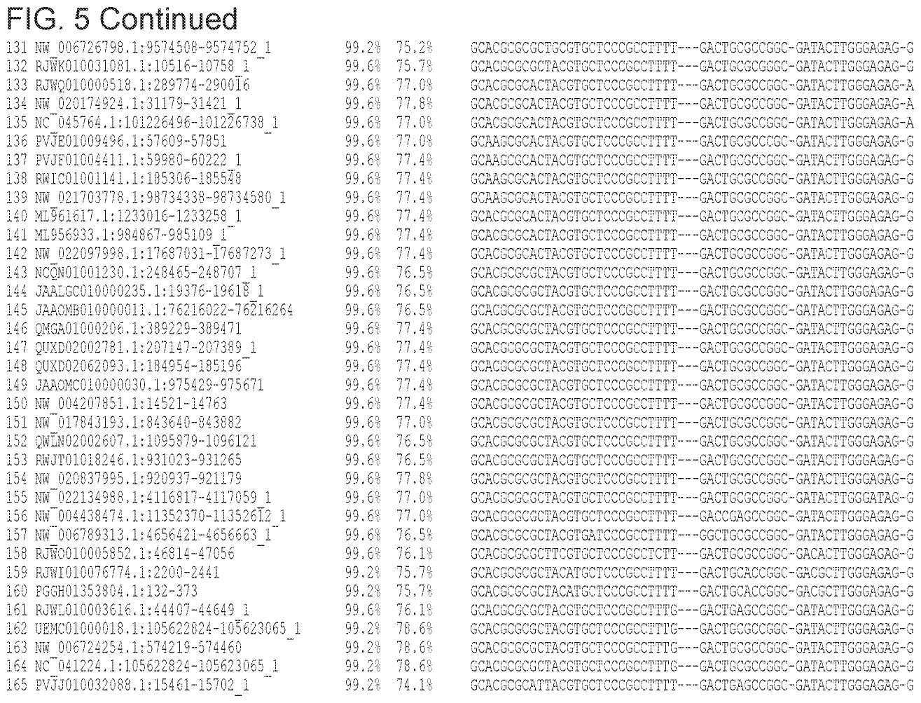

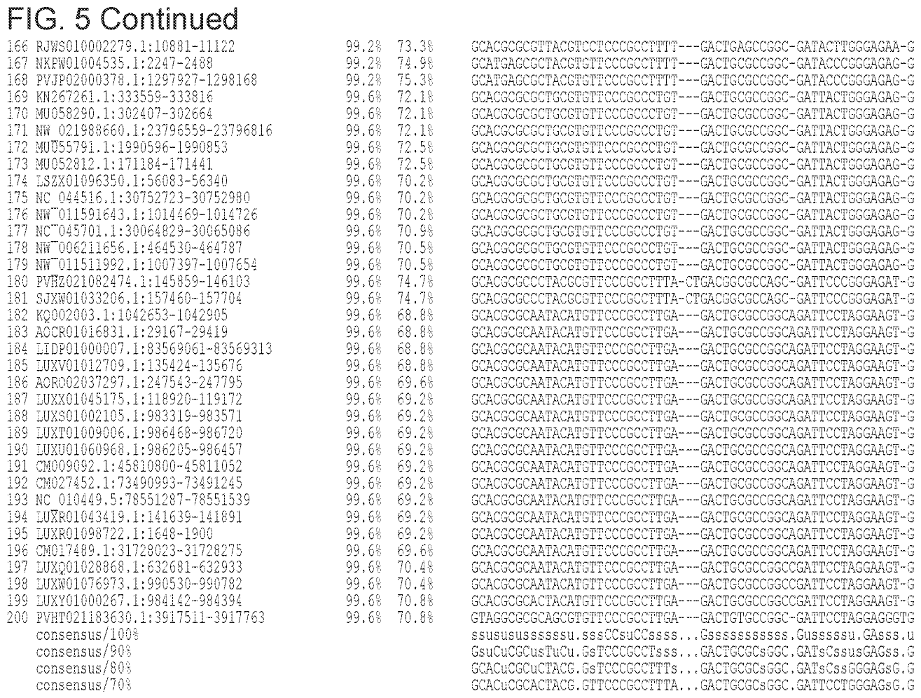

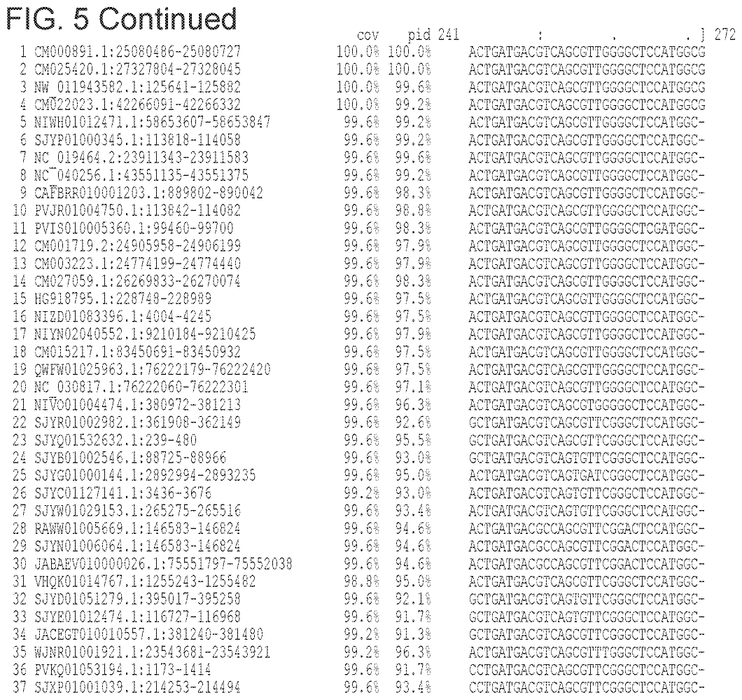

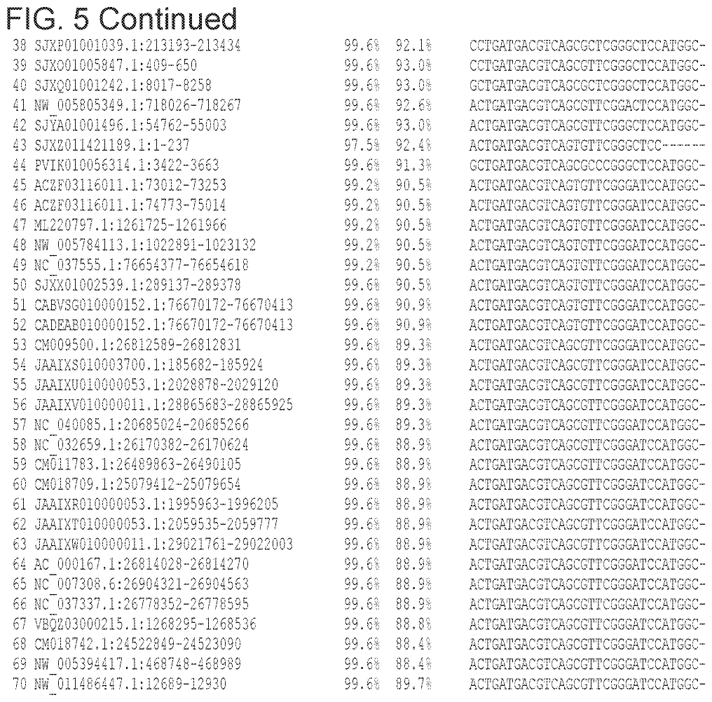

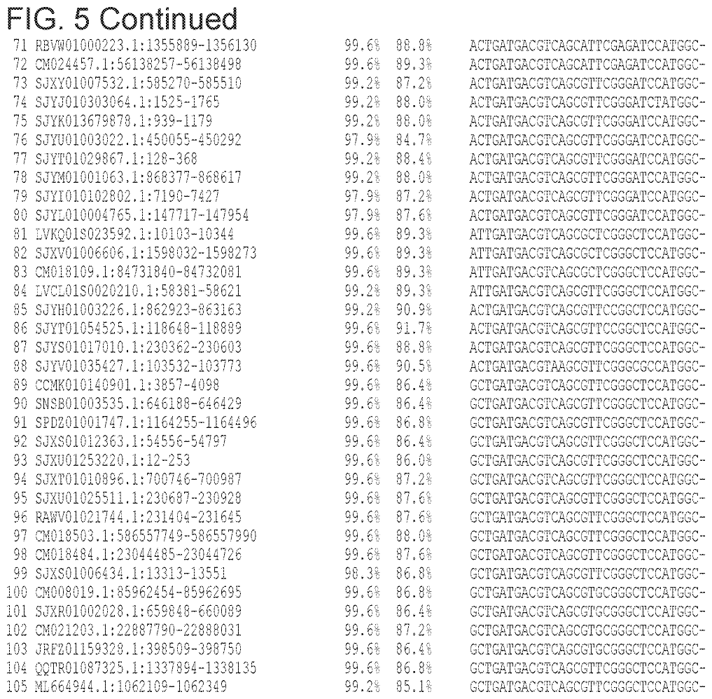

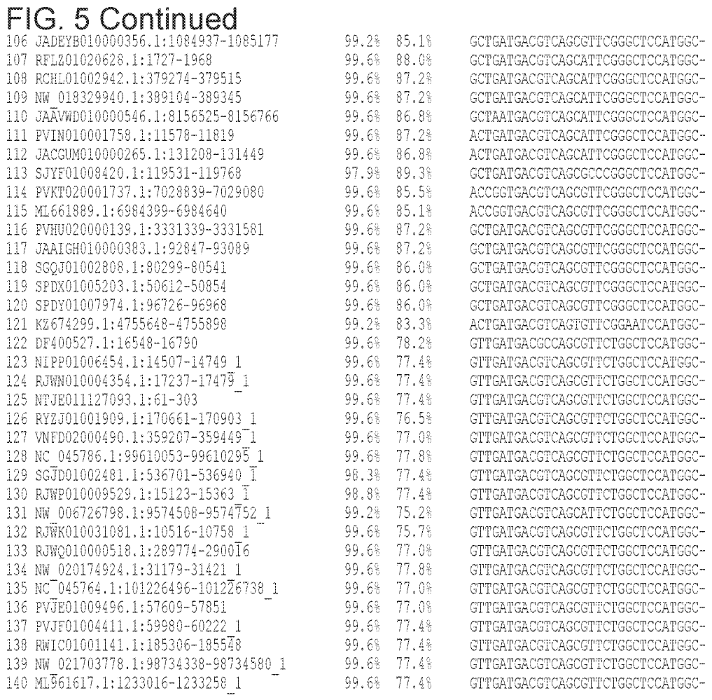

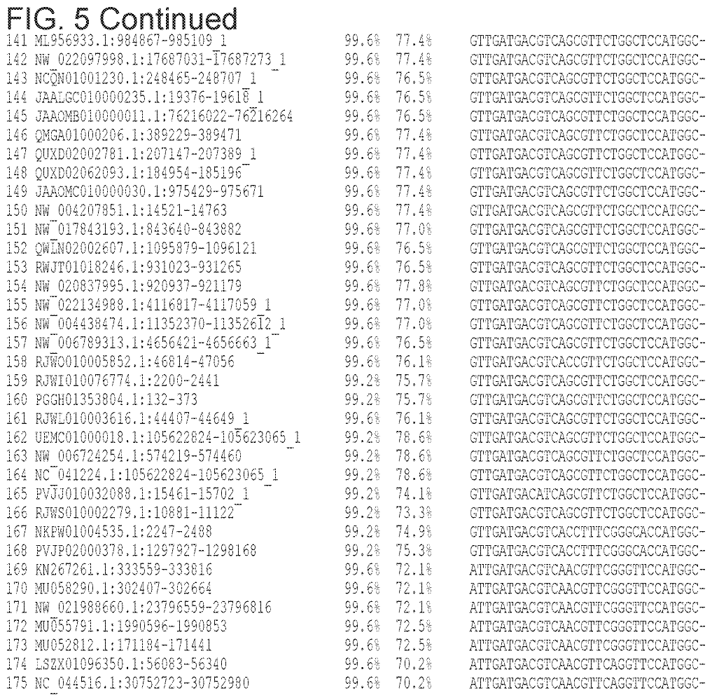

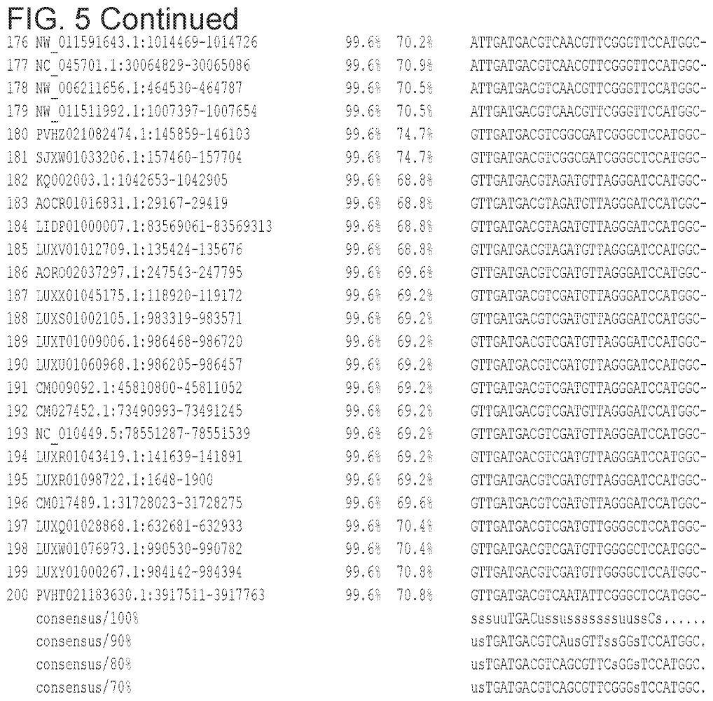

In certain embodiments, the promoter comprises an Artiodactyla H1 promoter. An alignment of Artiodactyla H1 promoter sequences is provided in FIG. 5 (wherein sequences numbered 1-200 in FIG. 5 correspond to SEQ ID NOs: 269-468 and consensus/100%, consensus/90%, consensus/80% and consensus/70% correspond to SEQ ID NOs 1811-1814, respectively). In some embodiments, the promoter comprises a nucleotide sequence having at least 85%, 90%, 95%, 96%, 97%, 98%, 99%, or 100% identity to nucleotides 20-266 of any one of the sequences in FIG. 5 or a functional fragment or variant (e.g., codon optimized) thereof.

In certain embodiments, the Artiodactyla H1 promoter comprises a sequence selected from the sequences in TABLE 5:

TABLE 5

| | |
|---|---|
| Artiodactyl | TGAGCTTCCCKCCGCCCTAYGSMRAAMAMYRSSCKCAARSMGCATTTATAA |
| Alignment | KGMKCYCAWACCTARAGMCAYTTKWCGGTTAYGGTGACTTCCCAYAASACA |
| consensus | TTGCGACATGCAAATAYTDYRGWGCGTYCCKCCCCTGGYARYTCCWCGCTR |
| sequence | GGACGCACRCGCRCTACGNGTTCCCGCCTTTWGACTGCGCYGGCGATTCCW |
| 75%_Identity | GGGAGMGGRYTGATGACGTCAGCGTTCGGGMTCCATGGCG (SEQ ID |
| | NO: 469) |
| Artiodactyl | TGAGCTTCCCKCCGCCCTAYGBMRRAVRVYDSSYKCARDSMRCAYTTATAA |
| Alignment | DGHKCYCADAMSTARAKMSAYTTBWCRSTTAYGGTGACTTCYCRYAASACA |
| consensus | TTGSGAYATGCAAATAYTDYRGWGCGTYNNNCCKCSCCTGGNYARYTYYWC |
| sequence | GCYRGGACGCACRCGCRCTRCGNGYTCCCGCCTTTWGACTGCGCYGGCGAT |
| 85%_Identity | WCYWGGGAGMGGRYTGATGACGTCARYGTTSKGGMTCCATGGCG (SEQ |
| | ID NO: 470) |
| Artiodactyl | TGAGCTTCCCKCCGCCCYAYRBVRRANRVYDVVYKCWRDBMRCRYTTATAA |
| Alignment | NRHKCYCADAMSTARAKHSAYTTBWYRSTTAYGGTGACTTCYCRYAASACA |
| consensus | KTGSGRYATGCAAATAYTDYRGHGYGYHNNNCCBCSYCYGGNNNNNYARYT |
| sequence | YYDCKCYRGGACGYRCRCGCRMTRCRNGYTCCCGCCTWKWGACTGCGCYGG |
| 90%_Identity | CGATWCYWRSGAGMKGRYTGATGACGTCARYGTTSKGGMTCCATGGCG |
| | (SEQ ID NO: 471) |
| Artiodactyl | TGAGCTTCYCKCCGCCCYAYRNNRRRNRNBDVVBBCWVNBMRYVYTTATAA |
| Alignment | NRHKCBCADAVBKARRKHVAYTTBWYRVTTAYGGYGAYTTCYCNRHAMSRC |
| consensus | AKWGSRRYATGCAAATAYKDYRGHNNNNNNGYRYHNNNCCBSBYCYRKNNN |
| sequence | NNNYADBTYYDCKNCYRGGACGYRSRCGCRMTRCRNGYTCCCGCCYWKWGA |
| 95%_Identity | CTGCGCYSGCNGATWMYHRNGARVKGRYTGATGACGTCRRYRTTVKGGHTC |
| | CATGGCG (SEQ ID NO: 472) |
| Artiodactyl | TGAGCTTCYCDCCGCCCYRYVNNVRNNNNBNNNNNBDVNNHRYVYTTATAA |
| Alignment | NRNDCBSRNRNBBNVRKNNAYNNNHHRVTTAYGGYGAYTYCYCNRHAMSVM |
| consensus | ABWGSRRBATGYAAATAYBNYRGHNNNNNNRBRYHNNNCCBSBYCHDDNNN |
| sequence | NNNHMDBKYYDHNNNNNGKACRYRNRCRYVVBNYRNSYTCCSGCCYWKDNN |
| 99%_Identity | GAYBGHRCHVGYNGRYWMYNRNGARVKRVYTGATGACGYMRVYRHKVNGRH |
| | WCCATGGCG (SEQ ID NO: 473) |
| Artiodactyl | TGAGCTYCYCDCCGCCYYRHNNNNNNNNNNNNNNNBNNNNNNVNNNRYNNT |
| Alignment | WATAWNRNDCBSRNVNNBNVRBNNAYNNNHHVNYTAYGGYGAYTYCYCNRH |
| consensus | AMSVVABWGSRNRBATGYAAATNNBNHRNHNNNNNNRBRBHNNNCSNNBYY |
| sequence | NDDNNNNNNNMDBBYBNNNNNNNRDRCVBRNRMRYVNNNHRNVHYCCSRCC |
| 100%_Identity | YHKDNNNGVYBBHNSNNSYNGRBDMYNRNGADVNNRVYYRRTGACRYMRVY |
| | DHBNNRRHDCBATGGCG (SEQ ID NO: 474) |

In some embodiments, the promoter comprises a nucleotide sequence having at least 85%, 90%, 95%, 96%, 97%, 98%, 99%, or 100% identity to nucleotides 20-238 of any one of SEQ ID NOs: 469-474 or a functional fragment or variant (e.g., codon optimized) thereof.

### Carnivora H1 Promoters

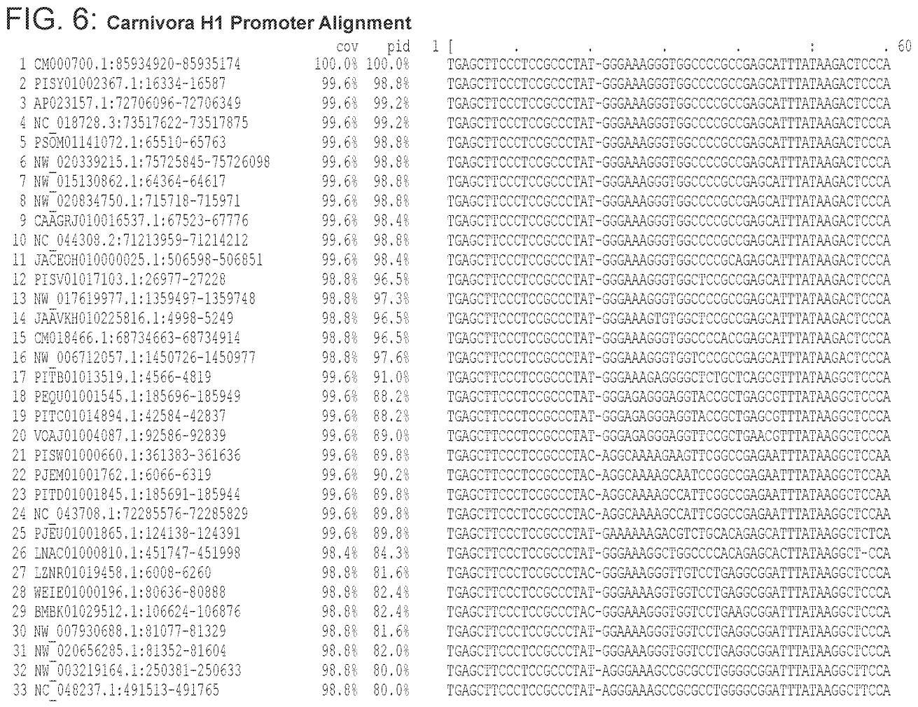

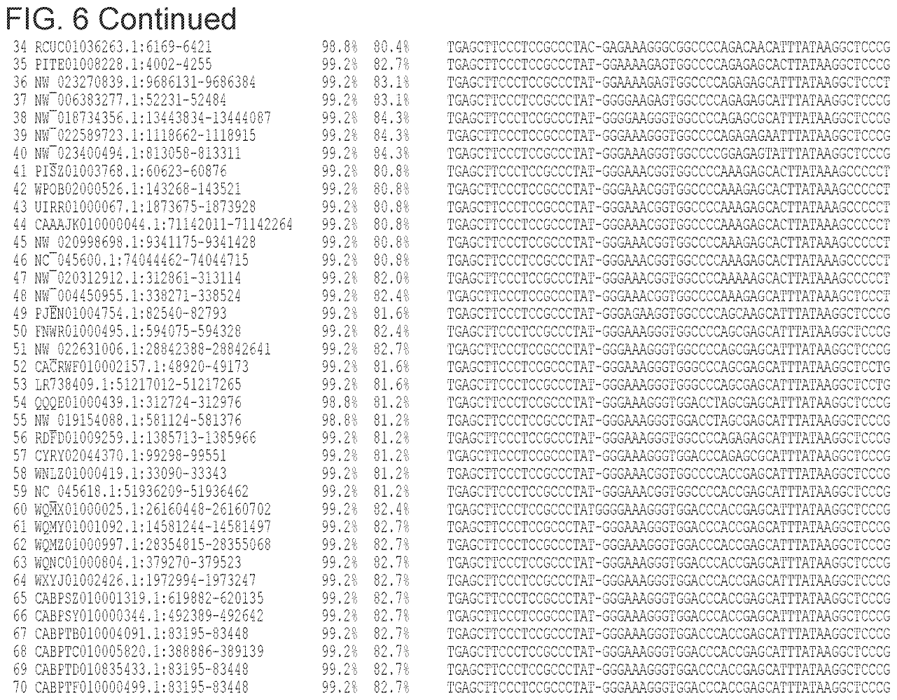

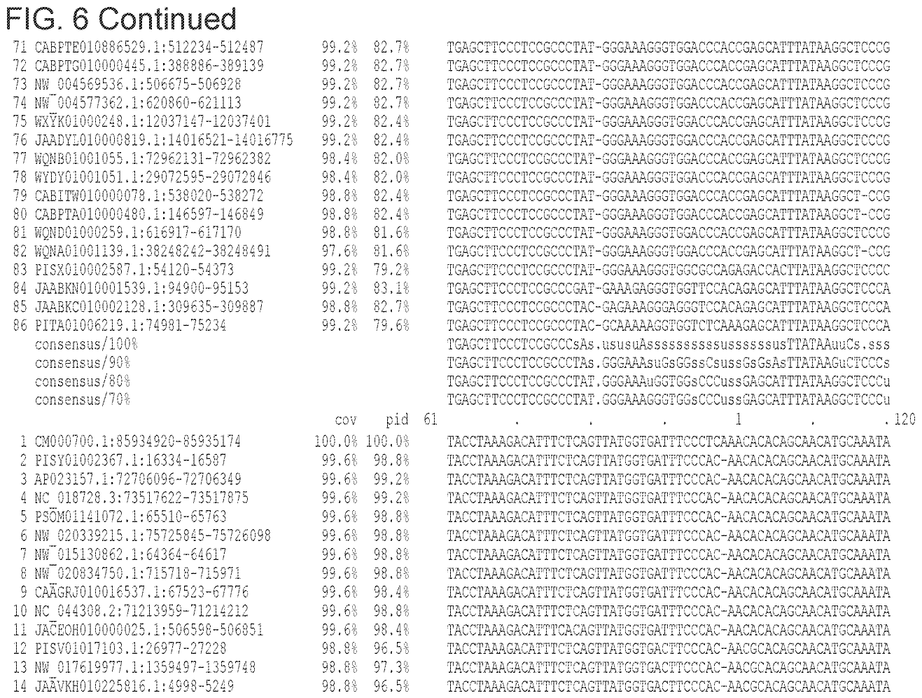

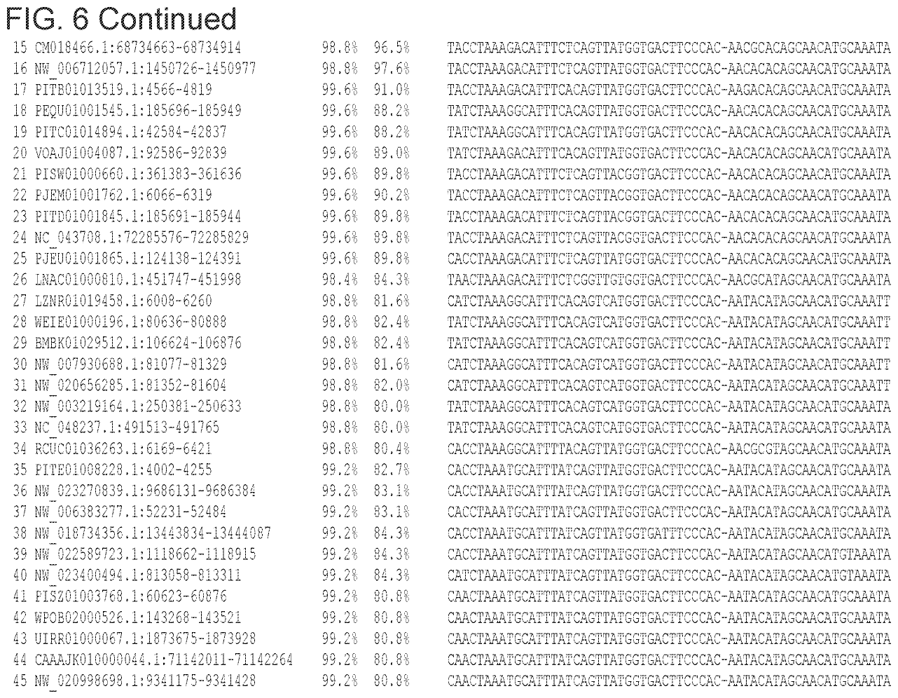

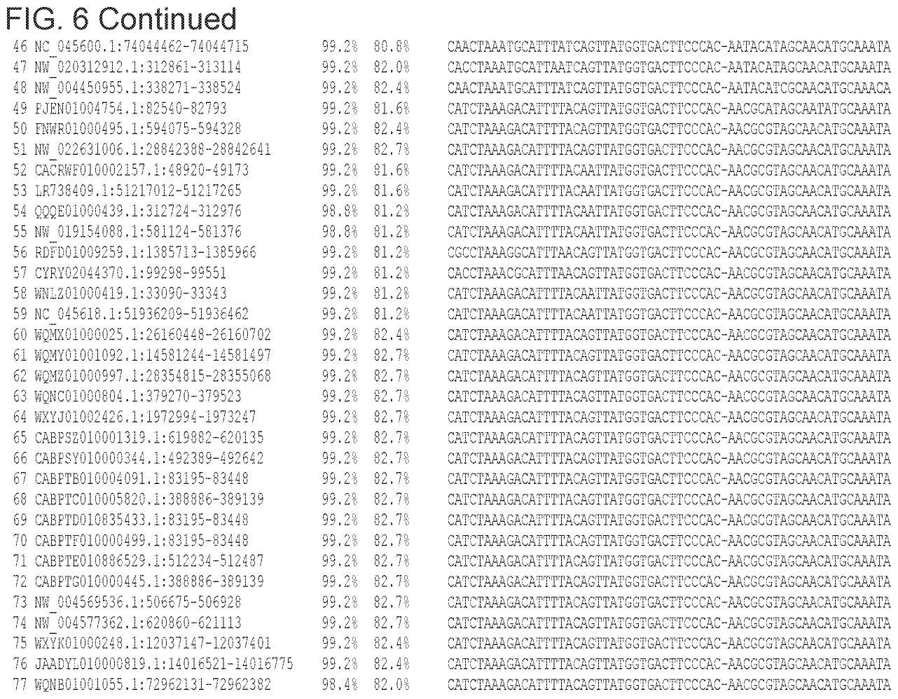

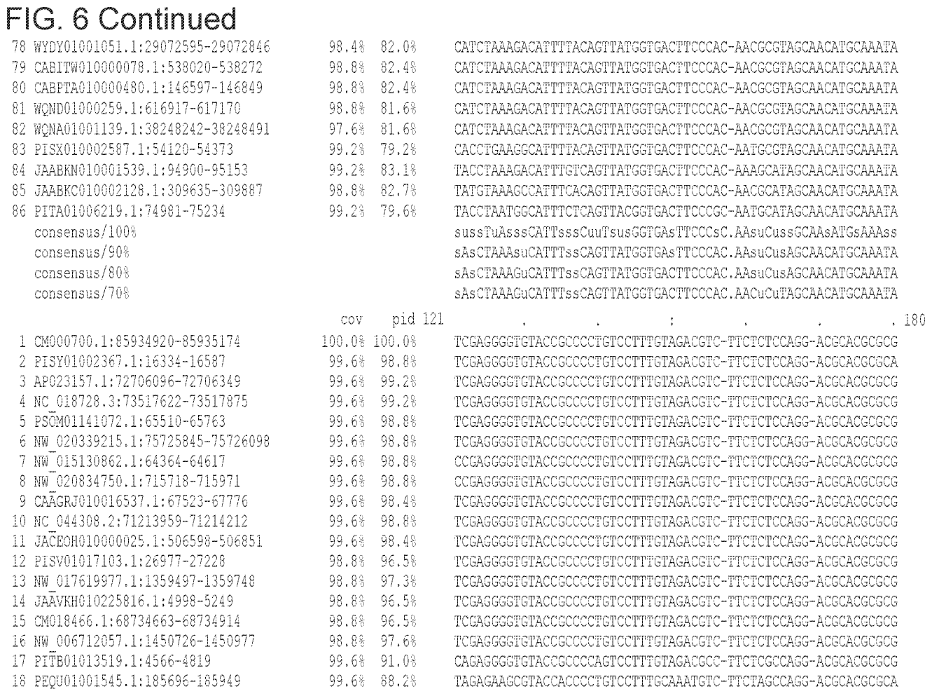

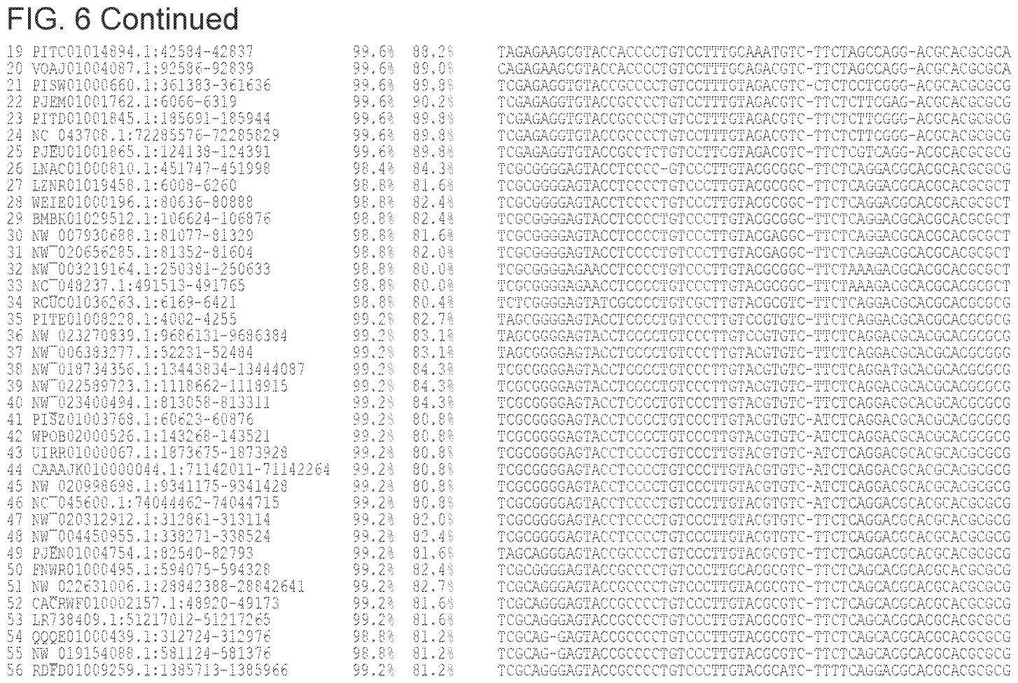

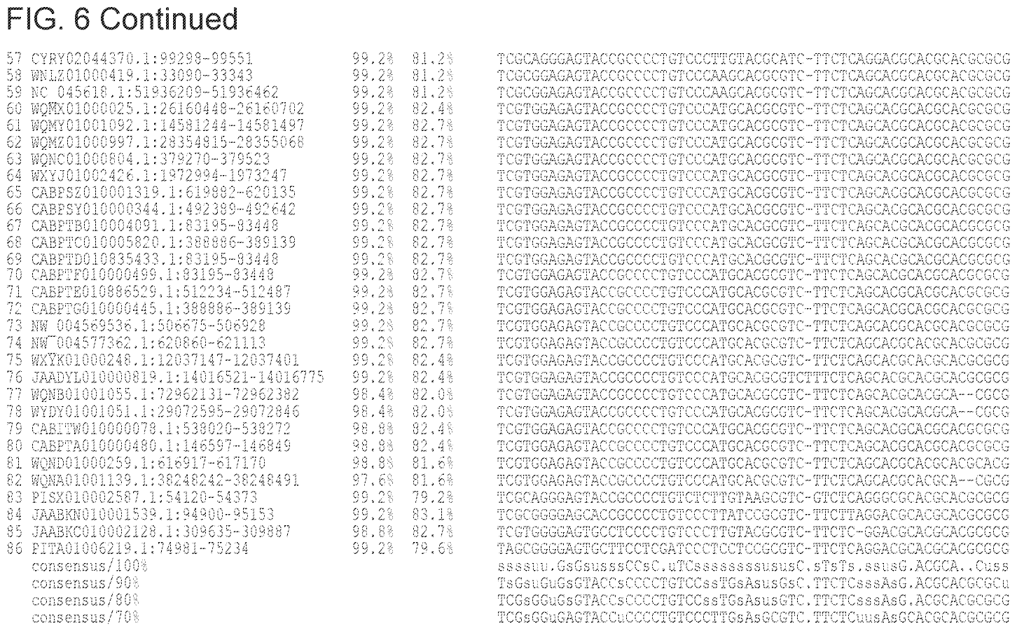

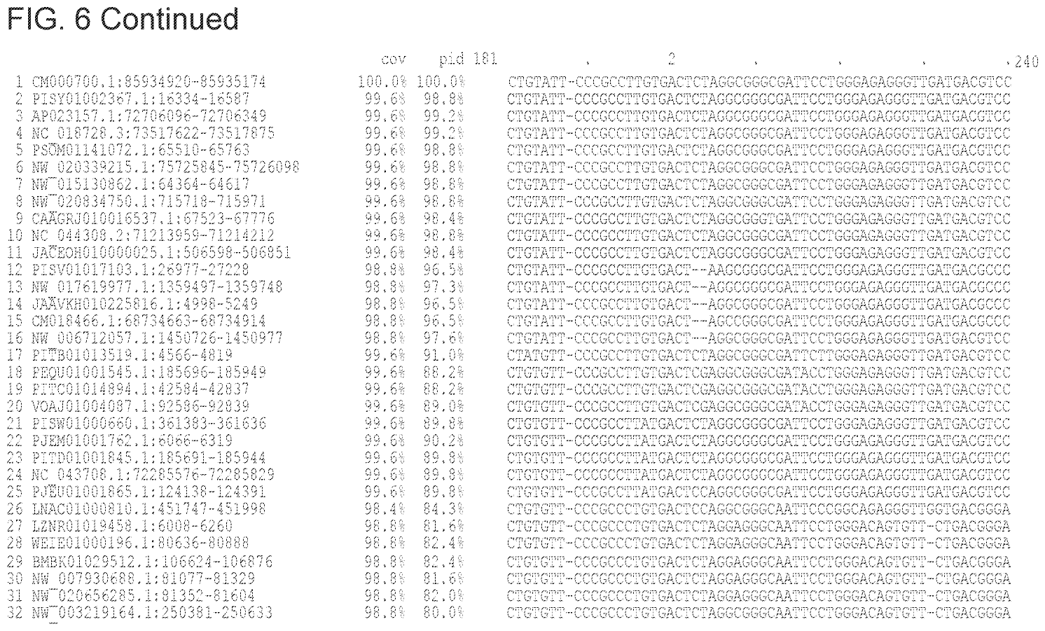

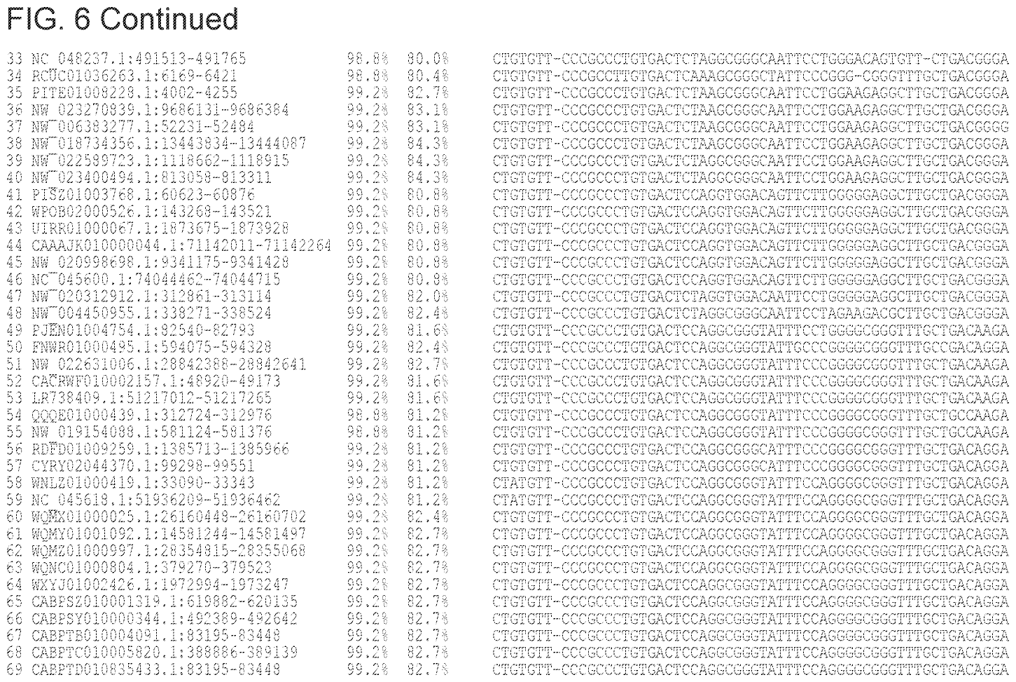

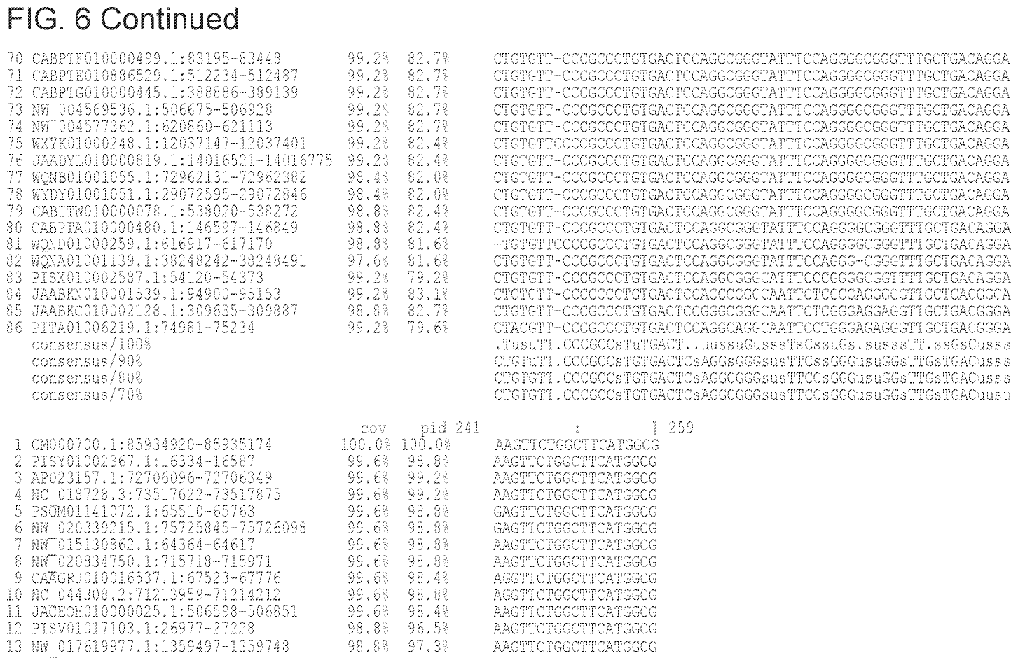

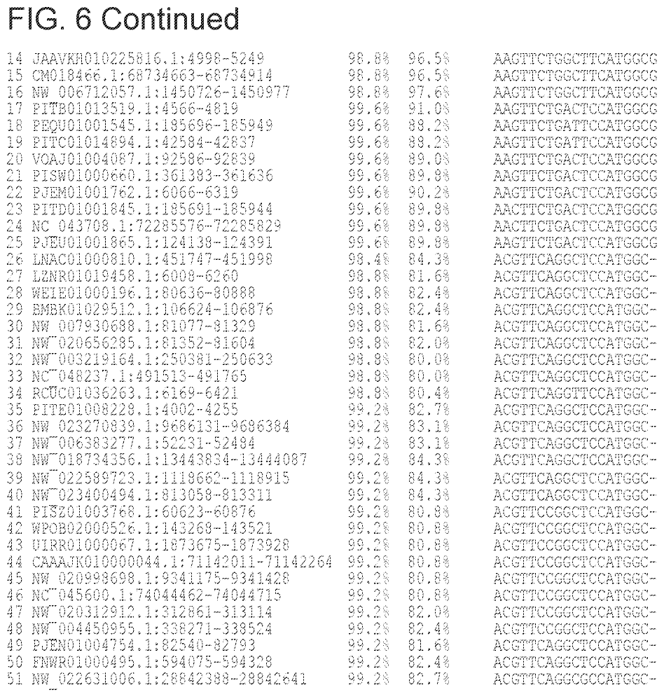

In certain embodiments, the promoter comprises a Carnivora H1 promoter. An alignment of Carnivora H1 promoter sequences is provided in FIG. 6 (wherein sequences numbered 1-86 in FIG. 6 correspond to SEQ ID NOs: 475-558 and SEQ ID NOs: 1809-1810, and consensus/100%, consensus/90%, consensus/80% and consensus/70% correspond to SEQ ID NOs: 1815-1818, respectively). In some embodiments, the promoter comprises a nucleotide sequence having at least 85%, 90%, 95%, 96%, 97%, 98%, 99%, or 100% identity to nucleotides 20 to 253 any one of the sequences in FIG. 6 or a functional fragment or variant (e.g., codon optimized) thereof.

In certain embodiments, the Carnivora H1 promoter comprises a sequence selected from those in TABLE 6.

TABLE 6

| | |
|---|---|
| Carnivora | TGAGCTTCCCTCCGCCCTATGGGGAAAGGGTGGMCCCRSMGAGCATTTATA |
| Alignment | AGGCTCCCRYAYCTAAAGRCATTTYWCAGTTATGGTGACTTCCCACAAAYR |
| consensus | CRYAGCAACATGCAAATATCGHGGRGWGTACCKCCCCTGTCCYWTGYASRC |
| sequence | GTCTTTCTCWSSASGCACGCACGCGCGCTGTGTTCCCCGCCYTGTGACTCY |
| 75%_Identity | AGGCGGGYRWTTCCWGGGRSRGGKTTGMTGACRKSMAMGTTCWGGCTYCAT |
| | GGCG (SEQ ID NO: 559) |
| Carnivora | TGAGCTTCCCTCCGCCCTATGGGGAAAVGGYGGHYCYRVMGAGSATTTATA |
| Alignment | AGRCTCCCRYAYCTAAAKRCATTTHWCAGTTATGGTGACTTCCCACAAAYR |
| consensus | CRYAGCAACATGCAAATATCGHGGRGWGTACCKCCCCTGTCCYWTGYASRY |
| sequence | GTCTTTCTCWSSASGCACGCACGCGCGCTGTRTTCCCCGCCYTGTGACTCY |
| 85%_Identity | AGGCGGGYRWTTCCHGGGRSRGGBTTGMTGACRKSMAMGTTCWGGCTYCAT |
| | GGCG (SEQ ID NO: 560) |
| Carnivora | TGAGCTTCCCTCCGCCCTAYGGGGAAAVRGYGGHYCYRVVGMGSAYTTATA |
| Alignment | AGRCTCCCDYAYCTAAAKRCATTTHWCAGTTATGGTGAYTTCCCACAAAYR |
| consensus | CRYAGCAACATGCAAATATMGHRGRGWGTACCKCCCCTGTCCYWTGYASRY |
| sequence | GKCTTTCTCWSSASGCACGCACGCGCKCTGTRTTCCCCGCCYTGTGACTCY |
| 90%_Identity | AGGYGGGYRWTTCYHGGGRSRGGBTTGMTGACRDSMAMGTTCWGRCTYCAT |
| | GGCG (SEQ ID NO: 561) |
| Carnivora | TGAGCTTCCCTCCGCCCTAYGRRRVRAVRGHVRNYCYRVVGMGVAYTTATA |
| Alignment | ARRCYCCMDYAHCTAAAKRCATTTHWCARTYAYGGTGAYTTCCCACAAAYR |
| consensus | CRYAGCAACATGCAAATWTMGHRRRGWGTACCKCCCCTGTCCYWTGYASRY |
| sequence | GKCTWTCTMDBSRSGCACGCACGCGCKCTGTRTTCCCCGCCYTRTGACTCY |
| 95%_Identity | ARGHGGRYRDTTCYHGGRRSRGKBTTGMTGACRDSMAMGTTCHGRCTYCAT |
| | GGCG (SEQ ID NO: 562) |
| Carnivora | TGAGCTTCCCTCCGCCCKAYGRVRVRAVDVNNNNNBBRVNVMVNRYTTATA |
| Alignment | ARRCYYYHNYRHSTRAWBVCATTWNWCRRTYRYGGTGAYTTCCCDCAAANR |
| consensus | CRYMGCAAYATGYAAAYWYMKHRRRGHGHRYYDCCYCDRTCBYWHVYMVRH |
| sequence | RBCTNTYTHNNSRNGCACGCACGCRSDCTRYRTTCCCCGCCYTRTGACTCN |
| 99%_Identity | RRSHRGRYDDTDCYHRGVRSRVKBTTGVYGMCRNSVRVBTYCHGRYKYCAT |
| | GGCG (SEQ ID NO: 563) |
| Carnivora | TGAGCTTCCCTCCGCCCKAYGRVRVRAVDVNNNNNBBRVNVMVNRYTTATA |
| Alignment | ARRCYYYHNYRHSTRAWBVCATTWNWCRRTYRYGGTGAYTTCCCDCAAANR |
| consensus | CRYMGCAAYATGYAAAYWYMKHRRRGHGHRYYDCCYCDRTCBYWHVYMVRH |
| sequence | RBCTNTYTHNNSRNGCACGCACGCRSDCTRYRTTCCCCGCCYTRTGACTCN |
| 100%_Identity | RRSHRGRYDDTDCYHRGVRSRVKBTTGVYGMCRNSVRVBTYCHGRYKYCAT |
| | GGCG (SEQ ID NO: 564) |

In some embodiments, the promoter comprises a nucleotide sequence having at least 85%, 90%, 95%, 96%, 97%, 98%, 99%, or 1000% identity to nucleotides 20-253 of any one of SEQ ID NOs: 559-564 or a functional fragment or variant (e.g., codon optimized) thereof.

### Cetacea H1 Promoters

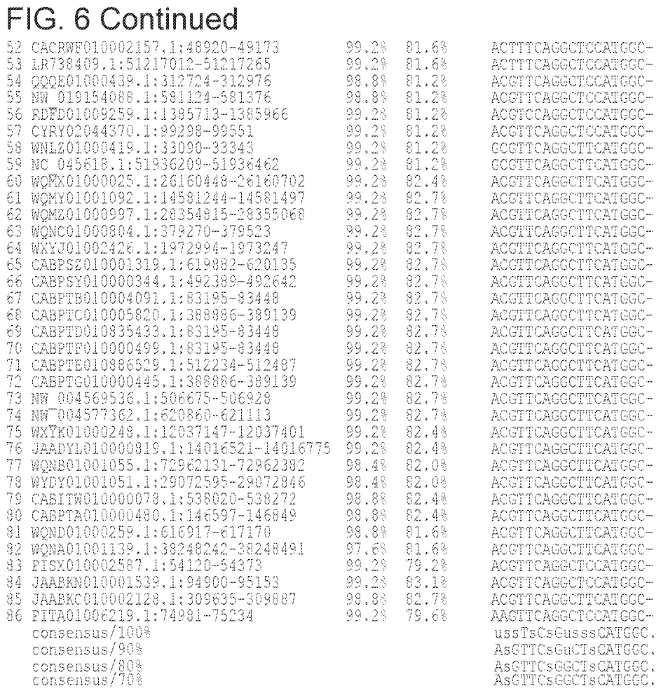

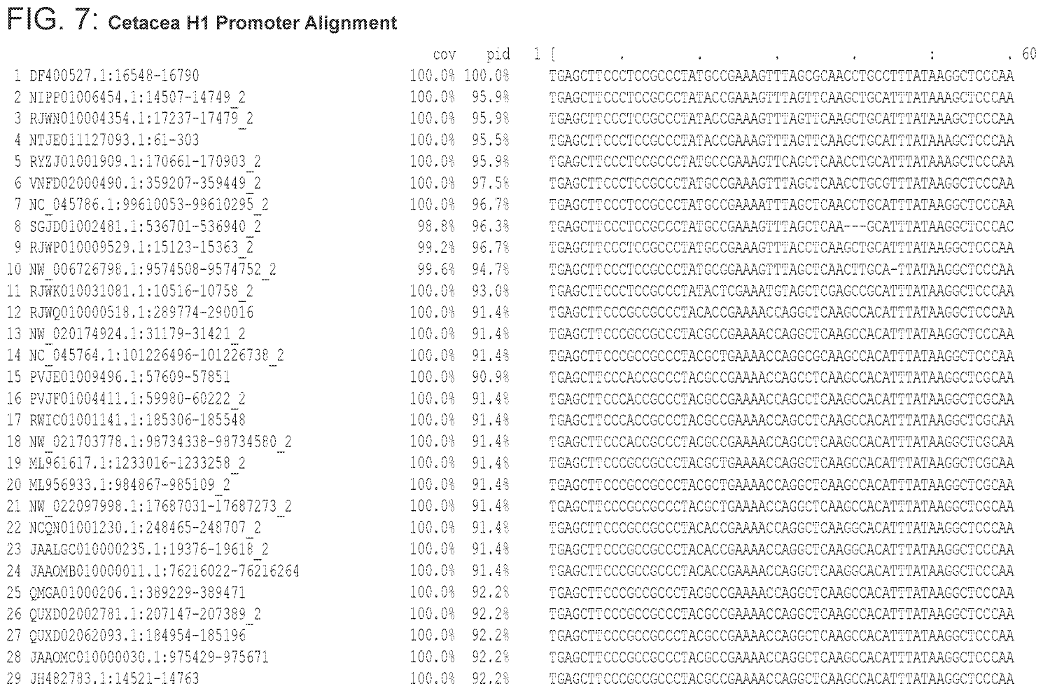

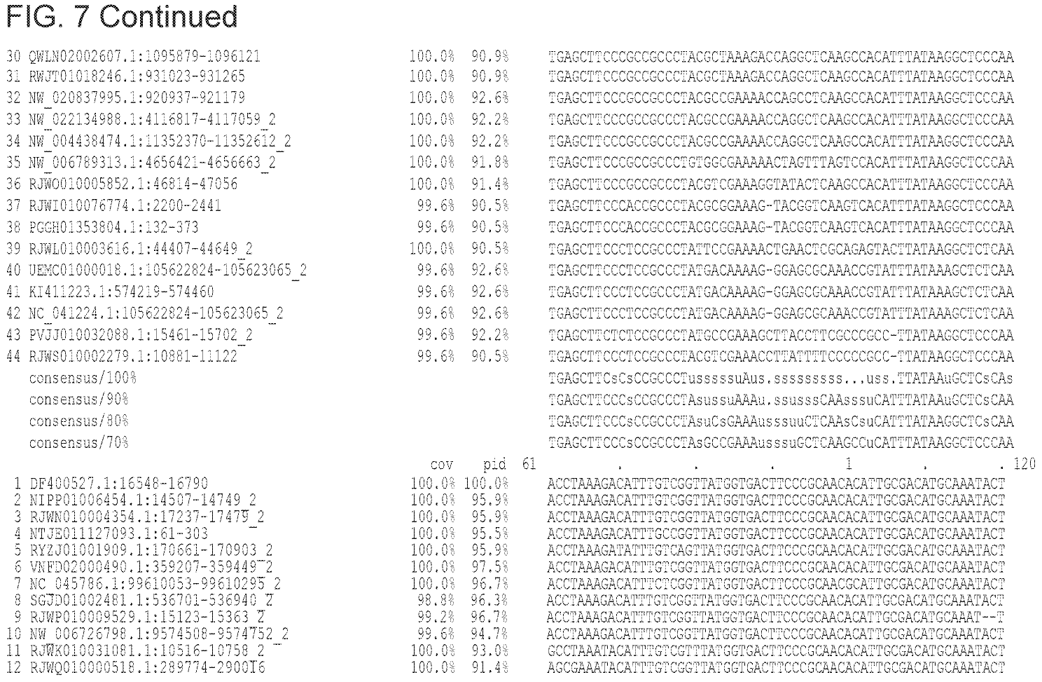

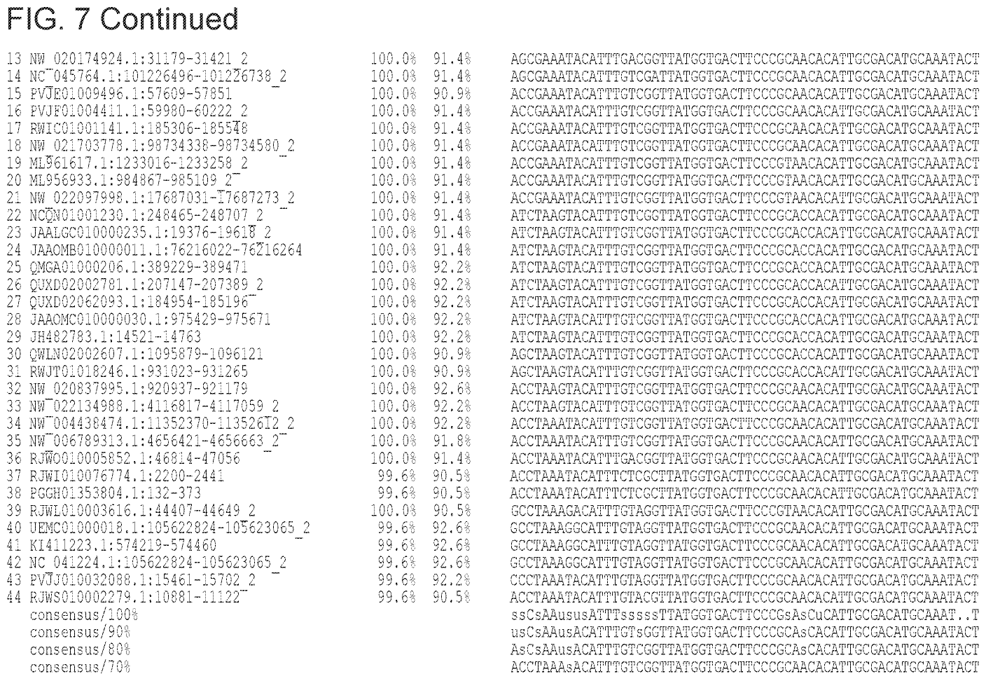

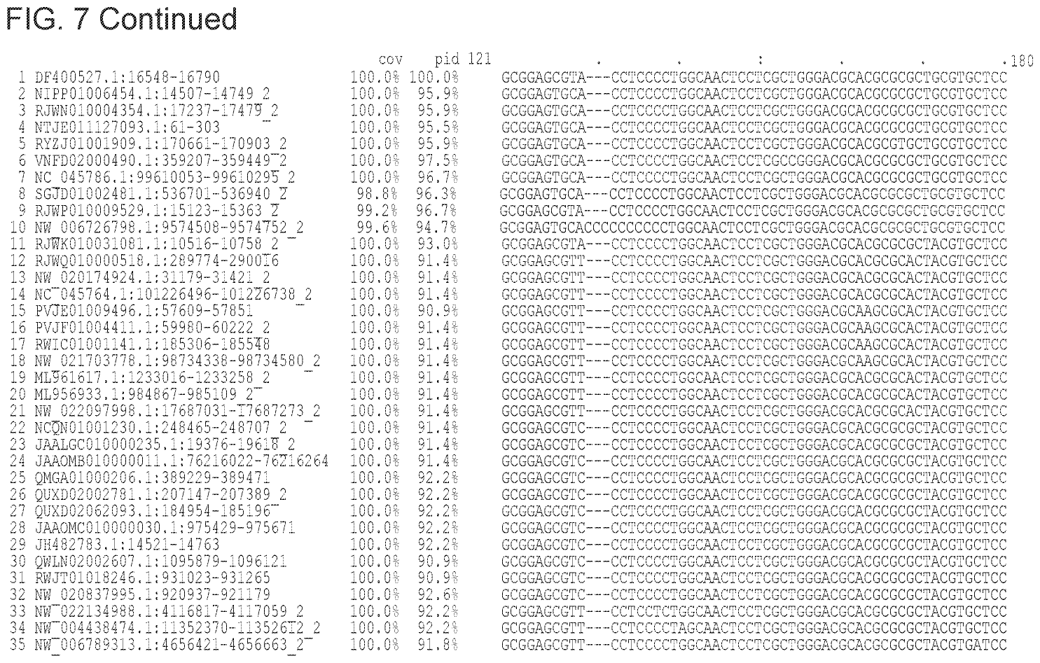

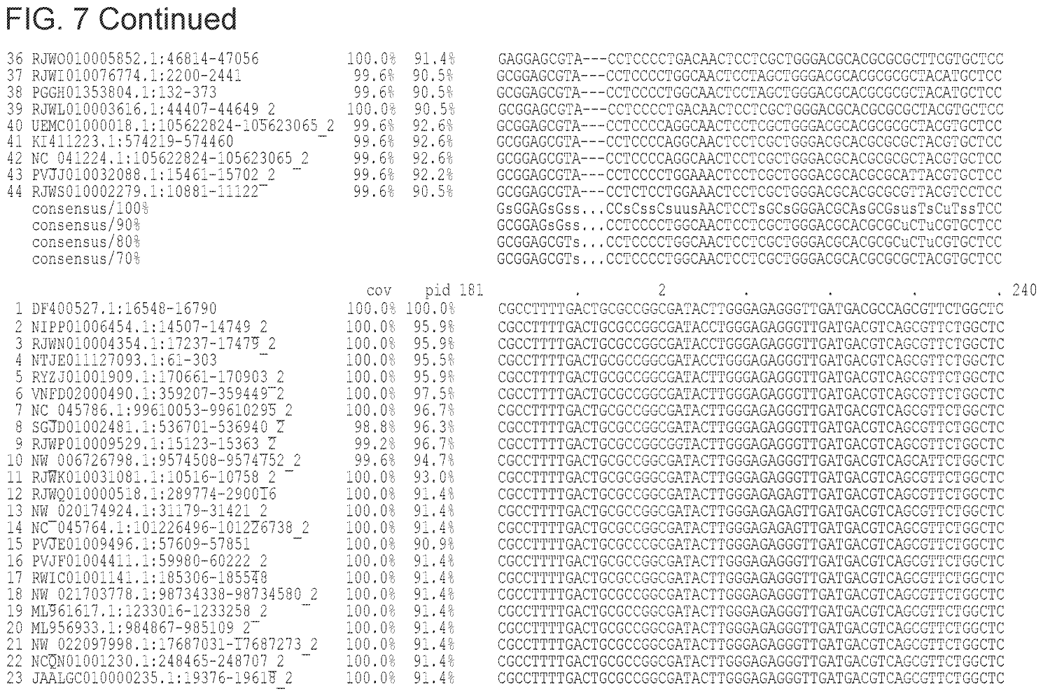

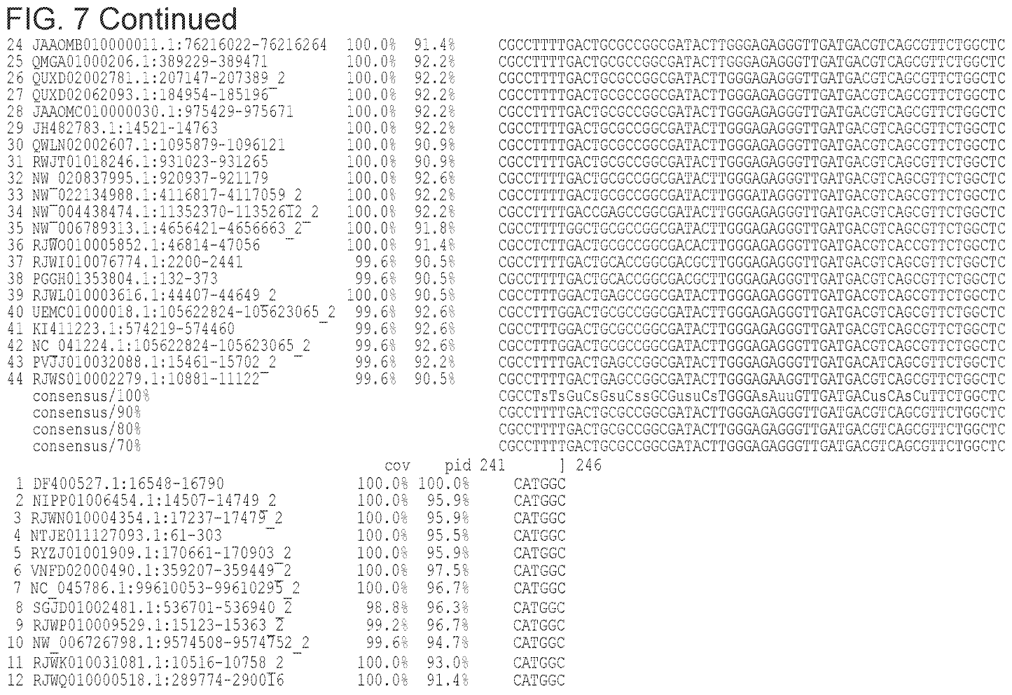

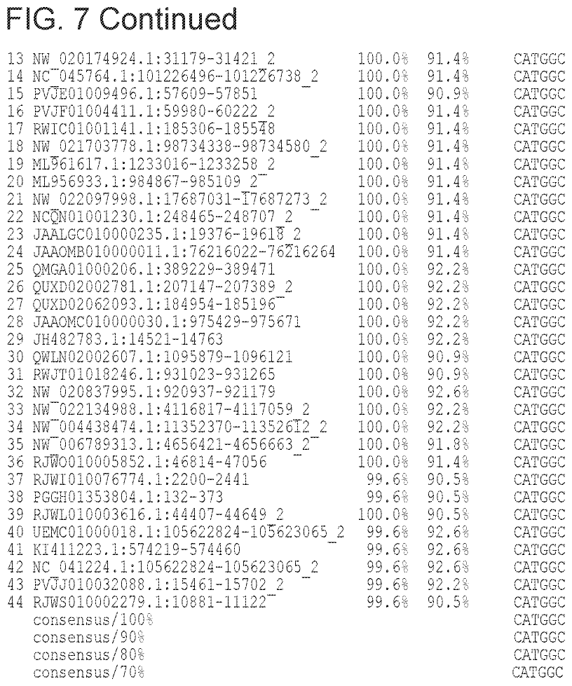

In certain embodiments, the promoter comprises a Cetacea H1 promoter. An alignment of Cetacea H1 promoter sequences is provided in FIG. 7 (wherein sequences numbered 1-44 in FIG. 7 correspond to SEQ ID NOs: 565-608, and consensus/100%, consensus/90%, consensus/80% and consensus/70% correspond to SEQ ID NOs: 1819-1822, respectively). In some embodiments, the promoter comprises a nucleotide sequence having at least 85%, 90%, 95%, 96%, 97%, 98%, 99%, or 100% identity to nucleotides 20-241 of any one of the sequences in FIG. 7 or a functional fragment or variant (e.g., codon optimized) thereof.

In certain embodiments, the Cetacea H1 promoter comprises a sequence selected from those in TABLE 7.

TABLE 7

| | |
|---|---|
| Cetacea | TGAGCTTCCCKCCGCCCTAYGCCGAAARYYWRGCTCAASCCRCATTTATAA |
| Alignment | GGCTCCCAAAYCTAARKACATTTGTCGGTTATGGTGACTTCCCGCAACACA |
| consensus | TTGCGACATGCAAATACTGCGGAGCGTWCCTCCCCTGGCAACTCCTCGCTG |
| sequence | GGACGCACGCGCGCTACGTGCTCCCGCCTTTTGACTGCGCCGGCGATACTT |
| 75%_Identity | GGGAGAGGGTTGATGACGTCAGCGTTCTGGCTCCATGGC (SEQ ID NO: |
| | 609) |
| Cetacea | TGAGCTTCCCKCCGCCCTAYRCYGAAARNYWRSYTCAASSYRCATTTATAA |
| Alignment | RGCTCSCAAAYCKAARKACATTTGTCGGTTATGGTGACTTCCCGCAMCACA |
| consensus | TTGCGACATGCAAATACTGCGGAGYGYHCCTCCCCTGGCAACTCCTCGCTG |
| sequence | GGACGCACGCGCRCTRCGTGCTCCCGCCTTTTGACTGCGCCGGCGATACTT |
| 85%_Identity | GGGAGAGGGTTGATGACGTCAGCGTTCTGGCTCCATGGC (SEQ ID NO: |
| | 610) |
| Cetacea | TGAGCTTCCCDCCGCCCTAYRMYRAAARNYDRSYKCAAVSYRCATTTATAA |
| Alignment | RGCTCSCAARBCKAARKACATTTGTMGGTTATGGTGACTTCCCGCAMCACA |
| consensus | TTGCGACATGCAAATACTGCGGAGYGYHCCTCCCCTGGCAACTCCTCGCTG |
| sequence | GGACGCACGCGCRCTRCGTGCTCCCGCCTTTTGACTGCGCCGGCGATACTT |
| 90%_Identity | GGGAGAGGGTTGATGACGTCAGCGTTCTGGCTCCATGGC (SEQ ID NO: |
| | 611) |
| Cetacea | TGAGCTTCCCDCCGCCCTAYRHBRAAARNBDVVYKYVVVBYRYMNTTATAA |
| Alignment | RGCTCBCAARBCKAARKRCATTTSWMGSTTATGGTGACTTCCCGYAMCACA |
| consensus | TTGCGACATGCAAATACTGCGGAGYGYHCCTCCCCWGGCAACTCCTCGCTG |
| sequence | GGACGCAMGCGCRCTRCGTGCTCCCGCCTTTKGACTGMGCCGGCGAYACYT |
| 95%_Identity | GGGAGAGRGTTGATGACGTCAGCGTTCTGGCTCCATGGC (SEQ ID NO: |
| | 612) |
| Cetacea | TGAGCTTCYCDCCGCCCTRYDNBVRARVNBNNNBKYVVNNNRYVNTTATAA |
| Alignment | RGCTCBCAMVBCKAARKRYATTTSHMVNTTATGGTGACTTCCCGYAMCRCA |
| consensus | TTGCGACATGCAAATNNTGMGGAGYGYHNNNCCYCYYCWRRMAACTCCTMG |
| sequence | CYGGGACGCAMGCGYRYTDCRTSMTCCCGCCTYTKGRCYGMRCSSGCGRYR |
| 99%_Identity | CYTGGGAKARRGTTGATGACRYCASCRTTCTGGCTCCATGGC (SEQ ID |
| | NO: 613) |
| Cetacea | TGAGCTTCYCDCCGCCCTRYDNBVRARVNBNNNBKYVVNNNRYVNTTATAA |
| Alignment | RGCTCBCAMVBCKAARKRYATTTSHMVNTTATGGTGACTTCCCGYAMCRCA |
| consensus | TTGCGACATGCAAATNNTGMGGAGYGYHNNNCCYCYYCWRRMAACTCCTMG |
| sequence | CYGGGACGCAMGCGYRYTDCRTSMTCCCGCCTYTKGRCYGMRCSSGCGRYR |
| 100%_Identity | CYTGGGAKARRGTTGATGACRYCASCRTTCTGGCTCCATGGC (SEQ ID |
| | NO: 614) |

In some embodiments, the promoter comprises a nucleotide sequence having at least 85%, 90%, 95%, 96%, 97%, 98%, 99%, or 100% identity to nucleotides 20-238 ofany one of SEQ ID NOs: 609-614 or a functional fragment or variant (e.g., codon optimized) thereof.

### Chiroptera H1 Promoters

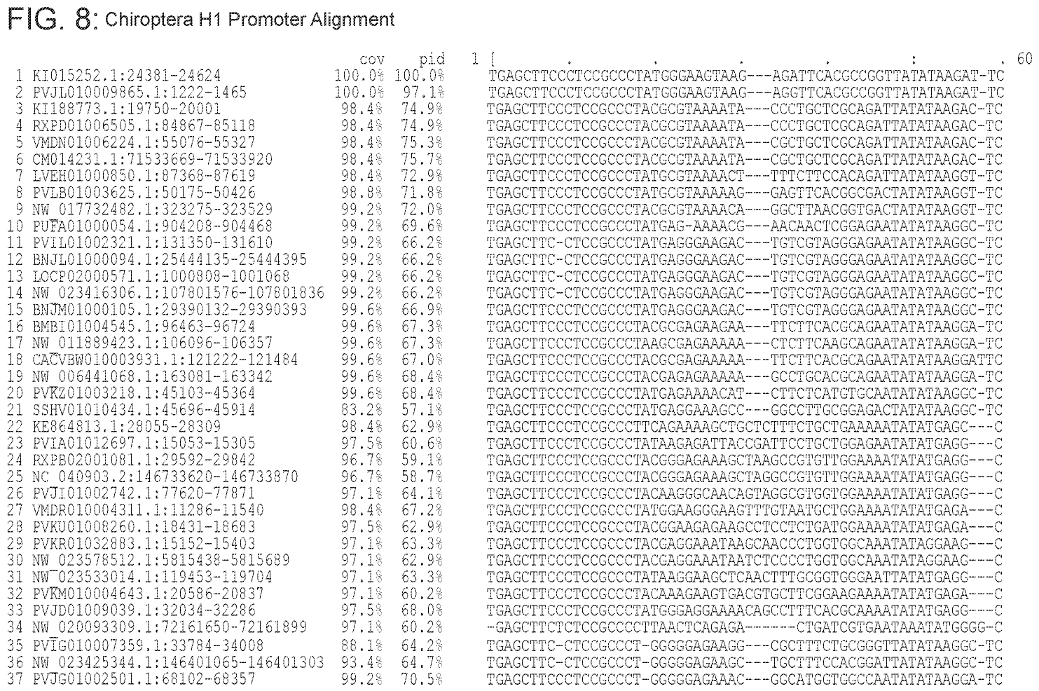

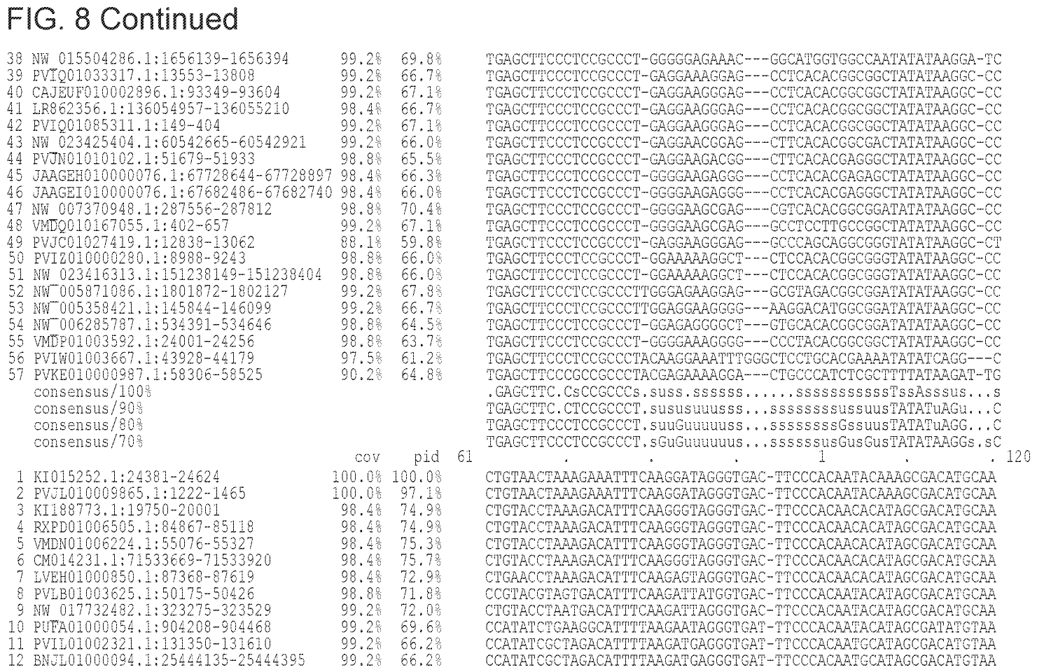

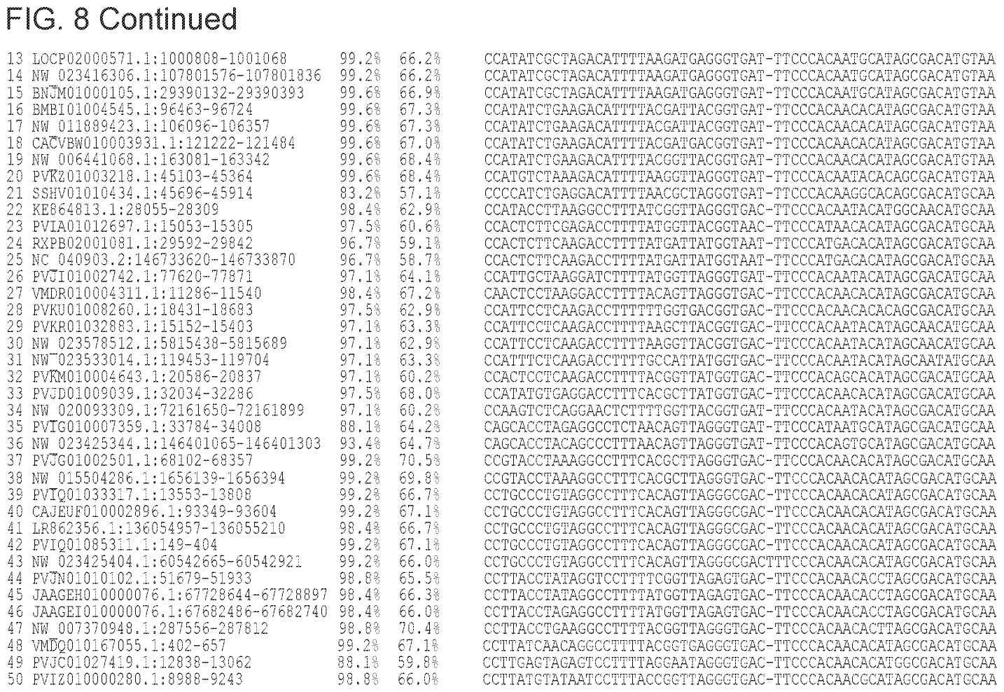

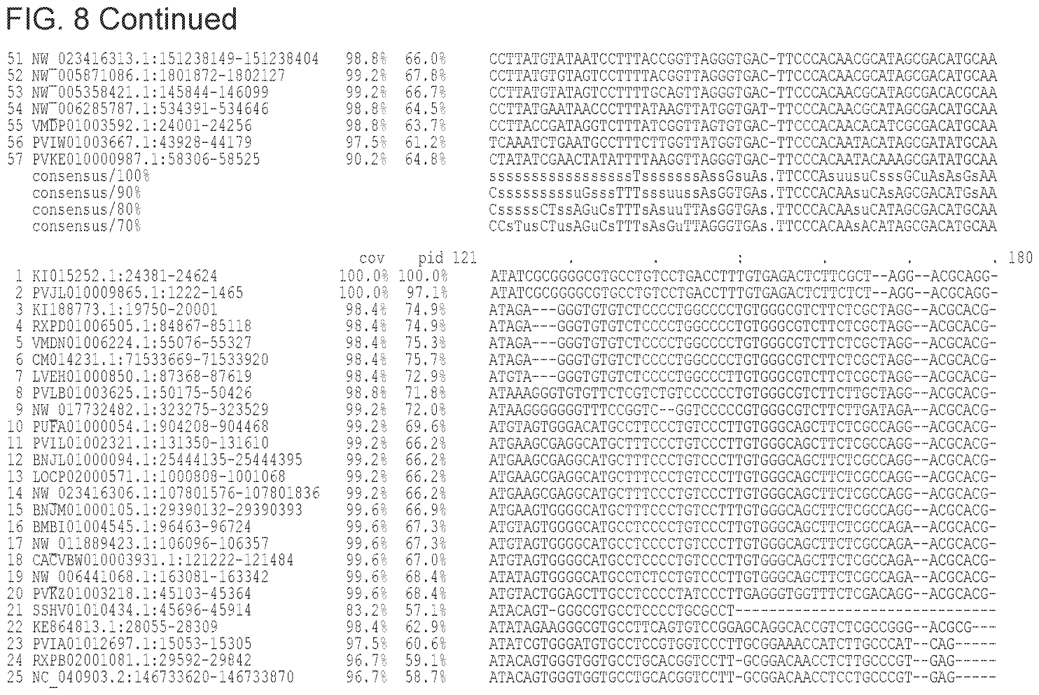

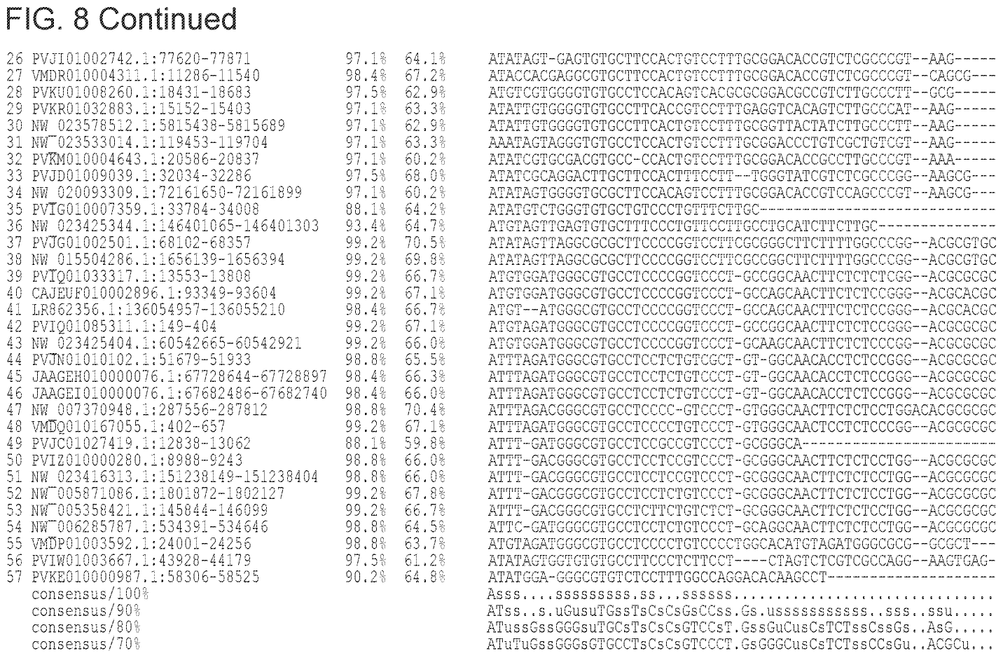

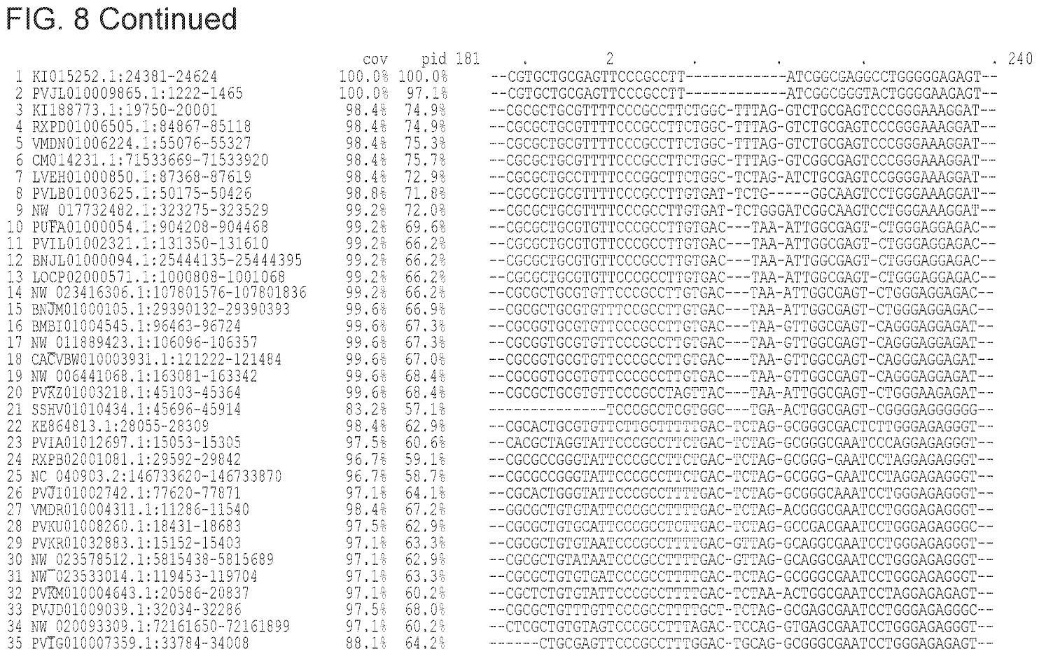

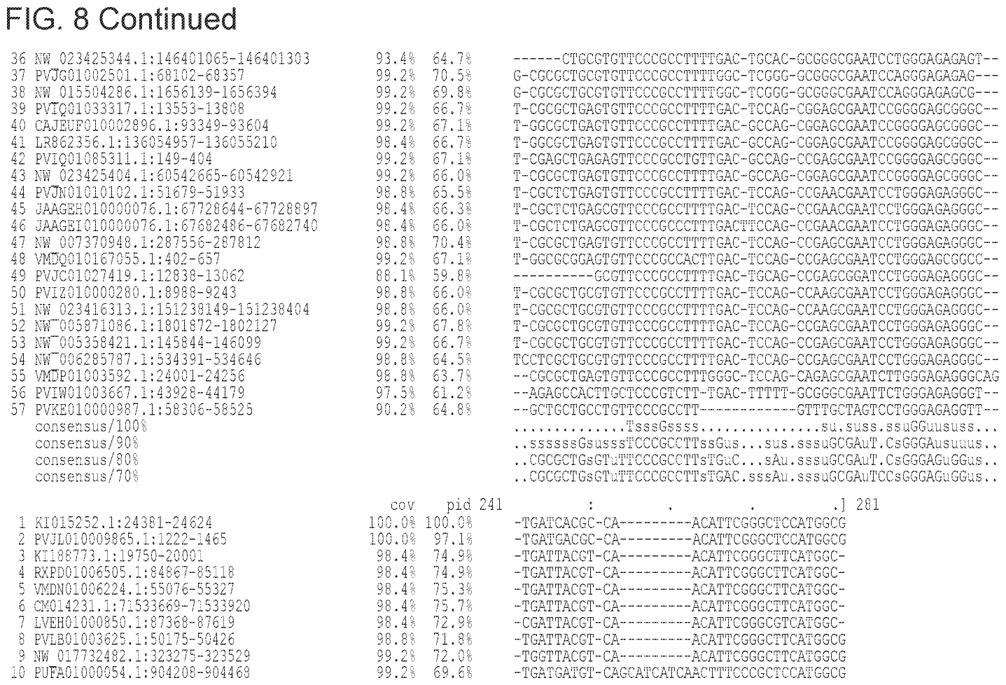

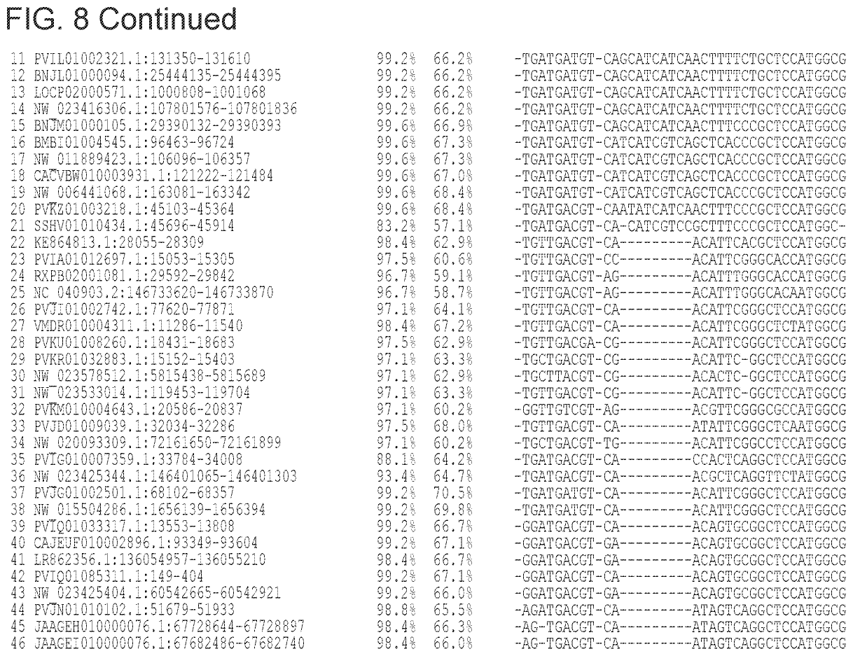

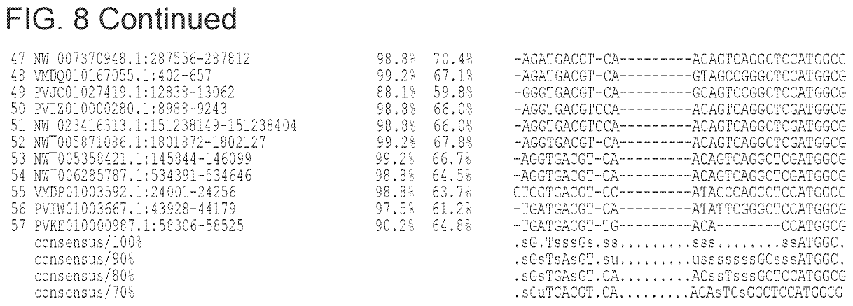

In certain embodiments, the promoter comprises a Chiroptera H1 promoter. An alignment of Chiroptera H1 promoter sequences is provided in FIG. 8 (wherein sequences numbered 1-57 in FIG. 8 correspond to SEQ ID NOs: 615-671, and consensus/100Y, consensus/90%, consensus/80% and consensus/70% correspond to SEQ ID NOs: 1823-1826, respectively). In some embodiments, the promoter comprises a nucleotide sequence having at least 85%, 90%, 95%, 96%, 97%, 98%, 99%, or 100% identity to nucleotides 20-276 of any one of the sequences in FIG. 8 or a functional fragment or variant (e.g., codon optimized) thereof.

In certain embodiments, the Chiroptera H1 promoter comprises a sequence selected from those in TABLE 8.

TABLE 8

| | |
|---|---|
| Chiroptera | TGAGCTTCCCTCCGCCCTNBGRGRRRRRVVBBYYWSNYGSMRRMTATATAA |
| Alignment | GGNYCCCWYWYCTVWAGRCMTTTYAMGRTTASGGTGAYTTCCCACAAYACA |
| consensus | TAGCGACATGCAAATRWNGHNGGGYGTGCCTYCMCKGTCCYTNGYSGRCRD |
| sequence | CKTCTYKCYVGKAMGNNNNNNCGCGCTGMGTRTTCCCGCCTTKTGACNNYA |
| 75%_Identity | RVYKRGCGARTCCKGGGAGRGGRYWGWTGACGTCAACAKTCVGGCTCCATG |
| | GCG (SEQ ID NO: 673) |
| Chiroptera | TGAGCTTCCCTCCGCCCTNBRVGDRRRDVVNNNBBBBDBNBGSVRRHTATA |
| Alignment | TRAGRNNCCYDYWYSKVWAGRCMTTTYWHRRKTASGGTGAYTTCCCACAAY |
| consensus | RCATAGCGACATGYAAATDHNNHNRGGYRTGCYTYCHCKGKCCYYNGYNRR |
| sequence | MRNCDYCTYKNYNNNNMGNNNNNNSGNNCTGHGHRTTCCCGCCTTBTGRCN |
| 85%_Identity | NYRRVYBRGCGARTNCDGGGARRRGRYWGDTKAYGTCRNNNNNNNNNACWK |
| | TYVSGCTCSATGGCG (SEQ ID NO: 674) |
| Chiroptera | TGAGCTTCNCTCCGCCCTNBRVRDRRRDNNNNNNBBBDBNBVVVRRHTATA |
| Alignment | TRAGRNNCCYDBHYSKVDRGDYMTTTHWHRRKKABGGTGAYTTCCCACAAY |
| consensus | RCAHAGCGACATGYAAATDHNNNNRGRYRTGYYTYCHCBGKCCYYNGYNRD |
| sequence | MNNYDYNNNKNNNNNNMNNNNNNNSNNNSYGNBHDWTCCCGCCTTBNGRNN |
| 90%_Identity | NYRNVBBRGCGARTNCDGGGARVRRRYDGDTKAYGTVRNNNNNNNNNRYWB |
| | WBVSGCWYSATGGCG (SEQ ID NO: 675) |
| Chiroptera | TGAGCTTCNCTCCGCCCTNBRVRDRRDNNNNNNNNNNNBNNVVVVRNTATA |
| Alignment | TRAGRNNCCHDNNHBKVDDRDHMTTTHNHRVDKABRGYRAYTTCCCAYAAY |
| consensus | RCMHRGCRAYATGYAAATDNNNNNRRDBDYGYYKBYNBNSNYYYBNNNNNN |
| sequence | HNNNNNNNNNNNNNNNNNNNNNNNNNNNSNNNBHDNTCCCGCCTYNNNNNN |
| 95%_Identity | NNNNVBNDRCRARTNCNRGGARVRRRNDGNTKAYGYVRNNNNNNNNNRYWB |
| | HBNBGCDYNATGGCG (SEQ ID NO: 676) |
| Chiroptera | TGAGCTTCNCKCCGCCCYNNRVVNVVNNNNNNNNNNNNNNNVNNVVNTWWA |
| Alignment | KVWRVNNNBYHNNNNBDNNNDNHMYYTHNNVVNKABDGYRAYNTTCCCAYR |
| consensus | RBRCHHVGCRAYAYGYAAAWDNNNNNNDDBDYSYBNBYNNNNNBNNBNNNN |
| sequence | NNNNNNNNNNNNNNNNNNNNNNNNNNNNNNNNNNNNNNNNNTYYYGBYHNN |
| 99%_Identity | NNNNNNNNNNNNNNDRNDRVKNYNRGGRRVRVNNNNNNGNTBWYGHNNVNN |
| | NNNNNNNVYDNNNNNNNNYNATGGCG (SEQ ID NO: 677 |
| Chiroptera | NVVNKABDGYRAYNTTCCCAYRRBRCHHVGCRAYAYGYAAAWDNNNNNNDD |
| Alignment | BDYSYBNBYNNNNNBNNBNNNNNNNNNNNNNNNNNNNNNNNNNNNNNNNNN |
| consensus | NNNNNNNNNNNNTYYYGBYHNNNNNNNNNNNNNNNNDRNDRVKNYNRGGRR |
| sequence | VRVNNNNNNGNTBWYGHNNVNNNNNNNNNVYDNNNNNNNNYNATGGCG |
| 100%_Identity | (SEQ ID NO: 678) |

In some embodiments, the promoter comprises a nucleotide sequence having at least 85%, 90%, 95%, 96%, 97%, 98%, 99%, or 100% identity to nucleotides 20-253 of any one of SEQ ID NOs: 673-678 or a functional fragment or variant (e.g., codon optimized) thereof.

### Dermoptera H1 Promoters

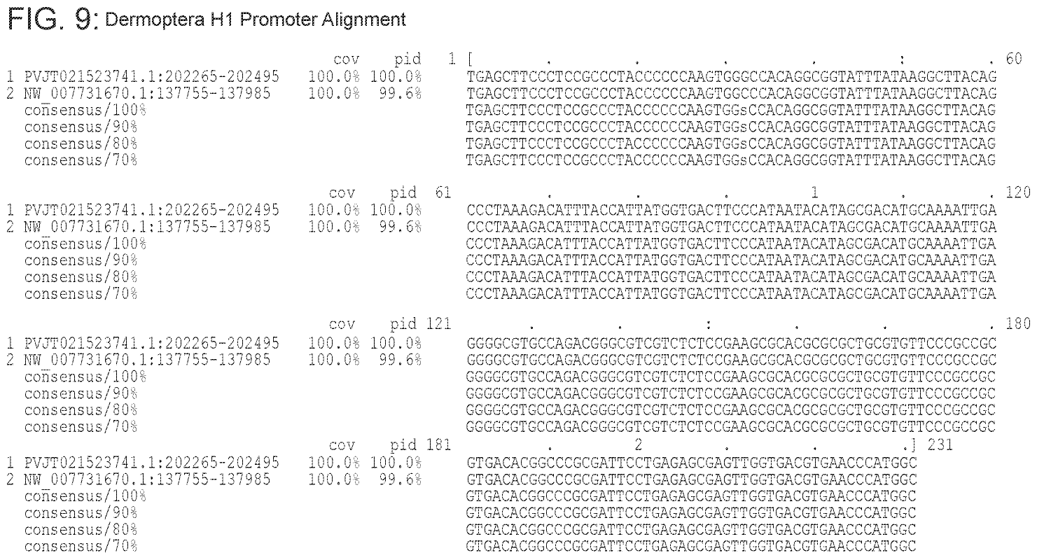

In certain embodiments, the promoter comprises a Dermoptera H1 promoter. An alignment of Dermoptera H1 promoter sequences is provided in FIG. 9 (wherein sequences numbered 1-2 in FIG. 9 correspond to SEQ ID NOs: 679 and 680, and consensus/100%, consensus/90%, consensus/80% and consensus/70% correspond to SEQ ID NOs: 1827-1830, respectively). In some embodiments, the promoter comprises a nucleotide sequence having at least 85%, 90%, 95%, 96%, 97%, 98%, 99%, or 100% identity to nucleotides 20-227 of any one of the sequences in FIG. 9 or a functional fragment or variant (e.g., codon optimized) thereof.

In certain embodiments, the Dermoptera H1 promoter comprises

TGAGCTTCCCTCCGCCCTACCCCCCAAGTGGSCCACAGGCGGTATTTATAAGGCTTACAGCC
CTAAAGACATTTACCATTATGGTGACTTCCCATAATACATAGCGACATGCAAAATTGAGGGG
CGTGCCAGACGGGCGTCGTCTCTCCGAAGCGCACGCGCGCTGCGTGTTCCCGCCGCGTGACA
CGGCCCGCGATTCCTGAGAGCGAGTTGGTGACGTGAACCCATGGC (SEQ ID NO: 681;
Dermoptera Alignment consensus sequence 100%_Identity)

In some embodiments, the promoter comprises a nucleotide sequence having at least 85%, 90%, 95%, 96%, 97%, 98%, 99%, or 100% identity to nucleotides 20-227 of SEQ ID NO: 681 or a functional fragment or variant (e.g., codon optimized) thereof.

### Hyracoidae H1 Promoters

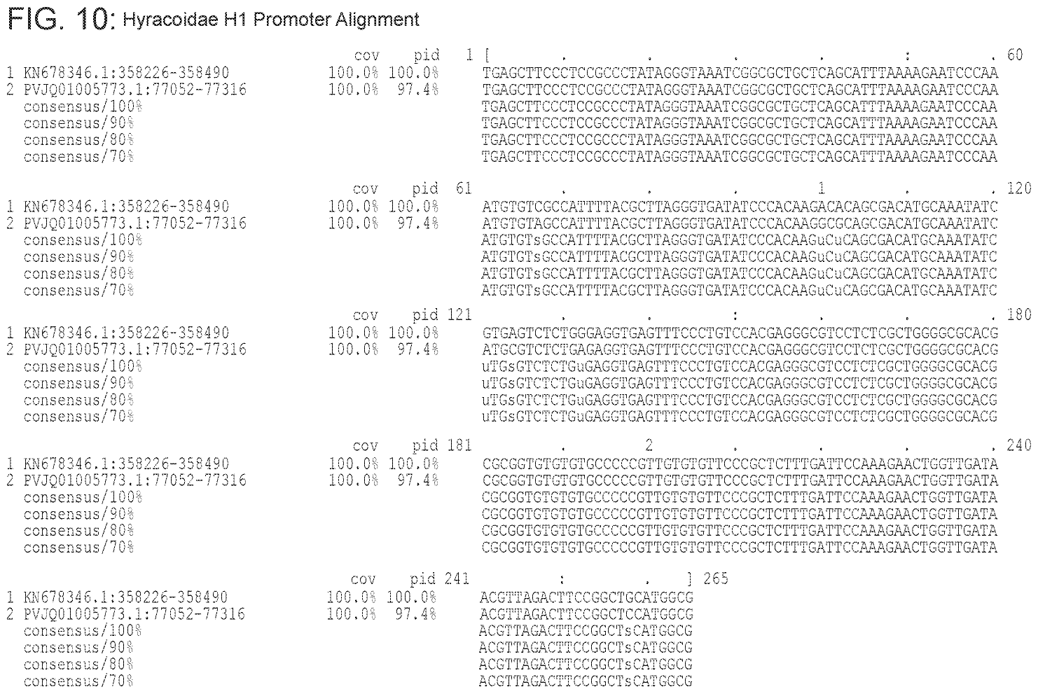

In certain embodiments, the promoter comprises an Hyracoidae H1 promoter. An alignment of Hyracoidae H1 promoter sequences is provided in FIG. 10 (wherein sequences numbered 1-2 in FIG. 10 correspond to SEQ ID NOs: 682 and 683, and consensus/100%, consensus/90%, consensus/80% and consensus/70% correspond to SEQ ID NOs: 1831-1834, respectively). In some embodiments, the promoter comprises a nucleotide sequence having at least 85%, 90%, 95%, 96%, 97%, 98%, 99%, or 100% identity to nucleotides 20-259 of any one of the sequences in FIG. 10 or a functional fragment or variant (e.g., codon optimized) thereof.

### Insectavora H1 Promoters

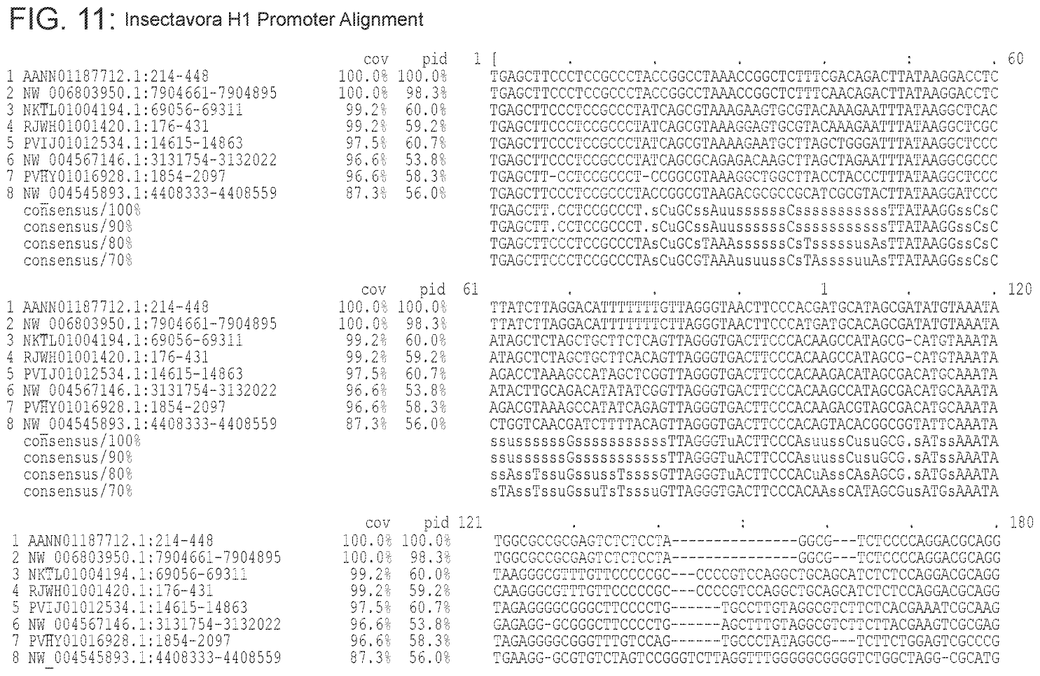

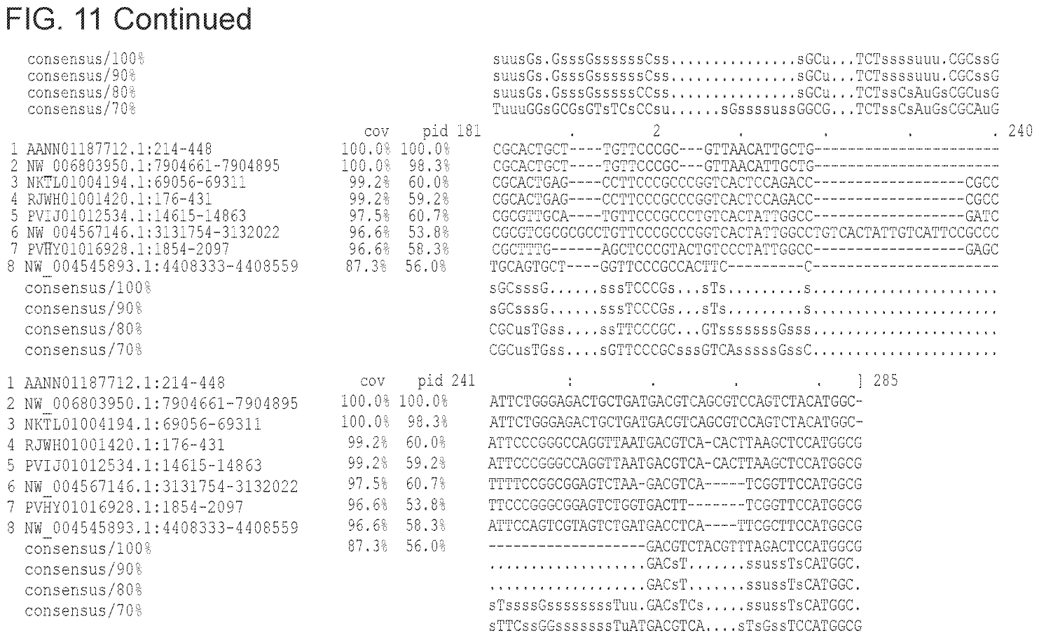

In certain embodiments, the promoter comprises an Insectavora H1 promoter. An alignment of Insectavora H1 promoter sequences is provided in FIG. 11 (wherein sequences numbered 1-8 in FIG. 11 correspond to SEQ ID NOs: 684-691, and consensus/100%, consensus/90%, consensus/80% and consensus/70% correspond to SEQ ID NOs: 1835-1838, respectively). In some embodiments, the promoter comprises a nucleotide sequence having at least 85%, 90%, 95%, 96%, 97%, 98%, 99%, or 100% identity to nucleotides 20-279 of any one of the sequences in FIG. 11 or a functional fragment or variant (e.g., codon optimized) thereof.

In certain embodiments, the Insectavora H1 promoter comprises a sequence selected from those in TABLE 9.

TABLE 9

| | |
|---|---|
| Insectavora | TGAGCTTCCCTCCGCCCTAYCRGCGTAAAVSRRBKCKTASMWMRRAYTTAT |
| Alignment | AAGGMYCYCWTASYTHWRGMYRTWTYWYDGTTAGGGTGACTTCCCACAAKM |
| consensus | CATAGCGAYATGYAAATATRRVGGSGCGKGTYTCYCCKVGGTCYYHGYYYW |
| sequence | GKMGGCGKCWTCTYHCSARGWCGCARGCGCRYTGMKCGCCYGTTCCCGCCC |
| 75%_Identity | KGTCAMYMYWGVYCTGTCACTATTGTCATTCCSRBCWTTCYSGGVSVMKKY |
| | TRATGACGTCARCRYYTMGKYTCCATGGCG (SEQ ID NO: 692) |
| Insectavora | TGAGCTTCCCTCCGCCCTAYCRGCSTAAAVVVNBKCKTWSMWMRNAYTTAT |
| Alignment | AAGGMYCNCWKABYTHWRGMYRYWTYWYDGTTAGGGTRACTTCCCACRAKV |
| consensus | CAYAGCGRYATGYAAATABRRVGSSGYKDGYYYVYCCNVGGTCYYHGBYYW |
| sequence | RKVKGCRKSDTCTYHCSARGWCGCVNGCGCRYTGMKCGCCNSTTCCCGCMM |
| 85%_Identity | BGTYAMYMYWGVYSTGTCACTATTGTCATTCCSVBCWTTCYSGGVSVMKKY |
| | TRATGACBTCARCRYYYMRNYTMCATGGCG (SEQ ID NO: 693) |
| Insectavora | TGAGCTTCCCTCCGCCCTAYCRGCSYARRVVVNNBCKYWBVDVVNMYTTAT |
| Alignment | AAGGMBCNCHKRBBYNHVGMYVYWKHWBDSTTAGGGTRACTTCCCAYRRKV |
| consensus | CRYRGCGRYATKYAAATABRRVGSSGYKDGYYYVBYCNVGGTCYYHGBYYW |
| sequence | RKVKGCRKSDTCTBNYBRRRWCGCVNGYGCDBYGMDCGCCNSYTCCCGYMM |
| 90%_Identity | BKTYMMYMYWGVYSTGTCACTATTGTCATTCCSVBCWTYYYVGKVSNMKKY |
| | TRRTGACBTCWRCRYYYMRNYTMCATGGCG (SEQ ID NO: 694) |
| Insectavora | TGAGCTTCCCTCCGCCCTAYCRGCSYARRVVVNNBCKYWBVDVVNMYTTAT |
| Alignment | AAGGMBCNCHKRBBYNHVGMYVYWKHWBDSTTAGGGTRACTTCCCAYRRKV |
| consensus | CRYRGCGRYATKYAAATABRRVGSSGYKDGYYYVBYCNVGGTCYYHGBYYW |
| sequence | RKVKGCRKSDTCTBNYBRRRWCGCVNGYGCDBYGMDCGCCNSYTCCCGYMM |
| 95%_Identity | BKTYMMYMYWGVYSTGTCACTATTGTCATTCCSVBCWTYYYVGKVSNMKKY |
| | TRRTGACBTCWRCRYYYMRNYTMCATGGCG (SEQ ID NO: 695) |
| Insectavora | TGAGCTTCCCTCCGCCCTAYCRGCSYARRVVVNNBCKYWBVDVVNMYTTAT |
| Alignment | AAGGMBCNCHKRBBYNHVGMYVYWKHWBDSTTAGGGTRACTTCCCAYRRKV |
| consensus | CRYRGCGRYATKYAAATABRRVGSSGYKDGYYYVBYCNVGGTCYYHGBYYW |
| sequence | RKVKGCRKSDTCTBNYBRRRWCGCVNGYGCDBYGMDCGCCNSYTCCCGYMM |
| 99%_Identity | BKTYMMYMYWGVYSTGTCACTATTGTCATTCCSVBCWTYYYVGKVSNMKKY |
| | TRRTGACBTCWRCRYYYMRNYTMCATGGCG (SEQ ID NO: 696) |
| Insectavora | TGAGCTTCCCTCCGCCCTAYCRGCSYARRVVVNNBCKYWBVDVVNMYTTAT |
| Alignment | AAGGMBCNCHKRBBYNHVGMYVYWKHWBDSTTAGGGTRACTTCCCAYRRKV |
| consensus | CRYRGCGRYATKYAAATABRRVGSSGYKDGYYYVBYCNVGGTCYYHGBYYW |
| sequence | RKVKGCRKSDTCTBNYBRRRWCGCVNGYGCDBYGMDCGCCNSYTCCCGYMM |
| 100%_Identity | BKTYMMYMYWGVYSTGTCACTATTGTCATTCCSVBCWTYYYVGKVSNMKKY |
| | TRRTGACBTCWRCRYYYMRNYTMCATGGCG (SEQ ID NO: 697) |

In some embodiments, the promoter comprises a nucleotide sequence having at least 85%, 90%, 95%, 96%, 97%, 98%, 99%, or 100% identity to nucleotides 20-278 of any one of SEQ ID NOs: 692-697 or a functional fragment or variant (e.g., codon optimized) thereof.

### Lagomorpha H1 Promoters

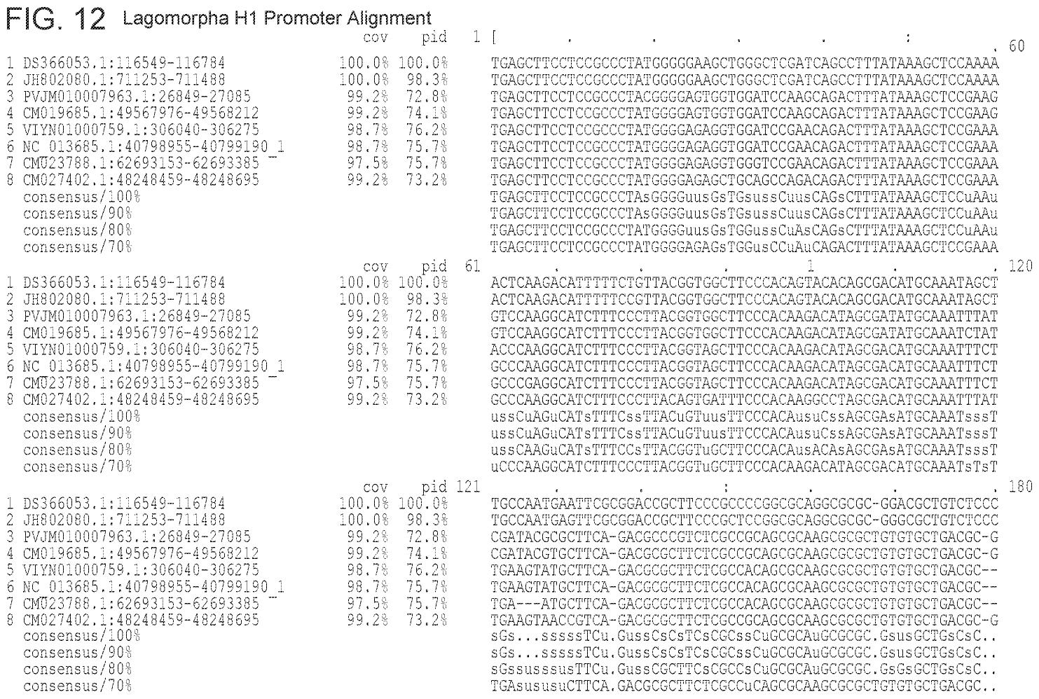

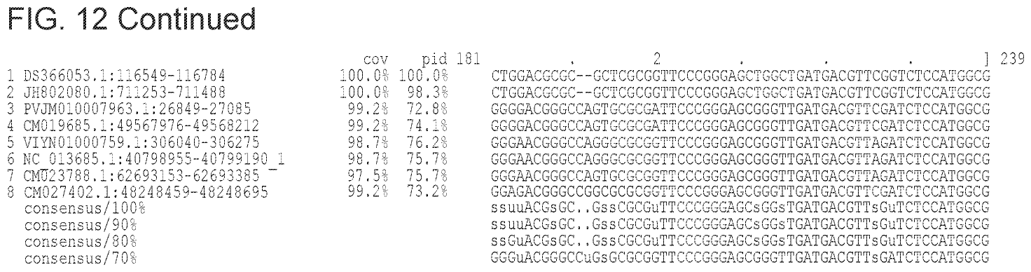

In certain embodiments, the promoter comprises a Lagomorpha H1 promoter. An alignment of Lagomorpha H1 promoter sequences is provided in FIG. 12 (wherein sequences numbered 1-8 in FIG. 12 correspond to SEQ ID NOs: 698-705, and consensus/100%, consensus/90%, consensus/80% and consensus/70% correspond to SEQ ID NOs: 1839-1842, respectively). In some embodiments, the promoter comprises a nucleotide sequence having at least 85%, 90%, 95%, 96%, 97%, 98%, 99%, or 100% identity to nucleotides 20-233 of any one of the sequences in FIG. 12 or a functional fragment or variant (e.g., codon optimized) thereof.

In certain embodiments, the Lagomorpha H1 promoter comprises a sequence selected from those in TABLE 10.

TABLE 10

| Lagomorpha | TGAGCTTCCTCCGCCCTATGGGGAGAGSTGGRYCCRADCAGACTTTATAAA |
|---|---|
| Alignment | GCTCCGAAARCCCAAGGCATCTTTCCCTTACGGTRGCTTCCCACAAGACAT |
| consensus | AGCGACATGCAAATWTMTTGAHRHDKRCTTCACGACGCGCTTCTCGCCRCA |
| sequence | GCGCAAGCGCGCTGTGTGCTGACGCCSGGGRACGGGCCAGYGCGCGGTTCC |
| 75%_Identity | CGGGAGCGGGTTGATGACGTTMGATCTCCATGGCG (SEQ ID NO: |
| | 706) |
| Lagomorpha | TGAGCTTCCTCFCGCCCTATGGGGRRWGSTGGRYYCRADCAGMCTTTATAA |
| Alignment | AGCTCCRAARRYYCAAGRCATYTTTCCSTTACGGTRGCTTCCCACARKACA |
| consensus | YAGCGAYATGCAAATWKMTYGMHRHDNRVTTCRCGRMSCGCTTCYCGCCVC |
| sequence | RGCGCARGCGCGCTGKGYGCTGWCKCCSSKGRACGSGCCRGBKCGCGRTTC |
| 85%_Identity | CCGGGAGCKGGYTGATGACGTTMGRTCTCCATGGCG (SEQ ID NO: |
| | 707) |
| Lagomorpha | TGAGCTTCCTCCGCCCTAYGGGGRRWGSTGSRBYCRRDCAGMCTTTATAAA |
| Alignment | GCTCCRAARRYYCRAGRCATYTTTCYSTTACRGTRRYTTCCCACARKRCMY |
| consensus | AGCGAYATGCAAATHKMTYGMHRHDNVVKTCRCGRMSCSCKTCYCGCYVCR |
| sequence | GCGCARGCGCGCTGKRYGCTGWCKCCSSKRRACGSGCCRGBKCGCGRTTCC |
| 90%_Identity | CGGGAGCKGGYTGATGACGTTMGRTCTCCATGGCG (SEQ ID NO: |
| | 708) |
| Lagomorpha | TGAGCTTCCTCCGCCCTAYGGGGRRWGSTGSRBYCRRDCAGMCTTTATAAA |
| Alignment | GCTCCRAARRYYCRAGRCATYTTTCYSTTACRGTRRYTTCCCACARKRCMY |
| consensus | AGCGAYATGCAAATHKMTYGMHRHDNVVKTCRCGRMSCSCKTCYCGCYVCR |
| sequence | GCGCARGCGCGCTGKRYGCTGWCKCCSSKRRACGSGCCRGBKCGCGRTTCC |
| 95%_Identity | CGGGAGCKGGYTGATGACGTTMGRTCTCCATGGCG (SEQ ID NO: |
| | 709) |
| Lagomorpha | TGAGCTTCCTCCGCCCTAYGGGGRRWGSTGSRBYCRRDCAGMCTTTATAAA |
| Alignment | GCTCCRAARRYYCRAGRCATYTTTCYSTTACRGTRRYTTCCCACARKRCMY |
| consensus | AGCGAYATGCAAATHKMTYGMHRHDNVVKTCRCGRMSCSCKTCYCGCYVCR |
| sequence | GCGCARGCGCGCTGKRYGCTGWCKCCSSKRRACGSGCCRGBKCGCGRTTCC |
| 99%_Identity | CGGGAGCKGGYTGATGACGTTMGRTCTCCATGGCG (SEQ ID NO: |
| | 710) |
| Lagomorpha | TGAGCTTCCTCCGCCCTAYGGGGRRWGSTGSRBYCRRDCAGMCTTTATAAA |
| Alignment | GCTCCRAARRYYCRAGRCATYTTTCYSTTACRGTRRYTTCCCACARKRCMY |
| consensus | AGCGAYATGCAAATHKMTYGMHRHDNVVKTCRCGRMSCSCKTCYCGCYVCR |
| sequence | GCGCARGCGCGCTGKRYGCTGWCKCCSSKRRACGSGCCRGBKCGCGRTTCC |
| 100%_Identity | CGGGAGCKGGYTGATGACGTTMGRTCTCCATGGCG (SEQ ID NO: |
| | 711) |

In some embodiments, the promoter comprises a nucleotide sequence having at least 85%, 90%, 95%, 96%, 97%, 98%, 99%, or 100% identity to nucleotides 20-233 of any one of SEQ ID NOs: 706-711 or a functional fragment or variant (e.g., codon optimized) thereof.

### Marsupial H1 Promoters

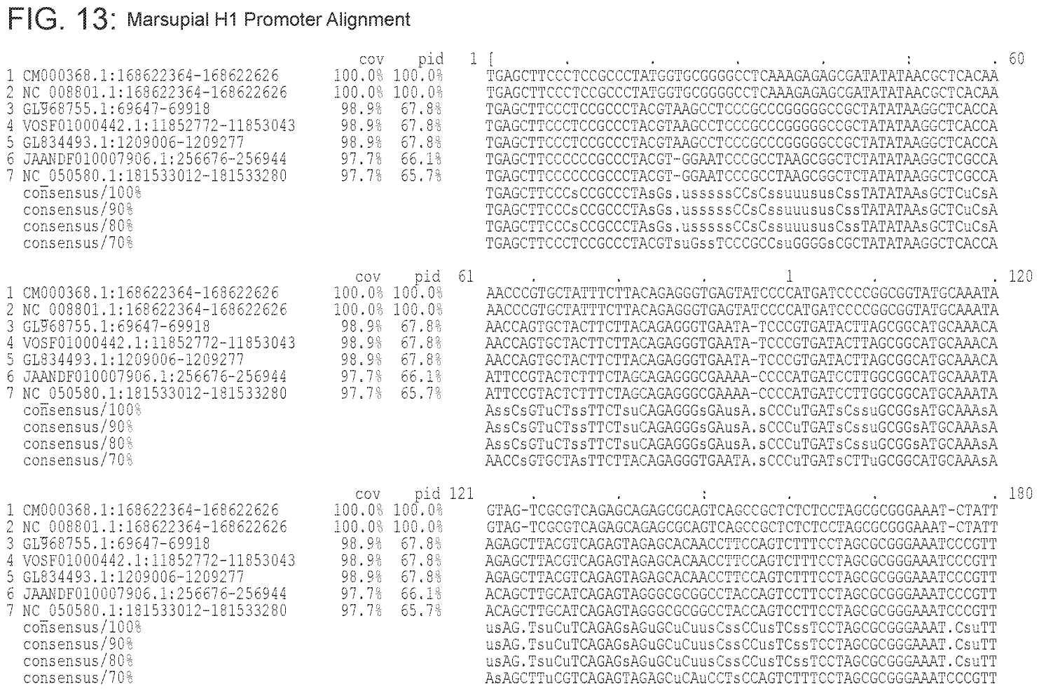

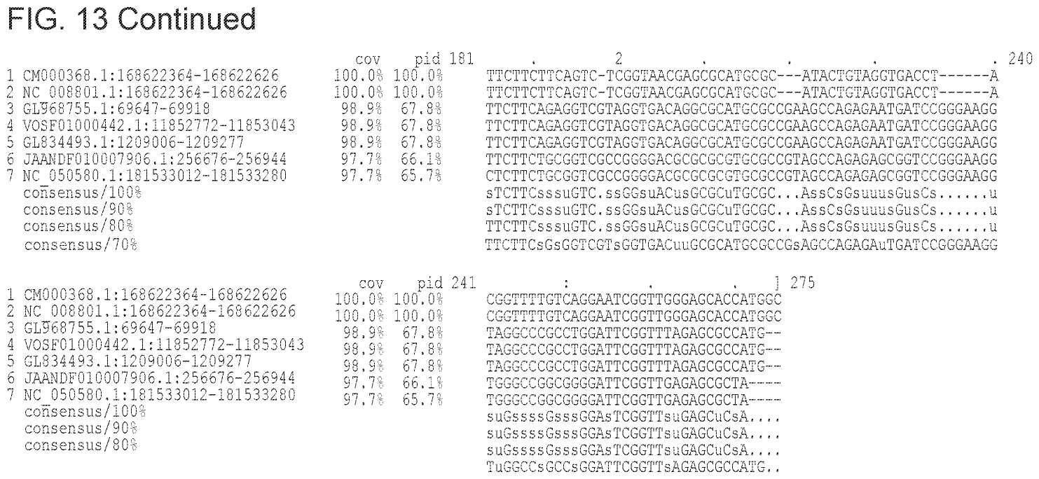

In certain embodiments, the promoter comprises a Marsupial H1 promoter. An alignment of Marsupial H1 promoter sequences is provided in FIG. 13 (wherein sequences numbered 1-7 in FIG. 13 correspond to SEQ ID NOs: 712-718, and consensus/100%, consensus/90%, consensus/80% and consensus/70% correspond to SEQ ID NOs: 1843-1846, respectively). In some embodiments, the promoter comprises a nucleotide sequence having at least 85%, 90%, 95%, 96%, 97%, 98%, 99%, or 100% identity to nucleotides 20-270 of any one of the sequences in FIG. 13 or a functional fragment or variant (e.g., codon optimized) thereof.

In certain embodiments, the Marsupial H1 promoter comprises a sequence selected from those in TABLE 11.

TABLE 11

| | |
|---|---|
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTTTCTTCWKMRGTCNYMGGKRACRV |
| 75%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 719) |
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTTTCTTCWKMRGTCNYMGGKRACRV |
| 85%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 720) |
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTYTCTTCWKMRGTCNYMGGKRACRV |
| 90%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 721) |
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTYTCTTCWKMRGTCNYMGGKRACRV |
| 95%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 722) |
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTYTCTTCWKMRGTCNYMGGKRACRV |
| 99%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 723) |
| Marsupial | TGAGCTTCCCYCCGCCCTAYGKNRSVVKSCCKCMHRRRSRSCKMTATATAA |
| Alignment | SGCTCRCMAAWYCMGTRCTMYTTCTWRCAGAGGGYGARWANYCCCRTGATM |
| consensus | CYYRGCGGYATGCAAAYARBAGNTYRCRTCAGAGYAGRGCRCRRYCWDCCR |
| sequence | STCYYTCCTAGCGCGGGAAATNCYRTTYTCTTCWKMRGTCNYMGGKRACRV |
| 100%_Identity | GCGCRTGCGCNNNAKMCWGWRRRYGRYCYNNNNNNRYRGKYYBGYSDGGAW |
| | TCGGTTKRGAGCRCYATGGC (SEQ ID NO: 724) |

In some embodiments, the promoter comprises a nucleotide sequence having at least 85%, 90%, 95%, 96%, 97%, 98%, 99%, or 100% identity to nucleotides 20-270 ofany one of SEQ ID NOs: 719-724 or a functional fragment or variant (e.g., codon optimized) thereof.

### Pangolin H1 Promoters

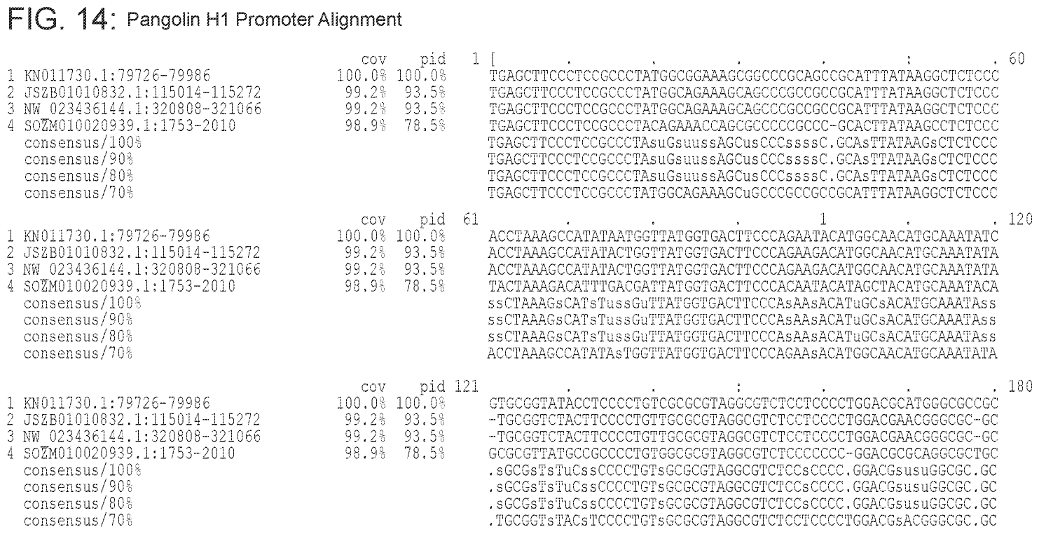

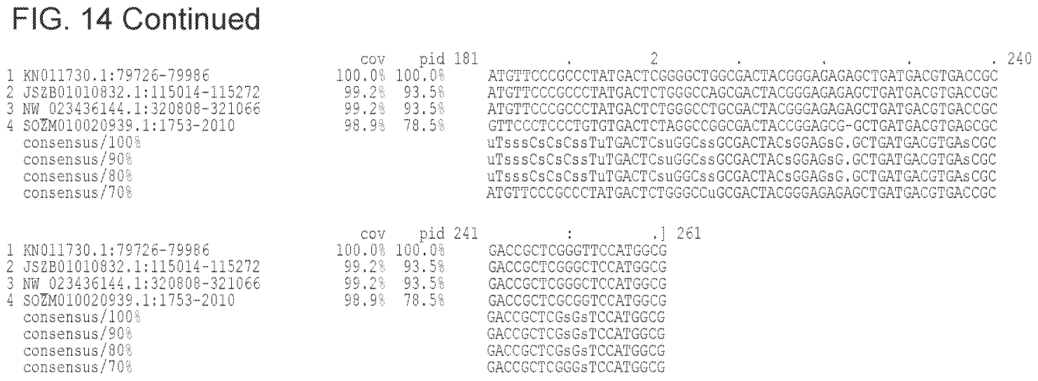

In certain embodiments, the promoter comprises an Pangolin H1 promoter. An alignment of Pangolin H1 promoter sequences is provided in FIG. 14 (wherein sequences numbered 1-4 in FIG. 14 correspond to SEQ ID NOs: 725-728, and consensus/100%, consensus/90%, consensus/80% and consensus/70% correspond to SEQ ID NOs: 1847-1850, respectively). In some embodiments, the promoter comprises a nucleotide sequence having at least 85%, 90%, 95%, 96%, 97%, 98%, 99%, or 100% identity to nucleotides 20-255 of any one of the sequences in FIG. 14 or a functional fragment or variant (e.g., codon optimized) thereof.

In certain embodiments, the Pangolin H1 promoter comprises a sequence selected from those in TABLE 12.

TABLE 12

| | |
|---|---|
| Pangolin | TGAGCTTCCCTCCGCCCTATGGCAGAAAGCRGCCCGCCGCCGCATTTATAA |
| Alignment | GGCTCTCCCACCTAAAGCCATATAMTGGTTATGGTGACTTCCCAGAAKACA |
| consensus | TGGCAACATGCAAATATANTGCGGTMTACYTCCCCTGTBGCGCGTAGGCGT |
| sequence | CTCCTCCCCTGGACGMACGGGCGCNGCATGTTCCCGCCCTATGACTCTGGG |
| 75%_Identity | CCDGCGACTACGGGAGAGAGCTGATGACGTGACCGCGACCGCTCGGGBTCC |
| | ATGGCG (SEQ ID NO: 729) |
| Pangolin | TGAGCTTCCCTCCGCCCTAYRGMRRMMAGCRSCCCSSMSCNGCAYTTATAA |
| Alignment | GSCTCTCCCWMCTAAAGMCATWTRMYGRTTATGGTGACTTCCCASAAKACA |
| consensus | TRGCWACATGCAAATAYMNYGCGKTMTRCYKCCCCTGTBGCGCGTAGGCGT |
| sequence | CTCCYCCCCNGGACGMRYRGGCGCNGCRTKYYCYCSCYSTRTGACTCKRGG |
| 85%_Identity | CYDGCGACTACSGGAGMGNGCTGATGACGTGASCGCGACCGCTCGSGBTCC |
| | ATGGCG (SEQ ID NO: 730) |
| Pangolin | TGAGCTTCCCTCCGCCCTAYRGMRRMMAGCRSCCCSSMSCNGCAYTTATAA |
| Alignment | GSCTCTCCCWMCTAAAGMCATWTRMYGRTTATGGTGACTTCCCASAAKACA |
| consensus | TRGCWACATGCAAATAYMNYGCGKTMTRCYKCCCCTGTBGCGCGTAGGCGT |
| sequence | CTCCYCCCCNGGACGMRYRGGCGCNGCRTKYYCYCSCYSTRTGACTCKRGG |
| 90%_Identity | CYDGCGACTACSGGAGMGNGCTGATGACGTGASCGCGACCGCTCGSGBTCC |
| | ATGGCG (SEQ ID NO: 731) |
| Pangolin | TGAGCTTCCCTCCGCCCTAYRGMRRMMAGCRSCCCSSMSCNGCAYTTATAA |
| Alignment | GSCTCTCCCWMCTAAAGMCATWTRMYGRTTATGGTGACTTCCCASAAKACA |
| consensus | TRGCWACATGCAAATAYMNYGCGKTMTRCYKCCCCTGTBGCGCGTAGGCGT |
| sequence | CTCCYCCCCNGGACGMRYRGGCGCNGCRTKYYCYCSCYSTRTGACTCKRGG |
| 95%_Identity | CYDGCGACTACSGGAGMGNGCTGATGACGTGASCGCGACCGCTCGSGBTCC |
| | ATGGCG (SEQ ID NO: 732) |
| Pangolin | TGAGCTTCCCTCCGCCCTAYRGMRRMMAGCRSCCCSSMSCNGCAYTTATAA |
| Alignment | GSCTCTCCCWMCTAAAGMCATWTRMYGRTTATGGTGACTTCCCASAAKACA |
| consensus | TRGCWACATGCAAATAYMNYGCGKTMTRCYKCCCCTGTBGCGCGTAGGCGT |
| sequence | CTCCYCCCCNGGACGMRYRGGCGCNGCRTKYYCYCSCYSTRTGACTCKRGG |
| 99%_Identity | CYDGCGACTACSGGAGMGNGCTGATGACGTGASCGCGACCGCTCGSGBTCC |
| | ATGGCG (SEQ ID NO: 733) |
| Pangolin | TGAGCTTCCCTCCGCCCTAYRGMRRMMAGCRSCCCSSMSCNGCAYTTATAA |
| Alignment | GSCTCTCCCWMCTAAAGMCATWTRMYGRTTATGGTGACTTCCCASAAKACA |
| consensus | TRGCWACATGCAAATAYMNYGCGKTMTRCYKCCCCTGTBGCGCGTAGGCGT |
| sequence | CTCCYCCCCNGGACGMRYRGGCGCNGCRTKYYCYCSCYSTRTGACTCKRGG |
| 100%_Identity | CYDGCGACTACSGGAGMGNGCTGATGACGTGASCGCGACCGCTCGSGBTCC |
| | ATGGCG (SEQ ID NO: 734) |

In some embodiments, the promoter comprises a nucleotide sequence having at least 85%, 90%, 95%, 96%, 97%, 98%, 99%, or 100% identity to nucleotides 20-255 of any one of SEQ ID NOs: 729-734 or a functional fragment or variant (e.g., codon optimized) thereof.

### Perissodactyla H1 Promoters

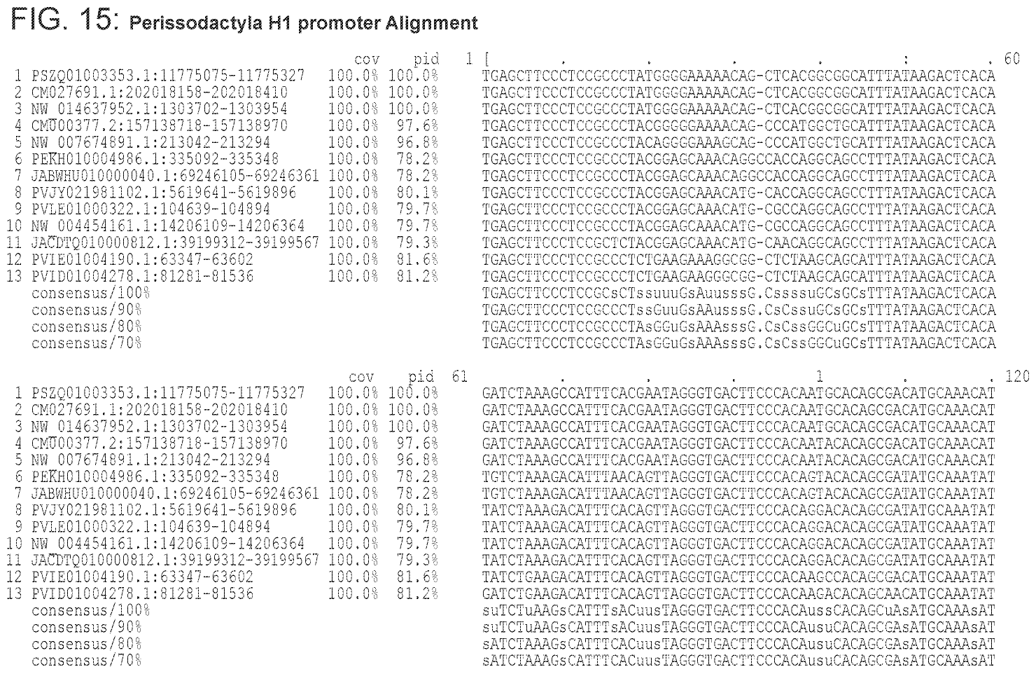

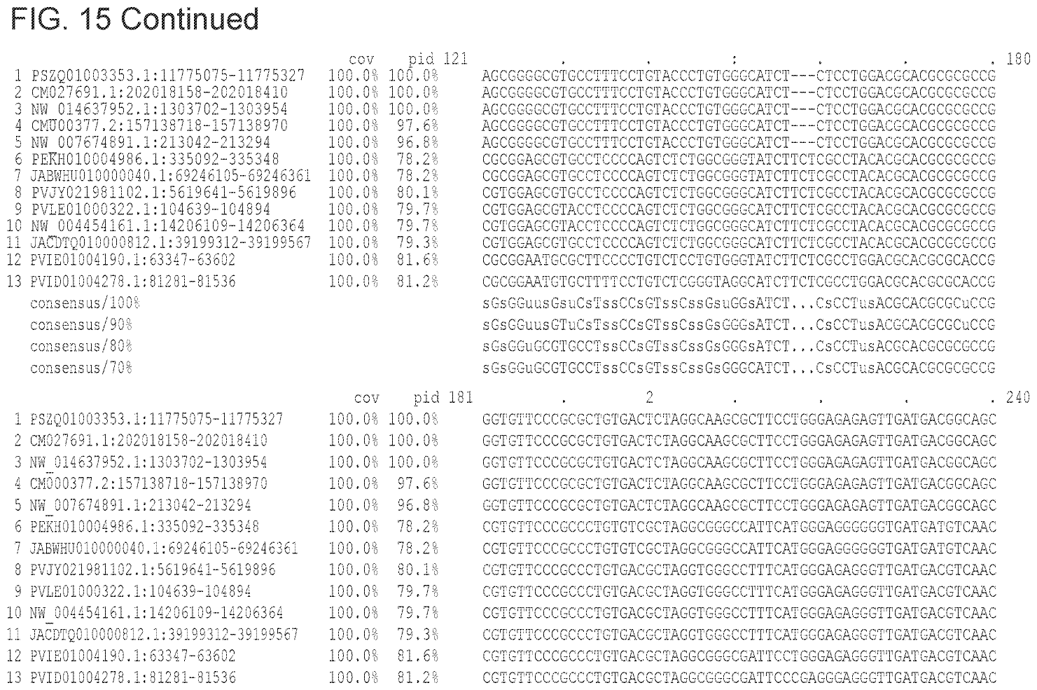

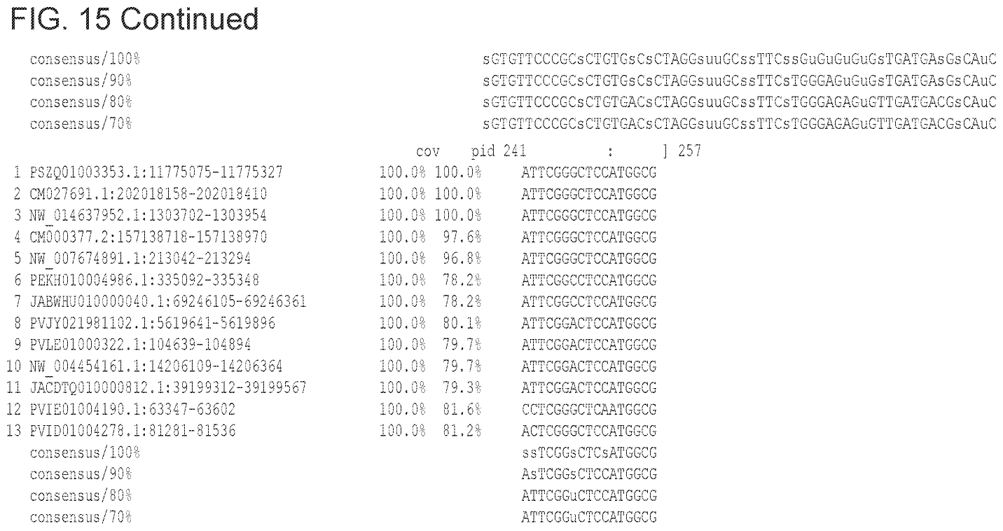

In certain embodiments, the promoter comprises an Perissodactyla H1 promoter. An alignment of Perissodactyla H1 promoter sequences is provided in FIG. 15 (wherein sequences numbered 1-13 in FIG. 15 correspond to SEQ ID NOs: 735-747, and consensus/100%, consensus/90%, consensus/80% and consensus/70% correspond to SEQ ID NOs: 1851-1854, respectively). In some embodiments, the promoter comprises a nucleotide sequence having at least 85%, 90%, 95%, 96%, 97%, 98%, 99%, or 100% identity to nucleotides 20-251 of any one of the sequences in FIG. 15 or a functional fragment or variant (e.g., codon optimized) thereof.

In certain embodiments, the Perissodactyla H1 promoter comprises a sequence selected from those in TABLE 13.

TABLE 13

| | |
|---|---|
| Perissodactyla | TGAGCTTCCCTCCGCCCTAYGGRGMAAAMMDGCNCMMGGCRGCMTTTATAA |
| Alignment | GACTCACAKATCTAAAGMCATTTCACRRWTAGGGTGACTTCCCACARKRCA |
| consensus | CAGCGAYATGCAAAYATMGYGGRGCGTGCCTYYCCWGTMYCYKGYGGGCAT |
| sequence | CTNNNCKCCTRSACGCACGCGCGCCGSGTGTTCCCGCSCTGTGACKCTAGG |
| 75%_Identity | YRRGCSHTTCMTGGGAGAGRGTTGATGACGKCARCATTCGGRCTCCATGGC |
| | G (SEQ ID NO: 748) |
| Perissodactyla | TGAGCTTCCCTCCGCCCTAYGGRGMAAAVMDGCNCMMGGCRGCMTTTATAA |
| Alignment | GACTCACAKATCTAAAGMCATTTCACRRWTAGGGTGACTTCCCACARKRCA |
| consensus | CAGCGAYATGCAAAYATMGYGGRGCGTGCCTYYCCWGTMYCYKGYGGGYAT |
| sequence | CTNNNCKCCTRSACGCACGCGCGCCGSGTGTTCCCGCSCTGTGACKCTAGG |
| 85%_Identity | YRRGCSHTTCMTGGGAGAGRGTTGATGACGKCARCATTCGGRCTCCATGGC |
| | G (SEQ ID NO: 749) |
| Perissodactyla | TGAGCTTCCCTCCGCCCTMYGRRGVAARVMDGNCNCHHRGCDGCMTTTATA |
| Alignment | AGACTCACAKRTCTRAAGMCATTTMACRRWTAGGGTGACTTCCCACARKRC |
| consensus | ACAGCGAYATGCAAAYATMGYGGRRYGTRCYTYYCCWGTMYCYKGYGGGYA |
| sequence | TCTNNNCKCCTRSACGCACGCGCRCCGSGTGTTCCCGCSCTGTGWCKCTAG |
| 90%_Identity | GYRRGCSHTTCMTGGGAGRGRGKTGATGAYGKCARCAYTCGGVCTCCATGG |
| | CG (SEQ ID NO: 750) |
| Perissodactyla | TGAGCTTCCCTCCGCYCTMYRRRGVARRVMDGNCNMHHRGCDGCMTTTATA |
| Alignment | AGACTCACAKRTCTRAAGMCATTTMACRRWTAGGGTGACTTCCCACARKVC |
| consensus | ACAGCRAYATGCAAAYATMGYGGRRYGYRCYTYYCCWGTMYCBKGYRGGYA |
| sequence | TCTNNNCKCCTRSACGCACGCGCRCCGSGTGTTCCCGCSCTGTGWCKCTAG |
| 95%_Identity | GYRRGCSHTTCMYGRGRGRGRGKTGATGAYGKCARCMYTCGGVCTCMATGG |
| | CG (SEQ ID NO: 751) |
| Perissodactyla | TGAGCTTCCCTCCGCYCTMYRRRGVARRVMDGNCNMHHRGCDGCMTTTATA |
| Alignment | AGACTCACAKRTCTRAAGMCATTTMACRRWTAGGGTGACTTCCCACARKVC |
| consensus | ACAGCRAYATGCAAAYATMGYGGRRYGYRCYTYYCCWGTMYCBKGYRGGYA |
| sequence | TCTNNNCKCCTRSACGCACGCGCRCCGSGTGTTCCCGCSCTGTGWCKCTAG |
| 99%_Identity | GYRRGCSHTTCMYGRGRGRGRGKTGATGAYGKCARCMYTCGGVCTCMATGG |
| | CG (SEQ ID NO: 752) |
| Perissodactyla | TGAGCTTCCCTCCGCYCTMYRRRGVARRVMDGNCNMHHRGCDGCMTTTATA |
| Alignment | AGACTCACAKRTCTRAAGMCATTTMACRRWTAGGGTGACTTCCCACARKVC |
| consensus | ACAGCRAYATGCAAAYATMGYGGRRYGYRCYTYYCCWGTMYCBKGYRGGYA |
| sequence | TCTNNNCKCCTRSACGCACGCGCRCCGSGTGTTCCCGCSCTGTGWCKCTAG |
| 100%_Identity | GYRRGCSHTTCMYGRGRGRGRGKTGATGAYGKCARCMYTCGGVCTCMATGG |
| | CG (SEQ ID NO: 753) |

In some embodiments, the promoter comprises a nucleotide sequence having at least 85%, 90%, 95%, 96%, 97%, 98%, 99%, or 100% identity to nucleotides 20-250 of any one of SEQ ID NOs: 748-753 or a functional fragment or variant (e.g., codon optimized) thereof.

### Primate H1 Promoters

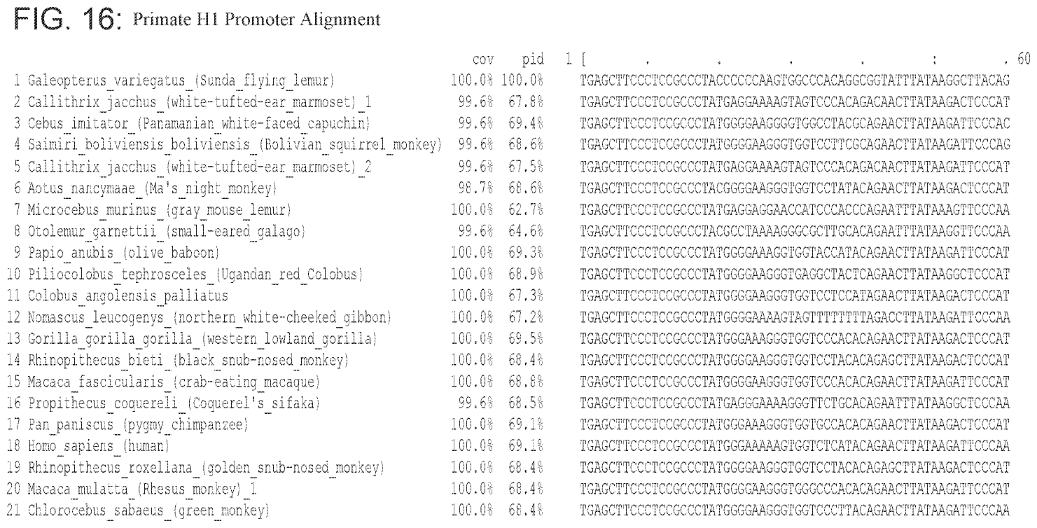

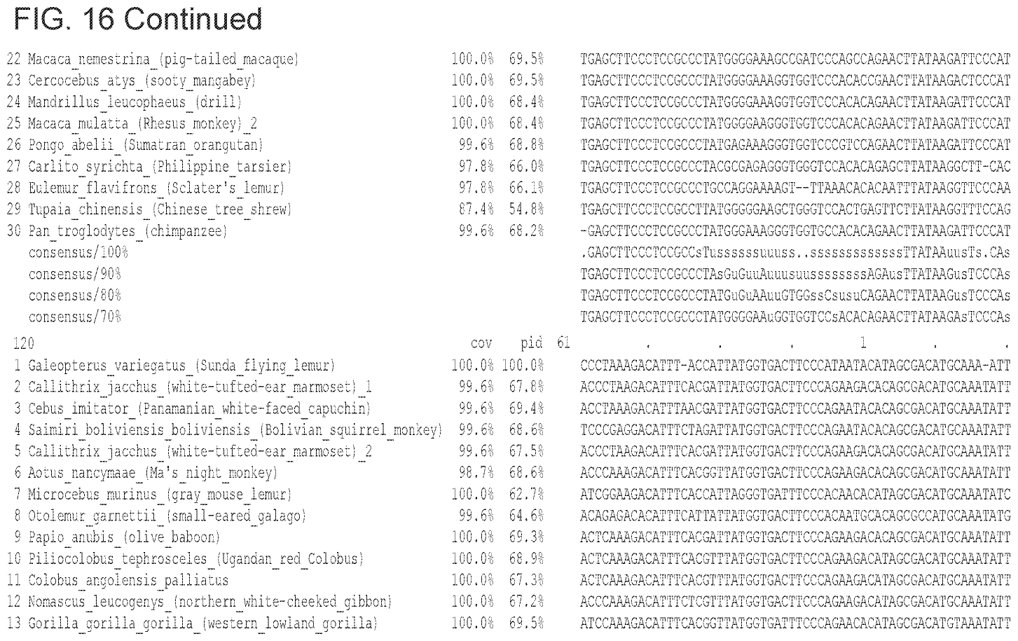

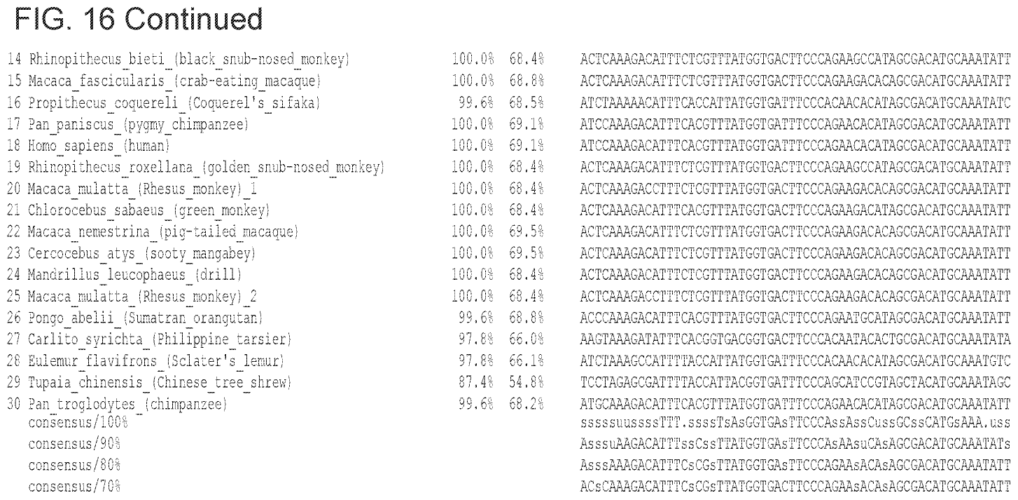

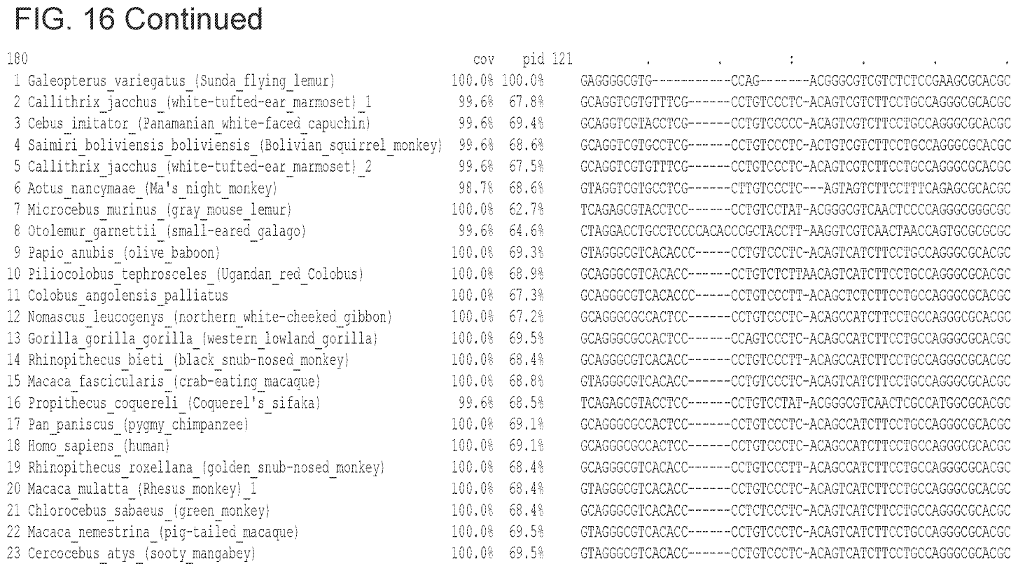

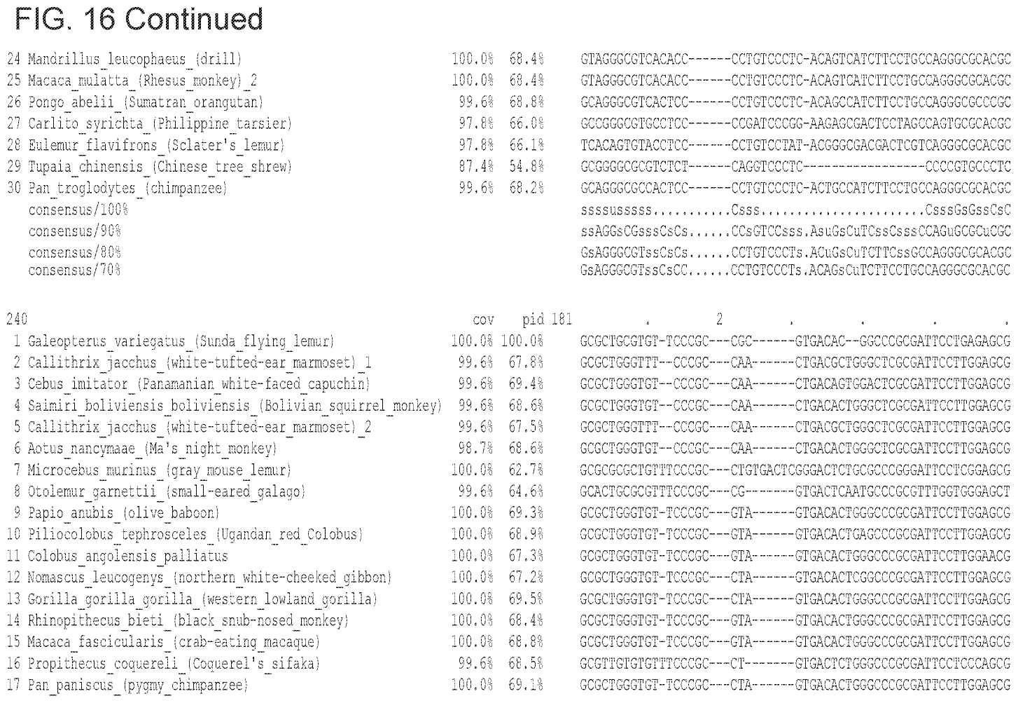

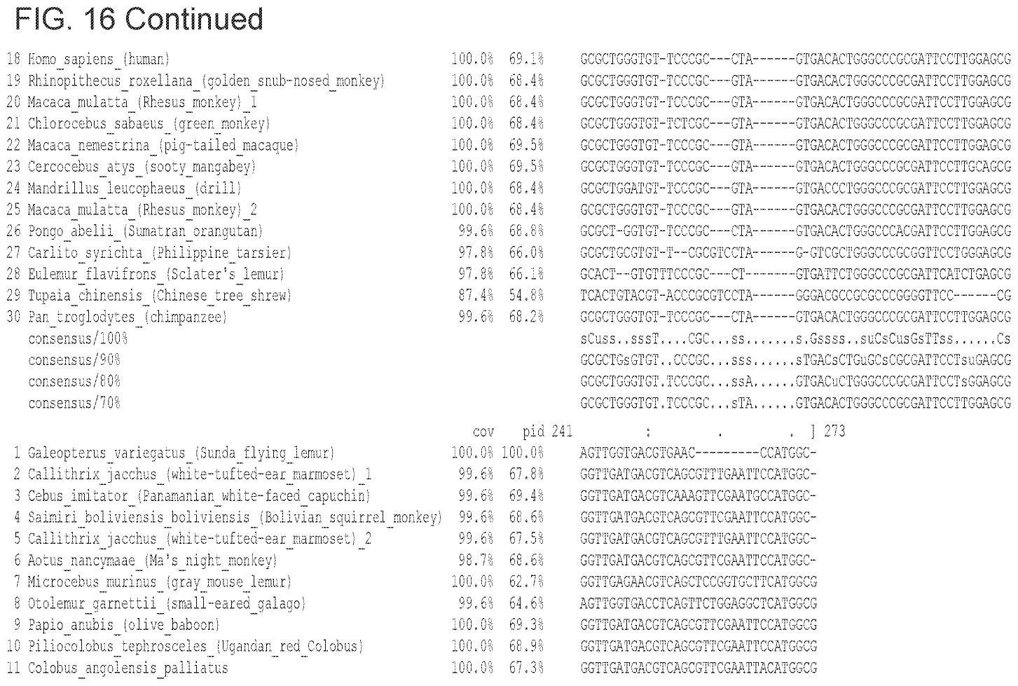

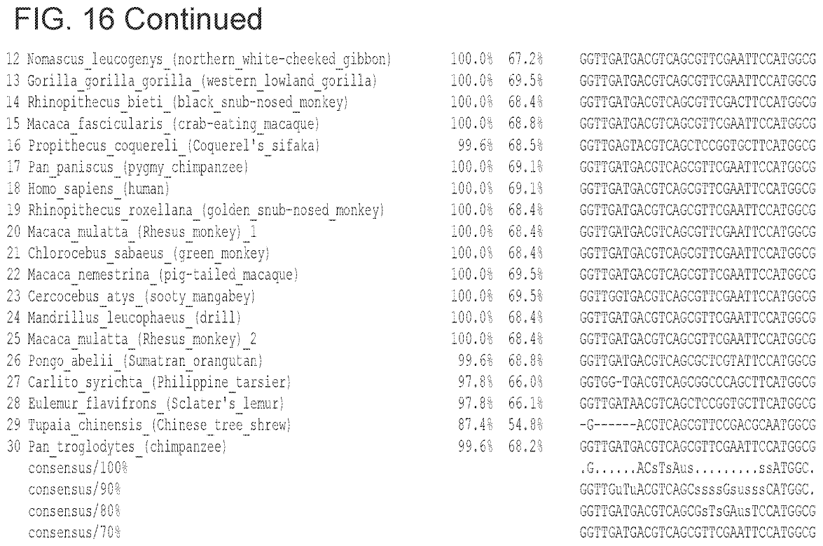

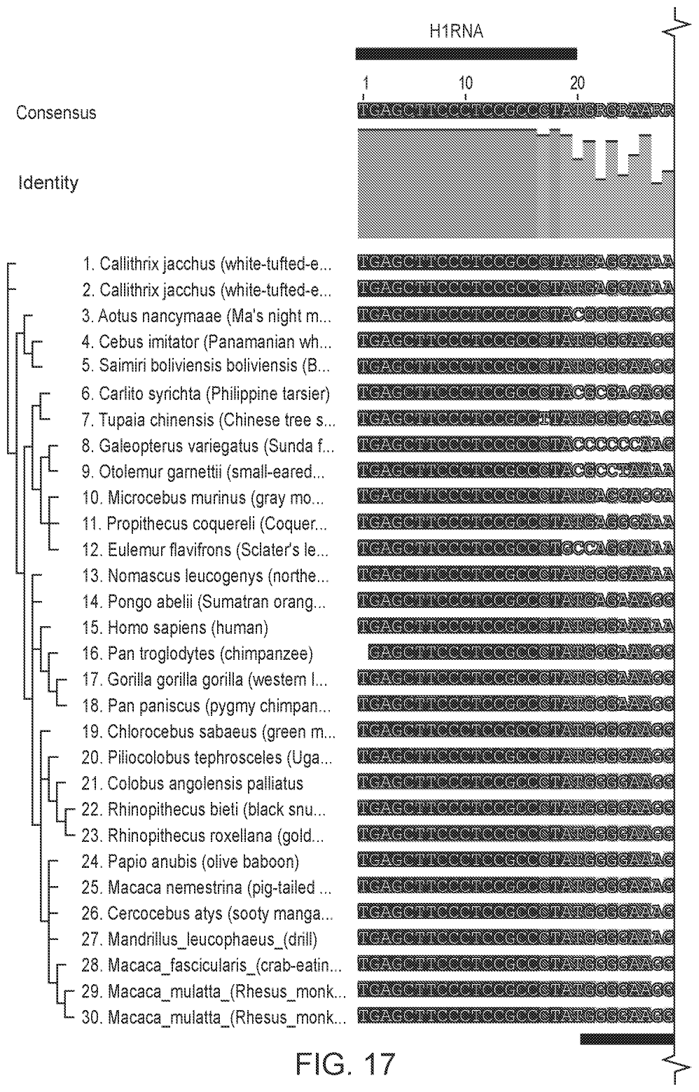

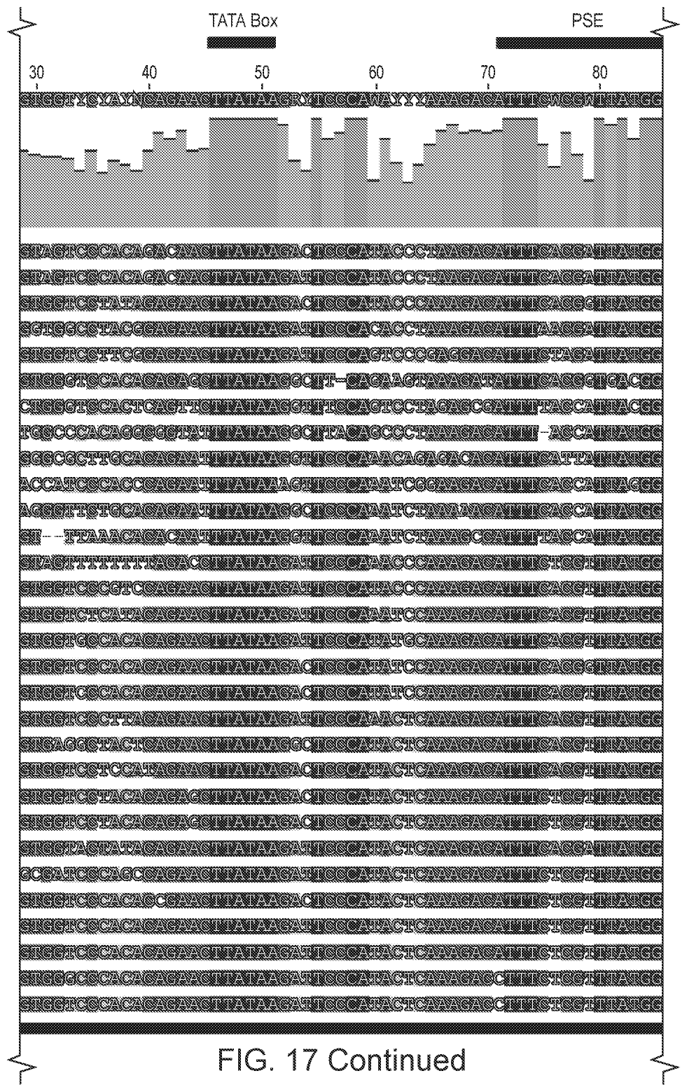

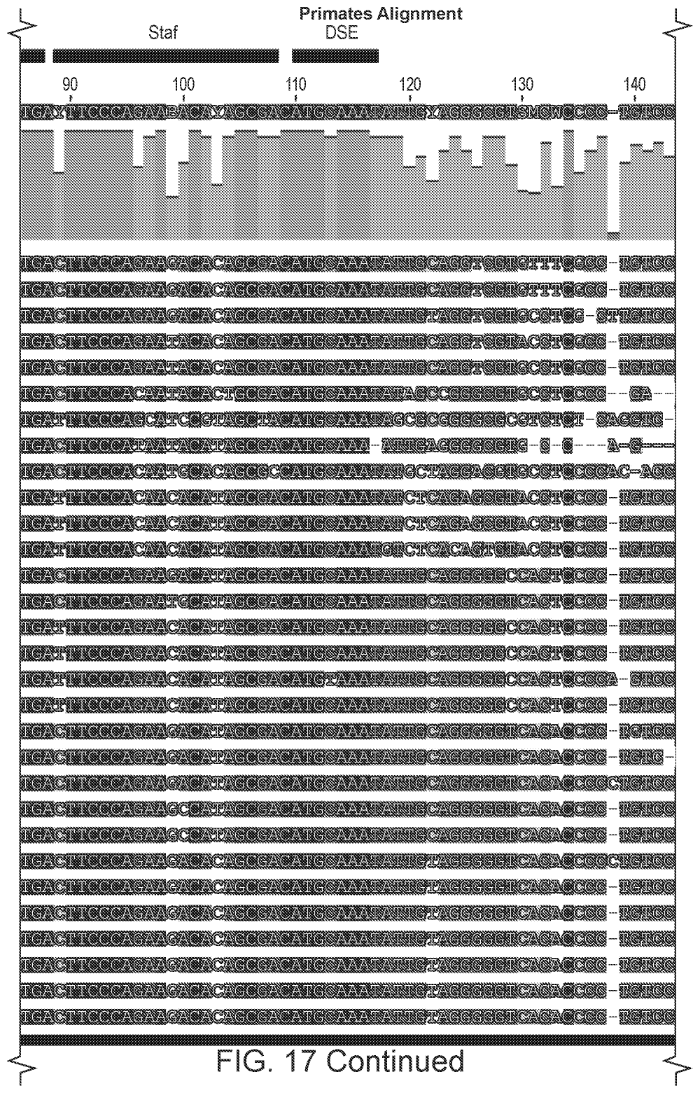

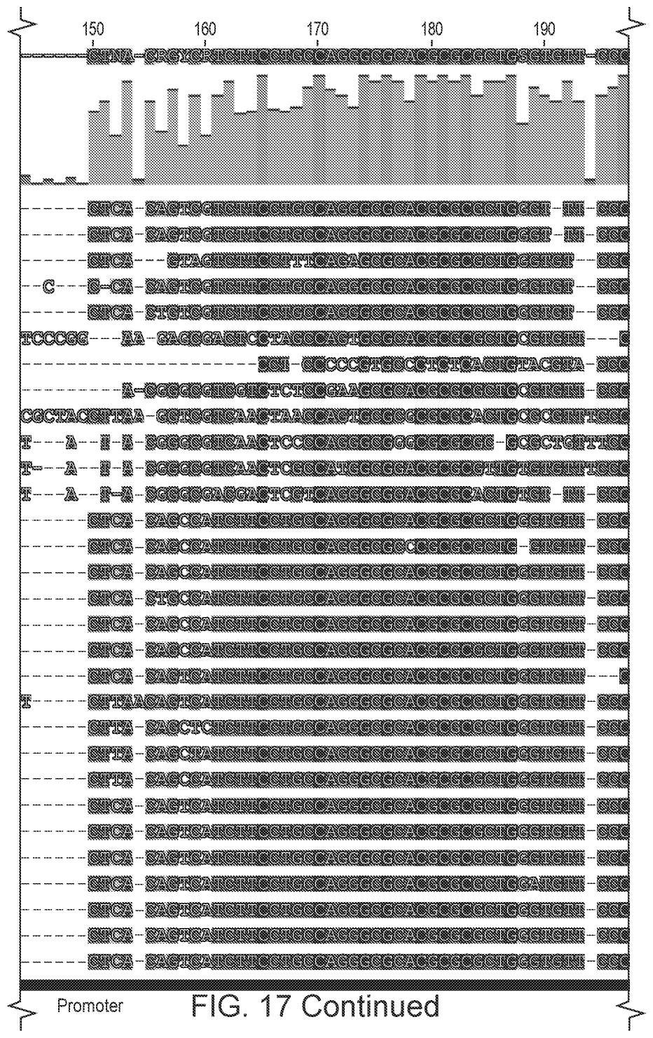

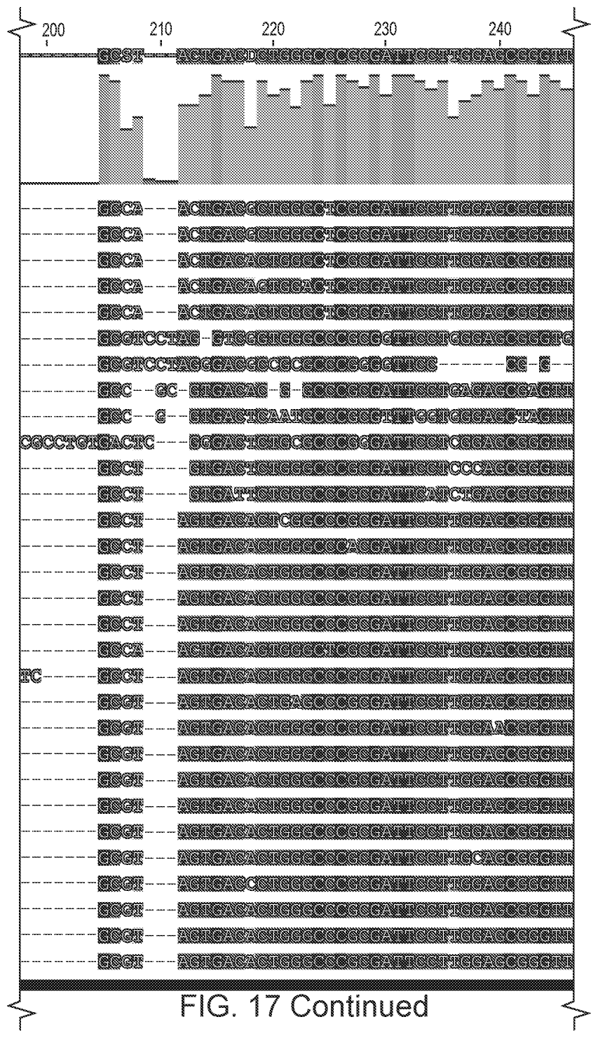

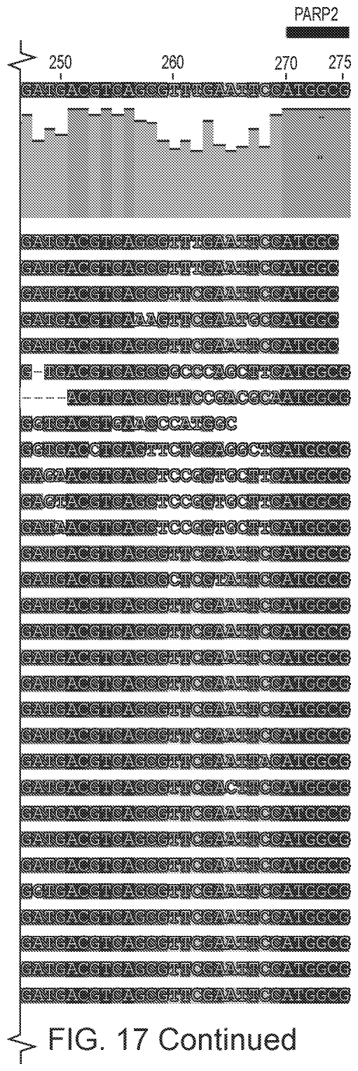

In certain embodiments, the promoter comprises a Primate H1 promoter. An alignment of Primate H1 promoter sequences is provided in FIG. 16 (wherein sequences numbered 1-30 in FIG. 16 correspond to SEQ ID NOs: 754-783, and consensus/100%, consensus/90%, consensus/80% and consensus/70% correspond to SEQ ID NOs: 1855-1858, respectively). FIG. 17 provides an second alignment of H1 promoter sequences from Primate species showing the TATA box, PSE, Staf, and DSE binding sites. Sequences numbered 1-30 in the alignment correspond to SEQ ID NOs: 755, 758, 759, 756, 757, 780, 783, 754, 761, 760, 769, 781, 765, 779, 771, 783, 766, 770, 774, 763, 764, 767, 772, 762, 775, 776, 777, 768, 773, and 788, respectively. The consensus sequence shown in FIG. 17 corresponds to SEQ ID NO: 1868. In some embodiments, the promoter comprises a nucleotide sequence having at least 85%, 90%, 95%, 96%, 97%, 98%, 99%, or 100% identity to nucleotides 20-267 of any one of the sequences in FIG. 16 or FIG. 17 or a functional fragment or variant (e.g., codon optimized) thereof. In certain embodiments, a functional fragment of a primate H1 promoter comprises at least a TATA box, or a PSE, Staf, or DSE binding site.

In certain embodiments, the Primate H1 promoter comprises a sequence selected from those in TABLE 14.

TABLE 14

| | |
|---|---|
| Primate | TGAGCTTCCCTCCGCCCTATGRGRAARRGTGGTYCYAYNCAGAACTTATAA |
| Alignment | GRYTCCCAWAYYYAAAGACATTTCWCGWTTATGGTGAYTTCCCAGAABACA |
| consensus | YAGCGACATGCAAATATTGYAGGGCGTSMCWCCCCTGTCCCTYACRGYCRT |
| sequence | CTTCCTGCCAGGGCGCACGCGCGCTGGGTGTTCCCGCSTAGTGACDCTGGG |
| 75%_Identity | CCCGCGATTCCTTGGAGCGGGTTGATGACGTCAGCGTTCGAATTCCATGGC |
| | G (SEQ ID NO: 784) |
| Primate | TGAGCTTCCCTCCGCCCTAYGRGRAARRVKRRKYYYDYNSAGARYTTATAA |
| Alignment | GRYTCCCADAYYYAAAGACATTTCWCSWTTATGGTGAYTTCCCASAABMCA |
| consensus | YAGCGACATGCAAATATYGYAGGKCGYSMCWCSCCKGTCCCWYACRGBCRT |
| sequence | CWWCYYKCCAGDGCGCACGCGCGCTGSGTGTNCCCGCSWNSTGACDCTGGG |
| 85%_Identity | CYCGCGATTCCTBGGAGCGGGTTGRTGACGTCAGCKYYSGWRYTYCATGGC |
| | G (SEQ ID NO: 785) |
| Primate | TGAGCTTCCCTCCGCCCTAYGRGRRARRVKRRKBYYDYNSAGARYTTATAA |
| Alignment | GRYTCCCADAYYYDAAGACATTTYWCSWTTATGGTGAYTTCCCASAABMCA |
| consensus | YAGCGACATGCAAATATYKYAGGKCGYVHCWCSCCKGTCCYWYANRGBCRT |
| sequence | CWWCYYKCCAGDGCGCVCGCGCGCTGSGTGTNNCCCGCSWNSTGACDCTGS |
| 90%_Identity | GCYCGCGATTCCTBNGAGCGGGTTGRTRACGTCAGCKYYSGWRYKYCATGG |
| | CG (SEQ ID NO: 786) |
| Primate | TGAGCTTCCCTCCGCCCTAYSVSNRARRVBNVKBHYDBNBVSWNYTTATAA |
| Alignment | GRYTYNCANWYBBDRAVMBMTTTNWHSDTTAYGGTGAYTTCCCASAABVCA |
| consensus | YAGCGACATGCAAATATNKYRGRKCGYVHYWCNNCHDSTNNYNNNNDNBNN |
| sequence | WCDNCYHNYCVNDGCGCVCGCGCRCTNBRYKTNNCNCGCNNNSDNSKGACD |
| 95%_Identity | CNNNGCYCGSGRTTCVTBNSANCGRGTNGNKNACGTCARHKNYBSNNNNYC |
| | ATGGCG (SEQ ID NO: 787) |
| Primate | TGAGCTTCCCTCCGCCYTRYSVSNVRRRNBNNBNHHNBNBVSWNYTTATAA |
| Alignment | RRYTYNCANHHNBDRRVMBMTTTNWHBDTKABGGTGAYTTCCCABMABVCR |
| consensus | YWGCKMCATGYAAANRKNBHVSRDYSYVNNNNNNNNNNNCHDVNNNNNNNN |
| sequence | NNNNNNNNNNNNNNNCVNNGYGSVCKCKCRYKNNVYKTNNNNCGCNNNSDN |
| 99%_Identity | NNNNNNSNGWYNSNNNRCYCRSGDTTSVNNNNNNCKNGNNNNNNACSTSAR |
| | HNNNNNNNNNHMATGGCG (SEQ ID NO: 788) |
| Primate | TGAGCTTCCCTCCGCCYTRYSVSNVRRRNBNNBNHHNBNBVSWNYTTATAA |
| Alignment | RRYTYNCANHHNBDRRVMBMTTTNWHBDTKABGGTGAYTTCCCABMABVCR |
| consensus | YWGCKMCATGYAAANRKNBHVSRDYSYVNNNNNNNNNNNCHDVNNNNNNNN |
| sequence | NNNNNNNNNNNNNNNCVNNGYGSVCKCKCRYKNNVYKTNNNNCGCNNNSDN |
| 100%_Identity | NNNNNNSNGWYNSNNNRCYCRSGDTTSVNNNNNNCKNGNNNNNNACSTSAR |
| | HNNNNNNNNNHMATGGCG (SEQ ID NO: 789) |

In some embodiments, the promoter comprises a nucleotide sequence having at least 85%, 90%, 95%, 96%, 97%, 98%, 99%, or 100% identity to nucleotides 20-250 of any one of SEQ ID NOs: 784-789 or a functional fragment or variant (e.g., codon optimized) thereof.

### Rodent H1 Promoters

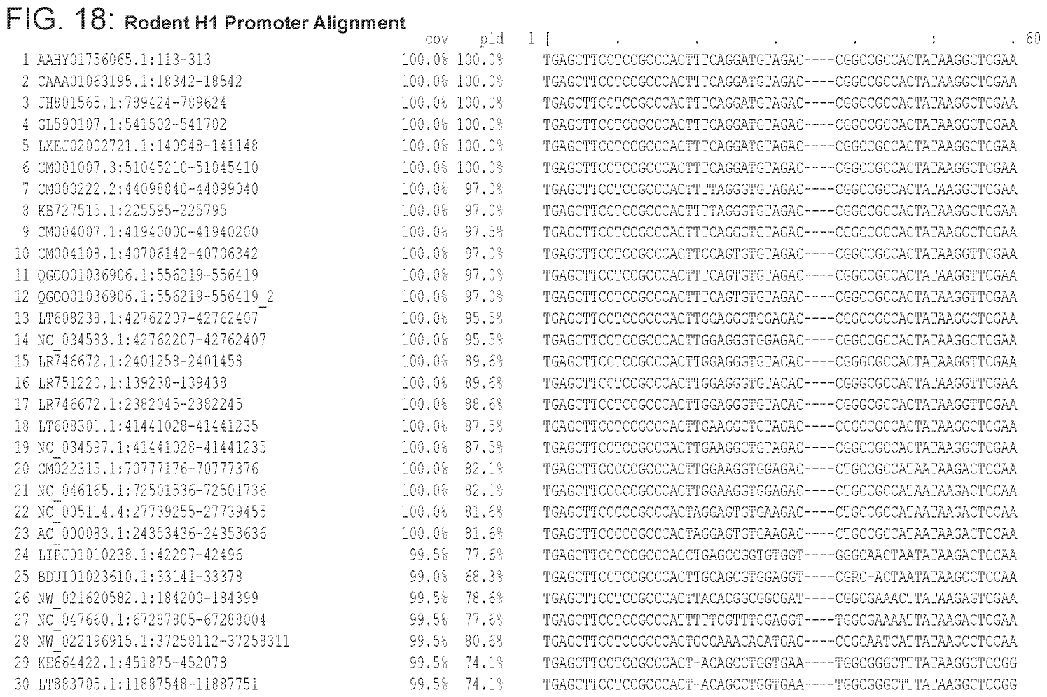

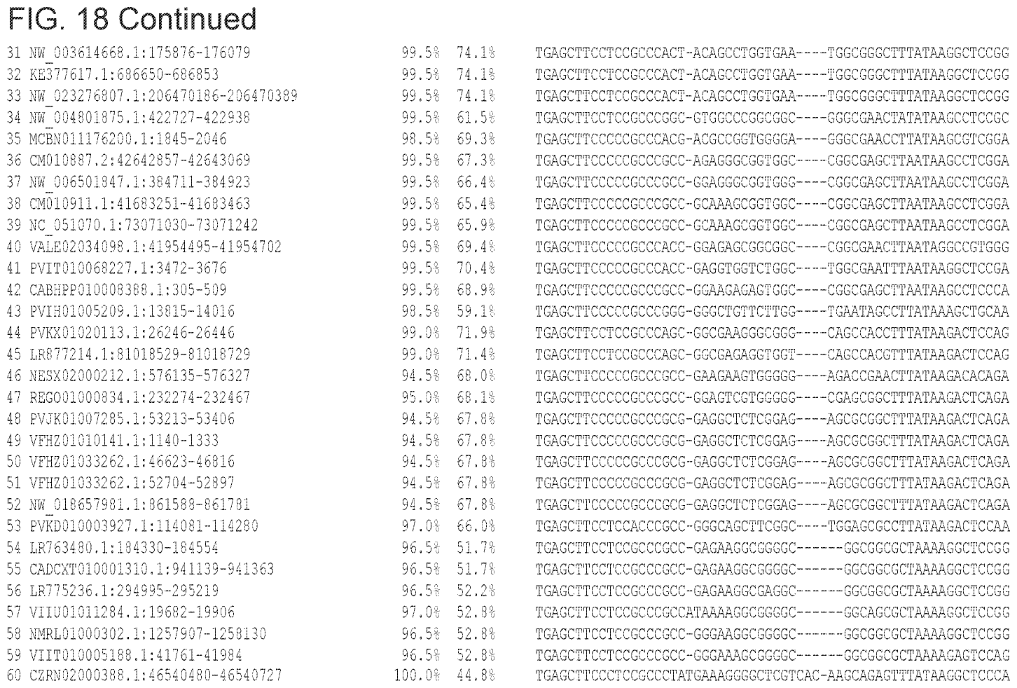

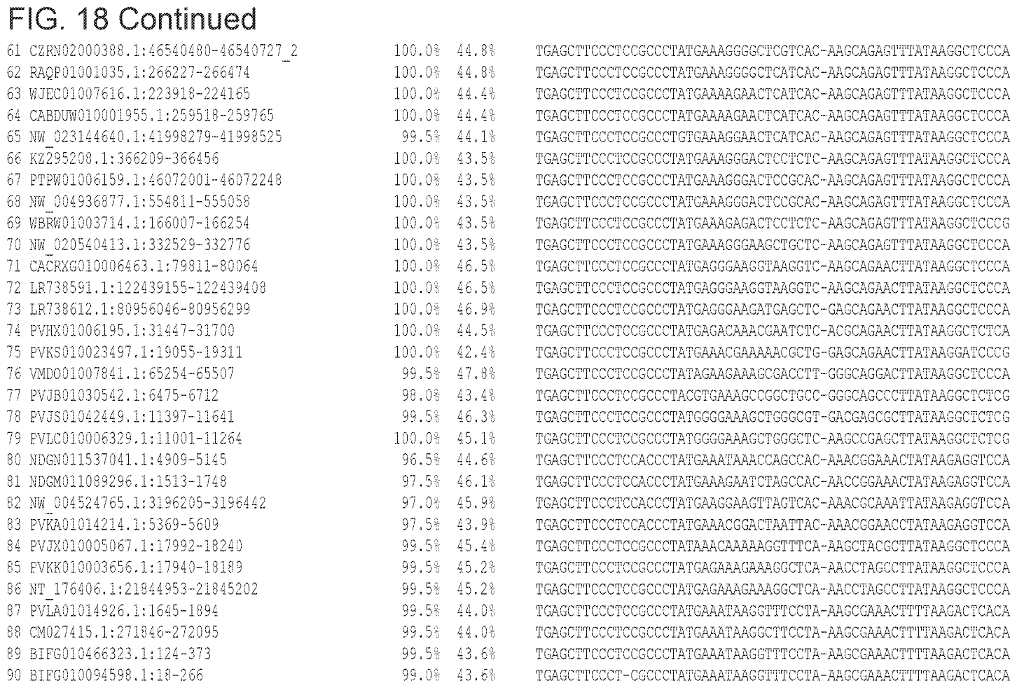

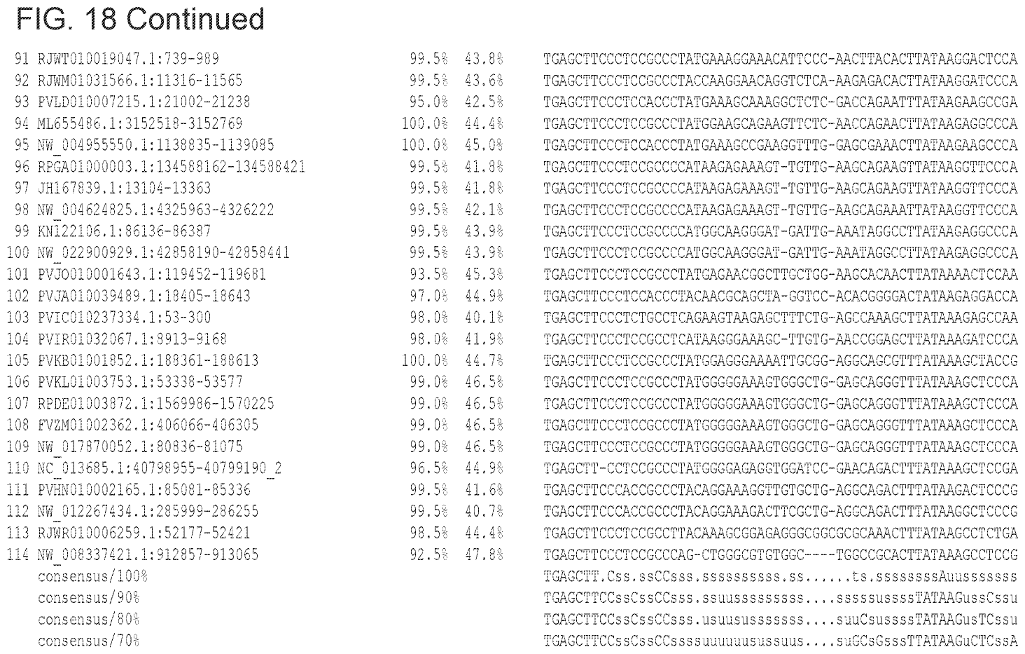

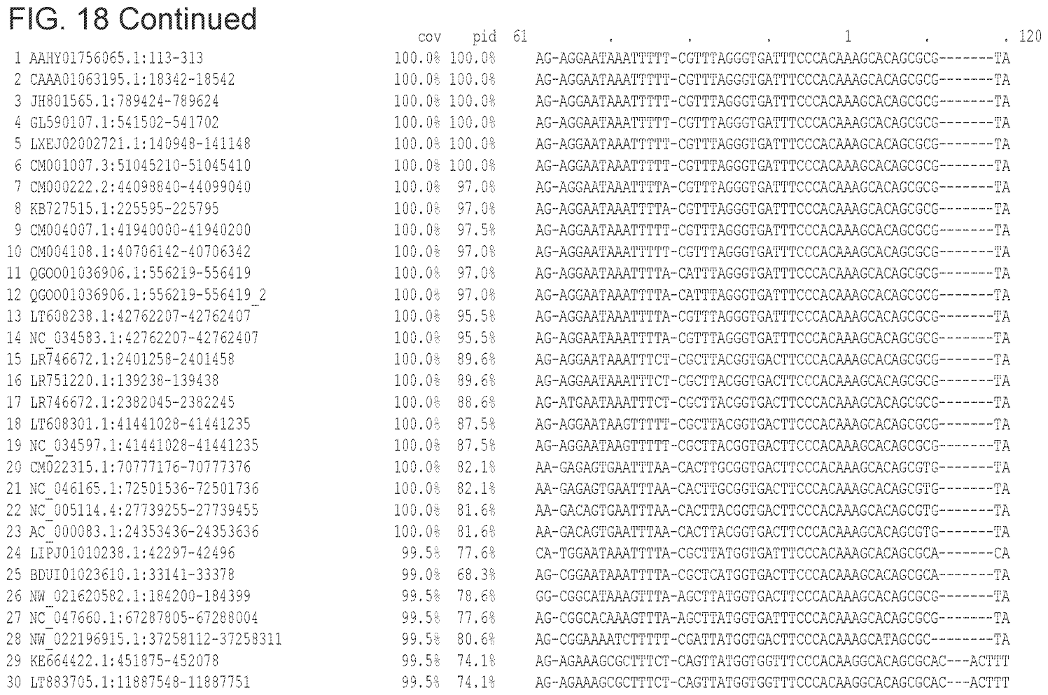

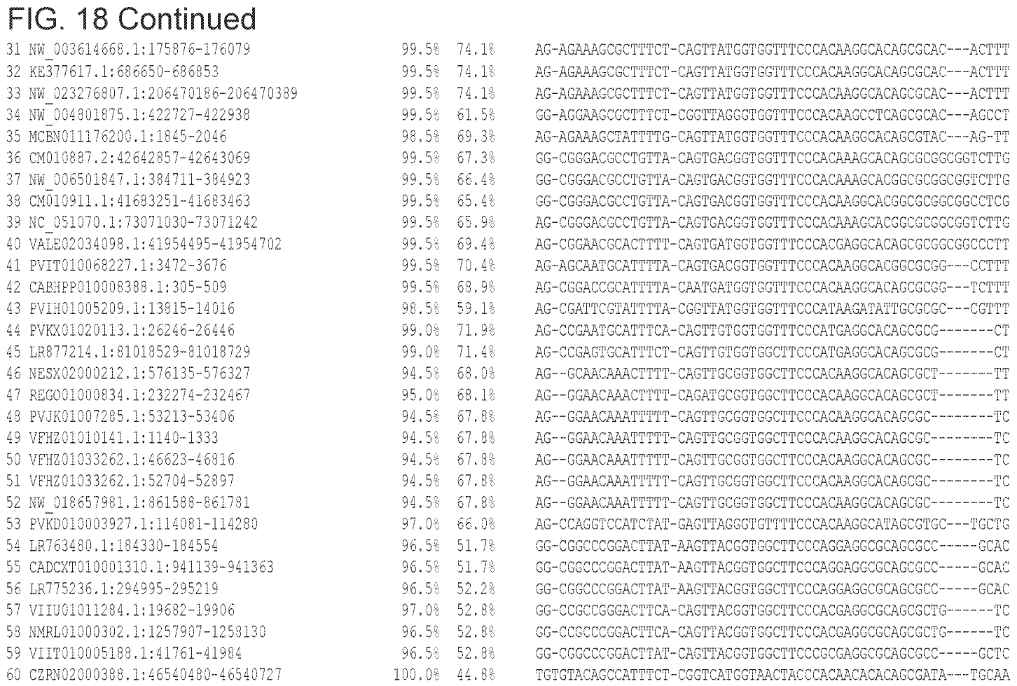

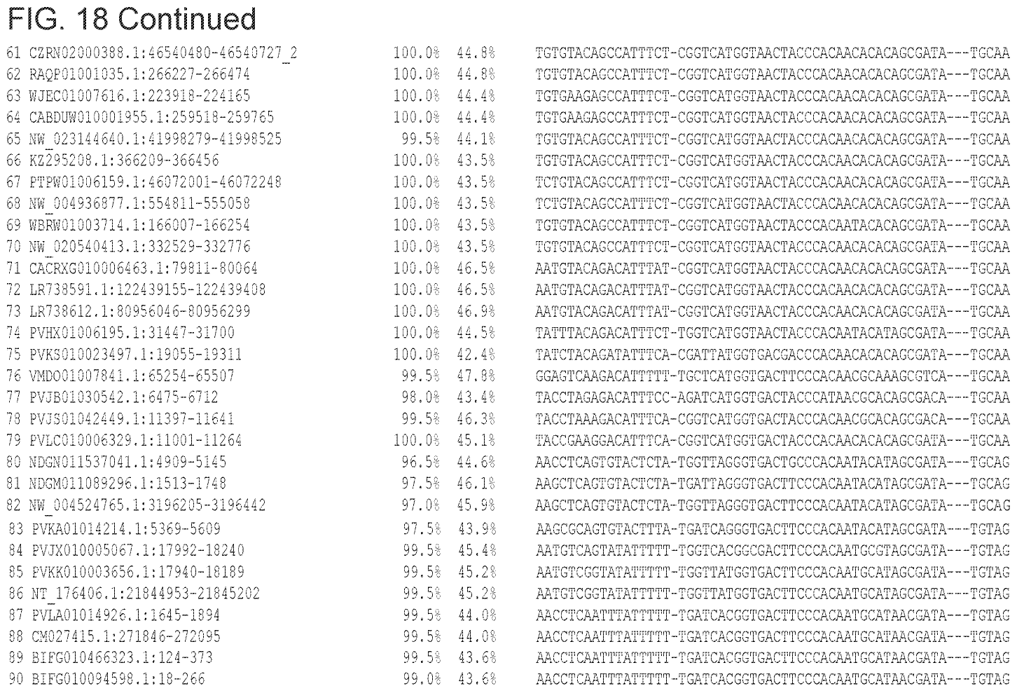

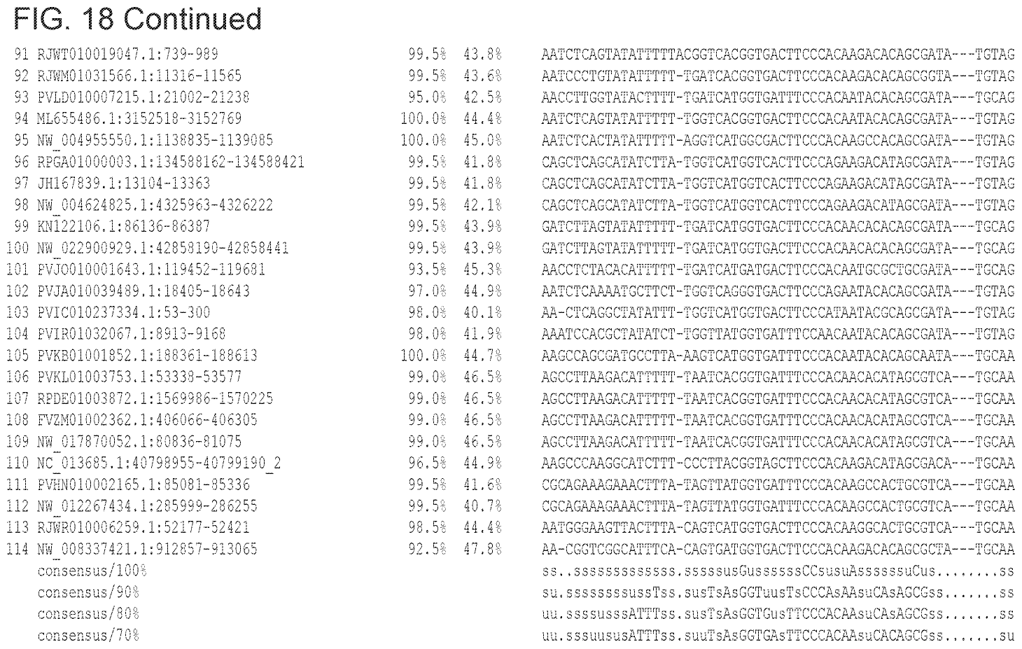

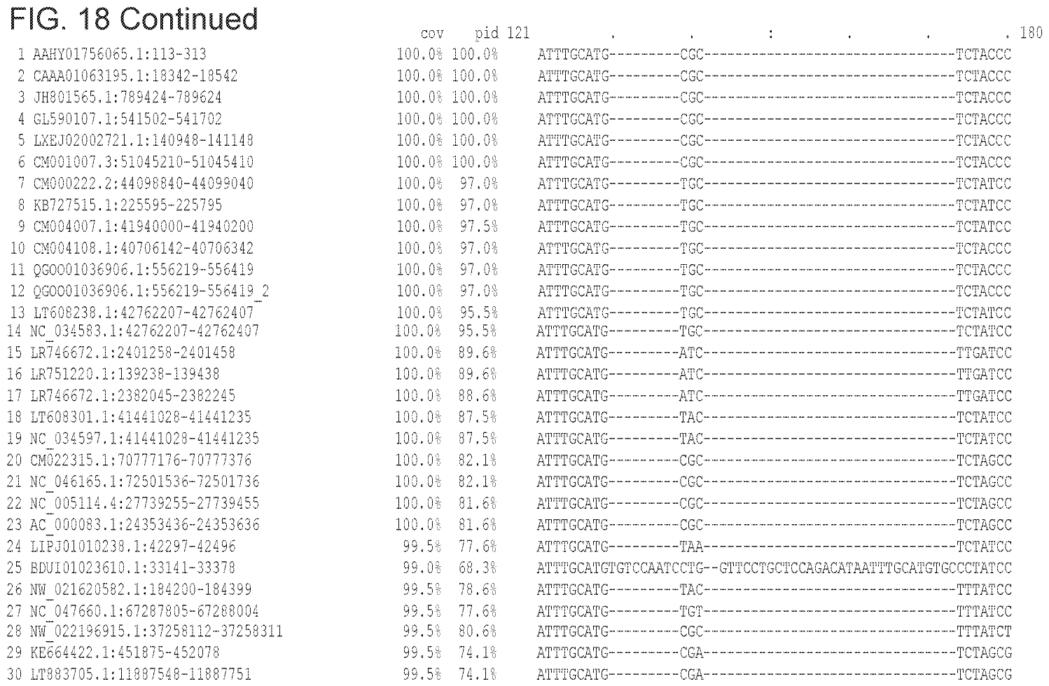

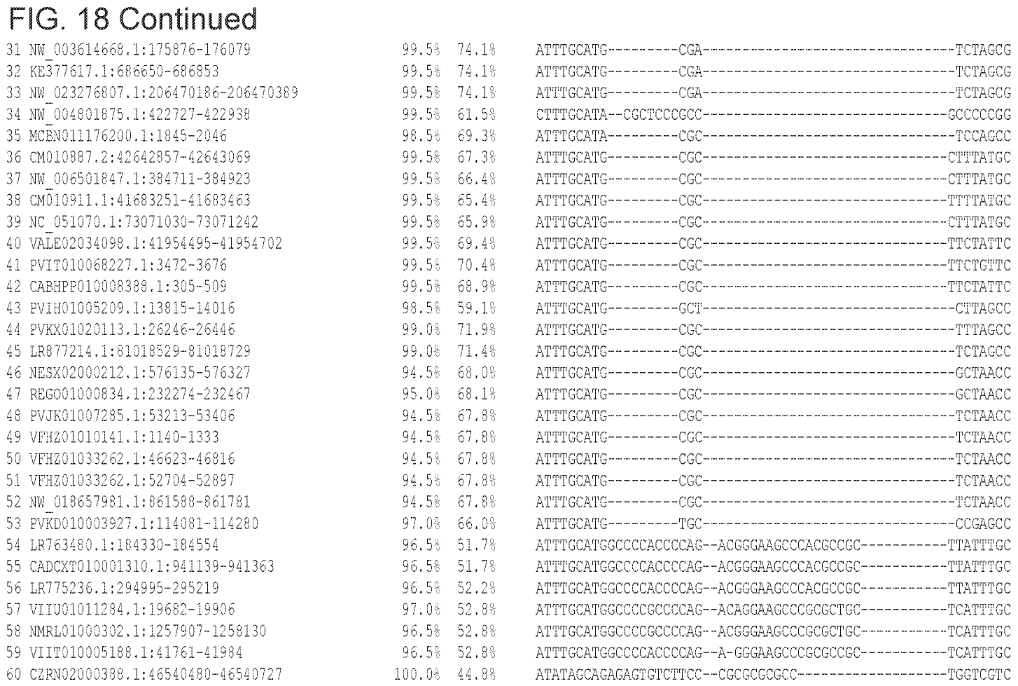

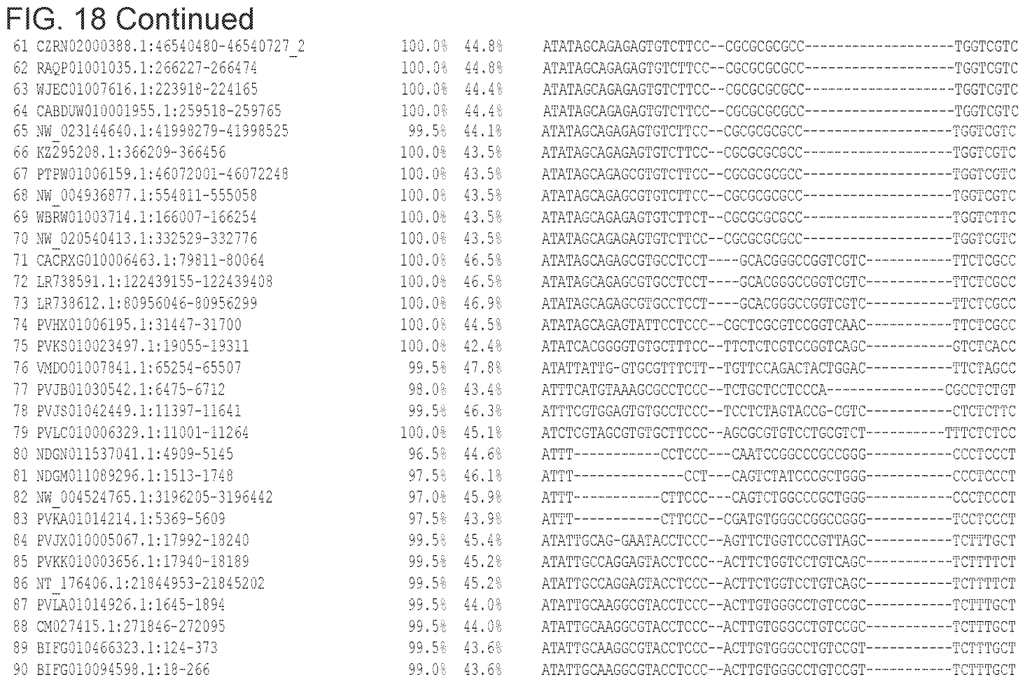

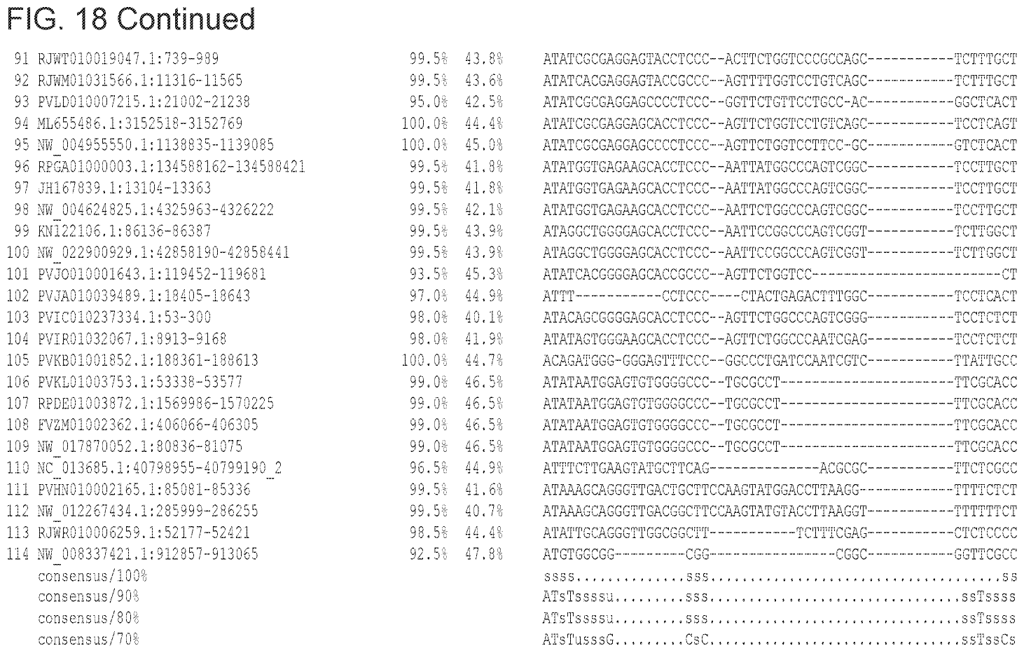

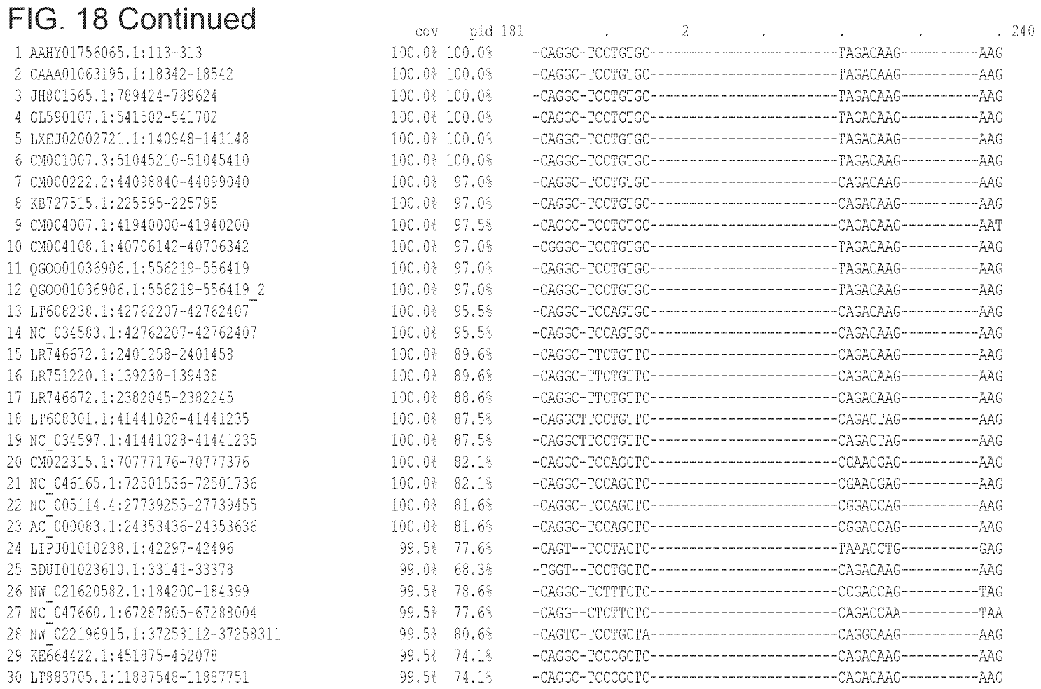

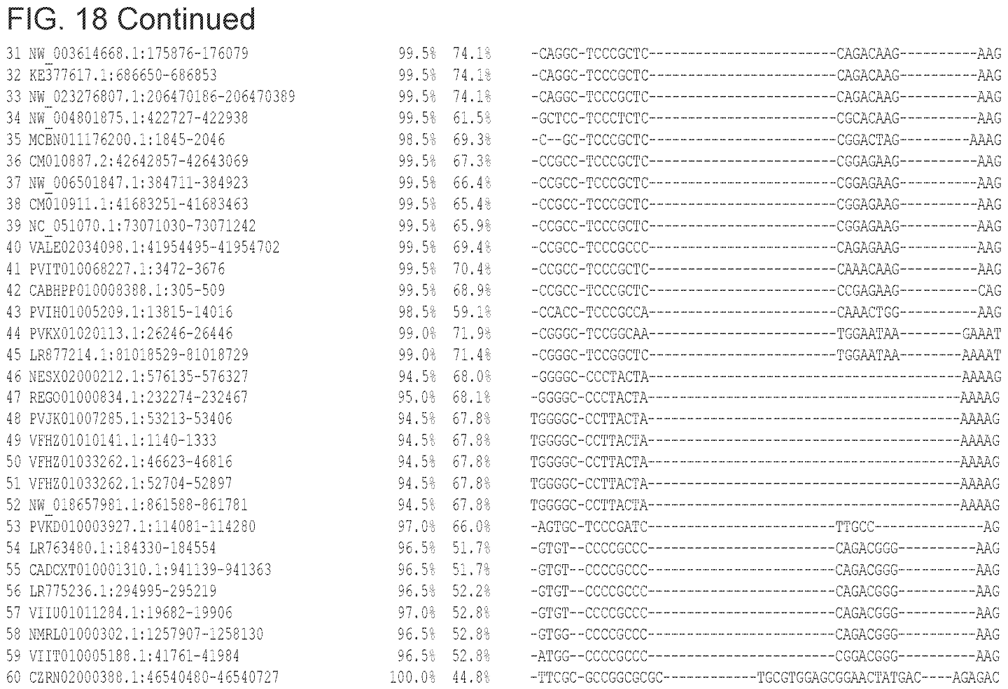

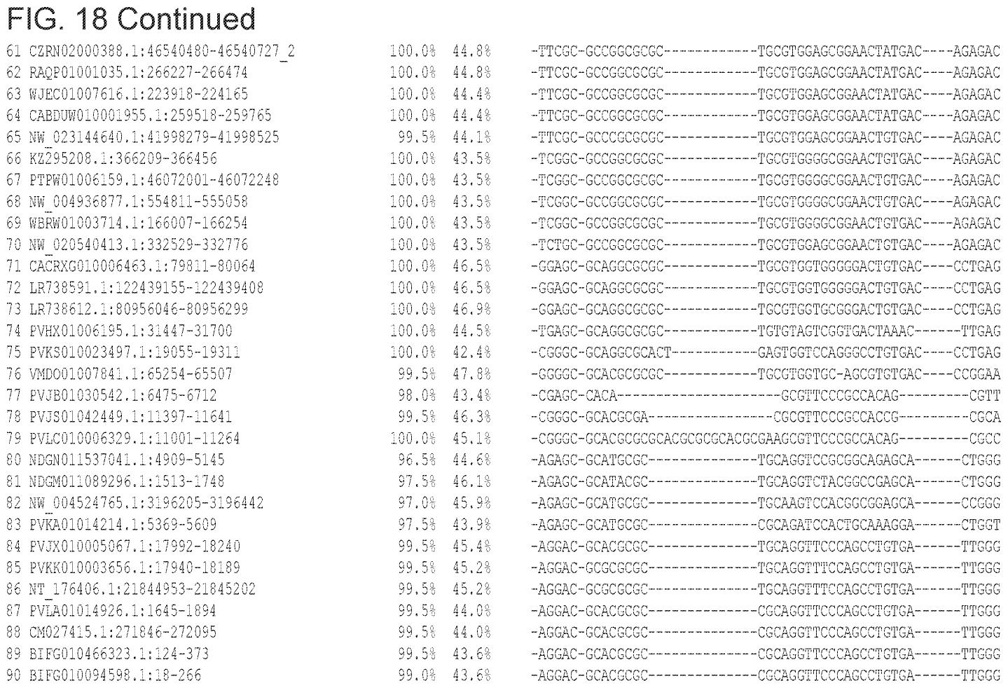

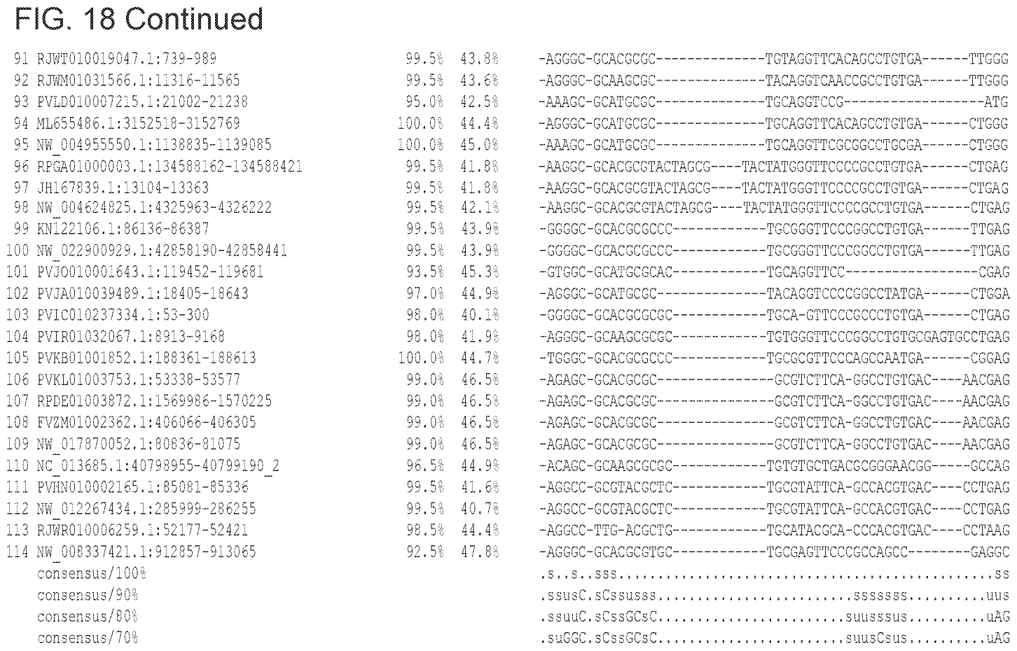

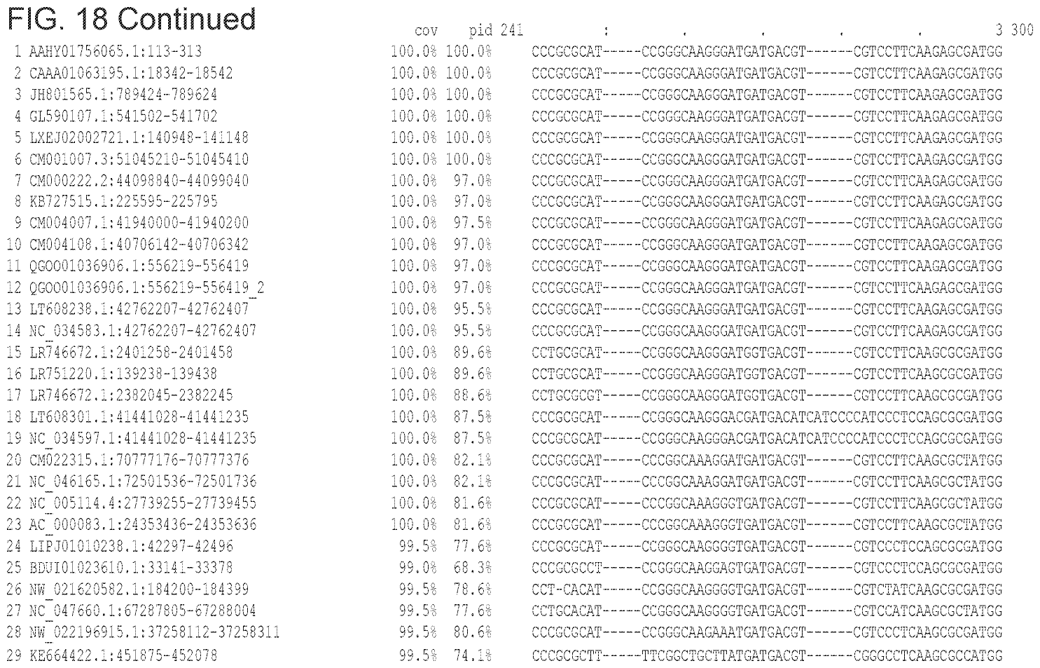

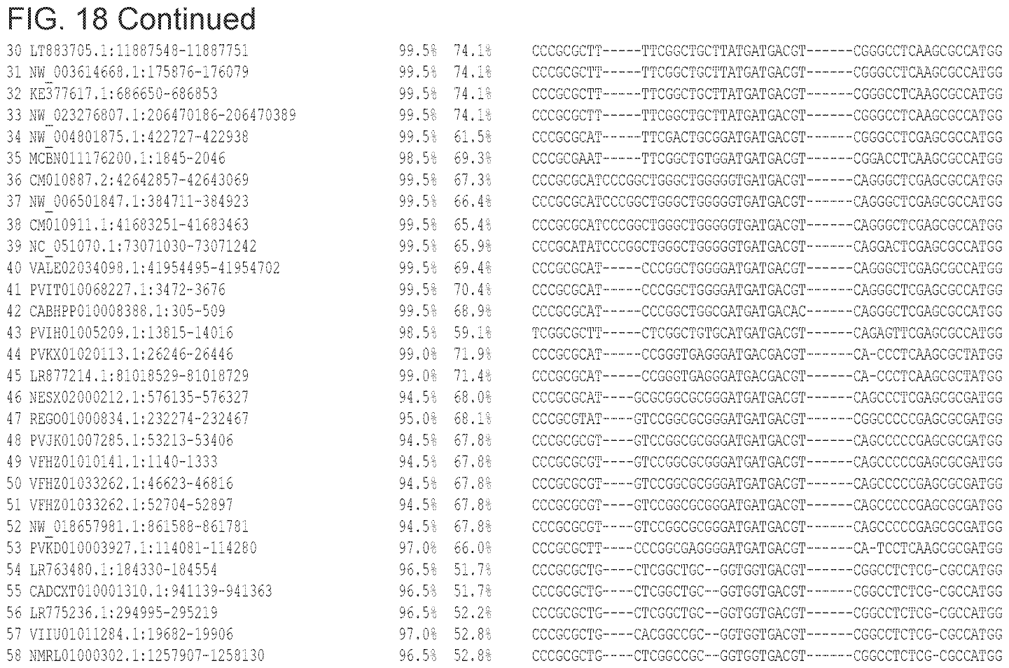

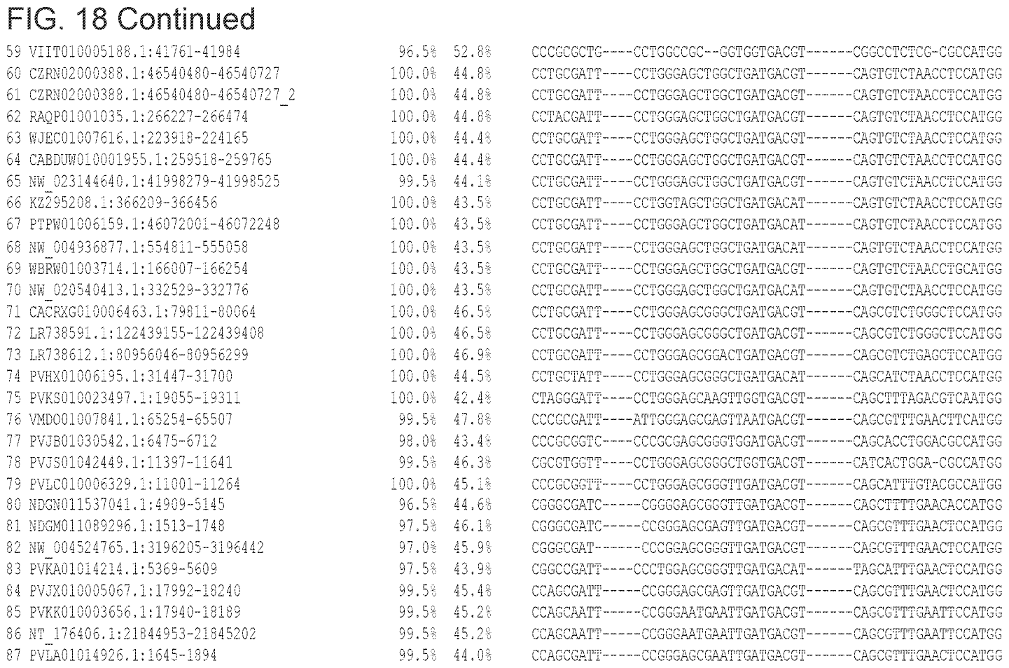

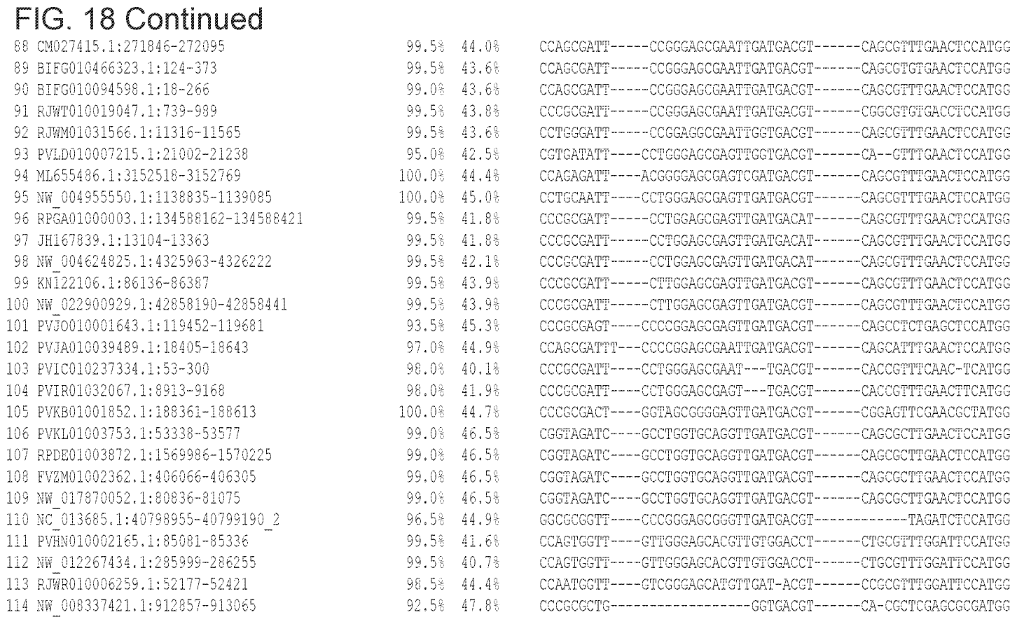

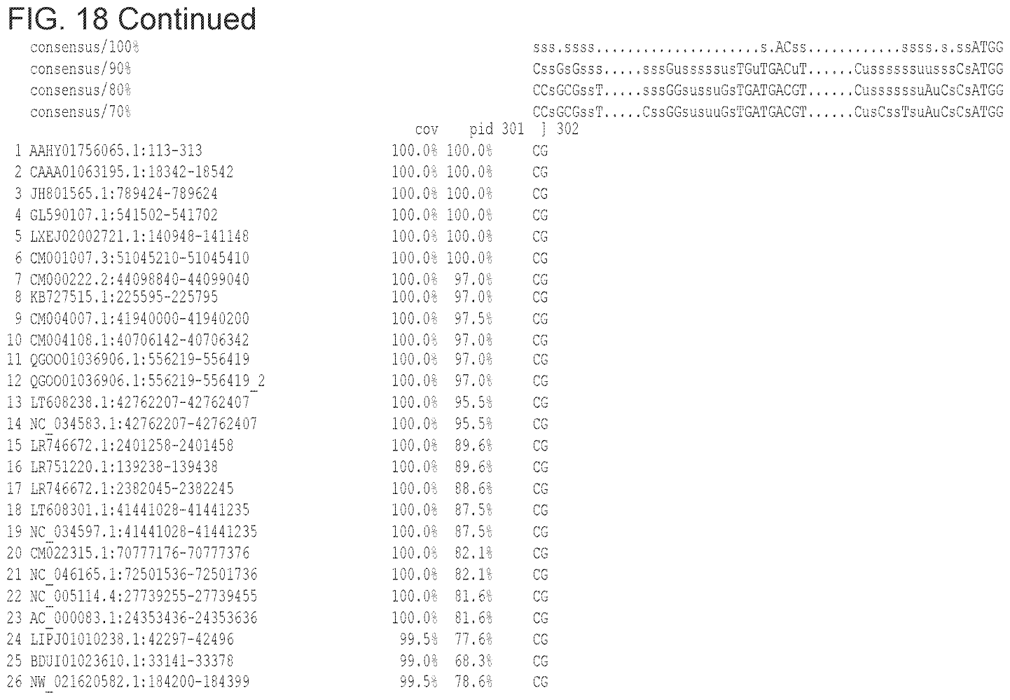

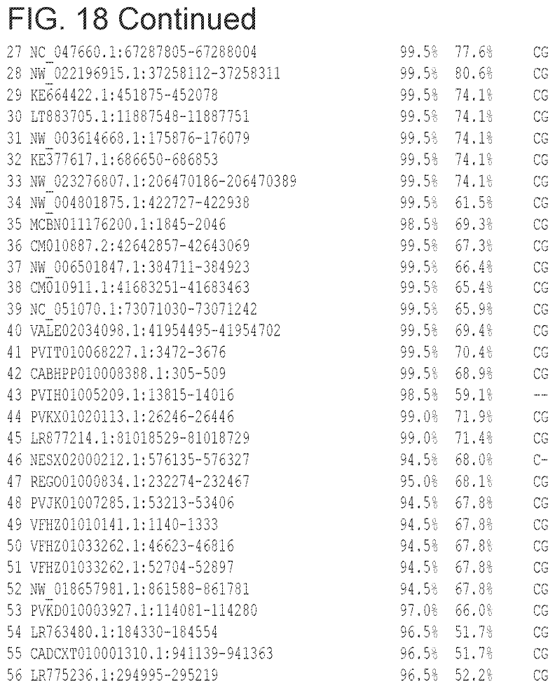

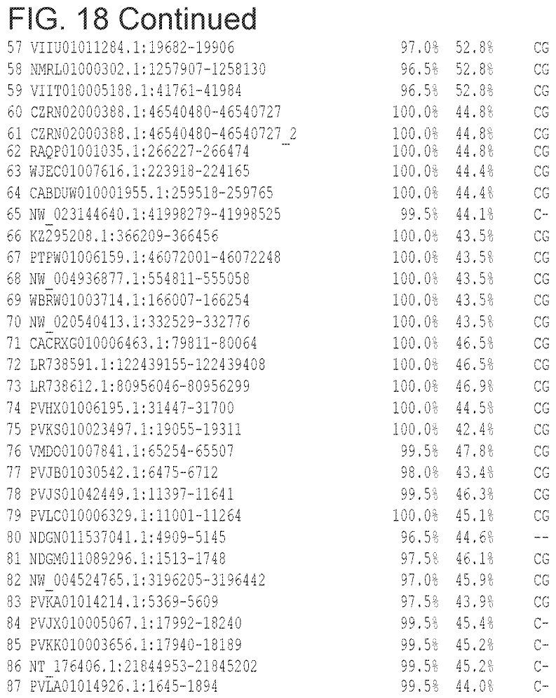

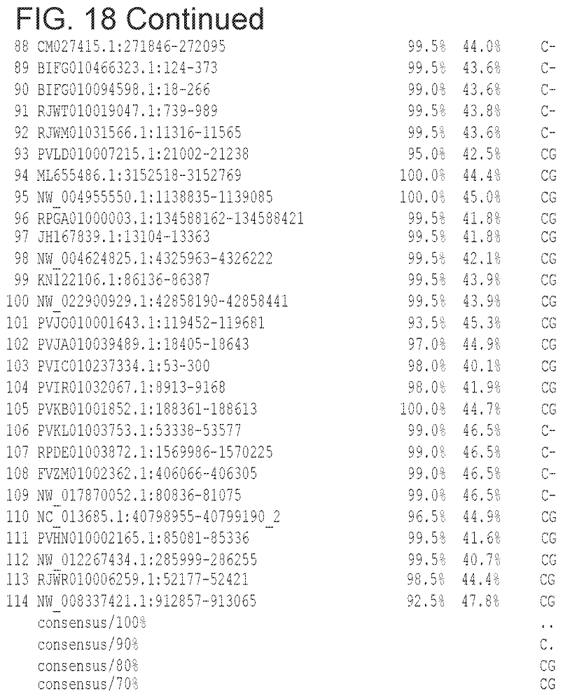

In certain embodiments, the promoter comprises a Rodent H1 promoter. An alignment of Rodent H1 promoter sequences is provided in FIG. 18 (wherein sequences numbered 1-114 in FIG. 18 correspond to SEQ ID NOs: 790-903 or 1859, and consensus/100%, consensus/90%, consensus/80% and consensus/70% correspond to SEQ ID NOs: 1860-1863, respectively). In some embodiments, the promoter comprises a nucleotide sequence having at least 85%, 90%, 95%, 96%, 97%, 98%, 99%, or 100% identity to nucleotides 20-296 any one of the sequences in FIG. 18 or a functional fragment or variant (e.g., codon optimized) thereof.

In certain embodiments, the Rodent H1 promoter a sequence selected from those in TABLE 15.

TABLE 15

| | |
|---|---|
| Rodent | TGAGCTTCCYYCSSCCMYHTRRRRVRDRBDSRBYWSCMRGCVRVMHYTATA |
| Alignment | AGRCTCSMAWRYMKVMRKRHATTTYWAYRVTYAYGGTGRYTTCCCACAAVR |
| consensus | CACAGCGMKACGGTGYWRATWTRSMWGRGHGYRYCKYSCCCMSBKSBNGBC |
| sequence | CDSYCVKSATTTGCATGTBTYYTMDCYTVRGGCTKCMYGCKCRCTAGCGCG |
| 75%_Identity | CATACTGCRKGKYSMSRGMCWRKGACAGTGMNWRAGCCYGCGMWTCCCGSC |
| | YSGGMRMKRGNTGATGACGTCATCCCCRKCSYYYRARCKCSATGGCG |
| | (SEQ ID NO: 904) |
| Rodent | TGAGCTTCCYYCSSCCVYHTRVRRVVDDBDNDBYHVCVRSSVRVVHYTATA |
| Alignment | AGRSTCSVRDRBVKVMRBVHAYTTYWAYRVTYABGGTRRYTWCCCACAANR |
| consensus | CAYAGCGMBVCGGWSYWDATWTVSMDRRSHSYRYYKYVYCCHVBKVBNGBC |
| sequence | CNBBYVKBATTTGCATGTBYYBTHDYYTVVRSCTKCMBGYKCNCWMGCGCG |
| 85%_Identity | CAYRCTGYRKRKHSMSRRMMDRKGACAGTGMNHRRSCCHGCGMWTYCCGSY |
| | YSGGMRVDRRNTGATGACGTCATCCCCRKSSYYYRARMKCSATGGCG |
| | (SEQ ID NO: 905) |
| Rodent | TGAGCTTCCYYCSSCCVYHYDVRRNVNDNDNDBYHVCVRSSVRVVHYTATA |
| Alignment | AGRBKCVVRDRBVBVVVBVNMYYTHWAYRNTYABGGTRRYTWCCCASAANR |
| consensus | CAYAGCGHBVCGGWSYWDATWTVVHDRRSHNYRYYBYVBCCHVBBVNNNBC |
| sequence | CNBBBVDBATTTGCATGTBYBBTHNBYTNNRNCTBCMBRYKMNCWMGCGCG |
| 90%_Identity | CAYRCYRYRBRKHSVBRRMMNRKSACAGTGMNHRRSCSHGMGMWBYCCGSY |
| | YSGGHDVDRRNTGRTGACRTCATCCCCRKBSYYYRRVMKCSATGGCG |
| | (SEQ ID NO: 906) |
| Rodent | TGAGCTTCCYYCSVCCVYNHDNVVNNNNNNNNNBNVCNDVNVRVVNYWAWA |
| Alignment | ARVNKYVVRNRBVNNVVBVNMYBTHWAHRNTBRBGGTRRYTWCCCASRANR |
| consensus | CRYWGCGHNVCGGHSYWNATWKNVHDRRVHNBNBBBYNNCCNVNBNNNNNN |
| sequence | CNNNBNDBATTTGCATGTBBBNKHNBBTNNVNCTBYHNRYBMNCWMGCGCG |
| 95%_Identity | CAYRCYRYRBVKNBVBVVMVNRDSMSAGTGMNHRRBCSNKHRVDBYCCGSY |
| | YBGSHDVNDDNTGRTGACRTCATCCCCRKBVYYYVRVHKCBATGGCG |
| | (SEQ ID NO: 907) |
| Rodent | TGAGCTTCCYHCNVCCNBNNNNVVNNNNNNNNNBNNCNNVNNVVNNHWWWA |
| Alignment | ARVNBHNVRNVNNNNNVNNNVBNYHNAHRNTBRBGGYVRYTWCCCABRANV |
| consensus | CRYDRCGHNVCGGHSYHNATNDNNHNRNVNNNNNBBNNNCCNNNNNNNNNN |
| sequence | HNNNNNNNATTTGCATGTBBBNBNNBBTNNNNCTBYNNDYBHNSWMGCGCG |
| 99%_Identity | CAYRCBRNDNVBNNVBNVVVNVNVVSAGTGMNNNNNBSNDNDNNBYCCGVN |
| | BBGVNDNNNDNYGDBGACVTCATCCCCDBNNHBHVRVHKYBATGGCG |
| | (SEQ ID NO: 908) |
| Rodent | TGAGCTTCCYHCNVCCNNNNNNVNNNNNNNNNNBNNCNNVNNVNNNHWWWA |
| Alignment | RRVNNNNVVNVNNNNNNNNNVBNYHNANVNWBRBGRYVDYKDCCMRBRANV |
| consensus | YDHDRCRNNVCGGHSYHNMYNNNNNNDNVNNNNNBBNNNCCNNNNNNNNNN |
| sequence | HNNNNNNNATTTGCATGTBBBNBNNBBTNNNNCTBHNNDHNHNSWMGCGCG |
| 100%_Identity | CAYRCBRNDNVBNNVBNVVVNNNVVSAGTGMNNNNNBBNNNDNNBYCCGVN |
| | BNSNNDNNNNNBRDBGACVYCATCCCYNBNNHBNVDNNDBNATGGCG |
| | (SEQ ID NO: 909) |

In some embodiments, the promoter comprises a nucleotide sequence having at least 85%, 90%, 95%, 96%, 97%, 98%, 99%, or 10000 identity to nucleotides 20-296 of any one of SEQ ID NOs: 904-909 or a functional fragment or variant (e.g., codon optimized) thereof.

### Xenarthra H1 Promoters

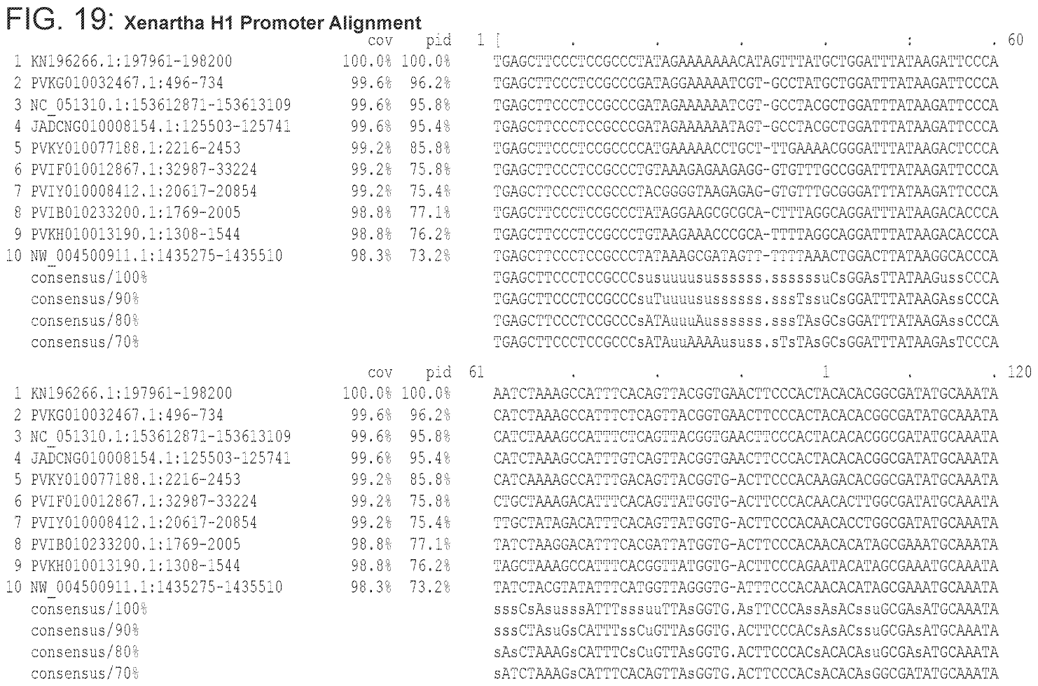

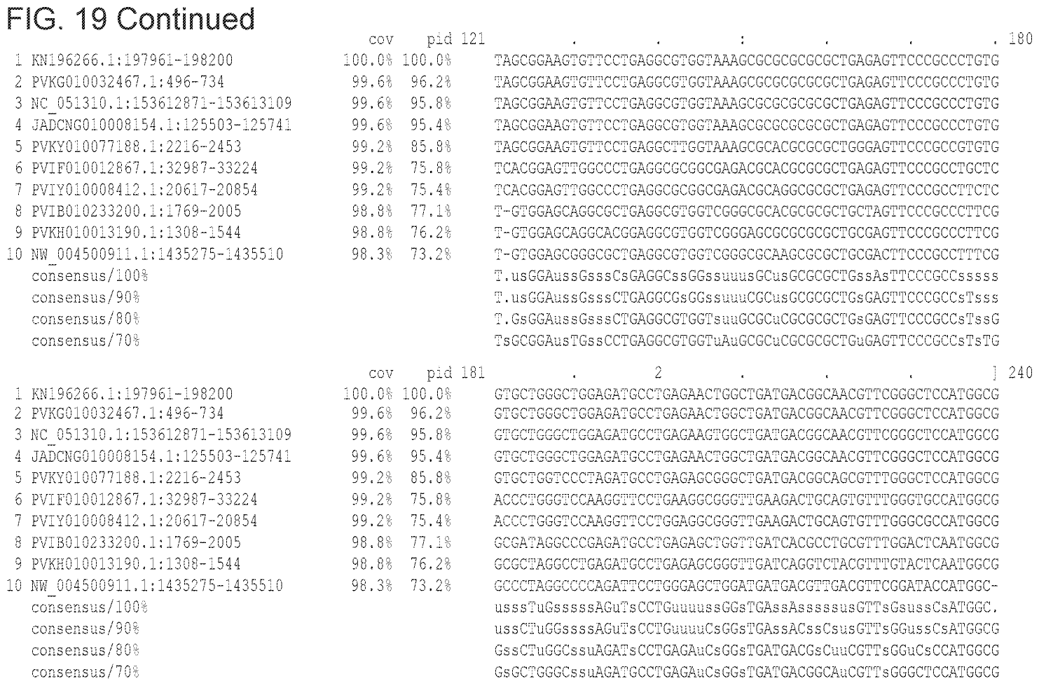

In certain embodiments, the promoter comprises an Xenarthra H1 promoter. An alignment of Xenarthra H1 promoter sequences is provided in FIG. 19 (wherein sequences numbered 1-10 in FIG. 19 correspond to SEQ ID NOs: 910-919, and consensus/100%, consensus/90%, consensus/80% and consensus/70% correspond to SEQ ID NOs: 1864-1867, respectively). In some embodiments, the promoter comprises a nucleotide sequence having at least 85%, 90%, 95%, 96%, 97%, 98%, 99%, or 100% identity to nucleotides 20-234 of any one of the sequences in FIG. 19 or a functional fragment or variant (e.g., codon optimized) thereof.

In certain embodiments, the Xenarthra H1 promoter comprises a sequence selected from those in TABLE 16.

TABLE 16

| Xenarthra | TGAGCTTCCCTCCGCCCKATARRRARMVHSVDKYBTANGCDGGATTTATAA |
|---|---|
| Alignment | GAYWCCCAYAKCTAAAGMCATTTCWCRGTTAYGGTGNACTTCCCACWACAC |
| consensus | AYRGCGAWATGCAAATATNGYGGARSWGKYSCTGAGGCGTGGTMRRGCGCR |
| sequence | CGCGCGCTGMGAGTTCCCGCCYTKYGGYSCTRGGCYSRAGATKCCTGAGAR |
| 75%_Identity | CKGGYTGATGACGKCWRCGTTYGGRCKCCATGGCG (SEQ ID NO: |
| | 920) |
| Xenarthra | TGAGCTTCCCTCCGCCCKRTRRRRHRMVHVVDKYBTWNRCDGGATTTATAA |
| Alignment | GAYWCCCAYWKCTAHRGMCATTTSWCRGTTAYGGTGNACTTCCCACWABAC |
| consensus | HYRGCGAWATGCAAATATNRYGGARBWGKYSCTGAGGCGYGGYVRRRCGCR |
| sequence | VGCGCGCTGMGAGTTCCCGCCYTBYSRYSCTRGGYYSNAGRTKCCTGRRRR |
| 85%_Identity | CKGGYTGAWSACKKCWRYGTTYGGRYKCMATGGCG (SEQ ID NO: |
| | 921) |
| Xenarthra | TGAGCTTCCCTCCGCCCKRTRRRRHRMVHVVDKYBTWNRCDGGATTTATAA |
| Alignment | GAYWCCCAYWKCTAHRGMCATTTSWCRGTTAYGGTGNACTTCCCACWABAC |
| consensus | HYRGCGAWATGCAAATATNRYGGARBWGKYSCTGAGGCGYGGYVRRRCGCR |
| sequence | VGCGCGCTGMGAGTTCCCGCCYTBYSRYSCTRGGYYSNAGRTKCCTGRRRR |
| 90%_Identity | CKGGYTGAWSACKKCWRYGTTYGGRYKCMATGGCG (SEQ ID NO: |
| | 922) |
| Xenarthra | TGAGCTTCCCTCCGCCCBRYRRRRHRMNNVNDNBYBWWNRCNGGAYTTATA |
| Alignment | AGRYWCCCAHWKCWAHRKMYATTTSWYRRTTABGGTGNAYTTCCCASWABA |
| consensus | CHYRGCGAWATGCAAATATNRYGGARBDGKYVCKGAGGCKYGGYVRRRMGC |
| sequence | RVGCGCGCTGVKASTTCCCGCCBKBYSRYSMTRGKYYBNAGRTKCCTGRRR |
| 95%_Identity | RSKGGHTGAWSASKBYDRYGTTYGKRYDCMATGGCG (SEQ ID NO: |
| | 923) |
| Xenarthra | TGAGCTTCCCTCCGCCCBRYRRRRHRMNNVNDNBYBWWNRCNGGAYTTATA |
| Alignment | AGRYWCCCAHWKCWAHRKMYATTTSWYRRTTABGGTGNAYTTCCCASWABA |
| consensus | CHYRGCGAWATGCAAATATNRYGGARBDGKYVCKGAGGCKYGGYVRRRMGC |
| sequence | RVGCGCGCTGVKASTTCCCGCCBKBYSRYSMTRGKYYBNAGRTKCCTGRRR |
| 99%_Identity | RSKGGHTGAWSASKBYDRYGTTYGKRYDCMATGGCG (SEQ ID NO: |
| | 924) |
| Xenarthra | TGAGCTTCCCTCCGCCCBRYRRRRHRMNNVNDNBYBWWNRCNGGAYTTATA |
| Alignment | AGRYWCCCAHWKCWAHRKMYATTTSWYRRTTABGGTGNAYTTCCCASWABA |
| consensus | CHYRGCGAWATGCAAATATNRYGGARBDGKYVCKGAGGCKYGGYVRRRMGC |
| sequence | RVGCGCGCTGVKASTTCCCGCCBKBYSRYSMTRGKYYBNAGRTKCCTGRRR |
| 100%_Identity | RSKGGHTGAWSASKBYDRYGTTYGKRYDCMATGGCG (SEQ ID NO: |
| | 925) |

In some embodiments, the promoter comprises a nucleotide sequence having at least 85%, 90%, 95%, 96%, 97%, 98%, 99%, or 100% identity to nucleotides 20-233 of any one of SEQ ID NOs: 920-925 or a functional fragment or variant (e.g., codon optimized) thereof.

### Gar1 Promoters

A custom perl script was developed to compare the 5′ transcriptional start sites of pol III genes with that of pol II genes. The results were filtered for those that are orientated in opposite directions (divergent transcription). One compact bidirectional promoter identified using this method was the Gar1 promoter. On one side, the GAR1 promoter expresses the GAR1 protein, which is involved with snoRNAs, rRNA processing, and telomerase activity. The GAR1 protein appears to be expressed in all tissues, suggesting that the GAR1 promoter can drive expression ubiquitously (on the world wide web at proteinatlas.org/ENSG00000109534-GAR1/tissue). On the other side, it expresses a lncRNA (AC126283.1 or ENSG00000272795) with unknown function, and high expression in the testis.

Accordingly in certain embodiments, the promoter is a Gar1 promoter. In certain embodiments, the Gar1 promoter is a mammalian promoter, e.g., a human Gar1 promoter, a carnivora Gar1 promoter, a primate Gar1 promoter, or a rodent Gar1 promoter. In some embodiments, the Gar1 promoter comprises a nucleotide sequence having at least 85%, 90%, 95%, 96%, 97%, 98%, 99%, or 100% identity of any one of SEQ ID NOs: 107-203 or a codon-optimized variant and/or fragment thereof. In some embodiments, the promoter comprises the nucleotide sequence of any one of SEQ ID NOs: 107-203 or a codon-optimized variant and/or fragment thereof.

In certain embodiments, a functional fragment comprises a truncation of from about 10 bases to about 70 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 10 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 20 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 30 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 40 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 50 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 60 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 70 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the Gar promoter comprises a TATA mutation. In certain embodiments, the TATA mutation is a TATAA→TCGAA mutation.

In certain embodiments, a nucleic acid comprising a Gar1 promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a Gar1 promoter described herein further comprises a terminator sequence. In certain embodiments, the terminator sequence comprises one of the terminator sequences in TABLE 17.

TABLE 17

| | |
|---|---|
| a synthetic poly(A) | AATAAAATATCTTTATTTTCATTACATCTGTGTGTTGGTTTTTT |
| sequence (SPA) | GTGTG (SEQ ID NO: 258) |
| SPA and Pause | AATAAAATATCTTTATTTTCATTACATCTGTGTGTTGGTTTTTT |
| | GTGTGAATCGATAGTACTAACATACGCTCTCCATCAAAACAA |
| | AACGAAACAAAACAAACTAGCAAAATAGGCTGTCCCCAGTG |
| | CAAGTGCAGGTGCCAGAACATTTCTCT (SEQ ID NO: 259); |
| SV40 (240 bp) | ATCTAGATAACTGATCATAATCAGCCATACCACATTTGTAGA |
| | GGTTTTACTTGCTTTAAAAAACCTCCCACACCTCCCCCTGAAC |
| | CTGAAACATAAAATGAATGCAATTGTTGTTGTTAACTTGTTTA |
| | TTGCAGCTTATAATGGTTACAAATAAAGCAATAGCATCACAA |
| | ATTTCACAAATAAAGCATTTTTTTCACTGCATTCTAGTTGTGG |
| | TTTGTCCAAACTCATCAATGTATCTTA (SEQ ID NO: 260) |
| SV 40-mini | TTGTTTATTGCAGCTTATAATGGTTACAAATAAAGCAATAGC |
| (120 bp) | ATCACAAATTTCACAAATAAAGCATTTTTTTCACTGCATTCTA |
| | GTTGTGGTTTGTCCAAACTCATCAATGTATCTTAT (SEQ ID |
| | NO: 261) |
| bGH poly A | CGACTGTGCCTTCTAGTTGCCAGCCATCTGTTGTTTGCCCCTC |
| | CCCCGTGCCTTCCTTGACCCTGGAAGGTGCCACTCCCACTGTC |
| | CTTTCCTAATAAAATGAGGAAATTGCATCGCATTGTCTGAGT |
| | AGGTGTCATTCTATTCTGGGGGGTGGGGTGGGGCAGGACAGC |
| | AAGGGGGAGGATTGGGAAGACAATAGCAGGCATGCTGGGGA |
| | TGCGGTGGGCTCTATGG (SEQ ID NO: 262) |
| TKpoly A | GGGGGAGGCTAACTGAAACACGGAAGGAGACAATACCGGAA |
| | GGAACCCGCGCTATGACGGCAATAAAAAGACAGAATAAAAC |
| | GCACGGGTGTTGGGTCGTTTGTTCATAAACGCGGGGTTCGGT |
| | CCCAGGGCTGGCACTCTGTCGATACCCCACCGAGACCCCATT |
| | GGGGCCAATACGCCCGCGTTTCTTCCTTTTCCCCACCCCACCC |
| | CCCAAGTTCGGGTGAAGGCCCAGGGCTCGCAGCCAACGTCG |
| | GGGCGGCAGGCCCTGCCATAG (SEQ ID NO: 263) |
| sNRP1 | GGTATCAAATAAAATACGAAATGTGACAGATT (SEQ ID NO: |
| | 264) |
| sNRP1a | AAATAAAATACGAAATGTGACAGATT (SEQ ID NO: 265) |
| Histone H4B | GGTTGCTGATTTCTCCACAGCTTGCATTTCTGAACCAAAGGCC |
| | CTTTTCAGGGCCGCCCAACTAAACAAAAGAAGAGCTGTATCC |
| | ATTAAGTCAAGAAGC (SEQ ID NO: 266) |
| MALAT-1 | GATTCGTCAGTAGGGTTGTAAAGGTTTTTCTTTTCCTGAGAAA |
| | ACAACCTTTTGTTTTCTCAGGTTTTGCTTTTTGGCCTTTCCCTA |
| | GCTTTAAAAAAAAAAAAGCAAAAGACGCTGGTGGCTGGCAC |
| | TCCTGGTTTCCAGGACGGGGTTCAAGTCCCTGCGGTGTCTTTG |
| | CTT (SEQ ID NO: 267) |
| MALAT-comp14 | AAAGGTTTTTCTTTTCCTGAGAAATTTCTCAGGTTTTGCTTTTT |
| | AAAAAAAAAGCAAAAGACGCTGGTGGCTGGCACTCCTGGTT |
| | TCCAGGACGGGGTTCAAGTCCCTGCGGTGTCTTTGCTT (SEQ |
| | ID NO: 268) |

In certain embodiments, the Gar1 promoter is coupled with a viral intron (e.g., an SV40i intron, a MVM intron, a Mv2 intron, an HNRNPH1 intron, chimeric introns or synthetic introns).

In certain embodiments, the Gar1 promoter does not comprise a viral promoter and/or a synthetic promoter. In certain embodiments, the compact promoter does not comprise F5tg83.

In certain embodiments, the Gar1 promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a Gar1 promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### Other Bidirectional Promoters

Using the custom perl script described above, additional bidirectional promoters were identified that can be used according to the methods described herein. In certain embodiments, the promoter is a bidirectional promoter comprising a nucleotide sequence having at least 85%, 90%, 95%, 96%, 97%, 98%, 99%, or 100% identity of any one of SEQ ID NOs: 204-255 or a codon-optimized variant and/or fragment thereof. In some embodiments, the bidirectional promoter comprises the nucleotide sequence of any one of SEQ ID NOs: 204-255 or a codon-optimized variant and/or fragment thereof.

In certain embodiments, a functional fragment comprises a truncation of from about 10 bases to about 70 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 10 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 20 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 30 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 40 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 50 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 60 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 70 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the promoter comprises a TATA mutation. In certain embodiments, the TATA mutation is a TATAA→TCGAA mutation.

In certain embodiments, the promoter is not one or more of SEQ ID NO: 241-SEQ ID NO: 255.

In certain embodiments, a nucleic acid comprising a bidirectional promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a bidirectional promoter described herein further comprises a terminator sequence. In certain embodiments, the terminator sequence comprises one of the terminator sequences in TABLE 18.

TABLE 18

| | |
|---|---|
| a synthetic poly(A) | AATAAAATATCTTTATTTTCATTACATCTGTGTGTTGGTTTTTT |
| sequence (SPA) | GTGTG (SEQ ID NO: 258) |
| SPA and Pause | AATAAAATATCTTTATTTTCATTACATCTGTGTGTTGGTTTTTT |
| | GTGTGAATCGATAGTACTAACATACGCTCTCCATCAAAACAA |
| | AACGAAACAAAACAAACTAGCAAAATAGGCTGTCCCCAGTG |
| | CAAGTGCAGGTGCCAGAACATTTCTCT (SEQ ID NO: 259); |
| SV40 (240 bp) | ATCTAGATAACTGATCATAATCAGCCATACCACATTTGTAGA |
| | GGTTTTACTTGCTTTAAAAAACCTCCCACACCTCCCCCTGAAC |
| | CTGAAACATAAAATGAATGCAATTGTTGTTGTTAACTTGTTTA |
| | TTGCAGCTTATAATGGTTACAAATAAAGCAATAGCATCACAA |
| | ATTTCACAAATAAAGCATTTTTTTCACTGCATTCTAGTTGTGG |
| | TTTGTCCAAACTCATCAATGTATCTTA (SEQ ID NO: 260) |
| SV 40-mini | TTGTTTATTGCAGCTTATAATGGTTACAAATAAAGCAATAGC |
| (120 bp) | ATCACAAATTTCACAAATAAAGCATTTTTTTCACTGCATTCTA |
| | GTTGTGGTTTGTCCAAACTCATCAATGTATCTTAT (SEQ ID |
| | NO: 261) |
| bGH poly A | CGACTGTGCCTTCTAGTTGCCAGCCATCTGTTGTTTGCCCCTC |
| | CCCCGTGCCTTCCTTGACCCTGGAAGGTGCCACTCCCACTGTC |
| | CTTTCCTAATAAAATGAGGAAATTGCATCGCATTGTCTGAGT |
| | AGGTGTCATTCTATTCTGGGGGGTGGGGTGGGGCAGGACAGC |
| | AAGGGGGAGGATTGGGAAGACAATAGCAGGCATGCTGGGGA |
| | TGCGGTGGGCTCTATGG (SEQ ID NO: 262) |
| TKpoly A | GGGGGAGGCTAACTGAAACACGGAAGGAGACAATACCGGAA |
| | GGAACCCGCGCTATGACGGCAATAAAAAGACAGAATAAAAC |
| | GCACGGGTGTTGGGTCGTTTGTTCATAAACGCGGGGTTCGGT |
| | CCCAGGGCTGGCACTCTGTCGATACCCCACCGAGACCCCATT |
| | GGGGCCAATACGCCCGCGTTTCTTCCTTTTCCCCACCCCACCC |
| | CCCAAGTTCGGGTGAAGGCCCAGGGCTCGCAGCCAACGTCG |
| | GGGCGGCAGGCCCTGCCATAG (SEQ ID NO: 263) |
| sNRP1 | GGTATCAAATAAAATACGAAATGTGACAGATT (SEQ ID NO: |
| | 264) |
| SNRP1a | AAATAAAATACGAAATGTGACAGATT (SEQ ID NO: 265) |
| Histone H4B | GGTTGCTGATTTCTCCACAGCTTGCATTTCTGAACCAAAGGCC |
| | CTTTTCAGGGCCGCCCAACTAAACAAAAGAAGAGCTGTATCC |
| | ATTAAGTCAAGAAGC (SEQ ID NO: 266) |
| MALAT-1 | GATTCGTCAGTAGGGTTGTAAAGGTTTTTCTTTTCCTGAGAAA |
| | ACAACCTTTTGTTTTCTCAGGTTTTGCTTTTTGGCCTTTCCCTA |
| | GCTTTAAAAAAAAAAAAGCAAAAGACGCTGGTGGCTGGCAC |
| | TCCTGGTTTCCAGGACGGGGTTCAAGTCCCTGCGGTGTCTTTG |
| | CTT (SEQ ID NO: 267) |
| MALAT-comp14 | AAAGGTTTTTCTTTTCCTGAGAAATTTCTCAGGTTTTGCTTTTT |
| | AAAAAAAAAGCAAAAGACGCTGGTGGCTGGCACTCCTGGTT |
| | TCCAGGACGGGGTTCAAGTCCCTGCGGTGTCTTTGCTT (SEQ |
| | ID NO: 268) |

In certain embodiments, the bidirectional promoter is coupled with a viral intron (e.g., an SV40i intron, a MVM intron, a Mv2 intron, an HNRNPH1 intron, chimeric introns or synthetic introns).

In certain embodiments, the bidirectional promoter does not comprise a viral promoter and/or a synthetic promoter. In certain embodiments, the compact promoter does not comprise F5tg83.

In certain embodiments, the bidirectional promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a bidirectional promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### III. Genes, Expression Constructs, and Expression Vectors

A coding sequence of a gene (e.g. CFTR, ATP7B, ATP7A, AGL, DMD and CPS1), or a functional fragment or variant thereof, may be provided in an expression construct and the construct itself may be provided as a transgene in the recombinant AAV (rAAV) vectors of the disclosure. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. In certain embodiments, the transgene is derived from a human.

In certain embodiments, the coding sequence is expressed in a target cell. In certain embodiments, the target cell is a lung cell, a pancreatic cell, a liver cell, or a neuronal cell.

In certain embodiments, the coding sequence is between about 4110 bp and about 6000 bp. In certain embodiments, the coding sequence is between about 4110 bp and about 5000 bp. In certain embodiments, the coding sequence is between about 4110 bp and about 5500 bp. In certain embodiments, the coding sequence is between about 4200 bp and about 5000 bp. In certain embodiments, the coding sequence is between about 4200 bp and about 5500 bp. In certain embodiments, the coding sequence is between about 4200 bp and about 6000 bp. In certain embodiments, the coding sequence is between about 4300 bp and about 5000 bp. In certain embodiments, the coding sequence is between about 4300 bp and about 5500 bp. In certain embodiments, the coding sequence is between about 4300 bp and about 6000 bp. In certain embodiments, the coding sequence is between about 4500 bp and about 5000 bp. In certain embodiments, the coding sequence is between about 4500 bp and about 5500 bp. In certain embodiments, the coding sequence is between about 4500 bp and about 6000 bp. In certain embodiments, the coding sequence is between about 4600 bp and about 5000 bp. In certain embodiments, the coding sequence is between about 4600 bp and about 5500 bp. In certain embodiments, the coding sequence is between about 4600 bp and about 6000 bp. In certain embodiments, the coding sequence is between about 4700 bp and about 5000 bp. In certain embodiments, the coding sequence is between about 4700 bp and about 5500 bp. In certain embodiments, the coding sequence is between about 4700 bp and about 6000 bp.

In some embodiments, in addition to a large gene or a functional fragment or variant thereof, the rAAV vector may also encode additional proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated subject. The additional proteins, peptides, RNA, enzymes, or catalytic RNAs and the complement factor may be encoded by a single vector carrying two or more heterologous sequences, or using two or more rAAV vectors each carrying one or more heterologous sequences.

### Cystic Fibrosis Transmembrane Conductance Regulator (CFTR)

In certain aspects, the disclosure provides nucleic acid comprising a coding sequence of a gene (e.g., a transgene, optionally in a recombinant adeno-associated viral (rAAV) vector) wherein the coding sequence encodes a human Cystic Fibrosis Transmembrane Conductance Regulator (CFTR) protein or biologically active fragment thereof. In certain embodiments, the coding sequence comprises a nucleotide sequence that is at least 80%, 85%, 90%, 92%, 94%, 95%, 97%, 99% or 100% identical to any of the sequences disclosed herein encoding a CFTR protein, or biologically active fragments thereof. In certain embodiments, the coding sequence comprises a nucleotide sequence that is at least 80%, 85%, 90%, 92%, 94%, 95%, 97%, 99% or 100% identical to any of SEQ ID NOs: 1-14, or biologically active fragments thereof. In certain embodiments, the coding sequence comprises a nucleotide sequence that encodes SEQ ID NO: 926, a sequence that is at least 80%, 85%, 90%, 92%, 94%, 95%, 97%, or 99% identical thereto, or biologically active fragments of any of the foregoing.

In certain aspects, the disclosure relates to a codon-optimized CFTR coding sequence. The codon-optimized CFTR coding sequence can include one or more of the following features as compared to a wild type CFTR coding sequence: (a) fewer unpaired base pairs of mRNA; (b) increased codon usage bias; (c) decreased GC content; (d) fewer CpG dinucleotides; (e) increased mRNA secondary structure; (f) fewer cryptic splicing sites; (g) fewer premature poly(A) sites; (h) fewer RNA instability motifs; (i) fewer AT-rich elements (ARE); (j) fewer repeat sequences (e.g., direct repeat, reverse repeat, and dyad repeat); (k) fewer GC peaks; and (1) fewer cis-acting elements. Accordingly, in certain embodiments, an optimized CFTR coding sequence comprises at least 1, at least 2, at least 3, at least 4, at least 5, at least 6, at least 7, at least 8, or at least 9 fewer unpaired base pairs of mRNA, CpG dinucleotides, cryptic splicing sites, premature poly(A) sites, RNA instability motifs; AT-rich elements (ARE), repeat sequences (e.g., direct repeat, reverse repeat, and dyad repeat), GC peaks, and cis-acting elements.

In certain embodiments, base-pairing among the first 10 residues of an optimized CFTR coding sequence will be minimized, because such base-pairing is known to affect translation. In certain embodiments, the first 10 residues of an optimized CFTR coding sequence, when tested (e.g., computationally tested) for secondary structure, will exhibit at least 1, at least 2, at least 3, at least 4, at least 5, at least 6, at least 7, at least 8, or at least 9 fewer base-pairings (e.g., base pairings with another nucleotide in the optimized CFTR) as compared to a wild-type CFTR coding sequence.

In certain embodiments, model unstructured UTRs (such as human β-globin 5′UTR and rabbit β-globin 3′UTR) can be added to flank a CFTR coding sequence and compared to an optimized CFTR sequence having the model unstructured UTRs and computationally refolded to confirm the absence of extensive base-pairing occurring between each optimized sequence and the model UTRs. Computational folding programs are known in the art, including, but not limited to, RNAfold (available at URL: rna.tbi.univie.ac.at/cgi-bin/RNAWebSuite/RNAfold.cgi), RNAstructure (available at URL: ma.urmc.rochester.edu/RNAstructureWeb/Servers/Predict1/Predict1.html); CONTRAfold (available at URL: contra.stanford.edu/contrafold/server.html); Mfold (available at URL: unafold.rna.albany.edu/?q=mfold); CentroidFold (available at URL: www.ncma.org/softwares/); LinearFold (available at URL: linearfold.org/). In certain embodiments, the minimum free energy structure of the nucleic acid comprising the 3′UTR, the 5′UTR or the 3′UTR and the 5′UTR does not favor base-pairing between (a) the 3′UTR, the 5′UTR or the 3′UTR and the 5′UTR and (b) the CFTR coding sequence.

A CFTR coding sequence can be optimized using the codon adaptive index (CAI). In certain embodiments, an optimized CFTR coding sequence has a CAI score of greater than 0.70, for example, between about 0.70 and about 0.90. In certain embodiments, an optimized CFTR coding sequence has a frequency of optimal codons (FOP) of greater than 80%, for example, from about 80% to about 90%. In certain embodiments, an optimized CFTR coding sequence has a GC content of between about 30-70%, for example, from about 30% to about 40%, about 30% to about 50%, from about 30% to about 60%, from about 40% to about 50%, from about 40% to about 60%, or from about 40% to about 70%. Unfavorable GC peaks are optimized to prolong the half-life of the mRNA.

A CFTR coding sequence can be optimized by removing cis-acting elements, including splice donors/acceptors (GGTAAG, GGTGAT, GTAAAA, GTAAGT), PolyA (AATAAA, ATTAAA, AAAAAAA), destabilizing motifs (ATTTA), AT-rich elements (ATTTTA, ATTTTTA, ATTTTTTA), PolyT (TTTTTT), polymerase slippage sites (GGGGGG, CCCCCC), and internal Kozak sequences (ACCACCATGG, GCCACCATGG). In certain embodiments, an optimized CFTR coding sequence comprises at least 1, at least 2, at least 3, at least 4, at least 5, at least 6, at least 7, at least 8, or at least 9 fewer cis-acting elements as compared to a wild-type CFTR coding sequence.

Stem loop structures and antiviral motifs (TGTGT, AACGTT, CGTTCG, AGCGCT, GACGTC, GACGTT) can interfere with ribosomal biding and mRNA stability. Accordingly, in certain embodiments, a CFTR coding sequence comprises at least 1, at least 2, at least 3, at least 4, at least 5, at least 6, at least 7, at least 8, or at least 9 fewer stem loop structures or antiviral motifs as compared to a wild-type CFTR coding sequence.

In certain embodiments, the calculated free energy (ΔG) of the codon optimized CFTR coding sequence is less than that of a wild-type CFTR coding sequence. In certain embodiments, the calculated free energy (ΔG) of the codon optimized CFTR coding sequence is about 300 kcal/mol to about 2000 kcal/mol less than that of a wild-type CFTR coding sequence.

In certain embodiments, the codon optimized CFTR coding sequence comprises fewer unpaired bases than a corresponding wild-type CFTR sequence. In certain embodiments, the codon optimized CFTR coding sequence comprises between about 15% and about 30% unpaired bases, between about 15% and about 25%, between about 17% and about 22% unpaired bases as predicted by LinearFold (http://linearfold.org/). In certain embodiments, the codon optimized CFTR coding sequence comprises between about 45% and about 85% the number of unpaired bases of a corresponding wild-type CFTR sequence, for example, from about 45% to about 80%, about 45% to about 75%, about 45% to about 70%, about 45% to about 65%, about 45% to about 60%, about 45% to about 55%, about 45% to about 50%, about 50% to about 85%, about 50% to about 80%, about 50% to about 75%, about 50% to about 70%, about 50% to about 65%, about 50% to about 60%, about 50% to about 55%, about 55% to about 85%, about 55% to about 80%, about 55% to about 75%, about 55% to about 70%, about 55% to about 65%, about 55% to about 60%, about 60% to about 85%, about 60% to about 80%, about 60% to about 75%, about 60% to about 70%, about 60% to about 65%, about 65% to about 85%, about 65% to about 80%, about 65% to about 75%, about 65% to about 70%, about 70% to about 85%, about 70% to about 80%, about 70% to about 75%, or about 75% to about 80 the number of unpaired bases of a corresponding wild-type CFTR sequence as predicted by an RNA folding prediction program (e.g., RNAfold, RNAstructure, CONTRAfold, Mfold, CentroidFold and LinearFold as described herein).

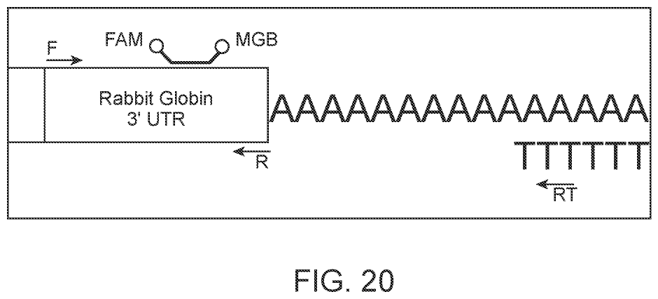

In order to assess the effects of optimization on mRNA stability, the half-life of a CFTR coding sequence can be determined by RT-qPCR following actinomycin treatment. The assay can be performed in A549 cells, which do not endogenously express CFTR. A primer pair and probes against the rabbit β-globin 3′UTR can be used to standardize detection between all constructs (see, e.g., FIG. 20). RT priming occurs via an oligo(dT) primer, and qPCR detection in the 3′UTR region can avoid potential issues arising from extensive RNA secondary structures within the coding sequences. Cells can be cultured and transfected with plasmids encoding the optimized CFTR coding sequences. Cells can be treated with a transcription arresting agent such as actinomycin D or DMSO at varying time points to arrest transcription. At each time point, cells are lysed, RNA is quantitated, and cDNA is synthesized. Expression and stability of each variant can be compared to the wild-type CFTR coding sequence. In addition, the effects of optimization on protein levels can be assessed by performing Western Blots on the transformed cells using antibodies against CFTR (e.g., ab596, CFF Antibody Distribution Program) and β-actin. In addition, the effects of codon optimization on AAV packaging can be assessed by small-scale packaging experiments. Detailed protocols for these assessments are found in the Examples.

In certain aspects, the disclosure provides a nucleic acid comprising a codon-optimized CFTR coding sequence or biologically active fragment thereof. In certain embodiments, the coding sequence comprises a nucleotide sequence of SEQ ID NOs: 3-14. In certain embodiments, the coding sequence comprises a nucleotide sequence that is at least 80%, 85%, 90%, 92%, 94%, 95%, 97%, 99% or 100% identical to any of SEQ ID NOs: 3-14, or biologically active fragments thereof. In certain embodiments, the coding sequence comprises a nucleotide sequence that encodes SEQ ID NO: 926, a sequence that is at least 80%, 85%, 90%, 92%, 94%, 95%, 97%, or 99% identical thereto, or biologically active fragments of any of the foregoing.

### Copper-Transporting P-Type ATPase (ATP7B and ATP7A)

In certain aspects, the disclosure provides a nucleic acid comprising a coding sequence of a gene (e.g., a transgene, optionally in a recombinant adeno-associated viral (rAAV) vector), wherein the coding sequence encodes a human copper-transporting P-type ATPase protein or biologically active fragment thereof. In certain embodiments, the coding sequence comprises a nucleotide sequence that is at least 80%, 85%, 90%, 92%, 94%, 95%, 97%, 99% or 100% identical to any of the sequences disclosed herein encoding a ATP7B protein, or biologically active fragments thereof. In certain embodiments, the coding sequence comprises a nucleotide sequence that is at least 80%, 85%, 90%, 92%, 94%, 95%, 97%, 99% or 100% identical to any of SEQ ID NO: 15, or biologically active fragments thereof. In certain embodiments, the coding sequence comprises a nucleotide sequence that encodes SEQ ID NO: 927, a sequence that is at least 80%, 85%, 90%, 92%, 94%, 95%, 97%, or 99% identical thereto, or biologically active fragments of any of the foregoing.

In certain aspects, the disclosure provides a nucleic acid comprising a coding sequence of a gene (e.g., a transgene, optionally in a recombinant adeno-associated viral (rAAV) vector) wherein the coding sequence encodes a human copper-transporting P-type ATPase protein or biologically active fragment thereof. In certain embodiments, the coding sequence comprises a nucleotide sequence that is at least 80%, 85%, 90%, 92%, 94%, 95%, 97%, 99% or 100% identical to any of the sequences disclosed herein encoding a ATP7A protein, or biologically active fragments thereof. In certain embodiments, the coding sequence comprises a nucleotide sequence that is at least 80%, 85%, 90%, 92%, 94%, 95%, 97%, 99% or 100% identical to any of SEQ ID NO: 16, or biologically active fragments thereof. In certain embodiments, the coding sequence comprises a nucleotide sequence that encodes SEQ ID NO: 928, a sequence that is at least 80%, 85%, 90%, 92%, 94%, 95%, 97%, or 99% identical thereto, or biologically active fragments of any of the foregoing.

### Amylo-Alpha-1, 6-Glucosidase, 4-Alpha-Glucanotransferase (AGL)

In certain aspects, the disclosure provides a nucleic acid comprising a coding sequence of a gene (e.g., a transgene, optionally in a recombinant adeno-associated viral (rAAV) vector) wherein the coding sequence encodes a human Amylo-Alpha-1, 6-Glucosidase, 4-Alpha-Glucanotransferase (AGL) protein or biologically active fragment thereof. In certain embodiments, the coding sequence comprises a nucleotide sequence that is at least 80%, 85%, 90%, 92%, 94%, 95%, 97%, 99% or 100% identical to any of the sequences disclosed herein encoding a AGL protein, or biologically active fragments thereof. In certain embodiments, the coding sequence comprises a nucleotide sequence that is at least 80%, 85%, 90%, 92%, 94%, 95%, 97%, 99% or 100% identical to any of SEQ ID NO: 17, or biologically active fragments thereof. In certain embodiments, the coding sequence comprises a nucleotide sequence that encodes SEQ ID NO: 929, a sequence that is at least 80%, 85%, 90%, 92%, 94%, 95%, 97%, or 99% identical thereto, or biologically active fragments of any of the foregoing.

### Duchenne Muscular Dystrophy (DMD)

In certain aspects, the disclosure provides a nucleic acid comprising a coding sequence of a gene (e.g., a transgene, optionally in a recombinant adeno-associated viral (rAAV) vector) wherein the coding sequence encodes a human dystrophin (DMD) protein or biologically active fragment thereof. Exemplary biologically active fragments of DMD include minidystrophin and microdystrophin, and those biologically active fragments disclosed in U.S. Pat. No. 10,351,611. In certain embodiments, the coding sequence comprises a nucleotide sequence that is at least 80%, 85%, 90%, 92%, 94%, 95%, 97%, 99% or 100% identical to any of the sequences disclosed herein encoding a DMD protein, or biologically active fragments thereof. In certain embodiments, the coding sequence comprises a nucleotide sequence that is at least 80%, 85%, 90%, 92%, 94%, 95%, 97%, 99% or 100% identical to SEQ ID NO: 18, or biologically active fragments thereof.

### Carbamoyl Phosphate Synthetase I (CPS1)

In certain aspects, the disclosure provides a nucleic acid (e.g., a transgene, optionally in a recombinant adeno-associated viral (rAAV) vector) encoding a human carbamoyl phosphate synthetase I (CPS1) protein or biologically active fragment thereof. In certain embodiments, the nucleic acid sequence is at least 80%, 85%, 90%, 92%, 94%, 95%, 97%, 99% or 100% identical to any of the sequences disclosed herein encoding a CPS1 protein, or biologically active fragments thereof. In certain embodiments, the nucleic acid sequence is at least 80%, 85%, 90%, 92%, 94%, 95%, 97%, 99% or 100% identical to any of SEQ ID NO: 19-24, or biologically active fragments thereof. In certain embodiments, the coding sequence comprises a nucleotide sequence that encodes any one of SEQ ID NOs: 930-935, a sequence that is at least 80%, 85%, 90%, 92%, 94%, 95%, 97%, or 99% identical thereto, or biologically active fragments of any of the foregoing.

In one aspect, a transgene comprises a large gene or a functional fragment or variant thereof that encodes a polypeptide with 1, 2, 3, 4, 5, 6, 7, 8, 9, 10, 11, 12, 13, 14, or 15 amino acid substitutions, deletions, and/or additions relative to the wild-type polypeptide. In some embodiments, a transgene encodes a complement system polypeptide with 1, 2, 3, 4, or 5 amino acid deletions relative to the wild-type polypeptide. In some embodiments, a transgene encodes a polypeptide with 1, 2, 3, 4, or 5 amino acid substitutions relative to the wild-type polypeptide. In some embodiments, a transgene encodes a polypeptide with 1, 2, 3, 4, or 5 amino acid insertions relative to the wild-type polypeptide. Polynucleotides complementary to any of the polynucleotide sequences disclosed herein are also encompassed by the present disclosure. Polynucleotides may be single-stranded (coding or antisense) or double-stranded, and may be DNA (genomic or synthetic), cDNA, or RNA molecules. RNA molecules include mRNA molecules. Additional coding or non-coding sequences may, but need not, be present within a polynucleotide of the present disclosure, and a polynucleotide may, but need not, be linked to other molecules and/or support materials.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins et al. (1989) CABIOS 5: 151-153; Myers et al. (1988) CABIOS 4: 11-17; Robinson, E. D., 1971, Comb. Theor. 11: 105; Santou et al (1987), MOL. BIOL. EVOL. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman (1983) PROC. NATL. ACAD. SCI. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. The transgenes or variants may also, or alternatively, be substantially homologous to a native gene, or a portion or complement thereof. Such polynucleotide variants are capable of hybridizing under moderately stringent conditions to a naturally occurring DNA sequence encoding a complement factor (or a complementary sequence). Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS. As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in SEQ ID NOs: 1-24, or sequences complementary thereto. One of ordinary skill in the art will readily understand that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

The present disclosure further provides oligonucleotides that hybridize to a polynucleotide having the nucleotide sequence set forth in SEQ ID NOs: 1-24, or to a polynucleotide molecule having a nucleotide sequence which is the complement of a sequence listed above. Such oligonucleotides are at least about 10 nucleotides in length, and preferably from about 15 to about 30 nucleotides in length, and hybridize to one of the aforementioned polynucleotide molecules under highly stringent conditions, i.e., washing in 6>SSC/0.5% sodium pyrophosphate at about 37° C. for about 14-base oligos, at about 48° C. for about 17-base oligos, at about 55° C. for about 20-base oligos, and at about 60° C. for about 23-base oligos. In a preferred embodiment, the oligonucleotides are complementary to a portion of one of the aforementioned polynucleotide molecules. These oligonucleotides are useful for a variety of purposes including encoding or acting as antisense molecules useful in gene regulation, or as primers in amplification of complement system-encoding polynucleotide molecules.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

The large gene (e.g. CFTR, ATP7B, ATP7A, AGL, DMD, and CPS1) or a functional fragment or variant thereof may be used to correct or ameliorate gene deficiencies, which may include deficiencies in which normal large genes are expressed at less than normal levels or deficiencies in which the functional complement system gene product is not expressed. In some embodiments, the transgene sequence encodes a single large gene or a functional fragment or variant thereof. The disclosure further includes using multiple transgenes, e.g., transgenes encoding two or more large genes (e.g. CFTR, ATP7B, ATP7A, AGL, DMD, and CPS1), or a functional fragments or variants thereof. In certain situations, a different transgene may be used to encode different large genes or a functional fragments or variants thereof (e.g. CFTR, ATP7B, ATP7A, AGL, DMD, and CPS1). Alternatively, different large genes (e.g. CFTR, ATP7B, ATP7A, AGL, DMD, and CPS1) or functional fragments or variants thereof, may be encoded by the same transgene.

The regulatory sequences include conventional control elements which are operably linked to the transgene comprising a large gene (e.g. CFTR, ATP7B, ATP7A, AGL, DMD, and CPS1), or a functional fragment or variant thereof, in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (poly A) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. In some embodiments, the intron comprises the nucleotide sequence of SEQ ID NO: 10, or a codon-optimized or fragment thereof. Another suitable sequence includes the woodchuck hepatitis virus post-transcriptional element. (See, e.g., L. Wang and I. Verma, 1999 PROC. NATL. ACAD. SCI., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. Preferably, the IRES is located 3′ to the transgene in the rAAV vector.

Other regulatory sequences useful herein include enhancer sequences. Enhancer sequences useful herein include the IRBP enhancer, immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al., and references cited therein at, for example, pages 3.18-3.26 and 16.17-16.27 and Ausubel et al., Current Protocols in Molecular Biology, John Wiley & Sons, New York, 1989). It is understood that not all vectors and expression control sequences will function equally well to express all of the transgenes as described herein. However, one of skill in the art may make a selection among these, and other, expression control sequences to generate the rAAV vectors of the disclosure.

In certain embodiments, the expression construct includes a coding sequence (e.g., a protein coding sequence). In certain embodiments, the expression construct is present in an rAAV vector to target a specific cell type. In certain embodiments, the expression construct is expressed in a target cell (e.g., a lung cell, a pancreatic cell, a liver cell, an epithelial cell, or a neuronal cell). In certain embodiments, the expression construct is expressed in a Calu-3 cells, CFBE4lo− cells, or in A549 cells. In certain embodiments, the expression construct is expressed in HEK293 cells. In certain embodiments, the expression construct is expressed in HeLa cells.

In certain embodiments, the expression construct includes the coding sequence of a large gene (e.g., CFTR, ATP7B, ATP7A, AGL, DMD, and CPS1). In certain embodiments, expressing CFTR protein in an epithelial cell causes an increase in transepithelial electrical resistance (TEER) as compared to a cell in which the expression construct is not present and/or expressed. In certain embodiments, expressing CFTR protein in an epithelial cell causes an increase in transepithelial Cl⁻ transport as compared to a cell in which the expression construct is not present and/or expressed.

### IV. Nucleic Acids with a Bidirectional Promoter

In certain embodiments, the expression construct includes a compact bidirectional promoter, a protein coding gene, and, optionally, a second gene comprising a second coding sequence that encodes an RNA molecule or a second protein. In certain embodiments, the compact bidirectional promoter has a size between 50 bp and 250 bp (e.g., from 60 bp to 240 bp, from 70 bp to 230 bp, from 80 bp to 220 bp, from 90 bp to 210 bp, from 100 bp to 200 bp, from 110 bp to 190 bp, from 120 bp to 180 bp, from 130 bp to 170 bp, or from 140 bp to 160 bp). In some embodiments, the compact bidirectional promoter has a size between 50 bp and 200 bp (e.g., from 60 bp to 190 bp, from 70 bp to 180 bp, from 80 bp to 170 bp, from 90 bp to 160 bp, from 100 bp to 150 bp, from 110 bp to 140 bp, or from 120 bp to 130 bp). In some embodiments, the compact bidirectional promoter has a size between 50 bp and 180 bp (from 60 bp to 170 bp, from 70 bp to 160 bp, from 80 bp to 150 bp, from 90 bp to 140 bp, from 100 bp to 130 bp, from 110 bp to 120 bp).

In certain embodiments, the protein coding gene comprises a large gene as described herein.

In certain embodiments, the second gene encodes a molecule (e.g., an RNA molecule or a second protein) smaller than the protein encoded by the protein coding gene. In certain embodiments, the second coding sequence encodes a molecule (e.g., an RNA molecule or a second protein) larger than the protein encoded by the protein coding gene. In certain embodiments, the second coding sequence encodes a molecule (e.g., an RNA molecule or a second protein) having a substantially equal size to the protein encoded by the protein encoding gene.

In certain embodiments, the second gene has a coding sequence between about 300 bp and about 6000 bp. In certain embodiments, the coding sequence is between about 400 bp and about 5000 bp. In certain embodiments, the coding sequence is between about 500 bp and about 4000 bp. In certain embodiments, the coding sequence is between about 600 bp and about 3000 bp. In certain embodiments, the coding sequence is between about 700 bp and about 2000 bp. In certain embodiments, the coding sequence is between about 800 bp and about 2000 bp. In certain embodiments, the coding sequence is between about 900 bp and about 2000 bp. In certain embodiments, the coding sequence is between about 1000 bp and about 2000 bp. In certain embodiments, the coding sequence is between about 1100 bp and about 2000 bp. In certain embodiments, the coding sequence is between about 1200 bp and about 2000 bp. In certain embodiments, the coding sequence is between about 1300 bp and about 2000 bp. In certain embodiments, the coding sequence is between about 1400 bp and about 1900 bp. In certain embodiments, the coding sequence is between about 1400 bp and about 1800 bp. In certain embodiments, the coding sequence is between about 1400 bp and about 1700 bp. In certain embodiments, the coding sequence is between about 1400 bp and about 1500 bp.

In some embodiments, the compact bidirectional promoter is an H1 promoter. In some embodiments, the H1 promoter is a human H1 promoter. In certain embodiments, the nucleic acid comprises a compact bidirectional promoter and a coding sequence that encodes a cystic fibrosis transmembrane conductance regulator (CFTR), ATP7B, ATP7A, AGL, CPS1, or a functional fragment or variant thereof. In certain embodiments, the nucleic acid having a compact bidirectional promoter has a coding sequence encoding CFTR as described herein. In certain embodiments, an expression construct having a compact bidirectional promoter can be expressed in any target cell described herein. In certain embodiments, the invention herein provides a vector including the expression construct encoding the compact bidirectional promoter as described herein. Furthermore, in some embodiments, the vector is any of the herein described AAV vectors.

### V. Construction of rAAV Vectors

The disclosure provides recombinant AAV (rAAV) vectors comprising a large gene (e.g., CFTR, ATP7B, ATP7A, AGL, DMD, and CPS1), or a functional fragment or variant thereof, under the control of a suitable promoter (e.g., a compact promoter) to direct the expression of the large gene, or functional fragment or variant thereof in a target cell. In certain embodiments, the rAAV vectors include the coding sequence of at least one of the large genes herein described (e.g., CFTR, ATP7B, ATP7A, AGL, DMD, and CPS1). In certain embodiments, the coding sequence is expressed in a target cell. In certain embodiments, the target cell is a lung cell, a pancreatic cell, a liver cell, or a neuronal cell. The disclosure further provides a therapeutic composition comprising an rAAV vector comprising a large gene (e.g. CFTR, ATP7B, ATP7A, AGL, DMD, and CPS1), or a functional fragment or variant thereof under the control of a suitable promoter (e.g., a compact promoter). A variety of rAAV vectors may be used to deliver the desired complement system gene to the appropriate cells and/or tissues and to direct its expression. More than 30 naturally occurring serotypes of AAV from humans and non-human primates are known. Many natural variants of the AAV capsid exist, and an rAAV vector of the disclosure may be designed based on an AAV with properties specifically suited for expression in the cells and/or tissues relevant for the large gene (e.g. CFTR, ATP7B, ATP7A, AGL, DMD, and CPS1) to be expressed.

In general, an rAAV vector is comprised of, in order, a 5′ adeno-associated virus inverted terminal repeat, a transgene or gene of interest encoding a complement system polypeptide (e.g. CFTR, ATP7B, ATP7A, AGL, DMD, and CPS1) or a functional fragment or variant thereof operably linked to a sequence which regulates its expression in a target cell, and a 3′ adeno-associated virus inverted terminal repeat. In addition, the rAAV vector may preferably have a polyadenylation sequence. Generally, rAAV vectors should have one copy of the AAV ITR at each end of the transgene or gene of interest, in order to allow replication, packaging, and efficient integration into cell chromosomes. Within preferred embodiments of the disclosure, the transgene sequence encoding a complement system polypeptide (or a functional fragment or variant thereof) or a biologically active fragment thereof will be of about 2 to 5 kb in length (or alternatively, the transgene may additionally contain a “stuffer” or “filler” sequence to bring the total size of the nucleic acid sequence between the two ITRs to between 2 and 5 kb).

Recombinant AAV vectors of the present disclosure may be generated from a variety of adeno-associated viruses to provide the rAAV with cell-type-specific targeting capacity or tropism. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV 1 through AAV12. The tropism of the vector may be altered by packaging the recombinant genome of one serotype into capsids derived from another AAV serotype. In some embodiments, the ITRs of the rAAV virus may be based on the ITRs of any one of AAV 1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure.

Examples of AAV serotypes include AAV1, AAV2, AAV3, AAV4, AAV5, AAV6, AAV7, AAV8, AAV9, AAV10, AAV11, AAV 12, AAV-DJ, AAV-DJ8, AAV-DJ9, AAVrh8, AAVrh8R or AAVrh10. In certain embodiments, the AAV capsid serotype is AAV2.

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid.

Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof.

Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV 12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the large gene or a functional fragment or variant thereof to the nucleus. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), lie (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gln; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a R-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxy lysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. VIROL., 70:520-532 (1993) and U.S. Pat. No. 5,478,745. These publications are incorporated by reference herein.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). In some embodiments, vectors suitable for use with the present disclosure may be pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells may also be transfected with a vector (e.g., helper vector) which provides helper functions to the AAV. The vector providing helper functions may provide adenovirus functions, including, e.g., E1a, E1b, E2a, E40RF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

An rAAV vector of the disclosure is generated by introducing a nucleic acid sequence encoding an AAV capsid protein, or fragment thereof; a functional rep gene or a fragment thereof; a minigene composed of, at a minimum, AAV inverted terminal repeats (ITRs) and a transgene; and sufficient helper functions to permit packaging of the minigene into the AAV capsid, into a host cell. The components required for packaging an AAV minigene into an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In some embodiments, such a stable host cell will contain the required component(s) under the control of an inducible promoter. Alternatively, the required component(s) may be under the control of a constitutive promoter. Examples of suitable inducible and constitutive promoters are provided herein, in the discussion below of regulator elements suitable for use with the transgene, i.e., a nucleic acid comprising a large gene or a functional fragment or variant thereof (e.g. CFTR, ATP7B, ATP7A, AGL, DMD, and CPS1). In still another alternative, a selected stable host cell may contain selected components under the control of a constitutive promoter and other selected components under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the disclosure may be delivered to the packaging host cell in the form of any genetic element which transfers the sequences. The selected genetic element may be delivered by any suitable method known in the art. See, e.g., Sambrook et al., Molecular Cloning: A Laboratory Manual, Cold Spring Harbor Press, Cold Spring Harbor, NY.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV10, AAV11, AAV 12, AAV-DJ, AAV-DJ8, AAV-DJ9, AAVrh8, AAVrh8R or AAVrh10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

The minigene is composed of, at a minimum, a transgene comprising a large gene or a functional fragment or variant thereof (e.g. CFTR, ATP7B, ATP7A, AGL, DMD, and CPS1), as described above, and its regulatory sequences, and 5′ and 3′ AAV inverted terminal repeats (ITRs). In one desirable embodiment, the ITRs of AAV serotype 2 are used. However, ITRs from other suitable serotypes may be selected. The minigene is packaged into a capsid protein and delivered to a selected host cell.

In some embodiments, regulatory sequences are operably linked to the transgene comprising a large gene or a functional fragment or variant thereof (e.g. CFTR, ATP7B, ATP7A, AGL, DMD, and CPS1). The regulatory sequences may include conventional control elements which are operably linked to the complement system gene, splice variant, or a fragment thereof in a manner which permits its transcription, translation and/or expression in a cell transfected with the vector or infected with the virus produced by the disclosure.

The regulatory sequences useful in the constructs of the present disclosure may also contain an intron, desirably located between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 PROC. NATL. ACAD. SCI., USA, 96:3906-3910). PolyA signals may be derived from many suitable species, including, without limitation SV-40, human and bovine.

Another regulatory component of the rAAV useful in the method of the disclosure is an internal ribosome entry site (IRES). An IRES sequence, or other suitable systems, may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In some embodiments, expression of the transgene comprising a large gene or a functional fragment or variant thereof (e.g. CFTR, ATP7B, ATP7A, AGL, DMD, and CPS1) is driven by a separate promoter (e.g., a viral promoter). In certain embodiments, any promoters suitable for use in AAV vectors may be used with the vectors of the disclosure. The selection of the transgene promoter to be employed in the rAAV may be made from among a wide number of constitutive or inducible promoters that can express the selected transgene in the desired cell. Examples of suitable promoters are described in detail below.

Other regulatory sequences useful in the disclosure include enhancer sequences. Enhancer sequences useful in the disclosure include the 1RBP enhancer, immediate early cytomegalovirus enhancer, one derived from an immunoglobulin gene or SV40 enhancer, the cis-acting element identified in the mouse proximal promoter, etc.

Selection of these and other common vector and regulatory elements are well-known and many such sequences are available. See, e.g., Sambrook et al., and references cited therein at, for example, pages 3.18-3.26 and 16, 17-16.27 and Ausubel et al., Current Protocols in Molecular Biology, John Wiley & Sons, New York, 1989).

The rAAV vector may also contain additional sequences, for example from an adenovirus, which assist in effecting a desired function for the vector. Such sequences include, for example, those which assist in packaging the rAAV vector in adenovirus-associated virus particles.

The rAAV vector may also contain a reporter sequence for co-expression, such as but not limited to lacZ, GFP, CFP, YFP, RFP, mCherry, tdTomato, etc. In some embodiments, the rAAV vector may comprise a selectable marker. In some embodiments, the selectable marker is an antibiotic-resistance gene. In some embodiments, the antibiotic-resistance gene is an ampicillin-resistance gene. In some embodiments, the ampicillin-resistance gene is beta-lactamase.

In some embodiments, the rAAV particle is an ssAAV. In some embodiments, the rAAV particle is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to −55 kd) and any currently available RNA-based therapy.

The single-stranded nature of the AAV genome may impact the expression of rAAV vectors more than any other biological feature. Rather than rely on potentially variable cellular mechanisms to provide a complementary-strand for rAAV vectors, it has now been found that this problem may be circumvented by packaging both strands as a single DNA molecule. In the studies described herein, an increased efficiency of transduction from duplexed vectors over conventional rAAV was observed in He La cells (5-140 fold). More importantly, unlike conventional single-stranded AAV vectors, inhibitors of DNA replication did not affect transduction from the duplexed vectors of the invention. In addition, the inventive duplexed parvovirus vectors displayed a more rapid onset and a higher level of transgene expression than did rAAV vectors in mouse hepatocytes in vivo. All of these biological attributes support the generation and characterization of a new class of parvovirus vectors (delivering duplex DNA) that significantly contribute to the ongoing development of parvovirus-based gene delivery systems.

Overall, a novel type of parvovirus vector that carries a duplexed genome, which results in co-packaging strands of plus and minus polarity tethered together in a single molecule, has been constructed and characterized by the investigations described herein. Accordingly, the present invention provides a parvovirus particle comprising a parvovirus capsid (e.g., an AAV capsid) and a vector genome encoding a heterologous nucleotide sequence, where the vector genome is self-complementary, i.e., the vector genome is a dimeric inverted repeat. The vector genome is preferably approximately the size of the wild-type parvovirus genome (e.g., the AAV genome) corresponding to the parvovirus capsid into which it will be packaged and comprises an appropriate packaging signal. The present invention further provides the vector genome described above and templates that encode the same.

rAAV vectors useful in the methods of the disclosure are further described in PCT publication No. WO2015168666 and PCT publication no. WO2014011210, the contents of which are incorporated by reference herein.

In some embodiments, any of the vectors disclosed herein is capable of inducing at least 20%, 50%, 100%, 150%, 200%, 250%, 300%, 400%, 500%, 700%, 900%, 1000%, 1100%, 1500%, or 2000% higher expression of CFTR, ATP7B, ATP7A, AGL, DMD, or CPS1 in a target cell as compared to the endogenous expression of CFTR, ATP7B, ATP7A, AGL, DMD, or CPS1 in the target cell. In some embodiments, expression of any of the vectors disclosed herein in a target cell results in at least 20%, 50%, 100%, 150%, 200%, 250%, 300%, 400%, 500%, 700%, 900%, 1000%, 1100%, 1500%, or 2000% higher levels of CFTR, ATP7B, ATP7A, AGL, DMD, or CPS1 activity in the target cell as compared to endogenous levels of CFTR, ATP7B, ATP7A, AGL, DMD, or CPS1 activity in the target cell.

### VI. Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997). Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a transgene comprising a large gene (e.g. CFTR, ATP7B, ATP7A, AGL, DMD, and CPS1), or a functional fragment or variant thereof) flanked by at least one AAV ITR sequence; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The rAAV particles can be produced using methods known in the art. See, e.g., U.S. Pat. Nos. 6,566,118; 6,989,264; and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Recombinant AAV particles are generated by transfecting producer cells with a plasmid (cis-plasmid) containing a rAAV genome comprising a transgene flanked by the 145 nucleotide-long AAV ITRs and a separate construct expressing the AAV rep and CAP genes in trans. In addition, adenovirus helper factors such as ETA, E1B, E2A, E40RF6 and VA RNAs, etc. may be provided by either adenovirus infection or by transfecting a third plasmid providing adenovirus helper genes into the producer cells. Producer cells may be HEK293 cells. Packaging cell lines suitable for producing adeno-associated viral vectors may be readily accomplished given readily available techniques (see e.g., U.S. Pat. No. 5,872,005). The helper factors provided will vary depending on the producer cells used and whether the producer cells already carry some of these helper factors.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified.

In some embodiments, rAAV particles may be produced by a producer cell line method, such as the exemplary producer cell line method provided infra (see also (referenced in Martin et al., (2013) HUMAN GENE THERAPY METHODS 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a therapeutic polypeptide and/or nucleic acid as described herein flanked by at least one AAV ITR, and (iii) an AAV helper function; and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV 12, AAV2R471A, AAV DJ, a goat AAV, bovine AAV, or mouse AAV or the like. In some embodiments, the encapsidation protein is an AAV2 encapsidation protein.

Suitable rAAV production culture media of the present disclosure may be supplemented with serum or serum-derived recombinant proteins at a level of 0.5-20 (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2 pvi Filter Opticap XL 10 Millipore Express SHC Hydrophilic Membrane filter. Clarification can also be achieved by a variety of other standard techniques known in the art, such as, centrifugation or filtration through any cellulose acetate filter of 0.2 pvil or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/ml of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) JOURNAL OF VIROLOGY 72:2224-2232; U.S. Pat. Nos. 6,989,264 and 8,137,948; and WO 2010/148143.

### VII. Pharmaceutical Compositions

Also provided herein are pharmaceutical compositions comprising a nucleic acid comprising a large gene (e.g. CFTR, ATP7B, ATP7A, AGL, DMD, and CPS1), or a functional fragment or variant thereof, and a pharmaceutically acceptable carrier. The pharmaceutical compositions may be suitable for any mode of administration described herein.

In some embodiments, the pharmaceutical compositions comprising a nucleic acid described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the nucleic acid comprising the desired large gene (e.g. CFTR, ATP7B, ATP7A, AGL, DMD, and CPS1), or a functional fragment or variant thereof and constitutive or tissue or cell-specific promoter for use in the target cells as detailed above is formulated into a pharmaceutical composition intended for oral, inhalation, intranasal, intratracheal, intravenous, intramuscular, subcutaneous, intradermal, and other parental routes of administration. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). Routes of administration may be combined, if desired.

The composition may be delivered in a volume of from about 0.1 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. An effective concentration of a recombinant adeno-associated virus carrying a nucleic acid sequence encoding the desired transgene under the control of the cell-specific promoter sequence desirably ranges from about 10⁷ and 10¹³ vector genomes per milliliter (vg/mL) (also called genome copies/mL (GC/mL)). The rAAV infectious units are measured as described in S. K. McLaughlin et al., 1988 J. Virol., 62: 1963, which is incorporated herein by reference.

Preferably, the concentration in the target tissue is from about 1.5×10⁹ vg/mL to about 1.5×10¹² vg/mL, and more preferably from about 1.5×10⁹ vg/mL to about 1.5×10¹¹ vg/mL. In certain preferred embodiments, the effective concentration is about 2.5×10¹⁰ vg to about 1.4×10¹¹. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. In one embodiment, the effective dosage (total genome copies delivered) is from about 10⁷ to 10¹³ vector genomes. It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

Pharmaceutical compositions useful in the methods of the disclosure are further described in PCT publication No. WO2015168666 and PCT publication no. WO2014011210, the contents of which are incorporated by reference herein.

### VIII. Methods of Treatment/Prophylaxis

Described herein are various methods of preventing, treating, arresting progression of or ameliorating disease and disorders as described herein. Generally, the methods include administering to a subject, e.g., a mammalian subject, in need thereof, an effective amount of a composition comprising a recombinant adeno-associated virus (AAV) described above, carrying a transgene large gene or a functional fragment or variant thereof under the control of regulatory sequences which express the product of the gene in target cells of a subject, and a pharmaceutically acceptable carrier. Any of the AAV described herein are useful in the methods described below.

In a certain aspect, the disclosure provides a method of treating a subject having a disease as described herein, comprising the step of administering to the subject a vector of the disclosure. In certain embodiments, the vector is administered at a dose between 2.5×10¹⁰ vg and 1.4×10¹¹ vg. In certain embodiments, the vectors are administered at a dose between 1.0×10¹¹ vg and 1.5×10¹³ vg. In certain embodiments, the vectors are administered at a dose between 1.0×10¹¹ vg and 1.5×10¹² vg. In certain embodiments, the vectors are administered at a dose of about 1.4×10¹². In certain embodiments, the vectors are administered at a dose of 1.4×10¹² vg. In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the vectors are administered by intravitreal injection in a solution of PBS with additional NaCl and pluronic.

In some embodiments, any of the vectors disclosed herein is capable of inducing at least 20%, 50%, 100%, 150%, 200%, 250%, 300%, 400%, 500%, 700%, 900%, 1000%, 1100%, 1500%, or 2000% higher expression of CFTR, ATP7B, ATP7A, AGL, DMD, or CPS1 in a target cell as compared to the endogenous expression of CFTR, ATP7B, ATP7A, AGL, DMD, or CPS1 in the target cell. In some embodiments, expression of any of the vectors disclosed herein in a target cell results in at least 20%, 50%, 100%, 150%, 200%, 250%, 300%, 400%, 500%, 700%, 900%, 1000%, 1100%, 1500%, or 2000% higher levels of CFTR, ATP7B, ATP7A, AGL, DMD, or CPS1 activity in the target cell as compared to endogenous levels of CFTR, ATP7B, ATP7A, AGL, DMD, or CPS1 activity in the target cell.

In some embodiments, any of the vectors disclosed herein is administered to cell(s) or tissue(s) in a test subject. In some embodiments, the cell(s) or tissue(s) in the test subject express less CFTR, ATP7B, ATP7A, AGL, DMD, or CPS1, or less functional CFTR, ATP7B, ATP7A, AGL, DMD, or CPS1, than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with a mutation in and/or inactivation of a CFTR, ATP7B, ATP7A, AGL, DMD, or CPS1 gene. In some embodiments, the reference control subject does not have cystic fibrosis, Wilson disease, Menkes disease, Cori Disease, Duchenne Muscular Dystrophy, or CPS1D. In some embodiments, a target cell or tissue in the test subject expresses at least 95%, 90%, 80%, 70%, 60%, 50%, 40%, 30%, 20%, 10%, 5%, or 1% less CFTR, ATP7B, ATP7A, AGL, DMD, or CPS1 or functional CFTR, ATP7B, ATP7A, AGL, DMD, or CPS1 as compared to the levels in the reference control subject or population of reference control subjects. In some embodiments, a target cell or tissue in the test subject expresses CFTR, ATP7B, ATP7A, AGL, DMD, or CPS1 protein having any of the mutations disclosed herein. In some embodiments, a target cell or tissue in the reference control subject does not express a CFTR, ATP7B, ATP7A, AGL, DMD, or CPS1 protein having any of the CFTR, ATP7B, ATP7A, AGL, DMD, or CPS1 mutations disclosed herein. In some embodiments, expression of any of the vectors disclosed herein in the cell(s) or tissue(s) of the test subject results in an increase in levels of CFTR, ATP7B, ATP7A, AGL, DMD, or CPS1 protein or functional CFTR, ATP7B, ATP7A, AGL, DMD, or CPS1 protein. In some embodiments, expression of any of the vectors disclosed herein in the cell(s) or tissue(s) of the test subject results in an increase in levels of CFTR, ATP7B, ATP7A, AGL, DMD, or CPS1 protein or functional CFTR, ATP7B, ATP7A, AGL, DMD, or CPS1 protein such that the increased levels are within 90%, 80%, 70%, 60%, 50%, 40%, 30%, 20%, 10%, 5%, or 1% of, or are the same as, the levels of CFTR, ATP7B, ATP7A, AGL, DMD, or CPS1 protein or functional CFTR, ATP7B, ATP7A, AGL, DMD, or CPS1 protein expressed by the same cell type or tissue type in the reference control subject or population of reference control subjects. In some embodiments, expression of any of the vectors disclosed herein in the cell(s) or tissue(s) of the test subject results in an increase in levels of CFTR, ATP7B, ATP7A, AGL, DMD, or CPS1 protein or functional CFTR, ATP7B, ATP7A, AGL, DMD, or CPS1 protein, but the increased levels of CFTR, ATP7B, ATP7A, AGL, DMD, or CPS1 protein or functional CFTR, ATP7B, ATP7A, AGL, DMD, or CPS1 protein do not exceed the levels of CFTR, ATP7B, ATP7A, AGL, DMD, or CPS1 protein or functional CFTR, ATP7B, ATP7A, AGL, DMD, or CPS1 protein expressed by the same cell type or tissue type in the reference control subject or population of reference control subjects. In some embodiments, expression of any of the vectors disclosed herein in the cell(s) or tissue(s) of the test subject results in an increase in levels of CFTR, ATP7B, ATP7A, AGL, DMD, or CPS1 protein or functional CFTR, ATP7B, ATP7A, AGL, DMD, or CPS1 protein, but the increased levels of CFTR, ATP7B, ATP7A, AGL, DMD, or CPS1 protein or functional CFTR, ATP7B, ATP7A, AGL, DMD, or CPS1 protein exceed the levels of CFTR, ATP7B, ATP7A, AGL, DMD, or CPS1 protein or functional CFTR, ATP7B, ATP7A, AGL, DMD, or CPS1 protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects. In some embodiments, any of the treatment and/or prophylactic methods disclosed herein are applied to a subject. In some embodiments, the subject is a mammal. In some embodiments, the subject is a human. In some embodiments, the human is a newborn, an infant, child, pre-adolescent, adolescent, or adult. In some embodiments, the human is less than 1 week of age, less than 2 weeks of age, less than one month of age, less than two months of age, less than 6 months of age, less than 1 year of age, less than 18 months of age, less than 2 years of age, less than 3 years of age, less than 4 years of age, less than 5 years of age, less than 10 years of age, less than 12 years of age, less than 16 years of age or less than 18 years of age.

### Cystic Fibrosis

In some embodiments, any of the treatment and/or prophylactic methods disclosed herein is for use in treatment of a patient having one or more mutations in the patient's Cystic Fibrosis Transmembrane Conductance Regulator (CFTR) gene.

Cystic Fibrosis (CF) is caused by mutations in the cystic fibrosis transmembrane conductance regulator (CFTR) gene which encodes a multi-membrane spanning epithelial chloride channel (Riordan et al., ANNU REV BIOCHEM 77, 701-26 (2008)). Approximately ninety percent of patients have a deletion of phenylalanine (Phe) 508 (ΔF508) on at least one allele. This mutation results in disruption of the energetics of the protein fold leading to degradation of CFTR in the endoplasmic reticulum (ER). The ΔF508 mutation is thus associated with defective folding and trafficking, as well as enhanced degradation of the mutant CFTR protein (Qu et al., J BIOL CHEM 272, 15739-44 (1997)). The loss of a functional CFTR channel at the plasma membrane disrupts ionic homeostasis (Cl−, Na+, HCO3−) and airway surface hydration leading to reduced lung function (Riordan et al.). Reduced periciliary liquid volume and increased mucus viscosity impede mucociliary clearance resulting in chronic infection and inflammation, phenotypic hallmarks of CF disease (Boucher, J INTERN MED 261, 5-16 (2007)). In addition to respiratory dysfunction, ΔF508 CFTR also impacts the normal function of additional organs (pancreas, intestine, gall bladder), suggesting that the loss-of-function impacts multiple downstream pathways that will require correction.

In addition to cystic fibrosis, mutations in the CFTR gene and/or the activity of the CFTR channel has also been implicated in other conditions, including for example, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), dry eye disease, Sjogren's syndrome and chronic sinusitis, cholestatic liver disease (e.g. Primary biliary cirrhosis (PBC) and primary sclerosing cholangitis (PSC)) (Sloane et al. (2012), PLoS ONE 7(6): e39809.doi:10.1371/journal. pone.0039809; Bombieri et al. (2011), J CYST FIBROS. 10 Suppl 2:S86-102; Albert et al. (2008), CLINICAL RESPIRATORY MEDICINE, Third Ed., Mosby Inc.; Levin et al. (2005), INVEST OPHTHALMOL VIS SCI., 46(4):1428-34; Froussard (2007), PANCREAS 35(1): 94-5), Son et al. (2017) J Med Chem 60(6):2401-10.

In some embodiments, CFTR activity is enhanced after administration of a nucleic acid described herein when there is an increase in the CFTR activity as compared to that in the absence of the administration of the nucleic acid. CFTR activity encompasses, for example, chloride channel activity of the CFTR, and/or other ion transport activity (for example, HCO3− transport). CFTR activity can be increased, for example, by at least 20%, at least 30%, at least 40%, at least 50%, at least 60%, at least 70%, at least 80%, at least 90%, at least 95%, or 100% upon administration of the nucleic acid.

Contemplated patients may carry a CFTR mutation(s) selected from ΔF508, S549N, G542X, G551D, R117H, N1303K, W1282X, R553X, 621+1G>T, 1717-1G>A, 3849+10kbC>T, 2789+5G>A, 3120+1G>A, 1507del, R1162X, 1898+1G>A, 3659delC, G85E, D1152H, R560T, R347P, 2184insA, A455E, R334W, Q493X, E56K, P67L, R74W, D110E, D110H, R117C, G178R, E193K, L206W, R347H, R352Q, A455E, S549R, G551S, D579G, S945L, S997F, F1052V, K1060T, A1067T, G1069R, R1070Q, R1070W, F1074L, G1244E, S1251N, S1255P, D1270N, G1349D, and 2184delA. Contemplated patients may carry a CFTR mutation(s) from one or more classes, such as without limitation, Class I CFTR mutations, Class II CFTR mutations, Class III CFTR mutations, Class IV CFTR mutations, Class V CFTR mutations, and Class VI mutations. TABLE 19 provides a description of each class of mutation.

TABLE 19

| | Effect on | Example of |
|---|---|---|
| Class | CFTR protein | mutation |
| I | Shortened protein | W1282X Instead of inserting the |
| | | amino acid tryptophan (W), the |
| | | protein sequence is prematurely |
| | | stopped (indicated by an X). |
| II | Protein fails to reach | ΔF508 A phenylalanine amino |
| | cell membrane | acid (F) is deleted |
| III | Channel cannot be | G551D A “missense” mutation: |
| | regulated properly | instead of a glycine amino |
| | | acid (G), aspartate (D) is added |
| IV | Reduced chloride | R117H Missense |
| | conductance | |
| V | Reduced due to incorrect | 3120 + 1G > A Splice-site |
| | splicing of gene | mutation in gene intron 16 |
| VI | Reduced due to protein | N287Y a A −> T at 991 |
| | instability | |

Contemplated subject (e.g., human subject) CFTR genotypes include, without limitation, homozygote mutations (e.g., ΔF508/ΔF508 and R117H/R117H) and compound heterozygote mutations (e.g., ΔF508/G551D; ΔF508/A455E; ΔF508/G542X; Δ508F/W1204X; R553X/W1316X; W1282X/N1303K, 591Δ18/E831X, F508del/R117H/N1303K/3849+10kbC>T; Δ303K/384; and DF508/G178R). TABLE 20 provides further description of selected genotypes.

TABLE 20

| Genotype | Description | Possible Symptoms |
|---|---|---|
| Δ508F/Δ508F | homozygote | Severe lung disease, |
| | | pancreatic insufficient |
| R117H/R117H | homozygote | Congenital bilateral absence |
| | | of the vas deferens, |
| | | No lung or pancreas |
| | | disease, |
| WT/Δ508F | heterozygote | Unaffected |
| WT/3120 + | heterozygote | Unaffected |
| 1 G > A | | |
| Δ508F/W1204X | compound | No lung disease, |
| | heterozygote | pancreatic insufficient |
| R553X and W1316X | compound | Mild lung disease, |
| | heterozygote | pancreatic insufficient |
| 591Δ18/E831X | compound | No lung or pancreas |
| | heterozygote | disease, nasal polyps |

In certain embodiments, the mutation is a Class I mutation, e.g., a G542X; a Class II/I mutation, e.g., a ΔF508/G542X compound heterozygous mutation. In other embodiments, the mutation is a Class III mutation, e.g., a G551D; a Class II/Class III mutation, e.g., a ΔF508/G551D compound heterozygous mutation. In still other embodiments, the mutation is a Class V mutation, e.g., a A455E; Class II/Class V mutation, e.g., a ΔF508/A455E compound heterozygous mutation.

Of the more than 1000 known mutations of the CFTR gene, ΔF508 is the most prevalent mutation of CFTR which results in misfolding of the protein and impaired trafficking from the endoplasmic reticulum to the apical membrane (Dormer et al. (2001). J Cell Sci 114, 4073-4081; http://www.genet.sickkids.on.ca/app). In certain embodiments, CFTR activity is enhanced (e.g., increased) following delivery of the nucleic acid to the subject. An enhancement of CFTR activity can be measured, for example, using literature described methods, including for example, Ussing chamber assays, patch clamp assays, and hBE Ieq assay (Devor et al. (2000), AM J PHYSIOL CELL PHYSIOL 279(2): C461-79; Dousmanis et al. (2002), J GEN PHYSIOL 119(6): 545-59; Bruscia et al. (2005), PNAS 103(8): 2965-2971).

As discussed above, the disclosure also encompasses a method of treating cystic fibrosis. Methods of treating other conditions associated with CFTR activity, including conditions associated with deficient CFTR activity, comprising administering an effective amount of a disclosed nucleic acid, are also provided herein.

For example, provided herein is a method of treating a condition associated with deficient or decreased CFTR activity comprising administering an effective amount of a disclosed nucleic acid. Non-limiting examples of conditions associated with deficient CFTR activity are cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, cholestatic liver disease (e.g. Primary biliary cirrhosis (PBC) and primary sclerosing cholangitis (PSC)), dry eye disease, protein C deficiency, Aβ-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

In some embodiments, disclosed methods of treatment further comprise administering an additional therapeutic agent. For example, in an embodiment, provided herein is a method of administering a disclosed nucleic acid and at least one additional therapeutic agent. In certain aspects, a disclosed method of treatment comprises administering a disclosed nucleic acid, and at least two additional therapeutic agents. Additional therapeutic agents include, for example, mucolytic agents, bronchodilators, antibiotics, anti-infective agents, anti-inflammatory agents, ion channel modulating agents, therapeutic agents used in gene therapy, CFTR correctors, and CFTR potentiators, or other agents that modulates CFTR activity. In some embodiments, at least one additional therapeutic agent is selected from the group consisting of a CFTR corrector and a CFTR potentiator. Non-limiting examples of CFTR correctors and potentiators include VX-770 (Ivacaftor), deuterated Ivacaftor, GLPG2851, GLPG2737, GLPG2451, VX-809 (3-(6-(1-(2,2-difluorobenzo[d][1,3]dioxol-5-yl)cyclopropanecarboxamido)-3-methylpyridin-2-yl)benzoic acid, deuterated lumacaftor, VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), VX-983, VX-152, VX-440, VX-445, VX-659, and Ataluren (PTC124) (3-[5-(2-fluorophenyl)-1,2,4-oxadiazol-3-yl]benzoic acid), FDL169, GLPG1837/ABBV-974 (for example, a CFTR potentiator), GLPG2665, GLPG2222 (for example, a CFTR corrector); and compounds described in, e.g., WO2014/144860 and 2014/176553, hereby incorporated by reference. Non-limiting examples of modulators include QBW-251, QR-010, NB-124, riociquat, SPX-101, and compounds described in, e.g., WO2014/045283; WO2014/081821, WO2014/081820, WO2014/152213; WO2014/160440, WO2014/160478, US2014027933; WO2014/0228376, WO2013/038390, WO2011/113894, WO2013/038386; and WO2014/180562, of which the disclosed modulators in those publications are contemplated as an additional therapeutic agent and incorporated by reference. Non-limiting examples of anti-inflammatory agents include N6022 (3-(5-(4-(1H-imidazol-1-yl) phenyl)-1-(4-carbamoyl-2-methylphenyl)-¹H-pyrrol-2-yl) propanoic acid), CTX-4430, N1861, N1785, and N91115.

The phrase “combination therapy,” as used herein, refers to an embodiment where a patient is co-administered a disclosed nucleic acid, and one or more of a CFTR potentiator agent (e.g., ivacaftor GLPG1837, GLPG2545, or GLPG3067) and a CFTR corrector agent(s) (e.g, VX-661, VX-152, VX-440, VX-445, VX659, GLPG2222, GLPG2851, GLPG2737 OR GLPG3221 and/or lumacaftor). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Sequential or substantially simultaneous administration of each therapeutic agent can be effected by any appropriate route including, but not limited to, oral routes, inhalational routes, intravenous routes, intramuscular routes, and direct absorption through mucous membrane tissues. The therapeutic agents can be administered by the same route or by different routes. For example, a first therapeutic agent of the combination selected may be administered by intravenous injection or inhalation or nebulizer while the other therapeutic agents of the combination may be administered orally. Alternatively, for example, all therapeutic agents may be administered orally or all therapeutic agents may be administered by intravenous injection, inhalation or nebulization.

### Wilson Disease (WD)

Wilson disease (WD) is an autosomal recessive genetic disorder that causes accumulation of copper primarily in the liver and subsequently in the neurological system and other tissues. WD is a rare disorder that affects approximately 1 in 30,000 individuals, caused by mutations in the copper transporting ATPase 2 (ATP7B) gene on chromosome 13. There are more than 600 unique ATP7B mutations. ATP7B is expressed mainly in hepatocytes and functions in the transmembrane transport of copper. Absent or reduced function of ATP7B protein results in decreased hepatocellular excretion of copper into bile, causing liver disease. Over time without proper treatment, high copper levels can cause life-threatening organ damage.

Patients with hepatic WD usually present in late childhood or adolescence, and exhibit features of acute hepatitis, fulminant hepatic failure, or progressive chronic liver disease. Neurologic manifestations of WD typically present later than the liver disease, most often in the second or third decade and include extrapyramidal, cerebellar, and cerebral-related symptoms.

The aim of medical treatment of WD is to remove the toxic deposit of copper from the body and to prevent its reaccumulation. Current treatment approaches for WD are daily oral therapy with chelating agents (D-penicillamine, trientine, and zinc salts). Medical therapy is effective in most, but not all WD patients. Liver transplantation is a therapeutic option in WD patients presenting with fulminant liver failure or progressive liver failure. However, transplant recipients are required to maintain a constant immune suppression regimen to prevent rejection. Further, compliance is a major issue for WD patients, and a single-dose cure would represent a substantial advancement.

In some embodiments, ATP7B activity is enhanced after administration of a nucleic acid described herein when there is an increase in the ATP7B activity as compared to that in the absence of the administration of the nucleic acid. ATP7B activity encompasses, for example, a decrease in free serum copper, and decrease in total serum copper, a decrease in 24-hour urinary copper, liver copper accumulation, and increase in serum cerulopasmin activity, or a decrease in liver pathology. ATP7B activity can be increased, for example, by at least 20%, at least 30%, at least 40%, at least 50%, at least 60%, at least 70%, at least 80%, at least 90%, at least 95%, or 100% upon administration of the nucleic acid.

Contemplated subjects include but are not limited to those carrying a G85V, L492S, G591D, A604P, R616W, G710S, P760L, D765N, M769V, L776V, R778Q, R778L, W779X, G943S, R969Q, T997M, V995A, P992L, E1064A, H1069Q, R1115H, and N1270S mutation.

As discussed above, the disclosure also encompasses a method of treating Wilson disease (WD) comprising administering to a subject a nucleic acid (e.g., a transgene) comprising an ATP7B coding sequence or functional fragment or variant thereof. Methods of treating other conditions associated with ATP7B activity, including conditions associated with deficient ATP7B activity, comprising administering an effective amount of a disclosed nucleic acid, are also provided herein.

In some embodiments, disclosed methods of treatment further comprise administering an additional therapeutic agent. For example, in an embodiment, provided herein is a method of administering a disclosed nucleic acid and at least one additional therapeutic agent. In certain aspects, a disclosed method of treatment comprises administering a disclosed nucleic acid, and at least two additional therapeutic agents. Additional therapeutic agents include, for example, chelating agents, such as penicillamine and trientine, and drugs to manage copper levels, such as zinc acetate.

### Menkes Disease and Occipital Horn Syndrome (OHS)

Menkes disease is an infantile onset X-linked recessive neurodegenerative disorder caused by deficiency or dysfunction of the copper-transporting ATPase ATP7A. As an X-linked disease, Menkes disease typically occurs in males who appear normal at birth, but present with loss of previously obtained developmental milestones and the onset of hypotonia, seizures and failure to thrive at 2 to 3 months of age. Characteristic physical changes of the hair and facies, in conjunction with typical neurologic findings, often suggest the diagnosis. The scalp hair of infants with classic Menkes disease is short, sparse, coarse, and twisted. Light microscopy of patient hair illustrates pathognomonic pili torti (for example, 180° twisting of the hair shaft) and the hair tends to be lightly pigmented and may demonstrate unusual colors, such as white, silver, or gray. The face of the individual with Menkes disease has pronounced jowls, with sagging cheeks and ears that often appear large. The palate tends to be high-arched, and tooth eruption is delayed. The skin often appears loose and redundant, particularly at the nape of the neck and on the trunk. Neurologically, profound truncal hypotonia with poor head control is almost invariably present. Developmental skills are confined to occasional smiling and babbling in most patients. Growth failure commences shortly after the onset of neurodegeneration and is asymmetric, with linear growth relatively preserved in comparison to weight and head circumference.

The biochemical phenotype in Menkes disease involves (1) low levels of copper in plasma, liver, and brain because of impaired intestinal absorption of copper, (2) reduced activities of numerous copper-dependent enzymes, and (3) paradoxical accumulation of copper in certain tissues (such as the duodenum, kidney, spleen, pancreas, skeletal muscle, and/or placenta). The copper-retention phenotype is also evident in cultured fibroblasts and lymphoblasts, in which reduced egress of radiolabeled copper is demonstrable in pulse-chase experiments.

Mouse models of Menkes disease are available and include the mottled mouse (Mercer, AM. J. CLIN. NUTR. 76:1022S-1028S, 1998; for example, brindled (mo-br), tortoise, dappled, viable-brindled, and/or blotchy (mo-blo) mice) and the macular mouse (e.g. Kodama et al., J. HISTOCHEM. CYTOCHEM. 41:1529-1535, 1993).

Occipital Hom Syndrome (OHS) is a milder allelic variant of Menkes disease. Serum copper levels are typically slightly below normal levels in OHS patients. OHS is characterized by wedge-shaped calcifications that form at the sites of attachment of the trapezius muscle and the sternocleidomastoid muscle to the occiput (“occipital homs”), which may be clinically palpable and/or visible on radiography. The phenotype of OHS includes slight generalized muscle weakness and dysautonomia (syncope, orthostatic hypotension, and chronic diarrhea). Subjects with OHS also typically have lax skin and joints, bladder diverticula, inguinal hernia, and vascular tortuosity. Intellect is usually normal or slightly reduced. Patients with hepatic WD usually present in late childhood or adolescence, and exhibit features of acute hepatitis, fulminant hepatic failure, or progressive chronic liver disease. Neurologic manifestations of WD typically present later than the liver disease, most often in the second or third decade and include extrapyramidal, cerebellar, and cerebral-related symptoms.

Contemplated subjects include but are not limited to those carrying a A629P, S637L, R844H, G860V, L873R, G876R, Q924R, C1000R, A1007V, G1015D, G1019D, D1044G, K1282E, G1300E, G1302V, N1304S, N1304K, D1305A, G1315R, A1325V, A1362D, A1362V, G1369R, S1397F mutation.

In some embodiments, ATP7A activity is enhanced after administration of a nucleic acid described herein when there is an increase in the ATP7A activity as compared to that in the absence of the administration of the nucleic acid. An increase in ATP7A activity can result in, for example, an increased level of copper in plasma, liver, or brain, an increase in activity of a copper-dependent enzymes, a reduction in accumulation of copper in the duodenum, kidney, spleen, pancreas, skeletal muscle, and/or placenta. An increase in ATP7A activity can also be measured in cultured fibroblasts and lymphoblasts by an increased egress of radiolabeled copper in pulse-chase experiments. ATP7A activity can be increased, for example, by at least 20%, at least 30%, at least 40%, at least 50%, at least 60%, at least 70%, at least 80%, at least 90%, at least 95%, or 100% upon administration of the nucleic acid.

As discussed above, the disclosure also encompasses a method of treating Menkes disease and OHS comprising administering to a subject a nucleic acid (e.g., a transgene) comprising an ATP7A coding sequence or functional fragment or variant thereof. Methods of treating other conditions associated with ATP7A activity, including conditions associated with deficient ATP7A activity, comprising administering an effective amount of a disclosed nucleic acid, are also provided herein.

In some embodiments, disclosed methods of treatment further comprise administering an additional therapeutic agent. For example, in an embodiment, provided herein is a method of administering a disclosed nucleic acid and at least one additional therapeutic agent. In certain aspects, a disclosed method of treatment comprises administering a disclosed nucleic acid, and at least two additional therapeutic agents. Additional therapeutic agents include, for example, administration of copper therapy by a parenteral (e.g., subcutaneous) route (see, e.g., Kaler et al., N. ENG. J. MED. 358:605-614, 2008; Kaler J. TRACE ELEM. MED. BIOL. 28:427-430, 2014). This additional therapy may be necessary due to defects in copper transport in the gut resulting from ATP7A defects, and leading to low serum copper levels. Thus, in some embodiments, a subject treated with the compositions disclosed herein (for example a subject with reduced serum copper levels, such as a subject having Menkes disease or OHS) is also treated with copper therapy.

The copper used for treatment may be in any form that can be conveniently administered and having an acceptable level of side effects (such as proximal renal tubular damage). In some examples, copper is in the form of copper histidine, copper histidinate, copper gluconate, copper chloride, and/or copper sulfate. In some examples, the copper is cGMP grade copper, such as cGMP grade copper histidinate. Generally a suitable dose is about 250 μg to about 500 μg of copper (such as copper histidinate, copper chloride, or copper sulfate) per day or every other day. However, other higher or lower dosages (or split doses) also could be used, such as from about 50 μg to about 1000 μg (such as about 50 μg to 200 μg, about 100 μg to 500 μg, about 250 μg to 750 μg, or about 500 μg to 1000 μg) per day or every other day, for example, depending on the subject age (e.g., infant, child, or adult) and body weight, route of administration, or other factors considered by a clinician. In some examples, subjects under 12 months of age are administered the copper in two daily doses, while subjects 12 months of age or older are administered the copper in a single daily dose. Copper therapy is administered by one or more parenteral routes, including, but not limited to subcutaneous, intramuscular, or intravenous administration. In one specific example, copper therapy (such as copper histidinate) is administered by subcutaneous injection.

The copper can be administered to the subject prior to, simultaneously, substantially simultaneously, sequentially, or any combination thereof, with the ATP7A nucleic acid, vector, recombinant virus, or composition described herein. In some examples, at least one dose of copper is administered within 24 hours of administration of the ATP7A nucleic acid, vector, recombinant virus, or composition described herein. Additional doses of copper can be administered at later times, as selected by a clinician. In some examples, copper therapy is administered daily for at least 3 months, at least 6 months, at least 1 year, at least 2 years, at least 3 years or more (such as about 3 months to 5 years, 6 months to 3 years, 1 to 2 years, 2 to 6 years, or 1 to 5 years). Copper therapy (such as daily administration of copper) may begin immediately upon diagnosis of a subject with an ATP7A-related copper transport disorder and in some examples may occur prior to administration of the ATP7A nucleic acid, vector, recombinant virus, or composition described herein, and also continue daily following ATP7A nucleic acid administration. One of ordinary skill in the art can also select additional treatments for subjects with an ATP7A-related copper transport disorder, such as L-threo-dihydroxyphenylserine (L-DOPS, also known as droxidopa).

In some embodiments, the effectiveness of treatment of a subject with an ATP7A or an ATP7B nucleic acid as disclosed herein is evaluated by determining one or more biochemical markers of copper metabolism in a sample from a subject (such as serum or CSF copper level, serum ceruloplasmin level, plasma or CSF catecholamine levels, or cellular copper egress). Methods of detecting these biochemical markers of copper metabolism are well known in the art.

In some examples, a value for a biochemical marker of copper metabolism (such as copper level, ceruloplasmin level, catecholamine level, or cellular copper egress) in a sample from a subject is compared to a value for the same marker from a control (such as a reference value, a control population, or a control individual). In some examples, a control is a subject with untreated ATP7A- or ATP7B-related copper transport disorder (or a reference value or a control population with untreated ATP7A- or ATP7B-related copper transport disorder). In other examples, a control is a healthy subject (such as a subject that does not have a copper transport disorder) or a reference value or healthy control population. In some examples, the control may be samples or values from the subject with the ATP7A- or ATP7B-related copper transport disorder, for example, prior to commencing treatment.

One biochemical marker of copper metabolism is the level of copper in a sample from a subject (such as serum, plasma, or CSF). In some examples, reduced copper level as compared to a normal control individual or normal control population is a marker of Menkes disease or OHS. Methods of determining copper levels in a sample (such as serum, plasma, or CSF from a subject) are well known to one of skill in the art. In some examples, methods for determining copper levels in a sample include flame atomic absorption spectrometry, anodic stripping voltammetry, graphite furnace atomic absorption, electrothermal atomic absorption spectrophotometry, inductively coupled plasma-atomic emission spectroscopy, and inductively coupled plasma-mass spectrometry. See, e.g. Evenson and Warren, CLIN. CHEM. 21:619-625, 1975; Weinstock and Uhlemann, CLIN. CHEM. 27:1438-1440, 1981; WO 93/017321.

Ceruloplasmin is the major copper-carrying protein in the blood. This protein has ferroxidase and amine oxidase activity and catalyzes the enzymatic oxidation of p-phenylenediamine (PPD) and Fe(II). Levels of ceruloplasmin in a sample from a subject (such as serum, plasma, or CSF) are a biochemical marker of copper metabolism. In some examples, reduced ceruloplasmin level as compared to normal control sample or population or a reference value is a marker of Menkes disease or OHS.

Methods of determining ceruloplasmin levels in a sample (such as serum, plasma, or CSF) are well known to one of skill in the art. In one example, ceruloplasmin levels in a sample are determined by measuring ceruloplasmin oxidase activity (such as PPD-oxidase activity or ferroxidase activity). See, e.g., Sunderman and Nomoto, CLIN. CHEM. 16:903-910, 1970). The rate of formation of oxidation product is proportional to the concentration of serum ceruloplasmin (with a correction for non-enzymatic oxidation of substrate). In another example, ceruloplasmin levels in a sample are determined by immunoassay, such as ELISA, dissociation-enhanced time-resolved fluoroimmunoassay, or turbidimetric immunoassay (see, e.g., U.S. Pat. Nos. 6,806,044; 6,010,903; 5,491,066). In a further example, ceruloplasmin levels are determined by purifying ceruloplasmin and analyzing the copper content using inductively coupled plasma mass spectroscopy to provide a copper ion specific signal; and the sample is evaluated for ceruloplasmin based on the copper ion specific signal (see, e.g., U.S. Pat. Publication No. 2007/0161120).

### Cori Disease

Glycogen Storage Disease type III, or Cori Disease (also Forbes Disease), is caused by a deficiency in glycogen debrancher enzymes, leading to abnormally structured glycogen accumulates in the liver, skeletal and cardiac muscles. Initial presentation occurs at a young age and commonly involves hepatomegaly, fasting hypoglycemia and hyperlipidemia. Despite dietary modifications, progressive liver disease often results in progressive liver fibrosis leading to cirrhosis, with the risk of developing hepatocellular carcinoma and/or liver failure. Long-term complications may also involve dilative cardiomyopathy or life-threatening arrhythmias and death.

Over 120 pathogenic mutations or likely pathogenic mutations in AGL have been identified for Cori Disease. There are estimated to be ˜10,000 patients worldwide.

Cori Disease patients may suffer from skeletal myopathy, cardiomyopathy, cirrhosis of the liver, hepatomegaly, hypoglycemia, short stature, dyslipidemia, slight mental retardation, facial abnormalities, and/or increased risk of osteoporosis (Ozen et al. (2007) WORLD J GASTROENTEROL, 13(18): 2545-46). Forms of Cori Disease with muscle involvement may present muscle weakness, fatigue and muscle atrophy. Progressive muscle weakness and distal muscle wasting frequently become disabling as the patients enter the third or fourth decade of life, although this condition has been reported to begin in childhood in many Japanese patients.

In certain embodiments, “treatment” of Cori Disease encompasses a complete reversal or cure of the disease, or any range of improvement in conditions and/or adverse effects attributable to Cori Disease. Merely to illustrate, treatment of Cori Disease includes an improvement in any of the following effects associated with Cori Disease or combination thereof: skeletal myopathy, cardiomyopathy, cirrhosis of the liver, hepatomegaly, hypoglycemia, short stature, dyslipidemia, failure to thrive, mental retardation, facial abnormalities, osteoporosis, muscle weakness, fatigue and muscle atrophy. Treatment may also include one or more of reduction of abnormal levels of cytoplasmic glycogen, decrease in elevated levels of one or more of alanine transaminase, aspartate transaminase, alkaline phosphatase, or creatine phosphokinase, such as decrease in such levels in serum. Improvements in any of these conditions can be readily assessed according to standard methods and techniques known in the art. Other symptoms not listed above may also be monitored in order to determine the effectiveness of treating Cori Disease. The population of subjects treated by the method of the disease includes subjects suffering from the undesirable condition or disease, as well as subjects at risk for development of the condition or disease.

In some embodiments, AGL activity is enhanced after administration of a nucleic acid described herein when there is an increase in the AGL activity as compared to that in the absence of the administration of the nucleic acid. An increase in AGL activity can result in, for example, (1) an improvement in one or more of skeletal myopathy, cardiomyopathy, cirrhosis of the liver, hepatomegaly, hypoglycemia, short stature, dyslipidemia, failure to thrive, mental retardation, facial abnormalities, osteoporosis, muscle weakness, fatigue and muscle atrophy and/or (2) a reduction in abnormal levels of cytoplasmic glycogen and/or a decrease in elevated levels of one or more of alanine transaminase, aspartate transaminase, alkaline phosphatase, or creatine phosphokinase, such as decrease in such levels in serum. AGL activity can be increased, for example, by at least 20%, at least 30%, at least 40%, at least 50%, at least 60%, at least 70%, at least 80%, at least 90%, at least 95%, or 100% upon administration of the nucleic acid.

Contemplated subjects include but are not limited to those carrying a V109L, W248, W461, R494H, R524H, R578S, L620Vfs, K819fs, R910, Y969Lfs, E1072Dfs, Q1159fs, Q1162, Q1209, R1272fs, K1302fs, W1327, and Y1510 mutations.

In addition, a nucleic acid (e.g., a transgene) comprising an AGL coding sequence or a functional fragment or variant thereof as described herein can be administered alone or in combination with one or more additional compounds or therapies for treating Cori Disease. For example, one or more nucleic acids can be co-administered in conjunction with one or more therapeutic compounds. When co-administration is indicated, the combination therapy may encompass simultaneous or alternating administration. In addition, the combination may encompass acute or chronic administration. Optionally, the nucleic acids of the present disclosure and additional compounds act in an additive or synergistic manner for treating Forbes-Cori Disease. Additional compounds to be used in combination therapies include, but are not limited to, small molecules, polypeptides, antibodies, antisense oligonucleotides, and siRNA molecules.

In another example of combination therapy, a nucleic acid of the disclosure can be used as part of a therapeutic regimen combined with one or more additional treatment modalities. By way of example, such other treatment modalities include, but are not limited to, dietary therapy, occupational therapy, physical therapy, ventilator supportive therapy, massage, acupuncture, acupressure, mobility aids, assistance animals, and the like. Current treatments of Cori disease include diets high in carbohydrates and cornstarch alone or with gastric tube feedings. Patients having myopathy also are traditionally fed high-protein diets. The nucleic acids of the present disclosure may be administered in conjunction with these dietary therapies. In other embodiments, the methods of the disclosure reduce the need for the patient to be on the dietary regimen.

In certain embodiments, one or more nucleic acids of the present disclosure can be administered prior to or following a liver transplant.

Note that although the nucleic acids described herein can be used in combination with other therapies, in certain embodiments, a nucleic acid is provided as the sole form of therapy.

### Duchenne Muscular Dystrophy (DMD)

Duchenne muscular dystrophy (DMD) is a severe type of muscular dystrophy that primarily affects boys. Muscle weakness usually begins around the age of four, and worsens quickly. Muscle loss typically occurs first in the thighs and pelvis followed by the arms. This can result in trouble standing up. Most are unable to walk by the age of 12. Affected muscles may look larger due to increased fat content. Scoliosis is also common. Some may have intellectual disability. Females with a single copy of the defective gene may show mild symptoms.

The disorder is X-linked recessive. About two thirds of cases are inherited from a person's mother, while one third of cases are due to a new mutation. It is caused by a mutation in the gene for the protein dystrophin. Dystrophin is important to maintain the muscle fiber's cell membrane. Genetic testing can often make the diagnosis at birth. Those affected also have a high level of creatine kinase in their blood.

Although there is no known cure, physical therapy, braces, and corrective surgery may help with some symptoms. Assisted ventilation may be required in those with weakness of breathing muscles. Medications used include steroids to slow muscle degeneration, anticonvulsants to control seizures and some muscle activity, and immunosuppressants to delay damage to dying muscle cells.

DMD affects about one in 3,500 to 6,000 males at birth. It is the most common type of muscular dystrophy. The average life expectancy is 26; however, with excellent care, some may live into their 30s or 40s. The disease is much more rare in girls, occurring approximately once in 50,000,000 live female births.

Contemplated subjects include but are not limited to those carrying a deletion, e.g., a deletion of one or more exons, a duplication (e.g., a duplication of one or more whole exons), an a point mutation.

In some embodiments, DMD activity is enhanced after administration of a nucleic acid described herein when there is an increase in the DMD activity as compared to that in the absence of the administration of the nucleic acid. An increase in DMD activity can result in, for example, improved muscle strength and/or tone, increase in muscle size, improvement in cardiomyopathy, lowered serum creatine kinase, and/or improved respiration. DMD activity can be increased, for example, by at least 20%, at least 30%, at least 40%, at least 50%, at least 60%, at least 70%, at least 80%, at least 90%, at least 95%, or 100% upon administration of the nucleic acid.

As discussed above, the disclosure also encompasses a method of treating DMD comprising administering to a subject a nucleic acid (e.g., a transgene) comprising an DMD coding sequence or functional fragment or variant thereof. Methods of treating other conditions associated with DMD activity, including conditions associated with deficient DMD activity, comprising administering an effective amount of a disclosed nucleic acid, are also provided herein.

In some embodiments, disclosed methods of treatment further comprise administering an additional therapeutic agent. For example, in an embodiment, provided herein is a method of administering a disclosed nucleic acid and at least one additional therapeutic agent. In certain aspects, a disclosed method of treatment comprises administering a disclosed nucleic acid, and at least two additional therapeutic agents. Additional therapeutic agents include, for example, eteplirsen (Exondys 51), deflazacort (Emfaza), prednisone, and golodirsen (Vyondys 53).

In some embodiments, the effectiveness of treatment of a subject with a DMD nucleic acid as disclosed herein is evaluated by determining one or more of muscle strength and/or tone, muscle size, cardiomyopathy, serum creatine kinase, and/or respiration using methods known in the art.

### Carbamoyl Phosphate Synthetase I Deficiency (CPS1D)

Carbamoyl phosphate synthetase I deficiency (CPS1D) is a rare, severe disorder of urea cycle metabolism. There are 2 main forms: (i) a lethal neonatal type characterized by severe hyperammonemia, manifesting in newborns with lethargy, vomiting, hypothermia, seizures, coma and death, and (ii) a typically milder and less severe, delayed-onset type.

CPS1D is a genetic disorder caused by mutations in the CPS1 gene and is inherited in an autosomal recessive fashion. The CPS1 gene encodes carbamoyl-phosphate synthase I (CPS1), an enzyme located in the mitochondrial matrix of hepatocytes and epithelial cells of intestinal mucosa, and controls the first step of the urea cycle where ammonia is converted into carbamoyl-phosphate. Mutations in this gene lead to an interruption in the urea cycle such that excess nitrogen is not converted to urea for excretion by the kidneys, leading to hyperammonemia. Patients with CPS1D can exhibit aminoaciduria (high urine amino acid levels), episodic ammonia intoxication, hyperammonemia (high blood ammonia levels), hypoarginemia (low blood arginine levels), and/or muscular hypotonia (low or weak muscle tone).

Contemplated subjects include but are not limited to those carrying a A43V, G58D, S65F, V71G, G79E, P87S, Y87S, Y89D, S123F, D165G, Y212N, D224V, R233C, H243P, G258E, G263E, K280N, G301E, A304V, G317E, H337R, N355D, D358H, P382L, Y389C, L390R, G401R, G431R, G432V, A438T, A438P, K450E, V457G, T471N, A498P, V531E, V531G, T544M, R587C, R587H, R587L, A589T, G593R, S597L, V622M, G628D, 1632R, R638P, A640S, C648Y, E651K, D654V, N674I, N674K, Q678P, N698S, N716K, R718K, R721Q, A724P, A726T, D767V, P774L, R780H, M7921, R803S, R803G, pR803C, F805L, F805S, Q810R, R814W, C816R, L843S, R850C, R850H, K875E, G911E, G911V, S913L, D914H, D914G, S918P, R932T, A949T, L958P, Y959C, Y962C, V978E, G982S, G982D, G982V, Y984H, 1986T, G987C, F992S, S998F, N1016S, P1017L T10221, E1034G, H1045R, 11054R, Q1059R, A1065E, R1089C, R1089L, Q1103R, V1141G, A1155E, A1155V, H1195P, S1203P, S1203L, D1205N, I1215V, R1228Q, N1241K, E1255D, R1262Q, R1262P, D1274H, C1327R, S1331P, G1333E, R1371L, A1378T, L1381S, T1391M, L1398V, P1411L, P1439L, T1443A, R1453W, R1453Q, P1462R, Y1491H, Q44X, Y89X, Y140X, R238X, Q375X, Q478X, E539X, Y590X, R721X, R787X, Q965X, Y1031X, W1106X, R1174X, R1262X, K42RfsX15, D52LfsX3, S137IfsX2, G177RfsX25, L234CfsX2, L244X, E283GfsX16, K287RfsX12, P289HfsX10, G301EfsX24, K399EfsX22, P464CfsX7, N472QfsX2, V474EfsX15 (Donor splice site error), G510AfsX5, A613FfsX25 (Acceptor splice site error), H659TfsX22, A613VfsX20 (Donor splice site error), A717AfsX28, A724PfsX27, L725WfsX19, L743X, K751RfsX42, C761X, R780VfsX13, G802VfsX19, E832VfsX5, E832KfsX9, A907PfsX25, L933X, I937PfsX5, Y959CfsX9, Y962SfsX11 (Donor splice site error), E966AfsX27, C1015WfsX4, N1062TfsX38, N1113WfsX10 (Acceptor splice site error), E1114DX, K1120VfsX25, H1162TfsX5, R1228FfsX24, E1290DfsX12, D1322AfsX5, I1324HfsX5, I1324PfsX3, Q1368SfsXl7, N1383MfsX44, Q1468SfsX8, L1426X (Donor splice site error), V1469IfsX2 mutation. Contemplated subjects also include, but are not limited to, those carrying a genomic change as follows, wherein nucleotide numbers correspond to the reference cDNA sequence NG_008285.1 from GenBank giving the +1 value to the A of the ATG translation initiation codon: c.236+6T>C (mRNA change not determined); c.306_311dupGAATGG; c.471+1G>A (c.382_271delExon4); c.529-3T>G (mRNA change not determined); c.622-7A>G (c.621_622insTGGCAG); (No DNA change identified) c.622_711delExon7; c.711+1G>A (c.622_711delExon7); c.711+686_1164+136del4260 (c.712-1086delExons 8-10); c.840G>C (c.832_840delGTCAGAAAG); c.1087-1G>T (mRNA change not determined); c.1164+1G>A (c.1087_1164delExon11); c.1263+5G>C (c.1165-1263delExon12); c.2895+1G>A (c.2830_2895delExon23); c.2995_2997delAGT; c.3036_3038delGGT; c.3159_3161delCAT; c.3558+1G>C (c.3481_3558delExon29); c.3559-2A>G (c.3559_3666delExon30); c.3756+1G>A (c.3667-3756delExon31); and c.4088_4099del12; c.4101+2T>C (c.4003_4101delExon34+c.4101_4102ins42).

In some embodiments, CPS1 activity is enhanced after administration of a nucleic acid described herein when there is an increase in the CPS1 activity as compared to that in the absence of the administration of the nucleic acid. An increase in CPS1 activity can result in, for example, (1) a decrease in urine amino acid levels, frequency of episodic ammonia intoxication, and/or blood ammonia levels, or (2) an increase in blood arginine levels and/or muscle tone.

As discussed above, the disclosure also encompasses a method of treating CPS1D comprising administering to a subject a nucleic acid (e.g., a transgene) comprising an CPS1 coding sequence or functional fragment or variant thereof. Methods of treating other conditions associated with CPS1 activity, including conditions associated with deficient CPS1 activity, comprising administering an effective amount of a disclosed nucleic acid, are also provided herein. CPS1 activity can be increased, for example, by at least 20%, at least 30%, at least 40%, at least 50%, at least 60%, at least 70%, at least 80%, at least 90%, at least 95%, or 100% upon administration of the nucleic acid.

In some embodiments, disclosed methods of treatment further comprise administering an additional therapeutic agent. For example, in an embodiment, provided herein is a method of administering a disclosed nucleic acid and at least one additional therapeutic agent. In certain aspects, a disclosed method of treatment comprises administering a disclosed nucleic acid, and at least two additional therapeutic agents. Additional therapeutic agents include, for example, glycerol phenylbutrate.

In some embodiments, the effectiveness of treatment of a subject with a CPS1 nucleic acid as disclosed herein is evaluated by determining one or more of urine amino acid levels, frequency of episodic ammonia intoxication, blood ammonia levels, blood arginine levels and/or muscle tone. Methods of detecting these biochemical markers and/or phenotypes of urea metabolism are well known in the art.

### IX. Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1. Therapeutic Development of Compact Promoters for CFTR-AAV Gene Therapy

This Example describes identification and characterization of a promoter that is small, strong, ubiquitous, and endogenous, for adeno-associated virus (AAV) CFTR packaging.

Bioinformatics analysis revealed the H1 bidirectional promoter appears to be ubiquitously expressed, which is logical given the biology and tissue expression data for both H1-driven genes (H1RNA and PARP-2). Endogenously, the H1 bidirectional promoter expresses an essential RNA gene (H1RNA) involved with tRNA processing and a ubiquitously expressed protein gene (PARP2). While a lack of transgene silencing using the H1 bidirectional promoter is not guaranteed, this result would be consistent with other endogenous mammalian promoters.

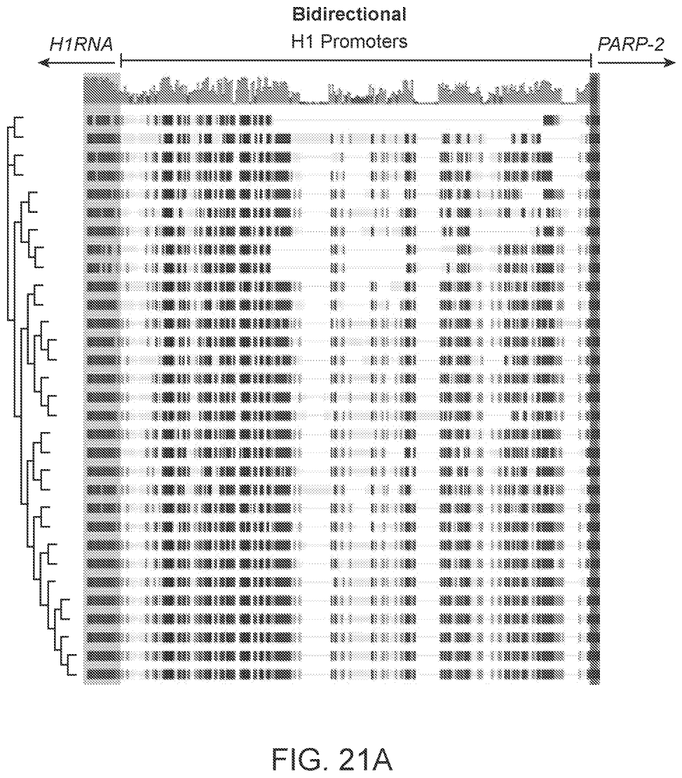

Evolutionary conservation throughout eutherian mammals further supports the presence of a functional genetic control element between the H1RNA and PARP2 genes, and enabled identification of numerous small and compact promoters through gene synteny (FIG. 21A). The orthologous H1 bidirectional promoters tested have all shown promoter activity in human cell lines, as well as cell lines of multiple different species.

To test the relative strength of the numerous promoter orthologs, a luciferase reporter construct that enables quantitation of RNA polymerase II (pol II) promoter activity was designed. In order to reduce any confounding noise and spurious reporter gene transcription, the plasmid constructs contained 5′ and 3′ beta-globin insulators that flank the expression cassette; the H1 promoter, firefly luciferase, and bGH poly(A) signal were found inside the insulators. It was observed that the pol II promoter activity varied significantly between orthologs, and consequently, the analysis was expanded to over 70 promoters, each tested in multiple human cell lines (FIG. 21B). The constructs were fully-synthesized, sequence verified, and amplified by endotoxin-free maxipreps for transfection studies.

In order to benchmark the pol II expression levels of these H1 promoters against known promoters, two commonly used promoters were included, the HSK thymidine kinase (TK) promoter and the phosphoglycerate kinase 1 (PGK1) promoter. The TK promoter is 753 basepairs (bp) and known to be a promoter that drives lower expression levels of regulated genes, while PGK1 is 515 bp and known to drive higher expression of regulated genes. The data in FIG. 21B shows the ranked order of promoter activity in HeLa cells with TK (orange, 8th bar from the left) and PGK1 (blue, 1st bar from the right) indicated. FIG. 21B demonstrates a wide range of expression of the H1 promoter orthologs.

Additionally, the promoter lengths were plotted overlaying the same data with red bars and corresponding to the right Y axis (a non-standard Y-axis range of 150 bp to 250 bp was used to depict the sizes for each promoter clearly). In addition to a range of activity, the promoter sizes were small (between about 150-240 bp) and demonstrated no correlation between size and promoter activity. Indeed, multiple promoters were found in the 150-180 bp size range with significant transcriptional activity. Nine of the promoters were 183 bp (the size of the F5tg83 promoter) or smaller.

A previous report comparing the promoter activity of the ITR alone (the promoter used in the original CFTR gene therapy trials), against two versions of a minimal TK promoter TK1 (110 bp) and TK2 (102 bp), found that the minimal TK promoters had over 10-fold higher promoter activity (Wang, D. et al., 1999. Efficient CFTR expression from AAV vectors packaged with promoters—the second generation. Gene Ther 6, 667-675.). Additionally, these minimal TK promoters exhibited approximately 25% of the expression of a strong 650 bp CMV immediate-early promoter. Given that these were truncated TK promoters, it would seem reasonable for promoter activity to be reduced compared to the full-length promoter in these experiments. Nevertheless, the full-length TK promoter marked the lower portion of the promoter activity range. Thus, the compact endogenous promoters are likely to be substantially stronger than those previously engineered for AAV-CFTR delivery.

Key challenges for CFTR gene therapy with AAV are addressed by these novel promoters: they are small and compact, ubiquitously expressed, provide a large range of expression, and are both endogenous and mammalian derived, making them suitable for therapeutic purposes.

### Example 2. CFTR Expression Driven Through Candidate Promoters

This Example describes a comparison of multiple expression constructs for their capacity to drive CFTR expression in A549 and HEK293 cells, two cell lines that do not endogenously express CFTR. mRNA and protein levels analyses are used to determine both the presence and expression levels resulting initially from plasmid constructs and then subsequently through packaged viruses.

### Promoter Selection

The most likely explanation for the failure to detect CFTR mRNA in clinical trials is due to the lack of promoter activity. While taking into account length, expression analysis, and other factors, seven candidate promoters are selected based on the reporter assay data previously generated in multiple cell lines, including HEK293. The candidate promoters represent a range of sizes, primarily focusing on those orthologs that are <200 bp. Similarly, the candidate promoters comprise a range of strengths, as determined previously by the luciferase promoter screens in multiple human cell lines (as described in Example 1 above). Additionally, two control promoters—EF1a and F5tg83—are included to provide positive controls for both gene expression and benchmarking levels.

### Plasmid Constructs and Cloning

The wild-type (WT) CFTR coding sequence are synthesized and cloned downstream of the selected promoters. For initial studies, the short synthetic poly(A) (˜50 nt) terminator sequence is used. These constructs are designed to contain the same expression cassette that would be subsequently be packaged for viral experiments. The constructs also contain flanking NotI restriction sites for cloning into a custom designed plasmid containing flanking AAV2 ITR sequences. A total of ten CFTR plasmid constructs, including positive and negative controls, are synthesized. Following sequencing verification and amplification by endotoxin-free maxipreps, the plasmids are used for transfection studies.

### Plasmid Transfection

Transfection experiments are conducted in HEK293 and A549 cells, which are cell lines that are readily transfected using lipid-based reagents. The A549 cell line is derived from a lung carcinoma; however, these cells do not endogenously express CFTR. Transfection conditions are tested to ensure that plasmid DNA content correlates with expression. Cells are then sub-cultured for 3-5 passages and seeded into multi-well plates. At 24 hours after seeding, cells are transfected with plasmids using Lipofectamine 3000.

### RT-qPCR

WT CFTR mRNA expression is determined by RT-qPCR analysis. HEK293 and A549 cell lines do not express CFTR and this absence of endogenous expression simplifies the initial assessment of transgene expression. At 48 hours post transfection, cells are lysed in QuickExtract RNA and treated with DNase I according to the manufacturer's protocol (Lucigen). Cells are treated again with DNase I (Turbo DNase I, Invitrogen) to remove any traces of contaminating DNA, and then samples are column purified (Zymo RNA Clean & Concentrator). RNA is quantitated and between 20 ng-2 μg per sample are used for cDNA synthesis with random primers; all reactions include a minus-reverse transcriptase (RT) control (−RT) (High-Capacity cDNA Reverse Transcription Kit, ThermoFisher). cDNA reactions and −RT controls are then used as templates for qPCR reactions containing CFTR primer-pair probes (ThermoFisher or IDT) and control human GAPDH predesigned primer-pair TaqMan assays (ThermoFisher) with a 2× TaqMan Multiplex Master Mix (ThermoFisher). RT-qPCR analysis is only valid from samples without amplification in the −RT reactions. Following determination of the precise protocol, which encompasses determination of conditions for singleplex and multiplex TaqMan assays, the blinded samples are sent to an external contract research organization and processed according to the numbers as detailed in TABLE 21.

TABLE 21
Summary of processing protocol for RT-qPCR analysis of CFTR plasmids
CFTR RT-qPCR Analysis

| | | Technical | Biological | Cell | Reac- |
|---|---|---|---|---|---|
| Sample | Constructs | Replicates | Replicates | lines | tions |
| −RT | 10 | 1 | 3 | 2 | 60 |
| +RT | 10 | 4 | 3 | 2 | 240 |

### Western Blot

To determine the protein levels of WT CFTR, western blot analysis in HEK293 cells following methods known in the art are utilized. Briefly, at 48 h post-transfection, cells are lysed in RIPA buffer containing protease inhibitors. 10-30 μg of lysate are run on SDS-PAGE gels (Invitrogen), transferred to PVDF membranes, and probed with antibodies against CFTR (ab596, CFF Antibody Distribution Program) and R-actin as control. Western blot band intensities are then quantified, noting the B band and C band corresponding to the core glycosylated and mature glycosylated forms of CFTR, respectively. In order to reduce the total number of samples, five constructs are chosen, including the two controls for protein analysis and one cell line are analyzed. A summary of the analysis is shown in TABLE 22.

TABLE 22
Summary of processing strategy for protein analysis of CFTR
CFTR Western Blot Analysis

| | | Technical | Biological | Cell | Reac- |
|---|---|---|---|---|---|
| Sample | Constructs | Replicates | Replicates | lines | tions |
| Protein | 5 | 3 | 3 | 1 | 45 |

Plasmid expression of CFTR as determined by RT-qPCR and western blotting is analyzed to provide an initial assessment of promoter activity and CFTR expression. As described above, over 70 small promoters may be utilized, and replace promoters that may exhibit little or no transcriptional activity. Given that the promoters have previously been tested for activity in HEK293 cells, the existing reporter assay data provide a good starting point for such analysis.

Detection of expression at this stage would provide a strong rationale for AAV packaging and further experimentation. CFTR expression data is used to select three promoter constructs (plus two controls) to be packaged into AAV vectors for subsequent experiments.

### AAV Packaging

For the in vitro experiments, the AAV-DJ will be utilized as the viral vector, which exhibits higher in vitro transduction efficiency than other wild-type serotypes (Grimm, D. et al. 2008. In vitro and in vivo gene therapy vector evolution via multispecies interbreeding and retargeting of adeno-associated viruses. J VIROL 82, 5887-5911). Constructs are cloned into ITR-containing plasmids, and small-scale packaging is used to produce virus for initial in vitro transduction analysis. Small scale preparations are produced.

The process is briefly summarized here: HEK293T cells are used in triple-transfected reactions, with an ITR-containing transfer plasmid, a rep and AAV-DJ serotype-specific cap plasmid, and a helper plasmid encoding the adenoviral sequences. At 72 hours following transfection, cells are collected and subjected to three freeze-thaw cycles to release viral particles. Following centrifugation to remove cell debris, the supernatant is stored at −80° C. Crude lysate will be titered by digital PCR (dPCR) using a known ITR primer and probe combination and quantified on a QuantStudio 3D Digital PCR System (Thermo Scientific).

### AAV Transduction

AAV transduction is first tested for culture conditions using a control vector. Serial dilutions of packaged AAV-DJ constructs are added to the cell culture media of HEK293 and A549 cells, with multiplicity of infections (MOI) ranging from 1×10³-1×10⁵. After 2 hours, the cells are washed, and media replaced. After 72 hours, transduction efficiency is compared across the range of MOIs using dPCR. Based on the optimized MOI conditions, the five AAV constructs are transduced, and CFTR mRNA and protein expression is quantified at 72 hours post-transduction.

### RT-qPCR

Following transduction, cells are processed for CFTR mRNA expression, as described above. A summary of the analysis is shown in TABLE 23.

TABLE 23
Summary of processing protocol for RT-qPCR analysis of CFTR mRNA
CFTR RT-qPCR Analysis

| | | Technical | Biological | Cell | Reac- |
|---|---|---|---|---|---|
| Sample | Constructs | Replicates | Replicates | lines | tions |
| −RT | 5 | 1 | 3 | 2 | 30 |
| +RT | 5 | 4 | 3 | 2 | 120 |

### Western Blot

Following transduction, cells are processed for CFTR protein expression, as described above. A summary of the analysis is shown in TABLE 24.

TABLE 24
Summary of processing strategy for protein analysis of CFTR
CFTR Western Blot Analysis

| | | Technical | Biological | Cell | Reac- |
|---|---|---|---|---|---|
| Sample | Constructs | Replicates | Replicates | lines | tions |
| Protein | 5 | 3 | 3 | 1 | 45 |

Viral packaging and delivery are essential for therapeutic rescue of deficient CFTR in patients. RT-qPCR and western blot analysis are used for confirmation of the presence of CFTR expression derived from CFTR-AAV constructs.

### Example 3. Analysis of Lead Constructs in AAV-Packaged ALI Cultures

This Example describes further analysis of two of the candidate CFTR constructs that are first tested in 2D cultures, as described in Example 2 above, under more physiologically relevant, therapeutic conditions.

Two constructs are assembled with ITR and packaged into AAV6, which is the AAV serotype of greatest potential for therapeutic delivery. These packaged constructs are delivered to CFTR-deficient cells (CFBE4lo−) cultured at an air-liquid interface to best mimic physiological conditions. Transduced cells are assessed for CFTR mRNA expression and functionality, such as transepithelial Cl⁻ transport. CFBE4lo− cells stably expressing a wild-type CFTR minigene (MG) are used as positive controls. AAV6-packaged constructs are then tested in patient-derived nasal epithelial cells to quantify restoration of CFTR expression and function.

### Air-Liquid Interface Cell Cultures.

CFBE4lo− airway epithelial cells and positive control cells are seeded onto polyester transwell inserts (0.4 μm pore size) that have been precoated with collagen/BSA/fibronectin and differentiated under air-liquid interface (ALI) conditions, as previously described (Sharma, N. et al. 2018. Capitalizing on the heterogeneous effects of CFTR nonsense and frameshift variants to inform therapeutic strategy for cystic fibrosis. PLoS GENET 14). Cells are cultured for approximately 2-4 weeks and evaluated for phenotypic hallmarks of a confluent polarized monolayer, including an increase in transepithelial electrical resistance (approximately 200-600 Ω/cm²), as determined by an epithelial voltohmmeter (EVOM, World Precision Instruments Inc).

### AAV Transduction of CFBE4lo− Cells

Serial dilutions of packaged AAV6 constructs are added to the apical surface, with MOI ranging from 1×10³-1×10⁵. After 2 hours, the apical surface of cells is washed, and media is replaced. After 2 weeks, transduction efficiency is compared across the range of MOIs. These conditions are also used for subsequent experiments.

### RT-qPCR

Following transduction, cells are processed for mRNA expression as described for 2D cultures above. mRNA expression is measured at 3 time points after transduction (2, 3, 4 weeks). A summary of the analysis is shown in TABLE 25.

TABLE 25
Summary of processing protocol for RT-qPCR analysis of CFTR mRNA
CFTR RT-qPCR from ALI Culture

| | | Technical | Biological | Time | Reac- |
|---|---|---|---|---|---|
| Sample | Constructs | Replicates | Replicates | Points | tions |
| CFBE41o- − RT | 4 | 1 | 3 | 3 | 36 |
| CFBE41o- + RT | 4 | 3 | 3 | 3 | 108 |

### Transepithelial Chloride Transport

CFBE4lo− cells that have been polarized under ALI conditions are transduced with AAV6-constructs, as described above. At 4 weeks post-transduction, cell inserts are mounted in an Ussing chamber (P2300, Physiologic Instruments Inc) for quantification of transepithelial Cl⁻ transport. Prior to assessment of Cl⁻ transport, a basolateral to apical chloride ion gradient is established and changes in short-circuit current (ΔIsc) are measured in the presence of 100 μM amiloride (a Na⁺ channel inhibitor) followed by sequential administration of 10 μM forskolin (a CFTR agonist) and 20 μM CFTRinh-172 (a CFTR inhibitor). CFBE4lo− cells possessing a wild-type CFTR minigene are used as a control. A summary of the analysis is shown in TABLE 26.

TABLE 26
Summary of processing strategy for protein analysis of CFTR
Transepithelial Chloride Transport from ALI Culture

| | | Technical | Biological | Time | Reac- |
|---|---|---|---|---|---|
| Sample | Constructs | Replicates | Replicates | Points | tions |
| CFBE41o- | 4 | 4 | 3 | 1 | 48 |

At the conclusion of these experiments, the lead candidate vector using ALI cultures of the CFBE4lo− cell line (F508Δ/F508Δ) is determined. These same candidate vectors are then tested in primary nasal epithelial cells derived from cystic fibrosis (CF) patients possessing different CFTR mutation profiles and unique genetic backgrounds.

Validation in Primary Nasal Epithelium from CF Patients

Final in vitro validation and characterization is conducted in primary nasal epithelial cells. While cell lines provide many practical advantages for lead development, including their capacity for indefinite expansion and amenability to both plasmid transfection experiments and virus transduction experiments, immortalization and expansion of cell lines can result in altered karyotypes and phenotypes that may not be reflective of patient-derived cells. Primary patient cells, particularly from clinically relevant tissue, therefore provide the best in vitro model for CFTR expression. Primary nasal epithelial cells can be tested naked or polarized at an air-liquid interface to maintain their hallmark phenotypes, including CFTR expression and chloride transport. Furthermore, these patient-derived cells are amenable to transduction by AAV. Patient-derived cells are used to validate the lead constructs following ALI experiments.

### Collection and Culture of Primary Cells

Nasal epithelial cells are collected via endoscopy from consenting patients following IRB-approved protocols. Primary cells are collected by brushing the mid-part of the inferior turbinate with interdental brushes. Harvested nasal epithelial cells are expanded and then cultured at an air-liquid interface, as previously described above. Briefly, nasal cells are cultured in DMEM/F-12 media in the presence of 10 μM Y-27632, a ROCK inhibitor, and irradiated fibroblast feeder cells. After 2 passages of expansion, cells are seeded onto transwell inserts and grown to confluence. Differentiation media containing Ultroser G serum substitute without reagent Y is then added for 24 hours. Cells are then maintained at an air-liquid interface by removing media from the apical compartment and providing media to the basal compartment only. The apical surface is washed with PBS to remove any mucus accumulation, and the medium is replaced in the basal compartment every 48 hours.

### AAV Transduction of Primary Nasal Cells

Transduction conditions established for ALI cultures are utilized for patient-derived cells. Cells from a single patient are divided into 6 wells of a 24-well transwell plate, with 2 wells being used for each of the two lead CFTR-AAV6 constructs and 2 wells serving as untransduced controls. CFTR-AAV6 constructs are added in duplicate to the apical surface at the optimized MOI. After 2 hours, the apical surface of cells is washed, and media is replaced. Cells are maintained until harvesting for experimental endpoints as described below. Cells from three patients, each with unique CFTR mutation profiles, are independently cultured and transduced. Replicates are used for CFTR expression and function, as further described below.

### RT-qPCR

At 4 weeks post-transduction, cells are processed for mRNA expression as described above for 2D cultures. A summary of the analysis is shown in TABLE 27.

TABLE 27
Summary of processing protocol for RT-qPCR analysis of CFTR mRNA
CFTR RT-qPCR from Primary Nasal Cells

| | | Technical | Biological | Time | Reac- |
|---|---|---|---|---|---|
| Sample | Constructs | Replicates | Replicates | Points | tions |
| −RT | 4 | 1 | 3 | 1 | 12 |
| +RT | 4 | 1 | 3 | 1 | 12 |

### Transepithelial Chloride Transport

At 4 weeks post-transduction, cell inserts are mounted in an Ussing chamber (P2300, Physiologic Instruments, Inc.) for quantification of transepithelial chloride (Cl⁻) transport, as described above. A summary of the analysis is shown in TABLE 28.

TABLE 28
Summary of Cl⁻ transepithelial transport
following transduction with CFTR AAV
Transepithelial Chloride Transport Analysis from Primary Nasal Cells

| | | Technical | Biological | Time | Reac- |
|---|---|---|---|---|---|
| Sample | Constructs | Replicates | Replicates | Points | tions |
| Nasal | 4 | 1 | 3 | 1 | 12 |
| Cells | | | | | |

### Example 4. Promoter Characterization and In Vivo Expression

This Example describes three key experiments for assessment of therapeutic potential of the CFTR AAV: (i) further characterization of the promoters in multiple lung cell lines, (ii) demonstration of promoter activity and durability in vivo, and (iii) demonstration of CFTR expression in mice.

### Promoter Characterization in Lung Cell Lines

A minimum of four technical replicates and three biological replicates per sample are used for luciferase assays, and the assay is performed in three cell lines: (i) CFBE4lo−, (ii) A549, and (iii) Calu-3. Over 75 firefly luciferase expression plasmids (including controls) have been generated. Several additional constructs are generated to provide additional benchmarks against commonly used promoters in the field, including tg83 and F5tg83. The conditions for each cell line are determined separately. This includes cell growth and plating, transfection optimization, and the firefly luciferase:NANOLUC® plasmid concentration. For example, each cell line is subcultured and seeded into 96-well plates 24 hours prior to transfection. On the day of transfection, the firefly luciferase construct is co-transfected with the NANOLUC® control construct using Lipofectamine 3000. At 24 hours post-transfection, plates are sequentially assayed for firefly luciferase and NANOLUC® using the Nano-Glo Dual-Luciferase Reporter Assay System (Promega) by imaging for total luminescence on a plate reader (Biotek); the firefly luminescence signal is normalized to the control NANOLUC® signal in each well. Technical replicates within samples are averaged together to produce a single biological replicate value. To generate a rank-ordered list for each cell line, the mean values between biological replicates are then plotted with error bars indicating the SEM. Following technical condition determination, the blinded samples are shipped and processed according to the numbers given in TABLE 29.

TABLE 29
Summary of luciferase activity following transduction with CFTR AAV
Luciferase Assay

| | | Technical | Biological | Cell | Reac- |
|---|---|---|---|---|---|
| Sample | Constructs | Replicates | Replicates | Lines | tions |
| Cell lysate | 80 | 4 | 3 | 3 | 2,880 |

### In Vivo Promoter Activity

The CFTR−/− mouse shows no spontaneous lung phenotype, and thus has limited utility as an in vivo model of CF. In addition, CFTR−/− mice exhibit severe intestinal blockage and rupture, resulting in a high rate of postnatal to post-weaning mortality. Nevertheless, there is a large gap from in vitro studies to either ferret or pig models, and therefore an in vivo proof-of-concept experiment in mice would provide a compelling rationale to pursue larger animal models. Instead of trying to rescue a pathology, such as the intestinal defects or rate of mortality observed in CFTR−/− mice, a demonstration of CFTR expression from the lead construct delivered via AAV would provide supporting in vivo data, even in the absence of a phenotypic rescue.

Due to the ubiquitous expression of the promoters, the tissues targeted are largely dependent on the AAV serotype. The AAV6 serotype has demonstrated an enhanced capacity to transduce ALI cultures of human CF bronchial airway epithelium compared to other serotypes. It has also been demonstrated in the art that the ability to efficiently deliver transgene expression to both upper and lower airways of mice and dramatically improves lung function and survival in a mouse model of Surfactant Protein B deficiency. Another important consideration for AAV6 as a vehicle for transgene delivery in the context of CF is that AAV6 carries a point mutation in its capsid protein that enables it to avoid adhesion to mucus. Thus, there is a strong rationale for using AAV6 as the delivery vehicle for a CFTR gene therapy.

In order to demonstrate promoter activity, first, in vivo luminescence driven by the candidate promoters is examined. A promoter-Luciferase reporter construct that is flanked by ITR sequences is constructed, packaged into AAV6, and delivered via intranasal administration to mice. A time course of in vivo luciferase imaging provides a direct readout of promoter activity and transgene expression in the lungs of the mice.

### Cloning and AAV6 Virus Production

Luciferase-AAV reporter constructs are generated comprising two of the lead promoters identified above, as well as a PGK1 positive control promoter. High titer and high-quality AAV vectors are generated using a plasmid transfection method. Using a single step cloning process, the transfer plasmids are constructed by excising the expression cassette used in the initial plasmid experiments with flanking NotI restriction sites, followed by ligating that cassette into a custom designed plasmid containing flanking AAV2 ITR sequences. The resulting transfer plasmids are digested by SmaI and sequenced to verify the expression cassette and ITR integrity. Validated plasmids are then amplified and maxi-prepped to generate sufficient endotoxin-free material for packaging. Packaging is done by triple-transfection of the ITR-containing transfer plasmid, a rep and serotype-specific cap plasmid, and a helper plasmid encoding adenoviral sequences. The AAV6 preps are then purified by iodixanol gradient centrifugation and concentrated. Each vector is subjected to standardized assessments, such as titering determination by droplet digital PCR, endotoxin quantification by the Limulus Amebocyte Lysate gel-clot method, and AAV prep purity and stoichiometric analysis of VP1, VP2 and VP3 capsid proteins by polyacrylamide gel electrophoresis followed by silver staining or SYPRO Red staining. Select preparations are further subjected to negative staining electron microscopy, which enables determination of empty to full vector particles.

Prior to study onset, 48 wild-type C57BL/6 mice are randomized into four groups (six males and six females per group); two groups comprising the two lead constructs and two groups comprising controls. AAV transduction in the mouse is known to be sexually dimorphic, with greater expression in males along with altered tissue distribution in organs, such as the lung. Accordingly, groups of mice representing equal numbers from both sexes are tested, and data is plotted according to sex. At 8 weeks of age, each group receives a single 50 μl intranasal instillation of either 2×1014 VG/kg AAV6 or sterile PBS. Briefly, intranasal delivery is conducted on anesthetized mice that have been positioned upright with their necks straight and their mouths closed. Small volumes of the test material are pipetted onto the bridge of the nose in a manner that enables the nares to inhale the liquid in an alternating fashion until all of the test material has been aspirated.

### In Vivo Luciferase Activity

Mice are followed for a total of 32 weeks post-infection (for 14 time points) to comprehensively assess peak luciferase expression and vector durability. Mice are injected intraperitoneally with 75 mg/kg D-luciferin in 100 μL of PBS and placed in the chamber of the imaging system under isoflurane anesthesia. 10 minutes post-injection, luminescent images are acquired (Xenogen IVIS). In vivo luciferase expression enables following the kinetics of expression onset along with quantification of promoter activity without having to sacrifice mice. A control vector driving luciferase expression from the PGK1 promoter is used to compare tissue distribution and expression level. Tissue distribution is examined over time to confirm that expression is not silenced as compared with the PGK1 promoter. Imaging is conducted weekly over the first 8 weeks, followed by imaging at 4-week intervals until up to 32 weeks after vector delivery. A summary of the in vivo experiments is shown in TABLE 30.

TABLE 30
Summary of in vivo luciferase activity
following transduction with CFTR AAV

| | | | | | |
|---|---|---|---|---|---|
| | | Male Mice | Female Mice | Time | Reac- |
| Sample | Groups | per group | per group | Points | tions |

Short-Term in vivo Bioluminescence (1-8 weeks)

| | | | | | |
|---|---|---|---|---|---|
| Mouse | 4 | 6 | 6 | 8 | 384 |
| Lung | | | | | |

Long-Term in vivo Bioluminescence (12-32 weeks)

| | | | | | |
|---|---|---|---|---|---|
| Mouse | 4 | 6 | 6 | 6 | 288 |
| Lung | | | | | |

It is hypothesized that in order to achieve durable expression, the AAV6 vectors must target dividing cell types (e.g., basal stem cells) and the promoters need to evade the silencing or toxicity that is typical of many viral promoters.

### In Vivo AAV6-CFTR Expression

In vivo CFTR expression in mice is demonstrated. Data collected heretofore include in vitro promoter activity, in vitro CFTR expression, in vitro AAV transduction to ALI cultures and patient samples, in vitro CFTR functional rescue, and in vivo promoter activity have been demonstrated.

Prior to study onset, 24 wild-type C57BL/6 mice are randomized into two groups (12 males and 12 females per group), comprising 1 lead construct and a control group. At 8 weeks of age, each group receives a single 50 μl intranasal instillation of either 2×10¹⁴ VG/kg AAV6 or sterile PBS, as described above. Mice (3 males and 3 females per group) are sacrificed at 4- and 8-weeks post-injection. Lungs are perfused through the right ventricle with PBS to remove blood, followed by collection of lung tissue. Lungs are harvested for RT-qPCR and western blot analyses.

### RT-qPCR

RNA is isolated from lung tissue (using an RNeasy Mini Kit, Qiagen kit, or equivalent). cDNA is generated (High-Capacity cDNA Reverse Transcription Kit, ThermoFisher), followed by expression analysis using qPCR assays specific for hCFTR (transgenic expression), mCFTR (endogenous expression), and mGAPDH. Data are aggregated and plotted according to sex of the mouse, as shown in TABLE 31.

TABLE 31
Summary of processing protocol for RT-qPCR analysis of CFTR mRNA
in vivo CFTR RT-qPCR

| | | Male Mice | Female Mice | Time | Reac- |
|---|---|---|---|---|---|
| Sample | Groups | per group | per group | Points | tions |
| Mouse | 2 | 3 | 3 | 3 | 24 |
| Lung | | | | | |

### Western Blot

Protein is isolated from lung tissue using RIPA buffer plus protease inhibitors. Tissues are homogenized, and protein is separated from tissue debris via centrifugation. 10-30 μg total protein are analyzed via western blot, as described for the in vitro assays above, and as shown in TABLE 32 below.

TABLE 32
Summary of processing protocol for Western Blot
analysis of CFTR protein
in vivo CFTR Western Blot Analysis

| | | Male Mice | Female Mice | Time | Reac- |
|---|---|---|---|---|---|
| Sample | Groups | per group | per group | Points | tions |
| Mouse | 2 | 3 | 3 | 3 | 24 |
| Lung | | | | | |

Following the above experiments, in vivo AAV6-CFTR expression from the lead candidate vectors is confirmed. Importantly, this would be the only full-length CFTR AAV therapeutic in development, enabled by the compact promoters discovered as described above.

Building upon the in vivo data in mice, future experiments will be expression and phenotypic rescue in larger animals (ferrets or pigs), and demonstration of chloride channel function in the nasal epithelia of cystic fibrosis patients in the clinic.

### Example 4: Design of Codon Optimized CFTR Sequences

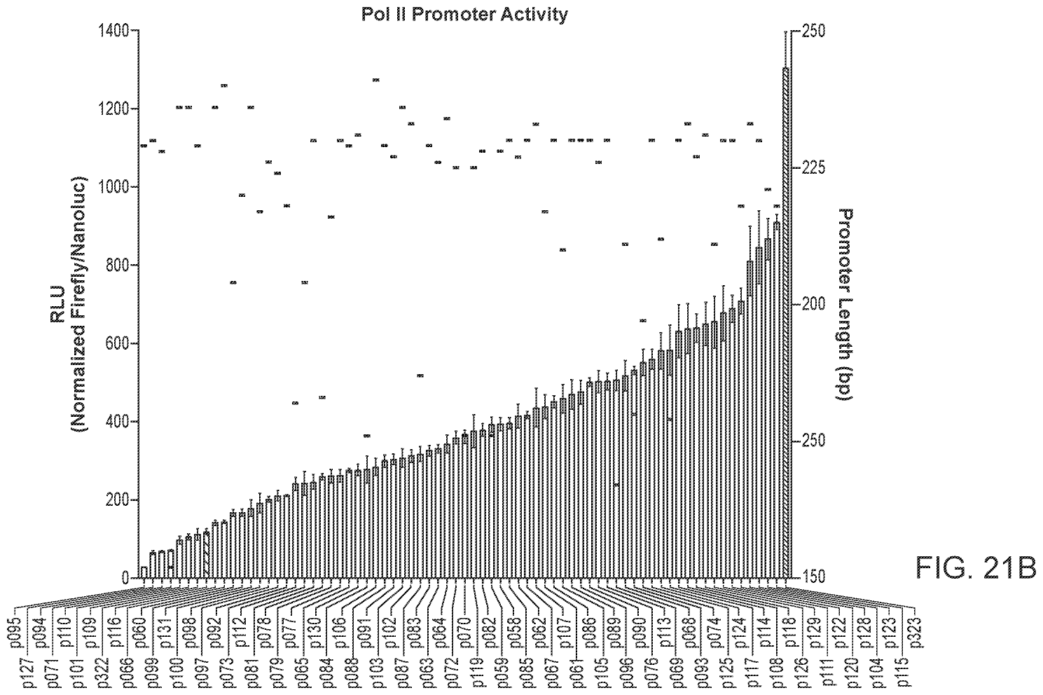

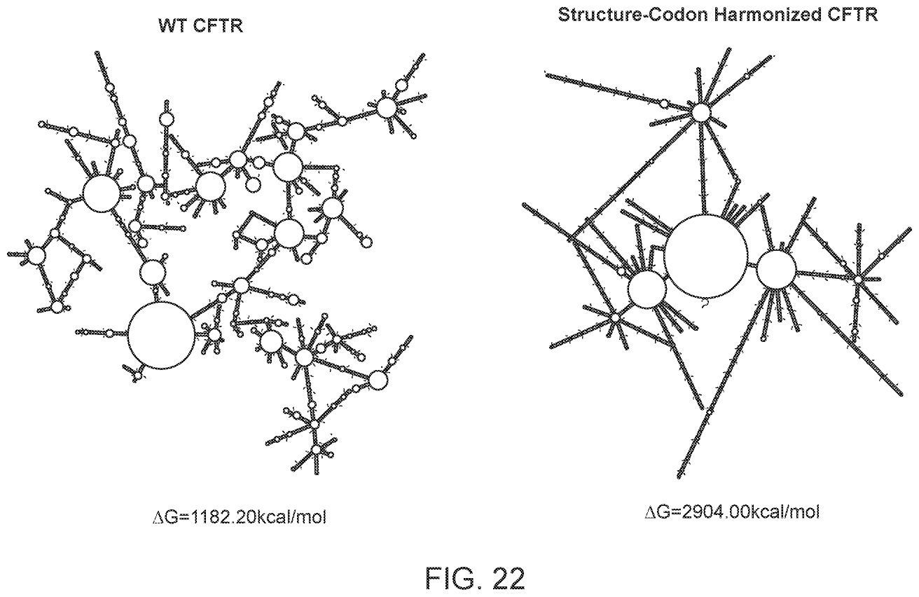

Using an RNA structure-based approach, the secondary structure for different CFTR encoding mRNAs is predicted, and stability is assessed based on calculated thermodynamic values. Wild-type CFTR cDNA sequence was found to fold into a structure with significant unpaired nucleotides having a ΔG=−1182.20 kcal/mol. In one example of a structure-codon optimized CFTR sequence, base-pairing was significantly increased and the stability of the mRNA structure was also increased (ΔG=−2904.00 kcal/mol) (FIG. 22).

### Structure-Codon Optimization.

Based on the outlined criteria, 10 CFTR sequences are optimized based on an iterative RNA-folding and codon optimization/harmonization process, generating sequences representing a range of thermodynamic stability. Strong base-pairing among the first 10 residues are avoided as this is known to affect translation. (Mauger et al. (2009) “mRNA structure regulates protein expression through changes in functional half-life,” Proc Natl Acad Sci USA 116:24075-24083.) Model unstructured UTRs (human β-globin 5′UTR and rabbit β-globin 3′UTR) flank the coding sequence (CDS). Optimized CFTR sequences have the human β-globin 5′UTR and rabbit β-globin 3′UTR appended to the mRNA sequence and computationally refolded to confirm the absence of extensive base-pairing occurring between each optimized sequence and the model UTRs.

### Codon Optimization/Harmonization.

The coding sequence is optimized for a variety of factors known to affect gene expression and viral gene delivery: codon usage bias, GC content, CpG dinucleotides, mRNA secondary structure, cryptic splicing sites, premature poly(A) sites, RNA instability motifs, AT-rich elements (ARE), and repeat sequences (direct repeat, reverse repeat, and dyad repeat). Codon usage bias are determined using the codon adaptive index (CAI), favoring scores >0.70, and the frequency of optimal codons (FOP) is targeted above 80%. GC content is targeted to between 30-70%, and unfavorable GC peaks are optimized to prolong the half-life of the mRNA. Cis-acting elements, including splice donors/acceptors (GGTAAG, GGTGAT, GTAAAA, GTAAGT), PolyA (AATAAA, ATTAAA, AAAAAAA), destabilizing motifs (ATTTA), AT-rich elements (ATTTTA, ATTTTTA, ATTTTTTA), PolyT (TTTTTT), polymerase slippage sites (GGGGGG, CCCCCC), and internal Kozak sequences (ACCACCATGG, GCCACCATGG) are avoided. Additionally, vectors are analyzed for the presence of stem-loop structures that impact ribosomal binding and stability of mRNA, and antiviral motifs (TGTGT, AACGTT, CGTTCG, AGCGCT, GACGTC, GACGTT) are modified.

### Constructs and Cloning.

Eight to sixteen constructs including the WT CDS are synthesized (Twist Biosciences) and cloned downstream of a bidirectional promoter and flanked between a human β-globin 5′UTR and rabbit β-globin 3′UTR. These elements are fixed between all tested constructs and the rabbit β-globin 3′UTR is used for RT-PCR analysis. Constructs are maxi-prepped and purified under endotoxin-free conditions.

### RT-qPCR

In order to assess the effects of optimization on mRNA stability, the half-life of CFTR is determined by RT-qPCR following actinomycin treatment. This assay is performed in A549 cells, which do not endogenously express CFTR. A single primer pair and probes against the rabbit β-globin 3′UTR are designed to standardize detection between all constructs (FIG. 20). RT priming occurs via an oligo(dT) primer, and qPCR detection in the 3′UTR region is designed to avoid potential issues arising from extensive RNA secondary structures within the coding sequences. Cells are sub-cultured for 3-5 passages, then seeded into 96-well plates. At 24h after seeding, cells are transfected with plasmids using Lipofectamine 3000. At 48h post transfection, cells are treated with 2 μM actinomycin D or DMSO at 0, 3, and 6 hours to arrest transcription. At each time point, cells are lysed in QuickExtract RNA and treated with DNase I according to manufacturer's protocol (Lucigen). Cells are treated again with DNase I (Turbo DNase I, Invitrogen) to remove any traces of contaminating DNA, and then purified by column (Zymo RNA Clean & Concentrator). RNA is quantitated and 20 ng-2 μg per sample are used for cDNA synthesis with a poly(T) reverse primer; all reactions include a no RT (−RT) control (High-Capacity cDNA Reverse Transcription Kit, ThermoFisher). cDNA reactions and no RT controls are then used as templates for qPCR reactions containing rabbit β-globin 3′UTR primer-pair probes (custom assays from ThermoFisher or IDT) and control human GAPDH predesigned primer-pair TaqMan assays (ThermoFisher) with a 2× TaqMan Multiplex Master Mix (ThermoFisher). RT-qPCR analysis is only valid from samples without amplification in −RT reactions. Expression and stability of each variant is compared to the wild-type CFTR CDS. Following SOP determination, which includes optimization of singleplex and multiplex TaqMan assays, the blinded samples are shipped and processed according to these numbers:

TABLE 33
Summary of processing protocol for RT-qPCR analysis of CFTR mRNA
CFTR RT-qPCR in A549 Cells

| | | Technical | Biological | Time | Reac- |
|---|---|---|---|---|---|
| Sample | Constructs | Replicates | Replicates | Points | tions |
| −RT | 8 | 1 | 3 | 1 | 24 |
| +RT | 8 | 3 | 3 | 3 | 216 |

After analyzing the data from these experiments, constructs may be iterated and optimized. For example, the process is repeated for a total of 16 constructs. In addition to transfecting 8 plasmid DNA constructs into cells, T7 in vitro-transcribed mRNA sequences corresponding to each DNA construct will be transfected. Direct transfection of mRNA will be used to determine mRNA stability and translation isolated from plasmid transcription.

### Western Blot.

To determine the protein levels of WT CFTR, western blot analysis is performed using well-established methods. At 48h post-transfection, cells are lysed in RIPA buffer containing protease inhibitors. 10-30 μg of lysate are run on SDS-PAGE gels (Invitrogen), transferred to PVDF membranes, and probed with antibodies against CFTR (ab596, CFF Antibody Distribution Program) and β-actin. Western blot bands are quantified, noting B band and C band corresponding to the core glycosylated and mature glycosylated forms of CFTR, respectively. In order to reduce the total number of samples, 5 constructs are chosen including the two controls for protein analysis and one cell line will be analyzed.

TABLE 34
Summary of processing protocol for RT-qPCR analysis of CFTR mRNA
CFTR Western Blot Analysis

| | | Technical | Biological | Time | Reac- |
|---|---|---|---|---|---|
| Sample | Constructs | Replicates | Replicates | Points | tions |
| Protein | 8 | 3 | 3 | 1 | 72 |

Secondary structures could have negative effects on AAV packaging. (Xie, J. et al. (2017) “Short DNA Hairpins Compromise Recombinant Adeno-Associated Virus Genome Homogeneity,” Mol Ther 25:1363-1374.) To rule this out, constructs are cloned into ITR-containing plasmids and small-scale packaging are used to verify that there are no adverse effects. Small scale preps are produced. HEK293T cells are used in triple-transfected reactions, with an ITR-containing transfer plasmid, a rep and serotype-specific cap plasmid, and a helper plasmid encoding adenoviral sequences. 72 hours following transfection, cells are collected and subjected to three freeze-thaw cycles to release viral particles. Following centrifugation to remove cell debris, the supernatant is stored at −80° C. Crude lysate is used to transduce HEK293 cells and RT-qPCR or protein capillary electrophoresis is used to validate expression.

The promoters, codon optimized CFTR, and terminator are selected based on multiple factors, including construct size and expression level; 3-4 candidates are selected. These candidates are assembled from the individual candidate elements identified above, then are synthesized and cloned as they will exist in their final expression cassettes.

It is expected that this method will identify optimized individual components for subsequent assembly into an AAV size-suitable construct for validation experiments.

### Example 5. Mouse H1 Promoter Deletion Analysis

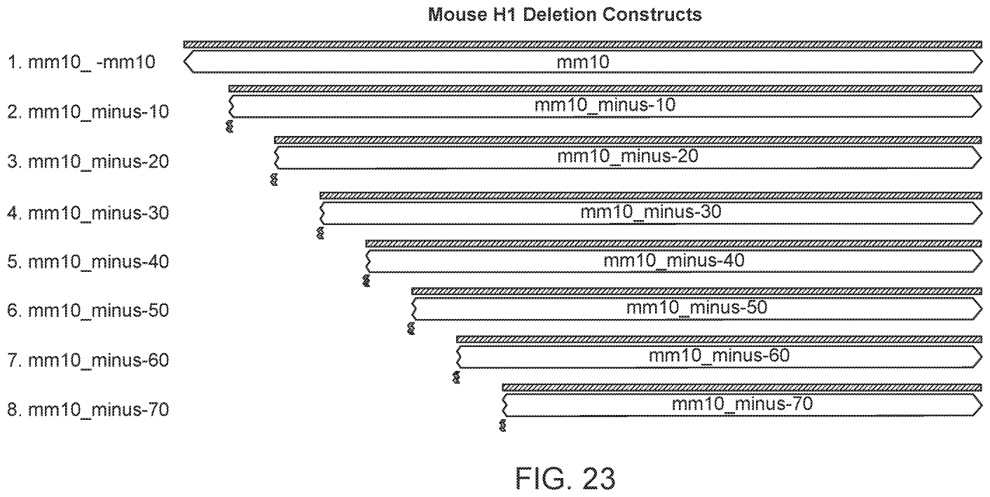

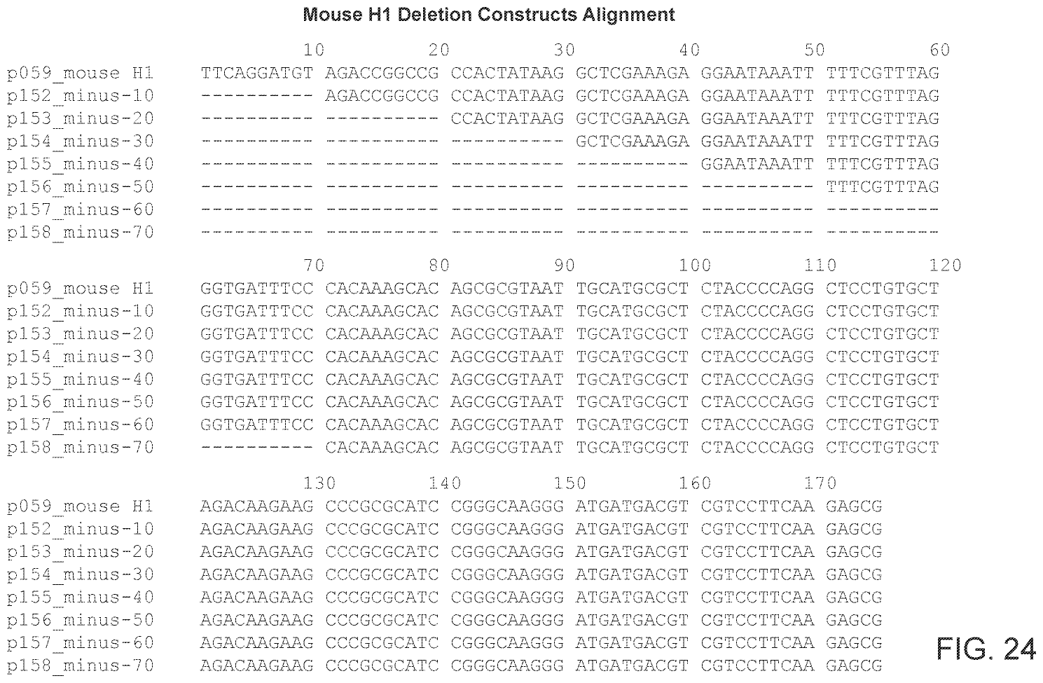

To determine which regions of the mouse H1 promoter were needed for activity, a series of mouse H1 promoter constructs were made and tested. A schematic representation of the mouse H1 promoter deletion constructs is shown in FIG. 23, with the wild-type mouse promoter (p059, SEQ ID NO: 93) shown at the top and seven successive 10 bp deletion constructs shown below. An alignment of the various deletion constructs is provided in FIG. 24. These promoters and variants were used to drive reporters and quantitate expression.

To test the relative activity of promoters, luciferase reporter constructs were designed that enable quantitation of the Pol II promoter activity of the promoters. To reduce confounding noise and spurious reporter gene transcription, the plasmid constructs contain 5′ and 3′ beta-globin insulators that flank the expression cassette; the promoter sequence connected to a control guide RNA on one side and firefly luciferase on the other side, and bGH poly(A) signal are found inside the insulators.

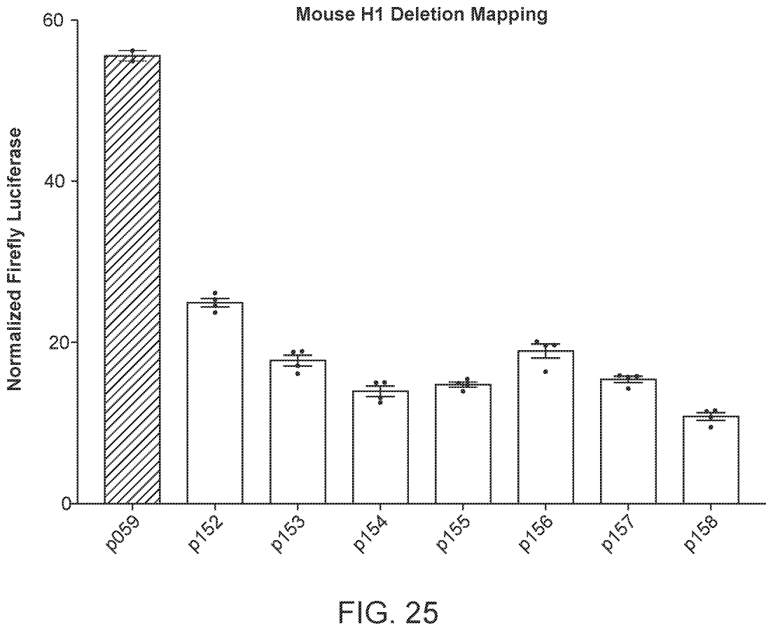

Generally, cell lines were subcultured and seeded into 96-well plates 24 hours prior to transfection. On the day of transfection, the firefly luciferase construct was co-transfected with the NANOLUC® control construct using Lipofectamine 3000. At 24 hours post-transfection, plates were sequentially assayed for firefly luciferase and NANOLUC® using the Nano-Glo Dual-Luciferase Reporter Assay System (Promega) by imaging for total luminescence on a plate reader (Biotek). For data analysis and plotting, the firefly luminescence signal was normalized to the control NANOLUC® signal in each well. Technical replicates within samples were averaged together to produce a single biological replicate value, and the mean values between biological replicates were then plotted with error bars indicating the SEM. Results are shown in FIG. 25 (normalized firefly to NANOLUC® luciferase signal for each construct).

As shown in FIG. 25, each deletion construct retained a portion of the full-length wild-type H1 promoter activity. It is contemplated that fragments of H1 promoters (e.g., the H1 promoters described herein) that retain activity can be used to express large genes. For example, expression levels of a large gene produced by using a fragment of an H1 promoter may be sufficient, in certain circumstances, to improve symptoms of a disease, such as cystic fibrosis, Wilson disease, Menkes disease, Cori Disease, Duchenne Muscular Dystrophy, or carbamoyl phosphate synthetase I deficiency (CPS1D)).

### Example 6. Mouse H1 Promoter Mutation Analysis

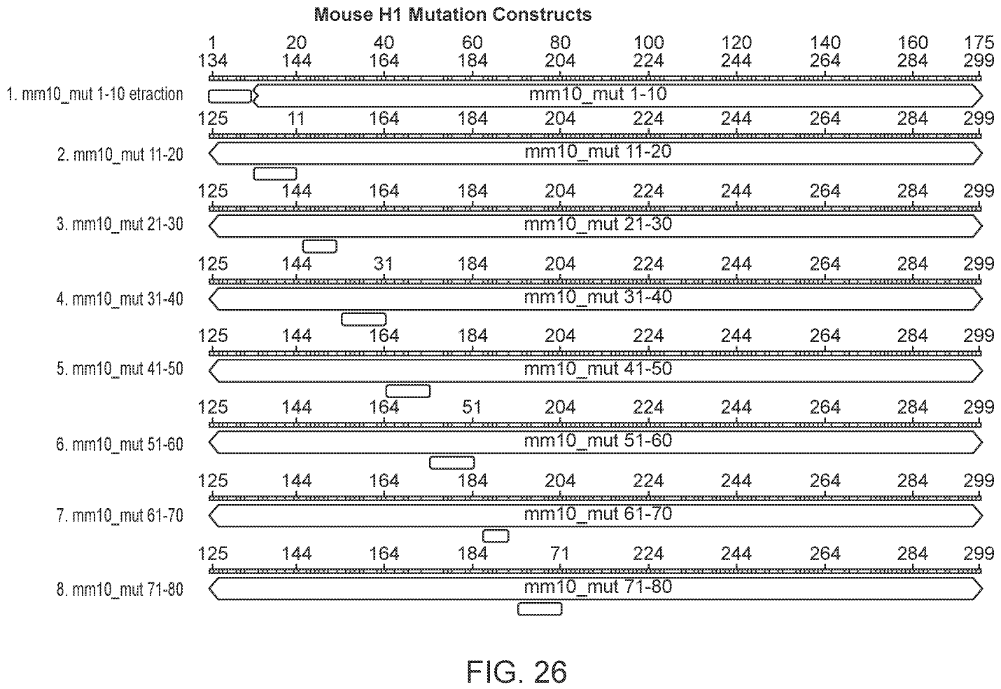

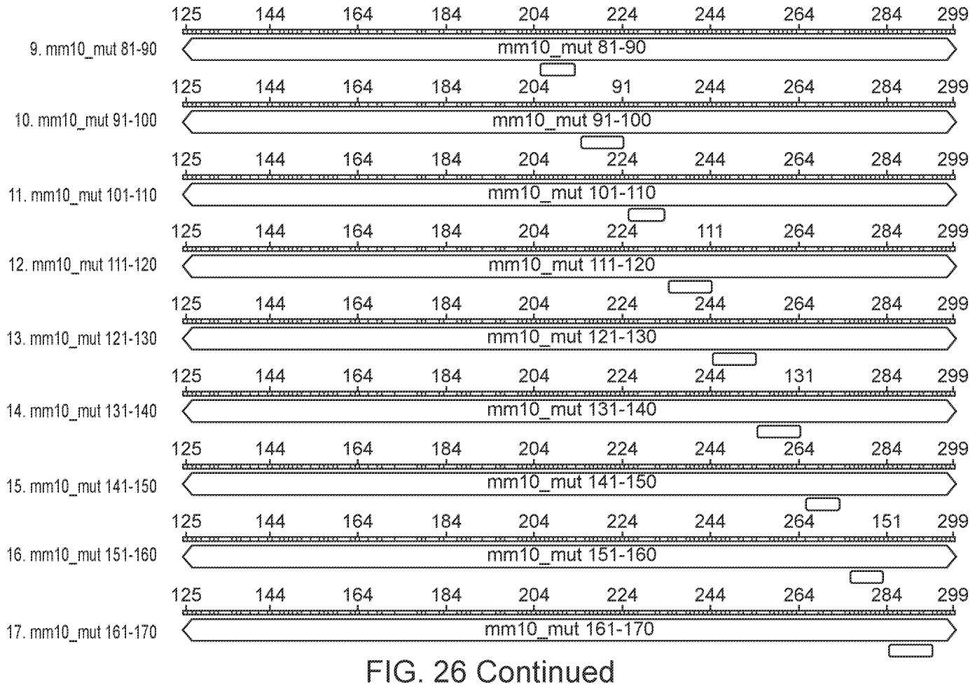

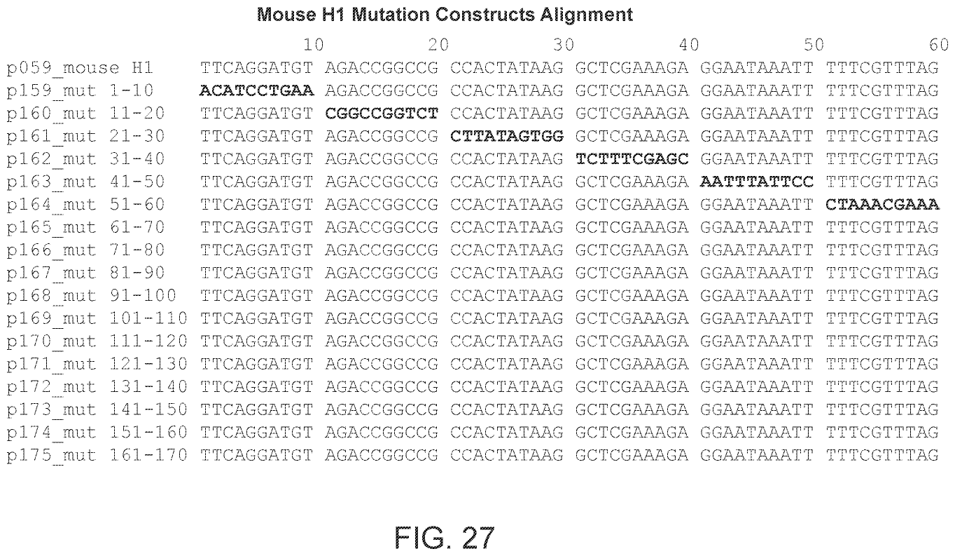

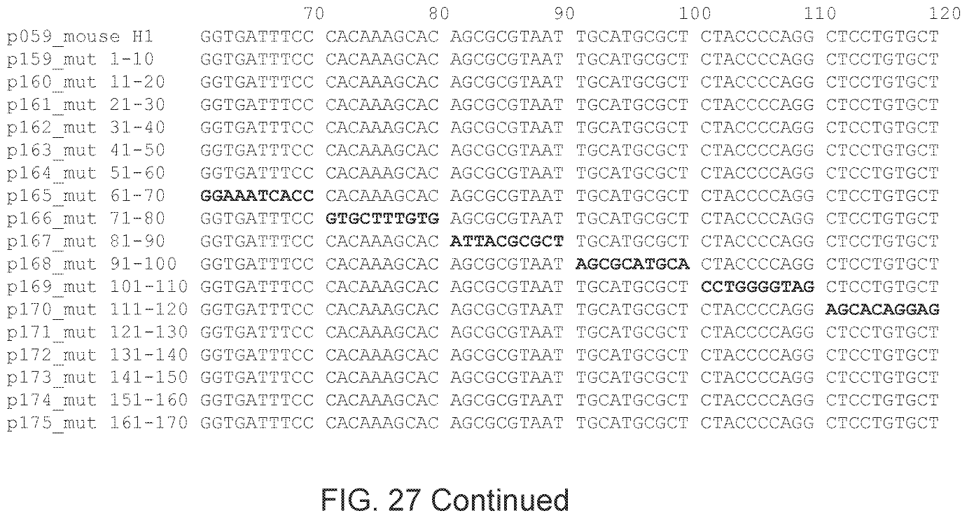

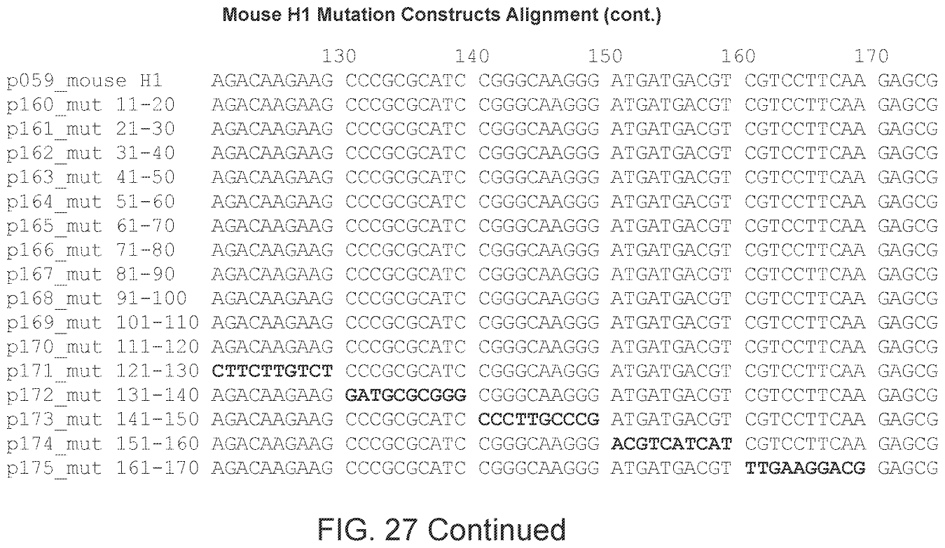

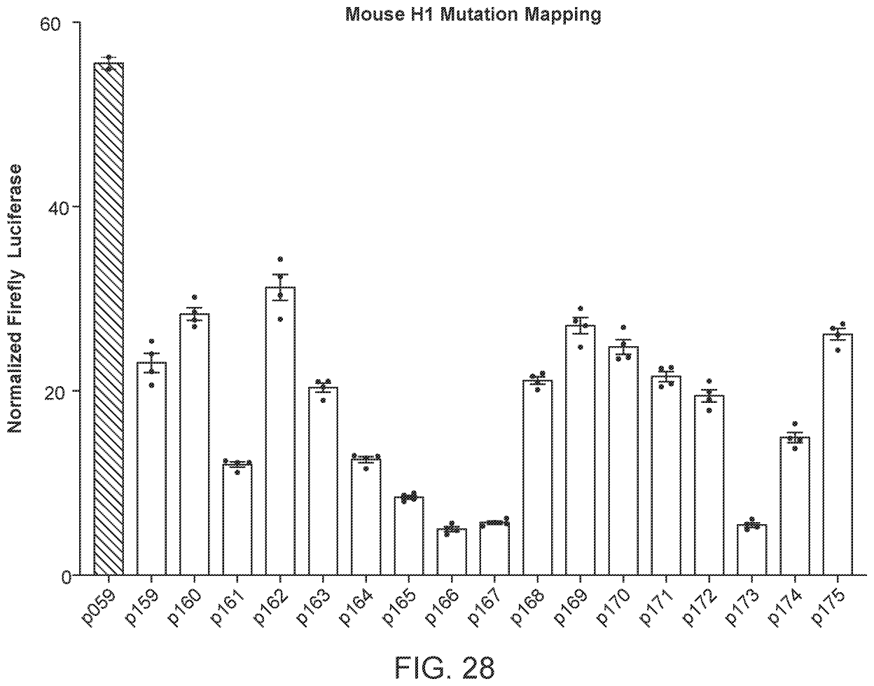

Seventeen (17) mutation constructs were designed by walking across the promoter in 10 bp increments and replacing the sequence with its reverse complement. A schematic representation of the constructs is shown in FIG. 26 and an alignment of the sequences shown in FIG. 27. Constructs were made and tested as described in Example 5. Results are shown in FIG. 28.

As shown in FIG. 28, each mutation construct retained a portion of the full-length wild-type H1 promoter activity. It is contemplated that variants of H1 promoters (e.g., the H1 promoters described herein) that retain activity can be used to express large genes. For example, expression levels of a large gene produced by using a variant of an H1 promoter may be sufficient, in certain circumstances, to improve symptoms of a disease, such as cystic fibrosis, Wilson disease, Menkes disease, Cori Disease, Duchenne Muscular Dystrophy, or carbamoyl phosphate synthetase I deficiency (CPS1D)).

### Example 7. Mouse H1 Promoter with Introns

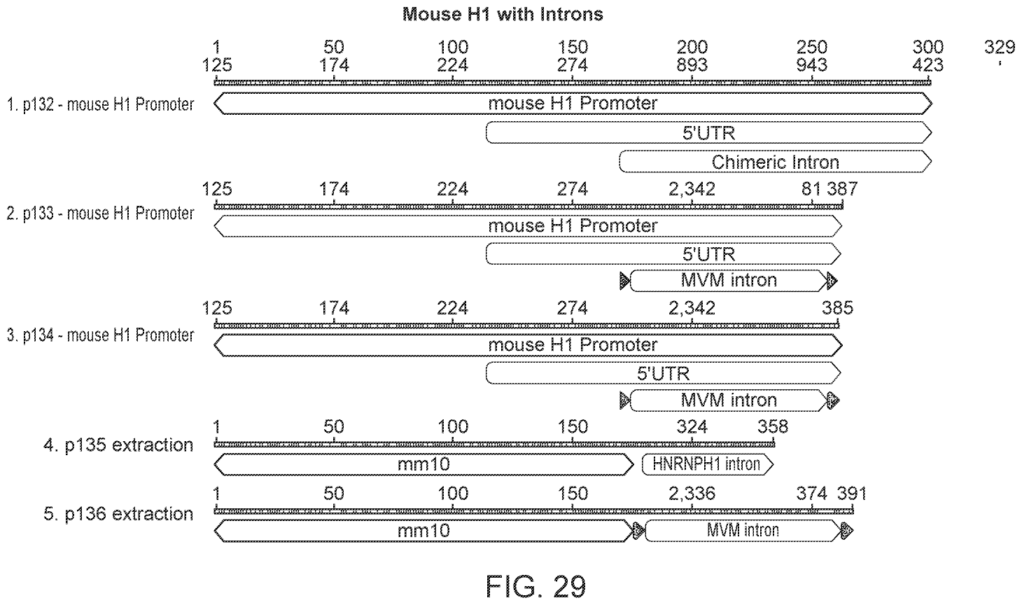

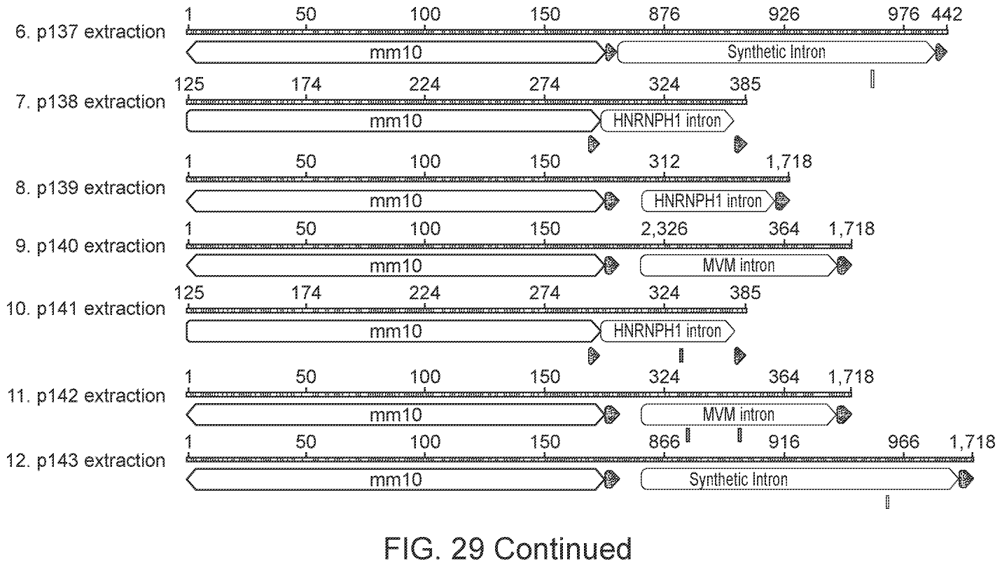

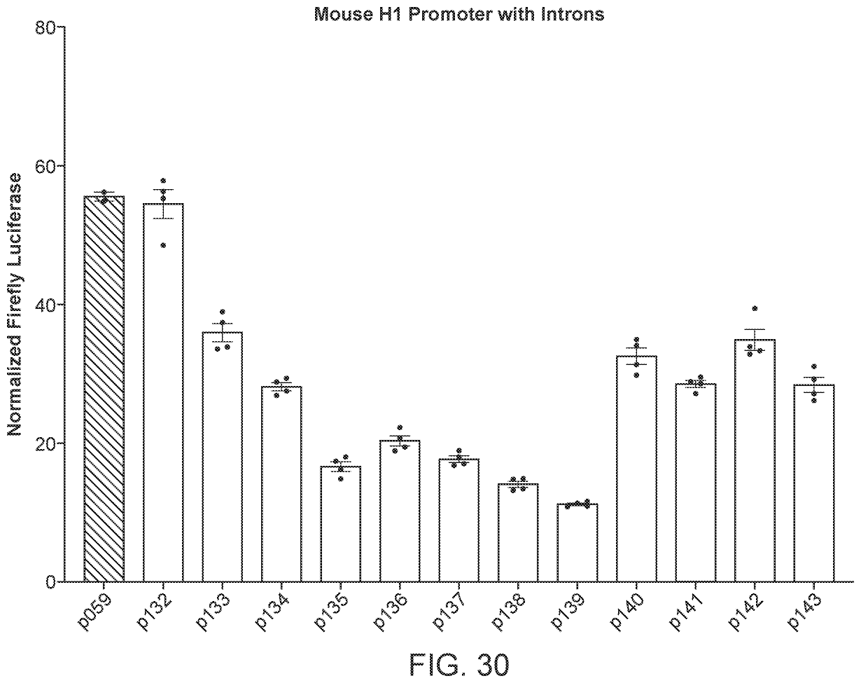

Twelve (12) different constructs were designed to incorporate introns into the mouse H1 promoter region. Different intron sequences and different insertion locations were used as shown in FIG. 29. Constructs were made and tested as described in Example 5. Results are shown in FIG. 30.

As shown in FIG. 30, each intron construct retained at least a portion of the full-length wild-type H1 promoter activity. It is contemplated that variants (e.g., intron-containing variants) of H1 promoters (e.g., the H1 promoters described herein) that retain activity can be used to express large genes. For example, expression levels of a large gene produced by using a variant of an H1 promoter may be sufficient, in certain circumstances, to improve symptoms of a disease, such as cystic fibrosis, Wilson disease, Menkes disease, Cori Disease, Duchenne Muscular Dystrophy, or carbamoyl phosphate synthetase I deficiency (CPS1D)).

### Example 8. Human and Mouse H1 5′UTR Constructs

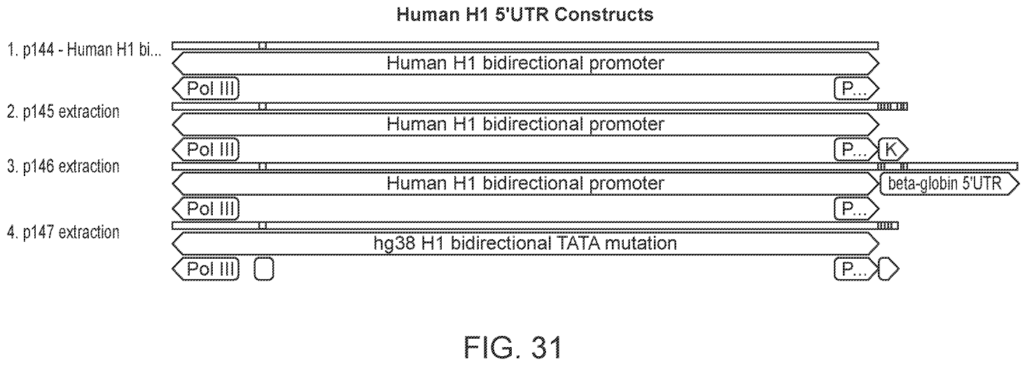

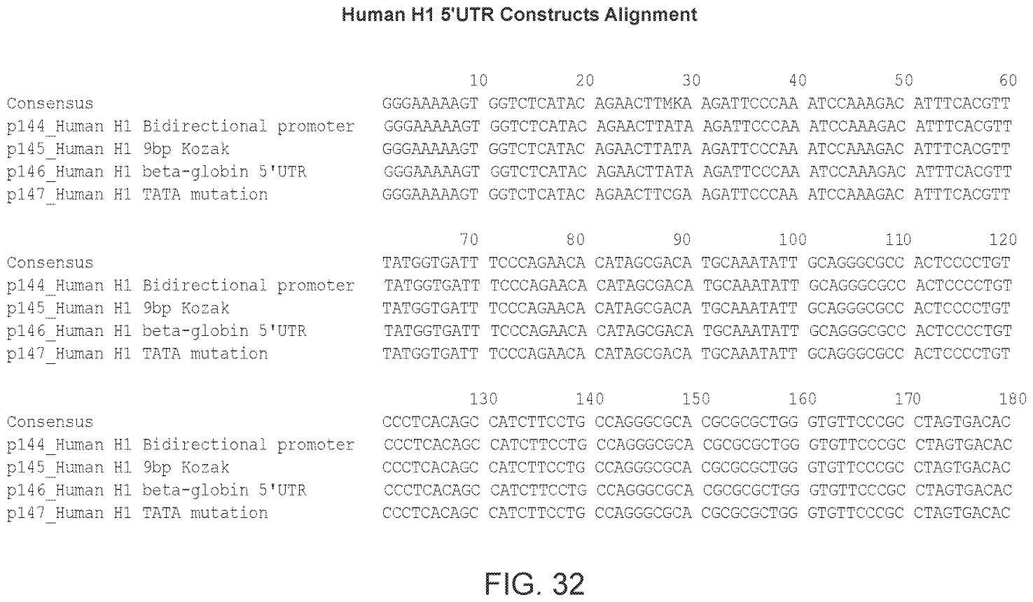

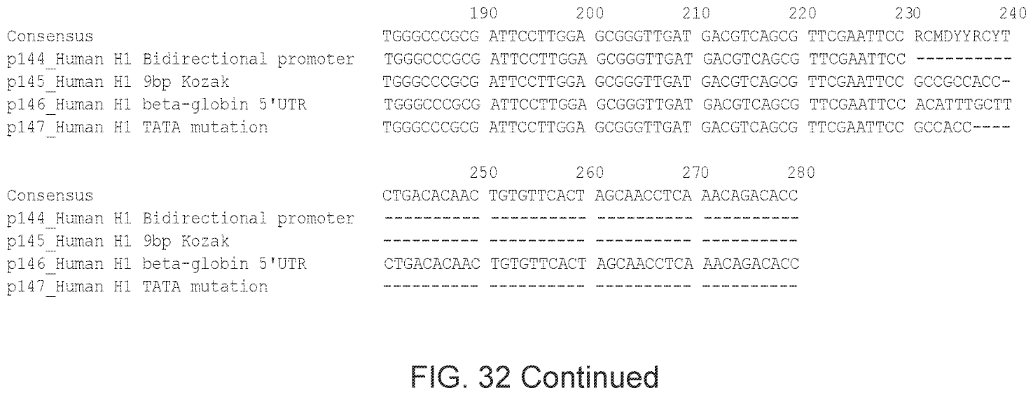

FIG. 31 provides a schematic showing the design of human H1 promoter and variant constructs. As shown in FIG. 31, a construct carrying a human H1 promoter alone, a human H1 promoter with a 9 bp Kozak sequence (GCCGCCACC (SEQ ID NO: 256)), a human H1 promoter with a beta-globin 5′UTR, and a human H1 promoter with a TATA box mutation (TATAA→TCGAA) were designed. An alignment of the sequences is shown in FIG. 32.

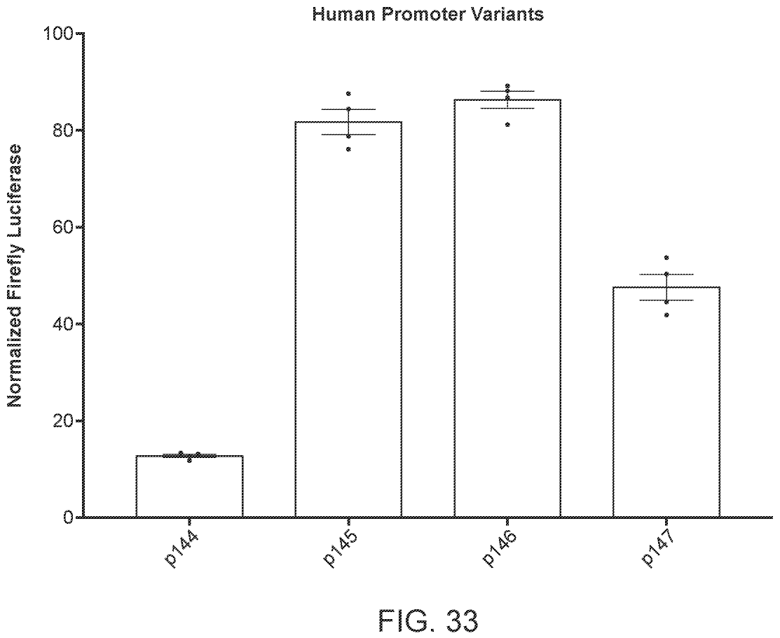

Constructs were made and tested as described in Example 5. Results are shown in FIG. 33.

As shown in FIG. 33, addition of 5′UTR sequences increased expression from an H1 promoter. Accordingly, such 5′UTR sequences can be used to increase expression from a promoter as described herein (e.g., an H1 promoter).

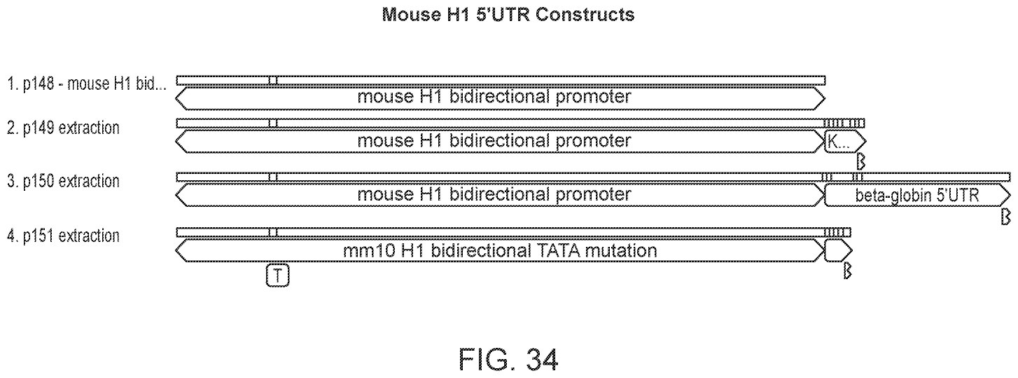

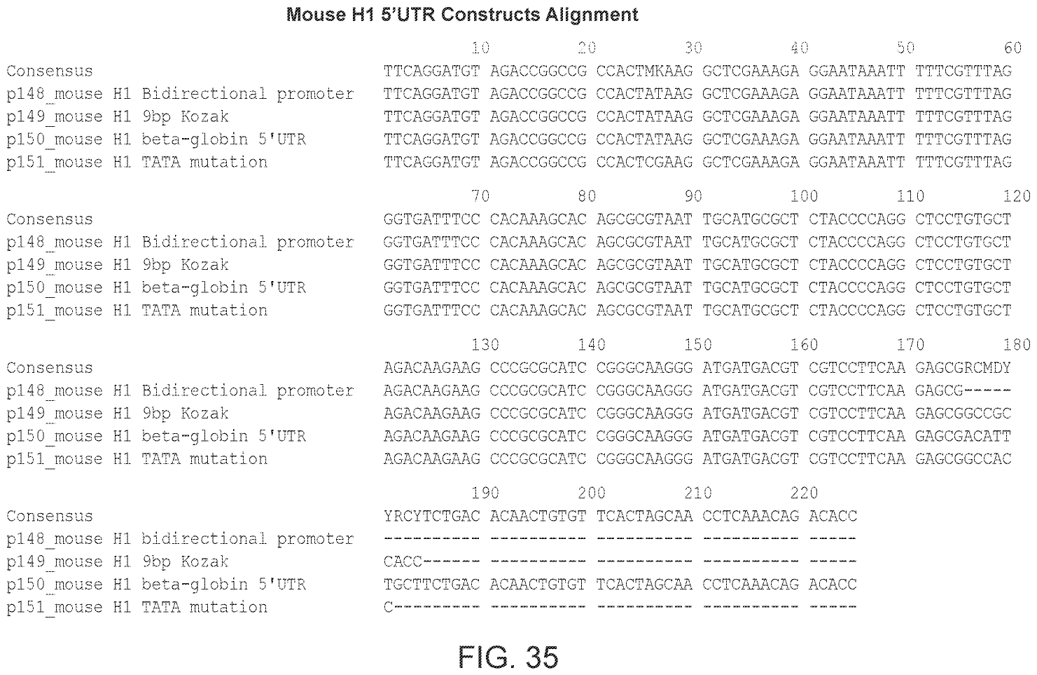

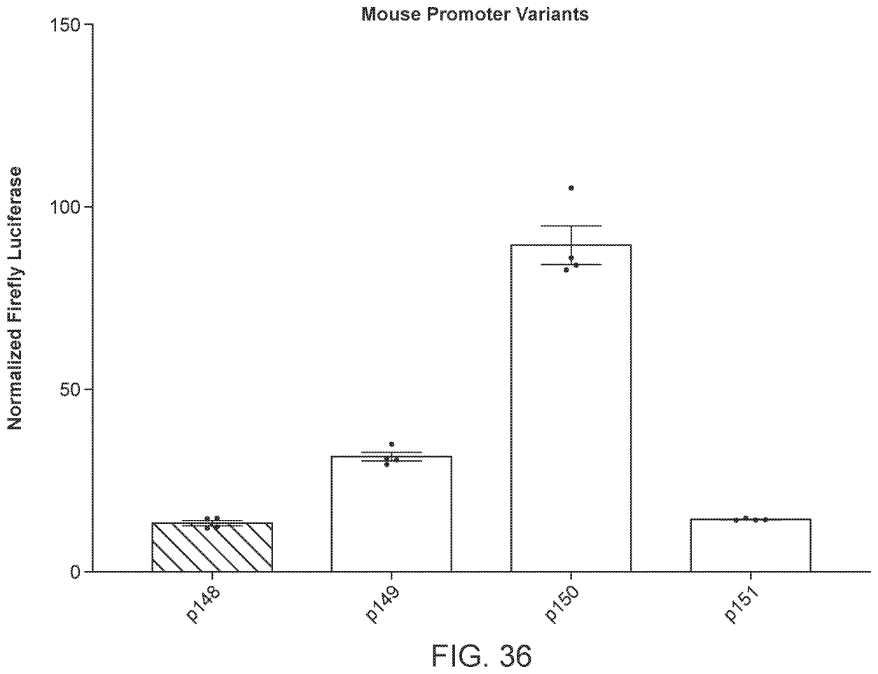

H1 5′UTR constructs also were made and tested using the mouse H1 promoter, as shown in FIGS. 34 and 35. Results are shown in FIG. 36.

As shown in FIG. 36, most of the tested 5′UTR sequences increased expression from a mouse H1 promoter. Accordingly, such 5′UTR sequences can be used to increase expression from a promoter as described herein (e.g., a mouse H1 promoter).

### Example 9. Expression of H1, Gar-1 and Other Bidirectional Promoters

Additional constructs were designed as described above, but using the following promoters: human H1 (p144; SEQ ID NO: 87), mouse H1 (p148; SEQ ID NO: 93), human 7sk-1 (p199; SEQ ID NO: 242), mouse 7sk-1 (p203; SEQ ID NO: 204), human ALOXE3 (p204; SEQ ID NO: 246), human CGB1 (p206; SEQ ID NO: 247), human CGB2 (p207; SEQ ID NO: 248), human GAR1-1 (p216; SEQ ID NO: 107), human Medi6-1 (p222; SEQ ID NO: 249), human Medi6-2 (p223; SEQ ID NO: 250), human SRP (p242; SEQ ID NO: 233).

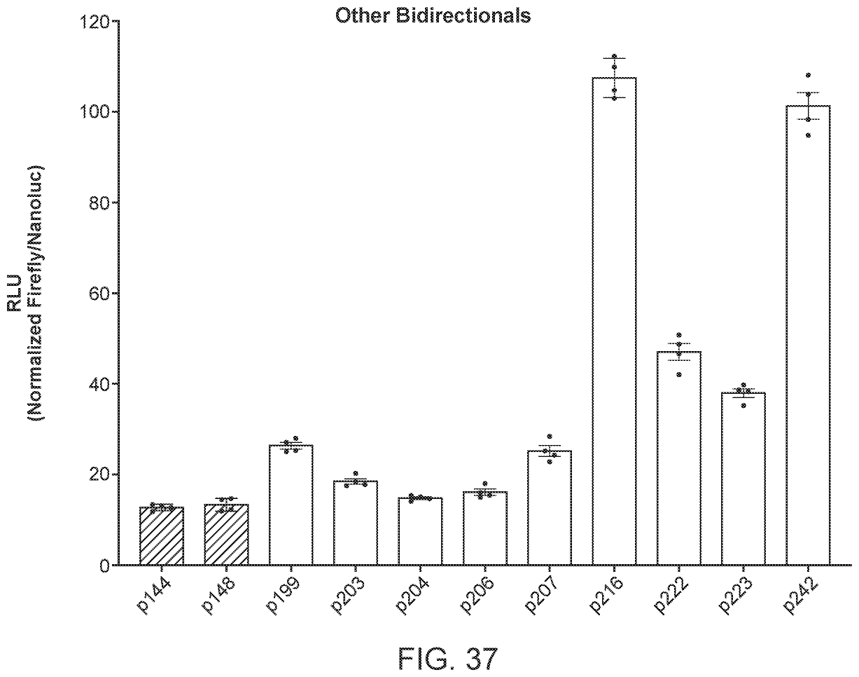

Constructs were made and tested as described above. Results are shown in FIG. 37.

As shown in FIG. 37, most of the tested bidirectional promoters showed increased expression as compared to an H1 promoter. Gar-1 showed the highest level of expression. Accordingly, such compact bidirectional promoters can be used to express a large gene using a vector, such as an AAV vector, that has limited space.

### Example 10. Assessment of Promoter Activity in CF Relevant Cell Lines

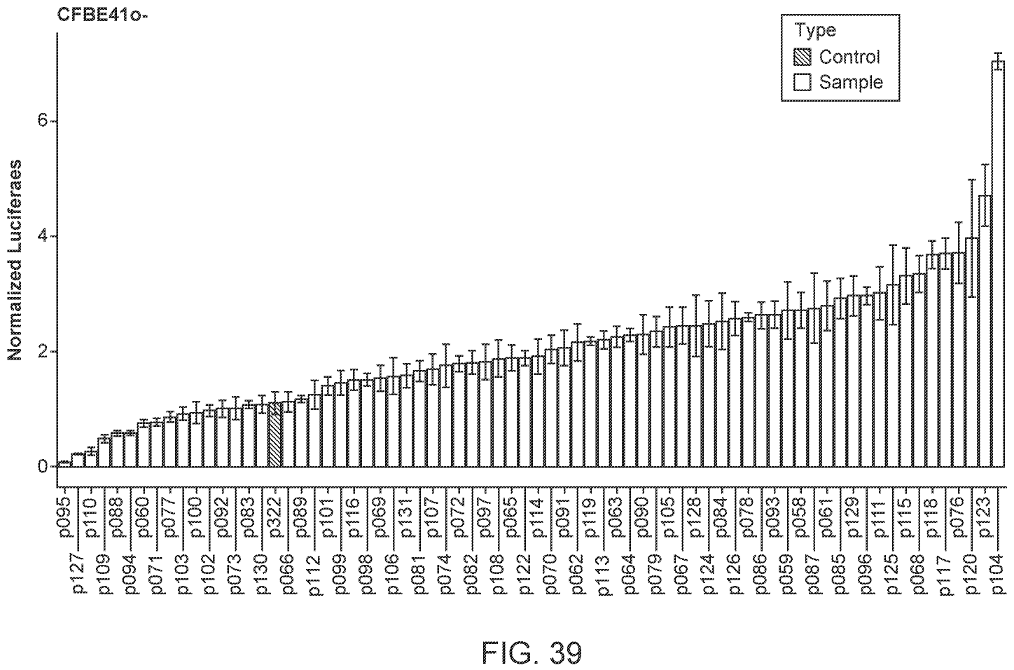

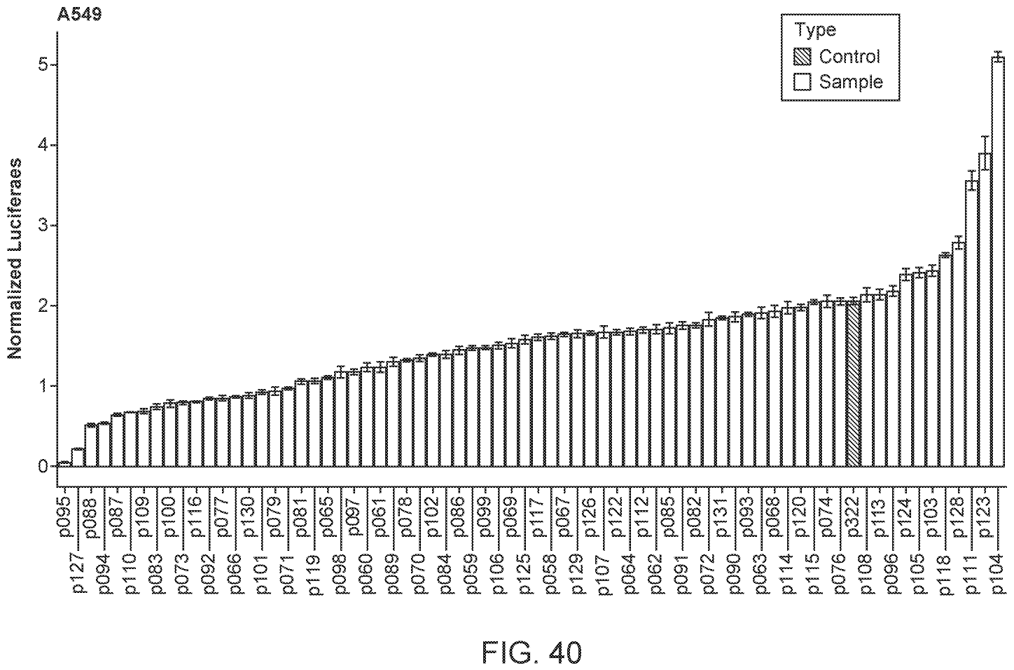

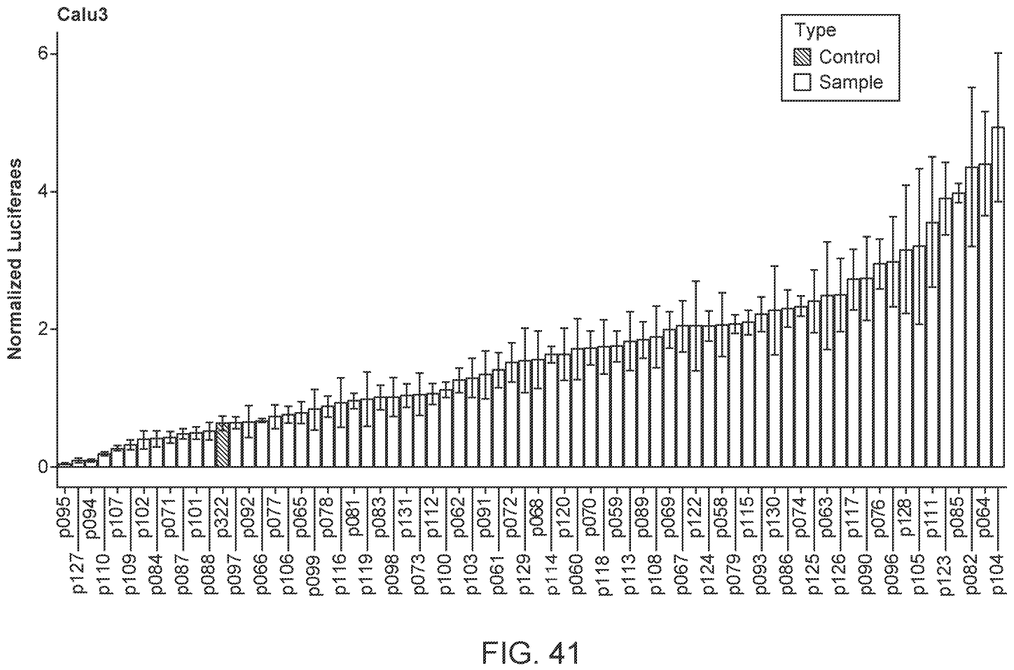

This Example describes the characterization of a library of H1 promoters for their capacity to drive gene expression using luciferase reporters (Firefly luciferase and NANOLUC®) in three lung cell lines (A549, Calu-3, and CFBE4lo−). Normalized luciferase expression was quantified for 71 H1 promoters and benchmarked against a control thymidine kinase (TK) promoter (FIGS. 39, 40, and 41).

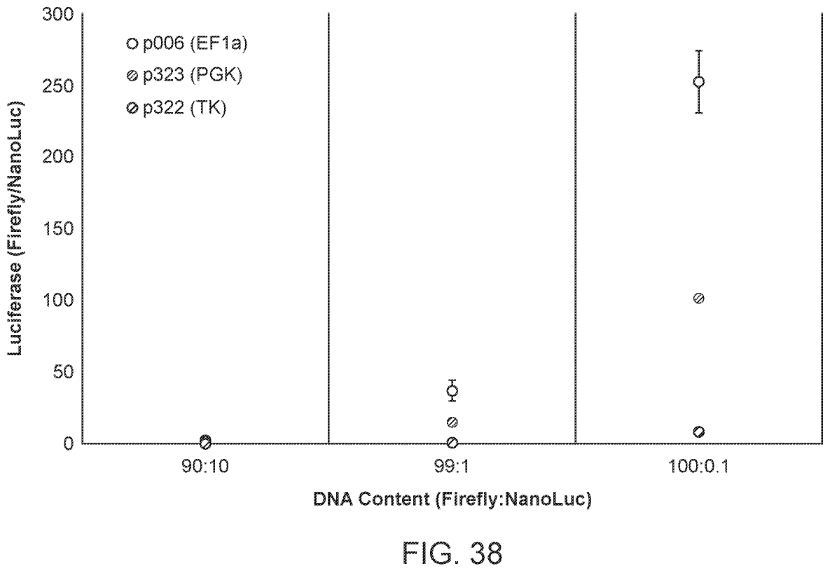

Promoter expression activity was assessed using a luciferase reporter assay. Characterization of the luciferase assay was performed by co-transfecting cells with a plasmid encoding Firefly luciferase and with a plasmid encoding NANOLUC® reporters. The luciferase reporters were under transcriptional control of standard promoters (EF1a, PGK, and TK). A standard curve of the normalized luciferase signal (Firefly signal/NANOLUC® signal) was generated using the following transfection ratios, 90 ng Firefly:10 ng NANOLUC®, 99 ng Firefly:1 ng NANOLUC®, and 100 ng Firefly:0.1 ng NANOLUC® (FIG. 38). Establishing such a ratiometric luciferase reporter assay allowed the determination of promoter expression activity without cross-signal interference.

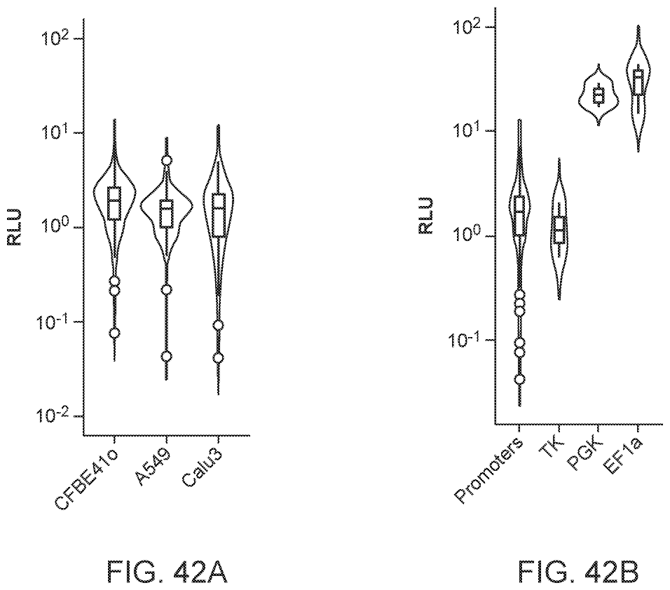

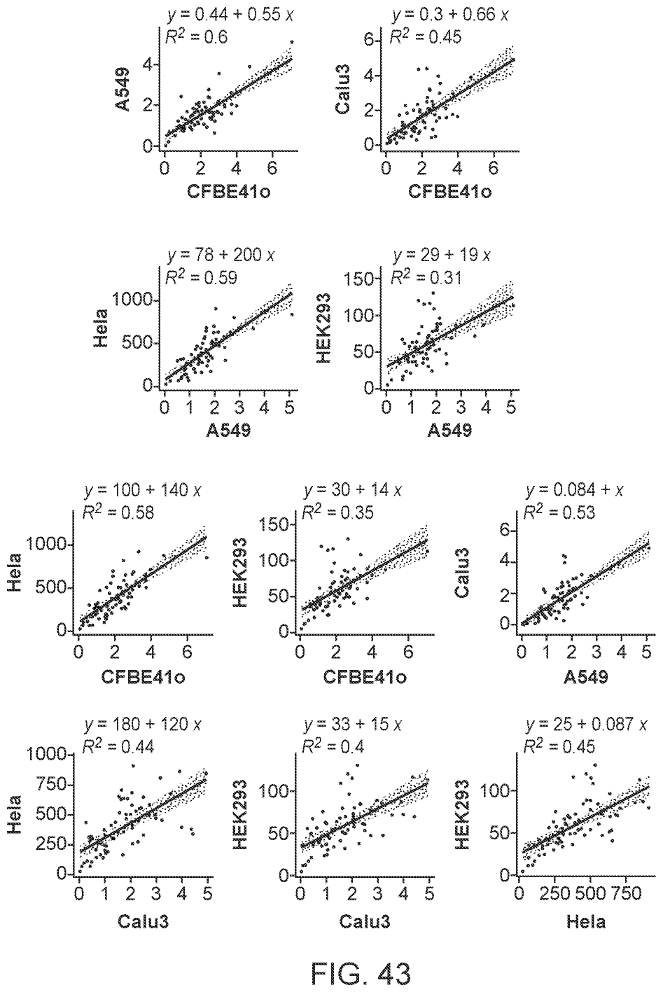

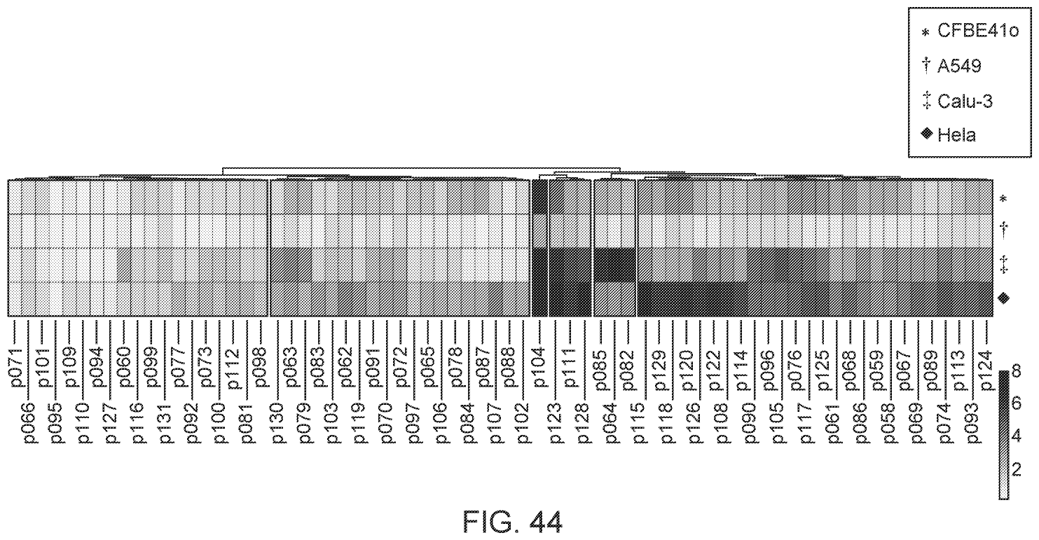

A library of 71 H1 promoters was then evaluated for expression activity in three lung cell types (A549, Calu-3, and CFBE4lo−) (FIGS. 39, 40, and 41) and two non-lung cell types (HEK293 and HeLa) used as control samples. Rank-order activity of the compact promoters in the library is shown in FIGS. 39, 40, and 41, along with activity of the standard TK promoter is shown (“TK”). Distributions of expression activity across the three lung cell types is shown in FIG. 42A. Of the 71 compact H1 promoters tested, 59 promoters in Calu-3 cells, 55 promoters in CFBE4lo− cells, and 11 in A549 cells exceeded TK controlled expression of luciferase reporter plasmids. The strongest promoters exceeded TK controlled expression activity by 2.5-8-fold and were only modestly weaker than the two strong standard promoters PGK and EF1a (FIG. 42B). The data suggests that most of the H1 promoters are active in lung cell lines. Furthermore, the promoters in this library do not contain viral or synthetic elements that can have negative consequences stemming from long-range enhancer activity. The data also showed that promoter activity was well-correlated among lung cell lines and across non-lung-cell types (FIG. 43). Hierarchical analysis (complete linkage clustering) was conducted to produce a heatmap as shown in FIG. 44. Through hierarchical analysis, a pattern suggesting that strong promoters in one cell type are likely to be strong promoters in other cell types emerged, enabling the clustering of promoters based on expression activity into six separate clusters (FIG. 44). Cluster 1 included promoters p071, p066, p101, p095, p109, p110, p094, p127, p060, p116, p099, p131, p077, p092, p073, p100, p112, p081, and p098. Cluster 2 included promoters p130, p063, p079, p083, p103, p062, p119, p091, p070, p072, p097, p065, p106, p078, p084, p087, p107, p088, and p102. Cluster 3 included promoter p104. Cluster 4 included promoters p123, p111, and p128. Cluster 5 included promoters p085, p064, and p082. Cluster 6 included promoters p115, p129, p118, p120, p126, p122, p108, p114, p090, p096, p105, p076, p117, p125, p061, p068, p086, p059, p058, p067, p069, p089, p074, p113, p093, and p124. Clusters 3-6 showed higher expression levels above the control TK p322 promoter.

Following clustering based on expression activity, the top five and bottom five promoters in A549 cells were identified, along with their respective ranking in four other cell types, as shown in TABLE 35.

TABLE 35
The top five and bottom five promoters in A549,
CFBE41o-, Calu-3, HeLa, and HEK293 cells.

| | | | | |
|---|---|---|---|---|
| A549 | CFBE41o- | Calu-3 | HeLa | HEK293 |

Top five promoters

| | | | | | |
|---|---|---|---|---|---|
| p104 | 1 | 1 | 1 | 3 | 5 |
| p123 | 2 | 2 | 5 | 2 | 10 |
| p111 | 3 | 10 | 6 | 7 | 20 |
| p128 | 4 | 24 | 8 | 4 | 11 |
| p118 | 5 | 6 | 31 | 10 | 23 |

Bottom five promoters

| | | | | | |
|---|---|---|---|---|---|
| p087 | 67 | 15 | 62 | 41 | 25 |
| p094 | 68 | 66 | 69 | 69 | 60 |
| p088 | 69 | 67 | 60 | 45 | 54 |
| p127 | 70 | 70 | 70 | 70 | 70 |
| p095 | 71 | 71 | 71 | 71 | 71 |

Wild type AAV genomes are ˜4.7 kb in length and recombinant AAV can package up to ˜5.2 kb. The DNA required to express full-length CFTR is comprised of the CFTR coding sequence (˜4.4 kb), two inverted terminal repeats (˜0.3 kb), a terminator (˜0.2 kb), and a promoter sequence. By adding the lengths of vector elements, it can be expected that the promoter lengths ≤232 bp may allow for full-length CFTR packaging; some elements, like the terminator sequence, can be further shortened. Given that AAV packaging efficiency may improve with smaller cassettes, a subset of promoters <200 bp was further analyzed and ranked as shown in TABLE 36.

TABLE 36
Ranked expression for ultra-compact (≤200 bp) promoters.
Ranked Expression

| | CFBE41o- | A549 | Calu-3 | HeLa | HEK293 | Size (bp) |
|---|---|---|---|---|---|---|
| p074 | 43 | 13 | 16 | 16 | 13 | 197 |
| p093 | 18 | 19 | 19 | 17 | 1 | 180 |
| p117 | 5 | 35 | 12 | 13 | 46 | 179 |
| p069 | 48 | 37 | 26 | 19 | 4 | 167 |
| p059 | 17 | 40 | 30 | 33 | 42 | 176 |

The compact promoters described herein are advantageous for their ability to drive expression of large proteins, such as CFTR, while allowing packaging in an AAV vector, circumventing long-standing challenges with AAV vector use for gene therapy applications. Many of the compact promoters described herein show expression levels at least as strong as a TK promoter (see, e.g., FIG. 42B). Further, a subset of the compact promoters described in the preceding example expressed in lung cells (FIG. 42A), a relevant cell type for CFTR gene therapy.

### Example 11. Generation of Ancestral H1 Promoter Sequences

This example describes the generation of synthetic H1 promoters (SEQ ID NOs: 936-1303) by reconstructing ancestral sequences from the H1 promoters herein described (e.g., SEQ ID NOs: 25-106, 469-476, 559-564, 609-614, 673-678, 681, 692-697, 706-711, 719-724, 729-734, 748-753, 784-789, 904-909, and 920-925).

First, a phylogenetic tree was built using RAxML or MEGA, as described in A. Stamatakis: “RAxML Version 8: A tool for Phylogenetic Analysis and Post-Analysis of Large Phylogenies” In Bioinformatics, 2014; Nei M. and Kumar S. (2000) Molecular Evolution and Phylogenetics Oxford University Press, New York; Tamura K., Stecher G., and Kumar S. (2021) MEGA 11: Molecular Evolutionary Genetics Analysis Version 11 Molecular Biology and Evolution (on the world wide web at doi.org/10.1093/molbev/msab120); and Stecher G., Tamura K., and Kumar S (2020) Molecular Evolutionary Genetics Analysis (MEGA) for macOS Molecular Biology and Evolution 37:1237-1239, herein incorporated by reference in their entireties.

For analysis with MEGA, the evolutionary history was inferred by using the Maximum Likelihood method and General Time Reversible model. The tree with the highest log likelihood (−25977.38) was selected. Initial tree(s) for the heuristic search were obtained automatically by applying Neighbor-Join and BioNJ algorithms to a matrix of pairwise distances estimated using the Maximum Composite Likelihood (MCL) approach, and then selecting the topology with superior log likelihood value. A discrete Gamma distribution was used to model evolutionary rate differences among sites (5 categories (+G, parameter=0.9471)). The rate variation model allowed for some sites to be evolutionarily invariable ([+1], 0.30% sites). This analysis involved 408 nucleotide sequences. There were a total of 467 positions in the final dataset. Evolutionary analyses were conducted in MEGA11.

The phyloFit program from PHAST (Phylogenetic Analysis with Space/Time Models) package was used to generate a phylogenetic model by fitting the tree models to the multiple sequence alignment by maximum likelihood using the HKY85 substitution model. The PREQUEL (Probabilistic REconstruction of ancestral seQUEnces, Largely) program from PHAST was used to compute marginal probability distributions for bases at ancestral nodes in the phylogenetic tree, using the tree model defined by phyloFit. Distributions were computed using the sum-product algorithm, assuming independence of sites. The identified sequences (SEQ ID NOs: 936-1303) correspond to nodes in the original tree.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

>CFTR_WT_CDNA_updated (SEQ ID NO: 1)
ATGCAGAGGTCGCCTCTGGAAAAGGCCAGCGTTGTCTCCAAACTTTTTTTCAGCTGGACCAGACCAATTTTGAGG
AAAGGATACAGACAGCGCCTGGAATTGTCAGACATATACCAAATCCCTTCTGTTGATTCTGCTGACAATCTATCT
GAAAAATTGGAAAGAGAATGGGATAGAGAGCTGGCTTCAAAGAAAAATCCTAAACTCATTAATGCCCTTCGGCGA
TGTTTTTTCTGGAGATTTATGTTCTATGGAATCTTTTTATATTTAGGGGAAGTCACCAAAGCAGTACAGCCTCTC
TTACTGGGAAGAATCATAGCTTCCTATGACCCGGATAACAAGGAGGAACGCTCTATCGCGATTTATCTAGGCATA
GGCTTATGCCTTCTCTTTATTGTGAGGACACTGCTCCTACACCCAGCCATTTTTGGCCTTCATCACATTGGAATG
CAGATGAGAATAGCTATGTTTAGTTTGATTTATAAGAAGACTTTAAAGCTGTCAAGCCGTGTTCTAGATAAAATA
AGTATTGGACAACTTGTTAGTCTCCTTTCCAACAACCTGAACAAATTTGATGAAGGACTTGCATTGGCACATTTC
GTGTGGATCGCTCCTTTGCAAGTGGCACTCCTCATGGGGCTAATCTGGGAGTTGTTACAGGCGTCTGCCTTCTGT
GGACTTGGTTTCCTGATAGTCCTTGCCCTTTTTCAGGCTGGGCTAGGGAGAATGATGATGAAGTACAGAGATCAG
AGAGCTGGGAAGATCAGTGAAAGACTTGTGATTACCTCAGAAATGATTGAAAATATCCAATCTGTTAAGGCATAC
TGCTGGGAAGAAGCAATGGAAAAAATGATTGAAAACTTAAGACAAACAGAACTGAAACTGACTCGGAAGGCAGCC
TATGTGAGATACTTCAATAGCTCAGCCTTCTTCTTCTCAGGGTTCTTTGTGGTGTTTTTATCTGTGCTTCCCTAT
GCACTAATCAAAGGAATCATCCTCCGGAAAATATTCACCACCATCTCATTCTGCATTGTTCTGCGCATGGCGGTC
ACTCGGCAATTTCCCTGGGCTGTACAAACATGGTATGACTCTCTTGGAGCAATAAACAAAATACAGGATTTCTTA
CAAAAGCAAGAATATAAGACATTGGAATATAACTTAACGACTACAGAAGTAGTGATGGAGAATGTAACAGCCTTC
TGGGAGGAGGGATTTGGGGAATTATTTGAGAAAGCAAAACAAAACAATAACAATAGAAAAACTTCTAATGGTGAT
GACAGCCTCTTCTTCAGTAATTTCTCACTTCTTGGTACTCCTGTCCTGAAAGATATTAATTTCAAGATAGAAAGA
GGACAGTTGTTGGCGGTTGCTGGATCCACTGGAGCAGGCAAGACTTCACTTCTAATGGTGATTATGGGAGAACTG
GAGCCTTCAGAGGGTAAAATTAAGCACAGTGGAAGAATTTCATTCTGTTCTCAGTTTTCCTGGATTATGCCTGGC
ACCATTAAAGAAAATATCATCTTTGGTGTTTCCTATGATGAATATAGATACAGAAGCGTCATCAAAGCATGCCAA
CTAGAAGAGGACATCTCCAAGTTTGCAGAGAAAGACAATATAGTTCTTGGAGAAGGTGGAATCACACTGAGTGGA
GGTCAACGAGCAAGAATTTCTTTAGCAAGAGCAGTATACAAAGATGCTGATTTGTATTTATTAGACTCTCCTTTT
GGATACCTAGATGTTTTAACAGAAAAAGAAATATTTGAAAGCTGTGTCTGTAAACTGATGGCTAACAAAACTAGG
ATTTTGGTCACTTCTAAAATGGAACATTTAAAGAAAGCTGACAAAATATTAATTTTGCATGAAGGTAGCAGCTAT
TTTTATGGGACATTTTCAGAACTCCAAAATCTACAGCCAGACTTTAGCTCAAAACTCATGGGATGTGATTCTTTC
GACCAATTTAGTGCAGAAAGAAGAAATTCAATCCTAACTGAGACCTTACACCGTTTCTCATTAGAAGGAGATGCT
CCTGTCTCCTGGACAGAAACAAAAAAACAATCTTTTAAACAGACTGGAGAGTTTGGGGAAAAAAGGAAGAATTCT
ATTCTCAATCCAATCAACTCTATACGAAAATTTTCCATTGTGCAAAAGACTCCCTTACAAATGAATGGCATCGAA
GAGGATTCTGATGAGCCTTTAGAGAGAAGGCTGTCCTTAGTACCAGATTCTGAGCAGGGAGAGGCGATACTGCCT
CGCATCAGCGTGATCAGCACTGGCCCCACGCTTCAGGCACGAAGGAGGCAGTCTGTCCTGAACCTGATGACACAC
TCAGTTAACCAAGGTCAGAACATTCACCGAAAGACAACAGCATCCACACGAAAAGTGTCACTGGCCCCTCAGGCA
AACTTGACTGAACTGGATATATATTCAAGAAGGTTATCTCAAGAAACTGGCTTGGAAATAAGTGAAGAAATTAAC
GAAGAAGACTTAAAGGAGTGCTTTTTTGATGATATGGAGAGCATACCAGCAGTGACTACATGGAACACATACCTT
CGATATATTACTGTCCACAAGAGCTTAATTTTTGTGCTAATTTGGTGCTTAGTAATTTTTCTGGCAGAGGTGGCT
GCTTCTTTGGTTGTGCTGTGGCTCCTTGGAAACACTCCTCTTCAAGACAAAGGGAATAGTACTCATAGTAGAAAT
AACAGCTATGCAGTGATTATCACCAGCACCAGTTCGTATTATGTGTTTTACATTTACGTGGGAGTAGCCGACACT
TTGCTTGCTATGGGATTCTTCAGAGGTCTACCACTGGTGCATACTCTAATCACAGTGTCGAAAATTTTACACCAC
AAAATGTTACATTCTGTTCTTCAAGCACCTATGTCAACCCTCAACACGTTGAAAGCAGGTGGGATTCTTAATAGA
TTCTCCAAAGATATAGCAATTTTGGATGACCTTCTGCCTCTTACCATATTTGACTTCATCCAGTTGTTATTAATT
GTGATTGGAGCTATAGCAGTTGTCGCAGTTTTACAACCCTACATCTTTGTTGCAACAGTGCCAGTGATAGTGGCT
TTTATTATGTTGAGAGCATATTTCCTCCAAACCTCACAGCAACTCAAACAACTGGAATCTGAAGGCAGGAGTCCA
ATTTTCACTCATCTTGTTACAAGCTTAAAAGGACTATGGACACTTCGTGCCTTCGGACGGCAGCCTTACTTTGAA
ACTCTGTTCCACAAAGCTCTGAATTTACATACTGCCAACTGGTTCTTGTACCTGTCAACACTGCGCTGGTTCCAA
ATGAGAATAGAAATGATTTTTGTCATCTTCTTCATTGCTGTTACCTTCATTTCCATTTTAACAACAGGAGAAGGA
GAAGGAAGAGTTGGTATTATCCTGACTTTAGCCATGAATATCATGAGTACATTGCAGTGGGCTGTAAACTCCAGC
ATAGATGTGGATAGCTTGATGCGATCTGTGAGCCGAGTCTTTAAGTTCATTGACATGCCAACAGAAGGTAAACCT
ACCAAGTCAACCAAACCATACAAGAATGGCCAACTCTCGAAAGTTATGATTATTGAGAATTCACACGTGAAGAAA
GATGACATCTGGCCCTCAGGGGGCCAAATGACTGTCAAAGATCTCACAGCAAAATACACAGAAGGTGGAAATGCC
ATATTAGAGAACATTTCCTTCTCAATAAGTCCTGGCCAGAGGGTGGGCCTCTTGGGAAGAACTGGATCAGGGAAG
AGTACTTTGTTATCAGCTTTTTTGAGACTACTGAACACTGAAGGAGAAATCCAGATCGATGGTGTGTCTTGGGAT
TCAATAACTTTGCAACAGTGGAGGAAAGCCTTTGGAGTGATACCACAGAAAGTATTTATTTTTTCTGGAACATTT
AGAAAAAACTTGGATCCCTATGAACAGTGGAGTGATCAAGAAATATGGAAAGTTGCAGATGAGGTTGGGCTCAGA
TCTGTGATAGAACAGTTTCCTGGGAAGCTTGACTTTGTCCTTGTGGATGGGGGCTGTGTCCTAAGCCATGGCCAC
AAGCAGTTGATGTGCTTGGCTAGATCTGTTCTCAGTAAGGCGAAGATCTTGCTGCTTGATGAACCCAGTGCTCAT
TTGGATCCAGTAACATACCAAATAATTAGAAGAACTCTAAAACAAGCATTTGCTGATTGCACAGTAATTCTCTGT
GAACACAGGATAGAAGCAATGCTGGAATGCCAACAATTTTTGGTCATAGAAGAGAACAAAGTGCGGCAGTACGAT
TCCATCCAGAAACTGCTGAACGAGAGGAGCCTCTTCCGGCAAGCCATCAGCCCCTCCGACAGGGTGAAGCTCTTT
CCCCACCGGAACTCAAGCAAGTGCAAGTCTAAGCCCCAGATTGCTGCTCTGAAAGAGGAGACAGAAGAAGAGGTG
CAAGATACAAGGCTTTAG
>CFTR_(deltaR) (SEQ ID NO: 2)
ATGCAGAGGTCGCCTCTGGAAAAGGCCAGCGTTGTCTCCAAACTTTTTTTCAGCTGGACCAGACCAATTTTGAGG
AAAGGATACAGACAGCGCCTGGAATTGTCAGACATATACCAAATCCCTTCTGTTGATTCTGCTGACAATCTATCT
GAAAAATTGGAAAGAGAATGGGATAGAGAGCTGGCTTCAAAGAAAAATCCTAAACTCATTAATGCCCTTCGGCGA
TGTTTTTTCTGGAGATTTATGTTCTATGGAATCTTTTTATATTTAGGGGAAGTCACCAAAGCAGTACAGCCTCTC
TTACTGGGAAGAATCATAGCTTCCTATGACCCGGATAACAAGGAGGAACGCTCTATCGCGATTTATCTAGGCATA
GGCTTATGCCTTCTCTTTATTGTGAGGACACTGCTCCTACACCCAGCCATTTTTGGCCTTCATCACATTGGAATG
CAGATGAGAATAGCTATGTTTAGTTTGATTTATAAGAAGACTTTAAAGCTGTCAAGCCGTGTTCTAGATAAAATA
AGTATTGGACAACTTGTTAGTCTCCTTTCCAACAACCTGAACAAATTTGATGAAGGACTTGCATTGGCACATTTC
GTGTGGATCGCTCCTTTGCAAGTGGCACTCCTCATGGGGCTAATCTGGGAGTTGTTACAGGCGTCTGCCTTCTGT
GGACTTGGTTTCCTGATAGTCCTTGCCCTTTTTCAGGCTGGGCTAGGGAGAATGATGATGAAGTACAGAGATCAG
AGAGCTGGGAAGATCAGTGAAAGACTTGTGATTACCTCAGAAATGATCGAGAACATCCAATCTGTTAAGGCATAC
TGCTGGGAAGAAGCAATGGAAAAAATGATTGAAAACTTAAGACAAACAGAACTGAAACTGACTCGGAAGGCAGCC
TATGTGAGATACTTCAATAGCTCAGCCTTCTTCTTCTCAGGGTTCTTTGTGGTGTTTTTATCTGTGCTTCCCTAT
GCACTAATCAAAGGAATCATCCTCCGGAAAATATTCACCACCATCTCATTCTGCATTGTTCTGCGCATGGCGGTC
ACTCGGCAATTTCCCTGGGCTGTACAAACATGGTATGACTCTCTTGGAGCAATAAACAAAATACAGGATTTCTTA
CAAAAGCAAGAATATAAGACATTGGAATATAACTTAACGACTACAGAAGTAGTGATGGAGAATGTAACAGCCTTC
TGGGAGGAGGGATTTGGGGAATTATTTGAGAAAGCAAAACAAAACAATAACAATAGAAAAACTTCTAATGGTGAT
GACAGCCTCTTCTTCAGTAATTTCTCACTTCTTGGTACTCCTGTCCTGAAAGATATTAATTTCAAGATAGAAAGA
GGACAGTTGTTGGCGGTTGCTGGATCCACTGGAGCAGGCAAGACTTCACTTCTAATGATGATTATGGGAGAACTG
GAGCCTTCAGAGGGTAAAATTAAGCACAGTGGAAGAATTTCATTCTGTTCTCAGTTTTCCTGGATTATGCCTGGC
ACCATTAAAGAAAATATCATCTTTGGTGTTTCCTATGATGAATATAGATACAGAAGCGTCATCAAAGCATGCCAA
CTAGAAGAGGACATCTCCAAGTTTGCAGAGAAAGACAATATAGTTCTTGGAGAAGGTGGAATCACACTGAGTGGA
GGTCAACGAGCAAGAATTTCTTTAGCAAGAGCAGTATACAAAGATGCTGATTTGTATTTATTAGACTCTCCTTTT
GGATACCTAGATGTTTTAACAGAAAAAGAAATATTTGAAAGCTGTGTCTGTAAACTGATGGCTAACAAAACTAGG
ATTTTGGTCACTTCTAAAATGGAACATTTAAAGAAAGCTGACAAAATATTAATTTTGCATGAAGGTAGCAGCTAT
TTTTATGGGACATTTTCAGAACTCCAAAATCTACAGCCAGACTTTAGCTCAAAACTCATGGGATGTGATTCTTTC
GACCAATTTAGTGCAGAAAGAAGAAATTCAATCCTAACTGAGACCTTACACCGTTTCTCATTAGAAGGAGATGCT
CCTGTCTCCTGGACAGAAACAAAAAAACAATCTTTTAAACAGACTGGAGAGTTTGGGGAAAAAAGGAAGAATTCT
ATTCTCAATCCAATCAACTCTACGCTTCAGGCACGAAGGAGGCAGTCTGTCCTGAACCTGATGACACACTCAGTT
AACCAAGGTCAGAACATTCACCGAAAGACAACAGCATCCACACGAAAAGTGTCACTGGCCCCTCAGGCAAACTTG
ACTGAACTGGATATATATTCAAGAAGGTTATCTCAAGAAACTGGCTTGGAAATAAGTGAAGAAATTAACGAAGAA
GACTTAAAGGAGTGCCTTTTTGATGATATGGAGAGCATACCAGCAGTGACTACATGGAACACATACCTTCGATAT
ATTACTGTCCACAAGAGCTTAATTTTTGTGCTAATTTGGTGCTTAGTAATTTTTCTGGCAGAGGTGGCTGCTTCT
TTGGTTGTGCTGTGGCTCCTTGGAAACACTCCTCTTCAAGACAAAGGGAATAGTACTCATAGTAGAAATAACAGC
TATGCAGTGATTATCACCAGCACCAGTTCGTATTATGTGTTTTACATTTACGTGGGAGTAGCCGACACTTTGCTT
GCTATGGGATTCTTCAGAGGTCTACCACTGGTGCATACTCTAATCACAGTGTCGAAAATTTTACACCACAAAATG
TTACATTCTGTTCTTCAAGCACCTATGTCAACCCTCAACACGTTGAAAGCAGGTGGGATTCTTAATAGATTCTCC
AAAGATATAGCAATTTTGGATGACCTTCTGCCTCTTACCATATTTGACTTCATCCAGTTGTTATTAATTGTGATT
GGAGCTATAGCAGTTGTCGCAGTTTTACAACCCTACATCTTTGTTGCAACAGTGCCAGTGATAGTGGCTTTTATT
ATGTTGAGAGCATATTTCCTCCAAACCTCACAGCAACTCAAACAACTGGAATCTGAAGGCAGGAGTCCAATTTTC
ACTCATCTTGTTACAAGCTTAAAAGGACTATGGACACTTCGTGCCTTCGGACGGCAGCCTTACTTTGAAACTCTG
TTCCACAAAGCTCTGAATTTACATACTGCCAACTGGTTCTTGTACCTGTCAACACTGCGCTGGTTCCAAATGAGA
ATAGAAATGATTTTTGTCATCTTCTTCATTGCTGTTACCTTCATTTCCATTTTAACAACAGGAGAAGGAGAAGGA
AGAGTTGGTATTATCCTGACTTTAGCCATGAATATCATGAGTACATTGCAGTGGGCTGTAAACTCCAGCATAGAT
GTGGATAGCTTGATGCGATCTGTGAGCCGAGTCTTTAAGTTCATTGACATGCCAACAGAAGGTAAACCTACCAAG
TCAACCAAACCATACAAGAATGGCCAACTCTCGAAAGTTATGATTATTGAGAATTCACACGTGAAGAAAGATGAC
ATCTGGCCCTCAGGGGGCCAAATGACTGTCAAAGATCTCACAGCAAAATACACAGAAGGTGGAAATGCCATATTA
GAGAACATTTCCTTCTCAATAAGTCCTGGCCAGAGGGTGGGCCTCTTGGGAAGAACTGGATCAGGGAAGAGTACT
TTGTTATCAGCTTTTTTGAGACTACTGAACACTGAAGGAGAAATCCAGATCGATGGTGTGTCTTGGGATTCAATA
ACTTTGCAACAGTGGAGGAAAGCCTTTGGAGTGATACCACAGAAAGTATTTATTTTTTCTGGAACATTTAGAAAA
AACTTGGATCCCTATGAACAGTGGAGTGATCAAGAAATATGGAAAGTTGCAGATGAGGTTGGGCTCAGATCTGTG
ATAGAACAGTTTCCTGGGAAGCTTGACTTTGTCCTTGTGGATGGGGGCTGTGTCCTAAGCCATGGCCACAAGCAG
TTGATGTGCTTGGCTAGATCTGTTCTCAGTAAGGCGAAGATCTTGCTGCTTGATGAACCCAGTGCTCATTTGGAT
CCAGTAACATACCAAATAATTAGAAGAACTCTAAAACAAGCATTTGCTGATTGCACAGTAATTCTCTGTGAACAC
AGGATAGAAGCAATGCTGGAATGCCAACAATTTTTGGTCATAGAAGAGAACAAAGTGCGGCAGTACGATTCCATC
CAGAAACTGCTGAACGAGAGGAGCCTCTTCCGGCAAGCCATCAGCCCCTCCGACAGGGTGAAGCTCTTTCCCCAC
CGGAACTCAAGCAAGTGCAAGTCTAAGCCCCAGATTGCTGCTCTGAAAGAGGAGACAGAAGAAGAGGTGCAAGAT
ACAAGGCTTTAG
>CFTR_recode-1 (SEQ ID NO: 3)
ATGCAGCGGTCACCCCTTGAGAAGGCATCGGTCGTGTCCAAGCTGTTCTTCAGCTGGACACGACCGATCCTTCGC
AAGGGGTACCGCCAGCGCTTGGAGCTGTCTGATATCTATCAGATTCCAAGCGTGGACAGCGCTGATAACCTCTCC
GAGAAGTTAGAGAGAGAGTGGGACCGTGAACTCGCCAGCAAGAAGAACCCAAAGCTGATCAATGCTTTGCGGCGC
TGCTTCTTCTGGCGGTTCATGTTCTACGGCATCTTTCTCTATCTCGGAGAGGTTACCAAGGCTGTCCAGCCGCTC
CTGCTGGGGCGGATAATTGCGAGCTACGATCCTGATGCGAAGGAGGAGCGCTCGATCGCCATTTATCTGGGCATC
GGGCTCTGCCTATTATTCATCGTGAGGACGTTGCTCCTGCACCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTCAGTCTTCTGTCAGCTGCCCTAGCTAAGTTTGACGAGGGCCTGGCTCTGGCACACTTC
GTGTGGATCGCGCCGCTGCAAGTGGCGCTGCTGATGGGCCTGATATGGGAGCTCTTGCAGGCGAGCGCGTTCTGT
GGCCTGGGGTTTCTCATCGTCCTGGCCTTGTTCCAGGCAGGCCTTGGACGGATGATGATGAAGTATCGCGATCAG
CGCGCTGGTAAGATCAGCGAGCGGCTGGTCATTACCAGCGAGATGATCGAGAACATTCAGTCCGTCAAGGCCTAC
TGCTGGGAGGAGGCCATGGAGAAGATGATAGAAGCCCTCAGGCAGACAGAGCTGAAGCTCACCCGCAAGGCTGCC
TATGTCAGGTACTTCAACAGCAGCGCCTTCTTTTTCAGCGGCTTCTTTGTGGTCTTCTTGTCAGTGCTACCGTAC
GCGTTGATAAAGGGGATCATCTTGAGGAAGATCTTCACGACGATCTCCTTTTGCATCGTGTTGCGGATGGCAGTG
ACAAGACAGTTTCCATGGGCCGTCCAGACTTGGTACGACAGTCTGGGCGCCATTGCGAAAATTCAGGATTTCCTG
CAGAAGCAGGAATACAAGACCCTCGAATACGCCCTCACGACCACGGAGGTGGTGATGGAGAACGTCACCGCCTTC
TGGGAGGAGGGCTTCGGGGAGCTGTTCGAGAAGGCCAAGCAGGCCAACGCGAACAGGAAAACGTCGAACGGGGAT
GATTCCCTGTTCTTCAGCAACTTCAGCTTGCTGGGCACGCCGGTCTTGAAGGACATCAACTTCAAGATCGAGCGT
GGCCAGCTGCTGGCAGTTGCTGGAAGCACGGGTGCAGGAAAGACGTCCTTACTGATGGTTATAATGGGCGAGCTC
GAGCCCAGTGAAGGCAAGATCAAGCACTCCGGTCGCATCAGTTTCTGCTCGCAATTTTCATGGATCATGCCAGGT
ACCATCAAGGAGAACATCATCTTTGGTGTCTCCTATGATGAGTACCGCTATCGCAGCGTCATCAAGGCTTGCCAG
CTGGAGGAGGACATCAGCAAGTTCGCGGAGAAGGATAACATCGTCCTGGGGGAAGGGGGAATTACCCTCAGTGGA
GGACAGAGGGCTCGTATCAGCCTTGCTCGGGCGGTCTATAAGGACGCTGACCTGTATCTGCTGGATTCGCCGTTC
GGCTACTTAGACGTGCTCACGGAGAAGGAGATCTTCGAGAGCTGCGTCTGTAAGCTGATGGCGGCGAAGACGCGG
ATACTGGTCACGTCCAAGATGGAGCACCTGAAGAAGGCTGATAAGATCCTCATCCTCCACGAGGGTAGTTCCTAC
TTCTACGGGACGTTCTCCGAGCTCCAGAACTTGCAGCCTGACTTCAGCAGCAAGCTGATGGGCTGCGATAGCTTT
GATCAGTTCTCCGCTGAGCGCCGAAACTCCATTCTGACTGAGACTCTGCACCGCTTCTCTCTGGAAGGAGACGCC
CCGGTCTCCTGGACAGAGACGAAGAAGCAGAGTTTCAAGCAGACTGGAGAGTTCGGCGAGAAGCGGAAGAACTCG
ATCCTGAATCCAATCGCCTCTATCCGGAAGTTCAGCATTGTCCAGAAGACGCCGTTGCAGATGAACGGCATCGAG
GAGGACAGTGATGAACCTCTGGAGAGGCGATTGTCCCTTGTTCCGGACTCGGAACAAGGGGAGGCGATCTTGCCG
CGGATTTCGGTGATTAGCACTGGCCCTACGCTACAGGCGAGGCGTCGTCAGAGCGTGCTGGCTCTGATGACGCAC
TCGGTAGCGCAGGGCCAGGCTATTCACCGAAAGACGACGGCAAGTACGCGTAAAGTGAGCCTGGCTCCTCAGGCG
GCGCTGACTGAGTTGGACATCTACAGTCGGCGCCTGTCTCAGGAGACCGGGCTCGAGATAAGTGAGGAGATAGCA
GAGGAGGACCTCAAGGAGTGCTTCTTTGATGATATGGAGAGTATCCCCGCGGTGACCACGTGGGCGACTTACCTC
CGTTACATCACGGTGCACAAGTCCTTGATCTTTGTCCTCATCTGGTGTCTCGTCATTTTTCTCGCCGAGGTAGCC
GCGAGCCTGGTGGTTCTGTGGCTCCTCGGCGCGACCCCGCTCCAGGACAAGGGGAACAGCACCCACTCGAGAAAC
GCATCGTATGCAGTGATTATCACGTCCACCTCCTCGTATTATGTATTCTACATATACGTGGGGGTGGCGGATACA
CTGCTTGCGATGGGTTTCTTTAGGGGGCTGCCCCTTGTCCATACGCTGATTACTGTCAGCAAGATTCTGCATCAC
AAGATGCTCCACAGTGTCCTGCAGGCGCCTATGTCAACGTTGGCTACGTTGAAGGCGGGCGGGATACTGAACAGG
TTCTCAAAGGACATAGCGATCTTGGATGATCTCTTGCCTTTGACCATTTTTGACTTTATCCAGCTGCTGCTGATC
GTGATAGGGGCCATCGCGGTCGTCGCGGTGCTGCAGCCATATATCTTTGTGGCTACAGTACCCGTGATCGTGGCC
TTTATCATGCTTCGGGCTTACTTCCTTCAGACTTCCCAGCAGCTGAAGCAGCTGGAGTCTGAAGGAAGAAGCCCG
ATTTTTACGCATCTGGTAACCAGCTTGAAGGGGCTGTGGACATTGCGGGCATTCGGGCGGCAGCCGTATTTTGAG
ACCTTGTTCCACAAGGCTCTCAATTTGCACACTGCCGCCTGGTTCCTGTATTTGTCCACGCTCCGCTGGTTCCAG
ATGCGTATCGAGATGATCTTCGTGATCTTCTTCATCGCCGTCACCTTCATCAGTATTCTGACGACTGGTGAGGGC
GAGGGGAGGGTCGGGATCATCTTGACGCTCGCGATGAACATCATGAGCACGCTCCAGTGGGCTGTCGCGTCGAGC
ATCGACGTCGACAGCCTCATGCGGAGCGTGTCTCGTGTGTTCAAGTTCATCGATATGCCGACCGAGGGAAAGCCG
ACCAAGAGTACGAAGCCGTACAAGAACGGTCAGCTTTCCAAGGTCATGATTATCGAGAACTCCCACGTGAAGAAG
GACGACATCTGGCCTTCTGGGGGACAGATGACCGTGAAGGACCTTACCGCCAAGTATACTGAGGGGGGTAATGCT
ATCCTGGAGGCCATATCGTTCTCGATCTCCCCCGGTCAGCGCGTGGGGCTGCTGGGGCGGACAGGGAGTGGTAAG
TCCACTCTCCTGTCCGCCTTCCTGCGGCTCCTCGCGACTGAGGGGGAGATCCAGATCGATGGGGTCTCCTGGGAT
AGCATTACCCTCCAGCAGTGGCGGAAGGCCTTCGGGGTCATCCCCCAGAAGGTCTTCATCTTCAGTGGGACGTTT
CGTAAGGCGTTGGACCCTTACGAACAGTGGTCGGATCAAGAGATATGGAAGGTCGCCGACGAGGTGGGTCTTCGC
TCCGTTATTGAGCAGTTTCCGGGCAAGCTAGACTTCGTCTTAGTTGACGGTGGCTGTGTGTTGTCACACGGCCAC
AAGCAGCTGATGTGTCTAGCTCGCTCGGTGCTCAGTAAAGCGAAGATCCTCTTGTTGGACGAGCCTTCCGCCCAT
CTTGATCCGGTCACTTATCAGATCATCAGGCGGACACTCAAGCAGGCCTTTGCGGATTGCACAGTGATTCTGTGC
GAGCACCGCATAGAGGCCATGCTTGAGTGTCAGCAGTTTCTGGTGATCGAGGAGGCTAAGGTCCGCCAGTATGAT
TCGATCCAGAAGTTACTGGCGGAGCGTAGCCTCTTCCGACAGGCGATCTCGCCGTCGGATAGAGTCAAGCTGTTT
CCTCACCGGGCCTCCTCGAAGTGTAAGTCAAAGCCGCAGATTGCGGCTTTGAAGGAGGAGACTGAGGAGGAGGTC
CAGGACACGCGGCTCTAA
>CFTR_recode-2 (SEQ ID NO: 4)
ATGCAGCGGTCACCCCTTGAGAAGGCATCGGTCGTGTCCAAGCTGTTCTTCAGCTGGACACGACCGATCCTTCGC
AAGGGGTACCGCCAGCGCTTGGAGCTGTCTGATATCTATCAGATTCCAAGCGTGGACAGCGCTGATAACCTCTCC
GAGAAGTTAGAGAGAGAGTGGGACCGTGAACTCGCCAGCAAGAAGAACCCAAAGCTGATCAATGCTTTGCGGCGC
TGCTTCTTCTGGCGGTTCATGTTCTACGGCATCTTTCTCTATCTCGGAGAGGTTACCAAGGCTGTCCAGCCGCTC
CTGCTGGGGCGGATAATTGCGAGCTACGATCCTGATGCGAAGGAGGAGCGCTCGATCGCCATTTATCTGGGCATC
GGGCTCTGCCTATTATTCATCGTGAGGACGTTGCTCCTGCACCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTCAGTCTTCTGTCAGCTGCCCTAGCTAAGTTTGACGAGGGCCTGGCTCTGGCACACTTC
GTGTGGATCGCGCCGCTGCAAGTGGCGCTGCTGATGGGCCTGATATGGGAGCTCTTGCAGGCGAGCGCGTTCTGT
GGCCTGGGGTTTCTCATCGTCCTGGCCTTGTTCCAGGCAGGCCTTGGACGGATGATGATGAAGTATCGCGATCAG
CGCGCTGGTAAGATCAGCGAGCGGCTGGTCATTACCAGCGAGATGATCGAGAACATTCAGTCCGTCAAGGCCTAC
TGCTGGGAGGAGGCCATGGAGAAGATGATAGAAGCCCTCAGGCAGACAGAGCTGAAGCTCACCCGCAAGGCTGCC
TATGTCAGGTACTTCAACAGCAGCGCCTTCTTTTTCAGCGGCTTCTTTGTGGTCTTCTTGTCAGTGCTACCGTAC
GCGTTGATAAAGGGGATCATCTTGAGGAAGATCTTCACGACGATCTCCTTTTGCATCGTGTTGCGGATGGCAGTG
ACAAGACAGTTTCCATGGGCCGTCCAGACTTGGTACGACAGTCTGGGCGCCATTGCGAAAATTCAGGATTTCCTG
CAGAAGCAGGAATACAAGACCCTCGAATACGCCCTCACGACCACGGAGGTGGTGATGGAGAACGTCACCGCCTTC
TGGGAGGAGGGCTTCGGGGAGCTGTTCGAGAAGGCCAAGCAGGCCAACGCGAACAGGAAAACGTCGAACGGGGAT
GATTCCCTGTTCTTCAGCAACTTCAGCTTGCTGGGCACGCCGGTCTTGAAGGACATCAACTTCAAGATCGAGCGT
GGCCAGCTGCTGGCAGTTGCTGGAAGCACGGGTGCAGGAAAGACGTCCTTACTGATGGTTATAATGGGCGAGCTC
GAGCCCAGTGAAGGCAAGATCAAGCACTCCGGTCGCATCAGTTTCTGCTCGCAATTCTCATGGATCATGCCCGGT
ACCATCAAGGAGAACATCATCTTTGGTGTCTCCTATGATGAGTACCGGTATCGATCCGTGATTAAGGCTTGTCAG
CTGGAGGAGGATATCTCCAAGTTCGCCGAGAAGGATAACATCGTTCTCGGCGAAGGAGGTATCACCCTCTCTGGT
GGCCAGAGAGCGCGTATTTCGCTGGCCAGAGCTGTATACAAGGACGCGGATCTCTACCTTCTCGACAGCCCGTTC
GGTTATCTGGACGTGCTGACCGAGAAGGAGATATTCGAGTCCTGTGTGTGCAAGCTCATGGCCGCGAAGACGCGC
ATTCTGGTCACCAGTAAAATGGAGCATCTGAAGAAGGCCGATAAGATACTGATTCTCCACGAGGGGAGTTCGTAT
TTTTACGGCACTTTTTCAGAGCTCCAAAATCTTCAGCCCGATTTCTCCAGTAAGCTTATGGGCTGCGATTCGTTT
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGTCCTTTAAGCAGACTGGAGAATTCGGGGAGAAGAGGAAAAATTCG
ATACTGAACCCCATTGCGAGCATTCGCAAGTTCAGTATCGTGCAGAAGACGCCTCTCCAGATGAACGGAATCGAG
GAGGACTCAGACGAGCCGCTGGAGCGGCGGCTCAGTCTGGTCCCCGATTCCGAGCAGGGAGAGGCGATTCTGCCA
CGCATCAGCGTGATCTCCACCGGGCCTACCCTGCAGGCCCGGCGGAGACAGAGTGTCCTAGCTCTGATGACGCAC
TCTGTGGCGCAAGGGCAGGCCATCCATCGTAAGACGACCGCGTCTACGAGGAAGGTCTCCCTTGCGCCACAGGCT
GCGTTAACAGAGCTGGACATTTATTCGAGGAGGCTCTCCCAGGAGACCGGCTTGGAGATCTCGGAGGAGATCGCC
GAGGAGGATCTCAAGGAGTGCTTCTTCGACGATATGGAGAGCATACCAGCAGTGACGACGTGGGCGACTTATTTA
CGGTATATTACCGTACATAAGTCGCTCATTTTCGTCCTGATCTGGTGTCTCGTCATATTTCTGGCGGAGGTCGCG
GCGTCGCTGGTTGTGTTGTGGCTGTTGGGTGCTACTCCCCTGCAGGACAAGGGGAATAGCACCCACAGCCGCAAC
GCCAGCTACGCCGTGATCATCACCAGCACAAGCTCTTACTATGTCTTCTACATCTATGTCGGGGTGGCGGACACG
CTGCTGGCCATGGGTTTCTTCCGTGGCCTGCCGCTTGTCCACACCCTGATAACGGTGTCGAAGATATTGCATCAC
AAGATGCTCCATTCAGTGTTGCAAGCGCCGATGTCGACGCTTGCAACACTGAAGGCGGGGGGGATCCTCAACCGG
TTTAGCAAGGACATTGCTATACTGGATGATCTCCTCCCGCTGACTATCTTTGACTTCATCCAGCTGCTGCTGATC
GTGATCGGGGCGATAGCCGTGGTCGCGGTCTTGCAGCCGTACATCTTCGTGGCCACAGTCCCTGTGATAGTGGCG
TTCATCATGCTGAGGGCGTACTTCCTTCAGACTTCCCAGCAGCTGAAGCAGCTGGAGTCTGAAGGAAGGTCGCCC
ATCTTCACGCATTTGGTGACGTCACTCAAGGGACTGTGGACCTTGAGGGCGTTCGGCAGGCAGCCGTATTTCGAG
ACCTTGTTCCACAAGGCCTTGAACCTGCATACGGCTGCCTGGTTCCTCTACCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGGAGGGTCGGGATCATCCTGACCCTCGCTATGAACATCATGAGCACGCTCCAGTGGGCTGTCGCGTCGAGC
ATCGACGTCGACAGCCTCATGCGGAGCGTGTCTCGTGTGTTCAAGTTCATAGATATGCCGACGGAGGGTAAGCCC
ACTAAAAGTACGAAGCCCTACAAGAACGGCCAGCTGTCCAAGGTGATGATTATCGAGAACAGTCACGTCAAGAAG
GACGACATCTGGCCCTCGGGTGGGCAGATGACGGTGAAGGACCTCACTGCCAAGTACACCGAGGGCGGGAACGCC
ATTCTTGAGGCGATCTCGTTCTCGATATCACCTGGACAGCGGGTCGGCTTGTTGGGCCGTACTGGTAGTGGGAAG
TCGACCCTCCTGTCGGCATTTCTTCGCCTTCTGGCCACAGAAGGCGAAATCCAGATCGATGGGGTGTCCTGGGAT
TCCATAACGTTGCAGCAGTGGAGGAAGGCCTTCGGGGTGATTCCCCAGAAGGTCTTCATCTTCTCTGGGACGTTT
CGGAAGGCCCTGGACCCCTACGAGCAGTGGAGCGACCAGGAGATCTGGAAGGTCGCGGACGAGGTGGGCCTGCGG
AGCGTCATAGAGCAGTTCCCTGGAAAGCTCGACTTCGTGCTGGTAGACGGCGGTTGTGTGCTGTCCCACGGCCAC
AAGCAGCTGATGTGCCTAGCACGAAGCGTGCTTTCCAAGGCGAAGATACTGCTCTTGGACGAGCCGTCGGCCCAC
CTCGACCCCGTGACCTACCAGATCATCCGTCGCACACTCAAGCAGGCCTTTGCGGATTGCACAGTGATTCTGTGC
GAGCACCGCATAGAGGCCATGCTTGAGTGTCAGCAGTTCCTCGTGATCGAGGAAGCTAAAGTCCGCCAGTACGAC
TCGATCCAGAAGTTGCTGGCGGAGCGGTCCCTCTTTCGGCAGGCGATCTCGCCTTCGGACCGCGTTAAGCTGTTC
CCCCACCGGGCGAGCAGCAAATGCAAGTCCAAGCCGCAGATCGCCGCGCTGAAAGAGGAGACCGAGGAGGAAGTC
CAAGATACTCGGCTCTAA
>CFTR_recode-3 (SEQ ID NO: 5)
ATGCAGAGGTCGCCCCTTGAGAAGGCATCGGTCGTGTCCAAGCTGTTCTTCAGCTGGACACGACCGATCCTTCGC
AAGGGGTACCGCCAGCGACTGGAGTTGTCTGATATCTATCAGATACCTTCAGTCGACTCGGCGGATAACCTCTCC
GAGAAGTTAGAGAGAGAGTGGGACCGTGAACTCGCCAGCAAGAAGAACCCAAAGCTGATCAATGCTTTGCGGCGC
TGCTTCTTCTGGCGGTTCATGTTCTACGGCATCTTTCTCTATCTCGGAGAGGTTACGAAGGCGGTGCAGCCGCTC
CTGCTGGGGCGGATCATCGCCTCGTATGACCCAGACGCGAAGGAGGAGCGCTCCATCGCGATCTATCTGGGCATA
GGCCTCTGCCTCCTCTTTATTGTTCGTACCTTGCTTCTGCACCCCGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTCAGTCTTCTGTCAGCTGCCCTAGCTAAGTTTGACGAGGGCCTGGCTCTGGCACACTTC
GTGTGGATCGCTCCACTCCAGGTGGCGCTGTTGATGGGCCTGATATGGGAGCTCTTGCAGGCGAGCGCGTTCTGT
GGCCTGGGGTTTCTCATCGTCCTGGCCTTGTTCCAGGCAGGCCTTGGACGGATGATGATGAAGTATCGCGATCAG
CGCGCTGGTAAGATCAGCGAGCGGCTGGTCATTACCAGCGAGATGATCGAGAACATTCAGTCCGTCAAGGCCTAC
TGCTGGGAGGAGGCCATGGAGAAGATGATAGAAGCCCTCAGGCAGACAGAGCTGAAGCTCACCCGCAAGGCTGCC
TATGTCAGGTACTTCAACAGCTCCGCCTTCTTCTTCTCAGGGTTCTTTGTCGTGTTTCTGTCCGTCCTCCCCTAC
GCGTTGATAAAGGGGATCATCTTGAGGAAGATCTTCACGACGATCTCCTTTTGCATCGTGTTGAGGATGGCAGTG
ACACGACAATTCCCCTGGGCCGTGCAGACATGGTACGACAGTTTAGGGGCTATTGCGAAGATCCAGGATTTTCTG
CAGAAGCAAGAGTACAAGACGCTGGAATACGCCCTCACGACCACGGAGGTGGTCATGGAGAACGTGACCGCCTTC
TGGGAGGAGGGCTTTGGTGAGCTCTTCGAGAAGGCCAAGCAGGCCAACGCCAATCGGAAGACTAGTAACGGCGAC
GACTCCCTGTTCTTCAGCAACTTCAGCTTGCTGGGCACGCCGGTCTTGAAGGACATCAACTTCAAGATCGAGCGT
GGCCAGCTGCTGGCAGTTGCTGGGAGCACGGGAGCGGGCAAGACTAGTCTTCTGATGGTGATCATGGGCGAGCTT
GAGCCTTCCGAAGGGAAGATCAAACATTCAGGCCGGATCAGCTTCTGTAGCCAGTTCTCGTGGATCATGCCGGGG
ACGATAAAGGAGAATATCATCTTCGGCGTGAGCTACGACGAATACCGCTACAGAAGCGTGATCAAGGCCTGCCAG
CTCGAGGAGGATATCTCCAAGTTCGCCGAGAAGGATAACATCGTTCTCGGCGAAGGAGGTATCACCTTGAGCGGC
GGTCAGCGCGCCCGGATAAGCCTGGCACGGGCTGTCTATAAAGATGCAGACTTGTATCTTTTAGACAGCCCGTTC
GGGTATCTGGACGTGCTGACCGAAAAGGAGATCTTCGAGTCGTGCGTTTGTAAGCTGATGGCGGCCAAGACGAGG
ATTCTTGTCACCTCTAAGATGGAGCATCTTAAGAAGGCTGACAAGATCCTCATTCTGCACGAGGGGAGCTCCTAC
TTCTATGGGACGTTCAGCGAGTTGCAGAATCTTCAGCCCGATTTCTCCAGTAAGCTTATGGGCTGTGACTCCTTC
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGAGCTTTAAGCAGACTGGAGAATTCGGGGAGAAGAGAAAGAACTCG
ATCCTGAACCCCATAGCAAGTATCAGGAAGTTCTCCATCGTGCAGAAAACGCCCCTGCAGATGAACGGGATCGAG
GAGGACAGTGATGAGCCGCTAGAACGGAGGCTGTCGCTGGTGCCTGACTCAGAGCAGGGCGAGGCGATATTACCG
AGAATCTCGGTGATATCGACTGGCCCTACTCTGCAGGCCCGGCGACGGCAGTCCGTTCTAGCGCTCATGACCCAC
TCGGTCGCGCAGGGGCAGGCCATCCATCGGAAGACAACCGCATCGACTCGAAAGGTCTCCTTAGCGCCGCAGGCG
GCGCTAACGGAGCTCGACATATATTCGAGGAGGCTCTCCCAGGAGACCGGCTTGGAGATCTCGGAGGAGATCGCC
GAGGAGGATCTCAAGGAGTGCTTCTTCGATGATATGGAGAGCATTCCAGCGGTGACGACTTGGGCTACTTACCTG
CGCTACATCACGGTGCACAAGTCGCTGATCTTCGTGCTGATCTGGTGCTTGGTAATTTTCCTTGCCGAAGTGGCG
GCGTCCCTAGTAGTCTTGTGGCTACTAGGGGCGACGCCACTTCAGGACAAGGGAAATTCCACGCACTCACGTAAT
GCGAGCTATGCGGTGATCATCACCAGCACTAGCTCGTATTACGTGTTCTACATCTATGTCGGGGTGGCGGACACG
CTGCTGGCCATGGGATTCTTCCGTGGCCTGCCGCTTGTCCACACCCTGATAACGGTGTCGAAGATACTGCATCAC
AAGATGCTCCACAGCGTGCTCCAAGCACCGATGAGCACGCTGGCGACGTTGAAGGCGGGGGGGATCCTCAACAGG
TTTTCCAAGGATATCGCTATCCTGGATGACCTGTTGCCCCTCACGATCTTCGACTTCATCCAGCTGTTGCTCATC
GTGATTGGAGCAATCGCTGTGGTAGCAGTGTTGCAGCCCTATATCTTCGTGGCGACTGTGCCCGTGATAGTAGCG
TTTATCATGCTGCGGGCCTACTTCTTGCAGACTTCACAGCAGTTGAAGCAGCTGGAGTCGGAGGGGCGGTCGCCC
ATATTCACGCACCTGGTCACGAGCCTGAAGGGGCTCTGGACCTTGCGTGCGTTTGGGCGACAGCCCTACTTCGAG
ACCCTGTTTCATAAGGCTTTGAATCTGCATACCGCAGCATGGTTCCTGTACCTCTCCACTCTCCGGTGGTTTCAG
ATGCGGATTGAGATGATCTTCGTGATTTTCTTTATCGCGGTGACATTCATTTCGATTCTGACCACCGGAGAGGGA
GAGGGCAGGGTGGGCATAATCCTCACTCTGGCTATGAACATCATGTCGACGCTACAGTGGGCTGTAGCGTCGAGC
ATAGATGTTGATAGCCTGATGAGGAGTGTGTCCAGGGTATTCAAGTTCATTGACATGCCCACGGAGGGTAAGCCT
ACGAAGAGCACCAAGCCGTACAAGAACGGCCAGCTCTCGAAGGTAATGATCATCGAGAACAGCCATGTGAAGAAG
GACGACATATGGCCCTCAGGAGGGCAGATGACAGTGAAGGACCTCACTGCCAAGTACACCGAGGGGGGCAACGCG
ATCCTCGAGGCGATATCGTTCTCGATATCGCCCGGGCAGCGCGTTGGCCTCCTCGGTCGTACTGGCAGTGGGAAG
TCCACACTGTTATCTGCCTTCCTGAGGCTTCTCGCCACTGAGGGAGAGATCCAGATTGATGGAGTCTCCTGGGAC
TCCATCACTCTCCAGCAGTGGCGGAAGGCCTTCGGGGTGATTCCCCAGAAGGTGTTCATATTTTCAGGGACCTTT
CGTAAGGCCCTGGACCCGTATGAGCAGTGGTCGGATCAGGAGATCTGGAAGGTAGCAGACGAGGTCGGCCTTCGG
AGCGTGATCGAGCAGTTCCCAGGTAAGCTGGACTTTGTGCTCGTGGATGGGGGCTGCGTCCTCAGCCACGGGCAC
AAACAGCTTATGTGTCTTGCTCGATCAGTGCTCTCGAAGGCCAAGATCTTGCTGCTAGACGAGCCTTCCGCGCAT
CTCGATCCGGTCACTTACCAAATCATTCGGCGAACGCTGAAGCAAGCGTTCGCCGATTGCACCGTGATCCTGTGT
GAGCACAGGATCGAGGCGATGCTGGAGTGCCAGCAGTTCTTGGTGATCGAGGAGGCCAAGGTGCGGCAGTACGAC
TCGATCCAGAAGCTTCTGGCGGAGAGAAGCCTGTTCCGTCAGGCTATCTCTCCGTCAGATCGGGTCAAGTTGTTT
CCGCACCGGGCCTCCTCGAAATGCAAGTCAAAGCCGCAGATAGCGGCTTTGAAAGAAGAAACAGAGGAGGAGGTC
CAGGACACCCGCCTCTGA
>CFTR_recode-4 (SEQ ID NO: 6)
ATGCAGAGGTCGCCCCTTGAGAAGGCATCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATTCAGGATTTCCTG
CAGAAGCAGGAATACAAGACCCTCGAATACGCCCTCACGACCACGGAGGTGGTGATGGAGAACGTCACCGCCTTC
TGGGAGGAGGGCTTCGGGGAGCTGTTCGAGAAGGCCAAGCAGGCCAACGCGAACCGCAAGACCTCGAACGGGGAT
GATTCCCTGTTCTTCAGCAACTTCAGCCTGCTGGGCACGCCGGTCTTGAAGGACATCAACTTCAAGATCGAGCGT
GGCCAGCTGCTGGCAGTTGCTGGAAGTACGGGCGCTGGCAAGACTTCCCTTCTGATGGTGATAATGGGCGAATTA
GAGCCATCAGAAGGGAAGATCAAGCACAGCGGCCGTATTAGCTTCTGCAGCCAGTTCAGCTGGATTATGCCAGGC
ACCATCAAGGAGAATATCATATTCGGTGTATCGTACGACGAGTATCGGTATCGGTCGGTCATCAAGGCATGCCAG
CTCGAGGAGGATATCTCCAAGTTCGCCGAGAAGGACAACATCGTGTTAGGCGAAGGGGGAATCACGCTAAGTGGA
GGTCAGCGTGCTCGTATATCGCTCGCCAGAGCGGTATACAAGGACGCTGACCTCTACTTGCTTGATTCCCCCTTC
GGCTATCTCGATGTCCTTACGGAGAAGGAGATATTCGAGAGCTGTGTATGCAAGTTGATGGCCGCCAAGACGCGG
ATACTCGTCACGAGTAAGATGGAGCACCTCAAGAAGGCAGATAAGATTCTGATCCTTCATGAGGGCTCCTCTTAC
TTCTATGGGACGTTCAGTGAGCTGCAGAATCTTCAGCCCGATTTCTCCAGTAAGCTTATGGGCTGTGACTCCTTC
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGAGCTTTAAGCAGACTGGAGAATTCGGGGAGAAGAGAAAGAACAGC
ATACTGAACCCCATAGCTTCCATCCGGAAGTTCAGCATCGTCCAGAAGACTCCTCTCCAGATGAATGGAATCGAG
GAGGATTCGGACGAGCCCCTGGAGCGTCGGCTCAGTCTGGTCCCGGATTCCGAGCAGGGGGAGGCCATCCTCCCT
CGGATTTCGGTGATCAGCACTGGGCCGACGCTCCAGGCCCGGCGCCGTCAGAGCGTCCTGGCGCTGATGACACAC
AGCGTGGCGCAGGGTCAGGCGATCCATCGCAAGACCACTGCGTCCACGCGGAAGGTGTCATTAGCGCCACAGGCC
GCTCTGACGGAGCTGGATATCTACAGCCGCCGTCTCTCCCAGGAGACGGGGCTGGAGATATCGGAGGAGATCGCC
GAGGAGGACCTCAAGGAGTGCTTCTTCGACGACATGGAGAGCATTCCTGCGGTCACTACCTGGGCGACCTACCTC
CGATACATCACGGTACACAAGAGTTTGATATTCGTCTTGATATGGTGCCTGGTCATATTCCTAGCTGAAGTGGCT
GCATCGCTCGTGGTGTTGTGGCTGTTGGGTGCTACTCCCCTGCAGGACAAGGGGAATAGCACCCACAGCCGCAAC
GCCTCGTACGCGGTGATCATCACCAGTACGAGCTCCTACTATGTGTTCTACATATATGTAGGAGTTGCTGACACG
TTGTTAGCGATGGGCTTCTTCAGGGGGTTACCCCTGGTCCATACGCTGATAACGGTCAGCAAGATCCTGCACCAC
AAGATGCTCCATTCAGTGTTGCAAGCGCCGATGTCGACGCTTGCAACACTGAAGGCTGGAGGCATCTTGAACAGG
TTCTCGAAGGACATTGCAATACTGGACGACCTATTGCCCCTGACGATCTTCGACTTCATCCAGTTGTTGTTGATC
GTCATAGGGGCAATAGCGGTCGTCGCAGTATTGCAGCCGTACATCTTTGTCGCCACTGTGCCAGTTATCGTCGCC
TTCATAATGTTGAGAGCCTACTTCCTTCAGACTAGTCAGCAGCTGAAGCAGTTGGAGAGCGAGGGTCGCTCTCCA
ATCTTCACGCACTTGGTGACTAGTCTGAAGGGGCTCTGGACATTGCGGGCATTCGGGCGGCAGCCGTATTTTGAG
ACCTTGTTCCACAAGGCTCTCAATTTACACACTGCCGCCTGGTTCCTGTACCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGGCGAGTGGGGATCATTCTCACACTCGCCATGAACATCATGAGCACGCTCCAGTGGGCTGTCGCGTCGAGC
ATCGACGTCGACAGCCTCATGCGGAGCGTGTCTCGTGTGTTCAAGTTCATCGATATGCCTACGGAGGGCAAGCCC
ACCAAGAGTACGAAGCCGTACAAGAATGGGCAGCTCTCCAAGGTAATGATTATCGAGAACTCCCATGTGAAGAAG
GACGATATCTGGCCCAGTGGCGGCCAGATGACGGTTAAAGACCTTACTGCGAAGTACACCGAAGGCGGCAACGCC
ATTCTGGAGGCGATATCGTTTTCCATCTCACCCGGTCAGCGGGTCGGCCTCCTCGGTCGCACGGGGTCGGGGAAG
AGCACTCTGCTGAGTGCTTTTCTCCGACTCCTTGCGACCGAGGGGGAGATCCAGATTGACGGGGTGAGTTGGGAT
TCGATAACCCTCCAGCAGTGGCGTAAGGCCTTCGGTGTAATTCCGCAGAAGGTCTTTATCTTCTCGGGGACCTTC
CGGAAGGCTCTTGATCCGTACGAGCAATGGTCGGATCAAGAGATCTGGAAGGTCGCCGACGAAGTCGGGCTTCGC
TCGGTGATCGAGCAGTTCCCGGGCAAGCTCGACTTCGTCTTGGTCGATGGAGGGTGCGTTCTGAGCCACGGCCAT
AAGCAGCTTATGTGCCTGGCTCGGAGCGTACTCTCCAAGGCCAAGATTCTGCTCTTGGATGAGCCGAGTGCTCAC
CTGGATCCGGTGACCTACCAGATCATTCGGCGAACGCTGAAGCAAGCGTTCGCCGATTGCACCGTGATCCTGTGT
GAGCACAGGATCGAGGCCATGCTGGAGTGCCAGCAGTTCCTCGTGATCGAGGAAGCGAAGGTACGCCAGTATGAC
TCGATCCAGAAGCTGCTGGCGGAGAGATCCCTGTTCCGTCAGGCGATCTCTCCGTCAGACCGGGTGAAGCTATTT
CCTCATCGGGCCTCCTCGAAGTGCAAGTCAAAGCCGCAGATTGCGGCTTTGAAGGAGGAGACCGAGGAGGAAGTG
CAGGACACCCGGTTATAA
>CFTR_recode-5 (SEQ ID NO: 7)
ATGCAGAGGTCGCCCCTTGAGAAGGCATCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATTCAGGATTTCCTG
CAGAAGCAGGAATACAAGACCCTCGAATACGCCCTCACGACCACGGAGGTGGTGATGGAGAACGTCACCGCCTTC
TGGGAGGAGGGCTTCGGGGAGCTGTTCGAGAAGGCCAAGCAGGCCAACGCGAACCGCAAGACCTCGAACGGGGAT
GATTCCCTGTTCTTCAGCAACTTCAGCCTGCTGGGCACGCCGGTCTTGAAGGACATCAACTTCAAGATCGAGCGT
GGCCAGCTGCTGGCAGTTGCTGGAAGTACGGGCGCTGGCAAGACTTCCCTTCTGATGGTGATAATGGGCGAATTA
GAGCCATCAGAAGGGAAGATCAAGCACAGCGGCCGTATTAGCTTCTGCAGCCAGTTCAGCTGGATTATGCCAGGC
ACCATCAAGGAGAATATCATATTCGGTGTATCGTACGACGAGTATCGGTATCGGTCGGTCATCAAGGCATGCCAG
CTCGAGGAGGATATCTCCAAGTTCGCCGAGAAGGACAACATCGTGTTAGGCGAAGGGGGAATCACGCTAAGTGGA
GGTCAGCGTGCTCGTATATCGCTCGCCAGAGCGGTATACAAGGACGCTGACCTCTACTTGCTTGATTCCCCCTTC
GGCTATCTCGATGTCCTTACGGAGAAGGAGATATTCGAGAGCTGTGTATGCAAGTTGATGGCCGCCAAGACGCGG
ATACTCGTCACGAGTAAGATGGAGCACCTCAAGAAGGCAGATAAGATTCTGATCCTTCATGAGGGCTCCTCTTAC
TTCTATGGGACGTTCAGTGAGCTGCAGAATCTTCAGCCCGATTTCTCCAGTAAGCTTATGGGCTGTGACTCCTTC
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGAGCTTTAAGCAGACTGGAGAATTCGGGGAGAAGAGAAAGAACAGC
ATACTGAACCCCATAGCTTCCATCCGGAAGTTCAGCATCGTCCAGAAGACTCCTCTCCAGATGAATGGAATCGAG
GAGGATTCGGACGAGCCCCTGGAGCGTCGGCTCAGTCTGGTCCCGGATTCCGAGCAGGGGGAGGCCATCCTCCCT
CGGATTTCGGTGATCAGCACTGGGCCGACGCTCCAGGCCCGGCGCCGTCAGAGCGTCCTGGCGCTGATGACACAC
AGCGTGGCGCAGGGTCAGGCGATCCATCGCAAGACCACTGCGTCCACGCGGAAGGTGTCATTAGCGCCACAGGCC
GCTCTGACGGAGCTGGATATCTACAGCCGCCGTCTCTCCCAGGAGACGGGGCTGGAGATATCGGAGGAGATCGCC
GAGGAGGACCTCAAGGAGTGCTTCTTCGACGACATGGAGAGCATTCCTGCGGTCACTACCTGGGCGACCTACCTC
CGATACATCACGGTACACAAGAGTTTGATATTCGTCTTGATATGGTGCCTGGTCATATTCCTAGCTGAAGTGGCT
GCATCGCTCGTGGTGTTGTGGCTGTTGGGTGCTACTCCCCTGCAGGACAAGGGGAATAGCACCCACAGCCGCAAC
GCCTCGTACGCGGTGATCATCACCAGTACGAGCTCCTACTATGTGTTCTACATATATGTAGGAGTTGCTGACACG
TTGTTAGCGATGGGCTTCTTCAGGGGGTTACCCCTGGTCCATACGCTGATAACGGTCAGCAAGATCCTGCACCAC
AAGATGCTCCATTCAGTGTTGCAAGCGCCGATGTCGACGCTTGCAACACTGAAGGCTGGAGGCATCTTGAACAGG
TTCTCGAAGGACATTGCAATACTGGACGACCTATTGCCCCTGACGATCTTCGACTTCATCCAGTTGTTGTTGATC
GTCATAGGGGCAATAGCGGTCGTCGCAGTATTGCAGCCGTACATCTTTGTCGCCACTGTGCCAGTTATCGTCGCC
TTCATAATGTTGAGAGCCTACTTCCTTCAGACTAGTCAGCAGCTGAAGCAGTTGGAGAGCGAGGGTCGCTCTCCA
ATCTTCACGCACTTGGTGACTAGTCTGAAGGGGCTCTGGACATTGCGGGCATTCGGGCGGCAGCCGTATTTTGAG
ACCTTGTTCCACAAGGCTCTCAATTTACACACTGCCGCCTGGTTCCTGTACCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGGCGAGTGGGGATCATTCTCACACTCGCCATGAACATCATGAGCACGCTCCAGTGGGCTGTCGCGTCGAGC
ATCGACGTCGACAGCCTCATGCGGAGCGTGTCTCGTGTGTTCAAGTTCATCGATATGCCTACGGAGGGCAAGCCC
ACCAAGAGTACGAAGCCGTACAAGAATGGGCAGCTCTCCAAGGTAATGATTATCGAGAACTCCCATGTGAAGAAG
GACGATATCTGGCCCAGTGGCGGCCAGATGACGGTTAAAGACCTTACTGCGAAGTACACCGAAGGCGGCAACGCC
ATTCTGGAGGCGATATCGTTTTCCATCTCACCCGGTCAGCGGGTCGGCCTCCTCGGTCGCACGGGGTCGGGGAAG
AGCACTCTGCTGAGTGCTTTTCTCCGACTCCTTGCGACCGAGGGGGAGATCCAGATTGACGGGGTGAGTTGGGAT
TCGATAACCCTCCAGCAGTGGCGTAAGGCCTTCGGTGTAATTCCGCAGAAGGTCTTTATCTTTTCCGGGACCTTC
CGGAAGGCTCTTGATCCGTACGAGCAATGGTCGGATCAAGAGATCTGGAAGGTCGCGGACGAAGTCGGGCTTCGC
TCGGTGATCGAGCAGTTCCCGGGCAAGCTCGACTTCGTCTTGGTCGATGGAGGGTGCGTTCTCTCGCACGGGCAC
AAGCAGCTGATGTGCCTGGCGAGGAGCGTACTCTCCAAGGCCAAGATCCTCCTCCTCGATGAGCCGTCGGCGCAT
CTGGACCCGGTGACTTACCAGATTATTCGCCGGACGCTGAAACAAGCTTTCGCGGACTGTACGGTGATATTGTGT
GAGCACCGGATAGAGGCTATGCTCGAGTGCCAGCAGTTTCTGGTTATCGAAGAGGCGAAGGTTCGCCAGTACGAT
AGCATCCAGAAGCTGCTGGCCGAGCGTAGCCTCTTCCGGCAGGCAATATCACCGTCAGACCGCGTAAAGCTGTTT
CCGCATCGGGCGAGTAGTAAGTGTAAGTCAAAGCCGCAGATCGCCGCGCTCAAGGAGGAGACGGAGGAGGAGGTG
CAGGACACGCGCCTCTAA
>CFTR_recode-6 (SEQ ID NO: 8)
ATGCAGAGGTCCCCCCTTGAGAAGGCATCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATCCAGGACTTCTTG
CAGAAGCAGGAGTATAAGACTCTGGAGTACGCCCTCACGACCACGGAGGTGGTGATGGAGAACGTCACCGCCTTC
TGGGAGGAGGGCTTCGGGGAGTTGTTCGAGAAGGCCAAGCAGGCCAACGCCAATCGAAAGACTTCGAATGGCGAT
GACAGCTTGTTCTTCTCGAACTTCTCCCTGCTGGGCACGCCGGTCTTGAAGGACATCAACTTCAAGATCGAGCGT
GGCCAGCTCCTGGCGGTGGCCGGGAGCACGGGGGCTGGGAAGACCAGCCTCCTCATGGTGATCATGGGGGAGCTC
GAGCCAAGTGAAGGGAAGATCAAACACTCGGGCCGGATTAGTTTTTGCTCGCAGTTTTCATGGATCATGCCCGGT
ACCATCAAGGAGAACATCATCTTTGGTGTCTCCTATGATGAGTACCGGTATCGATCCGTGATAAAGGCCTGCCAG
CTCGAGGAGGATATCTCCAAGTTCGCCGAGAAGGATAACATCGTTCTCGGCGAAGGAGGTATCACCTTGAGCGGC
GGGCAGCGAGCAAGAATTAGTCTGGCCCGAGCTGTTTACAAGGATGCTGACCTTTACTTGCTCGACTCCCCCTTT
GGTTACCTTGACGTACTCACAGAGAAGGAGATCTTTGAGTCCTGCGTCTGCAAGTTGATGGCGGCGAAGACCAGG
ATCTTGGTCACCTCTAAGATGGAGCATCTTAAGAAGGCGGACAAGATCCTCATTCTGCACGAGGGGAGCTCCTAC
TTCTATGGGACGTTCAGCGAGTTGCAGAATCTTCAGCCCGATTTCTCCAGTAAGCTTATGGGCTGCGATTCGTTT
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGTCCTTTAAGCAGACTGGAGAATTCGGGGAGAAGAGAAAGAACTCG
ATCCTGAACCCCATAGCAAGTATCAGGAAGTTCTCCATCGTGCAGAAGACGCCGCTCCAGATGAACGGAATCGAG
GAGGATTCCGACGAGCCTCTGGAGCGGCGTCTTTCCCTTGTTCCGGACTCGGAACAAGGGGAGGCCATACTGCCG
CGCATCAGCGTTATCAGCACGGGGCCGACGCTGCAGGCGCGGCGTCGGCAGTCCGTGCTGGCGCTGATGACGCAC
AGTGTGGCCCAGGGCCAGGCTATCCACCGCAAGACGACCGCGTCTACGCGGAAGGTTAGCCTGGCCCCCCAGGCG
GCACTGACGGAGCTCGACATATATTCGAGGAGGCTCTCCCAGGAGACCGGCTTGGAGATCTCGGAGGAGATCGCC
GAGGAGGATCTCAAGGAGTGCTTCTTCGATGATATGGAGAGCATTCCGGCAGTGACCACCTGGGCCACTTACCTT
CGCTACATCACCGTGCATAAGAGCTTGATATTCGTGCTGATCTGGTGTCTGGTTATCTTTCTCGCTGAAGTGGCG
GCGTCCCTAGTAGTCTTGTGGCTGCTAGGGGCGACGCCACTTCAGGACAAAGGTAACTCGACCCACAGCAGGAAT
GCAAGCTATGCGGTGATCATTACGAGCACCAGCTCGTACTACGTGTTCTACATCTACGTCGGGGTGGCGGACACG
CTGCTGGCCATGGGATTCTTCCGTGGCCTGCCGCTTGTCCACACCCTGATAACTGTTAGCAAGATCTTGCACCAC
AAGATGTTGCACAGTGTCCTTCAAGCGCCGATGAGTACTCTCGCCACGCTTAAAGCGGGCGGGATACTCAATCGG
TTCTCGAAGGACATCGCCATCCTAGACGATCTGCTGCCGCTGACGATATTCGACTTTATCCAGCTGCTGCTGATC
GTCATAGGGGCGATTGCGGTTGTAGCTGTGCTACAGCCGTATATATTCGTGGCCACGGTCCCAGTGATAGTGGCG
TTCATTATGTTGAGGGCGTACTTCCTTCAGACTTCCCAGCAGCTGAAGCAGCTGGAGTCTGAAGGAAGGTCGCCC
ATTTTCACCCACCTGGTGACAAGTCTCAAGGGCCTGTGGACGTTGAGGGCCTTTGGGAGGCAGCCGTACTTCGAG
ACGCTGTTCCATAAGGCCCTCAACCTCCACACGGCCGCTTGGTTCTTGTATCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGCCGGGTCGGGATCATCCTGACCCTGGCCATGAACATAATGAGCACCCTGCAGTGGGCCGTGGCCTCGAGT
ATAGATGTAGACTCACTGATGAGGAGCGTGTCGCGAGTTTTCAAGTTTATCGACATGCCTACGGAGGGCAAGCCC
ACCAAGAGTACGAAGCCGTACAAGAATGGGCAGCTCTCCAAGGTAATGATAATTGAGAACTCGCACGTCAAGAAA
GACGATATCTGGCCCTCCGGTGGGCAGATGACGGTCAAGGACCTCACTGCCAAGTACACGGAGGGCGGGAACGCC
ATTCTGGAAGCGATATCGTTTTCCATCTCACCCGGTCAGAGGGTCGGCCTCCTCGGTCGCACGGGGTCGGGGAAG
AGCACTCTGCTGAGTGCTTTTCTCCGACTCCTTGCGACCGAGGGGGAGATCCAGATCGACGGGGTGAGTTGGGAT
TCGATAACGCTCCAGCAGTGGCGTAAGGCCTTCGGGGTTATACCCCAGAAGGTCTTCATCTTTTCCGGGACCTTC
CGGAAGGCTCTTGATCCCTACGAGCAGTGGAGCGATCAAGAGATCTGGAAGGTCGCGGATGAGGTGGGGTTGAGG
AGCGTGATCGAGCAGTTTCCAGGAAAGCTGGACTTCGTCCTTGTTGACGGAGGCTGCGTGCTGAGCCATGGGCAC
AAGCAGCTCATGTGCTTAGCACGCAGCGTCCTCAGCAAGGCGAAGATCCTGCTCCTGGATGAGCCGAGCGCGCAC
CTCGACCCGGTGACGTACCAGATCATCCGGCGGACGTTGAAACAGGCCTTTGCGGACTGTACAGTGATCCTCTGC
GAGCATCGGATCGAGGCCATGCTGGAGTGCCAGCAGTTCCTCGTGATAGAGGAAGCGAAGGTACGCCAGTATGAC
TCGATCCAGAAGTTGCTCGCAGAGCGATCACTGTTCCGCCAGGCCATTTCACCGTCCGACCGGGTGAAGCTGTTC
CCGCACCGCGCCTCCTCCAAGTGTAAGTCAAAGCCGCAGATTGCGGCTTTGAAGGAGGAGACTGAGGAGGAGGTG
CAGGACACGCGCCTCTAA
>CFTR_recode-7 (SEQ ID NO: 9)
ATGCAGAGGTCCCCCCTTGAGAAGGCATCGGTCGTGTCCAAGCTGTTCTTCAGCTGGACACGACCCATCCTCCGG
AAGGGTTATCGGCAGCGACTGGAGCTATCTGATATCTATCAGATACCTTCAGTCGACTCTGCCGATAACCTCAGC
GAGAAGCTGGAGCGAGAATGGGATCGTGAGCTGGCGAGTAAGAAGGCCCCGAAGCTAATAGCTGCACTGCGTCGT
TGCTTCTTCTGGAGGTTTATGTTTTATGGCATATTCCTCTACTTAGGAGAAGTAACGAAGGCAGTGCAGCCGTTG
CTTCTGGGCCGGATTATCGCCAGCTACGATCCCGATGCGAAGGAGGAGAGGTCGATCGCCATCTACTTGGGGATC
GGCCTCTGCCTCCTTTTCATCGTGCGGACATTGTTGCTGCACCCGGCGATCTTCGGTCTGCACCACATCGGGATG
CAGATGAGGATCGCCATGTTCTCGCTGATCTACAAGAAGACGCTGAAGCTGAGCTCTCGCGTCTTGGACAAGATC
AGCATTGGGCAGCTGGTAAGCTTGCTCAGTGCTGCTCTTGCCAAGTTCGACGAGGGCTTAGCTTTAGCGCACTTC
GTGTGGATCGCCCCGCTGCAGGTGGCGCTCCTGATGGGGCTCATCTGGGAGCTCCTTCAGGCTAGCGCCTTCTGC
GGCTTGGGGTTCTTGATCGTCCTCGCATTATTTCAGGCGGGTCTCGGCCGCATGATGATGAAGTACCGGGACCAG
CGTGCCGGTAAGATCAGCGAGCGGCTGGTGATCACGTCTGAGATGATCGAGGCGATCCAGAGCGTTAAGGCGTAC
TGCTGGGAAGAAGCCATGGAGAAGATGATCGAGGCGCTGCGGCAAACCGAACTTAAGCTGACCAGAAAGGCAGCT
TATGTTCGGTACTTTGCCAGCAGCGCCTTCTTCTTCTCCGGCTTCTTCGTAGTATTCCTTAGCGTTCTGCCGTAC
GCGCTGATAAAGGGAATCATCCTCAGGAAGATCTTCACGACTATCAGCTTCTGCATCGTCTTACGAATGGCCGTC
ACGCGCCAGTTCCCATGGGCGGTGCAGACCTGGTATGACAGCCTCGGGGCGATAGCGAAGATCCAGGACTTCCTG
CAGAAGCAGGAGTACAAGACCCTGGAGTACGCGCTAACGACCACCGAGGTTGTCATGGAGGCTGTAACGGCCTTT
TGGGAAGAGGGGTTTGGCGAGCTATTCGAGAAGGCGAAGCAGGCAGCTGCTAATCGGAAGACTTCCAATGGGGAT
GATTCCCTTTTCTTCAGCGCGTTCAGTCTCTTGGGGACGCCGGTCCTCAAGGACATCGCGTTCAAGATTGAGCGC
GGCCAGCTTCTCGCTGTTGCCGGCTCCACCGGAGCCGGCAAAACCAGCTTGCTCATGGTGATAATGGGCGAGCTG
GAACCGTCCGAGGGCAAGATCAAGCACAGCGGGCGGATCAGCTTCTGCTCGCAGTTCAGCTGGATCATGCCCGGG
ACGATCAAGGAGGCAATAATCTTCGGGGTTAGCTACGACGAGTATAGATATCGCTCCGTCATCAAGGCCTGCCAG
CTCGAGGAGGATATCTCCAAGTTCGCCGAGAAGGATGCCATCGTTCTCGGCGAAGGAGGTATCACCTTGAGCGGT
GGCCAGAGAGCGCGTATTTCGCTGGCCAGAGCTGTATACAAGGACGCGGATCTCTACCTTCTCGACAGCCCGTTC
GGTTATCTGGACGTGCTGACCGAGAAGGAGATATTCGAGTCCTGTGTGTGCAAGCTCATGGCCGCGAAGACGCGC
ATTCTGGTCACCAGCAAGATGGAGCATCTGAAGAAGGCCGATAAGATACTGATTCTCCACGAGGGGAGTTCGTAT
TTTTACGGCACTTTTTCAGAGCTCCAGAACTTGCAGCCGGACTTCTCCAGTAAGCTTATGGGCTGCGATTCGTTT
GATCAGTTCTCCGCGGAGCGGCGGGCGTCCATCCTGACTGAGACGTTGCATCGTTTCAGTCTGGAGGGGGACGCC
CCCGTTTCGTGGACTGAGACGAAGAAGCAGTCCTTTAAGCAGACTGGAGAGTTCGGCGAGAAGCGAAAGGCGTCA
ATCTTGGCGCCTATCGCTTCTATTCGGAAGTTCAGCATCGTGCAGAAGACGCCGCTCCAGATGGCTGGTATCGAG
GAGGACTCGGACGAGCCTCTGGAGCGGCGTCTTTCGCTGGTGCCGGACTCCGAACAGGGCGAGGCTATCCTTCCG
CGCATCAGCGTTATCAGCACGGGGCCGACGCTGCAGGCGCGGCGTCGGCAGTCCGTGCTGGCGCTGATGACGCAT
AGCGTGGCGCAGGGTCAGGCGATCCATCGCAAGACCACTGCGTCCACGCGGAAGGTTAGCCTCGCCCCCCAGGCC
GCCTTGACGGAGCTTGATATCTATAGTCGTCGTTTGTCCCAGGAGACCGGTTTAGAGATCTCGGAGGAGATCGCG
GAGGAGGACTTGAAGGAGTGCTTCTTCGACGATATGGAGAGCATACCAGCAGTGACGACGTGGGCGACTTATCTA
CGGTACATAACCGTACATAAGTCGCTCATTTTCGTCCTGATCTGGTGTCTCGTCATATTTCTGGCGGAGGTGGCG
GCATCGCTAGTGGTGTTGTGGCTGCTGGGTGCTACCCCCTTGCAGGACAAGGGGGCTAGCACCCACAGCCGCAAC
GCCAGCTATGCGGTGATCATCACCTCCACCAGCTCCTACTATGTGTTCTACATATATGTAGGAGTTGCTGACACG
TTATTAGCGATGGGCTTCTTCAGGGGGTTACCCCTGGTCCATACGCTGATAACGGTCAGCAAAATCCTCCACCAT
AAGATGTTGCACAGCGTGCTCCAGGCCCCCATGAGCACGCTGGCAACTCTTAAGGCTGGAGGAATTCTGAACCGG
TTCTCCAAGGACATCGCTATCCTCGATGATTTATTGCCACTCACAATATTCGACTTCATCCAGTTGTTGTTGATC
GTCATAGGGGCGATTGCGGTTGTAGCTGTGCTACAGCCGTATATATTCGTGGCCACGGTCCCAGTGATAGTGGCG
TTCATTATGTTGAGGGCGTACTTCCTTCAGACTTCCCAGCAGCTGAAGCAGCTGGAGTCTGAAGGAAGGTCGCCC
ATTTTCACCCACCTGGTGACAAGTCTCAAGGGCCTGTGGACGTTGAGGGCCTTTGGGAGGCAGCCGTACTTCGAG
ACGCTGTTCCATAAGGCCCTCAACCTCCACACGGCCGCTTGGTTCTTGTATCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGCCGGGTCGGGATCATCCTGACCCTGGCCATGAACATAATGAGCACCCTGCAGTGGGCCGTGGCCTCGAGT
ATAGATGTAGACTCACTGATGAGGAGCGTGTCGCGAGTTTTCAAGTTTATCGACATGCCTACGGAGGGCAAGCCC
ACCAAGAGTACGAAGCCGTACAAGAATGGGCAGCTCTCCAAGGTAATGATAATTGAGAACTCGCACGTCAAGAAA
GACGATATCTGGCCCTCCGGTGGGCAGATGACGGTCAAGGACCTCACTGCCAAGTACACGGAGGGCGGGAACGCC
ATTCTGGAAGCGATATCGTTTTCCATCTCACCCGGTCAGAGGGTCGGCCTCCTCGGTCGCACGGGGTCGGGGAAG
AGCACTCTGCTGAGTGCTTTTCTCCGACTCCTTGCGACCGAGGGGGAGATCCAGATCGACGGGGTGAGTTGGGAT
TCGATAACGCTCCAGCAGTGGCGTAAGGCCTTCGGGGTTATACCCCAGAAGGTCTTCATCTTTTCCGGGACCTTC
CGGAAGGCTCTTGATCCCTACGAGCAGTGGAGCGATCAAGAGATCTGGAAGGTCGCGGATGAGGTGGGGTTGAGG
AGCGTGATCGAGCAGTTTCCAGGAAAGCTGGACTTCGTCCTTGTTGACGGAGGCTGCGTGCTGAGCCATGGGCAC
AAGCAGCTCATGTGCTTAGCACGCAGCGTCCTCAGCAAGGCGAAGATCCTGCTCCTGGATGAGCCGAGCGCGCAC
CTCGACCCGGTGACGTACCAGATCATCCGGCGGACGTTGAAACAGGCCTTTGCGGACTGTACAGTGATCCTCTGC
GAGCATCGGATCGAGGCCATGCTGGAGTGCCAGCAGTTCCTCGTGATAGAGGAAGCGAAGGTACGCCAGTATGAC
TCGATCCAGAAGTTGCTCGCAGAGCGATCACTGTTCCGCCAGGCCATTTCACCGTCCGACCGGGTGAAGCTGTTC
CCGCACCGCGCCTCCTCCAAGTGTAAGTCAAAGCCGCAGATTGCGGCTTTGAAGGAGGAGACTGAGGAGGAGGTG
CAGGACACGCGCCTCTAA
>CFTR_recode-8 (SEQ ID NO: 10)
ATGCAGAGGTCCCCCCTTGAGAAGGCATCGGTCGTGTCCAAGCTGTTCTTCAGCTGGACACGCCCGATACTCCGC
AAGGGCTACCGCCAGCGGCTGGAGCTCTCGGACATTTACCAGATCCCCTCCGTCGATTCCGCTGACGCTCTCAGC
GAGAAGCTGGAGCGTGAGTGGGATCGAGAGCTGGCGAGCAAGAAAGCGCCAAAATTAATAGCGGCGCTGCGCCGC
TGTTTTTTTTGGCGCTTTATGTTCTACGGCATCTTCCTGTATCTTGGCGAGGTAACGAAGGCGGTGCAGCCGCTC
CTCCTTGGCCGGATCATAGCCTCATATGATCCGGACGCCAAGGAGGAGCGGAGCATCGCCATCTACCTCGGCATC
GGTCTGTGTCTGCTGTTTATAGTGCGGACCTTATTGCTGCACCCAGCAATATTCGGTCTGCACCATATAGGCATG
CAGATGCGGATCGCGATGTTCTCGCTGATCTACAAGAAGACGCTGAAGCTGAGCTCTCGCGTCTTGGACAAGATC
AGCATTGGGCAGCTGGTAAGCTTGCTCAGTGCTGCTCTTGCCAAGTTCGACGAGGGCTTAGCTTTAGCGCACTTC
GTGTGGATCGCGCCGCTCCAGGTGGCCCTGTTAATGGGACTGATCTGGGAGCTGCTCCAGGCTTCCGCGTTCTGT
GGTCTCGGCTTCCTCATCGTCCTAGCCCTGTTTCAAGCAGGGCTAGGACGAATGATGATGAAGTATCGAGACCAG
AGAGCGGGGAAGATCTCTGAGCGGCTCGTGATCACGTCCGAGATGATAGAGGCCATCCAGAGCGTGAAAGCCTAC
TGTTGGGAGGAGGCTATGGAGAAGATGATCGAGGCGTTGCGGCAGACCGAGTTAAAGTTGACTCGGAAGGCCGCA
TACGTCCGATACTTCGCCTCCAGTGCCTTCTTCTTCAGTGGGTTTTTTGTCGTGTTTCTGTCTGTGCTGCCCTAT
GCATTGATAAAGGGGATCATCCTGAGGAAGATCTTCACGACGATCTCCTTTTGCATAGTGCTGCGCATGGCAGTG
ACACGACAGTTCCCCTGGGCTGTCCAGACATGGTACGACAGCCTAGGGGCAATCGCCAAGATACAGGACTTCCTG
CAGAAGCAGGAATACAAGACGCTGGAGTACGCCTTGACGACCACGGAGGTGGTCATGGAGGCGGTCACAGCGTTT
TGGGAGGAGGGATTTGGTGAATTGTTCGAGAAGGCCAAGCAGGCTGCAGCGAACCGGAAGACGAGCAACGGGGAC
GACTCGTTGTTCTTTTCCGCGTTCAGCCTGCTTGGCACGCCGGTCTTGAAGGACATCGCCTTCAAGATCGAGCGT
GGCCAGCTGCTGGCGGTAGCCGGGAGTACGGGCGCAGGGAAGACCAGCTTGCTCATGGTGATCATGGGCGAGCTG
GAGCCTTCCGAGGGCAAGATCAAGCACAGCGGGCGGATCAGCTTCTGCTCGCAGTTCAGCTGGATCATGCCCGGT
ACCATCAAGGAGGCTATCATCTTTGGTGTCTCCTATGATGAGTACCGATATCGGAGCGTGATCAAGGCCTGCCAG
CTCGAGGAGGATATCTCCAAGTTCGCCGAGAAGGACGCCATAGTCCTCGGCGAAGGAGGTATCACCTTGAGCGGC
GGGCAGCGCGCTCGGATATCGCTGGCCAGAGCTGTATACAAGGACGCGGATCTCTACCTTCTCGACAGCCCGTTC
GGTTATCTGGACGTGCTGACCGAGAAGGAGATATTCGAGTCCTGTGTATGCAAGCTCATGGCCGCGAAGACGCGG
ATATTGGTGACCAGTAAGATGGAGCACCTCAAGAAGGCGGACAAGATCCTTATCCTGCACGAGGGGTCGTCCTAC
TTCTACGGGACCTTCAGCGAGTTGCAGGCACTTCAGCCAGACTTTTCGTCCAAGCTGATGGGGTGCGACTCCTTC
GACCAGTTCTCGGCGGAGCGTCGTGCGTCAATCTTGACGGAGACGCTCCACCGGTTCTCGTTGGAGGGGGACGCA
CCCGTCAGCTGGACGGAGACGAAAAAACAGTCTTTTAAGCAGACTGGAGAATTTGGGGAGAAGCGAAAGGCGTCA
ATCTTGGCGCCTATCGCTTCTATCCGCAAATTCTCCATTGTGCAGAAGACGCCGCTCCAGATGGCTGGTATCGAG
GAGGACTCGGACGAGCCTCTGGAGCGGCGGCTTTCCCTTGTTCCGGACTCGGAGCAAGGGGAAGCCATCTTGCCG
CGGATTTCGGTGATATCGACCGGGCCCACGCTGCAAGCGAGGCGTCGTCAGAGCGTGCTGGCTCTGATGACGCAC
TCGGTTGCGCAGGGCCAGGCGATACACCGAAAGACGACGGCAAGTACCCGTAAGGTCAGCCTGGCCCCCCAGGCT
GCCCTTACGGAACTAGACATCTACTCAAGACGACTGAGTCAGGAGACTGGACTGGAGATATCGGAGGAGATCGCC
GAGGAGGACCTGAAGGAATGCTTCTTCGACGACATGGAGAGCATTCCTGCAGTGACCACCTGGGCGACCTACCTC
CGATATATCACAGTCCACAAGTCCCTGATTTTCGTCTTGATCTGGTGTCTAGTTATCTTCCTGGCTGAAGTGGCT
GCCTCGCTGGTGGTCCTGTGGTTGTTAGGGGCGACCCCTCTGCAGGATAAGGGTGCGTCCACCCACTCGAGGGCT
GCATCTTACGCGGTCATCATCACGTCCACGTCTTCGTATTATGTGTTCTACATATATGTCGGGGTGGCGGACACG
CTGCTGGCCATGGGATTCTTCCGTGGCCTGCCGCTTGTCCACACCCTGATAACCGTGTCCAAGATCCTGCACCAC
AAGATGTTGCACAGCGTGCTCCAGGCCCCCATGAGCACGCTGGCAACATTGAAGGCGGGCGGGATCTTGGCACGG
TTCAGCAAGGACATCGCGATCCTAGACGATCTGCTGCCGCTGACGATATTCGACTTTATCCAGCTGCTGCTGATC
GTCATAGGAGCCATCGCGGTCGTTGCTGTGCTGCAGCCATATATCTTTGTGGCTACAGTGCCGGTGATAGTCGCA
TTCATCATGCTGCGAGCATATTTCCTCCAGACTTCCCAGCAACTGAAGCAGTTAGAGAGCGAAGGCCGATCTCCC
ATCTTCACTCACCTGGTCACGTCGCTCAAAGGCCTGTGGACCCTACGGGCCTTTGGGCGACAGCCTTATTTCGAG
ACCCTCTTCCATAAGGCGCTGGCCCTTCATACGGCTGCGTGGTTTTTGTACCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGGCGAGTCGGCATCATATTGACGCTGGCCATGGCCATTATGAGTACTCTCCAATGGGCCGTGGCCAGCTCA
ATAGATGTCGACTCGCTCATGCGTTCCGTCAGTCGCGTGTTTAAGTTCATAGACATGCCGACTGAGGGAAAGCCC
ACAAAATCCACGAAGCCGTATAAGAACGGCCAGCTGTCCAAGGTGATGATTATTGAAGCTAGTCACGTCAAGAAG
GACGACATCTGGCCCTCCGGTGGGCAGATGACGGTGAAGGACCTCACTGCCAAGTACACGGAGGGCGGGGCCGCC
ATTCTTGAGGCGATTAGCTTCAGTATATCACCTGGACAGAGGGTGGGCTTGCTGGGCCGGACCGGGTCCGGCAAG
TCCACCCTCCTCTCCGCCTTCCTTCGCTTGCTTGCGACGGAAGGGGAGATCCAGATCGATGGGGTGTCCTGGGAT
TCCATAACGTTGCAGCAGTGGAGGAAGGCCTTCGGGGTTATACCCCAGAAGGTCTTCATCTTCTCTGGGACGTTT
CGGAAGGCCCTGGACCCCTACGAGCAGTGGAGTGACCAGGAGATCTGGAAGGTGGCCGACGAGGTCGGCCTTCGC
TCTGTGATTGAGCAGTTTCCTGGGAAGCTGGATTTTGTGCTCGTAGATGGTGGATGCGTACTATCACACGGCCAC
AAGCAGTTGATGTGCCTCGCTCGCTCCGTCTTGTCCAAGGCCAAGATCCTGCTGCTGGACGAGCCCAGCGCTCAC
CTCGATCCGGTGACGTACCAGATCATCCGGCGAACGCTGAAGCAAGCGTTCGCCGACTGCACGGTGATCTTGTGC
GAGCACCGGATCGAGGCGATGCTGGAGTGCCAGCAGTTCTTGGTCATCGAGGAGGCCAAGGTGCGGCAGTACGAC
TCGATCCAGAAGCTTCTGGCGGAGAGAAGCCTGTTCCGGCAGGCTATCTCTCCGTCAGATCGGGTCAAGTTGTTT
CCGCACCGGGCCTCCTCGAAATGCAAGTCAAAGCCGCAGATTGCGGCTTTGAAGGAGGAGACGGAGGAGGAGGTA
CAGGATACTCGCTTGTGA
>CFTR_recode-9 (SEQ ID NO: 11)
ATGCAGAGGTCCCCCCTTGAGAAGGCATCGGTCGTGTCCAAGCTGTTCTTCAGCTGGACACGCCCAATTCTTCGG
AAAGGTTATCGGCAGCGACTGGAGTTATCTGATATCTATCAGATACCTTCAGTCGACTCTGCCGATAACCTTTCC
GAAAAGTTGGAGCGCGAATGGGACCGCGAGCTCGCGTCCAAGAAGAACCCGAAGCTCATAAACGCGCTCAGAAGA
TGCTTCTTCTGGCGCTTTATGTTCTACGGGATCTTCCTGTATCTTGGCGAGGTCACCAAGGCTGTCCAGCCGCTC
CTCCTTGGCCGGATCATAGCCTCATATGATCCGGACGCCAAGGAGGAGCGGAGCATTGCTATATACCTGGGCATA
GGCCTGTGCCTCCTCTTCATAGTTCGGACTCTGCTGCTGCACCCGGCCATCTTCGGCCTCCACCACATCGGTATG
CAGATGCGGATTGCGATGTTCTCTCTGATTTATAAGAAGACGCTCAAGCTCAGCTCGCGGGTCCTGGACAAGATC
TCAATCGGCCAACTGGTCAGCCTGTTGAGCGCAGCACTAGCCAAGTTCGACGAAGGGCTGGCGCTTGCGCATTTC
GTATGGATAGCCCCATTGCAGGTGGCACTGCTAATGGGGCTAATATGGGAATTGCTGCAAGCGTCAGCCTTTTGC
GGACTTGGCTTCCTGATAGTGCTGGCGCTCTTCCAGGCTGGCCTTGGCCGAATGATGATGAAGTACCGGGACCAG
CGAGCTGGGAAGATCTCGGAGCGTCTTGTTATAACATCAGAGATGATCGAGAACATCCAATCCGTAAAGGCATAC
TGTTGGGAGGAGGCCATGGAGAAGATGATCGAGGCGCTGCGGCAGACCGAACTAAAGTTGACGAGGAAGGCAGCC
TATGTCCGGTATTTCAATAGCAGTGCTTTCTTCTTCTCGGGGTTCTTTGTGGTGTTCTTGTCAGTGCTGCCCTAT
GCACTGATCAAGGGCATCATACTCCGGAAGATTTTTACCACGATCAGCTTCTGCATCGTCCTACGGATGGCGGTG
ACCCGCCAGTTTCCGTGGGCGGTGCAGACGTGGTACGACAGCCTTGGTGCCATCGCCAAGATACAGGACTTCCTG
CAGAAGCAGGAATACAAGACGCTGGAGTACGCCTTGACGACCACGGAGGTGGTCATGGAGGCGGTCACAGCGTTT
TGGGAGGAGGGATTTGGTGAATTGTTCGAGAAGGCCAAGCAGGCTGCAGCGAACCGGAAGACGAGCAACGGGGAC
GACTCGTTGTTCTTTTCCGCGTTCAGCCTGCTTGGCACGCCGGTCTTGAAGGACATCGCCTTCAAGATCGAGCGT
GGCCAGCTGCTGGCGGTAGCCGGGAGTACGGGCGCAGGGAAGACCAGCTTGCTCATGGTGATCATGGGCGAGCTG
GAGCCTTCCGAGGGCAAGATCAAGCACAGCGGGCGGATCAGCTTCTGCTCGCAGTTCAGCTGGATCATGCCCGGT
ACCATCAAGGAGGCTATCATCTTTGGTGTCTCCTATGATGAGTACCGATATCGGAGCGTGATCAAGGCCTGCCAG
CTCGAGGAGGATATCTCCAAGTTCGCCGAGAAGGACGCCATAGTCCTCGGCGAAGGAGGTATCACCTTGAGCGGC
GGGCAGCGCGCTCGGATATCGCTGGCCAGAGCTGTATACAAGGACGCGGATCTCTACCTTCTCGACAGCCCGTTC
GGTTATCTGGACGTGCTGACCGAGAAGGAGATATTCGAGTCCTGTGTATGCAAGCTCATGGCCGCGAAGACGCGG
ATATTGGTGACCAGTAAGATGGAGCACCTCAAGAAGGCGGACAAGATCCTTATCCTGCACGAGGGGTCGTCCTAC
TTCTACGGGACCTTCAGCGAGTTGCAGGCACTTCAGCCAGACTTTTCGTCCAAGCTGATGGGGTGCGACTCCTTC
GACCAGTTCTCGGCGGAGCGTCGTGCGTCAATCTTGACGGAGACGCTCCACCGGTTCTCGTTGGAGGGGGACGCA
CCCGTCAGCTGGACGGAGACGAAAAAACAGTCTTTTAAGCAGACTGGAGAATTTGGGGAGAAGCGAAAGGCGTCA
ATCTTGGCGCCTATCGCTTCTATCCGCAAATTCTCCATTGTGCAGAAGACGCCGCTCCAGATGGCTGGTATCGAG
GAGGACTCGGACGAGCCTCTGGAGCGGCGGCTTTCCCTTGTTCCGGACTCGGAGCAAGGGGAAGCCATCTTGCCG
CGGATTTCGGTGATATCGACCGGGCCCACGCTGCAAGCGAGGCGTCGTCAGAGCGTGCTGGCTCTGATGACGCAC
TCGGTTGCGCAGGGCCAGGCGATACACCGAAAGACGACGGCAAGTACCCGTAAGGTCAGCCTGGCCCCCCAGGCT
GCCCTTACGGAACTAGACATCTACTCAAGACGACTGAGTCAGGAGACTGGACTGGAGATATCGGAGGAGATCGCC
GAGGAGGACCTGAAGGAATGCTTCTTCGACGACATGGAGAGCATTCCTGCAGTGACCACCTGGGCGACCTACCTC
CGATATATCACAGTCCACAAGTCCCTGATTTTCGTCTTGATCTGGTGTCTAGTTATCTTCCTGGCTGAAGTGGCT
GCCTCGCTGGTGGTCCTGTGGTTGTTAGGGGCGACCCCTCTGCAGGATAAGGGTGCGTCCACCCACTCGAGGGCT
GCATCTTACGCGGTCATCATCACGTCCACGTCTTCGTATTATGTGTTCTACATATATGTCGGGGTGGCGGACACG
CTGCTGGCCATGGGATTCTTCCGTGGCCTGCCGCTTGTCCACACCCTGATAACCGTGTCCAAGATCCTGCACCAC
AAGATGTTGCACAGCGTGCTCCAGGCCCCCATGAGCACGCTGGCAACATTGAAGGCGGGCGGGATCTTGGCACGG
TTCAGCAAGGACATCGCGATCCTAGACGATCTGCTGCCGCTGACGATATTCGACTTTATCCAGCTGCTGCTGATC
GTCATAGGAGCCATCGCGGTCGTTGCTGTGCTGCAGCCATATATCTTTGTGGCTACAGTGCCGGTGATAGTCGCA
TTCATCATGCTGCGAGCATATTTCCTCCAGACTTCCCAGCAACTGAAGCAGTTAGAGAGCGAAGGCCGATCTCCC
ATCTTCACTCACCTGGTCACGTCGCTCAAAGGCCTGTGGACCCTACGGGCCTTTGGGCGACAGCCTTATTTCGAG
ACCCTCTTCCATAAGGCGCTGGCCCTTCATACGGCTGCGTGGTTTTTGTACCTCTCCACTCTCCGGTGGTTCCAG
ATGAGGATCGAGATGATCTTCGTGATATTCTTTATCGCGGTGACCTTCATCTCGATCCTCACCACCGGAGAGGGA
GAGGGGCGAGTCGGCATCATATTGACGCTGGCCATGGCCATTATGAGTACTCTCCAATGGGCCGTGGCCAGCTCA
ATAGATGTCGACTCGCTCATGCGTTCCGTCAGTCGCGTGTTTAAGTTCATAGACATGCCGACTGAGGGAAAGCCC
ACAAAATCCACGAAGCCGTATAAGAACGGCCAGCTGTCCAAGGTGATGATTATTGAAGCTAGTCACGTCAAGAAG
GACGACATCTGGCCCTCCGGTGGGCAGATGACGGTGAAGGACCTCACTGCCAAGTACACGGAGGGCGGGGCCGCC
ATTCTTGAGGCGATTAGCTTCAGTATATCACCTGGACAGAGGGTGGGCTTGCTGGGCCGGACCGGGTCCGGCAAG
TCCACCCTCCTCTCCGCCTTCCTTCGCTTGCTTGCGACGGAAGGGGAGATCCAGATCGATGGGGTGTCCTGGGAT
TCCATAACGTTGCAGCAGTGGAGGAAGGCCTTCGGGGTTATACCCCAGAAGGTCTTCATCTTCTCTGGGACGTTT
CGGAAGGCCCTGGACCCCTACGAGCAGTGGAGTGACCAGGAGATCTGGAAGGTGGCCGACGAGGTCGGCCTTCGC
TCTGTGATTGAGCAGTTTCCTGGGAAGCTGGATTTTGTGCTCGTAGATGGTGGATGCGTACTATCACACGGCCAC
AAGCAGTTGATGTGCCTCGCTCGCTCCGTCTTGTCCAAGGCCAAGATCCTGCTGCTGGACGAGCCCAGCGCTCAC
CTCGATCCGGTGACGTACCAGATCATCCGGCGAACGCTGAAGCAAGCGTTCGCCGACTGCACGGTGATCTTGTGC
GAGCACCGGATCGAGGCGATGCTGGAGTGCCAGCAGTTCTTGGTCATCGAGGAGGCCAAGGTGCGGCAGTACGAC
TCGATCCAGAAGCTTCTGGCGGAGAGAAGCCTGTTCCGGCAGGCTATCTCTCCGTCAGATCGGGTCAAGTTGTTT
CCGCACCGGGCCTCCTCGAAATGCAAGTCAAAGCCGCAGATTGCGGCTTTGAAGGAGGAGACGGAGGAGGAGGTA
CAGGATACTCGCTTGTGA
>CFTR_recode-10 (SEQ ID NO: 12)
ATGCAGAGGTCCCCCCTTGAGAAGGCATCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATCCAGGACTTCTTG
CAGAAGCAGGAGTATAAGACTCTGGAGTACGCCCTCACGACCACGGAGGTGGTGATGGAGAACGTCACCGCCTTC
TGGGAGGAGGGCTTCGGGGAGTTGTTCGAGAAGGCCAAGCAGGCCAACGCCAATCGAAAGACTTCGAATGGCGAT
GACAGCTTGTTCTTCTCGAACTTCTCCCTGCTGGGCACGCCGGTCTTGAAGGACATCAACTTCAAGATCGAGCGT
GGCCAGCTCCTGGCGGTGGCCGGGAGCACGGGGGCTGGGAAGACCAGCCTCCTCATGGTGATCATGGGGGAGCTC
GAGCCAAGTGAAGGGAAGATCAAACACTCGGGCCGGATTAGTTTTTGCTCGCAGTTTTCATGGATCATGCCCGGT
ACCATCAAGGAGAACATCATCTTTGGTGTCTCCTATGATGAGTACCGGTATCGATCCGTGATAAAGGCCTGCCAG
CTCGAGGAGGATATCTCCAAGTTCGCCGAGAAGGATAACATCGTTCTCGGCGAAGGAGGTATCACCTTGAGCGGC
GGGCAGCGAGCAAGAATTAGTCTGGCCCGAGCTGTTTACAAGGATGCTGACCTTTACTTGCTCGACTCCCCCTTT
GGTTACCTTGACGTACTCACAGAGAAGGAGATCTTTGAGTCCTGCGTCTGCAAGCTGATGGCGGCCAAGACCAGG
ATCTTGGTCACCTCTAAGATGGAGCATCTTAAGAAGGCGGACAAGATCCTCATTCTGCACGAGGGGAGCTCCTAC
TTCTATGGGACGTTCAGCGAGTTGCAGAATCTTCAGCCCGATTTCTCCAGTAAGCTTATGGGCTGCGATTCGTTT
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGTCCTTTAAGCAGACTGGAGAATTCGGGGAGAAGAGAAAGAACTCG
ATCCTGAACCCCATAGCAAGTATCAGGAAGTTCTCCATCGTGCAGAAAACGCCGCTCCAGATGAACGGAATCGAG
GAGGATTCCGACGAGCCTCTGGAGCGGCGGCTTTCCCTTGTTCCGGACTCGGAACAAGGGGAAGCCATCTTGCCG
CGCATCAGCGTGATCAGCACGGGGCCGACGCTGCAGGCGCGGCGTCGGCAGTCCGTGCTGGCTCTGATGACGCAC
TCTGTGGCGCAAGGGCAGGCCATCCATCGTAAGACGACCGCGTCTACGAGGAAGGTCTCCCTTGCGCCACAGGCT
GCGTTAACAGAGCTGGATATCTACAGCCGCCGTCTCTCCCAGGAGACGGGGCTGGAGATATCCGAAGAGATCGCG
GAGGAGGACTTGAAGGAGTGCTTCTTCGACGATATGGAGTCCATTCCGGCCGTGACCACATGGGCGACTTATCTA
CGGTATATTACCGTACATAAGTCATTAATTTTCGTTTTGATATGGTGTTTAGTTATTTTCCTTGCCGAAGTGGCG
GCGTCCCTAGTAGTCTTGTGGCTACTAGGGGCGACGCCACTTCAGGACAAGGGAAATAGCACACATAGTAGGAAC
GCGTCGTACGCGGTGATAATCACGTCGACGAGTTCCTACTATGTGTTCTACATCTATGTGGGGGTTGCTGACACG
TTGTTAGCGATGGGCTTCTTCAGGGGGTTACCCCTGGTCCATACGCTGATAACGGTCAGCAAGATCCTCCACCAT
AAGATGTTACACTCGGTGCTTCAAGCACCGATGAGTACGCTCGCAACATTGAAGGCTGGCGGTATCCTGAACCGT
TTCAGCAAAGATATTGCAATACTGGACGACCTGTTGCCCCTGACGATCTTCGACTTCATCCAGTTGTTGTTGATC
GTCATAGGGGCAATAGCGGTCGTCGCAGTGTTGCAGCCCTATATCTTTGTTGCGACGGTTCCGGTTATCGTCGCC
TTCATAATGTTGCGAGCGTACTTCCTCCAGACGTCCCAGCAGCTGAAGCAACTAGAGAGTGAAGGTCGGTCCCCG
ATCTTCACTCATCTAGTTACTTCGCTGAAGGGACTCTGGACGCTGAGGGCATTCGGCAGGCAGCCGTATTTTGAG
ACGCTTTTCCATAAAGCGCTCAATTTACACACGGCTGCCTGGTTCTTGTACCTCAGCACGTTACGGTGGTTCCAG
ATGAGGATAGAGATGATCTTTGTCATCTTCTTTATCGCCGTGACGTTCATCTCCATCCTCACCACCGGTGAGGGG
GAGGGCCGGGTCGGGATCATCCTGACCCTGGCCATGAACATCATGTCAACCCTTCAGTGGGCTGTCGCGAGCTCG
ATAGACGTCGACTCACTGATGCGTTCAGTGAGTCGCGTCTTCAAGTTCATCGACATGCCCACTGAAGGGAAACCT
ACGAAGAGCACCAAGCCGTACAAGAACGGCCAGCTCTCGAAGGTAATGATTATTGAGAACAGCCACGTGAAGAAG
GACGATATATGGCCTTCAGGAGGGCAGATGACAGTGAAGGACCTCACTGCCAAGTACACCGAGGGGGGCAACGCG
ATCCTCGAGGCGATATCGTTCTCGATATCGCCCGGGCAGCGCGTTGGCCTCCTCGGTCGTACTGGCAGTGGGAAG
TCCACACTGTTATCTGCCTTCCTGCGCCTTCTCGCCACTGAGGGAGAGATCCAGATTGATGGAGTCTCCTGGGAC
TCCATCACTCTCCAGCAGTGGCGGAAGGCGTTCGGTGTCATTCCGCAGAAGGTGTTCATCTTTTCGGGGACCTTC
CGGAAGGCTCTTGATCCCTACGAGCAGTGGAGCGATCAAGAGATCTGGAAGGTCGCCGATGAGGTGGGCCTGCGG
AGTGTCATCGAGCAGTTCCCCGGGAAGCTGGACTTCGTCCTTGTTGACGGAGGCTGCGTGCTGAGCCATGGGCAC
AAGCAGCTCATGTGCTTAGCACGCAGCGTCCTCAGCAAGGCGAAGATCCTGCTTCTCGATGAGCCTTCAGCTCAC
CTCGATCCGGTGACGTACCAGATCATTCGGCGAACGCTGAAGCAAGCGTTCGCCGACTGCACTGTGATCTTGTGC
GAGCACCGGATCGAGGCGATGCTGGAGTGCCAGCAGTTCCTGGTAATCGAAGAAGCGAAGGTTCGGCAGTACGAC
AGCATCCAGAAGCTGTTGGCCGAGCGTTCGCTTTTTCGACAGGCCATATCGCCTTCTGATCGCGTGAAGCTGTTC
CCGCACCGGGCCTCCTCCAAGTGTAAGTCAAAGCCGCAGATTGCGGCTTTGAAGGAGGAGACTGAGGAGGAGGTC
CAGGACACGCGGCTCTAA
>CFTR_recode-11 (SEQ ID NO: 13)
ATGCAGAGGTCCCCCCTTGAGAAGGCATCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATCCAGGACTTCTTG
CAGAAGCAGGAGTATAAGACTCTGGAGTACGCCCTCACGACCACGGAGGTGGTGATGGAGAACGTCACCGCCTTC
TGGGAGGAGGGCTTCGGGGAGTTGTTCGAGAAGGCCAAGCAGGCCAACGCCAATCGAAAGACTTCGAATGGCGAT
GACAGCTTGTTCTTCTCGAACTTCTCCCTGCTGGGCACGCCGGTCTTGAAGGACATCAACTTCAAGATCGAGCGT
GGCCAGCTCCTGGCGGTGGCCGGGAGCACGGGGGCTGGGAAGACCAGCCTCCTCATGGTGATCATGGGGGAGCTC
GAGCCAAGTGAAGGGAAGATCAAACACTCGGGCCGGATTAGTTTTTGCTCGCAGTTTTCATGGATCATGCCCGGT
ACCATCAAGGAGAACATCATCTTTGGTGTCTCCTATGATGAGTACCGGTATCGATCCGTGATAAAGGCCTGCCAG
CTCGAGGAGGATATCTCCAAGTTCGCCGAGAAGGATAACATCGTTCTCGGCGAAGGAGGTATCACCTTGAGCGGC
GGGCAGCGAGCAAGAATTAGTCTGGCCCGAGCTGTTTACAAGGATGCTGACCTTTACTTGCTCGACTCCCCCTTT
GGTTACCTTGACGTACTCACAGAGAAGGAGATCTTTGAGTCCTGCGTCTGCAAGCTGATGGCGGCCAAGACCAGG
ATCTTGGTCACCTCTAAGATGGAGCATCTTAAGAAGGCGGACAAGATCCTCATTCTGCACGAGGGGAGCTCCTAC
TTCTATGGGACGTTCAGCGAGTTGCAGAATCTTCAGCCCGATTTCTCCAGTAAGCTTATGGGCTGCGATTCGTTT
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGTCCTTTAAGCAGACTGGAGAATTCGGGGAGAAGAGAAAGAACTCG
ATCCTGAACCCCATAGCAAGTATCAGGAAGTTCTCCATCGTGCAGAAAACGCCGCTCCAGATGAACGGAATCGAG
GAGGATTCCGACGAGCCTCTGGAGCGGCGGCTTTCCCTTGTTCCGGACTCGGAACAAGGGGAAGCCATCTTGCCG
CGCATCAGCGTGATCAGCACGGGGCCGACGCTGCAGGCGCGGCGTCGGCAGTCCGTGCTGGCTCTGATGACGCAC
TCTGTGGCGCAAGGGCAGGCCATCCATCGTAAGACGACCGCGTCTACGAGGAAGGTCTCCCTTGCGCCACAGGCT
GCGTTAACAGAGCTGGATATCTACAGCCGCCGTCTCTCCCAGGAGACGGGGCTGGAGATATCCGAAGAGATCGCG
GAGGAGGACTTGAAGGAGTGCTTCTTCGACGATATGGAGTCCATTCCGGCCGTGACCACATGGGCGACATACCTG
CGGTATATTACTGTCCACAAGAGCCTCATCTTTGTCTTGATATGGTGTTTAGTTATTTTCCTTGCCGAAGTGGCG
GCGTCCCTAGTAGTCTTGTGGCTACTAGGGGCGACGCCACTTCAGGACAAGGGAAATAGCACACATAGTAGGAAC
GCGTCGTACGCGGTGATAATCACGTCGACGAGTTCCTACTATGTGTTCTACATCTATGTGGGGGTTGCTGACACG
TTGTTAGCGATGGGCTTCTTCAGGGGGTTACCCCTGGTCCATACGCTGATAACGGTCAGCAAGATCCTCCACCAT
AAGATGTTACACTCGGTGCTTCAAGCACCGATGAGTACGCTCGCAACATTGAAGGCTGGCGGTATCCTGAACCGT
TTCAGCAAAGATATTGCAATACTGGACGACCTGTTGCCCCTGACGATCTTCGACTTCATCCAGTTGTTGTTGATC
GTCATAGGGGCAATAGCGGTCGTCGCAGTGTTGCAGCCCTATATCTTTGTTGCGACGGTTCCGGTTATCGTCGCC
TTCATAATGTTGCGAGCGTACTTCCTCCAGACGTCCCAGCAGCTGAAGCAACTAGAGAGTGAAGGTCGGTCCCCG
ATCTTCACTCATCTAGTTACTTCGCTGAAGGGACTCTGGACGCTGAGGGCATTCGGCAGGCAGCCGTATTTTGAG
ACGCTTTTCCATAAAGCGCTCAATTTACACACGGCTGCCTGGTTCTTGTACCTCAGCACGTTACGGTGGTTCCAG
ATGAGGATAGAGATGATCTTTGTCATCTTCTTTATCGCCGTGACGTTCATCTCCATCCTCACCACCGGTGAGGGG
GAGGGCCGGGTCGGGATCATCCTGACCCTGGCCATGAACATCATGTCAACCCTTCAGTGGGCTGTCGCGAGCTCG
ATAGACGTCGACTCACTGATGCGTTCAGTGAGTCGCGTCTTCAAGTTCATCGACATGCCCACTGAAGGGAAGCCC
ACCAAGAGTACGAAGCCGTACAAGAATGGGCAGCTGTCCAAGGTGATGATTATCGAGAATAGTCACGTCAAGAAG
GACGACATCTGGCCCTCCGGCGGTCAGATGACCGTGAAGGATCTGACCGCCAAGTACACGGAGGGCGGGAACGCC
ATTCTTGAGGCGATTAGCTTCTCGATATCACCTGGACAGCGGGTCGGCCTCCTCGGTCGCACGGGGTCGGGGAAG
AGCACTCTGCTGAGTGCTTTTCTCCGACTCCTTGCGACCGAGGGGGAGATCCAGATAGATGGGGTCTCTTGGGAC
AGTATTACGCTGCAGCAGTGGAGGAAGGCCTTCGGGGTTATACCCCAGAAGGTCTTCATATTCTCCGGGACCTTC
CGGAAGGCTCTTGATCCCTACGAGCAGTGGAGCGATCAAGAGATCTGGAAGGTCGCGGACGAAGTTGGACTGCGC
TCGGTGATAGAGCAGTTCCCCGGGAAGTTGGACTTTGTCTTGGTCGATGGAGGGTGCGTTCTGAGCCACGGCCAT
AAGCAGCTTATGTGCCTGGCTCGGAGCGTACTCTCCAAGGCCAAGATATTGCTGCTGGACGAGCCCAGCGCTCAC
CTCGATCCGGTGACGTACCAGATCATTCGGCGAACGCTGAAGCAAGCGTTCGCCGACTGCACTGTGATCTTGTGC
GAGCACCGGATCGAGGCGATGCTGGAGTGCCAGCAGTTCCTGGTGATCGAAGAGGCTAAGGTCCGCCAGTATGAT
TCGATCCAGAAGTTACTGGCGGAGCGTAGCCTCTTCCGCCAGGCCATCAGTCCAAGTGACCGGGTGAAGCTGTTC
CCTCACCGAGCGTCGTCCAAGTGTAAGTCAAAGCCGCAGATTGCGGCTTTGAAAGAAGAAACAGAGGAGGAGGTC
CAGGACACCCGCCTCTGA
>CFTR_recode-12 (SEQ ID NO: 14)
ATGCAGAGATCGCCGCTGGAGAAGGCTTCGGTCGTGTCCAAGCTGTTCTTCAGCTGGACACGACCGATCCTTCGC
AAGGGGTACCGCCAGCGACTGGAGCTATCTGATATCTATCAGATACCTTCAGTCGACTCGGCGGATAATCTGTCC
GAGAAGCTAGAGCGTGAATGGGATCGTGAGCTCGCCAGCAAGAAGAACCCGAAGCTCATAAACGCGCTCAGAAGA
TGCTTCTTCTGGCGCTTTATGTTCTACGGGATCTTCTTGTACCTTGGCGAGGTCACGAAGGCCGTTCAGCCTCTG
CTTCTCGGACGGATTATCGCTAGTTATGACCCCGATGCTAAGGAGGAGCGGTCCATCGCGATATACTTAGGCATA
GGCTTATGCCTATTGTTTATCGTGAGGACCCTCCTCCTGCATCCGGCGATCTTCGGTCTGCACCACATCGGGATG
CAGATGAGGATCGCCATGTTTTCTTTGATCTATAAGAAGACATTGAAGTTGTCGAGCCGGGTCCTCGACAAAATT
AGCATAGGGCAGCTGGTGAGCTTATTATCAGCTGCCCTAGCTAAGTTTGACGAGGGCCTGGCTCTGGCACACTTC
GTCTGGATCGCCCCATTGCAGGTGGCACTGCTAATGGGGCTGATCTGGGAGCTGCTCCAGGCTTCCGCGTTCTGT
GGTCTCGGATTTCTCATCGTTCTGGCCCTGTTCCAGGCGGGACTGGGCCGGATGATGATGAAGTACCGAGACCAG
AGAGCGGGGAAGATCTCGGAGCGGCTGGTCATAACTAGCGAGATGATCGAGAACATTCAGAGCGTGAAGGCGTAC
TGCTGGGAGGAGGCCATGGAGAAGATGATTGAAGCATTAAGGCAGACGGAGTTGAAGCTGACACGTAAGGCCGCT
TACGTGCGGTACTTCAACTCCTCTGCCTTTTTCTTCAGTGGCTTCTTCGTGGTCTTCCTCAGCGTACTGCCTTAC
GCTCTGATCAAGGGGATCATCCTTCGCAAGATCTTCACGACTATCAGCTTCTGCATCGTCCTACGGATGGCGGTG
ACCCGCCAGTTTCCGTGGGCGGTGCAGACCTGGTATGATAGTCTTGGAGCCATTGCGAAGATCCAGGACTTCTTG
CAGAAGCAGGAGTATAAGACTCTGGAGTACGCCCTCACGACCACGGAGGTGGTGATGGAGAACGTCACCGCCTTC
TGGGAGGAGGGCTTCGGGGAGTTGTTCGAGAAGGCCAAGCAGGCCAACGCCAATCGAAAGACTTCGAATGGCGAT
GACAGCTTGTTCTTCTCGAACTTCTCCCTGCTGGGCACGCCGGTCTTGAAGGACATCAACTTCAAGATCGAGCGT
GGCCAGCTCCTGGCGGTGGCCGGGAGCACGGGGGCTGGGAAGACCAGCCTCCTCATGGTGATCATGGGGGAGCTC
GAGCCAAGTGAAGGGAAGATCAAACACTCGGGCCGGATTAGTTTTTGCTCGCAGTTTTCATGGATCATGCCCGGT
ACCATCAAGGAGAACATCATCTTTGGTGTCTCCTATGATGAGTACCGGTATCGATCCGTGATAAAGGCCTGCCAG
CTCGAGGAGGATATCTCCAAGTTCGCCGAGAAGGATAACATCGTTCTCGGCGAAGGAGGTATCACCTTGAGCGGC
GGGCAGCGAGCAAGAATTAGTCTGGCCCGAGCTGTTTACAAGGATGCTGACCTTTACTTGCTCGACTCCCCCTTT
GGTTACCTTGACGTACTCACAGAGAAGGAGATCTTTGAGTCCTGCGTCTGCAAGCTGATGGCGGCCAAGACCAGG
ATCTTGGTCACCTCTAAGATGGAGCATCTTAAGAAGGCGGACAAGATCCTCATTCTGCACGAGGGGAGCTCCTAC
TTCTATGGGACGTTCAGCGAGTTGCAGAATCTTCAGCCCGATTTCTCCAGTAAGCTTATGGGCTGCGATTCGTTT
GATCAGTTCTCAGCTGAGCGGCGCAACTCCATTCTGACGGAGACCTTGCATAGGTTTTCGTTAGAAGGAGATGCG
CCGGTCAGCTGGACTGAGACGAAGAAGCAGTCCTTTAAGCAGACTGGAGAATTCGGGGAGAAGAGAAAGAACTCG
ATCCTGAACCCCATAGCAAGTATCAGGAAGTTCTCCATCGTGCAGAAAACGCCGCTCCAGATGAACGGAATCGAG
GAGGATTCCGACGAGCCTCTGGAGCGGCGGCTTTCCCTTGTTCCGGACTCGGAACAAGGGGAAGCCATCTTGCCG
CGCATCAGCGTGATCAGCACGGGGCCGACGCTGCAGGCGCGGCGTCGGCAGTCCGTGCTGGCTCTGATGACGCAC
TCTGTGGCGCAAGGGCAGGCCATCCATCGTAAGACGACCGCGTCTACGAGGAAGGTCTCCCTTGCGCCACAGGCT
GCGTTAACAGAGCTGGATATCTACAGCCGCCGTCTCTCCCAGGAGACGGGGCTGGAGATATCCGAAGAGATCGCG
GAGGAGGACTTGAAGGAGTGCTTCTTCGACGATATGGAGTCCATTCCGGCCGTGACCACATGGGCGACATACCTG
CGGTATATTACTGTCCACAAGAGCCTCATCTTTGTCTTGATATGGTGTTTAGTTATTTTCCTTGCCGAAGTGGCG
GCGTCCCTAGTAGTCTTGTGGCTACTAGGGGCGACGCCACTTCAGGACAAGGGAAATAGCACACATAGTAGGAAC
GCGTCGTACGCGGTGATAATCACGTCGACGAGTTCCTACTATGTGTTCTACATCTATGTGGGGGTTGCTGACACG
TTGTTAGCGATGGGCTTCTTCAGGGGGTTACCCCTGGTCCATACGCTGATAACGGTCAGCAAGATCCTCCACCAT
AAGATGTTACACTCGGTGCTTCAAGCACCGATGAGTACGCTCGCAACATTGAAGGCTGGCGGTATCCTGAACCGT
TTCAGCAAAGATATTGCAATACTGGACGACCTGTTGCCCCTGACGATCTTCGACTTCATCCAGTTGTTGTTGATC
GTCATAGGGGCAATAGCGGTCGTCGCAGTGTTGCAGCCCTATATCTTTGTTGCGACGGTTCCGGTTATCGTCGCC
TTCATAATGTTGCGAGCGTACTTCCTCCAGACGTCCCAGCAGCTGAAGCAACTAGAGAGTGAAGGTCGGTCCCCG
ATCTTCACTCATCTAGTTACTTCGCTGAAGGGACTCTGGACGCTGAGGGCATTCGGCAGGCAGCCGTATTTTGAG
ACGCTTTTCCATAAAGCGCTCAATTTACACACGGCTGCCTGGTTCTTGTACCTCAGCACGTTACGGTGGTTCCAG
ATGAGGATAGAGATGATCTTTGTCATCTTCTTTATCGCCGTGACGTTCATCTCCATCCTCACCACCGGTGAGGGG
GAGGGCCGGGTCGGGATCATCCTGACCCTGGCCATGAACATCATGTCAACCCTTCAGTGGGCTGTCGCGAGCTCG
ATAGACGTCGACTCACTGATGCGTTCAGTGAGTCGCGTCTTCAAGTTCATCGACATGCCCACTGAAGGGAAGCCC
ACCAAGAGTACGAAGCCGTACAAGAATGGGCAGCTGTCCAAGGTGATGATTATCGAGAATAGTCACGTCAAGAAG
GACGACATCTGGCCCTCCGGCGGTCAGATGACCGTGAAGGATCTGACCGCCAAGTACACGGAGGGCGGGAACGCC
ATTCTTGAGGCGATTAGCTTCTCGATATCACCTGGACAGCGGGTCGGCCTCCTCGGTCGCACGGGGTCGGGGAAG
AGCACTCTGCTGAGTGCTTTTCTCCGACTCCTTGCGACCGAGGGGGAGATCCAGATAGATGGGGTCTCTTGGGAC
AGTATTACGCTGCAGCAGTGGAGGAAGGCCTTCGGGGTTATACCCCAGAAGGTCTTCATATTCTCCGGGACCTTC
CGGAAGGCTCTTGATCCCTACGAGCAGTGGAGCGATCAAGAGATCTGGAAGGTCGCGGACGAAGTTGGACTGCGC
TCGGTGATAGAGCAGTTCCCCGGGAAGTTGGACTTTGTCTTGGTCGATGGAGGGTGCGTTCTGAGCCACGGCCAT
AAGCAGCTTATGTGCCTGGCTCGGAGCGTACTCTCCAAGGCCAAGATATTGCTGCTGGACGAGCCCAGCGCTCAC
CTCGATCCGGTGACGTACCAGATCATTCGGCGAACGCTGAAGCAAGCGTTCGCCGACTGCACTGTGATCTTGTGC
GAGCACCGGATCGAGGCGATGCTGGAGTGCCAGCAGTTCCTGGTGATCGAAGAGGCTAAGGTCCGCCAGTATGAT
TCGATCCAGAAGTTATTGGCCGAGCGAAGCCTTTTCCGGCAGGCGATCTCTCCAAGCGACCGGGTGAAGCTGTTT
CCTCATCGGGCCTCCTCCAAGTGCAAGTCAAAGCCGCAGATTGCGGCTTTGAAGGAGGAGACCGAGGAGGAAGTG
CAGGACACCCGGTTGTAA
>ATP7B (SEQ ID NO: 15)
ATGCCTGAGCAGGAGAGACAGATCACAGCCAGAGAAGGGGCCAGTCGGAAAATCTTATCTAAGCTTTCTTTGCCT
ACCCGTGCCTGGGAACCAGCAATGAAGAAGAGTTTTGCTTTTGACAATGTTGGCTATGAAGGTGGTCTGGATGGC
CTGGGCCCTTCTTCTCAGGTGGCCACCAGCACAGTCAGGATCTTGGGCATGACTTGCCAGTCATGTGTGAAGTCC
ATTGAGGACAGGATTTCCAATTTGAAAGGCATCATCAGCATGAAGGTTTCCCTGGAACAAGGCAGTGCCACTGTG
AAATATGTGCCATCGGTTGTGTGCCTGCAACAGGTTTGCCATCAAATTGGGGACATGGGCTTCGAGGCCAGCATT
GCAGAAGGAAAGGCAGCCTCCTGGCCCTCAAGGTCCTTGCCTGCCCAGGAGGCTGTGGTCAAGCTCCGGGTGGAG
GGCATGACCTGCCAGTCCTGTGTCAGCTCCATTGAAGGCAAGGTCCGGAAACTGCAAGGAGTAGTGAGAGTCAAA
GTCTCACTCAGCAACCAAGAGGCCGTCATCACTTATCAGCCTTATCTCATTCAGCCCGAAGACCTCAGGGACCAT
GTAAATGACATGGGATTTGAAGCTGCCATCAAGAGCAAAGTGGCTCCCTTAAGCCTGGGACCAATTGATATTGAG
CGGTTACAAAGCACTAACCCAAAGAGACCTTTATCTTCTGCTAACCAGAATTTTAATAATTCTGAGACCTTGGGG
CACCAAGGAAGCCATGTGGTCACCCTCCAACTGAGAATAGATGGAATGCATTGTAAGTCTTGCGTCTTGAATATT
GAAGAAAATATTGGCCAGCTCCTAGGGGTTCAAAGTATTCAAGTGTCCTTGGAGAACAAAACTGCCCAAGTAAAG
TATGACCCTTCTTGTACCAGCCCAGTGGCTCTGCAGAGGGCTATCGAGGCACTTCCACCTGGGAATTTTAAAGTT
TCTCTTCCTGATGGAGCCGAAGGGAGTGGGACAGATCACAGGTCTTCCAGTTCTCATTCCCCTGGCTCCCCACCG
AGAAACCAGGTCCAGGGCACATGCAGTACCACTCTGATTGCCATTGCCGGCATGACCTGTGCATCCTGTGTCCAT
TCCATTGAAGGCATGATCTCCCAACTGGAAGGGGTGCAGCAAATATCGGTGTCTTTGGCCGAAGGGACTGCAACA
GTTCTTTATAATCCCTCTGTAATTAGCCCAGAAGAACTCAGAGCTGCTATAGAAGACATGGGATTTGAGGCTTCA
GTCGTTTCTGAAAGCTGTTCTACTAACCCTCTTGGAAACCACAGTGCTGGGAATTCCATGGTGCAAACTACAGAT
GGTACACCTACATCTGTGCAGGAAGTGGCTCCCCACACTGGGAGGCTCCCTGCAAACCATGCCCCGGACATCTTG
GCAAAGTCCCCACAATCAACCAGAGCAGTGGCACCGCAGAAGTGCTTCTTACAGATCAAAGGCATGACCTGTGCA
TCCTGTGTGTCTAACATAGAAAGGAATCTGCAGAAAGAAGCTGGTGTTCTCTCCGTGTTGGTTGCCTTGATGGCA
GGAAAGGCAGAGATCAAGTATGACCCAGAGGTCATCCAGCCCCTCGAGATAGCTCAGTTCATCCAGGACCTGGGT
TTTGAGGCAGCAGTCATGGAGGACTACGCAGGCTCCGATGGCAACATTGAGCTGACAATCACAGGGATGACCTGC
GCGTCCTGTGTCCACAACATAGAGTCCAAACTCACGAGGACAAATGGCATCACTTATGCCTCCGTTGCCCTTGCC
ACCAGCAAAGCCCTTGTTAAGTTTGACCCGGAAATTATCGGTCCACGGGATATTATCAAAATTATTGAGGAAATT
GGCTTTCATGCTTCCCTGGCCCAGAGAAACCCCAACGCTCATCACTTGGACCACAAGATGGAAATAAAGCAGTGG
AAGAAGTCTTTCCTGTGCAGCCTGGTGTTTGGCATCCCTGTCATGGCCTTAATGATCTATATGCTGATACCCAGC
AACGAGCCCCACCAGTCCATGGTCCTGGACCACAACATCATTCCAGGACTGTCCATTCTAAATCTCATCTTCTTT
ATCTTGTGTACCTTTGTCCAGCTCCTCGGTGGGTGGTACTTCTACGTTCAGGCCTACAAATCTCTGAGACACAGG
TCAGCCAACATGGACGTGCTCATCGTCCTGGCCACAAGCATTGCTTATGTTTATTCTCTGGTCATCCTGGTGGTT
GCTGTGGCTGAGAAGGCGGAGAGGAGCCCTGTGACATTCTTCGACACGCCCCCCATGCTCTTTGTGTTCATTGCC
CTGGGCCGGTGGCTGGAACACTTGGCAAAGAGCAAAACCTCAGAAGCCCTGGCTAAACTCATGTCTCTCCAAGCC
ACAGAAGCCACCGTTGTGACCCTTGGTGAGGACAATTTAATCATCAGGGAGGAGCAAGTCCCCATGGAGCTGGTG
CAGCGGGGCGATATCGTCAAGGTGGTCCCTGGGGGAAAGTTTCCAGTGGATGGGAAAGTCCTGGAAGGCAATACC
ATGGCTGATGAGTCCCTCATCACAGGAGAAGCCATGCCAGTCACTAAGAAACCCGGAAGCACTGTAATTGCGGGG
TCTATAAATGCACATGGCTCTGTGCTCATTAAAGCTACCCACGTGGGCAATGACACCACTTTGGCTCAGATTGTG
AAACTGGTGGAAGAGGCTCAGATGTCAAAGGCACCCATTCAGCAGCTGGCTGACCGGTTTAGTGGATATTTTGTC
CCATTTATCATCATCATGTCAACTTTGACGTTGGTGGTATGGATTGTAATCGGTTTTATCGATTTTGGTGTTGTT
CAGAGATACTTTCCTAACCCCAACAAGCACATCTCCCAGACAGAGGTGATCATCCGGTTTGCTTTCCAGACGTCC
ATCACGGTGCTGTGCATTGCCTGCCCCTGCTCCCTGGGGCTGGCCACGCCCACGGCTGTCATGGTGGGCACCGGG
GTGGCCGCGCAGAACGGCATCCTCATCAAGGGAGGCAAGCCCCTGGAGATGGCGCACAAGATAAAGACTGTGATG
TTTGACAAGACTGGCACCATTACCCATGGCGTCCCCAGGGTCATGCGGGTGCTCCTGCTGGGGGATGTGGCCACA
CTGCCCCTCAGGAAGGTTCTGGCTGTGGTGGGGACTGCGGAGGCCAGCAGTGAACACCCCTTGGGCGTGGCAGTC
ACCAAATACTGTAAAGAGGAACTTGGAACAGAGACCTTGGGATACTGCACGGACTTCCAGGCAGTGCCAGGCTGT
GGAATTGGGTGCAAAGTCAGCAACGTGGAAGGCATCCTGGCCCACAGTGAGCGCCCTTTGAGTGCACCGGCCAGT
CACCTGAATGAGGCTGGCAGCCTTCCCGCAGAAAAAGATGCAGTCCCCCAGACCTTCTCTGTGCTGATTGGAAAC
CGTGAGTGGCTGAGGCGCAACGGTTTAACCATTTCTAGCGATGTCAGTGACGCTATGACAGACCACGAGATGAAA
GGACAGACAGCCATCCTGGTGGCTATTGACGGTGTGCTCTGTGGGATGATCGCAATCGCAGACGCTGTCAAGCAG
GAGGCTGCCCTGGCTGTGCACACGCTGCAGAGCATGGGTGTGGACGTGGTTCTGATCACGGGGGACAACCGGAAG
ACAGCCAGAGCTATTGCCACCCAGGTTGGCATCAACAAAGTCTTTGCAGAGGTGCTGCCTTCGCACAAGGTGGCC
AAGGTCCAGGAGCTCCAGAATAAAGGGAAGAAAGTCGCCATGGTGGGGGATGGGGTCAATGACTCCCCGGCCTTG
GCCCAGGCAGACATGGGTGTGGCCATTGGCACCGGCACGGATGTGGCCATCGAGGCAGCCGACGTCGTCCTTATC
AGAAATGATTTGCTGGATGTGGTGGCTAGCATTCACCTTTCCAAGAGGACTGTCCGAAGGATACGCATCAACCTG
GTCCTGGCACTGATTTATAACCTGGTTGGGATACCCATTGCAGCAGGTGTCTTCATGCCCATCGGCATTGTGCTG
CAGCCCTGGATGGGCTCAGCGGCCATGGCAGCCTCCTCTGTGTCTGTGGTGCTCTCATCCCTGCAGCTCAAGTGC
TATAAGAAGCCTGACCTGGAGAGGTATGAGGCACAGGCGCATGGCCACATGAAGCCCCTGACGGCATCCCAGGTC
AGTGTGCACATAGGCATGGATGACAGGTGGCGGGACTCCCCCAGGGCCACACCATGGGACCAGGTCAGCTATGTC
AGCCAGGTGTCGCTGTCCTCCCTGACGTCCGACAAGCCATCTCGGCACAGCGCTGCAGCAGACGATGATGGGGAC
AAGTGGTCTCTGCTCCTGAATGGCAGGGATGAGGAGCAGTACATCTGA
>ATP7A (SEQ ID NO: 16)
ATGGATCCAAGTATGGGTGTGAATTCTGTTACCATTTCTGTTGAGGGTATGACTTGCAATTCCTGTGTTTGGACC
ATTGAGCAGCAGATTGGAAAAGTGAATGGTGTGCATCACATTAAGGTATCACTGGAAGAAAAAAATGCAACTATT
ATTTATGACCCTAAACTACAGACTCCAAAGACCCTACAGGAAGCTATTGATGACATGGGCTTTGATGCTGTTATC
CATAATCCTGACCCTCTCCCTGTTTTAACTGACACCTTGTTTCTGACTGTTACGGCGTCACTGACTTTGCCATGG
GACCATATCCAAAGCACATTGCTGAAGACCAAGGGTGTGACAGACATTAAAATTTACCCTCAGAAAAGAACTGTA
GCAGTGACAATAATCCCTTCTATAGTGAATGCCAATCAGATAAAAGAGCTGGTTCCAGAACTCAGTTTAGATACT
GGGACACTGGAGAAAAAGTCAGGAGCTTGTGAAGATCATAGTATGGCTCAAGCTGGTGAAGTCGTGCTGAAGATG
AAAGTGGAAGGGATGACCTGCCATTCATGTACTAGCACTATTGAAGGAAAAATTGGGAAACTGCAAGGTGTTCAG
CGAATTAAAGTCTCCCTGGACAATCAAGAAGCTACTATTGTTTATCAACCTCATCTTATCTCAGTAGAGGAAATG
AAAAAGCAGATTGAAGCTATGGGCTTTCCAGCATTTGTCAAAAAGCAGCCCAAGTACCTCAAATTGGGAGCTATT
GATGTAGAACGTCTAAAGAACACACCAGTTAAATCCTCAGAAGGGTCACAGCAAAGGAGTCCATCATATACCAAT
GATTCAACAGCCACTTTCATCATTGATGGCATGCATTGTAAATCATGTGTGTCAAATATTGAAAGTACTTTATCT
GCACTCCAATATGTAAGCAGCATAGTAGTTTCTTTAGAGAATAGGTCTGCCATTGTGAAGTATAATGCAAGCTCA
GTCACTCCAGAATCCCTGAGAAAAGCAATAGAGGCTGTATCACCGGGGCTATATAGAGTTAGTATCACAAGTGAA
GTTGAGAGTACCTCAAACTCTCCCTCCAGCTCATCTCTTCAGAAGATTCCTTTGAATGTAGTTAGCCAGCCTCTG
ACACAAGAAACTGTGATAAACATTGATGGCATGACTTGTAATTCCTGTGTGCAGTCTATTGAGGGTGTCATATCA
AAAAAGCCAGGTGTAAAATCCATACGAGTCTCCCTTGCAAATAGCAATGGGACTGTTGAGTATGATCCTCTACTA
ACCTCTCCAGAAACGTTGAGAGGAGCAATAGAAGACATGGGATTTGATGCTACCTTGTCAGACACGAATGAGCCG
TTGGTAGTAATAGCTCAGCCTTCATCGGAAATGCCGCTTTTGACTTCAACTAATGAATTTTATACTAAAGGGATG
ACACCAGTTCAAGACAAGGAGGAAGGAAAGAATTCATCTAAGTGTTACATACAGGTCACTGGCATGACTTGCGCT
TCCTGTGTAGCAAACATTGAACGGAATTTAAGGCGGGAAGAAGGAATATATTCTATACTTGTGGCCCTGATGGCT
GGCAAGGCAGAAGTAAGGTATAATCCTGCTGTTATACAACCCCCAATGATAGCAGAGTTCATCCGAGAACTTGGA
TTTGGAGCCACTGTGATAGAAAATGCTGATGAAGGAGATGGTGTTTTGGAACTTGTTGTGAGGGGAATGACGTGT
GCCTCCTGCGTACATAAAATAGAGTCTAGTCTCACAAAACACAGAGGGATCCTATACTGCTCCGTGGCCCTGGCA
ACCAACAAAGCACATATTAAATATGACCCAGAAATTATTGGTCCTAGAGATATTATCCATACAATTGAAAGCTTA
GGTTTTGAAGCTTCTTTGGTCAAGAAGGATCGGTCAGCAAGTCACTTAGATCATAAACGAGAAATAAGACAATGG
AGACGGTCTTTTCTTGTGAGTCTGTTTTTCTGTATTCCTGTAATGGGGCTGATGATATATATGATGGTTATGGAC
CACCACTTTGCAACTCTTCACCATAATCAAAACATGAGTAAAGAAGAAATGATCAACCTTCATTCTTCTATGTTC
CTGGAGCGCCAGATTCTTCCAGGATTGTCTGTTATGAATTTGCTGTCCTTTTTATTGTGTGTACCTGTACAGTTT
TTCGGAGGCTGGTACTTCTACATTCAGGCTTATAAAGCACTGAAGCATAAGACAGCAAATATGGACGTACTGATT
GTGCTGGCAACCACCATTGCATTTGCCTACTCTTTGATTATTCTTCTAGTTGCAATGTATGAGAGAGCCAAAGTG
AACCCTATTACTTTCTTTGACACACCCCCTATGCTGTTTGTGTTTATTGCACTAGGCCGATGGCTGGAACATATA
GCAAAGGGCAAAACATCAGAGGCTCTTGCAAAGTTAATTTCACTACAAGCTACAGAAGCAACTATTGTAACTCTT
GATTCTGATAATATCCTCCTCAGTGAAGAACAAGTGGATGTGGAACTTGTACAACGTGGAGATATCATTAAAGTA
GTTCCAGGAGGCAAATTTCCAGTGGATGGTCGTGTTATTGAAGGACATTCTATGGTAGATGAGTCCCTCATCACA
GGGGAGGCAATGCCTGTGGCTAAGAAACCTGGCAGCACAGTGATTGCTGGTTCCATTAACCAGAACGGGTCACTG
CTTATCTGCGCAACACATGTTGGAGCAGACACAACCCTTTCTCAAATTGTCAAACTTGTGGAAGAGGCACAAACA
TCAAAGGCTCCTATCCAGCAGTTTGCAGACAAACTCAGTGGCTATTTTGTTCCTTTTATTGTTTTTGTTTCCATT
GCCACCCTCTTGGTATGGATTGTAATTGGATTTCTGAATTTTGAAATTGTGGAAACCTACTTTCCTGGCTACAAT
AGAAGTATCTCCCGAACAGAAACGATAATACGATTTGCTTTCCAAGCCTCTATCACAGTTCTGTGTATTGCATGT
CCCTGTTCACTGGGACTGGCCACTCCAACTGCTGTGATGGTGGGTACAGGAGTAGGTGCTCAAAATGGCATACTA
ATAAAAGGTGGAGAGCCATTGGAGATGGCTCATAAGGTAAAGGTAGTGGTATTTGATAAGACTGGAACCATTACT
CACGGAACCCCAGTGGTGAATCAAGTAAAGGTTCTAACTGAAAGTAACAGAATATCACACCATAAAATCTTGGCC
ATTGTGGGAACTGCTGAAAGTAACAGTGAACACCCTCTAGGAACAGCCATAACCAAATATTGCAAACAGGAGCTG
GACACTGAAACCTTGGGTACCTGCATAGATTTCCAGGTTGTGCCAGGCTGTGGTATTAGCTGTAAAGTCACCAAT
ATTGAAGGCTTGCTACATAAGAATAACTGGAATATAGAGGACAATAATATTAAAAATGCATCCCTGGTTCAAATT
GATGCCAGTAATGAACAGTCATCAACTTCGTCTTCCATGATTATTGATGCCCAGATCTCAAATGCTCTTAATGCT
CAGCAGTATAAAGTCCTCATTGGTAACCGGGAGTGGATGATTAGAAATGGTCTTGTCATTAATAACGATGTAAAT
GATTTCATGACTGAACATGAGAGAAAAGGTCGGACTGCTGTATTAGTAGCAGTTGATGATGAGCTGTGTGGCTTG
ATAGCCATTGCAGACACAGTGAAGCCTGAAGCAGAACTGGCTATCCATATTCTGAAATCTATGGGCTTAGAAGTA
GTTCTGATGACTGGAGACAACAGTAAAACAGCTAGATCTATTGCTTCTCAGGTTGGCATTACTAAGGTGTTTGCT
GAAGTTCTACCTTCTCACAAGGTTGCTAAAGTGAAGCAACTTCAAGAGGAGGGGAAACGGGTAGCAATGGTGGGA
GATGGAATCAATGACTCCCCAGCTCTGGCAATGGCTAATGTGGGAATTGCTATTGGCACAGGCACAGATGTAGCC
ATTGAAGCAGCTGATGTGGTTTTGATAAGGAATGATCTTCTGGATGTAGTGGCAAGTATTGACTTATCAAGAAAG
ACAGTCAAGAGGATTCGGATAAATTTTGTCTTTGCTCTAATTTATAATCTGGTTGGAATTCCCATAGCTGCTGGA
GTTTTTATGCCCATTGGTTTGGTTTTGCAGCCCTGGATGGGATCTGCAGCAATGGCTGCTTCATCTGTTTCTGTA
GTACTTTCTTCTCTCTTCCTTAAACTTTACAGGAAACCAACTTACGAGAGTTATGAACTGCCTGCCCGGAGCCAG
ATAGGACAGAAGAGTCCTTCAGAAATCAGCGTTCATGTTGGAATAGATGATACCTCAAGGAATTCTCCTAAACTG
GGTTTGCTGGACCGGATTGTTAATTATAGCAGAGCCTCTATAAACTCACTACTGTCTGATAAACGCTCCCTAAAC
AGTGTTGTTACCAGTGAACCTGACAAGCACTCACTCCTGGTGGGAGACTTCAGGGAAGATGATGACACTGCATTA
TAA
>AGL (SEQ ID NO: 17)
ATGGGACACAGTAAACAGATTCGAATTTTACTTCTGAACGAAATGGAGAAACTGGAAAAGACCCTCTTCAGACTT
GAACAAGGGTATGAGCTACAGTTCCGATTAGGCCCAACTTTACAGGGAAAAGCAGTTACCGTGTATACAAATTAC
CCATTTCCTGGAGAAACATTTAATAGAGAAAAATTCCGTTCTCTGGATTGGGAAAATCCAACAGAAAGAGAAGAT
GATTCTGATAAATACTGTAAACTTAATCTGCAACAATCTGGTTCATTTCAGTATTATTTCCTTCAAGGAAATGAG
AAAAGTGGTGGAGGTTACATAGTTGTGGACCCCATTTTACGTGTTGGTGCTGATAATCATGTGCTACCCTTGGAC
TGTGTTACTCTTCAGACATTTTTAGCTAAGTGTTTGGGACCTTTTGATGAATGGGAAAGCAGACTTAGGGTTGCA
AAAGAATCAGGCTACAACATGATTCATTTTACCCCATTGCAGACTCTTGGACTATCTAGGTCATGCTACTCCCTT
GCCAATCAGTTAGAATTAAATCCTGACTTTTCAAGACCTAATAGAAAGTATACCTGGAATGATGTTGGACAGCTA
GTGGAAAAATTAAAAAAGGAATGGAATGTTATTTGTATTACTGATGTTGTCTACAATCATACTGCTGCTAATAGT
AAATGGATCCAGGAACATCCAGAATGTGCCTATAATCTTGTGAATTCTCCACACTTAAAACCTGCCTGGGTCTTA
GACAGAGCACTTTGGCGTTTCTCCTGTGATGTTGCAGAAGGGAAATACAAAGAAAAGGGAATACCTGCTTTGATT
GAAAATGATCACCATATGAATTCCATCCGAAAAATAATTTGGGAGGATATTTTTCCAAAGCTTAAACTCTGGGAA
TTTTTCCAAGTAGATGTCAACAAAGCGGTTGAGCAATTTAGAAGACTTCTTACACAAGAAAATAGGCGAGTAACC
AAGTCTGATCCAAACCAACACCTTACGATTATTCAAGATCCTGAATACAGACGGTTTGGCTGTACTGTAGATATG
AACATTGCACTAACGACTTTCATACCACATGACAAGGGGCCAGCAGCAATTGAAGAATGCTGTAATTGGTTTCAT
AAAAGAATGGAGGAATTAAATTCAGAGAAGCATCGACTCATTAACTATCATCAGGAACAGGCAGTTAATTGCCTT
TTGGGAAATGTGTTTTATGAACGACTGGCTGGCCATGGTCCAAAACTAGGACCTGTCACTAGAAAGCATCCTTTA
GTTACCAGGTATTTTACTTTCCCATTTGAAGAGATAGACTTCTCCATGGAAGAATCTATGATTCATCTGCCAAAT
AAAGCTTGTTTTCTGATGGCACACAATGGATGGGTAATGGGAGATGATCCTCTTCGAAACTTTGCTGAACCGGGT
TCAGAAGTTTACCTAAGGAGAGAACTTATTTGCTGGGGAGACAGTGTTAAATTACGCTATGGGAATAAACCAGAG
GACTGTCCTTATCTCTGGGCACACATGAAAAAATACACTGAAATAACTGCAACTTATTTCCAGGGAGTACGTCTT
GATAACTGCCACTCAACACCTCTTCACGTAGCTGAGTACATGTTGGATGCTGCTAGGAATTTGCAACCCAATTTA
TATGTAGTAGCTGAACTGTTCACAGGAAGTGAAGATCTGGACAATGTCTTTGTTACTAGACTGGGCATTAGTTCC
TTAATAAGAGAGGCAATGAGTGCATATAATAGTCATGAAGAGGGCAGATTAGTTTACCGATATGGAGGAGAACCT
GTTGGATCCTTTGTTCAGCCCTGTTTGAGGCCTTTAATGCCAGCTATTGCACATGCCCTGTTTATGGATATTACG
CATGATAATGAGTGTCCTATTGTGCATAGATCAGCGTATGATGCTCTTCCAAGTACTACAATTGTTTCTATGGCA
TGTTGTGCTAGTGGAAGTACAAGAGGCTATGATGAATTAGTGCCTCATCAGATTTCAGTGGTTTCTGAAGAACGG
TTTTACACTAAGTGGAATCCTGAAGCATTGCCTTCAAACACAGGTGAAGTTAATTTCCAAAGCGGCATTATTGCA
GCCAGGTGTGCTATCAGTAAACTTCATCAGGAGCTTGGAGCCAAGGGTTTTATTCAGGTGTATGTGGATCAAGTT
GATGAAGACATAGTGGCAGTAACAAGACACTCACCTAGCATCCATCAGTCTGTTGTGGCTGTATCTAGAACTGCT
TTCAGGAATCCCAAGACTTCATTTTACAGCAAGGAAGTGCCTCAAATGTGCATCCCTGGCAAAATTGAAGAAGTA
GTTCTTGAAGCTAGAACTATTGAGAGAAACACGAAACCTTATAGGAAGGATGAGAATTCAATCAATGGAACACCA
GATATCACAGTAGAAATTAGAGAACATATTCAGCTTAATGAAAGTAAAATTGTTAAACAAGCTGGAGTTGCCACA
AAAGGGCCCAATGAATATATTCAAGAAATAGAATTTGAAAACTTGTCTCCAGGAAGTGTTATTATATTCAGAGTT
AGTCTTGATCCACATGCACAAGTCGCTGTTGGAATTCTTCGAAATCATCTGACACAATTCAGTCCTCACTTTAAA
TCTGGCAGCCTAGCTGTTGACAATGCAGATCCTATATTAAAAATTCCTTTTGCTTCTCTTGCCTCCAGATTAACT
TTGGCTGAGCTAAATCAGATCCTTTACCGATGTGAATCAGAAGAAAAGGAAGATGGTGGAGGGTGCTATGACATA
CCAAACTGGTCAGCCCTTAAATATGCAGGTCTTCAAGGTTTAATGTCTGTATTGGCAGAAATAAGACCAAAGAAT
GACTTGGGGCATCCTTTTTGTAATAATTTGAGATCTGGAGATTGGATGATTGACTATGTCAGTAACCGGCTTATT
TCACGATCAGGAACTATTGCTGAAGTTGGTAAATGGTTGCAGGCTATGTTCTTCTACCTGAAGCAGATCCCACGT
TACCTTATCCCATGTTACTTTGATGCTATATTAATTGGTGCATATACCACTCTTCTGGATACAGCATGGAAGCAG
ATGTCAAGCTTTGTTCAGAATGGTTCAACCTTTGTGAAACACCTTTCATTGGGTTCAGTTCAACTGTGTGGAGTA
GGAAAATTCCCTTCCCTGCCAATTCTTTCACCTGCCCTAATGGATGTACCTTATAGGTTAAATGAGATCACAAAA
GAAAAGGAGCAATGTTGTGTTTCTCTAGCTGCAGGCTTACCTCATTTTTCTTCTGGTATTTTCCGCTGCTGGGGA
AGGGATACTTTTATTGCACTTAGAGGTATACTGCTGATTACTGGACGCTATGTAGAAGCCAGGAATATTATTTTA
GCATTTGCGGGTACCCTGAGGCATGGTCTCATTCCTAATCTACTGGGTGAAGGAATTTATGCCAGATACAATTGT
CGGGATGCTGTGTGGTGGTGGCTGCAGTGTATCCAGGATTACTGTAAAATGGTTCCAAATGGTCTAGACATTCTC
AAGTGCCCAGTTTCCAGAATGTATCCTACAGATGATTCTGCTCCTTTGCCTGCTGGCACACTGGATCAGCCATTG
TTTGAAGTCATACAGGAAGCAATGCAAAAACACATGCAGGGCATACAGTTCCGAGAAAGGAATGCTGGTCCCCAG
ATAGATCGAAACATGAAGGACGAAGGTTTTAATATAACTGCAGGAGTTGATGAAGAAACAGGATTTGTTTATGGA
GGAAATCGTTTCAATTGTGGCACATGGATGGATAAAATGGGAGAAAGTGACAGAGCTAGAAACAGAGGAATCCCA
GCCACACCAAGAGATGGGTCTGCTGTGGAAATTGTGGGCCTGAGTAAATCTGCTGTTCGCTGGTTGCTGGAATTA
TCCAAAAAAAATATTTTCCCTTATCATGAAGTCACAGTAAAAAGACATGGAAAGGCTATAAAGGTCTCATATGAT
GAGTGGAACAGAAAAATACAAGACAACTTTGAAAAGCTATTTCATGTTTCCGAAGACCCTTCAGATTTAAATGAA
AAGCATCCAAATCTGGTTCACAAACGTGGCATATACAAAGATAGTTATGGAGCTTCAAGTCCTTGGTGTGACTAT
CAGCTCAGGCCTAATTTTACCATAGCAATGGTTGTGGCCCCTGAGCTCTTTACTACAGAAAAAGCATGGAAAGCT
TTGGAGATTGCAGAAAAAAAATTGCTTGGTCCCCTTGGCATGAAAACTTTAGATCCAGATGATATGGTTTACTGT
GGAATTTATGACAATGCATTAGACAATGACAACTACAATCTTGCTAAAGGTTTCAATTATCACCAAGGACCTGAG
TGGCTGTGGCCTATTGGGTATTTTCTTCGTGCAAAATTATATTTTTCCAGATTGATGGGCCCGGAGACTACTGCA
AAGACTATAGTTTTGGTTAAAAATGTTCTTTCCCGACATTATGTTCATCTTGAGAGATCCCCTTGGAAAGGACTT
CCAGAACTGACCAATGAGAATGCCCAGTACTGTCCTTTCAGCTGTGAAACACAAGCCTGGTCAATTGCTACTATT
CTTGAGACACTTTATGATTTATAG
>DMD_mingene (SEQ ID NO: 18)
ATGCTTTGGTGGGAAGAAGTAGAGGACTGTTATGAAAGAGAAGATGTTCAAAAGAAAACATTCACAAAATGGGTA
AATGCACAATTTTCTAAGTTTGGGAAGCAGCATATTGAGAACCTCTTCAGTGACCTACAGGATGGGAGGCGCCTC
CTAGACCTCCTCGAAGGCCTGACAGGGCAAAAACTGCCAAAAGAAAAAGGATCCACAAGAGTTCATGCCCTGAAC
AATGTCAACAAGGCACTGCGGGTTTTGCAGAACAATAATGTTGATTTAGTGAATATTGGAAGTACTGACATCGTA
GATGGAAATCATAAACTGACTCTTGGTTTGATTTGGAATATAATCCTCCACTGGCAGGTCAAAAATGTAATGAAA
AATATCATGGCTGGATTGCAACAAACCAACAGTGAAAAGATTCTCCTGAGCTGGGTCCGACAATCAACTCGTAAT
TATCCACAGGTTAATGTAATCAACTTCACCACCAGCTGGTCTGATGGCCTGGCTTTGAATGCTCTCATCCATAGT
CATAGGCCAGACCTATTTGACTGGAATAGTGTGGTTTGGCAGCAGTCAGCCACACAACGACTGGAACATGCATTC
AACATCGCCAGATATCAATTAGGCATAGAGAAAGTACTCGATCCTGAAGATGTTGATACCACCTATCCAGATAAG
AAGTCCATCTTAATGTACATCACATCACTCTTCCAAGTTTTGCCTCAACAAGTGAGCATTGAAGCCATCCAGGAA
GTGGAAATGTTGCCAAGGCCACCTAAAGTGACTAAAGAAGAACATTTTCAGTACATCATCAAATGCACTATTCTC
AACAGATCACGGTCAGTCTAGCACAGGGATATGAGAGAACTTCTTCCCCTAAGCCTCGATTCAAGAGCTATGCCT
ACACACAGGCTGCTTATGTCACCACCTCTGACCCTACACGGAGCCCATTTCCTTCACAGCATTTGGAAGCTCCTG
AAGACAAGTCATTTGGCAGTTCATTGATGGAGAGTGAAGTAAACCTGGACCGTTATCAAACAGCTTTAGAAGAAG
TATTATCGTGGCTTCTTTCTGCTGAGGACACATTGCAAGCACAAGGAGAGATTTCTAATGATGTGGAAGTGGTGA
AAGACCAGTTTCATACTCATGAGGGGTACATGATGGATTTGACAGCCCATCAGGGCCGGGTTGGTAATATTCTAC
AATTGGGAAGTAAGCTGATTGGAACAGGAAAATTATCAGAAGATGAAGAAACTGAAGTACAAGAGCAGATGAATC
TCCTAAATTCAAGATGGGAATGCCTCAGGGTAGCTAGCATGGAAAAACAAAGCAATTTACATAGAGTTTTAATGG
ATCTCCAGAATCAGAAACTGAAAGAGTTGAATGACTGGCTAACAAAAACAGAAGAAAGAACAAGGAAAATGGAGG
AAGAGCCTCTTGGACCTGATCTTGAAGACCTAAAACGCCAAGTACAACAACATAAGGTGCTTCAAGAAGATCTAG
AACAAGAACAAGTCAGGGTCAATTCTCTCACTCACATGGTGGTGGTAGTTGATGAATCTAGTGGAGATCACGCAA
CTGCTGCTTTGGAAGAACAAGTTTAAGGTATTGGGAGATCGATGGGCAAACATCTGTAGATGGACAGAAGACCGC
TGGGTTCTTTTACAAGACATCCTTCTCAAATGGCAACGTCTTACTGAAGAACAGTGCCTTTTTAGTGCATGGCTT
TCAGAAAAAGAAGATGCAGTGAACAAGATTCACACAACTGGCTTTAAAGATCAAAATGAAATGTTATCAAGTCTT
CAAAAACTGGCCGTTTTAAAAGCGGATCTAGAAAAGAAAAAGCAATCCATGGGCAAACTGTATTCACTCAAACAA
GATCTTCTTTCAACACTGAAGAATAAGTCAGTGACCCAGAAGACGGAAGCATGGCTGGATAACTTTGCCCGGTGT
TGGGATAATTTAGTCCAAAAACTTGAAAAGAGTACAGCACAGGAAACTGAAATAGCAGTTCAAGCTAAACAACCG
GATGTGGAAGAGATTTTGTCTAAAGGGCAGCATTTGTACAAGGAAAAACCAGCCACTCAGCCAGTGAAGAGGAAG
TTAGAAGATCTGAGCTCTGAGTGGAAGGCGGTAAACCGTTTACTTCAAGAGCTGAGGGCAAAGCAGCCTGACCTA
GCTCCTGGACTGACCACTATTGGAGCCTCTCCTACTCAGACTGTTACTCTGGTGACACAACCTGTGGTTAGTAAG
GAAACTGCCATCTCCAAACTAGAAATGCCATCTTCCTTGATGTTGGAGGTACCTGCTCTGGCAGATTTCAACGGG
GCTTGGACAGAACTTACCGAGTGGCTTTCTCTGCTTGATCAAGTTATAAAATCACAGAGGGTGATGGTGGGTGAC
CTTGAGGATATCAACGAGATGATCATCAAGCAGAAGGCAACAATGCAGGATTTGGAACAGAGGCGTCCCCAGTTG
GAAGAACTCATTACCGCTGCCCAAAATTTGAAAAACAAGACCAGCAATCAAGAGGCTAGAACAATCATTACGGAT
CGAATTGAAAGAATTCAGAATCAGTGGGATGAAGTACAAGAACACCTTCAGAACCGGAGGCAACAGTTGAATGAA
ATGTTAAAGGATTCAACACAATGGCTGGAAGCTAAGGAAGAAGCTGAGCAGGTCTTAGGACAGGGCAGAGCCAAG
CTTGAGTCATGGAAGGAGGGTCCCTATACAGTAGATGCAATCCAAAAGAAAATCACAGAAACCAAGCAGTTGGCC
AAAGACCTCCGCCAGTGGCAGACAAATGTAGATGTGGCAAATGACTTGGCCCTGAAACTTCTCCGGGATTATTCT
GCAGATGATACCAGAAAAGTCCACATGATAACAGAGAATATCAATGCCTCTTGGAGAAGCATTCATAAAAGGGTG
AGTGAGCGAGAGGCTGCTTTGGAAGAAACTCATAGATTACTGCAACAGTTCCCCCTGGACCTGGAAAAGTTTCTT
GCCTGGCTTACAGAAGCTGAAACAACTGCCAATGTCCTACAGGATGCTACCCGTAAGGAAAGGCTCCTAGAAGAC
TCCAAGGGAGTAAAAGAGCTGATGAAACAATGGCAAGACCTCCAAGGTGAAATTGAAGCTCACACAGATGTTTAT
CACAACCTGGATGAAAACAGCCAAAAAATCCTGAGATCCCTGGAAGGTTCCGATGATGCAGTCCTGTTACAAAGA
CGTTTGGATAACATGAACTTCAAGTGGAGTGAACTTCGGAAAAAGTCTCTCAACATTAGGTCCCATTTGGAAGCC
AGTTCTGACCAGTGGAAGCGTCTGCACCTTTCTCTGCAGGAACTTCTGGTGTGGCTACAGCTGAAAGATGATGAA
TTAAGCCGGCAGGCACCTATTGGAGGCGACTTTCCAGCAGTTCAGAAGCAGAACGATGTACATAGGGCCTTCAAG
AGGGAATTGAAAACTAAAGAACCTGTAATCATGAGTACTCTTGAGACTGTACGAATATTTCTGACAGAGCAGCCT
TTGGAAGGACTAGAGAAACTCTACCAGGAGCCCAGAGAGCTGCCTCCTGAGGAGAGAGCCCAGAATGTCACTCGG
CTTCTACGAAAGCAGGCTGAGGAGGTCAATACTGAGTGGGAAAAATTGAACCTGCACTCCGCTGACTGGCAGAGA
AAAATAGATGAGACCCTTGAAAGACTCCAGGAACTTCAAGAGGCCACGGATGAGCTGGACCTCAAGCTGCGCCAA
GCTGAGGTGATCAAGGGATCCTGGCAGCCCGTGGGCGATCTCCTCATTGACTCTCTCCAAGATCACCTCGAGAAA
GTCAAGGCACTTCGAGGAGAAATTGCGCCTCTGAAAGAGAACGTGAGCCACGTCAATGACCTTGCTCGCCAGCTT
ACCACTTTGGGCATTCAGCTCTCACCGTATAACCTCAGCACTCTGGAAGACCTGAACACCAGATGGAAGCTTCTG
CAGGTGGCCGTCGAGGACCGAGTCAGGCAGCTGCATGAAGCCCACAGGGACTTTGGTCCAGCATCTCAGCACTTT
CTTTCCACGTCTGTCCAGGGTCGGTGGGAGAGAGCCATCTCGCCAAACAAAGTGCCCTACTATATCAACCACGAG
ACTCAAACAACTTGCTGGGACCATCCCAAAATGACAGAGCTCTACCAGTCTTTAGCTGACCTGAATAATGTCAGA
TTCTCAGCTTATAGGACTGCCATGAAACTCCGAAGACTGCAGAAGGCCCTTTGCTTGGATCTCTTGAGCCTGTCA
GCTGCATGTGATGCCTTGGACCAGCACAACCTCAAGCAAAATGACCAGCCCATGGATATCCTGCAGATTATTAAT
TGTTTGACCACTATTTATGACCGCCTGGAGCAAGAGCACAACAATTTGGTCAACGTCCCTCTCTGCGTGGATATG
TGTCTGAACTGGCTGCTGAATGTTTATGATACGGGACGAACAGGGAGGATCCGTGTCCTGTCTTTTAAAACTGGC
ATCATTTCCCTGTGTAAAGCACATTTGGAAGACAAGTACAGATACCTTTTCAAGCAAGTGGCAAGTTCAACAGGA
TTTTGTGACCAGCGCAGGCTGGGCCTCCTTCTGCATGATTCTATCCAAATTCCAAGACAGTTGGGTGAAGTTGCA
TCCTTTGGGGGCAGTAACATTGAGCCAAGTGTCCGGAGCTGCTTCCAATTTGCTAATAATAAGCCAGAGATCGAA
GCGGCCCTCTTCCTAGACTGGATGAGACTGGAACCCCAGTCCATGGTGTGGCTGCCCGTCCTGCACAGAGTGGCT
GCTGCAGAAACTGCCAAGCATCAGGCCAAATGTAACATCTGCAAAGAGTGTCCAATCATTGGATTCAGGTACAGG
AGTCTAAAGCACTTTAATTATGACATCTGCCAAAGCTGCTTTTTTTCTGGTCGAGTTGCAAAAGGCCATAAAATG
CACTATCCCATGGTGGAATATTGCACTCCGACTACATCAGGAGAAGATGTTCGAGACTTTGCCAAGGTACTAAAA
AACAAATTTCGAACCAAAAGGTATTTTGCGAAGCATCCCCGAATGGGCTACCTGCCAGTGCAGACTGTCTTAGAG
GGGGACAACATGGAAACTCCCGTTACTCTGATCAACTTCTGGCCAGTAGATTCTGCGCCTGCCTCGTCCCCTCAG
CTTTCACACGATGATACTCATTCACGCATTGAACATTATGCTAGCAGGCTAGCAGAAATGGAAAACAGCAATGGA
TCTTATCTAAATGATAGCATCTCTCCTAATGAGAGCATAGATGATGAACATTTGTTAATCCAGCATTACTGCCAA
AGTTTGAACCAGGACTCCCCCCTGAGCCAGCCTCGTAGTCCTGCCCAGATCTTGATTTCCTTAGAGAGTGAGGAA
AGAGGGGAGCTAGAGAGAATCCTAGCAGATCTTGAGGAAGAAAACAGGAATCTGCAAGCAGAATATGACCGTCTA
AAGCAGCAGCACGAACATAAAGGCCTGTCCCCACTGCCGTCCCCTCCTGAAATGATGCCCACCTCTCCCCAGAGT
CCCCGGGATGCTGAGCTCATTGCTGAGGCCAAGCTACTGCGTCAACACAAAGGCCGCCTGGAAGCCAGGATGCAA
ATCCTGGAAGACCACAATAAACAGCTGGAGTCACAGTTACACAGGCTAAGGCAGGTGCTGGAGCAACCCCAGGCA
GAGGCCAAAGTGAATGGCACAACGGTGTCCTCTCCTTCTACCTCTCTACAGAGGTCCGACAGCAGTCAGCCTATG
CTGCTCCGAGTGGTTGGCAGTCAAACTTCGGACTCCATGGGTGAGGAAGATCTTCTCAGTCGTCCCCAGGACACA
AGCACAGGGTTAGAGGAGGTGATGGAGCAACTCAACAACTCCTTTCCCTAGTTCAAGAGGAAGAAATACCCCTGG
AAAGCCAATGAGAGAGGACACAATGTAG
>CPS1_NM_001145134.2 (SEQ ID NO: 19)
AGACCTTTGAGCAACCTTCACCGCACAGAAACCCAGCCGCGCCCTGCAATTCCCACCGCGGAAGGTGCCGACCAA
CCCCCAGGATGGCGGAAGCTCACCAGGCCGTGGCCTTCCAGTTCACGGTGACCCCAGACGGGGTCGACTTCCGGC
TCAGTCGGGAGGCCCTGAAACACGTCTACCTGTCTGGGATCAACTCCTGGAAGAAACGCCTGATCCGCATCAAGA
ATGGCATCCTCAGGGGCGTGTACCCTGGCAGCCCCACCAGCTGGCTGGTCGTCATCATGGCAACAGTGGGTTCCT
CCTTCTGCAACGTGGACATCTCCTTGGGGCTGGTCAGTTGCATCCAGAGATGCCTCCCTCAGGGGTGTGGCCCCT
ACCAGACCCCGCAGACCCGGGCACTTCTCAGCATGGCCATCTTCTCCACGGGCGTCTGGGTGACGGGCATCTTCT
TCTTCCGCCAAACCCTGAAGCTGCTTCTCTGCTACCATGGGTGGATGTTTGAGATGCATGGCAAGACCAGCAACT
TGACCAGGATCTGGGCTTACCTAGAGTCTGTGCGCCCCTTGTTGGATGATGAGGAATATTACCGCATGGAGTTGC
TGGCCAAAGAATTCCAGGACAAGACTGCCCCCAGGCTGCAGAAATACCTGGTGCTCAAGTCATGGTGGGCAAGTA
ACTATGTGAGTGACTGGTGGGAAGAGTACATCTACCTTCGAGGCAGGAGCCCTCTCATGGTGAACAGCAACTATT
ATGTCATGGACCTTGTGCTC
ATCAAGAATACAGACGTGCAGGCAGCCCGCCTGGGAAACATCATCCACGCCATGATCATGTATCGCCGTAAACTG
GACCGTGAAGAAATCAAGCCTGTGATGGCACTGGGCATAGTGCCTATGTGCTCCTACCAGATGGAGAGGATGTTC
AACACCACTCGGATCCCGGGCAAGGACACAGATGTGCTACAGCACCTCTCAGACAGCCGGCACGTGGCTGTCTAC
CACAAGGGACGCTTCTTCAAGCTGTGGCTCTATGAGGGCGCCCGTCTGCTCAAGCCTCAGGATCTGGAGATGCAG
TTCCAGAGGATCCTGGACGACCCCTCCCCACCTCAGCCTGGGGAGGAGAAGCTGGCAGCCCTCACTGCAGGAGGA
AGGGTGGAGTGGGCGCAGGCACGCCAGGCCTTCTTTAGCTCTGGAAAGAATAAGGCTGCCTTGGAGGCCATCGAG
CGTGCCGCTTTCTTCGTGGCCCTGGATGAGGAATCCTACTCCTATGACCCCGAAGATGAGGCCAGCCTCAGCCTC
TATGGCAAGGCCCTGCTACATGGCAACTGCTACAACAGGTGGTTTGACAAATCCTTCACTCTCATTTCCTTCAAG
AATGGCCAGTTGGGTCTCAATGCAGAGCATGCGTGGGCAGATGCTCCCATCATTGGGCACCTCTGGGAGTTTGTC
CTGGGCACAGACAGCTTCCACCTGGGCTACACGGAGACCGGGCACTGCCTGGGCAAACCGAACCCTGCGCTCGCA
CCTCCTACACGGCTGCAGTG
GGACATTCCAAAACAGTGCCAGGCGGTCATCGAGAGTTCCTACCAGGTGGCCAAGGCGTTGGCAGACGACGTGGA
GTTGTACTGCTTCCAGTTCCTGCCCTTTGGCAAAGGCCTCATCAAGAAGTGCCGGACCAGCCCTGATGCCTTTGT
GCAGATCGCGCTGCAGCTGGCTCACTTCCGGGACAGGGGTAAGTTCTGCCTGACCTATGAGGCCTCAATGACCAG
AATGTTCCGGGAGGGACGGACTGAGACTGTGCGTTCCTGTACCAGCGAGTCCACAGCCTTTGTGCAGGCCATGAT
GGAGGGGTCCCACACAAAAGCAGACCTGCGAGATCTCTTCCAGAAGGCTGCTAAGAAGCACCAGAATATGTACCG
CCTGGCCATGACCGGGGCAGGGATCGACAGGCACCTCTTCTGCCTTTACTTGGTCTCCAAGTACCTAGGAGTCAG
CTCTCCTTTCCTTGCTGAGGTGCTCTCGGAACCCTGGCGTCTCTCCACCAGCCAGATCCCCCAATCCCAGATCCG
CATGTTCGACCCAGAGCAGCACCCCAATCACCTGGGCGCTGGAGGTGGCTTTGGCCCTGTAGCAGATGATGGCTA
TGGAGTTTCCTACATGATTGCAGGCGAGAACACGATCTTCTTCCACATCTCCAGCAAGTTCTCAAGCTCAGAGAC
GAACGCCCAGCGCTTTGGAAACCACATCCGCAAAGCCCTGCTGGACATTGCTGATCTTTTCCAAGTTCCCAAGGC
CTACAGCTGAAGCCCTTAGG
TACCTGTGTTTTGTTTGGGAACTCGGAGGCCCTCCCCCTCCCCCAGCTCAGACCACAGAGGTGGCAAGAGAAGGG
CTGAAGCTGGAAGACTGTTCATGAGGGACTTGTGTGACCTGCTTTGAAATGTGTGACTCTGCTGAGTGACGTAGG
CTCTGAGATAGCTGTCCACGCCCACGTGTTTGCTTGGAATAAATACTTGCCTCAGAACCTTCA
>CPS1_NM_001145135.2 (SEQ ID NO: 20)
AGACCTTTGAGCAACCTTCACCGCACAGAAACCCAGCCGCGCCCTGCAATTCCCACCGCGGAAGGTGCCGACCAA
CCCCCAGGATGGCGGAAGCTCACCAGGCCGTGGCCTTCCAGTTCACGGTGACCCCAGACGGGGTCGACTTCCGGC
TCAGTCGGGAGGCCCTGAAACACGTCTACCTGTCTGGGATCAACTCCTGGAAGAAACGCCTGATCCGCATCAAGA
ATGGCATCCTCAGGGGCGTGTACCCTGGCAGCCCCACCAGCTGGCTGGTCGTCATCATGGCAACAGTGGGTTCCT
CCTTCTGCAACGTGGACATCTCCTTGGGGCTGGTCAGTTGCATCCAGAGATGCCTCCCTCAGGGGTGTGGCCCCT
ACCAGACCCCGCAGACCCGGGCACTTCTCAGCATGGCCATCTTCTCCACGGGCGTCTGGGTGACGGGCATCTTCT
TCTTCCGCCAAACCCTGAAGCTGCTTCTCTGCTACCATGGGTGGATGTTTGAGATGCATGGCAAGACCAGCAACT
TGACCAGGATCTGGGCTATGTGTATCCGCCTTCTATCCAGCCGGCACCCTATGCTCTACAGCTTCCAGACATCTC
TGCCCAAGCTTCCTGTGCCCAGGGTGTCAGCCACAATTCAGCGGTACCTAGAGTCTGTGCGCCCCTTGTTGGATG
ATGAGGAATATTACCGCATGGAGTTGCTGGCCAAAGAATTCCAGGACAAGACTGCCCCCAGGCTGCAGAAATACC
TGGTGCTCAAGTCATGGTGG
GCAAGTAACTATGTGAGTGACTGGTGGGAAGAGTACATCTACCTTCGAGGCAGGAGCCCTCTCATGGTGAACAGC
AACTATTATGTCATGGACCTTGTGCTCATCAAGAATACAGACGTGCAGGCAGCCCGCCTGGGAAACATCATCCAC
GCCATGATCATGTATCGCCGTAAACTGGACCGTGAAGAAATCAAGCCTGTGATGGCACTGGGCATAGTGCCTATG
TGCTCCTACCAGATGGAGAGGATGTTCAACACCACTCGGATCCCGGGCAAGGACACAGATGTGCTACAGCACCTC
TCAGACAGCCGGCACGTGGCTGTCTACCACAAGGGACGCTTCTTCAAGCTGTGGCTCTATGAGGGCGCCCGTCTG
CTCAAGCCTCAGGATCTGGAGATGCAGTTCCAGAGGATCCTGGACGACCCCTCCCCACCTCAGCCTGGGGAGGAG
AAGCTGGCAGCCCTCACTGCAGGAGGAAGGGTGGAGTGGGCGCAGGCACGCCAGGCCTTCTTTAGCTCTGGAAAG
AATAAGGCTGCCTTGGAGGCCATCGAGCGTGCCGCTTTCTTCGTGGCCCTGGATGAGGAATCCTACTCCTATGAC
CCCGAAGATGAGGCCAGCCTCAGCCTCTATGGCAAGGCCCTGCTACATGGCAACTGCTACAACAGGTGGTTTGAC
AAATCCTTCACTCTCATTTCCTTCAAGAATGGCCAGTTGGGTCTCAATGCAGAGCATGCGTGGGCAGATGCTCCC
ATCATTGGGCACCTCTGGGA
GTTTGTCCTGGGCACAGACAGCTTCCACCTGGGCTACACGGAGACCGGGCACTGCCTGGGCAAACCGAACCCTGC
GCTCGCACCTCCTACACGGCTGCAGTGGGACATTCCAAAACAGTGCCAGGCGGTCATCGAGAGTTCCTACCAGGT
GGCCAAGGCGTTGGCAGACGACGTGGAGTTGTACTGCTTCCAGTTCCTGCCCTTTGGCAAAGGCCTCATCAAGAA
GTGCCGGACCAGCCCTGATGCCTTTGTGCAGATCGCGCTGCAGCTGGCTCACTTCCGGGACAGGGGTAAGTTCTG
CCTGACCTATGAGGCCTCAATGACCAGAATGTTCCGGGAGGGACGGACTGAGACTGTGCGTTCCTGTACCAGCGA
GTCCACAGCCTTTGTGCAGGCCATGATGGAGGGGTCCCACACAAAAGCAGACCTGCGAGATCTCTTCCAGAAGGC
TGCTAAGAAGCACCAGAATATGTACCGCCTGGCCATGACCGGGGCAGGGATCGACAGGCACCTCTTCTGCCTTTA
CTTGGTCTCCAAGTACCTAGGAGTCAGCTCTCCTTTCCTTGCTGAGGTGCTCTCGGAACCCTGGCGTCTCTCCAC
CAGCCAGATCCCCCAATCCCAGATCCGCATGTTCGACCCAGAGCAGCACCCCAATCACCTGGGCGCTGGAGGTGG
CTTTGGCCCTGTAGCAGATGATGGCTATGGAGTTTCCTACATGATTGCAGGCGAGAACACGATCTTCTTCCACAT
CTCCAGCAAGTTCTCAAGCT
CAGAGACGAACGCCCAGCGCTTTGGAAACCACATCCGCAAAGCCCTGCTGGACATTGCTGATCTTTTCCAAGTTC
CCAAGGCCTACAGCTGAAGCCCTTAGGTACCTGTGTTTTGTTTGGGAACTCGGAGGCCCTCCCCCTCCCCCAGCT
CAGACCACAGAGGTGGCAAGAGAAGGGCTGAAGCTGGAAGACTGTTCATGAGGGACTTGTGTGACCTGCTTTGAA
ATGTGTGACTCTGCTGAGTGACGTAGGCTCTGAGATAGCTGTCCACGCCCACGTGTTTGCTTGGAATAAATACTT
GCCTCAGAACCTTCA
>CPS1_NM_001145137.2 (SEQ ID NO: 21)
GCGGACTGGCTGGGGGCGTCTCGGCGCGGCTGGCGGCGGGGCCGGCCTAAGCGCGCCCGCGCACCCATCTGCCCC
CGTCCTAGGTGCCGACCAACCCCCAGGATGGCGGAAGCTCACCAGGCCGTGGCCTTCCAGTTCACGGTGACCCCA
GACGGGGTCGACTTCCGGCTCAGTCGGGAGGCCCTGAAACACGTCTACCTGTCTGGGATCAACTCCTGGAAGAAA
CGCCTGATCCGCATCAAGAATGGCATCCTCAGGGGCGTGTACCCTGGCAGCCCCACCAGCTGGCTGGTCGTCATC
ATGGCAACAGTGGGTTCCTCCTTCTGCAACGTGGACATCTCCTTGGGGCTGGTCAGTTGCATCCAGAGATGCCTC
CCTCAGGGGTGTGGCCCCTACCAGACCCCGCAGACCCGGGCACTTCTCAGCATGGCCATCTTCTCCACGGGCGTC
TGGGTGACGGGCATCTTCTTCTTCCGCCAAACCCTGAAGCTGCTTCTCTGCTACCATGGGTGGATGTTTGAGATG
CATGGCAAGACCAGCAACTTGACCAGGATCTGGGCTATGTGTATCCGCCTTCTATCCAGCCGGCACCCTATGCTC
TACAGCTTCCAGACATCTCTGCCCAAGCTTCCTGTGCCCAGGGTGTCAGCCACAATTCAGCGGTACCTAGAGTCT
GTGCGCCCCTTGTTGGATGATGAGGAATATTACCGCATGGAGTTGCTGGCCAAAGAATTCCAGGACAAGACTGCC
CCCAGGCTGCAGAAATACCT
GGTGCTCAAGTCATGGTGGGCAAGTAACTATGTGAGTGACTGGTGGGAAGAGTACATCTACCTTCGAGGCAGGAG
CCCTCTCATGGTGAACAGCAACTATTATGTCATGGACCTTGTGCTCATCAAGAATACAGACGTGCAGGCAGCCCG
CCTGGGAAACATCATCCACGCCATGATCATGTATCGCCGTAAACTGGACCGTGAAGAAATCAAGCCTGTGATGGC
ACTGGGCATAGTGCCTATGTGCTCCTACCAGATGGAGAGGATGTTCAACACCACTCGGATCCCGGGCAAGGACAC
AGATGTGCTACAGCACCTCTCAGACAGCCGGCACGTGGCTGTCTACCACAAGGGACGCTTCTTCAAGCTGTGGCT
CTATGAGGGCGCCCGTCTGCTCAAGCCTCAGGATCTGGAGATGCAGTTCCAGAGGATCCTGGACGACCCCTCCCC
ACCTCAGCCTGGGGAGGAGAAGCTGGCAGCCCTCACTGCAGGAGGAAGGGTGGAGTGGGCGCAGGCACGCCAGGC
CTTCTTTAGCTCTGGAAAGAATAAGGCTGCCTTGGAGGCCATCGAGCGTGCCGCTTTCTTCGTGGCCCTGGATGA
GGAATCCTACTCCTATGACCCCGAAGATGAGGCCAGCCTCAGCCTCTATGGCAAGGCCCTGCTACATGGCAACTG
CTACAACAGGTGGTTTGACAAATCCTTCACTCTCATTTCCTTCAAGAATGGCCAGTTGGGTCTCAATGCAGAGCA
TGCGTGGGCAGATGCTCCCA
TCATTGGGCACCTCTGGGAGTTTGTCCTGGGCACAGACAGCTTCCACCTGGGCTACACGGAGACCGGGCACTGCC
TGGGCAAACCGAACCCTGCGCTCGCACCTCCTACACGGCTGCAGTGGGACATTCCAAAACAGTGCCAGGCGGTCA
TCGAGAGTTCCTACCAGGTGGCCAAGGCGTTGGCAGACGACGTGGAGTTGTACTGCTTCCAGTTCCTGCCCTTTG
GCAAAGGCCTCATCAAGAAGTGCCGGACCAGCCCTGATGCCTTTGTGCAGATCGCGCTGCAGCTGGCTCACTTCC
GGGACAGGGGTAAGTTCTGCCTGACCTATGAGGCCTCAATGACCAGAATGTTCCGGGAGGGACGGACTGAGACTG
TGCGTTCCTGTACCAGCGAGTCCACAGCCTTTGTGCAGGCCATGATGGAGGGGTCCCACACAAAAGCAGACCTGC
GAGATCTCTTCCAGAAGGCTGCTAAGAAGCACCAGAATATGTACCGCCTGGCCATGACCGGGGCAGGGATCGACA
GGCACCTCTTCTGCCTTTACTTGGTCTCCAAGTACCTAGGAGTCAGCTCTCCTTTCCTTGCTGAGGTGCTCTCGG
AACCCTGGCGTCTCTCCACCAGCCAGATCCCCCAATCCCAGATCCGCATGTTCGACCCAGAGCAGCACCCCAATC
ACCTGGGCGCTGGAGGTGGCTTTGGCCCTGTAGCAGATGATGGCTATGGAGTTTCCTACATGATTGCAGGCGAGA
ACACGATCTTCTTCCACATC
TCCAGCAAGTTCTCAAGCTCAGAGACGAACGCCCAGCGCTTTGGAAACCACATCCGCAAAGCCCTGCTGGACATT
GCTGATCTTTTCCAAGTTCCCAAGGCCTACAGCTGAAGCCCTTAGGTACCTGTGTTTTGTTTGGGAACTCGGAGG
CCCTCCCCCTCCCCCAGCTCAGACCACAGAGGTGGCAAGAGAAGGGCTGAAGCTGGAAGACTGTTCATGAGGGAC
TTGTGTGACCTGCTTTGAAATGTGTGACTCTGCTGAGTGACGTAGGCTCTGAGATAGCTGTCCACGCCCACGTGT
TTGCTTGGAATAAATACTTGCCTCAGAACCTTCA
>CPS1_NM_004377.4 (SEQ ID NO: 22)
AGAGTGGCTGGCCCCACGCACGGACAGGAGTGAACCCGAGCTGTGCCGACCAACCCCCAGGATGGCGGAAGCTCA
CCAGGCCGTGGCCTTCCAGTTCACGGTGACCCCAGACGGGGTCGACTTCCGGCTCAGTCGGGAGGCCCTGAAACA
CGTCTACCTGTCTGGGATCAACTCCTGGAAGAAACGCCTGATCCGCATCAAGAATGGCATCCTCAGGGGCGTGTA
CCCTGGCAGCCCCACCAGCTGGCTGGTCGTCATCATGGCAACAGTGGGTTCCTCCTTCTGCAACGTGGACATCTC
CTTGGGGCTGGTCAGTTGCATCCAGAGATGCCTCCCTCAGGGGTGTGGCCCCTACCAGACCCCGCAGACCCGGGC
ACTTCTCAGCATGGCCATCTTCTCCACGGGCGTCTGGGTGACGGGCATCTTCTTCTTCCGCCAAACCCTGAAGCT
GCTTCTCTGCTACCATGGGTGGATGTTTGAGATGCATGGCAAGACCAGCAACTTGACCAGGATCTGGGCTATGTG
TATCCGCCTTCTATCCAGCCGGCACCCTATGCTCTACAGCTTCCAGACATCTCTGCCCAAGCTTCCTGTGCCCAG
GGTGTCAGCCACAATTCAGCGGTACCTAGAGTCTGTGCGCCCCTTGTTGGATGATGAGGAATATTACCGCATGGA
GTTGCTGGCCAAAGAATTCCAGGACAAGACTGCCCCCAGGCTGCAGAAATACCTGGTGCTCAAGTCATGGTGGGC
AAGTAACTATGTGAGTGACT
GGTGGGAAGAGTACATCTACCTTCGAGGCAGGAGCCCTCTCATGGTGAACAGCAACTATTATGTCATGGACCTTG
TGCTCATCAAGAATACAGACGTGCAGGCAGCCCGCCTGGGAAACATCATCCACGCCATGATCATGTATCGCCGTA
AACTGGACCGTGAAGAAATCAAGCCTGTGATGGCACTGGGCATAGTGCCTATGTGCTCCTACCAGATGGAGAGGA
TGTTCAACACCACTCGGATCCCGGGCAAGGACACAGATGTGCTACAGCACCTCTCAGACAGCCGGCACGTGGCTG
TCTACCACAAGGGACGCTTCTTCAAGCTGTGGCTCTATGAGGGCGCCCGTCTGCTCAAGCCTCAGGATCTGGAGA
TGCAGTTCCAGAGGATCCTGGACGACCCCTCCCCACCTCAGCCTGGGGAGGAGAAGCTGGCAGCCCTCACTGCAG
GAGGAAGGGTGGAGTGGGCGCAGGCACGCCAGGCCTTCTTTAGCTCTGGAAAGAATAAGGCTGCCTTGGAGGCCA
TCGAGCGTGCCGCTTTCTTCGTGGCCCTGGATGAGGAATCCTACTCCTATGACCCCGAAGATGAGGCCAGCCTCA
GCCTCTATGGCAAGGCCCTGCTACATGGCAACTGCTACAACAGGTGGTTTGACAAATCCTTCACTCTCATTTCCT
TCAAGAATGGCCAGTTGGGTCTCAATGCAGAGCATGCGTGGGCAGATGCTCCCATCATTGGGCACCTCTGGGAGT
TTGTCCTGGGCACAGACAGC
TTCCACCTGGGCTACACGGAGACCGGGCACTGCCTGGGCAAACCGAACCCTGCGCTCGCACCTCCTACACGGCTG
CAGTGGGACATTCCAAAACAGTGCCAGGCGGTCATCGAGAGTTCCTACCAGGTGGCCAAGGCGTTGGCAGACGAC
GTGGAGTTGTACTGCTTCCAGTTCCTGCCCTTTGGCAAAGGCCTCATCAAGAAGTGCCGGACCAGCCCTGATGCC
TTTGTGCAGATCGCGCTGCAGCTGGCTCACTTCCGGGACAGGGGTAAGTTCTGCCTGACCTATGAGGCCTCAATG
ACCAGAATGTTCCGGGAGGGACGGACTGAGACTGTGCGTTCCTGTACCAGCGAGTCCACAGCCTTTGTGCAGGCC
ATGATGGAGGGGTCCCACACAAAAGCAGACCTGCGAGATCTCTTCCAGAAGGCTGCTAAGAAGCACCAGAATATG
TACCGCCTGGCCATGACCGGGGCAGGGATCGACAGGCACCTCTTCTGCCTTTACTTGGTCTCCAAGTACCTAGGA
GTCAGCTCTCCTTTCCTTGCTGAGGTGCTCTCGGAACCCTGGCGTCTCTCCACCAGCCAGATCCCCCAATCCCAG
ATCCGCATGTTCGACCCAGAGCAGCACCCCAATCACCTGGGCGCTGGAGGTGGCTTTGGCCCTGTAGCAGATGAT
GGCTATGGAGTTTCCTACATGATTGCAGGCGAGAACACGATCTTCTTCCACATCTCCAGCAAGTTCTCAAGCTCA
GAGACGAACGCCCAGCGCTT
TGGAAACCACATCCGCAAAGCCCTGCTGGACATTGCTGATCTTTTCCAAGTTCCCAAGGCCTACAGCTGAAGGTT
GGAGAAATGCCAGCTGCCCTTTCGTCCCCACACTGTGGAGGAAGGGACCTGTGGCAGCTCACAGGCATGAGGGGT
GGCCGTGCACAGGTGCCCAGGCTCCAAGGACAGCTCCGGCAGCAGGTCCTCGCTGGGCAGATGCTGCTCCCTGAG
GGCCCAGGTGGTGGAGGTGGGGTTGGAGCAGGAAGGGAATTTTGATTTTTTTTTTTCTTGATAGATACTAATAAA
AATAAGGCTGTGTAATTTTCTCTCAGCCCTTAGGTACCTGTGTTTTGTTTGGGAACTCGGAGGCCCTCCCCCTCC
CCCAGCTCAGACCACAGAGGTGGCAAGAGAAGGGCTGAAGCTGGAAGACTGTTCATGAGGGACTTGTGTGACCTG
CTTTGAAATGTGTGACTCTGCTGAGTGACGTAGGCTCTGAGATAGCTGTCCACGCCCACGTGTTTGCTTGGAATA
AATACTTGCCTCAGAACCTTCA
>CPS1_NM_152245.3 (SEQ ID NO: 23)
AGACCTTTGAGCAACCTTCACCGCACAGAAACCCAGCCGCGCCCTGCAATTCCCACCGCGGAAGGTGCCGACCAA
CCCCCAGGATGGCGGAAGCTCACCAGGCCGTGGCCTTCCAGTTCACGGTGACCCCAGACGGGGTCGACTTCCGGC
TCAGTCGGGAGGCCCTGAAACACGTCTACCTGTCTGGGATCAACTCCTGGAAGAAACGCCTGATCCGCATCAAGA
ATGGCATCCTCAGGGGCGTGTACCCTGGCAGCCCCACCAGCTGGCTGGTCGTCATCATGGCAACAGTGGGTTCCT
CCTTCTGCAACGTGGACATCTCCTTGGGGCTGGTCAGTTGCATCCAGAGATGCCTCCCTCAGGGGTGTGGCCCCT
ACCAGACCCCGCAGACCCGGGCACTTCTCAGCATGGCCATCTTCTCCACGGGCGTCTGGGTGACGGGCATCTTCT
TCTTCCGCCAAACCCTGAAGCTGCTTCTCTGCTACCATGGGTGGATGTTTGAGATGCATGGCAAGACCAGCAACT
TGACCAGGATCTGGGCTATGTGTATCCGCCTTCTATCCAGCCGGCACCCTATGCTCTACAGCTTCCAGACATCTC
TGCCCAAGCTTCCTGTGCCCAGGGTGTCAGCCACAATTCAGCGGTACCTAGAGTCTGTGCGCCCCTTGTTGGATG
ATGAGGAATATTACCGCATGGAGTTGCTGGCCAAAGAATTCCAGGACAAGACTGCCCCCAGGCTGCAGAAATACC
TGGTGCTCAAGTCATGGTGG
GCAAGTAACTATGTGAGTGACTGGTGGGAAGAGTACATCTACCTTCGAGGCAGGAGCCCTCTCATGGTGAACAGC
AACTATTATGTCATGGACCTTGTGCTCATCAAGAATACAGACGTGCAGGCAGCCCGCCTGGGAAACATCATCCAC
GCCATGATCATGTATCGCCGTAAACTGGACCGTGAAGAAATCAAGCCTGTGATGGCACTGGGCATAGTGCCTATG
TGCTCCTACCAGATGGAGAGGATGTTCAACACCACTCGGATCCCGGGCAAGGACACAGATGTGCTACAGCACCTC
TCAGACAGCCGGCACGTGGCTGTCTACCACAAGGGACGCTTCTTCAAGCTGTGGCTCTATGAGGGCGCCCGTCTG
CTCAAGCCTCAGGATCTGGAGATGCAGTTCCAGAGGATCCTGGACGACCCCTCCCCACCTCAGCCTGGGGAGGAG
AAGCTGGCAGCCCTCACTGCAGGAGGAAGGGTGGAGTGGGCGCAGGCACGCCAGGCCTTCTTTAGCTCTGGAAAG
AATAAGGCTGCCTTGGAGGCCATCGAGCGTGCCGCTTTCTTCGTGGCCCTGGATGAGGAATCCTACTCCTATGAC
CCCGAAGATGAGGCCAGCCTCAGCCTCTATGGCAAGGCCCTGCTACATGGCAACTGCTACAACAGGTGGTTTGAC
AAATCCTTCACTCTCATTTCCTTCAAGAATGGCCAGTTGGGTCTCAATGCAGAGCATGCGTGGGCAGATGCTCCC
ATCATTGGGCACCTCTGGGA
GTTTGTCCTGGGCACAGACAGCTTCCACCTGGGCTACACGGAGACCGGGCACTGCCTGGGCAAACCGAACCCTGC
GCTCGCACCTCCTACACGGCTGCAGTGGGACATTCCAAAACAGTGCCAGGCGGTCATCGAGAGTTCCTACCAGGT
GGCCAAGGCGTTGGCAGACGACGTGGAGTTGTACTGCTTCCAGTTCCTGCCCTTTGGCAAAGGCCTCATCAAGAA
GTGCCGGACCAGCCCTGATGCCTTTGTGCAGATCGCGCTGCAGCTGGCTCACTTCCGGGACAGGGGTAAGTTCTG
CCTGACCTATGAGGCCTCAATGACCAGAATGTTCCGGGAGGGACGGACTGAGACTGTGCGTTCCTGTACCAGCGA
GTCCACAGCCTTTGTGCAGGCCATGATGGAGGGGTCCCACACAAAAGCAGACCTGCGAGATCTCTTCCAGAAGGC
TGCTAAGAAGCACCAGAATATGTACCGCCTGGCCATGACCGGGGCAGGGATCGACAGGCACCTCTTCTGCCTTTA
CTTGGTCTCCAAGTACCTAGGAGTCAGCTCTCCTTTCCTTGCTGAGGTGCTCTCGGAACCCTGGCGTCTCTCCAC
CAGCCAGATCCCCCAATCCCAGATCCGCATGTTCGACCCAGAGCAGCACCCCAATCACCTGGGCGCTGGAGGTGG
CTTTGGCCCTGTAGCAGATGATGGCTATGGAGTTTCCTACATGATTGCAGGCGAGAACACGATCTTCTTCCACAT
CTCCAGCAAGTTCTCAAGCT
CAGAGACGAACGCCCAGCGCTTTGGAAACCACATCCGCAAAGCCCTGCTGGACATTGCTGATCTTTTCCAAGTTC
CCAAGGCCTACAGCTGAAGGTTGGAGAAATGCCAGCTGCCCTTTCGTCCCCACACTGTGGAGGAAGGGACCTGTG
GCAGCTCACAGGCATGAGGGGTGGCCGTGCACAGGTGCCCAGGCTCCAAGGACAGCTCCGGCAGCAGGTCCTCGC
TGGGCAGATGCTGCTCCCTGAGGGCCCAGGTGGTGGAGGTGGGGTTGGAGCAGGAAGGGAATTTTGATTTTTTTT
TTTCTTGATAGATACTAATAAAAATAAGGCTGTGTAATTTTCTCTCAGCCCTTAGGTACCTGTGTTTTGTTTGGG
AACTCGGAGGCCCTCCCCCTCCCCCAGCTCAGACCACAGAGGTGGCAAGAGAAGGGCTGAAGCTGGAAGACTGTT
CATGAGGGACTTGTGTGACCTGCTTTGAAATGTGTGACTCTGCTGAGTGACGTAGGCTCTGAGATAGCTGTCCAC
GCCCACGTGTTTGCTTGGAATAAATACTTGCCTCAGAACCTTCA
>CPS1_NM_152246.3 (SEQ ID NO: 24)
AGAGTGGCTGGCCCCACGCACGGACAGGAGTGAACCCGAGCTGTGCCGACCAACCCCCAGGATGGCGGAAGCTCA
CCAGGCCGTGGCCTTCCAGTTCACGGTGACCCCAGACGGGGTCGACTTCCGGCTCAGTCGGGAGGCCCTGAAACA
CGTCTACCTGTCTGGGATCAACTCCTGGAAGAAACGCCTGATCCGCATCAAGAATGGCATCCTCAGGGGCGTGTA
CCCTGGCAGCCCCACCAGCTGGCTGGTCGTCATCATGGCAACAGTGGGTTCCTCCTTCTGCAACGTGGACATCTC
CTTGGGGCTGGTCAGTTGCATCCAGAGATGCCTCCCTCAGGGGTGTGGCCCCTACCAGACCCCGCAGACCCGGGC
ACTTCTCAGCATGGCCATCTTCTCCACGGGCGTCTGGGTGACGGGCATCTTCTTCTTCCGCCAAACCCTGAAGCT
GCTTCTCTGCTACCATGGGTGGATGTTTGAGATGCATGGCAAGACCAGCAACTTGACCAGGATCTGGGCTATGTG
TATCCGCCTTCTATCCAGCCGGCACCCTATGCTCTACAGCTTCCAGACATCTCTGCCCAAGCTTCCTGTGCCCAG
GGTGTCAGCCACAATTCAGCGGTACCTAGAGTCTGTGCGCCCCTTGTTGGATGATGAGGAATATTACCGCATGGA
GTTGCTGGCCAAAGAATTCCAGGACAAGACTGCCCCCAGGCTGCAGAAATACCTGGTGCTCAAGTCATGGTGGGC
AAGTAACTATGTGAGTGACT
GGTGGGAAGAGTACATCTACCTTCGAGGCAGGAGCCCTCTCATGGTGAACAGCAACTATTATGTCATGGACCTTG
TGCTCATCAAGAATACAGACGTGCAGGCAGCCCGCCTGGGAAACATCATCCACGCCATGATCATGTATCGCCGTA
AACTGGACCGTGAAGAAATCAAGCCTGTGATGGCACTGGGCATAGTGCCTATGTGCTCCTACCAGATGGAGAGGA
TGTTCAACACCACTCGGATCCCGGGCAAGGACACAGATGTGCTACAGCACCTCTCAGACAGCCGGCACGTGGCTG
TCTACCACAAGGGACGCTTCTTCAAGCTGTGGCTCTATGAGGGCGCCCGTCTGCTCAAGCCTCAGGATCTGGAGA
TGCAGTTCCAGAGGATCCTGGACGACCCCTCCCCACCTCAGCCTGGGGAGGAGAAGCTGGCAGCCCTCACTGCAG
GAGGAAGGGTGGAGTGGGCGCAGGCACGCCAGGCCTTCTTTAGCTCTGGAAAGAATAAGGCTGCCTTGGAGGCCA
TCGAGCGTGCCGCTTTCTTCGTGGCCCTGGATGAGGAATCCTACTCCTATGACCCCGAAGATGAGGCCAGCCTCA
GCCTCTATGGCAAGGCCCTGCTACATGGCAACTGCTACAACAGGTGGTTTGACAAATCCTTCACTCTCATTTCCT
TCAAGAATGGCCAGTTGGGTCTCAATGCAGAGCATGCGTGGGCAGATGCTCCCATCATTGGGCACCTCTGGGAGT
TTGTCCTGGGCACAGACAGC
TTCCACCTGGGCTACACGGAGACCGGGCACTGCCTGGGCAAACCGAACCCTGCGCTCGCACCTCCTACACGGCTG
CAGTGGGACATTCCAAAACAGTGCCAGGCGGTCATCGAGAGTTCCTACCAGGTGGCCAAGGCGTTGGCAGACGAC
GTGGAGTTGTACTGCTTCCAGTTCCTGCCCTTTGGCAAAGGCCTCATCAAGAAGTGCCGGACCAGCCCTGATGCC
TTTGTGCAGATCGCGCTGCAGCTGGCTCACTTCCGGGACAGGGGTAAGTTCTGCCTGACCTATGAGGCCTCAATG
ACCAGAATGTTCCGGGAGGGACGGACTGAGACTGTGCGTTCCTGTACCAGCGAGTCCACAGCCTTTGTGCAGGCC
ATGATGGAGGGGTCCCACACAAAAGCAGACCTGCGAGATCTCTTCCAGAAGGCTGCTAAGAAGCACCAGAATATG
TACCGCCTGGCCATGACCGGGGCAGGGATCGACAGGCACCTCTTCTGCCTTTACTTGGTCTCCAAGTACCTAGGA
GTCAGCTCTCCTTTCCTTGCTGAGGTGCTCTCGGAACCCTGGCGTCTCTCCACCAGCCAGATCCCCCAATCCCAG
ATCCGCATGTTCGACCCAGAGCAGCACCCCAATCACCTGGGCGCTGGAGGTGGCTTTGGCCCTGTAGCAGATGAT
GGCTATGGAGTTTCCTACATGATTGCAGGCGAGAACACGATCTTCTTCCACATCTCCAGCAAGTTCTCAAGCTCA
GAGACGAACGCCCAGCGCTT
TGGAAACCACATCCGCAAAGCCCTGCTGGACATTGCTGATCTTTTCCAAGTTCCCAAGGCCTACAGCTGAAGCCC
TTAGGTACCTGTGTTTTGTTTGGGAACTCGGAGGCCCTCCCCCTCCCCCAGCTCAGACCACAGAGGTGGCAAGAG
AAGGGCTGAAGCTGGAAGACTGTTCATGAGGGACTTGTGTGACCTGCTTTGAAATGTGTGACTCTGCTGAGTGAC
GTAGGCTCTGAGATAGCTGTCCACGCCCACGTGTTTGCTTGGAATAAATACTTGCCTCAGAACCTTCA
H1 Sequences:
>Aardvark_H1_Bidirectional_Promoter (SEQ ID NO: 25)
GGAACGAAACTAACTTGGCCAAACTATATAAGAATGCCATAGCTTTCAACATTTAATGGTTAGGGTGCCTTCTCA
TAATACACAGCGACATGCAAATATCATGGCCCTTCCAGGAGGCGTGCCTCCCCGTCCCGCGTGTGCGTCTTGCTT
GTGCGCAGGCGCGCTGCTCTTCCGGCTGTAAGACTTTGAGCCCTTGATTTCTGTGAGCGGGTTCGTGAAGTCAGT
GTTCTGGCTCC
>Angolan_colobus_H1_Bidirectional_Promoter (SEQ ID NO: 26)
GGGGAAGGGTGGTCCTCCATAGAACTTATAAGACTCCCATACTCAAAGACATTTCACGTTTATGGTGACTTCCCA
GAAGACATAGCGACATGCAAATATTGCAGGGCGTCACACCCCCTGTCCCTTACAGCTCTCTTCCTGCCAGGGCGC
ACGCGCGCTGGGTGTTCCCGCGTAGTGACACTGGGCCCGCGATTCCTTGGAACGGGTTGATGACGTCAGCGTTCG
AATTAC
>Big_brown_bat_H1_Bidirectional_Promoter (SEQ ID NO: 27)
GGGAAGCGAGCGTCACACGGCGGATATATAAGGCCCCCTTACCTGAAGGCCTTTTACGGTTAGGGTGACTTCCCA
CAACACTTAGCGACATGCAAATTTAGACGGGCGTGCCTCCCCGTCCCTGGGCAACTTCTCTCCTGGACACGCGCG
CTCGCGCTGAGTGTTCCCGCCTTTTGACTCCAGCCGAGCGAATCCTGGGAGAGGGCAGATGACGTCAACAGTCAG
GCTCC
>Black_flying-fox_H1_Bidirectional_Promoter (SEQ ID NO: 28)
GAGAGAAAAAGCCTGCACGCAGAATATATAAGGATCCCATATCTGAAGACATTTTACGGTTACGGTGATTTCCCA
CAACACATAGCGACATGTAAATATAGTGGGGCATGCCTCTCCTGTCCCTGGGCAGCTTCTCGCCAGAACGCACGC
GCGGTGCGTGTTCCCGCCTTGTGACTAAGTTGGCGAGTCAGGGAGGAGATTGATGATGTCATCATCGTCAGCTCA
CCCGCTCC
>Black_snub-nosed_monkey_H1_Bidirectional_Promoter (SEQ ID NO: 29)
GGGGAAGGGTGGTCCTACACAGAGCTTATAAGACTCCCATACTCAAAGACATTTCTCGTTTATGGTGACTTCCCA
GAAGCCATAGCGACATGCAAATATTGCAGGGCGTCACACCCCTGTCCCTTACAGCCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
CTTCC
>Bonobo_H1_Bidirectional_Promoter (SEQ ID NO: 30)
GGGAAAGGGTGGTGCCACACAGAACTTATAAGACTCCCATATCCAAAGACATTTCACGTTTATGGTGATTTCCCA
GAACACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Brush-tailed_rat_H1_Bidirectional_Promoter (SEQ ID NO: 31)
GAAGGAAGTTAGTCACAAACGCAAATTATAAGAGGTCCAAAGCTCAGTGTACTCTATGGTTAGGGTGACTTCCCA
CAATACATAGCGATATGCAGATTTCTTCCCCAGTCTGGCCCGCTGGGCCCTCCCTAGAGCGCATGCGCTGCAAGT
CCACGGCGGAGCACCGGGCGGGCGATCCCGGAGCGGGTTGATGACGTCAGCGTTTGAACTCC
>Camel_H1_Bidirectional_Promoter (SEQ ID NO: 32)
GAGAAAGGGTGGGCTCACGCCACCTTTATAAGGCTCCCAAACTTAAAGACATTTCTCGGTTATGGCGACTTCCCA
CAACACATAGCGACATGCAAATACTGCAGACCTGTGGCGCCGACCCGGTCCTGTGCAGCCATCTTTAAGGCTGGG
ACGCACGCGCGCTGCGTGTTCCCGCCCTGTGACTGCGCCGGCGATTACTGGGAGAGGATTGATGACGTCAACGTT
CGGGTTCC
>Cape_golden_mole_H1_Bidirectional_Promoter (SEQ ID NO: 33)
GGGCTAACACTGTGTTGGTATTAGCTTATAAGAAACCCAAATATAAAGTCATTTAACGCTTAGTGTGACTTCCCA
TCATACAAAGCGACATGCAAATATCATGGGCCTTCCGGGAGGCGTGCCTTCCCGTCCTGCGTACTGGAGTTCTCT
CTGGGGCGCACGCGCGCTATGTGTTTCCCGCCTTGTGACTTAGGGCGGGCGATTCCTGAGATCCGAATGGTGACG
TCAACTTTCAGGCTCG
>Chinchilla_H1_Bidirectional_Promoter (SEQ ID NO: 34)
GAAAGCCGAAGGTTTGGAGCGAAACTTATAAGAAGCCCAAATCTCACTATATTTTTAGGTCATGGCGACTTCCCA
CAAGCCACAGCGATATGTAGATATAGGAGCCCCTCCCAGTTCTGGTCCTTCCGCGTCTCACTAAAGCGCATGCGC
TGCAGGTTCGCGGCCTGCGACTGGGCCTGCAATTCCTGGGAGCGAGTTGATGACGTCAGCGTTTGAACTCC
>Chinese_hamster_H1_Bidirectional_Promoter (SEQ ID NO: 35)
ACAGCCTGGTGAATGGCGGGCTTTATAAGGCTCCGGAGAGAAAGCGCTTTCTCAGTTATGGTGGTTTCCCACAAG
GCACAGCGCACACTTTATTTGCATGCGATCTAGCGCAGGCTCCCGCTCCAGACAAGAAGCCCGCGCTTTTCGGCT
GCTTATGATGACGTCGGGCCTCAAGCGCC
>Chinese_tree_shrew_H1_Bidirectional_Promoter (SEQ ID NO: 36)
GGGGGAAGCTGGGTCCACTGAGTTCTTATAAGGTTTCCAGTCCTAGAGCGATTTTACCATTACGGTGATTTCCCA
GCATCCGTAGCTACATGCAAATAGCGCGGGGCGCGTCTCTCAGGTCCCTCCCCCGTGCCCTCTCACTGTACGTAC
CCGCGTCCTAGGGACGCCGCGCCCGGGGTTCCCGGACGTCAGCGTTCCGACGCA
>Consensus-1_H1_Bidirectional_Promoter (SEQ ID NO: 37)
GGGGAAGGGTGGTCCCACACAGAACTTATAAGGCTCCCATACCTAAAGACATTTCACGGTTATGGTGACTTCCCA
CAAGACATAGCGACATGCAAATATTGCAGGGCGTCCCTCCCCTGTCCCTAGGCATCTTCTCGCCAGGGCGCACGC
GCGCTGCGTGTTCCCGCCTTGTGACACTGGGCCCGCGATTCCTGGGAGCGGGTTGATGACGTCAGCGTTCGAGCT
CC
>David's_myotis_H1_Bidirectional_Promoter (SEQ ID NO: 38)
GAGAGGGGCTGTGCACACGGCGGATATATAAGGCCCCCTTATGAATAACCCTTTATAAGTTATGGTGATTTCCCA
CAACGCATAGCGACATGCAAATTCGATGGGCGTGCCTCCTCTGTCCCCAGGCAACTTCTCTCCTGGACGCGCGCT
CCTCGCTGCGTGTTCCCGCCTTTTGACTCCAGCCGAGCGAATCCTGGGAGAGGGCAGGTGACGTCAACAGTCAGG
CTCG
>Drill_H1_Bidirectional_Promoter (SEQ ID NO: 39)
GGGGAAAGGTGGTCCCACACAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGATGTTCCCGCGTAGTGACCCTGGGCCCGCGATTCCTTGGAGCGGGTTGATGACGTCAGCGTTCGA
ATTCC
>Gibbon_H1_Bidirectional_Promoter (SEQ ID NO: 40)
GGGGAAAAGTAGTTTTTTTTAGACCTTATAAGATTCCCAAACCCAAAGACATTTCTCGTTTATGGTGACTTCCCA
GAAGACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTCGGCCCGCGATTCCTTGGAGCGGGTTGATGACGTCAGCGTTCGA
ATTCC
>Goat_H1_Bidirectional_Promoter (SEQ ID NO: 41)
GGCAAACAACGCGCGCAAACAGCATTTATAATGAGCTCATACCTAAAGCCACTTTACGATTACGGTGACTTCCCA
CAAGACATTGCGGCATGCAAATATTTTAGTGCGTCCCGCCCCTGGTAGTTCCACGCTAGGACGCACACGCACTAC
GGTTCCCGCCTTTAGACTGCGCTGGCGATTCCAGCGAGCGGACTGATGACGTCAGCGTTGGGGCTCC
>Golden_hamster_H1_Bidirectional_Promoter (SEQ ID NO: 42)
GTGGCCCGGCGGCGGGCGAACTATATAAGCCTCCGCGGAGGAAGCGCTTTCTCGGTTAGGGTGGTTTCCCACAAG
CCTCAGCGCACAGCCTCTTTGCATACGCTCCCGCCGCCCCCGGGCTCCTCCCTCTCCGCACAAGAAGCCCGCGCA
TTTCGACTGCGGATGATGACGTCGGGCCTCGAGCGCC
>Golden_snub-nosed_monkey_H1_Bidirectional_Promoter (SEQ ID NO: 43)
GGGGAAGGGTGGTCCTACACAGAGCTTATAAGACTCCCATACTCAAAGACATTTCTCGTTTATGGTGACTTCCCA
GAAGCCATAGCGACATGCAAATATTGCAGGGCGTCACACCCCTGTCCCTTACAGCCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Hedgehog_H1_Bidirectional_Promoter (SEQ ID NO: 44)
GCCTAAACCGGCTCTTTCAACAGACTTATAAGGACCTCTTATCTTAGGACATTTTTTTCTTAGGGTAACTTCCCA
TGATGCACAGCGATATGTAAATATGGCGCCGCGAGTCTCTCCTAGGCGTCTCCCCAGGACGCAGGCGCACTGCTT
GTTCCCGCGTTAACATTGCTGATTCTGGGAGACTGCTGATGACGTCAGCGTCCAGTCTAC
>Killer_whale_H1_Bidirectional_Promoter (SEQ ID NO: 45)
GCCGAAAACCAGGCTCAAGCCACATTTATAAGGCTCCCAAACCTAAATACATTTGTCGGTTATGGTGACTTCCCG
CAACACATTGCGACATGCAAATACTGCGGAGCGTTCCTCCCCTAGCAACTCCTCGCTGGGACGCACGCGCGCTAC
GTGCTCCCGCCTTTTGACCGAGCCGGCGATACTTGGGAGAGGGTTGATGACGTCAGCGTTCTGGCTCC
>Lesser_Egyptian_jerboa_H1_Bidirectional_Promoter (SEQ ID NO: 46)
GGGCAGACCTTAACCAAGCGGAGGTTTATAAAGCGCCCACATTCAGTGACACTTCTCAGTCACGGTGACTTCCCA
CAAAACACAGCGCATGCAAATATTATGGCGGGAGGGGGGGTGCTCGCCTGGGCGCACGCGCGCTGTGGGTTCCCG
CGAGCGGGATGATGACGTCACTAAGTGAGC
>Manatee_H1_Bidirectional_Promoter (SEQ ID NO: 47)
GAGCCAAACAGCTGTTGGTCACATTATATAAGAATCCCATATATAAAGACATTTTTGGCGTAGGGTGACTTCCCA
CAATACATAGCGACATGCAAATACCATGGTCCTCCAGGAGGCGTGCCTCCCCGTCCCCTTGGTCCGGTTCTTGCT
GGGGCGCACGCGCGCTGCGTGTTCCCGGTCTGTGACTCAGCTCGCGATTCCGGAGAGCGGATTGGTGAAGTCAAT
GTTCTGGGTCC
>Mas_night_monkey_H1_Bidirectional_Promoter (SEQ ID NO: 48)
GGGGAAGGGTGGTCCTATACAGAACTTATAAGACTCCCATACCCAAAGACATTTCACGGTTATGGTGACTTCCCA
GAAGACACAGCGACATGCAAATATTGTAGGTCGTGCCTCGCTTGTCCCTCAGTAGTCTTCCTTTCAGAGCGCACG
CGCGCTGGGTGTCCCGCCAACTGACACTGGGCTCGCGATTCCTTGGAGCGGGTTGATGACGTCAGCGTTCGAATT
CC
>Microbat_H1_Bidirectional_Promoter (SEQ ID NO: 49)
GGAGAAGGAGGCGTAGACGGCGGATATATAAGGCCCCCTTATGTGTAGTCCTTTTACGGTTAGGGTGACTTCCCA
CAACGCATAGCGACATGCAAATTTGACGGGCGTGCCTCCTCTGTCCCCGGGCAACTTCTCTCCTGGACGCGCGCT
CGCGCTGCGTGTTCCCGCCTTTTGACTCCAGCCGAGCGAATCCTGGGAGAGGGCAGGTGACGTCAACAGTCAGGC
TCG
>Opossum_H1_Bidirectional_Promoter (SEQ ID NO: 50)
GGTGCGGGGCCTCAAAGAGAGCGATATATAACGCTCACAAAACCCGTGCTATTTCTTACAGAGGGTGATATCCCC
ATGATCCCCGGCGGTATGCAAATAGTAGTCGCGTCAGAGCAGAGCGCAGTCAGCCGCTCTCTCCTAGCGCGGGAA
ATCTATTTCTTCTTCAGTCTCGGTAACGAGCGCATGCGCATACTGTAGGTGACCTACGGTTTTGTCAGGAATCGG
TTGGGAGCACC
>Pacific_walrus_H1_Bidirectional_Promoter (SEQ ID NO: 51)
GGGAAACGGTGGCCCCAAAGAGCATTTATAAAGCTCCCTCAACTAAATGCATTTATCAGTTATGGTGACTTCCCA
CAATACATCGCAACATGCAAACATCGCGGGGAGTACCTCCCCTGTCCCTACGTGTCTTCTCAGGACGCACGCACG
CGCGCTGTGTTCCCGCCCTGTGACTCTAGGCGGGCAATTCCTAGAAGACGCTTGCTGACGGGAACGTTCCGGCTC
C
>Pig-tailed_macaque_H1_Bidirectional_Promoter (SEQ ID NO: 52)
GGGGAAAGCCGATCCCAGCCAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Prairie_vole_H1_Bidirectional_Promoter (SEQ ID NO: 53)
GGGAAGGCGGGGCGGCGGCACTAAAAGGCTCCGGAGCGGCCCAGACTTTACAGTTATGGTGGCTTCCCACGAGGC
GCAGCGCCACTCATTTGCATGGACCCGCCCCAGACGGGAAGCCCGCACCGCTCATTTGTGTGGCCCCGCCCCAGA
CGGGAAGCCCGCGCCACTCATTTGC
>Rhesus_H1_Bidirectional_Promoter (SEQ ID NO: 54)
GGGGAAGGGTGGTCCCACACAGAACTTATAAGATTCCCATACTCAAAGACCTTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Ryukyu_mouse_H1_Bidirectional_Promoter (SEQ ID NO: 55)
TGGAGGGTGGAGACCGGCCGCCACTATAAGGCTCGAAAGAGGAATAAATTTTACGTTTAGGGTGATTTCCCACAA
AGCACAGCGCGTAATTTGCATGTGCTCTATCCCAGGCTCCAGTGCCAGACAAGAAGCCCGCGCATCCGGGCAAGG
GATGATGACGTCGTCCTTCAAGAGCG
>Shrew_H1_Bidirectional_Promoter (SEQ ID NO: 56)
GCGTAAGACGCGCCGCATCGCGTACTTATAAGGATCCCCTGGTCAACGATCTTTTACAGTTAGGGTGACTTCCCA
CAGTACACGGCGGTATTCAAATATGAAGGGCGTGTCTAGTCCGGGTCCTGGCTAGGCGCATGTGCAGTGCTGGTT
CCCGCCACTTCCGACGTCTACGTTTAGACTCC
>Shrew_mouse_H1_Bidirectional_Promoter (SEQ ID NO: 57)
TGAAGGCTGTAGACCGGCCGCCACTATAAGGCTCGAAAGAGGAATAAGTTTTTCGCTTACGGTGACTTCCCACAA
AGCACAGCGCGTAATTTGCATGTACTCTATCCCAGGCTTCCTGTTCCAGACTAGAAGCCCGCGCATCCGGGCAAG
GGACGATGACATCATCCCCATCCCTCCAGCGCG
>Sifaka_H1_Bidirectional_Promoter (SEQ ID NO: 58)
GAGGGAAAAGGGTTCTGCACAGAATTTATAAGGCTCCCAAATCTAAAAACATTTCACCATTATGGTGATTTCCCA
CAACACATAGCGACATGCAAATATCTCAGAGCGTACCTCCCCTGTCCTATACGGGCGTCAACTCGCCATGGCGCA
CGCGCGTTGTGTGTTTCCCGCCTGTGACTCTGGGCCCGCGATTCCTCCCAGCGGGTTGAGTACGTCAGCTCCGGT
GCTTC
>Sooty_mangabey_H1_Bidirectional_Promoter (SEQ ID NO: 59)
GGGGAAAGGTGGTCCCACACCGAACTTATAAGACTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGCAGCGGGTTGGTGACGTCAGCGTTCGA
ATTCC
>Squirrel_monkey_H1_Bidirectional_Promoter (SEQ ID NO: 60)
GGGGAAGGGTGGTCCTTCGCAGAACTTATAAGATTCCCAGTCCCGAGGACATTTCTAGATTATGGTGACTTCCCA
GAATACACAGCGACATGCAAATATTGCAGGTCGTGCCTCGCCTGTCCCTCACTGTCGTCTTCCTGCCAGGGCGCA
CGCGCGCTGGGTGTCCCGCCAACTGACACTGGGCTCGCGATTCCTTGGAGCGGGTTGATGACGTCAGCGTTCGAA
TTCC
>Star-nosed_mole_H1_Bidirectional_Promoter (SEQ ID NO: 61)
GCGCAGAGACAAGCTTAGCTAGAATTTATAAGGCGCCCATACTTGCAGACATATATCGGTTAGGGTGACTTCCCA
CAAGCCATAGCGACATGCAAATAGAGAGGGCGGGCTTCCCCTGAGCTTAGGCGTCTTCTTACGAAGTCGCGAGCG
CGTCGCGCGCCTGTTCCCGCCCGGTCACTATTGGCCTGTCACTATTGTCATTCCGCCCTTCCCGGGCGGAGTCTG
GTGACTTTCGGTTCC
>Synthetic-1_H1_Bidirectional_Promoter (SEQ ID NO: 62)
GCAGCGCAGCCCTCTCGCCGCTTATAAAGTGCCGCCCGCACGGCCCTTCTCGCTCACGGCGACTTCCCATAAAGC
ACAGCGCGTAATTTGCATGCGCTCTACCCCAGGCTCCTGTGCTAGACAAGAAGCCCGCGCATCCGGGCAAGGGAT
GATGACGTCAGATCTCC
>Synthetic-2_H1_Bidirectional_Promoter (SEQ ID NO: 63)
GGGGAAAAGTAGTGCCGCTTATAAAGTGCCGCCCGCACGGCCCTTCTCGCTCACGGCGACTTCCCATAAAGCACA
GCGCGTAATTTGCATGCGCTCTACCCCAGGCTCCTGTGCTAGACAAGAAGCCCGCGCATCCCGGACGTCAGATCT
CC
>Tenrec_H1_Bidirectional_Promoter (SEQ ID NO: 64)
AGGTTAAAGCCGCGTCGCCGCGCGCTTATAAGAATCCGGGAACTAACTACATTTCAAGGTCAGGGTGATTACCCA
CCCTGCATAGCGACATGCAAATAGCACGGAACGTCCAGGAGACGTGCCTCTAGGTCTTGGGGAGGGAGGAGTTCG
GCCCAGCGCGCACGCGCACTACGTGTTCCCGCCCGCTGTCTCGGGGCGGGAGATCCCGGGTAGGTGACGTCAGTC
CTCGGCTTC
>Tibetan_antelope_H1_Bidirectional_Promoter (SEQ ID NO: 65)
GGCAAACGACTCCCGCAAACAGCATTTATAATGCGCTCATACATAAAGCCACTTTTCGGTTACGGTGACTTCCCA
CAAGACATTGCGACATGCAAATATTTTAGTGCATCCCGCCCCTGGTAGCTCCACGCTAGGACGCACACGCACTAC
GGTTCCCGCCTTTAGACTGCCGGGGCGATTCCAGGGAGCGGACTGATGACGTCAGCGTTCGGACTCC
>Tree_Shrew_H1_Bidirectional_Promoter (SEQ ID NO: 66)
GGGGGAAGCTGGGTCCACTGAGTTCTTATAAGGTTTCCAGTCCTAGAGCGATTTTACCATTGCGGTGATTTCCCA
GCATCCGTAGCTACATGCAAATAGCGCGGGGCGCGTCTCTCAGGTCCCTCCCCCGTGCCCTCTCACTGTACGTAC
CCGCGTCCTAGGGACGCCGCGCCCGGGGTTCCCGGACGTCAGCGTTCCGACGCA
>Weddell_seal_H1_Bidirectional_Promoter (SEQ ID NO: 67)
GGGGAAGAGTGGCCCCAGAGAGCATTTATAAGGCTCCCGCACCTAAATGCATTTATCAGTTATGGTGACTTCCCA
CAATACATAGCAACATGCAAATATAGCGGGGAGTACCTCCCCTGTCCCTACGTGTCTTCTCAGGACGCACGCACG
CGGGCTGTGTTCCCGCCCTGTGACTCTAAGCGGGCAATTCCTGGAAGAGGCTTGCTGACGGGGACGTTCAGGCTC
C
>White_rhinoceros_H1_Bidirectional_Promoter (SEQ ID NO: 68)
GGAGCAAACATGCGCCAGGCAGCCTTTATAAGACTCACATATCTAAAGACATTTCACAGTTAGGGTGACTTCCCA
CAGGACACAGCGATATGCAAATATCGTGGAGCGTACCTCCCCAGTCTCCGGGCATCTTCTCGCCTACACGCACGC
GCGCCGCGTGTTCCCGCCCTGTGACGCTAGGTGGGCCTTTCATGGGAGAGGGTTGATGACGTCAACATTCGGACT
CC
>White-faced_sapajou_H1_Bidirectional_Promoter (SEQ ID NO: 69)
GGGGAAGGGGTGGCCTACGCAGAACTTATAAGATTCCCACACCTAAAGACATTTAACGATTATGGTGACTTCCCA
GAATACACAGCGACATGCAAATATTGCAGGTCGTACCTCGCCTGTCCCCCACAGTCGTCTTCCTGCCAGGGCGCA
CGCGCGCTGGGTGTCCCGCCAACTGACAGTGGACTCGCGATTCCTTGGAGCGGGTTGATGACGTCAAAGTTCGAA
TGCC
>Alpaca_H1_Bidirectional_Promoter (SEQ ID NO: 70)
GGGAAAGGGTGGGCTCACGCAGCCTTTATAAGACTCCCAAACTTAAAGACATTTCTCGGTTATGGCGACTTCCCA
CAAGACATAGCGACATGCAAATACTGCAGACCTGTGGCGCCGACCCGGTCCTGTGCAGCCATCTTTACGGCTGGG
ACGCACGCGCGCTGCGTGTTCCCGCCCTGTGACTGCGCCGGCGATTACTGGGAGAGGATTGATGACGTCAACGTT
CGGGTTCC
>Armadillo_H1_Bidirectional_Promoter (SEQ ID NO: 71)
AAAGCGATAGTTTTTTAAACTGGACTTATAAGGCACCCATATCTACGTATATTTCATGGTTAGGGTGATTTCCCA
CAACACATAGCGAAATGCAAATATGTGGAGCGGGCGCTGAGGCGTGGTCGGGCGCAAGCGCGCTGCGACTTCCCG
CCTTTCGGCCCTAGGCCCCAGATTCCTGGGAGCTGGATGATGACGTTGACGTTCGGATACC
>Baboon_H1_Bidirectional_Promoter (SEQ ID NO: 72)
GGGGAAAGGTGGTACCATACAGAACTTATAAGATTCCCATACTCAAAGACATTTCACGATTATGGTGACTTCCCA
GAAGACACAGCGACATGCAAATATTGTAGGGCGTCACACCCCCTGTCCCTCACAGTCATCTTCCTGCCAGGGCGC
ACGCGCGCTGGGTGTTCCCGCGTAGTGACACTGGGCCCGCGATTCCTTGGAGCGGGTTGATGACGTCAGCGTTCG
AATTCC
>Bottlenose_dolphin_H1_Bidirectional_Promoter (SEQ ID NO: 73)
GCCGAAAACCAGGCTCAAGCCACATTTATAAGGCTCCCAAATCTAAGTACATTTGTCGGTTATGGTGACTTCCCG
CACCACATTGCGACATGCAAATACTGCGGAGCGTCCCTCCCCTGGCAACTCCTCGCTGGGACGCACGCGCGCTAC
GTGCTCCCGCCTTTTGACTGCGCCGGCGATACTTGGGAGAGGGTTGATGACGTCAGCGTTCTGGCTCC
>Bushbaby_H1_Bidirectional_Promoter (SEQ ID NO: 74)
GCCTAAAAGGGCGCTTGCACAGAATTTATAAGGTTCCCAAACAGAGACACATTTCATTATTATGGTGACTTCCCA
CAATGCACAGCGCCATGCAAATATGCTAGGACCTGCCTCCCCACACCCGCTACCTTAAGGTCGTCAACTAACCAG
TGCGCGCGCGCACTGCGCGTTTCCCGCCGGTGACTCAATGCCCGCGTTTGGTGGGAGCTAGTTGGTGACCTCAGT
TCTGGAGGCTC
>Cat_H1_Bidirectional_Promoter (SEQ ID NO: 75)
GGGAAAGGGTGGCCCCGCCGAGCATTTATAAGACTCCCATACCTAAAGACATTTCTCAGTTATGGTGATTTCCCA
CAACACACAGCAACATGCAAATATCGAGGGGTGTACCGCCCCTGTCCTTAGACGTCTTCTCTCCAGGACGCACGC
GCGCTGTATTCCCGCCTTGTGACTCTAGGCGGGCGATTCCTGGGAGAGGGTTGATGACGTCCAAGTTCTGGCTTC
>Chimp_H1_Bidirectional_Promoter (SEQ ID NO: 76)
GGGAAAGGGTGGTGCCACACAGAACTTATAAGACTCCCATATGCAAAGACATTTCTCGTTTATGGTGATTTCCCA
GAACACATAGCGACATGCAAATATTGCAGGGCGCCACTCCCCTGTCCCTCACTGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Cow_H1_Bidirectional_Promoter (SEQ ID NO: 77)
GGCAAACACCGCACGCAAATAGCACTTATAATGTGCTCATACCTAGAGCCACTTTTCGGTTACGGTGACTTCTCA
AAAAGACAGTGGAACATGCAAATATTACAGTGCGTCCCGCCCCTGGTAGGTCTACGCTAGGACGCACGCGCACTA
CGGTTCCCGCCTATAGACTGCGCTGGCGATTCCTGGGAGCGGACTGATGACGTCAGCGTTCGGGATCC
>Crab-eating_macaque_H1_Bidirectional_Promoter (SEQ ID NO: 78)
GGGGAAGGGTGGTCCCACACAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Dog_H1_Bidirectional_Promoter (SEQ ID NO: 79)
GCAGCGCAGCCCTCTCGCCGCTTATAAAGTGCCGCCCGCACGGCCCTTCTCGCTCACGGCGACTTCCCATAACAC
ACAGCAGCATGCAAATACCGCGGGGAGCCCCGCCCCGCCCCGGCCCCCGCACCGCCTCGGGACGCATGCGCCGGC
TCTCCGTTCCCGCCTTGGGCCGGCGGCGGGCGGGCGGGCGAGCGGGCGGGAGCGGCTCCGGCGAGCGGGCGCC
>Elephant_H1_Bidirectional_Promoter (SEQ ID NO: 80)
GGGATAGGAACAAATTCGTCAGGATTTATAAGACTCTCAGAGCTGTAGACATTTCACAGTTAGGGCGATGTCCCA
CAATACATAGCAACATGCAAATACATGAGCCTTCTAGGAGGCCAGCCTCCCCGTCCGCGTGGTCATCTTCTCGCT
AGGGCGCACGCCCGCTGCGTGTTCCCGCTCTGTGACCAGGCAGGCGATTCCTGAGAACCGCTTGGTGACGTCAGT
GTTCTGGCTCC
>European_Hedgehog_H1_Bidirectional_Promoter (SEQ ID NO: 81)
GCCTAAACCGGCTCTTTCGACAGACTTATAAGGACCTCTTATCTTAGGACATTTTTTTGTTAGGGTAACTTCCCA
CGATGCATAGCGATATGTAAATATGGCGCCGCGAGTCTCTCCTAGGCGTCTCCCCAGGACGCAGGCGCACTGCTT
GTTCCCGCGTTAACATTGCTGATTCTGGGAGACTGCTGATGACGTCAGCGTCCAGTCTAC
>Ferret_H1_Bidirectional_Promoter (SEQ ID NO: 82)
GGGAAAGGGTGGACCCACCGAGCATTTATAAGGCTCCCGCATCTAAAGACATTTTACAGTTATGGTGACTTCCCA
CAACGCGTAGCAACATGCAAATATCGTGGAGAGTACCGCCCCTGTCCCCACGCGTCTTCTCAGCACGCACGCACG
CGCGCTGTGTTCCCGCCCTGTGACTCCAGGCGGGTATTTCCAGGGGCGGGTTTGCTGACAGGAACGTTCAGGCTT
C
>Gorilla_H1_Bidirectional_Promoter (SEQ ID NO: 83)
GGGAAAGGGTGGTCCCACACAGAACTTATAAGACTCCCATATCCAAAGACATTTCACGGTTATGGTGATTTCCCA
GAACACATAGCGACATGTAAATATTGCAGGGCGCCACTCCCCAGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Green_monkey_H1_Bidirectional_Promoter (SEQ ID NO: 84)
GGGGAAGGGTGGTCCCTTACAGAACTTATAAGATTCCCAAACTCAAAGACATTTCACGTTTATGGTGACTTCCCA
GAAGACATAGCGACATGCAAATATTGCAGGGCGTCACACCCCTCTCCCTCACAGTCATCTTCCTGCCAGGGCGCA
CGCGCGCTGGGTGTTCTCGCGTAGTGACACTGGGCCCGCGATTCCTTGGAGCGGGTTGATGACGTCAGCGTTCGA
ATTCC
>Guinea_pig_H1_Bidirectional_Promoter (SEQ ID NO: 85)
GAGAAAGAAAGGCTCAAACCTAGCCTTATAAGGCTCCCAAATGTCGGTATATTTTTTGGTTATGGTGACTTCCCA
CAATGCATAGCGATATGTAGATATAGGAGTACCTCCCACTTCTGGTCCGTCAGCTCTTTTCTAGGACGCGCGCGC
TGCAGGTTTCCAGCCTGTGATTGGGCCAGCAATTCCGGGAATGAATTGATGACGTCAGCGTTTGAATTCC
>Horse_H1_Bidirectional_Promoter (SEQ ID NO: 86)
GGGGGAAAACAGCCCATGGCTGCATTTATAAGACTCACAGATCTAAAGCCATTTCACGAATAGGGTGACTTCCCA
CAATACACAGCGACATGCAAACATAGCGGGGCGTGCCTTTCCTGTACCTGGGCATCTCTCCTGGACGCACGCGCG
CCGGGTGTTCCCGCGCTGTGACTCTAGGCAAGCGCTTCCTGGGAGAGAGTTGATGACGGCAGCATTCGGGCTCC
>Human_H1_Bidirectional_Promoter (SEQ ID NO: 87)
GGGAAAAAGTGGTCTCATACAGAACTTATAAGATTCCCAAATCCAAAGACATTTCACGTTTATGGTGATTTCCCA
GAACACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Kangaroo_Rat_Bidirectional_Promoter (SEQ ID NO: 88)
AGGAAAGACTTCGCTGAGGCAGACTTTATAAGGCTCCCGCGCAGAAAGAAACTTTATAGTTATGGTGATTTCCCA
CAAGCCACTGCGTCATGCAAATAAAGCAGGGTACGGCTTCCATGTACCTTAAGGTTTTTTTCTAGGCCGCGTACG
CTCTGCGTATTCAGCCACGTGACCCTGAGCCAGTGGTTGTTGGGAGCACGTTGTGGACCTCTGCGTTTGGATTCC
>Large_flying_fox_H1_Bidirectional_Promoter (SEQ ID NO: 89)
GCGAGAAAAATTCTTCACGCAGAATATATAAGGATCCCATATCTGAAGACATTTTACGATTACGGCGATTTCCCA
CAACACATAGCGACATGTAAATGTAGTGGGGCATGCCTCCCCTGTCCCTGGGCAGCTTCTCGCCAGAACGCACGC
GCGGTGCGTGTTCCCGCCTTGTGACTAAGTTGGCGAGTCAGGGAGGAGATTGATGATGTCATCATCGTCAGCTCA
CCCGCTCC
>Little_Brown_Bat_H1_Bidirectional_Promoter (SEQ ID NO: 90)
GGGAGAAGGAGGCGTAGAGGATATATAAGGCCCCCTTATGTGTAGTCCTTTTACGGTTAGGGTGACTTCCCACAA
CGCATAGCGACATGCAAATTTGACGGGCGTGCCTCCTCTGTCCCTGCGGGCAACTTCTCTCCTGGACGCGCGCGC
GCTGCGTGTTCCCGCCTTTTGACTCCAGCCGAGCGAATCCTGGGAGAGGGCAGGTGACGTCAACAGTCAGGCTCG
>Marmoset_H1_Bidirectional_Promoter (SEQ ID NO: 91)
GAGGAAAAGTAGTCCCACAGACAACTTATAAGATTCCCATACCCTAAGACATTTCACGATTATGGTGACTTCCCA
GAAGACACAGCGACATGCAAATATTGCAGGTCGTGTTTCGCCTGTCCCTCACAGTCGTCTTCCTGCCAGGGCGCA
CGCGCGCTGGGTTTCCCGCCAACTGACGCTGGGCTCGCGATTCCTTGGAGCGGGTTGATGACGTCAGCGTTTGAA
TTCC
>Mouse_H1-1_Bidirectional_Promoter (SEQ ID NO: 92)
TTCAGGATGTAGACCGGCCGCCACTATAAGGCTCGAAAGAGGAATAAATTTTTCGTTTAGGGTGATTTCCCACAA
AGCACAGCGCGTAATTTGCATGCGCTCTACCCCAGGCTCCTGTGCTAGACAAGAAGCCCGCGCATCCGGGCAAGG
GATGATGACGTCGTCCTTCAAGAGCG
>Mouse_H1-2_Bidirectional_Promoter (SEQ ID NO: 93)
TTCAGGATGTAGACCGGCCGCCACTATAAGGCTCGAAAGAGGAATAAATTTTTCGTTTAGGGTGATTTCCCACAA
AGCACAGCGCGTAATTGCATGCGCTCTACCCCAGGCTCCTGTGCTAGACAAGAAGCCCGCGCATCCGGGCAAGGG
ATGATGACGTCGTCCTTCAAGAGCG
>Northern_Treeshrew_H1_Bidirectional_Promoter (SEQ ID NO: 94)
GGGGGAAGCTGGGTCCACTGAGTTCTTATAAGGTTTCCAGTCCTAGAGCGATTTTACCATTGCGGTGATTTCCCA
GCATCCGTAGCTACATGCAAATAGCGCGGGGCGCGTCTCTCAGGTCCCTCCCCGCCCTCTCACTGTACGTACCCG
CGTCCTAGGGACGCCGCGCCCGGGGTTCCCGGACGTCAGCGTTCCGACGCA
>Orangutan_H1_Bidirectional_Promoter (SEQ ID NO: 95)
GAGAAAGGGTGGTCCCGTCCAGAACTTATAAGATTCCCATACCCAAAGACATTTCACGTTTATGGTGACTTCCCA
GAATGCATAGCGACATGCAAATATTGCAGGGCGTCACTCCCCTGTCCCTCACAGCCATCTTCCTGCCAGGGCGCC
CGCGCGCTGGTGTTCCCGCCTAGTGACACTGGGCCCACGATTCCTTGGAGCGGGTTGATGACGTCAGCGCTCGTA
TTCC
>Panda_H1_Bidirectional_Promoter (SEQ ID NO: 96)
AGGGAAAGCCGCGCCTGGGGCGGATTTATAAGGCTTCCATATCTAAAGGCATTTCACAGTCATGGTGACTTCCCA
CAATACATAGCAACATGCAAATATCGCGGGGAGAACCTCCCCTGTCCCTTGTACGCGGCTTCTAAAGACGCACGC
ACGCGCTCTGTGTTCCCGCCCTGTGACTCTAGGCGGGCAATTCCTGGGACAGTGTTCTGACGGGAACGTTCAGGC
TCC
>Pig_H1_Bidirectional_Promoter (SEQ ID NO: 97)
GGAAAACTGCTTCTGTGAGCACTTATAAAACTCCCATAAGTAGAGAGATTTCATAGTTATGGTGATTTCCCATAA
GACATTGCGACATGCAAATATTGTGGCGCGTTCGTCCCCGTCCGGTGCAGGCAGCTTCGCTCCAGGACGCACGCG
CAATACATGTTCCCGCCTTGAGACTGCGCCGGCAGATTCCTAGGAAGTGGTTGATGACGTCGATGTTAGGGATCC
>Pika_H1_Bidirectional_Promoter (SEQ ID NO: 98)
GGGGGAAGCTGGGCTCGATCAGCCTTTATAAAGCTCCAAAAACTCAAGACATTTTTCCGTTACGGTGGCTTCCCA
CAGTACACAGCGACATGCAAATAGGCGGACCGCTTCCCGCTCCGGCGCAGGCGCGCGGGCGCTGTCTCCCCTGGA
CGCGCGCTCGCGGTTCCCGGGAGCTGGCTGATGACGTTCGGTCTCC
>Rabbit_H1_Bidirectional_Promoter (SEQ ID NO: 99)
GGGGAGAGGTGGATCCGAACAGACTTTATAAAGCTCCGAAAGCCCAAGGCATCTTTCCCTTACGGTAGCTTCCCA
CAAGACATAGCGACATGCAAATTTCAGACGCGCTTCTCGCCACAGCGCAAGCGCGCTGTGTGCTGACGCGGGAAC
GGGCCAGGGCGCGGTTCCCGGGAGCGGGTTGATGACGTTAGATCTCC
>Rat_H1_Bidirectional_Promoter (SEQ ID NO: 100)
AGGAGTGTGAAGACCTGCCGCCATAATAAGACTCCAAAAGACAGTGAATTTAACACTTACGGTGACTTCCCACAA
AGCACAGCGTGTAATTTGCATGCGCTCTAGCCCAGGCTCCAGCTCCGGACCAGAAGCCCGCGCATCCCGGCAAAG
GGTGATGACGTCGTCCTTCAAGCGCT
>Rock_Hyax_Bidirectional_Promoter (SEQ ID NO: 101)
AGGGTAAATCGGCGCTGCTCAGCATTTAAAAGAATCCCAAATGTGTCGCCATTTTACGCTTAGGGTGATATCCCA
CAAGACACAGCGACATGCAAATATCGTGAGTCTCTGTTTCCCTGTCCACGAGGGCGTCCTCTCGCTGGGGCGCAC
GCGCGGTGTGTGTGCCCCCGTTGTGTGTTCCCGCGATTCCAAAGAACTGGTTGATAACGTTAGACTTCCGGCTGC
>Sheep_H1_Bidirectional_Promoter (SEQ ID NO: 102)
GGCGAACAATGCGCGCAAACAGCATTTATAATGAGCTCATACCTAAAGCCACTTTACGGTTACGGTGACTTCCCA
CAAGACATTGCGGCATGCAAATATTTTAGTGCGTCCCGCCCCTGGTAGTTCCACGCTAGGACGCACACGCACTAC
GGTTCCCGCCTTTAGACTGCGCTGGCGATTCCAGGAGCGGACTGATGACGTCAGCGTTGGGGCTCC
>Squirrel_H1_Bidirectional_Promoter (SEQ ID NO: 103)
GAAAGGGACTCCGCACAAGCAGAGTTTATAAGGCTCCCATCTGTACAGCCATTTCTCGGTCATGGTAACTACCCA
CAACACACAGCGATATGCAAATATAGCAGAGCGTGTCTTCCCGCGCGCGCCTGGTCGTCTCGGCGCCGGCGCCGG
AACTGTGACAGAGACCCTGCGATTCCTGGGAGCTGGCTGATGACATCAGTGTCTAACCTCC
>Tarsier_H1_Bidirectional_Promoter (SEQ ID NO: 104)
GCGAGAGGGTGGGTCCACACAGAGCTTATAAGGCTTCACAAGTAAAGATATTTCACGGTGACGGTGACTTCCCAC
AATACACTGCGACATGCAAATATAGCCGGGCGTGCCTCCCCGATCCCGGAAGAGCGACTCCTAGCCAGTGCGCAC
GCGCGCTGCGTGTTCGCGTCCTAGGTCGCTGGGCCCGCGGTTCCTGGGAGCGGGTGGTGACGTCAGCGGCCCAGC
TTC
>Two-Toed_Sloth_H1_Bidirectional_Promoter (SEQ ID NO: 105)
AGAAAAAAATAGTTTATGCTGGATTTATAAGATTCCCAAATCTAAAGCCATTTCACAGTTACGGTGATTCCCCAC
TACACACGGCGATATGCAAATATAGCGGAAGTGTTCCTGAGGCGTGGTAAAGCGCGCGCGCGCTGAGAGTTCCCG
CCCTGTGGTGCTGGGCTGGAGATGCCTGAGAACTGGCTGATGACGGCAACGTTCGGGCTCC
>White_cheeked_gibbon_H1_Bidirectional_Promoter (SEQ ID NO: 106)
GGGGAAAAGTAGTAGACCTTATAAGATTCCCAAACCCAAAGACATTTCTCGTTTATGGTGACTTCCCAGAAGACA
TAGCGACATGCAAATATTGCAGGGCGCCACTCCCCTGTCCCTCACAGCCATCTTCCTGCCAGGGCGCACGCGCGC
TGGGTGTTCCCGCCTAGTGACACTCGGCCCGCGATTCCTTGGAGCGGGTTGATGACGTCAGCGTTCGAATTCC
>GAR1-1_Bidirectional_Promoter_Homo_sapiens (SEQ ID NO: 107)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTCAG
>GAR1-2_Bidirectional_Promoter_Homo_sapiens (SEQ ID NO: 108)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTCAGGCAAGTTGGCCTCTC
TGTTGTAAATTAGTGGTTAAGGTTATCTATTATTGCCACTTTTCCAGCGCTAAAGGCTGTTTTGGAACCAGTGTT
GCTTGTTCCGCGGGTGATTGGCTTTTTTTTTTGGCAAACCAGTTATTCAAGTTTCTGGTCTTTAAAAAACTCTGT
GGCGGTACGGTAACCGAGGAGGTTCCAGCGCGGCGGAAGTACCCCGCGGGTGGGTGTGTGCGCAAGGCCAGGGCC
AGAGGGGCACGTGGCGCCG
>macaca_mulatta/1-143_Gar-1 (SEQ ID NO: 109)
CCCACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGCTATTATACGTCACTTCCACGGCGCGGCGTTAG
>ancestral_sequences9/1-143_Gar-1 (SEQ ID NO: 110)
CCTACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGCTATTATACGTCACTTCCACGGCGCGGCGTTAG
>papio_anubis/1-143_Gar-1 (SEQ ID NO: 111)
CCTACCCAGCCTCCGGAAGAGAAGGCGGATCCCTAACGCCAGCTATCTCCAAGAGCAACATTGCCGCACCACTTC
CGGGACGTCGTGCTGCGAAGGACGCAGTTATTATACGTCACTTCCACGGCGCGGCGTTAG
>ancestral_sequences10/1-143_Gar-1 (SEQ ID NO: 112)
CCTACCCAGCCTCCGGAAGAGAAGGCGGATCCCTAACGCCAGCTATCTCCAAGAGCAACATTGCCGCACCACTTC
CGGGACGTCGTGCTGCGAAGGACGCAGCTATTATACGTCACTTCCACGGCGCGGCGTTAG
>ancestral_sequences11/1-143_Gar-1 (SEQ ID NO: 113)
CCTGCCCCGCCTCTGGGAGAGAAGGCGGATCCCTAACGCCAGCTATCTCCAAGAGCAACATTGCCGCAGCACTTC
CGGGACGTCGTGCTGGGACGCCGCTATTATACGTCACTTCCACGGCTCCGCGTTAG
>callithrix_jacchus/1-143_Gar-1 (SEQ ID NO: 114)
CCCGCCCCGCCCCCGGTAGAGAGGGCGGATCTCTAACGCCAACTATCTCCAAGAGCAACATTGCCGCAGCACTTC
CGGGATGTCGTGCTGCGAAGGACGCCGCTATTGTACGTCACTTCCGCTTCTCCACTCTAG
>pan_paniscus/1-191_Gar-1 (SEQ ID NO: 115)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACAGCTCAGCGTCAG
>pan_troglodytes/1-191_Gar-1 (SEQ ID NO: 116)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCCGCGTCAG
>pongo_abelii/1-191_Gar-1 (SEQ ID NO: 117)
CCTGCCCCGCCTCTGGGAGAGAAGGCGGATCCCTAACGCCAGCTATCTCCAAGAGCAACGTTGCCACAGCACTTC
CGGGACGTCGTGCTGCAAAAGACGCCGCTGTTATACGTCACTTCCACGGCTCAGCGTTAG
>nomascus_leucogenys/1-191_Gar-1 (SEQ ID NO: 118)
CCTGCCCCGCCTCTGGGAGAGAAGGCGGATCCCTAACTCCAGCTATCTCCAAGAGCAACATTGCCGCAGCACTTC
CGGGACGTAGTGCTGCAAAGGACGCCGCTGTTATACGTCACTTCCACGTCTCAGCGTTAG
>chlorocebus_sabaeus/1-191_Gar-1 (SEQ ID NO: 119)
CCTACCCCACCTCTGGAAGGGAAGGCGGATCCCTAACGCCAGCTATCTCCAAGAGCAACATTGCCGCAGCACTTC
CGGGACGTCGTGCTGGGACGCAGCTATTATACGTCACTTCCACGGCGCCGCGTTAG
>macaca_nemestrina/1-143_Gar-1 (SEQ ID NO: 110)
CCCACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGATATTATACGTCACTTCCACGGCGCGGCGTTAG
>colobus_angolensis_palliatus/1-143_Gar-1 (SEQ ID NO: 111)
CCTGCCCCGCCTCTGGGAGAGAAGGCGGATCCCTAACGCCAGCTATCTCCAAGAGCGACATTGCCTCAGCACTTC
CGGGACGTCGTACTGCAAAGGACGCAGTTATTATACGTCACTTCCACGGCGCCGCGTTAG
>piliocolobus_tephrosceles/1-143_Gar-1 (SEQ ID NO: 112)
CCTGCTCCGCCTCTGGGAGAGAAGGCGGATCCTTAACGCCAGCTATCTCCTAGAGCAACATTGCCTCAGCACTTC
CGGGACGTCGAGCTGCAAAGGACGCAGTTATTATACGTCACTTCCAGGGCGCCGCGTTAG
>rhinopithecus_bieti/1-143_Gar-1 (SEQ ID NO: 113)
CCTGCCCCGCCTCTGGGAGAGAAGGCGGATCCCTAACGCCAGCTATCTCCAAGAGCGACATTGCCTCAGCACTTC
CGGGACGTAGTGCTGCAAAGGACGCAGTTATTATACGTCACTTCCACGGCGCCGCGTTAG
>aotus_nancymaae/1-143_Gar-1 (SEQ ID NO: 114)
CCCGCCCCGCCCCTGGGACAGAGGGCGGATCTCTAACGCCAACTATCTCCAAGAGCAACATTGCCGCAGCACTTC
CGGGACGTCGTGCTGCAAAGGACGCCGCTATTATACGTCACTTCCGCGGCTCCAG
>cebus_capucinus/1-143_Gar-1 (SEQ ID NO: 115)
CCCGCCCCGCCCCTGGGAGAGAGGGCGGATCTCTAACGCCAACTGTCTCCAAGAGCAACATTGCCGCAGCACTTC
CGGGACGTCGTCCTGCAAAGGACGCCGCTATTATACGTCACTTCTGCTGCTCACTGTAG
>saimiri_boliviensis_boliviensis/1-143_Gar-1 (SEQ ID NO: 116)
CCCGCCCCGCCCCTGGGAGAGAGGGCGGATCTCTAACGCCAACTATCTCCAAGAGCAACATTTCAGCAGCACTTC
CAGGACGTCGCCCTGCAAAGGACGCCGCTATTATACGTCACTTCCGCTGCTCCACTCTGG
>carlito_syrichta/1-143_Gar-1 (SEQ ID NO: 117)
CCTGCCCCGCCTCTAGAGAAGGGGACGGATTCGTAATGCCCGGCAATCGCGCAGCCGCATTTCCGGGACGTCACG
AGGAAAGGGCGCCGAATTGTATGTCATTTCCGCTTTTCATGGCTGG
>otolemur_garnettii/1-143_Gar-1 (SEQ ID NO: 118)
CTCGGCCAGTCTCAGGCAGAAAGGGCGGAAACCGGACCCCAGCGCAATGTCACGGCAGCACTTCCGGTATGCTCC
GTTGCAAAAGACGCTGCTATTGTACGTCACTTCCGCCACCCGGCTGG
>prolemur_simus/1-143_Gar-1 (SEQ ID NO: 119)
CCCGCCCCGCCTCTCGGAGACGGGGCGCGTCCCTCCCGCCGCCGTCTCCCGGGGCAACATGGCGGCAGCACTTCC
GGGGCGCCGGTGGCGAAAGGCGCCGCTATTATACGTCACTTCCGCCGCCCGGCGCGAG
>propithecus_coquereli/1-143_Gar-1 (SEQ ID NO: 120)
CTGGCCCAGCCTCTTATGGCGGGGGCGGACCCCTTACGCCAGCTATCGCCCAGGGCAATATGGCGACATCACTTC
CGGTATGTCAGGTTGTGAAAGGCGCCGCTATTGTACGTCACTTCCGCTGCCCAGCGCGGG
>castor_canadensis/1-143_Gar-1 (SEQ ID NO: 121)
CACAACTCGCCTCTGAGAGAGGAGGCGGATCCCTAACGCCTGCTATCTCCAAGGGCAACACTGCGGCATACTTCC
GGAACGTCAGCTCGATGGGACGCGGTTATTTTACGTCACGTCCGCTACTCTCACTCGG
>calJac3_Gar-1 (SEQ ID NO: 122)
CCCGCCCCGCCCCCGGTAGAGAGGGCGGATCTCTAACGCCAACTATCTCCAAGAGCAACATTGCCGCAGCACTTC
CGGGATGTCGTGCTGCGAAGGACGCCGCTATTGTACGTCACTTCCGCTGCTCCACTCTAG
>otoGar3_Gar-1 (SEQ ID NO: 123)
CTCGGCGTCAGTCTCAGGCAGAAAGGGCGGAAACCGGACCCCAGCGCAATGTCACGGCAGCACTTCCGGTTATGC
TCCGTTGCAAAAGACGCTGCTATTGTACGTCACTTCCGCCACCCGGCTGG
>speTri2_Gar-1 (SEQ ID NO: 124)
ACGCCCGACGGGAGAGGAGGCGGGTCCCTAACTCCGCTATCTCCTAGGGCAACTCGACGGCAATACTTCCGGTAA
CGTCCTGACGTAATGGATGCCGTTTCGCTTTACTTCCGCTTTCTCTTG
>micOch1_Gar-1 (SEQ ID NO: 125)
ACGCCCCGCTGTCTCCAAGGGCAACGAGAGACCTCACTTCCTGAAACGTCTCGTACAGAGGGCGCTGCTATTCTA
TGTCACTTCCGCTCCCCGGG
>criGril_Gar-1 (SEQ ID NO: 126)
AAGCCTCACTATAGGACGGAAGGATCCAGACTCCCGCTGTCTCCAAGGGCAACGCGCTACCACACTTCCGGAAAC
GTCGCGTACGGAGGGCACTGCTATTTTGCGTCACTTCCGCTACCCCGGC
>mesAurl_Gar-1 (SEQ ID NO: 127)
ACGCCTCACTCTAGAACGGAAGACTCCAGACGCCCGCCGTCTCCAAGGGCAACGCGCGACCACACTTCCGGAAAC
GGCGCGTACGGAGGGCGCTTCTATTTTGCGTCACTTCCTCTCCTCCAGG
>mm10_Gar-1 (SEQ ID NO: 128)
ACGCCTCACTGTAGCACGGAAGGACTCAAACAACTCCGTTTCCAAGGGCAACGCGCCGCCACACTTCCGGAAACG
TCGCGTACGGAGGGCGCTGCGATTTTGCGTCACTTCCGCCACCTCTAG
>microcebus_murinus/1-191_Gar-1 (SEQ ID NO: 129)
GCGGCGCCAGCCTCTGGGAGAGGGGGCGGACCCTTACGCCAGCTGTCTCCAAGGGCAATATAGCGGCAGCACTTC
CGGTAGCGACAGGTTGTGAAAGACGCCGCTGTTGTACGTCACTTCCGCTGCCCAGAGCGAG
>cavia_porcellus/1-191_Gar-1 (SEQ ID NO: 130)
CGAGTTGCTTCGGGCCTACTAACATCATGCGGCGTTTCTGGAAGAGGAGCCCGCTTCCGGACGCCCGCCGTCTCC
AGGGGCAACACTTCCGTGAACGTCATGTGTAAGGGACGGGTTACGTCACTTCCTGTGCTCCTTGGCT
>marmota_marmota_marmota/1-191_Gar-1 (SEQ ID NO: 131)
CGCCCGACTTCTGGCAAGAGGAGGCGGGTCCCTAACTCCGCTATCTCCTAGGGCAACACGACGGCAATACTTCCG
GTAACGTCCTGACGTAATGGTTGCCGTTTCGCTTTACTTCCGCTTTCTCTTGCTAA
>sciurus_vulgaris/1-191_Gar-1 (SEQ ID NO: 132)
CGCCCAGCCTCCGGGAAGAGGAAGCAGCTCCCGAATACCGGCTATCTCCAAGGGCAACACCACTGCAATGCTTCC
GGAAACGTCATGGCGTAATGGACGCCGTTACAACTTCACTTCCGCTTCTCTCGCTAC
>mus_caroli/1-191_Gar-1 (SEQ ID NO: 133)
CACGCCTCAACAGCTGTTAGCACGGAAGGACCCAAACAACCCCGTCTCCAAGGGCAATGCGCCGCCACACTTCCG
GAAACGTCGCGTACGGAGGGCGCTGCGATTTTGCGTCACTTCCGCCACCTCTAGCG
>mus_musculus/1-191_Gar-1 (SEQ ID NO: 134)
CACGCCTCACCAGCTGTTAGCACGGAAGGACTCAAACAACTCCGTTTCCAAGGGCAACGCGCCGCCACACTTCCG
GAAACGTCGCGTACGGAGGGCGCTGCGATTTTGCGTCACTTCCGCCACCTCTAGCG
>mus_spretus/1-191_Gar-1 (SEQ ID NO: 135)
CACGCCTCACCAGCTGTTAGCACGGAAGGACTCAAACAACTCCGTCTCCAAGGGCAACGCGCCGCCACACTTCCG
GAAACGTCGCGTACGGAGGGCGCTGCGATTTTGCGTCACTTCCGCCACCTCTAGCG
>mus_pahari/1-191_Gar-1 (SEQ ID NO: 136)
CCCAAACAACCCCGTCTCCAAGGGCAACGCGTCGCCACACTTCCGGAAACGTCGCGTACGGAGGGCGCTGCGATT
TCGCGTCACTTCCGCCACCTCTAGCG
>oryctolagus_cuniculus/1-191_Gar-1 (SEQ ID NO: 137)
CAACCGTAAACCCCAGCAGAAAGAACAGGCGGAGCCCTAACACCAACCTTCTCCCGGAGACACGCCCCCTGCTGC
ACTTCCGGAATGTTCTGGGGCAAAGGGCGCCGCTATTATACGTCACTTCCGCCGCGGTTCTTTCG
>balaenoptera_musculus/1-191_Gar-1 (SEQ ID NO: 138)
CAGCCGAGCCGCTGGGAGAGGGGCGGTCCCTGACGCCAGCCATCGCCAAGGGCAACGCCGCGGGGCGGCACTTCC
TGCAACGTCACGCTGCCAAGGACGCCGCTATTGTACGTCACTTCCTCCGCTCTCCGTAG
>delphinapterus_leucas/1-191_Gar-1 (SEQ ID NO: 139)
CAAGCCGATCCGCTGGGAGAGGGGCGGTCCCTGACGCCAGCCATCGCCAAGGGCAACGCCGCGGGGAGGCACTTC
CTGCAACGTCACGCTGCAAAGGACGCCGCTATTGTACGTCACTTCCTCCACTTCCCGGAG
>monodon_monoceros/1-191_Gar-1 (SEQ ID NO: 140)
CAAGCCGATCCGCTGGGAGAGGGGCGGTCCCTGACGCCAGCCATCGCCAGGGGCAACGCCGCGGGGCGGCACTTC
CTGCAACGTCACGCTGCAAAGGACGCCGCTATTGTACGTCACTTCCTCCACTTCCCGGAA
>phocoena_sinus/1-191_Gar-1 (SEQ ID NO: 141)
CAAGCCGATCCGCTGGGAGAGGCGCGGTCCCTGACGCCAGCCATCGCCAAGGGCAACGCCGCGGGGCGGCACTTC
CTGCAACGTCACGCTGCAAAGGACGCCGCTATTGTACGTCACTTCCTCCGCTCTCCGTAG
>physeter_catodon/1-191_Gar-1 (SEQ ID NO: 142)
CAAACCGAGCCGCTACTAGAGGGGCGGTCCCTCACGCCAGCCATCGCCAAGGGCAACGCCGCGGGGCGGCACTTC
CTGCAACGTCACGGCGCAAAGGACGCCGCTATTGTACGTCACTTCCTCCGCTCTCCGTAG
>bos_grunniens/1-191_Gar-1 (SEQ ID NO: 143)
CTTGCTGGGCCGCGGGGAGAGGGGCGGACCCTGACGCCAGTCATCGCCAAGGGCAACGCCGCAGAGCGGAACTTC
CTGCAACGTCATGCTTCCAAGGACGCCGATATTGTGTGTCACTTCCTCTGCTCGCCGTAG
>capra_hircus/1-191_Gar-1 (SEQ ID NO: 144)
CTTGCCCGGCCGCGGGGAGAGGGGCGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTCGCCGTAG
>ovis_aries/1-191_Gar-1 (SEQ ID NO: 145)
CTTCCCGGGCCGCGGGGAGAGGGGCGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTGGCAGTAG
>ovis_aries_rambouillet/1-191_Gar-1 (SEQ ID NO: 146)
CTTGCCGGGCCGCGGGGAGAGGGGCGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTGGCAGTAG
>cervus_hanglu_yarkandensis/1-191_Gar-1 (SEQ ID NO: 147)
CTGGCCGGGCGGCGGGCAGAGGGGCGGGCCCTGACGCCAGTCGTCGCCAAGGGCAACGCCGCAGAGCGGAACTTC
CTGCAACGTCATGCTTCAGAGGACGCCGATATTGTATGTCACTTCCTCTGCTCGCCATAG
>catagonus_wagneri/1-191_Gar-1 (SEQ ID NO: 148)
CCCGCCTGGCCACTGGGAGAGGGGCAGTCCCTGACGCCAGTCATCGCCAAAGGGCAACCCCGCGGGGTTCCTGCA
AGCAACGTCATGCCGCAAAGGACGCCGCTATTTTACGTCACTTCCTCTGCTCCCGTTAG
>sus_scrofa/1-191_Gar-1 (SEQ ID NO: 149)
CCCGCCTCGCCACTGGGAGAGGGGCGGTGCCTGATGCCAACCATCGCCAAGGGCAACCTCGCGGGGCAGAAGTTC
CGGCGAGTAACGTCATGCCGCAAAGGACGCCGCTATTTTACGTCACTTCCTCTGCTCCCATTAG
>camelus_dromedarius/1-191_Gar-1 (SEQ ID NO: 150)
CCCGCCGGGCTGCTGGGAGAGAGGCGGTCCCTGACGCCAGCCATCTCCAAGGGCAACCCCGCGGCGGCACTTCCT
GCAGCGCCCTAAGGTAAAAGACGCCGCTATTGTACGTCACTTCCTTTGCTCGCGGTAG
>equus_caballus/1-191_Gar-1 (SEQ ID NO: 151)
AACCCGGGCGCCGGGAGAGGGCGGACCCCTGACGCCGCCGTCACCAGGGCAACCCTGCGGGCACTTCCTGCAACG
TCGCGGCAAAGGACGCCGCTATTACACGTCACTTCCTCTGCTCGTCGGTAG
>canis_lupus_dingo/1191_Gar1 (SEQ ID NO: 152)
CCGCCAGGTCCCCGGGAGAGGGGGGCGGAACTCTCACGCCAACCATCTCCCGGGGCAACAGCGCGGCCGCACTTC
CGGGAACTTCTCGACTCAACGGACGCCACTATTATACGTCATTTCCTCCGCTCCTCGTAG
>canis_lupus_familiaris/1-191_Gar-1 (SEQ ID NO: 153)
CCGCCAGGTCCCCGGGAGAGGGGGGCGGAACTCTCACGCCAACCATCTCCCGGGGCAACAGCGCGGCCGCACTTC
CGGCAACTTCTCGAGTCAACGGACGCCACTATTATACGTCATTTCCTCCGCTCCTCGTAG
>rn6 Gar-1 (SEQ ID NO: 154)
AGGCCTGACGATAGAGCCGAAGAACCCAAACCACCCCTGTCTCCAAGGGCAACGCGGCACCACACTTCCGGAAGC
GTCGAGTACGGAAGGCGCTGCTATTTTGCATCATTTCCGCCACCCCTAG
>hetGla2 Gar-1 (SEQ ID NO: 155)
CACGCCCCACTCCGGGAGAGGAGCCGGGTCTCAGACGCCTGCGGTCTCCAGGGGCAACACCGCACAACGCTTCCG
TAAACGTCATGTGCAAGGGACGTCGTTACGTCACTTCAGCGCGCCTTCCTGG
>cavPor3 Gar-1 (SEQ ID NO: 156)
CATGCGGCGTTTCGGAAGAGGAGCCCGCTTCCGGACGCCCGCCGTCTCCAGGGGCAACACTTCCGTGAACGTCAT
GTGTAAGGGACGGGTTACGTCACTTCCTGTGCTCCTTGG
>chiLan1 Gar-1 (SEQ ID NO: 157)
CATGCCCAATTCTGGAAGAGGAATCGCGTCCCTGACGCCTGTTATCTCCAGGGGCAACACTACGGCAATACTTCC
GTAAACGTCATATGTAAGGGACGCTAAACGTCACTTCCTGTACTCCTTGG
>octDeg1 Gar-1 (SEQ ID NO: 158)
CGTGCCTAACTCCGGAATTGGACCCGCGTTCCGGACACCGCTGTTTCCTGGGGCAACACTTCCGTAAACGTCATA
AGCAAGGGACGGCGACGTCACTTCCTGTGTTCCGCGG
>ochPri3 Gar-1 (SEQ ID NO: 159)
AAGGGCGAGCCCCGGGCTGACGGGCGGATCCCCAATGCCCTCCATCTCCCGGAGCAACTCGGCACTTCCGCAAAG
TTCCGCGGCCAAGGACGCCGCTTTTGTGCGTCACTTCCGCCGCTGGACGCGGG
>susScr3 Gar-1 (SEQ ID NO: 160)
CCCGCCTCGCCACTGGGAGAGGGGCGGTGCCTGATGCCAACCATCGCCAAGGGCAACCTCGCGGGGCAGAAGTTC
CGGCGAGTAACGGCATGCCGCAAAGGACGCCGCTATTTTACGTCACTTCCTCTGCTCCCATTAG
>vicPac2 Gar-1 (SEQ ID NO: 161)
CCCGCCGGGCTGCTGGGAGAGAGGCGGTCCCTGACGCCAGCCATCTCCAACGGCAACCCCGCGGCGGTACTTCCT
GCAGCGCCCTAAGGTAAAGGACGCCGCTGTTGTACGTCACTTCCTCTGCTCGCGGTAG
>camFer1 Gar-1 (SEQ ID NO: 162)
CCCGCCGGGCTGCTGGGAGAGAGGCGGTCCCTGACGCCAGCCATCTCCAAGGGCAACCCCGCGGCGGCACTTCCT
GCAGCGCCCTAAGGTAAAGGACGCCGCTATTGTACGTCACTTCCTCTACTCGCGGTAG
>turTru2 Gar-1 (SEQ ID NO: 163)
CAAGCCGATCCGCTGGGAGAGGGGCGGTCCCTGACGCCAGCCATTGCCAAGGGCAACGCCGCGGGGCGGCACTTC
CTGCAACGTCACGCTGCAAAGGACGCCGCTATTGTACGTCACTTCCTCCGCTCGCCGTAG
>orcOrcl Gar-1 (SEQ ID NO: 164)
CAAGCCGATCCGCTGGGAGAGGGGCGGTCCCTGACGCCAGCCATCGCCAAGGGCAACGCCGCGGGGCGGCACTTC
CTGCAACGTCACGCTGCAAAGGACGCCGCTATTGTACGTCACTTCCTCCGCTCGCCGTAG
>panHodl Gar-1 (SEQ ID NO: 165)
CTTGCCGGGCCGCGGGGAGAGGGCGGGCCCTGACGCTAGTTATCTCCAAGGGCAACGCCGCAGAGCGGAACTTCC
TGCAACGTCATGCTTCAAAGGACGCTGATATTGTACGTCACTTCCTCTGCTCGCAGTAG
>dasNov3 Gar-1 (SEQ ID NO: 166)
GCCGCCAGGGACTGGGAGGAACAGCCTAATTCCCAACACCTCCCGTTTCCTAGGGCAACAAAGCGGCGTCACTTC
CTGTAACGCCCTGACGCAAAGGACGTTGCCATCCTACGCCACTTCCGCTACTCTCCGGTAG
>jacJac1 Gar-1 (SEQ ID NO: 167)
CAGGGGGGAAGGGAACCCCGGCGCCAGCATCTCCCAGGGCAACGCGGCAAGCACTTCCGGGGGGAGTCTGGAGAC
GGAGACGCCGTTATTTTACGTCACTTCCGCTGTCGCTCT
>eleEdw1 Gar-1 (SEQ ID NO: 168)
TTTAGAAAAAAAATTGGACCACTAACGCCAGGCATCTCCAAGGGCAACAAAGCCGTCCCACTTCCTAACGTCATC
AGGAAAGGCACGCTGTGCTTACGTCATTTCCTTTGCTTGACGGCAG
>tupChil Gar-1 (SEQ ID NO: 169)
GGGAGGGGCGGCGCCCGGGGCCAGCTGTCTCCCGGGGCAACCTCGCGGGGCGCTTCCGGCGACGCCATGCAGCCA
CGGACGCCGTGACGTCACTTCCGCCACGCAGCGCCGG
>ancestral_sequences4/1-143 Gar-1 (SEQ ID NO: 170)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTTAG
>ancestral_sequences7/1-143 Gar-1 (SEQ ID NO: 171)
CCTACCCCGCCTCTGGAAGAGAAGGCGGATCCCTAACGCCAGCTATCTCCAAGAGCAACATTGCCGCAGCACTTC
CGGGACGTCGTGCTGGGACGCAGCTATTATACGTCACTTCCACGGCGCCGCGTTAG
>ursus_thibetanus_thibetanus/1-191 Gar-1 (SEQ ID NO: 172)
CCGCCAGGTCCCCAGGAGGGGAGGAGGGGGTGTTCACTAACGCCAGCCATCTCCCAGGGCAACACCGCGGCGGCA
CTTCCTGCAACTTCTTGATTGAAAGGACGCCACCATTATACGTCATTTCCTACGGAGGCGTAG
>zalophus_californianus/1-191 Gar-1 (SEQ ID NO: 173)
CCGCCAGGCCTCCGGGAAAGGGGGCGGATCACTAATGCCAGCCATCTCCCAGGGCAACACCGCGGGGGCACTTCC
TGCAACTTCTTGATTCAAAGGACGCCACTATTATACGTCATTTCCTATGGAGGACTAG
>mandrillus_leucophaeus/1-143 Gar-1 (SEQ ID NO: 174)
CCCACCCCGCCTCTGGAAGAGAAGGCGGATCCCTAACGCCAGCTATCTCCAAGAGCAACATTGCCGCCGCACTTC
CGGGACGTCGTGCTGCGGAGGACGCAGCTATTATGCGTCACTTCCACGGCGCGGCGTTAG
>dipodomys_ordii/1-143 Gar-1 (SEQ ID NO: 175)
CCCGCTCCGCCTCCGGCAACAGCCATCTCCACCGGCGCCAACGCCGCGGCACTTCCGGGACGCCTCGGCGCGAAG
GACGCGGACCTTTGACGTCACTTCCGCCGCCCTCAGGAG
>chinchilla_lanigera/1-143 Gar-1 (SEQ ID NO: 176)
CATGCCCAATTCTTGGAAGAGGAATCGCGTCCCTGACGCCTGTTATCTCCAGGGGCAACACTACGGCAATACTTC
CGAAACGTCATATGTAAGGGACGCTAAACGTCACTTCCACTCCTTGGCG
>octodon_degus/1-143 Gar-1 (SEQ ID NO: 177)
CGTGCCTAACTCCGGGAATTGGACCCGCGTTCCGGACACCGCTGTTTCCTGGGGCAACACTTCCGTAAACGTCAT
AAGCAAGGGACGGCGACGTCACTTCCTGTGTTCCGCGGCG
>fukomys_damarensis/1-143 Gar-1 (SEQ ID NO: 178)
NNNNNNNNNNNCCCGGGAGAGGAGCCGGGTCCCAGACCTCTGCGGTCTCCAGGGGCAACGCCACGCAACACTTCC
GAAACGTCATGTGCGAGGGACGCTGTGCTCACTTCCGGTGGGCCACTG
>heterocephalus_glaber_female/1-143 Gar-1 (SEQ ID NO: 179)
CACGCCCCACTCCAGGGAGAGGAGCCGGGTCTCAGACGCCTGCGGTCTCCAGGGGCAACACCGCACAACGCTTCC
GAAACGTCATGTGCAAGGGACGTCGTTACGTCACTTCCGCGCCTTCCTG
>ictidomys_tridecemlineatus/1143 Gar1 (SEQ ID NO: 180)
CACGCCCGACTTCTGGGAGAGGAGGCGGGTCCCTAACTCCGCTATCTCCTAGGGCAACTCGACGGCAATACTTCC
GGAACGTCCTGACGTAATGGATGCCGTTTCGCTTTACTTCCGCTTTCTCTTGCTAA
>spermophilus_dauricus/1-143 Gar-1 (SEQ ID NO: 181)
GCCCGACTTCTGGGAGAGGAGGCGGGTCCCTAACTCCGCTATCTCCTAGGGCAACACGTCGGCAATACTTCCGGA
ACGTCCTGACGTAATGGATGCCGTTTCGCTTTACTTCCGCTTTCTCTGGCTAA
>urocitellus_parryii/1-143 Gar-1 (SEQ ID NO: 182)
GCCCGACTTCTGGGAGAGGAGGCGGGTCGCTAACTCCGCTATCTCCTAGGGCAACACGACGGCAATACTTCCGGA
ACGTCCTGACGTAATGGACGCCGTTTCGCTTTACTTCCGCTTTCTCTTGCTAA
>jaculus_jaculus/1-143 Gar-1 (SEQ ID NO: 183)
NNNNNNNNNNCCCAGCGGGGGAAGGGAACCCCGGCGCCAGCATCTCCCAGGGCAACGCGGCAAGCACTTCCGGGG
GGAGTCTGGAGAAGACGCCGTTATTTTACGTCACTTCCGCTGTCGCTCTAG
>myotis_lucifugus/1-143 Gar-1 (SEQ ID NO: 184)
GAGAGAGCCGGTCTCCACCTCCGGGGATATCCCGGGGCAAAGCCGCGGTGACACTTCCGGAACGTCAGGATGCCA
CGGACGCGGCTGTTTTACGCCACTTCCTTGGCTTGTCGGAAG
>pteropus_vampyrus/1-143 Gar-1 (SEQ ID NO: 185)
GGAGAAGGGTGGGGCCTCACCCCAGACGTTTCCTAGGGCAACACCACGGCGGCACTTCCGGAACGTTGAGATGCA
ACGGACGCCGCTATTATACGTCACTTCCTCGGCTCGTCGATAG
>choloepus_hoffmanni/1-143 Gar-1 (SEQ ID NO: 186)
ACCGCTCGGGGCCTAAGAAAGATTCTTAACGCCAGTCACCTCCAAGAGAAACAGAGCAGTTGCTCTTCCTGAACG
CCACGACGCAAAGGGCGTTGCCATTGTACGTCACTTCCTCAACTCTCTGGCAG
>dasypus_novemcinctus/1-143 Gar-1 (SEQ ID NO: 187)
GCCGCCAGGGAGCTGGGAGGAAAGCCTAATTCCCAACACCTCCCGTTTCCTAGGGCAACAAAGCGGCGTCACTTC
CTGAACGCCCTGACGCAAAGGACGTTGCCATCCTACGCCACTTCCGCTACTCTCCGGTAG
>procavia_capensis/1-143 Gar-1 (SEQ ID NO: 188)
TTCTCCAGGCTCCTGGATGAAGGGGCGGATCCTTAACGCCAACCATCTCCAACGGCAACAACGCAGGGGCACTTC
CTTTACGACAGGACGCAACGGAAGCTCTTGGCGTACGTCACTTCTGCTTGTCAG
>equCab2 Gar-1 (SEQ ID NO: 189)
CCCGGGCGCCGGAGAGGGCGGGACCCCTGACGCCGCCGTCACCAAGGGCAACCCTGCGGGCACTTCCTGCAAACG
TCGCGCCAAAGGACGCCGCTATTACACGTCACTTCCTCTGCTCGTCGGTAG
>cerSim1 Gar-1 (SEQ ID NO: 190)
CCCCCGGGCCGCCGGGAGGGGGTAGACCCCCGACGCCGGCCGTCACCAGGGCAACAGCGCGCGGCACTTCCTGCA
ACGCCGCGAGGCAGAGGACGCCGCCATTATACGTCACTTCCTCTGTTCGTCGGGAG
>felCat8 Gar-1 (SEQ ID NO: 191)
CCGCCGGACCCCCGGGAGAGGGAGCGGATCACCAACGCCAACCGTCTCCCAGGGCAACACCGAGGCGGCACTTCC
GGCAAGGTCTGGATTCAAAGGACGCCACCATTATACGTCATTTCCTCTGCTCCTCAGTAG
>musFur1 Gar-1 (SEQ ID NO: 192)
CCCGCAGGCTCCCGGGAGAGGGGGCGGATCACTAACGCCAGCCATCTCCCAGGGCAACAGCCTGATGGCACTTCC
TGCAGCTTCTTTGCAGTCAAAGGACGCCACTATTAAACGTCACTTCCTACGTAGGTGAAG
>ailMel1 Gar-1 (SEQ ID NO: 193)
CCGCCAGGTCCCCAGGAGGGGAGGAGGGGGAGTTCACTAACGCCAGCCATCTCCCAGGGCAACACTGCGGCGGCA
CTTCCTGCAACTTCTTGATTGAAAGGACGCCACCATTATACGTCATTTCCTACGGAGGCGTAG
>odoRosDiv1 Gar-1 (SEQ ID NO: 194)
CCGCCAGGCTTCCGGGAAAGGGGGCGGATCACTAACGCCAGCCATCTCCCAGGGCAACACCGCGGGGGCACTTCC
GGCAACTTCTTGATTCAAAGGACGCCACTATTATACGTCATTTCCTATGGAGGACTAG
>lepWed1 Gar-1 (SEQ ID NO: 195)
CCGCCAGGCCTCCGGGAAAGGGGGCGGATCACTAACGCCAGCCATCTCCCAGGGCAACACCGCGGCGGCACTTCC
TGCAACTTCTTAGATTCAAAGGACGCCACTATTATACGTCATTTCCTACGGAGGACTAG
>pteAle1 Gar-1 (SEQ ID NO: 196)
CCTGCAGGGCTGCTAGGAGAAGGGCGGGGCCTCACCCCAGACGTTTCCTAGGGCAACACCACGGCGGCACTTCCG
GCAACGTTGAGATGCAACGGACGCCGCTATTATACGTCACTTCCTCGGCTCGTCGATAG
>pteVam1 Gar-1 (SEQ ID NO: 197)
CCTGAAGGTCTGCTAGGAGAAGGGTGGGGCCTCACCCCAGACGTTTCCTAGGGCAACACCACGGCGGCACTTCCG
GCAACGTTGAGATGCAACGGACGCCGCTATTATACGTCACTTCCTCGGCTCGTCGATAG
>eptFus1 Gar-1 (SEQ ID NO: 198)
CCCACGAGCGGCTGGAAGAGGGCCGGTCTCCACCTCCTCCCTCCCGGGACATCCCGGGGCAACACCGCGGTGACA
CTTCCTGGAACGTCAGGATGCCACGGACGCGACTATTTGACGCCACTTCCTTGGCTTGTCGGAAG
>myoLuc2 Gar-1 (SEQ ID NO: 199)
CCGACCGGCGGCCAGGAGAGAGCCGGTCTCCACCTCCGGGGATATCCCGGGGCAAAGCCGCGGTGACACTTCCTG
GAACGTCAGGATGCCACGGACGCGGCTGTTTTACGCCACTTCCTTGGCTTGTCGGAAG
>loxAfr3 Gar-1 (SEQ ID NO: 200)
CCCTCCTGGCTCCCGGGAGAGGTGGCAGAGCCCTAACGCCATCCATCTCCAAGGGCAACAGCGCAGCGGCACTTC
CTTTAACGTCATGATGCAAAGGACGCTACCTACGTCACTTCCTCTGCCCGTCGTCAG
>triMan1 Gar-1 (SEQ ID NO: 201)
TCCTCCTGGCTCCTAGAAGAGGGGGCGGATCCCTAACGCCAGCCATCTCCAAGGGCAACAACGCGCCGGCACTTC
CTGTAATGATGCAAAGGACGCTGCTGCCGTACGTCACTTCCTTGACTCGTCGGTAG
>chrAsi1 Gar-1 (SEQ ID NO: 202)
ACCTCCGGGCCTCTGGGAGAGGGGAGGATTCCTAACGCAGGTCGTTTCCAAGGGTAACAACGCAGCGGCACTTCC
TTCAACGTGTGGACGCAACGGACGCTGCACGTCACTTCCGCTGCCTGTCCGTTG
>oryAfe1 Gar-1 (SEQ ID NO: 203)
TCCTTCAGGCTGTTGGGCGTGGGGGCGGATCCCTAACGCCAGCCATCTCCAAGGGTAACAACGTGTGGGCACTTC
CACACGTCATGATGCAAAGGCCATTACTATTGTACGTCACTTCCTCTGCTTGTCGGTAA
>mouse 7sk-1 (SEQ ID NO: 204)
GAGAGTAAGCAGGCTCTTGGTAGGTATATAAGGCCATAGAATTTTGTAACTTTACACATGTGGTGACCTTATGTA
GCCGACTGTACTTGATATTATAACAAATCCTGAATCCGTTTTAGGGTTAAATAATCCTTTTTATACTCGCTTCGT
TCTAAGTTTAAATTAAAATACTTAAATTTAGGATGTTTTTACTGTTAACCAAAATGCTTTGGGGCTATGCAAAAT
ACAACAGTTTGGATTGGTTAAACCTTCCGAAGCCCCGCCCCCGACGGCCATGTCT
>CD2AP_Bidirectional_Promoter (SEQ ID NO: 205)
AGCGAGCCCAAGCTCCTCTGCACCGCTTCCTCATCCGCTCGCTGCACCTGGACGCGGTCGGCGCGCGACCCCCGG
CCGTGACGTCACCGCACCTGGCAGCAGCCGTGGGGACCGGGAGAGAGCCCGAACGCGACGGGGCGGGGTGGGGCG
GGGAGAACGAGGGCGTTCTCGCGAGATTTGCCTCCTCCCGGTCCCAGCTCCCCGCACCTTCTCGGCCTCTGTCTG
GGTCCCCACCTTAGTCTACGGTGTCGCCTTTTCTAACTGCGAGTGCTAAGGAAGAGGCGAGGGGCGGGCTCCGAG
GCTAGGCGGGCGCTCGGGGTTGGAGCCGAGGGTCTGGGCAAACCGGTGGGTCCCTCCCCACTGCGGGAGCGGCCA
GGGTGGGAAAACCGCGGTCGGGCGGGCGGGGTAGGGCCCTCCCGCCGCCGTGGCTCCTGGGGAGGCCAGGGGTGA
GGAGCTGTCGCCGCCTTTGCCTCTGCCTCGAGGGCCGCGCTGAAGAGACTGGTAGGAGAGCGCCGCGGGCGGATG
GAGGCGACTCTTCGCCCCGCCTGAGCTCAGGAGGGGCTAGCGCGGAGCGCGGGTCCCGCCTCCAGCCGCGGGAGC
GGCCGCGCGAGCCACCACTGGAGGAGGAGGAGGAGGAGCGGACGTCGGCTTCTCCCCGCGGGAGCCCCCAGC
>DCTN6_Bidirectional_Promoter (SEQ ID NO: 206)
ACGCGACGCAAACAAGAGTCGCAAGCTTCCGGGTCCCCGCCCCACCCCGGCTCCGCCCCTCCCCCAACCCTGCCA
GGCTCTCCAATCGCATGTGGAATTATCGCTCTACCCAGGCGGTGGTGTCGATCTACGTTCCAATTGGGGCCGTAC
C
>EMBP1_Bidirectional_Promoter (SEQ ID NO: 207)
AAAACCTTACACCTGCGCAAAAATAAGCCTCCCTCATAAGAAAGCCCAAAGATGTCCGGGGTCGGGGAGGAGGAA
AGTGTCTCTCATCTGTCCCATCAACGAAAATTAGTGAAATCTGCCTCAGATGAAGTGCAAAGGCCAGTCTGCAGG
GATAGTTTCAACCTCTCCCCACGCGATGGGCTACACATCACCTGCCCAAGCTCTCTCCCGACCTGCTAGAGCCTA
GAGGGCGGAGGCCGGAGAGGCTGCAGCCGGGAGTAGCACCGCACATCCGGGAACGCC
>EP400NL_Bidirectional_Promoter (SEQ ID NO: 208)
ACCCGTCTACAGTGGACACGACGAAACCAGGGACATGTCCCACCATTTCAGTGGTCACAGGCAAGAGTCTTGTGG
ATCTTCGGATCCCACGTAACATCTCATCTCCCTAGGCACCCCGACTCCCCTGCCCAATTTAAAACAGACCTCAGC
CTGCCCCATCCCGGCTGCTTTGCCTGGTGCTCTTCTAACTGCATGTTTATCTATCCTCCCCGCCTAGACTGTAGG
GCCCGCGAGGGGAGCCGCTAGCTGTGCTTGTCAGTGTGACCAGCGCTCAGCAGGTGTCCGGCGGGAGGGCGGGCA
AATACAACTCAGTGCCCACGTGCGAATGAATGAACAAACTAGTTCCGGGCGGAGCCAGAGGCGCGCGCCGGCGCG
GACCGAGGCCCGGCCCTATCCGCCCCGCCCCCTCCGCCCCGCCCCCTCCGCCACGTCCCTCCGGGTCCGCTGGGC
GCTGATTGGTCCGAGCCTCGCCTGCGCAGTGCCGGGCCGGCTCCCGCGCTTGC
>FCHO21_Bidirectional_Promoter (SEQ ID NO: 209)
CCGACTCCACTGCCGCTGGCTGGCCCTTCTCTTCCCTCTGTCCCTGGGCCAGTGCCCGTCGCACCACAAACAGTG
CGAGCAGTCTCCCCGGTGACTCCTCAAGGACCCAGTTCTCCACCATTCCTAAGAGAACACTCAACCCAGCCGCGC
CCGGGATGCAGAGAGATCTACCAACACCCGAGAATGGGGACAGGGCGCATGCGCACACCGTGGCCGTGGCGTCTA
AGTGCTCGCCCAGCTGCGGCAGCCGCTAGGTGGCGCATGCGCCCTGGAAGGTGCGGGCCGGTCTCTGGGAAGAAG
GCGGCGGCGGCGAAAGGCGGGGGTGCTGTGGGGGCCGGGCCGTGTTT
>FCHO22_Bidirectional_Promoter (SEQ ID NO: 210)
CCGACTCCACTGCCGCTGGCTGGCCCTTCTCTTCCCTCTGTCCCTGGGCCAGTGCCCGTCGCACCACAAACAGTG
CGAGCAGTCTCCCCGGTGACTCCTCAAGGACCCAGTTCTCCACCATTCCTAAGAGAACACTCAACCCAGCCGCGC
CCGGGATGCAGAGAGATCTACCAACACCCGAGAATGGGGACAGGGCGCATGCGCACACCGTGGCCGTGGCGTCTA
AGTGCTCGCCCAGCTGCGGCAGCCGCTAGGTGGCGCATGCGCCCTGGAAGGTGCGGGCCGGTCTCTGGGAAGAAG
GCGGCGGCGGCGAAAGGCGGGGGTGCTGTGGGGGCCGGGCCGTGTTTACACAGCGGCGGGCGGGCGCGGACGCGG
AACCCGGCGCGGCGGCGGCACG
>KMT5C1_Bidirectional_Promoter (SEQ ID NO: 211)
CGCGGGGCGGGAGGGGAGAGGGATGGCGGTGCGCGCGCATTCACCGCCTCCCTCCCGCCGGGTCTGGCTTTCTCC
CTCCTGTGGCCGAAGCTTTCCTCGGAGAAATAGAAGAGGGAGGCCGCGACTCTATGGTGATGGACGGAGGCCTTA
CCCAATGGAAAGAGGAGCTGTCCCAAGGCCAGGCAATCATATACGACTACTGGAGCTGGCAGAGCCCGCCCTCTT
TCCACTTGGACCTGAATAACCCGACCCAAACCGAGTTTCGCCCGGAGAGACTGCGCTTTCGGCCAATGAGTGCGT
CGATTTCGAGCCCCAGTGTGAGCGAAGGCGGGACAAGTCTCCATGGCAGCGACTAAAGGACAGCGATGTGAACCA
CTGACAACAGTTCGCGGCGTTTGACGGCGGCGGGGGCGTGGCGGGGTTTTATCTGTGTATTGACGAGAGCCGGGC
GCGGAGGGAAAGAGTGGGGCTTGGCCAATGGGAGCGCCGTGAGCTTCGTAGCAACGGAGGAGTGGCGGTGGCTGT
GGCCAATAGAAAGCCTCAGTGGCCTTGGCGGGGCTGGCCCGGAG
>KMT5C2_Bidirectional_Promoter (SEQ ID NO: 212)
CGCGGGGCGGGAGGGGAGAGGGATGGCGGTGCGCGCGCATTCACCGCCTCCCTCCCGCCGGGTCTGGCTTTCTCC
CTCCTGTGGCCGAAGCTTTCCTCGGAGAAATAGAAGAGGGAGGCCGCGACTCTATGGTGATGGACGGAGGCCTTA
CCCAATGGAAAGAGGAGCTGTCCCAAGGCCAGGCAATCATATACGACTACTGGAGCTGGCAGAGCCCGCCCTCTT
TCCACTTGGACCTGAATAACCCGACCCAAACCGAGTTTCGCCCGGAGAGACTGCGCTTTCGGCCAATGAGTGCGT
CGATTTCGAGCCCCAGTGTGAGCGAAGGCGGGACAAGTCTCCATGGCAGCGACTAAAGGACAGCGATGTGAACCA
CTGACAACAGTTCGCGGCGTTTGACGGCGGCGGGGGCGTGGCGGGGTTTTATCTGTGTATTGACGAGAGCCGGGC
GCGGAGGGAAAGAGTGGGGCTTGGCCAATGGGAGCGCCGTGAGCTTCGTAGCAACGGAGGAGTGGCGGTGGCTGT
GGCCAATAGAAAGCCTCAGTGGCCTTGGCGGGGCTGGCCCGGAGAGCAGATGGGAGGTGCGGCGACAGTGTTTGA
CGAGAGCCGAAGGAGGCTGTGGGAGGTGTTGGCGGCGGCGGCGCGGGCGCCTGAGGAGGAGGAGGAGAAGCGGGT
GAGGGGCGGCGCGGGGCCCGATCTCTGAGCCCCTTCACGGCCCCAGCCCCGCGCCGCCTTGGCTCCCCAGTCGCC
CCCTGCCCCGACTGCCCCCCACCCCGCCCGGCCCCTCCTCGTGTCCAGGCGCCCAC
>LZTR11_Bidirectional_Promoter (SEQ ID NO: 213)
TGAAGGAGCTGAGGCCCTGCTAAGTAGGAATGAGAATCCAGAGGCTCCTCGCCGGGCTGCCTCTCAGTCAGTAAG
AAAGCCAAGGGGAGAGGGGAGTTGCTGGGGGTCAGGGCTGAGGGCGCTAGCAGGAAAGGGAGCGTTGAGCCGCCT
GCAGAGGCCGCTGCGAGCCCGGAACCCTCCATGGGGGATCCCGGCAGCGGCAGACGATCCAGGCCGGAGCCACGC
GCAGACCCAGGGCATGCCGGGAACTGCGAGCCGGCCGCGGGTCTTCGGGCTGCGTGGGCCTGGGAGGCGCCGGGA
AGAGCAGTCGCGACGGGGCTAGGGACGACACACTGCATTCACTGGAAGGGACAACGCAGCGCCAGTACATAGCCT
GAAACGCTCCCCAGAAGGTCCCACGCTCGCCGCGCGGTCGACAACCGCATCCTGCGCTCGCCCGCGGTGTCTCGG
CAAGCGGTAGGCTTGTCGGGAAGAGCTGGAGGGCGCAAGTGCGGCGCTGGCCGGACGTGCCGC
>LZTR12_Bidirectional_Promoter (SEQ ID NO: 214)
TGAAGGAGCTGAGGCCCTGCTAAGTAGGAATGAGAATCCAGAGGCTCCTCGCCGGGCTGCCTCTCAGTCAGTAAG
AAAGCCAAGGGGAGAGGGGAGTTGCTGGGGGTCAGGGCTGAGGGCGCTAGCAGGAAAGGGAGCGTTGAGCCGCCT
GCAGAGGCCGCTGCGAGCCCGGAACCCTCCATGGGGGATCCCGGCAGCGGCAGACGATCCAGGCCGGAGCCACGC
GCAGACCCAGGGCATGCCGGGAACTGCGAGCCGGCCGCGGGTCTTCGGGCTGCGTGGGCCTGGGAGGCGCCGGGA
AGAGCAGTCGCGACGGGGCTAGGGACGACACACTGCATTCACTGGAAGGGACAACGCAGCGCCAGTACATAGCCT
GAAACGCTCCCCAGAAGGTCCCACGCTCGCCGCGCGGTCGACAACCGCATCCTGCGCTCGCCCGCGGTGTCTCGG
CAAGCGGTAGGCTTGTCGGGAAGAGCTGGAGGGCGCAAGTGCGGCGCTGGCCGGACGTGCCGCACCGTCAGCGCA
GGGCTCGCCGGGAAATGTGGTTTCTCCAGCCGGCCCGGGGCGGTGGCCGCAAGTTGGGCTTACAGCGCGGCCGAT
CCGGCGTGGACCCGGG
>PATJ1_Bidirectional_Promoter (SEQ ID NO: 215)
GAGTCGGGGCGAGGGGAGGGCCTGCCAGGTGAGGCGCGGTC
>PATJ2_Bidirectional_Promoter (SEQ ID NO: 216)
GAGTCGGGGCGAGGGGAGGGCCTGCCAGGTGAGGCGCGGTCACCCTGGGCCTCTCACTTCCGCCCAGGTGAGGCA
GGGCCGACACCGAGCCCGCCCGACCCGGGCTCCCACCTGCTCCTCCAGCGCACCAG
>PCNX11_Bidirectional_Promoter (SEQ ID NO: 217)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGCGGAGGGAGCGGA
>PCNX12_Bidirectional_Promoter (SEQ ID NO: 218)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGCGGAGGGAGCGGAGAGGAGG
AGCTGGAGGGGGCGCGGCTTCCTCTCGGTCG
>PCNX13_Bidirectional_Promoter (SEQ ID NO: 219)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGCGGAGGGAGCGGAGAGGAGG
AGCTGGAGGGGGCGCGGCTTCCTCTCGGTCGCTCCCTGGCGCCGGGCCTCTTTCTCTGCCTGGCCCAGGGCTGGC
GGCCGGCGGGGGTCGCGGCGGCGGCAGTGGGGGCGCTGGCGGGCCGCGGGTGGCGGGGGCCGGGCCGCGGCTCCG
GGTGTTAGGAGACAAGATGGCGGCGGCTCTCAGAAGGCCGGTCTCCTCCTCTCCGCCGTCCTCCGCCCCGCCGCT
CGCCGCCTCCTCCTCTCGGGTCTCCTCCTCCTCGTTTGCTGCCTCCTCCTCCTCCTGCAGCAGCACCAGCGACCG
CCGAAGCGCCGGCTCGCTCACCCGGAGCTCCGGAGGTGGATAGACGGGGCAGCTGCAGGCTCCGGCGACCGAGGC
CGAGCTGGGGCCGGGGCGGGGACGGCGGCGGCGGCGGCGGCGACGGCGGCGGCGCCGGGTGGGG
>PTGERN_Bidirectional_Promoter (SEQ ID NO: 220)
AATTTTTGGCATAGGCCAAGCGGCTGGTTGGTGGGGTGTTTAGCTCAGGACGAGAGGCCGAACGAGCGGGGAGTT
GGCTGAGGATAGACTAGACACGCGTGGGTGACTCCAGCGTGATGGAACGCGGGGTGTCCCGGGATAGGGCTAAAG
CGATGGGATTTCCAGACGAGTCTTTCCCAGGCCAACTTTTAAAGGTCGGAGGAAAGTTTCTCGTGGGGTGGGGGC
CCAGAGGGGATGGCAGGGTGGGCTCCGACGCCTCCTCGCCTTTAAGCGGGTGGCCCCGGCTCTTCCTCCGTTACC
TGGAGCGGGGAGGGGCTTGGGAAAGTTTGTGTTTGTTGCTGGCAAAGCGCCGGATGGGAGGCGCGGGCGGGCGCT
GCGGTTCTTCCCTTCT
>RMRP_Bidirectional_Promoter (SEQ ID NO: 221)
ACGTCCTCAGCTTCACAGAGTAGTATTTTATAGCCCTAAAGAAATTGTGTTTTATGATTAGGGTGAGAAAGTTGG
TGGCGTGAGATTAAAAAAACCGTTTTCGGGCATAACTTTCTAAGACTATAGGCTTTCAGAGGCATTGTGGCTAGC
AGAATAGCTAATAGACACGAAATGAACAAATACAGGAAAGCTAGAATGACACTATCTTATGCAAATATGGTCTGG
CCCCGCCCTACGGGGAGTGGGCGTGGCCTCCCCGGAGCCGGCCGGCCTGCTCGCGTGCGCGTGCGCGTTGGGGCG
GCCGGCCAATGCCGGACCGCTTCGGCACCGCCCGCCCGATCCCTCCACCCGTGGGCCGGCA
>RNF1871_Bidirectional_Promoter (SEQ ID NO: 222)
CCAGGACCTTGCAGGTGGAGAGCATAGTTGCCAAAATCAAGGCGGAGGAGCGCACCGCCGCTAGGATCCAGGCGG
AGAAGCCCACCGCGGCCAGGACCTAAGGATGCAGTACACTGCTGCCAGGATCTTGTCTGTGGAGCGCAGCGCGGC
CAGGACCTCCGGCTGCAGCACACCGCTGCCAGGATCTTATCGGCAGAGCGCTCCGCGGTCCGGACCCCGCCCCGT
GCGCGTCCCCGACCCCGCCCC
>RNF1872_Bidirectional_Promoter (SEQ ID NO: 223)
CCAGGACCTTGCAGGTGGAGAGCATAGTTGCCAAAATCAAGGCGGAGGAGCGCACCGCCGCTAGGATCCAGGCGG
AGAAGCCCACCGCGGCCAGGACCTAAGGATGCAGTACACTGCTGCCAGGATCTTGTCTGTGGAGCGCAGCGCGGC
CAGGACCTCCGGCTGCAGCACACCGCTGCCAGGATCTTATCGGCAGAGCGCTCCGCGGTCCGGACCCCGCCCCGT
GCGCGTCCCCGACCCCGCCCCGTGCGCGTCCCCGGCGTTGGCGTCTTCGTCCTGTTGCTGGTCTCCGTCCGGTCG
CCGGCCGTCTAGGTCTCCGGCCCTCCCCAGCCGCTCCTGCGCCCTTGCCGGCCCCGCCGCCCGCAGC
>SAMD4B1_Bidirectional_Promoter (SEQ ID NO: 224)
CGCCCACTGAGGACAGCCTTGGGTGAGGCGGGCCACCCAAGGGGGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGC
>SAMD4B2_Bidirectional_Promoter (SEQ ID NO: 225)
CGCCCACTGAGGACAGCCTTGGGTGAGGCGGGCCACCCAAGGGGGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGCGGCGGCGGTGGTC
GGTGCGGGAGGAGGGAGGGGAGCTTGCGGGCCCGAGA
>SAMD4B3_Bidirectional_Promoter (SEQ ID NO: 226)
CGCCCACTGAGGACAGCCTTGGGTGAGGCGGGCCACCCAAGGGGCGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGCGGCGGCGGTGGTC
GGTGCGGGAGGAGGGAGGGGAGCTTGCGGGCCCGAGAGGGGGCGACGGCGGCGGCGGTGGCCTGAGGAGGCCCGA
GCGGCGGCGGTGGCGGCGAAGGCCGAGGCG
>SETD1A1_Bidirectional_Promoter (SEQ ID NO: 227)
CGGAGGCGCCCCCTAGTCCCAGGCTCTGCACGCCCTGGCCCCGCCCCTTGACTCGGCCCCGCCCACAGCGGAATC
CGCAGATTCGCCAGGTCGG
>SETD1A2_Bidirectional_Promoter (SEQ ID NO: 228)
CGGAGGCGCCCCCTAGTCCCAGGCTCTGCACGCCCTGGCCCCGCCCCTTGACTCGGCCCCGCCCACAGCGGAATC
CGCAGATTCGCCAGGTCGGATCCTCAGAATTCCTCGGGTCCCTCGATACTCGGCTGAAAATTCTCATCGGACTCT
GAGAGGAGCGCTGGGCTGGAGGCATTTTCCCCAGGGACAGAAGCGGGCTATTCTCTCACTTGGGCCAGTAAGAAA
AATCCAAAAAAAGTTGTCGACTCTGCCAGCAGGGATTGGCTAACGGGCCGTTATTTTCTTGACTCCACCAAGGCG
GATGAAGGGGAGGCTACGGCTGAGGCCGGGAACAGTGGCGAATCTGCAGCCTCTCAGAATTTGGCAGTGCAAGGA
AGGGACGGGGAAGAGAAGCAAAGCGGCGCGCATCCTGTCCAGCGATTCGCCCCGCCCGCCCGGTGAATCTGCGTC
TGCAGAACGCGCCACTGAAGGTTCCCCAGCGCTGGCTGGCCTCCTCCCCTCCGCCCCGCCCCTTTTCCTCAGGGA
CTAGTCGCAGCTTTCGTCGCCGCCGATTCGTCAAGGTCCCGGGCCGCAGCATCTAGATCGTCGTGGCGAAGCCGA
CTCTCCGGGGGATGCGGCCAATCTCCAAGCTCCCTGGGCCGCAACTTCCGAGCCTCCCAGGGCGCCGGCCGAGGC
GAAGCCGCTACCCTCGGCCCCGTGGGTCCCCCGGCAGCGCCTGTGGCGAAA
>SNORD651_Bidirectional_Promoter (SEQ ID NO: 229)
GATATCTTTTTTTTTTGAAGCGAGTTTTAACAAGATCAGCTGTTTATTCATTCCACTATGGGGTTGAAGGGATCA
TTGGCCAGCTCAAGGCTTACCTTCTCTTGGGCTGAGATGCTGCTGCCAGCTCTAAAACAGCACTCTGTTCTCAAA
ACCTGGGGGAATGGAGAAGGCGCATACACCTTAGAGACTGCAGATGCAGAGCAGGACAGGCATTTCTGATGACAG
TCAATTAATGACTTTACAAATTTAAGTCCATCCTAACAAAAGCCCCTT
>SNORD652_Bidirectional_Promoter (SEQ ID NO: 230)
GATATCTTTTTTTTTTGAAGCGAGTTTTAACAAGATCAGCTGTTTATTCATTCCACTATGGGGTTGAAGGGATCA
TTGGCCAGCTCAAGGCTTACCTTCTCTTGGGCTGAGATGCTGCTGCCAGCTCTAAAACAGCACTCTGTTCTCAAA
ACCTGGGGGAATGGAGAAGGCGCATACACCTTAGAGACTGCAGATGCAGAGCAGGACAGGCATTTCTGATGACAG
TCAATTAATGACTTTACAAATTTAAGTCCATCCTAACAAAAGCCCCTTAAGACCTAATTAGAGGTAATTTTTCTA
AGTTTTTGTAAATTATTGAGGACTACAAATCTTAATTAGCTTCTCAGTAGGTTGTAATTTTTTTTTTTTTTTTGA
GATGGAGTCTCGCTGTTGCCCAGGCTGGAGTGCAGTGGCACGATTTCGACTCACTACAACCTCCGCCTCCCGGGT
TCAAGCGATTCTCCTGGCTCAGCCCCCAAAGTAGCTGGGATTACAAGTACACGCCACCACACCCGGCTAATTTTT
GTATTTTTGGTAGAGATGGGGTTTCACCATGTCGGCCAGCCAGGCTGGTCTTGAACTCCTGACCTCAGGTGATCC
ACCCACCTTAGCCTCCCAAAGTGCTGGGATTACAGGCCACTGTGCCCAGCCTCAGGGGAGTTGTAATCTCCATTT
CAGTCATATCAATTTAAACTTCACAAAGCTAAGATTACTTTTCCTTTTCACATCTGAGGAAAACTACATCTC
>SPDYA1_Bidirectional_Promoter (SEQ ID NO: 231)
AGGGAGGGGCGGGGTTCGCCGGCGCGCACTCCCAGGCAGGCCCCGCCCCCTCGGCCGGCTGTGCGCGCTGATTGG
CCCCTGCCGGCCTCGCGCTCCCTCGCTCCGGGTTGGCGGGAGACCTTAGAGC
>SPDYA2_Bidirectional_Promoter (SEQ ID NO: 232)
AGGGAGGGGCGGGGTTCGCCGGCGCGCACTCCCAGGCAGGCCCCGCCCCCTCGGCCGGCTGTGCGCGCTGATTGG
CCCCTGCCGGCCTCGCGCTCCCTCGCTCCGGGTTGGCGGGAGACCTTAGAGCGGGTACCGCTGCTGGCTAGCGAC
CGACGAGCAACCGTCTGAGGCCAGGAGCGCTGCGACGGAGCCTTGACCGCCGTTGCCCGGCCCTCTCCCGCGCAG
CCCCGGGCTTCCGCAG
>SRP_Bidirectional_Promoter (SEQ ID NO: 233)
GGTCGGATACCGGCGCAGAATAGCACTAGAAGCTGTGGTATGGTGACGTCATCAACTGGGCCAGCCCACAACGCC
TCTAAGATTTCATTTTACTCACCCAGCGAAACAACCTGACCACACTGCGCACGCGTTTCCTTTGAGCACTGCATT
CTGGGTAAACTGTCTCAAAAATTTGAAGAGCGCATGCGTGGGCCAGCTTCTTCCTTTTACCTCGTTGCACTGCTG
AGAGCAAG
>TAF151_Bidirectional_Promoter (SEQ ID NO: 234)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGC
>TAF152_Bidirectional_Promoter (SEQ ID NO: 235)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGCAGTCCGCCTCAGCCCGCCGCGCCGCCCTCAGTACAGCTCCGGC
CGCCGCGCCGCCTGGC
>TAF153_Bidirectional_Promoter (SEQ ID NO: 236)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGCAGTCCGCCTCAGCCCGCCGCGCCGCCCTCAGTACAGCTCCGGC
CGCCGCGCCGCCTGGCTTTCGTATTCGTTGTTCTCGGCGGGCTGTGGGGCCTCCGCGCCGCGGCCGTTAGTC
>TBL31_Bidirectional_Promoter (SEQ ID NO: 237)
CGAAGCACCCTCACAGCTCACGGCCCTCCCTCCAGGCCGGAAACGTCTCCGCCCGCTTCCGCTTCCCGATGCAGC
CGCCACTGCCCGAAGCAAAGATGGCGCCAAGTGCGCGGCGCCGGCGGGGACGTCACAGTGGTCGCGCGCGGTGAC
GCCATCGCAGCGCGCC
>TBL32_Bidirectional_Promoter (SEQ ID NO: 238)
CGAAGCACCCTCACAGCTCACGGCCCTCCCTCCAGGCCGGAAACGTCTCCGCCCGCTTCCGCTTCCCGATGCAGC
CGCCACTGCCCGAAGCAAAGATGGCGCCAAGTGCGCGGCGCCGGCGGGGACGTCACAGTGGTCGCGCGCGGTGAC
GCCATCGCAGCGCGCCGGGAGTGTGGCGTTCTGTGAAGAGTTCGGTGCTAACCTCCCTCACGCGGCGGTGGCTGC
CGGGACCCTAGCAGGTTTCAGCTGGAGCGGCGGCGGCGGCAAC
>ZFY1_Bidirectional_Promoter (SEQ ID NO: 239)
TTTTTTTAAAGCCAACAAAGGAGACAGTGGGGAATGCTATATGTCTGTATCTGCTTTCCTCCTCAACCCTAGGAA
TAAAGTAAACACGTTTACTGAGGGCGGGGGTCTAAGGGCCTGCAACAATGAGATCTGTCGCCTTGGCTAGGACTG
GCGCCGAGAGGCGATAGGTCTCGGGAGAGCCTGGCGCAGGGTGTGGGAGATTAGGAATCCCAGGTCCACCGGAGA
TGGCAGGGGGTGGCCTGGCCCGGTGCGGGGCCGCTTGCCTGCACGCAACCAACTAAGGCGGTGGTGCGCAAGT
>ZFY2_Bidirectional_Promoter (SEQ ID NO: 240)
TTTTTTTAAAGCCAACAAAGGAGACAGTGGGGAATGCTATATGTCTGTATCTGCTTTCCTCCTCAACCCTAGGAA
TAAAGTAAACACGTTTACTGAGGGCGGGGGTCTAAGGGCCTGCAACAATGAGATCTGTCGCCTTGGCTAGGACTG
GCGCCGAGAGGCGATAGGTCTCGGGAGAGCCTGGCGCAGGGTGTGGGAGATTAGGAATCCCAGGTCCACCGGAGA
TGGCAGGGGGTGGCCTGGCCCGGTGCGGGGCCGCTTGCCTGCACGCAACCAACTAAGGCGGTGGTGCGCAAGTAG
TGGTGACGGCGGGCGCGCGGAGAAAAGGAACGTTGTGACGGAAACTCCAGCTGCCGGAGACCCCACCGCAGTGAG
GTCACTGGACTCCCCGGACTCGGGGCGTGACCGGCGCCGACCCGGGGCGCCGAGAGGCCCACCGGGCGGAGGGGG
CCCAACTACCATCCCGCATTTTCCTGGGTCTCTCTCCCGGGCGGTGACGTGACGTGCTGACGGCGGGCCCGTGCC
GGGGAGCTGGGCCGCTTTTTGTCAGCTCCGAACTCGGCCCCTCCTCCCTCCCTCCGCCCGCCCTACCAGCCGGAG
CCCGGCCCAGTGCTCCAGAGAAAGGCCGTCCTGCAGCACCCGCCGCTGTCGCCGACCGCCCGCACATCCGTCGGG
TGAGTCCCGCGTGCCCCCGCGGCCGCGGG
>SRP-RPS29 (SEQ ID NO: 241)
CTTGCTCTCAGCAGTGCAACGAGGTAAAAGGAAGAAGCTGGCCCACGCATGCGCTCTTCAAATTTTTGAGACAGT
TTACCCAGAATGCAGTGCTCAAAGGAAACGCGTGCGCAGTGTGGTCAGGTTGTTTCGCTGGGTGAGTAAAATGAA
ATCTTAGAGGCGTTGTGGGCTGGCCCAGTTGATGACGTCACCATACCACAGCTTCTAGTGCTATTCTGCGCCGGT
ATCCGACC
>7sk1_Bidirectional_Promoter (SEQ ID NO: 242)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACT
>7Sk2_Bidirectional_Promoter (SEQ ID NO: 243)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACTGCAGTCTCCATTGGTGAGGTCGTCCCGGAGCCTCGCCCAGCTCCCGCGCGCTAGAGCCGCC
TGCTGGTCTCACCCAGCCGGGACCGCTGACCTGGCGCTTTGTGCGGCTCCAGGCCTCCGAGTGGACTCCAGAAAG
CCTGAAAAGCTATC
>7sk3_Bidirectional_Promoter (SEQ ID NO: 244)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACTGCAGTCTCCATTGGTGAGGTCGTCCCGGAGCCTCGCCCAGCTCCCGCGCGCTAGAGCCGCC
TGCTGGTCTCACCCAGCCGGGACCGCTGACCTGGCGCTTTGTGCGGCTCCAGGCCTCCGAGTGGACTCCAG
>RMRP-CCDC107 (SEQ ID NO: 245)
TGCCGGCCCACGGGTGGAGGGATCGGGCGGGCGGTGCCGAAGCGGTCCGGCATTGGCCGGCCGCCCCAACGCGCA
CGCGCACGCGAGCAGGCCGGCCGGCTCCGGGGAGGCCACGCCCACTCCCCGTAGGGCGGGGCCAGACCATATTTG
CATAAGATAGTGTCATTCTAGCTTTCCTGTATTTGTTCATTTCGTGTCTATTAGCTATTCTGCTAGCCACAATGC
CTCTGAAAGCCTATAGTCTTAGAAAGTTATGCCCGAAAACGGTTTTTTTAATCTCACGCCACCAACTTTCTCACC
CTAATCATAAAACACAATTTCTTTAGGGCTATAAAATACTACTCTGTGAAGCTGAGGACGT
>ALOXE3_Bidirectional_Promoter (SEQ ID NO: 246)
TCTTCACGAGAGCTTTACTTTTTGCTTATAAGAGGGTTCTCTATAGGAAAAGCCAGGCTTGTAGAACCGACAGAG
GATTTTATCTGTGCAGCATAGAATATTTTGGCACAGATTTGGAAGCAGCGGGTGAAGCTCGCCTGCTGCTGATTG
AGCTTTTTCTGCCTCCCGTTCTTAGAGCCCCCGCCGAGGCTGCGACGCAGGGACTGTACCATAGTAGAGGCTGGA
ACAGTGCGGCGCCGGAACCGGCCGCGCGGGGCCGCTGCGGGCTATGGGCTTCTCTGAGAGGTTCCTCCCCAGTCC
CTAGTGGCCCAGATCCCGGACACCTGGGCTCCCGCCCAGGATCCTGCAGGCCCAGGGCGGTCCTGGAGCGGAAAG
A
>CGB1_Bidirectional_Promoter (SEQ ID NO: 247)
TTGTCGGGCCCATCCTTTCTTCCCTTTGATCTTACGCAGGGTGATGGAGCCAATCACAAGAGGCTCATCCCTGAC
GTCACCCAGTCCCCAGGGCCAGTGAGGGCCCTGCGTTCCGTGGCGCCCCCTGGAGGGAGGAAGGGGAACTGCATC
TGAGAGAGAGCAGCCAATTGGGTCCGCTGACTCTGGCCAGGTTCCCGTGCCGCGTCCAACACCCCTCACTCCCTG
TCTCACTCCCCCACGGAGACTCAATTTACTTTCCATGTCCACATTCCCAGTGCTTGCGGAAGATATCCCGCTAAG
AGAGAGAC
>CGB2_Bidirectional_Promoter (SEQ ID NO: 248)
GTGTCGGGGATCTCCTTTCTTCCTTTTGACCTTACGCAGGGTGATGGAGCCAATCAGGAGAGGCTCACCCCTGAC
GTCACCCAGTCCCCAGGGCCAGTGAGGGCCCTGCGTTCCGTGGCGCCCCCTGGAGGGAGGAAGGGGAACTGTATC
TGAGAGAGAGCAGCCAATTGGGTCCGCTGACTCCGGCCGGGTTCCCGTGCCGCGTCCAACACCCCTCACTCCCTG
TCTCACTCCCCCACGGAGACTCAATTTACTTTCCATGTCCACATCCCCAGTGCTTGCGGAAGATATCCCGCTAAG
AGAGAGAC
>Med16-1_Bidirectional_Promoter (SEQ ID NO: 249)
GAATATTGAGTTCCACCACCAGCTATTTAAAGCCCCTGGAACAAATGTCTGTACACATAGGCCGACTTCTCTTAA
ATGACCTAGAGATTTAACCTCTATTTATATTAGCCCAATGTGTAATGCAACTAACGTAGTTATTGACTGGAGTTG
AGAAAGTGCTCGTTGTTCTACCAAATATAGCTACGGTGGCTGCTGGGAATTACTGGAAATGGTCGTATGCAAATA
GCCCCGGAGGCGGGGCAGAGCCTGAGCCGCACCGCCCTCCCAGAAGTCTTTGGGAGGCGGCCCCACGCCTCAGGC
GACTGGTTGTTACCGAGGAAGATGGCGGCGCCAGACCCGAGGCGCTAGGGAAGATCGCACCGCGGACGCCCGCTG
AGCTTGGCGCACGGGCCAGGAGCTGGTGACTGCCCTC
>Med16-2_Bidirectional_Promoter (SEQ ID NO: 250)
GAATATTGAGTTCCACCACCAGCTATTTAAAGCCCCTGGAACAAATGTCTGTACACATAGGCCGACTTCTCTTAA
ATGACCTAGAGATTTAACCTCTATTTATATTAGCCCAATGTGTAATGCAACTAACGTAGTTATTGACTGGAGTTG
AGAAAGTGCTCGTTGTTCTACCAAATATAGCTACGGTGGCTGCTGGGAATTACTGGAAATGGTCGTATGCAAATA
GCCCCGGAGGCGGGGCAGAGCCTGAGCCGCACCGCCCTCCCAGAAGTCTTTGGGAGGCGGCCCCACGCCTCAGGC
GACTGGTTGTTACCGAGGAAGATGGCGGCGCCAGACCCGAGGCGCTAGGGAAGATCGCACCGCGGACGCCCGCTG
AGCTTGGCGCACGGGC
>DPP9-1_Bidirectional_Promoter (SEQ ID NO: 251)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCGGTCTGTGTCCCACGCCTGCAGCTGGAATGGAGGCTCTCTGGACCCTTTAGAAGG
CACCCCTGCCCTCCTGAGGTCAGCTGAGCGGTTA
>DPP9-2_Bidirectional_Promoter (SEQ ID NO: 252)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCGGTCTGTGTCCCACGCCTGCAGCTGGAATGGAGGCTCTCTGGACCCTTTAGAAG
>DPP9-3_Bidirectional_Promoter (SEQ ID NO: 253)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCG
>SNORD13_C8orf41 (SEQ ID NO: 254)
TCCTGACTGCAGCACCAGAAGGCTGGTCTCTCCCACAGAACGAGGATGGAGGCGGGGAGGGATCCGTTGAAGAGG
GAAGGAGCGATCACCCAAAGAGAACTAAAATCAAATAAAATAAAACAGAGAGATGTCTTGGAGGAGGGGGCGAGT
CTGACCGGGATAAGAATAAAGAGAAAGGGTGAACCCGGGAGGCGGAGTTTGCAGTGAGCCGAGATCGCGCCACTG
CACTCCAGCCTGGGCGACAGAGTGAGACTCCGTCTCAGTAAAAAAAAAAAAAAAAAAAAGAATAAAGAGGAAAGG
ACGCAAGAAAGGGAAAGGGGACTCTCAGGGAGTAAAAGAGTCTTACACTTTTAACAGTGACGTTAAAAGACTACT
GTTGCCTTTCTGAAGACTAAAAAGAAAAAAAACTTAAAAATTTAAAGAAATAAACTTCTGAGCCATGTCACCAAC
TTAACCACCCCCAGGTACCTGCAACGGCTCGCGCCCGCCGGTGTCTAACAGGATCCGGACCTAGCTCATATTGCT
GCCGCAAAACGCAAGGCTAGCTTCCGCCAGTACTGCCGCAACACCTTCTTATTTCACGACGTATGGTCGTAAAGC
AATAAAGATCCAGGCTCGGGAAAATGACGGAGAGGTGGAACTATAGAGAATAAATTTGCATATATAATAATCCGC
TCGCTAATTGTGTTTCTGTTTTCCTTTGCTAAGGTAGAAACAAAAGAATAATCACAGAATCTCAGTGGGACTTTG
AAAATATCCAGGATTTTATACGTGAAGAATGGATGTATCGCATTACGGTAGTCACCCTATGTGTAAATTAGTGGC
ACATACTTGGCACTCCTTAATGTCAACTATAAGATG
>THEM259_Bidirectional_Promoter (SEQ ID NO: 255)
GACTCAAGGGTTACTGTCACACCTATTTTAAGCCCTTCAATCAAATCATCTTTTGGTTAGGATAACTTATGGTCG
GTTTCATATTTAGCATAATTTCCTACAGTGGTATGTTGCAGAACAACTTTCGTGCTTACGCTTACTTTGATGTCT
TCGATCACGTAAAATCCCATATCTTATCGTAATTTTACCGCCTTATACTGGCCTCATAGCCGCGGTGGATTGTGG
GTGCCAATATGCAAAAGAGGTGGCCCAGATGCAGGCCCGCCCCCTGGAGCGGCCGAGGTAGGGGGTGAGGCCTCC
GCGGGCGCCGCTGGCATCCCAGCGTTCTCTGCGGGCGCAGGGGGGCCGCTCTTGCCCGGCGTGGCGACTCGCTAG
CGTCAGCAGCGCCGCAGCCGGACGAGAAAGCGGAAGATGGCGGCGGCGGCCGGGAGGCCGTGAGGAGAGCGGCGG
CTGCGAGGGCGGCCGATGGCGGCCGGGAGGCGCCCTCGGACACTTGCGGGTCGTTAGGGCGCGACGCTGGGAGGC
>CFTR (deltaR) (SEQ ID NO: 926)
MQRSPLEKASVVSKLFFSWTRPILRKGYRQRLELSDIYQIPSVDSADNLSEKLEREWDRELASKKNPKLINALRR
CFFWRFMFYGIFLYLGEVTKAVQPLLLGRIIASYDPDNKEERSIAIYLGIGLCLLFIVRTLLLHPAIFGLHHIGM
QMRIAMFSLIYKKTLKLSSRVLDKISIGQLVSLLSNNLNKFDEGLALAHFVWIAPLQVALLMGLIWELLQASAFC
GLGFLIVLALFQAGLGRMMMKYRDQRAGKISERLVITSEMIENIQSVKAYCWEEAMEKMIENLRQTELKLTRKAA
YVRYFNSSAFFFSGFFVVFLSVLPYALIKGIILRKIFTTISFCIVLRMAVTRQFPWAVQTWYDSLGAINKIQDEL
QKQEYKTLEYNLTTTEVVMENVTAFWEEGFGELFEKAKQNNNNRKTSNGDDSLFFSNFSLLGTPVLKDINFKIER
GQLLAVAGSTGAGKTSLLMVIMGELEPSEGKIKHSGRISFCSQFSWIMPGTIKENIIFGVSYDEYRYRSVIKACQ
LEEDISKFAEKDNIVLGEGGITLSGGQRARISLARAVYKDADLYLLDSPFGYLDVLTEKEIFESCVCKLMANKTR
ILVTSKMEHLKKADKILILHEGSSYFYGTFSELQNLQPDFSSKLMGCDSFDQFSAERRNSILTETLHRFSLEGDA
PVSWTETKKQSFKQTGEFGEKRKNSILNPINSITLQARRRQSVLNLMTHSVNQGQNIHRKTTASTRKVSLAPQAN
LTELDIYSRRLSQETGLEISEEINEEDLKECFFDDMESIPAVTTWNTYLRYITVHKSLIFVLIWCLVIFLAEVAA
SLVVLWLLGNTPLQDKGNSTHSRNNSYAVIITSTSSYYVFYIYVGVADTLLAMGFFRGLPLVHTLITVSKILHHK
MLHSVLQAPMSTLNTLKAGGILNRFSKDIAILDDLLPLTIFDFIQLLLIVIGAIAVVAVLQPYIFVATVPVIVAF
IMLRAYFLQTSQQLKQLESEGRSPIFTHLVTSLKGLWTLRAFGRQPYFETLFHKALNLHTANWFLYLSTLRWFQM
RIEMIFVIFFIAVTFISILTTGEGEGRVGIILTLAMNIMSTLQWAVNSSIDVDSLMRSVSRVFKFIDMPTEGKPT
KSTKPYKNGQLSKVMIIENSHVKKDDIWPSGGQMTVKDLTAKYTEGGNAILENISFSISPGQRVGLLGRTGSGKS
TLLSAFLRLLNTEGEIQIDGVSWDSITLQQWRKAFGVIPQKVFIFSGTFRKNLDPYEQWSDQEIWKVADEVGLRS
VIEQFPGKLDFVLVDGGCVLSHGHKQLMCLARSVLSKAKILLLDEPSAHLDPVTYQIIRRTLKQAFADCTVILCE
HRIEAMLECQQFLVIEENKVRQYDSIQKLLNERSLFRQAISPSDRVKLEPHRNSSKCKSKPQIAALKEETEEEVQ
DTRL
>ATP7B (SEQ ID NO: 927)
MPEQERQITAREGASRKILSKLSLPTRAWEPAMKKSFAFDNVGYEGGLDGLGPSSQVATSTVRILGMTCQSCVKS
IEDRISNLKGIISMKVSLEQGSATVKYVPSVVCLQQVCHQIGDMGFEASIAEGKAASWPSRSLPAQEAVVKLRVE
GMTCQSCVSSIEGKVRKLQGVVRVKVSLSNQEAVITYQPYLIQPEDLRDHVNDMGFEAAIKSKVAPLSLGPIDIE
RLQSTNPKRPLSSANQNENNSETLGHQGSHVVTLQLRIDGMHCKSCVLNIEENIGQLLGVQSIQVSLENKTAQVK
YDPSCTSPVALQRAIEALPPGNFKVSLPDGAEGSGTDHRSSSSHSPGSPPRNQVQGTCSTTLIAIAGMTCASCVH
SIEGMISQLEGVQQISVSLAEGTATVLYNPSVISPEELRAAIEDMGFEASVVSESCSTNPLGNHSAGNSMVQTTD
GTPTSVQEVAPHTGRLPANHAPDILAKSPQSTRAVAPQKCFLQIKGMTCASCVSNIERNLQKEAGVLSVLVALMA
GKAEIKYDPEVIQPLEIAQFIQDLGFEAAVMEDYAGSDGNIELTITGMTCASCVHNIESKLTRINGITYASVALA
TSKALVKFDPEIIGPRDIIKIIEEIGFHASLAQRNPNAHHLDHKMEIKQWKKSFLCSLVFGIPVMALMIYMLIPS
NEPHQSMVLDHNIIPGLSILNLIFFILCTFVQLLGGWYFYVQAYKSLRHRSANMDVLIVLATSIAYVYSLVILVV
AVAEKAERSPVTFFDTPPMLFVFIALGRWLEHLAKSKTSEALAKLMSLQATEATVVTLGEDNLIIREEQVPMELV
QRGDIVKVVPGGKFPVDGKVLEGNTMADESLITGEAMPVTKKPGSTVIAGSINAHGSVLIKATHVGNDTTLAQIV
KLVEEAQMSKAPIQQLADRFSGYFVPFIIIMSTLTLVVWIVIGFIDFGVVQRYFPNPNKHISQTEVIIRFAFQTS
ITVLCIACPCSLGLATPTAVMVGTGVAAQNGILIKGGKPLEMAHKIKTVMFDKTGTITHGVPRVMRVLLLGDVAT
LPLRKVLAVVGTAEASSEHPLGVAVTKYCKEELGTETLGYCTDFQAVPGCGIGCKVSNVEGILAHSERPLSAPAS
HLNEAGSLPAEKDAVPQTFSVLIGNREWLRRNGLTISSDVSDAMTDHEMKGQTAILVAIDGVLCGMIAIADAVKQ
EAALAVHTLQSMGVDVVLITGDNRKTARAIATQVGINKVFAEVLPSHKVAKVQELQNKGKKVAMVGDGVNDSPAL
AQADMGVAIGTGTDVAIEAADVVLIRNDLLDVVASIHLSKRTVRRIRINLVLALIYNLVGIPIAAGVEMPIGIVL
QPWMGSAAMAASSVSVVLSSLQLKCYKKPDLERYEAQAHGHMKPLTASQVSVHIGMDDRWRDSPRATPWDQVSYV
SQVSLSSLTSDKPSRHSAAADDDGDKWSLLLNGRDEEQYI
>ATP7A (SEQ ID NO: 928)
MDPSMGVNSVTISVEGMTCNSCVWTIEQQIGKVNGVHHIKVSLEEKNATIIYDPKLQTPKTLQEAIDDMGEDAVI
HNPDPLPVLTDTLFLTVTASLTLPWDHIQSTLLKTKGVTDIKIYPQKRTVAVTIIPSIVNANQIKELVPELSLDT
GTLEKKSGACEDHSMAQAGEVVLKMKVEGMTCHSCTSTIEGKIGKLQGVQRIKVSLDNQEATIVYQPHLISVEEM
KKQIEAMGFPAFVKKQPKYLKLGAIDVERLKNTPVKSSEGSQQRSPSYTNDSTATFIIDGMHCKSCVSNIESTLS
ALQYVSSIVVSLENRSAIVKYNASSVTPESLRKAIEAVSPGLYRVSITSEVESTSNSPSSSSLQKIPLNVVSQPL
TQETVINIDGMTCNSCVQSIEGVISKKPGVKSIRVSLANSNGTVEYDPLLTSPETLRGAIEDMGFDATLSDTNEP
LVVIAQPSSEMPLLTSTNEFYTKGMTPVQDKEEGKNSSKCYIQVTGMTCASCVANIERNLRREEGIYSILVALMA
GKAEVRYNPAVIQPPMIAEFIRELGFGATVIENADEGDGVLELVVRGMTCASCVHKIESSLTKHRGILYCSVALA
TNKAHIKYDPEIIGPRDIIHTIESLGFEASLVKKDRSASHLDHKREIRQWRRSFLVSLFFCIPVMGLMIYMMVMD
HHFATLHHNQNMSKEEMINLHSSMFLERQILPGLSVMNLLSFLLCVPVQFFGGWYFYIQAYKALKHKTANMDVLI
VLATTIAFAYSLIILLVAMYERAKVNPITFFDTPPMLFVFIALGRWLEHIAKGKTSEALAKLISLQATEATIVTL
DSDNILLSEEQVDVELVQRGDIIKVVPGGKFPVDGRVIEGHSMVDESLITGEAMPVAKKPGSTVIAGSINQNGSL
LICATHVGADTTLSQIVKLVEEAQTSKAPIQQFADKLSGYFVPFIVFVSIATLLVWIVIGFLNFEIVETYFPGYN
RSISRTETIIRFAFQASITVLCIACPCSLGLATPTAVMVGTGVGAQNGILIKGGEPLEMAHKVKVVVFDKTGTIT
HGTPVVNQVKVLTESNRISHHKILAIVGTAESNSEHPLGTAITKYCKQELDTETLGTCIDFQVVPGCGISCKVTN
IEGLLHKNNWNIEDNNIKNASLVQIDASNEQSSTSSSMIIDAQISNALNAQQYKVLIGNREWMIRNGLVINNDVN
DFMTEHERKGRTAVLVAVDDELCGLIAIADTVKPEAELAIHILKSMGLEVVLMTGDNSKTARSIASQVGITKVFA
EVLPSHKVAKVKQLQEEGKRVAMVGDGINDSPALAMANVGIAIGTGTDVAIEAADVVLIRNDLLDVVASIDLSRK
TVKRIRINFVFALIYNLVGIPIAAGVFMPIGLVLQPWMGSAAMAASSVSVVLSSLFLKLYRKPTYESYELPARSQ
IGQKSPSEISVHVGIDDTSRNSPKLGLLDRIVNYSRASINSLLSDKRSLNSVVTSEPDKHSLLVGDFREDDDTAL
>AGL (SEQ ID NO: 929)
MGHSKQIRILLLNEMEKLEKTLERLEQGYELQFRLGPTLQGKAVTVYTNYPFPGETFNREKFRSLDWENPTERED
DSDKYCKLNLQQSGSFQYYFLQGNEKSGGGYIVVDPILRVGADNHVLPLDCVTLQTFLAKCLGPFDEWESRLRVA
KESGYNMIHFTPLQTLGLSRSCYSLANQLELNPDFSRPNRKYTWNDVGQLVEKLKKEWNVICITDVVYNHTAANS
KWIQEHPECAYNLVNSPHLKPAWVLDRALWRFSCDVAEGKYKEKGIPALIENDHHMNSIRKIIWEDIFPKLKLWE
FFQVDVNKAVEQFRRLLTQENRRVTKSDPNQHLTIIQDPEYRRFGCTVDMNIALTTFIPHDKGPAAIEECCNWFH
KRMEELNSEKHRLINYHQEQAVNCLLGNVFYERLAGHGPKLGPVTRKHPLVTRYFTFPFEEIDFSMEESMIHLPN
KACFLMAHNGWVMGDDPLRNFAEPGSEVYLRRELICWGDSVKLRYGNKPEDCPYLWAHMKKYTEITATYFQGVRL
DNCHSTPLHVAEYMLDAARNLQPNLYVVAELFTGSEDLDNVFVTRLGISSLIREAMSAYNSHEEGRLVYRYGGEP
VGSFVQPCLRPLMPAIAHALFMDITHDNECPIVHRSAYDALPSTTIVSMACCASGSTRGYDELVPHQISVVSEER
FYTKWNPEALPSNTGEVNFQSGIIAARCAISKLHQELGAKGFIQVYVDQVDEDIVAVTRHSPSIHQSVVAVSRTA
FRNPKTSFYSKEVPQMCIPGKIEEVVLEARTIERNTKPYRKDENSINGTPDITVEIREHIQLNESKIVKQAGVAT
KGPNEYIQEIEFENLSPGSVIIFRVSLDPHAQVAVGILRNHLTQFSPHFKSGSLAVDNADPILKIPFASLASRLT
LAELNQILYRCESEEKEDGGGCYDIPNWSALKYAGLQGLMSVLAEIRPKNDLGHPFCNNLRSGDWMIDYVSNRLI
SRSGTIAEVGKWLQAMFFYLKQIPRYLIPCYFDAILIGAYTTLLDTAWKQMSSFVQNGSTFVKHLSLGSVQLCGV
GKFPSLPILSPALMDVPYRLNEITKEKEQCCVSLAAGLPHESSGIFRCWGRDTFIALRGILLITGRYVEARNIIL
AFAGTLRHGLIPNLLGEGIYARYNCRDAVWWWLQCIQDYCKMVPNGLDILKCPVSRMYPTDDSAPLPAGTLDQPL
FEVIQEAMQKHMQGIQFRERNAGPQIDRNMKDEGENITAGVDEETGFVYGGNRFNCGTWMDKMGESDRARNRGIP
ATPRDGSAVEIVGLSKSAVRWLLELSKKNIFPYHEVTVKRHGKAIKVSYDEWNRKIQDNFEKLFHVSEDPSDLNE
KHPNLVHKRGIYKDSYGASSPWCDYQLRPNFTIAMVVAPELFTTEKAWKALEIAEKKLLGPLGMKTLDPDDMVYC
GIYDNALDNDNYNLAKGFNYHQGPEWLWPIGYFLRAKLYFSRLMGPETTAKTIVLVKNVLSRHYVHLERSPWKGL
PELTNENAQYCPFSCETQAWSIATILETLYDL
>CPS1_1 (SEQ ID NO: 930)
MAEAHQAVAFQFTVTPDGVDFRLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAYLESVRPLLDDEEYYRMELLAKEFQDKTAPRLQKYLVLKSWWASNYVSDWWEEYIYLRGRSPLMVNSNYYVM
DLVLIKNTDVQAARLGNIIHAMIMYRRKLDREEIKPVMALGIVPMCSYQMERMENTTRIPGKDTDVLQHLSDSRH
VAVYHKGRFFKLWLYEGARLLKPQDLEMQFQRILDDPSPPQPGEEKLAALTAGGRVEWAQARQAFFSSGKNKAAL
EAIERAAFFVALDEESYSYDPEDEASLSLYGKALLHGNCYNRWFDKSFTLISFKNGQLGLNAEHAWADAPIIGHL
WEFVLGTDSFHLGYTETGHCLGKPNPALAPPTRLQWDIPKQCQAVIESSYQVAKALADDVELYCFQFLPFGKGLI
KKCRTSPDAFVQIALQLAHFRDRGKFCLTYEASMTRMFREGRTETVRSCTSESTAFVQAMMEGSHTKADLRDLFQ
KAAKKHQNMYRLAMTGAGIDRHLFCLYLVSKYLGVSSPFLAEVLSEPWRLSTSQIPQSQIRMEDPEQHPNHLGAG
GGFGPVADDGYGVSYMIAGENTIFFHISSKFSSSETNAQRFGNHIRKALLDIADLFQVPKAYS
>CPS1_2 (SEQ ID NO: 931)
MAEAHQAVAFQFTVTPDGVDFRLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAMCIRLLSSRHPMLYSFQTSLPKLPVPRVSATIQRYLESVRPLLDDEEYYRMELLAKEFQDKTAPRLQKYLVL
KSWWASNYVSDWWEEYIYLRGRSPLMVNSNYYVMDLVLIKNTDVQAARLGNIIHAMIMYRRKLDREEIKPVMALG
IVPMCSYQMERMENTTRIPGKDTDVLQHLSDSRHVAVYHKGRFFKLWLYEGARLLKPQDLEMQFQRILDDPSPPQ
PGEEKLAALTAGGRVEWAQARQAFFSSGKNKAALEAIERAAFFVALDEESYSYDPEDEASLSLYGKALLHGNCYN
RWFDKSFTLISFKNGQLGLNAEHAWADAPIIGHLWEFVLGTDSFHLGYTETGHCLGKPNPALAPPTRLQWDIPKQ
CQAVIESSYQVAKALADDVELYCFQFLPFGKGLIKKCRTSPDAFVQIALQLAHFRDRGKFCLTYEASMTRMFREG
RTETVRSCTSESTAFVQAMMEGSHTKADLRDLFQKAAKKHQNMYRLAMTGAGIDRHLFCLYLVSKYLGVSSPFLA
EVLSEPWRLSTSQIPQSQIRMFDPEQHPNHLGAGGGFGPVADDGYGVSYMIAGENTIFFHISSKFSSSETNAQRF
GNHIRKALLDIADLFQVPKAYS
>CPS1_3 (SEQ ID NO: 932)
MAEAHQAVAFQFTVTPDGVDERLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAMCIRLLSSRHPMLYSFQTSLPKLPVPRVSATIQRYLESVRPLLDDEEYYRMELLAKEFQDKTAPRLQKYLVL
KSWWASNYVSDWWEEYIYLRGRSPLMVNSNYYVMDLVLIKNTDVQAARLGNIIHAMIMYRRKLDREEIKPVMALG
IVPMCSYQMERMENTTRIPGKDTDVLQHLSDSRHVAVYHKGRFFKLWLYEGARLLKPQDLEMQFQRILDDPSPPQ
PGEEKLAALTAGGRVEWAQARQAFFSSGKNKAALEAIERAAFFVALDEESYSYDPEDEASLSLYGKALLHGNCYN
RWFDKSFTLISFKNGQLGLNAEHAWADAPIIGHLWEFVLGTDSFHLGYTETGHCLGKPNPALAPPTRLQWDIPKQ
CQAVIESSYQVAKALADDVELYCFQFLPFGKGLIKKCRTSPDAFVQIALQLAHFRDRGKFCLTYEASMTRMFREG
RTETVRSCTSESTAFVQAMMEGSHTKADLRDLFQKAAKKHQNMYRLAMTGAGIDRHLFCLYLVSKYLGVSSPFLA
EVLSEPWRLSTSQIPQSQIRMEDPEQHPNHLGAGGGFGPVADDGYGVSYMIAGENTIFFHISSKFSSSETNAQRF
GNHIRKALLDIADLFQVPKAYS
>CPS1_4 (SEQ ID NO: 933)
MAEAHQAVAFQFTVTPDGVDFRLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAMCIRLLSSRHPMLYSFQTSLPKLPVPRVSATIQRYLESVRPLLDDEEYYRMELLAKEFQDKTAPRLQKYLVL
KSWWASNYVSDWWEEYIYLRGRSPLMVNSNYYVMDLVLIKNTDVQAARLGNIIHAMIMYRRKLDREEIKPVMALG
IVPMCSYQMERMENTTRIPGKDTDVLQHLSDSRHVAVYHKGREFKLWLYEGARLLKPQDLEMQFQRILDDPSPPQ
PGEEKLAALTAGGRVEWAQARQAFFSSGKNKAALEAIERAAFFVALDEESYSYDPEDEASLSLYGKALLHGNCYN
RWFDKSFTLISFKNGQLGLNAEHAWADAPIIGHLWEFVLGTDSFHLGYTETGHCLGKPNPALAPPTRLQWDIPKQ
CQAVIESSYQVAKALADDVELYCFQFLPFGKGLIKKCRTSPDAFVQIALQLAHFRDRGKFCLTYEASMTRMFREG
RTETVRSCTSESTAFVQAMMEGSHTKADLRDLFQKAAKKHQNMYRLAMTGAGIDRHLFCLYLVSKYLGVSSPFLA
EVLSEPWRLSTSQIPQSQIRMFDPEQHPNHLGAGGGFGPVADDGYGVSYMIAGENTIFFHISSKFSSSETNAQRF
GNHIRKALLDIADLFQVPKAYS
>CPS1_5 (SEQ ID NO: 934)
MAEAHQAVAFQFTVTPDGVDFRLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAMCIRLLSSRHPMLYSFQTSLPKLPVPRVSATIQRYLESVRPLLDDEEYYRMELLAKEFQDKTAPRLQKYLVL
KSWWASNYVSDWWEEYIYLRGRSPLMVNSNYYVMDLVLIKNTDVQAARLGNIIHAMIMYRRKLDREEIKPVMALG
IVPMCSYQMERMENTTRIPGKDTDVLQHLSDSRHVAVYHKGRFFKLWLYEGARLLKPQDLEMQFQRILDDPSPPQ
PGEEKLAALTAGGRVEWAQARQAFFSSGKNKAALEAIERAAFFVALDEESYSYDPEDEASLSLYGKALLHGNCYN
RWFDKSFTLISFKNGQLGLNAEHAWADAPIIGHLWEFVLGTDSFHLGYTETGHCLGKPNPALAPPTRLQWDIPKQ
CQAVIESSYQVAKALADDVELYCFQFLPFGKGLIKKCRTSPDAFVQIALQLAHFRDRGKFCLTYEASMTRMFREG
RTETVRSCTSESTAFVQAMMEGSHTKADLRDLFQKAAKKHQNMYRLAMTGAGIDRHLFCLYLVSKYLGVSSPFLA
EVLSEPWRLSTSQIPQSQIRMEDPEQHPNHLGAGGGFGPVADDGYGVSYMIAGENTIFFHISSKFSSSETNAQRF
GNHIRKALLDIADLFQVPKAYS
>CPS1_6 (SEQ ID NO: 935)
MAEAHQAVAFQFTVTPDGVDFRLSREALKHVYLSGINSWKKRLIRIKNGILRGVYPGSPTSWLVVIMATVGSSFC
NVDISLGLVSCIQRCLPQGCGPYQTPQTRALLSMAIFSTGVWVTGIFFFRQTLKLLLCYHGWMFEMHGKTSNLTR
IWAMCIRLLSSRHPMLYSFQTSLPKLPVPRVSATIQRYLESVRPLLDDEEYYRMELLAKEFQDKTAPRLQKYLVL
KSWWASNYVSDWWEEYIYLRGRSPLMVNSNYYVMDLVLIKNTDVQAARLGNIIHAMIMYRRKLDREEIKPVMALG
IVPMCSYQMERMENTTRIPGKDTDVLQHLSDSRHVAVYHKGRFFKLWLYEGARLLKPQDLEMQFQRILDDPSPPQ
PGEEKLAALTAGGRVEWAQARQAFFSSGKNKAALEAIERAAFFVALDEESYSYDPEDEASLSLYGKALLHGNCYN
RWFDKSFTLISFKNGQLGLNAEHAWADAPIIGHLWEFVLGTDSFHLGYTETGHCLGKPNPALAPPTRLQWDIPKQ
CQAVIESSYQVAKALADDVELYCFQFLPFGKGLIKKCRTSPDAFVQIALQLAHFRDRGKFCLTYEASMTRMFREG
RTETVRSCTSESTAFVQAMMEGSHTKADLRDLFQKAAKKHQNMYRLAMTGAGIDRHLFCLYLVSKYLGVSSPFLA
EVLSEPWRLSTSQIPQSQIRMFDPEQHPNHLGAGGGFGPVADDGYGVSYMIAGENTIFFHISSKFSSSETNAQRF
GNHIRKALLDIADLFQVPKAYS
>H1_2-H1_83 (SEQ ID NO: 936)
TGGCAAACACCGCCGGCGCAAACAGCATTTATAATGCGCTCATACCTAAAGCCACTTGTCGGTTACGGTGACTTC
CCAACAAGACATTGCGACATGCAAATACTACAGTGCGTCCCGCCCCCTGGTGTAGTTCCACGCTGGGACGCACAC
GCACTACGGTTCCCGCCTTTAGACGACTGCGCTGGCGATTCCTGGGAGAGGACTGATGACGTCAGCGTTCGGGCT
CC
>H1_2-H1_90 (SEQ ID NO: 937)
TGGCAAACACTGCCGGCTCAAACAGCATTTATAATGCGCTCATACCTAAAGCCACTTGTCGGTTACGGTGACTTC
CCAACAAGACATTGCGACATGCAAATACTGCGGTGCGTCCCGCCCCCTGGTGTAGTTCCACGCTGGGACGCACAC
GCACTACGGTTCCCGCCTTTAGACGACTGCGCCGGCGATTCCTGGGAGAGGACTGATGACGTCAGCGTTCGGGCT
CC
>H1_2-H1_92 (SEQ ID NO: 938)
TGGCAAACAACGCCGGCGCAAACAGCATTTATAATGCGCTCATACCTAAAGCCACTTGTCGGTTACGGTGACTTC
CCAACAAGACATTGCGACATGCAAATATTACAGTGCGTCCCGCCCCCTGGTGTAGTTCCACGCTAGGACGCACAC
GCACTACGGTTCCCGCCTTTAGACGACTGCGCTGGCGATTCCTGGGAGCGGACTGATGACGTCAGCGTTCGGGCT
CC
>H1_2-H1_95 (SEQ ID NO: 939)
TGGCAAAAACTGACGGCTCAAGCAGCATTTATAAGGCTCCCATACCTAAAGACATTTGTCGGTTATGGTGACTTC
CCCACAAGACATTGCGACATGCAAATACTGCGGAGCGTACCCCCTCCCCCTGGCGCAACTCCTCGCTGGGACGCA
CGCGCGCTACGTGTTCCCGCCTTTAGTGACGTCTGCGCCGGCGATTCCTGGGAGAGGGTTGATGACGTCAGCGTT
CGGGCTCC
>H1_2-H1_98 (SEQ ID NO: 940)
TGGGAAAAAGTGGCGGCTCACGCAGCATTTATAAGGCTCCCATACCTAAAGACATTTCACGGTTATGGTGACTTC
CCCACAAGACATAGCGACATGCAAATATTGCGGAGCGTACGCGCCTCCCCCTGTCCTGTGCAGGCATCTTCTCAG
CCAGGACGCACGCGCGCTGCGTGTTCCCGCCCTGAGTGACTTCTGCGCCGGCGATTTCCTGGGAGGAGGGTTGAT
GACGTCAACGTTCGGGCTCC
>H1_2-H1_104 (SEQ ID NO: 941)
TGGCAAAAACTGCCGGCTCAAGCAGCATTTATAATGCGCCCATACCTAAAGCCACTTGTCGGTTACGGTGACTTC
CCAACAAGACATTGCGACATGCAAATACTGCGGTGCGTCCCTCCCCCTGGCGTAACTCCACGCTGGGACGCACGC
GCGCTACGTGTTCCCGCCTTTACTGACGTCTGCGCCGGCGATTCCTGGGAGAGGGTTGATGACGTCAGCGTTCGG
GCTCC
>H1_2-H1_113 (SEQ ID NO: 942)
TGGGAAAAAGTGGCGGCTCACGCAGCATTTATAAGGCTCCCATACCTAAAGACATTTCACGGTTATGGTGACTTC
CCCACAAGACATTGCGACATGCAAATATTGCGGAGCGTACGCCCTCCCCCTGTCCTGTGCAGGCATCTTCTCGCC
AGGACGCACGCGCGCTGCGTGTTCCCGCCTTGAGTGACTTCTGCGCCGGCGATTTCCTGGGAGGAGGGTTGATGA
CGTCAACGTTCGGGCTCC
>H1_2-H1_188 (SEQ ID NO: 943)
TGGGAAAAAGTGGGGGCTCACGCAGCATTTATAAGGCTCCCATACCTAAAGACATTTCACGGTTATGGTGACTTC
CCCCACAATACATAGCGACATGCAAATATCATGGTCCTTCCGCGGGGCGTGCGGCCTCCCCCTGTCCCGTGCCCC
GTAGGCGTCTTCTCAGCCAGGAGACGCACGCGGCGCGCTGCGTGTTCCCGCCCTGAGTGACTTCTGGGCCGGCGA
TTTCCCTGGGAGGAGGGTTGGATGACGTCAGCATCGCCAACGTTCGGGCTCC
>H1_2-H1_189 (SEQ ID NO: 944)
TGGGAAAAAGTGGGGCTCACGCAGAATTTATAAGGCTCCCATACCTAAAGACATTTCACGGTTATGGTGACTTCC
CCCACAATACATAGCGACATGCAAATATCATGGTCCTTCCGCGGGGCGTGCGGCCTCCCCCTGTCCCGTACCCCG
CAGGCGTCTTCTCAGCCAGGAGGCGCACGCGGCGCGCTGCGCCCTGTTCCCGCCCTGAGTGACTAGGGATTCTGG
GCCCGCGATTTCCCGCTGGGAGGCGGGTTGGATGACGTCAGCATCGCCAGCGTTCGGGCTCC
>H1_2-H1_241 (SEQ ID NO: 945)
TGGGAAAAAGTGGGGGCTCACGCAGCATTTATAAGGCTCCCATACCTAAAGACATTTCACGGTTATGGTGACTTC
CCCACAATACATAGCGACATGCAAATATCGCGGGGCGTGCGGCCTCCCCCTGTCCCGTGTAGGCGTCTTCTCAGC
CAGGACGCACGCGCGCTGCGTGTTCCCGCCCTGAGTGACTTCTGGGCCGGCGATTTCCCTGGGAGGAGGGTTGAT
GACGTCATCGCCAACGTTCGGGCTCC
>H1_2-H1_301 (SEQ ID NO: 946)
TGGGAAAAAGTGGGGCTCACGCAGAATTTATAAGGCTCCCATACCTAAAGACATTTCACGGTTATGGTGACTTCC
CCCACAATACATAGCGACATGCAAATATCATGGTCCTTCCGCGGGGCGTGCGGCCTCCCCCTGTCCCGTACCCCG
CAGGCGTCTTCTCAGCCAGGAGGCGCACGCGGCGCGCTGCGCCCTGTTCCCGCCCTGAGTGACTAGGGATTCTGG
GCCCGCGATTTCCCTGGGAGGCGGGTTGGATGACGTCAGCATCGCCAGCGTTCGGGCTCC
>H1_2-H1_306 (SEQ ID NO: 947)
TGGGAAAAAGTGGGGGCTCACGCAGAATTTATAAGGCTCCCATACCTAAAGACATTTCACGGTTATGGTGACTTC
CCCCACAATACATAGCGACATGCAAATATCATGGTCCTTCCGCGGGGCGTGCGGCCTCCCCCTGTCCCGTACCCC
GTAGGCGTCTTCTCAGCCAGGAGACGCACGCGGCGCGCTGCGCCCTGTTCCCGCCCTGAGTGACTAGGGATTCTG
GGCCGGCGATTTCCCTGGGAGGCGGGTTGGATGACGTCAGCATCGCCAGCGTTCGGGCTCC
>H1_2-H1_312 (SEQ ID NO: 948)
TGGGGAAAGGTGGGCTCAAGCAGAATTTATAAGGCTCCCAAAACTAAAGACATTTTTCGGTTATGGTGACTTCCC
CCACAATACACAGCGACATGCAAATATCATGGCCCTTCCGTGGAGTGTGCCCTCCCTGCGCTCGTCCCCCGGGCC
TCTTCTCAGCCAGGAGGCGCACGGCGCGCTGCGCCTGTTCCCGCCCTGGGGACTAGGAGCGCGCCCGCGGTTCCC
GCTGGGAGGCGGGTTGGATGACGTCAGCATCGCCAGCGTTCGGACTCC
>H1_2-H1_352 (SEQ ID NO: 949)
TGGGGAGTGGGGGGCTCAGGCCGAATTTATAAGGCTCCCAAAACGGAAGACATTTTTCAGTTATGGTGACTTCCC
CCACAAGACACAGCGCTATGCAAATATCATGGCCCCTCCGTGGAGTGTGCCCTCCCCGGCCGCTTCTCAGCCAGG
AAGCGCACGGCGCGTCTGCGCCTGTTTCCCGCCCTGGGGACTAGAAAAGCGCCCGCGCATCCCGGCCGGGCCGCG
GGTTGATGACGTCAGCATCGCCAGCGCTCGAGCGCC
>H1_2-H1_370 (SEQ ID NO: 950)
TGGGGAAAGGTGGGCTCAAGCAGAATTTATAAGGCTCCCAAACCTAAAGACATTTTACGGTTATGGTGACTTCCC
CCACAACACACAGCGACATGCAAATATCATGGTCCTTCCGTGGAGTGTGCCCTCCCTGCGCTCGTCCCCCGGGCC
TCTTCTCAGCCAGGAGGCGCACGCGCGCACGCGCGCTGCGCCTGTTCCCGCCCTGGTGACTAGGAGCGCGCCCGC
GGTTCCCGCTGGGAGGCGGGTTGGATGACGTCAGCATCGCCAGCGTTTGGACTCC
>H1_2-H1_398 (SEQ ID NO: 951)
TGGGAAAAAGTGGGGCTCAAGCAGAATTTATAAGGCTCCCAAACCTAAAGACATTTTACGGTTATGGTGACTTCC
CCCACAACACACAGCGACATGCAAATATCATGGTCCTTCCGCGGGGTGTGCGGCCTCCCTGCTCTCGTCCCCCAG
GCGTCTTCTCAGCCAGGAGGCGCACGCGCGCACGCGCGCTGCGCCCTGTTCCCGCCCTGGTGACTAGGGAGCCTG
AGCCCGCGATTTCCCGCTGGGAGGCGGGTTGGATGACGTCAGCATCGCCAGCGTTTGGACTCC
>H1_2-H1_401 (SEQ ID NO: 952)
TGGGGAGTGGGGGGCTCAGGCCGAATTTATAAGGCTCCCAAAACGGAAGACATTTTTCAGTTATGGTGACTTCCC
CCACAAGACACAGCGCTATGCAAATATCATGGCCCCTCCGTGGAGTGTGCCCTGGCCCCGGCCGCTTCTCAGCCA
GGAAGCGCACGGCGCGCTGCGCCTGTTCCCGCCCTGGGGACTAGAAAAGCGCCCGCGCATCCCGCCGGGCCGCGG
GTTGGATGACGTCAGCATCGCCAGCGCTCGAGCGCC
>H1_2-H1_402 (SEQ ID NO: 953)
TGGGGAGTGGCGGCCTCAGGCGGGATTTATAAGGCTCCCAAAACCGGTGCCATTTCTCAGTGAGGGTGACTTCCC
CCACAATACACAGCGGTATGCAAATATCAGTTGCGTCAGAGTAGAGCGCGGCCTCCCCGGCCTCTCCTCAGCCAG
GAAGCGCGCGGCGCTCCTGTTTTCGTCTCCCGCCCCGGTGACGAGAGACGCGCGCGCGCACCGTAGCCGGGCCGC
GGGTTGGTGACGTAAGCGGCATCCGCTTTCGAGCGCC
>H1_14-H1_18 (SEQ ID NO: 954)
CGGCAAATAACGCGCGCAAACAGCATTTATAATGAGCTCATACCTAAAGCCACTTTACGGTTACGGTGACTTCCC
ACAAGACATTGCGGCATGCAAATATTTTAGTGCGTCCCGCCCCTGGTAGTTCCACGCTAGGACGCACACGCACTA
CGGTTCCCGCCTTTAGACTGCGCTGGCGATTCCAGCGAGCGGACTGATGACGTCAGCGTTGGGGCTCC
>H1_16-H1_17 (SEQ ID NO: 955)
CGGCGAACAACGCGCGCAAACAGCATTTATAATGCGCTCATACCTAAAGCCACTTTTCGGTTACGGTGACTTCCC
ACAAGACATTGCGGCATGCAAATATTTTAGTGCGTCCCGCCCCTGGTAGTTCCACGCTAGGACGCACACGCACTA
CGGTTCCCGCCTTTAGACTGCGCTGGCGATTCCAGCGAGCGGACTGATGACGTCAGCGTTGGGGCTCC
>H1_21-H1_27 (SEQ ID NO: 956)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTC
CGGTTCCCGCCTTTAGACTGCGCTGGCGATTCCAGGGAGCGGGCTGATGACGTCAGTGTTCGGGCTCC
>H1_23-H1_21 (SEQ ID NO: 957)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTC
CGGTTCCCGCCTTTAGACTGCGCTGGCGATTCCAGGGAGCGGACTGATGACGTCAGTGTTCGGGCTCC
>H1_23-H1_24 (SEQ ID NO: 958)
CGGCCAACAGCTCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTG
CGGTTCCCGCCTTTAGACTGCGCTGGCGATTCCAGGGAGCGGACTGATGACGTCAGTGTTCGGGCTCC
>H1_25-H1_26 (SEQ ID NO: 959)
CGGCAAACAATGCGCGCAAACAGCATTTATAATGAGCTCATACCTAAAGCCACTTTACGGTTACGGTGACTTCCC
ACAAGACATTGCGATATGTAAATATTTTAGTGCATCCCGCCCCTGGTAGTTCCACGCTAGGACGCACACGCACTA
CGGTTCCCGCCTTTAGATTGCGCTGGCGATTCCAGGGAGCGGACTGATGACGTCAGCGTTCGGGCTCC
>H1_27-H1_28 (SEQ ID NO: 960)
CGGCAAACAACGCGCGCAAACAGCATTTATAATGAGCTCATACCTAAAGCAACTTTTCGGTTACGGTGACTTCCC
ACAAGCCATTGCGACATGCAAATATTTTAGTGTATCCCGCCCCTGGTAGTTCCACGCTAGGACGCACACGCACTC
CGGTTCCCGCCTTTAGACTGCGCTGGCGATTCCGGGGAGCGGGCTGATGACGTCAGTGTTCGGGCTCC
>H1_31-H1_33 (SEQ ID NO: 961)
CGGCAAACAATGCGTGCACACAGCACTTATAATGCGCTCACACCTAAAGCCACTTTTCAGTTACGGTGACTTCCC
ACAAGACATTGCGATATGCAAATATTTTAGCGCATCCCGCCCCTGGTAGTTCCACGCGAGGACGCACACGCACTA
CGGTTCCCGCCTTTAGACTGCGCTGGCGATTCCAGGGAGCGGCCTGATGACGTCAGCGTTCGGGCTCC
>H1_34-H1_32 (SEQ ID NO: 962)
CGGCAAACAATGCGTGCACACAGCATTTATAATGCGCTCACACCTAAAGCCACTTTTCAGTTACGGTGACTTCCC
ACAAGACATTGCGATATGCAAATATTTTAGCGCGTCCCGCCCCTGGTAGTTCCACGCGAGGACGCACACGCACTA
CGGTTCCCGCCTTTAGACTGCGCTGGCGATTCCAGGGAGCGGCCTGATGACGTCAGCGTTCGGGCTCC
>H1_35-H1 37 (SEQ ID NO: 963)
CGGCAAACAGTGCGCGCAAACAGCATTTATAATGCGCTCATACCTAAAGCCACTTTTCGGTTACGGTGACTTCCC
ACAAGACATTGCGACATGCAAATATTTTAGTGCGTCCCGCCCCTGGTAGTTCCACGCTAGGACGCACACGCACTA
CGGTTCCCGCCTTTAGACTGCGCTGGCGATTCCTGGGAGCGGACTGATGACGTCAGCGTTCGGGCTCC
>H1_36-H1_20 (SEQ ID NO: 964)
CGGCAAACAACGCGCGCAAACAGCATTTATAATGCGCTCATACCTAAAGCCACTTTACGGTTACGGTGACTTCCC
ACAAGACATTGCGACATGCAAATATTTTAGTGCATCCCGCCCCTGGTAGTTCCACGCTAGGACGCACACGCACTA
CGGTTCCCGCCTTTAGACTGCGCTGGCGATTCCAGGGAGCGGACTGATGACGTCAGCGTTCGGGCTCC
>H1_39-H1_22 (SEQ ID NO: 965)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTA
CGGTTCCCGCCTTTAGACTGCGCTGGCGATTCCAGGGAGCGGACTGATGACGTCAGTGTTCGGGCTCC
>H1_39-H1_89 (SEQ ID NO: 966)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTA
CGGTTCCCGCCTTTAGACTGCGCTGGCGATTCCAGGGAGCGGGCTGATGACGTCAGCGCCCGGGCTCC
>H1_41-H1_40 (SEQ ID NO: 967)
TGGCAAACAATCCGCGCAAACAGCATTTATAATGCGCTCATACCTAGAGCCACTTTTCGGTTACGGTGACTTCTC
ACAAGACATTGGGACATGCAAATATTACAGTGCGTCCCGCCCCTGGTTAGTTCTACGCTAGGACGCACACGCACT
ACGGTTCCCGCCTTTAGACTGCGCTGGCGGTTCCTGGGAGCGGACTGATGACGTCAGTGTTCGGGATCC
>H1_41-H1_55 (SEQ ID NO: 968)
TGGCAAACAACGCGCGCAAACAGCATTTATAATGCGCTCATACCTAGAGCCACTTTTCGGTTACGGTGACTTCTC
ACAAGACATTGGGACATGCAAATATTACAGTGCGTCCCGCCCCTGGTTAGTTCTACGCTAGGACGCACACGCACT
ACGGTTCCCGCCTTTAGACTGCGCTGGCGATTCCTGGGAGCGGACTGATGACGTCAGCGTTCGGGATCC
>H1_47-H1_41 (SEQ ID NO: 969)
TGGCAAACAACGCCGGCGCAAACAGCATTTATAATGCGCTCATACCTAGAGCCACTTTTCGGTTACGGTGACTTC
TCAACAAGACATTGGGACATGCAAATATTACAGTGCGTCCCGCCCCTGGTGTAGTTCTACGCTAGGACGCACACG
CACTACGGTTCCCGCCTTTAGACGACTGCGCTGGCGATTCCTGGGAGCGGACTGATGACGTCAGCGTTCGGGATC
C
>H1_47-H1_43 (SEQ ID NO: 970)
TGGCAAACACCGCACGCAAATAGCATTTATAATGTGCTCATACCTAGAGCCACTTTTCGGTTACGGTGACTTCTC
AAAAAGACAGTGGGACATGCAAATATTACAGTGCGTCCCGCCCCTGGTAGGTCTACGCTAGGACGCACGCGCACT
ACGGTTCCCGCCTATAGACTGCGCTGGCGATTCCTGGGAGCGGACTGATGACGTCAGCGTTCGGGATCC
>H1_47-H1_51 (SEQ ID NO: 971)
TGGCAAACAACGCCGGCGCAAACAGCATTTATAATGTGCTCATACCTAGAGCCACTTTTCGGTTACGGTGACTTC
TCAACAAGACATTGGGACATGCAAATATTACAGTGCGTCCCGCCCCTGGTGTAGTTCTACGCTAGGACGCACGCG
CACTACGGTTCCCGCCTATAGACGACTGCGCTGGCGATTCCTGGGAGCGGACTGATGACGTCAGCGTTCGGGATC
C
>H1_47-H1_94 (SEQ ID NO: 972)
TGGCAAACAACGCCGGCGCAAACAGCATTTATAATGTGCTCATACCTAGAGCCACTTTTCGGTTACGGTGACTTC
TCAAAAAGACATTGGGACATGCAAATATTACAGTGCGTCCCGCCCCTGGTGTAGTTCTACGCTAGGACGCACGCG
CACTACGGTTCCCGCCTATAGACGACTGCGCTGGCGATTCCTGGGAGCGGACTGATGACGTCAGCGTTCGGGATC
C
>H1_53-H1_57 (SEQ ID NO: 973)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTGCGCTAGGACGCAGACGCACTA
CGGTTCCCGCCTTTAGACCGCGCTGGCGATTCCTGGGAGCGGACTGATGACGTCAGCGTTCGGGATCC
>H1_59-H1_54 (SEQ ID NO: 974)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTGCGCTAGGACGCAGACGCACTA
CGGTTCCCGCCTTTAGACTGCGCTGGCGATTCCTGGGAGCGGACTGATGACGTCAGCGTTCGGGATCC
>H1_59-H1_60 (SEQ ID NO: 975)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTACGGACGCAGACGCACTACGGT
TCCCGCCTTTAGACTGCGCTGGCGATTCCTGGGAGCGGACTGATGACGTCAGCGTTCGGGATCC
>H1_61-H1_62 (SEQ ID NO: 976)
TGGCAAACACCGCGCGCAACCAGCATTTATAATGCGCTCGTACCTAAAGGCACTTGTCGGTTACGGTGACTTCCC
ACAAGACATTGCGACATGCAAATACTACAGTGCGTCCCGCCCCTGGTAGTTCCACGCTGGGACGCACACGCAGTA
CGGTTCCCGCCTTTAGACTGCGCTGGCGATTCCTGGGAGAGGATTGATGACGTCAGCGTTCGGGCTCC
>H1_63-H1_64 (SEQ ID NO: 977)
CGGCACAAAACGCGGGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACACACACGCACTA
TGCTTCCGGCCTTTAGACTGCGCCGGTGATTCCAGGGAGCGGACTGATGACGTCAGCGTTCCGGCTCC
>H1_65-H1_63 (SEQ ID NO: 978)
CGGCAAAAAACGCGGGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACACACACGCACTA
TGGTTCCGGCCTTTAGACTGCGCCGGTGATTCCAGGGAGCGGACTGATGACGTCAGCGTTCGGGCTCC
>H1_66-H1_65 (SEQ ID NO: 979)
CGGCAAACAACGCGCGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACGCACACGCACTA
CGGTTCCCGCCTTTAGACTGCGCTGGCGATTCCAGGGAGCGGACTGATGACGTCAGCGTTCGGGCTCC
>H1_67-H1_69 (SEQ ID NO: 980)
TGGCGAATAACACGCGCAAAGAGCATTTATAACGCGCTCAGACCTAAAGCCACTTGTCGCTTACGGTGACTTCCC
ATAAGACATTGCAATATGCAAATACTCCAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_70-H1_71 (SEQ ID NO: 981)
TGGCGAAAATCACGCGCAAAGAGCATTTATAACGTGCTCAGACCTAAAGCCACTTGTCGCTTACGGTGACTCCCC
ATAAGACATTGCGATATGCAAATACTGCAGTGCGTCCCGCCCCTGGCAGTTCCACGCTGGGACGTACACGTACTA
CGGTTCCCGCCTTTAGACTGCGCTCGCGATTCCTGGGAGAGGGCTGATGACGTCAGCGTTCGGGCTCC
>H1_70-H1_76 (SEQ ID NO: 982)
TGGCGAAAAACACGCGCAAAGAGCATTTATAACGTGCTCAGACCTAAAGCCACTTGTCGCTTACGGTGACTCCCC
ATAAGACATTGCGATATGCAAATACTGCAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_77-H1_79 (SEQ ID NO: 983)
CGGCGAAAAACACGCGCAAAGAGCGTTTATAATGCGCTCAGACCTAAAGTAACTTGTCACTTACGGTGACTTCCC
ATAAGACATTGCGATATGCAAATATTCCAGTGCGTCCCGCCCCTGGCAGTTCCACGCCGGGACGTGCACGCACTA
CGGTTCCCGCCTTTAGACTGCGCTGGCGATTCCTGGGAGAGGGCTGATGACGTCAGCGTGCGGGCTCC
>H1_77-H1_80 (SEQ ID NO: 984)
CGGCGAAAAACACGCGCAAAGAGCGTTTATAACGCGCTCAGACCTAAAGCTACTTGTCACTTACGGTGACTTCCC
ATAAGACATTGCGATATGCAAATATTCCAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTGCGGGCTCC
>H1_77-H1_81 (SEQ ID NO: 985)
CGGCGAAAAACACGCGCAAAGAGCGTTTATAACGCGCTCAGACCTAAAGCTACTTGTCACTTACGGTGACTTCCC
ATAAGACATTGCGATATGCAAATATTCCAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_77-H1_82 (SEQ ID NO: 986)
TGGCGAAAAACACGCGCAAAGAGCATTTATAACGCGCTCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_82-H1_67 (SEQ ID NO: 987)
TGGCGAAAAACACGCGCAAAGAGCATTTATAACGCGCTCAGACCTAAAGCCACTTGTCGCTTACGGTGACTTCCC
ATAAGACATTGCGATATGCAAATACTACAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_83-H1_77 (SEQ ID NO: 988)
TGGCGAAAAACGCGCGCAAAGAGCATTTATAATGCGCTCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_83-H1_87 (SEQ ID NO: 989)
TGGAGGAGAACGCGCGCAAAGAGCATTTATAATGCGCGCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCGCTA
CGGTTCCCGCCTTTAGACTGCGCTGGCGATTCCTGGGAGAGGGCTGATGACGTCAGCATTCGGGCTCC
>H1_95-H1_140 (SEQ ID NO: 990)
TGGCAAAAACTGAGCTCAAGCAGCATTTATAAGGCTCCCAAACCTAAAGACATTTGTCGGTTATGGTGACTTCCC
ACAACACATTGCGACATGCAAATACTGCGGAGCGTACCCCCTCCCCTGGCAACTCCTCGCTGGGACGCACGCGCG
CTACGTGTTCCCGCCTTTTGACTGCGCCGGCGATACCTGGGAGAGGGTTGATGACGTCAGCGTTCGGGCTCC
>H1_98-H1_100 (SEQ ID NO: 991)
TGGGAAAGGGTGGGCTCACGCAGCCTTTATAAGGCTCCCAAACTTAAAGACATTTCTCGGTTATGGCGACTTCCC
ACAAGACATAGCGACATGCAAATACTGCAGACCTGTGGCGCCGACCCGGTCCTGTGCAGCCATCTTTACGGCTGG
GACGCACGCGCGCTGCGTGTTCCCGCCCTGTGACTGCGCCGGCGATTACTGGGAGAGGATTGATGACGTCAACGT
TCGGGTTCC
>H1_100-H1_101 (SEQ ID NO: 992)
TGAGAGAGGGTGGGCTCACGCCACCTTTATAAGGCTCCCAAACTTAAAGACATTTCTCGGTTATGGCGACTTCCC
ACAACACATAGCGACATGCAAATACTGCAGACCTGTGGCGCCGACCCGGTCCTGTGCAGCCATCTTTACGGCTGG
GACGCACGCGCGCTGCGTGTTCCCGCCCTGTGACTGCGCCGGCGATTACTGGGAGAGGATTGATGACGTCAACGT
TCGGGTTCC
>H1_109-H1_107 (SEQ ID NO: 993)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCAATACATGTTCCCGCCTTGAGACTGCGCCGGCAGATTCCTAGGAAGTGGTTGATGACGTCGATGTTAGGG
ATCC
>H1_111-H1_109 (SEQ ID NO: 994)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCAATACATGTTCCCGCCTTGAGACTGCGCCGGCCGATTCCTAGGAAGTGGTTGATGACGTCGATGTTGGGG
CTCC
>H1_112-H1_111 (SEQ ID NO: 995)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCACTACATGTTCCCGCCTTGAGACTGCGCCGGCCGATTCCTAGGAAGTGGTTGATGACGTCGATGTTGGGG
CTCC
>H1_113-H1_112 (SEQ ID NO: 996)
CGGAGAAAACCTGCTTCACCGAGCATTTATAAAGCTCCCATACTTAAAGAGATTTCATAGTTATGGTGACTTCCC
ACAAGACATTGCGACATGCAAATATTGTGGAGCGTACTTCCCCGTCCTGTGCAGGCAGCTTCCCGCCAGGACGCA
CGCGCGCTGCGTGTTCCCGCCTTGAGACTGCGCCGGCGATTTCCTAGGAGGGTGGTTGATGACGTCAATGTTCGG
GCTCC
>H1_114-H1_121 (SEQ ID NO: 997)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGCGTACCTCCCCTGGCAACTCCTCGCTGGGACGCACGCGCGCTG
CGTGCTCCCGCCTTTTGACTGCGCCGGCGATACTTGGGAGAGGGTTGATGACGTCAGCGTTCTGGCTCC
>H1_117-H1_115 (SEQ ID NO: 998)
TGCCGAAAGTTTAGCTCAACCTGCATTTATAAAGCTCCCAAACCTAAAGACATTTGTCGGTTATGGTGACTTCCC
GCAACACATTGCGACATGCAAATACTGCGGAGTGCACCTCCCCTGGCAACTCCTCGCTGGGACGCACGCGCGCTG
CGTGCTCCCGCCTTTTGACTGCGCCGGCGATACTTGGGAGAGGGTTGATGACGTCAGCGTTCTGGCTCC
>H1_118-H1_114 (SEQ ID NO: 999)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTGCGTGCTCCCGCCTTTTGACTGCGCCGGCGATACTTGGGAGAGGGTTGATGACGTCAGCGTTCTGGCTCC
>H1_118-H1_122 (SEQ ID NO: 1000)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGTGCACCCCCTCCCCTGGCAACTCCTCGCTGGGACGCACGCGCG
CTGCGTGCTCCCGCCTTTTGACTGCGCCGGCGATACTTGGGAGAGGGTTGATGACGTCAGCGTTCTGGCTCC
>H1_118-H1_123 (SEQ ID NO: 1001)
TGCCGAAAATTTAGCTCAAGCCGCATTTATAAGGCTCCCAAACCTAAATACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_124-H1_126 (SEQ ID NO: 1002)
CGCCGAAAACCAGGCTCAAGCCACATTTATAAGGCTCCCAAAGCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGAGTTGATGACGTCAGCGTTCTGGCTCC
>H1_124-H1_129 (SEQ ID NO: 1003)
CGCCGAAAACCAGGCTCAAGCCACATTTATAAGGCTCCCAAACCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGGGTTGATGACGTCAGCGTTCTGGCTCC
>H1_129-H1_127 (SEQ ID NO: 1004)
CGCCGAAAACCAGGCTCAAGCCACATTTATAAGGCTCGCAAACCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGGGTTGATGACGTCAGCGTTCTGGCTCC
>H1_133-H1_132 (SEQ ID NO: 1005)
CGCCGAAAACCAGGCTCAAGCCACATTTATAAGGCTCCCAAACCTAAGTACATTTGTCGGTTATGGTGACTTCCC
GCAACACATTGCGACATGCAAATACTGCGGAGCGTCCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_134-H1_133 (SEQ ID NO: 1006)
CGCCGAAAACCAGGCTCAAGCCACATTTATAAGGCTCCCAAACCTAAGTACATTTGTCGGTTATGGTGACTTCCC
GCAACACATTGCGACATGCAAATACTGCGGAGCGTTCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_135-H1_134 (SEQ ID NO: 1007)
CGCCGAAAACCAGGCTCAAGCCACATTTATAAGGCTCCCAAACCTAAATACATTTGTCGGTTATGGTGACTTCCC
GCAACACATTGCGACATGCAAATACTGCGGAGCGTTCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_136-H1_137 (SEQ ID NO: 1008)
TGCCGAAAACCTAGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_137-H1_124 (SEQ ID NO: 1009)
CGCCGAAAACCAGGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_137-H1_138 (SEQ ID NO: 1100)
CGCCGAAAGCCAGGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGACACTTGGGAGAGGGTTGATGACGTCAGCGTTCTGGCTCC
>H1_140-H1_141 (SEQ ID NO: 1101)
TGGCAAAAACTGAGCTCAAGCCGCATTTATAAGGCTCCCAAACCTAAAGACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_141-H1_118 (SEQ ID NO: 1102)
TGCCGAAAACTTAGCTCAAGCCGCATTTATAAGGCTCCCAAACCTAAATACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_141-H1_139 (SEQ ID NO: 1103)
TGCCGAAAACTTAGCTCACGCCGCACTTATAAGGCTCCCAAACCTAAATACATTTGTAGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGAGCCGGCGATACTTGGGAGAGGGTTGATGACGTCAGCGTTCTGGCTCC
>H1_141-H1_142 (SEQ ID NO: 1104)
TGCCGAAAGCTTACCTTCGCCCGCCTTATAAGGCTCCCAAACCTAAATACATTTGTAGGTTATGGTGACTTCCCG
CAACACATTGCGACATGCAAATACTGCGGAGCGTACCTCCCCTGGAAACTCCTCGCTGGGACGCACGCGCGTTAC
GTGCTCCCGCCTTTTGACTGAGCCGGCGATACTTGGGAGAGGGTTGATGACGTCAGCGTTCTGGCTCC
>H1_150-H1_146 (SEQ ID NO: 1105)
TGGGAAAGGGTGGCCCCGCCGAGCATTTATAAGACTCCCATACCTAAAGACATTTCTCAGTTATGGTGATTTCCC
TACAACACACAGCAACATGCAAATATCGAGGGGTGTACCGCCCCTGTCCTTTGTAGACGTCTTCTCTCCAGGACG
CACGCGCGCTGTATTCCCGCCTTGTGACTCTAGGCGGGCGATTCCTGGGAGAGGGTTGATGACGTCCAAGTTCTG
GCTTC
>H1_151-H1_150 (SEQ ID NO: 1106)
TGGGAAAGGGTGGCCCCGCCGAGCATTTATAAGACTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCTCCAGGACG
CACGCGCGCTGTATTCCCGCCTTGTGACTCTAGGCGGGCGATTCCTGGGAGAGGGTTGATGACGTCCAAGTTCTG
GCTTC
>H1_151-H1_153 (SEQ ID NO: 1107)
TGGGAAAGGGTGGCTCCGCCGAGCATTTATAAGACTCCCATACCTAAAGACATTTCTCAGTTATGGTGACTTCCC
ACAACGCACAGCAACATGCAAATATCGAGGGGTGTACCGCCCCTGTCCTTTGTAGACGTCTTCTCTCCAGGACGC
ACGCGCGCTGTATTCCCGCCTTGTGACTAGGCGGGCGATTCCTGGGAGAGGGTTGATGACGCCCAAGTTCTGGCT
TC
>H1_151-H1_155 (SEQ ID NO: 1108)
TGGGAAAGGGTGGCCCCGCCGAGCATTTATAAGACTCCCATACCTAAAGACATTTCTCAGTTATGGTGACTTCCC
ACAACACACAGCAACATGCAAATATCGAGGGGTGTACCGCCCCTGTCCTTTGTAGACGTCTTCTCTCCAGGACGC
ACGCGCGCTGTATTCCCGCCTTGTGACTAGGCGGGCGATTCCTGGGAGAGGGTTGATGACGTCCAAGTTCTGGCT
TC
>H1_157-H1_156 (SEQ ID NO: 1109)
TGGGAAAGGGGGGCTCCGCTGAGCGTTTATAAGGCTCCCATACCTAAAGACATTTCACAGTTATGGTGACTTCCC
ACAACACACAGCAACATGCAAATACAGAGGGGTGTACCGCCCCTGTCCTTTGTAGACGTCTTCTCGCCAGGACGC
ACGCGCGCTGTGTTCCCGCCTTGTGACTCTAGGCGGGCGATTCCTGGGAGAGGGTTGATGACGTCCAAGTTCTGA
CTCC
>H1_157-H1_158 (SEQ ID NO: 1110)
TGGGAGAGGGAGGTTCCGCTGAGCGTTTATAAGGCTCCCATATCTAAAGACATTTCACAGTTATGGTGACTTCCC
ACAACACACAGCAACATGCAAATACAGAGAAGCGTACCACCCCTGTCCTTTGCAGACGTCTTCTAGCCAGGACGC
ACGCGCACTGTGTTCCCGCCTTGTGACTCGAGGCGGGCGATACCTGGGAGAGGGTTGATGACGTCCAAGTTCTGA
CTCC
>H1_157-H1_160 (SEQ ID NO: 1111)
TGGGAAAGGGTGGCTCCGCCGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCGCCAGGACG
CACGCGCGCTGTGTTCCCGCCTTGTGACTCTAGGCGGGCGATTCCTGGGAGAGGGTTGATGACGTCCAAGTTCTG
ACTCC
>H1_160-H1_151 (SEQ ID NO: 1112)
TGGGAAAGGGTGGCTCCGCCGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCTCCAGGACG
CACGCGCGCTGTGTTCCCGCCTTGTGACTCTAGGCGGGCGATTCCTGGGAGAGGGTTGATGACGTCCAAGTTCTG
ACTCC
>H1_160-H1_159 (SEQ ID NO: 1113)
CAGGCAAAAGCAGTTCGGCCGAGAATTTATAAGGCTCCAATACCTAAAGACATTTCTCAGTTACGGTGACTTCCC
ACAACACACAGCAACATGCAAATATCGAGAGGTGTACCGCCCCTGTCCTTTGTAGACGTCTTCTCTTCGGGACGC
ACGCGCGCTGTGTTCCCGCCTTATGACTCTAGGCGGGCGATTCCTGGGAGAGGGTTGATGACGTCCAAGTTCTGA
CTCC
>H1_160-H1_161 (SEQ ID NO: 1114)
CAGGCAAAAGCAATTCGGCCGAGAATTTATAAGGCTCCAATACCTAAAGACATTTCTCAGTTACGGTGACTTCCC
ACAACACACAGCAACATGCAAATATCGAGAGGTGTACCGCCCCTGTCCTTTGTAGACGTCTTCTCTTCGGGACGC
ACGCGCGCTGTGTTCCCGCCTTATGACTCTAGGCGGGCGATTCCTGGGAGAGGGTTGATGACGTCCAAGTTCTGA
CTCC
>H1_162-H1_157 (SEQ ID NO: 1115)
TGGGAAAAGGTGGCTCCACAGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCGCCAGGACG
CACGCGCGCTGTGTTCCCGCCTTGTGACTCTAGGCGGGCGATTCCTGGGAGAGGGTTGATGACGTCCAAGTTCTG
ACTCC
>H1_163-H1_196 (SEQ ID NO: 1116)
TGGGAAAGGGTGGCCCCACAGAGCATTTATAAGGCTCCCATACCTAAAGACATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCCCGGGAGAGGGTTGCTGACGGGAACGTTCAG
GCTCC
>H1_164-H1_167 (SEQ ID NO: 1117)
TGGGAAAGGGTGGTCCTGAGGCGGATTTATAAGGCTCCCACATCTAAAGGCATTTCACAGTCATGGTGACTTCCC
ACAATACATAGCAACATGCAAATTTCGCGGGGAGTACCTCCCCTGTCCCTTGTACGCGGCTTCTCAGGACGCACG
CACGCGCTCTGTGTTCCCGCCCTGTGACTCTAGGAGGGCAATTCCTGGGACAGTGTTCTGACGGGAACGTTCAGG
CTCC
>H1_166-H1_164 (SEQ ID NO: 1118)
TGGGAAAGGGTGGTCCTGAGGCGGATTTATAAGGCTCCCATATCTAAAGGCATTTCACAGTCATGGTGACTTCCC
ACAATACATAGCAACATGCAAATTTCGCGGGGAGTACCTCCCCTGTCCCTTGTACGCGGCTTCTCAGGACGCACG
CACGCGCTCTGTGTTCCCGCCCTGTGACTCTAGGAGGGCAATTCCTGGGACAGTGTTCTGACGGGAACGTTCAGG
CTCC
>H1_169-H1_165 (SEQ ID NO: 1119)
TGGGAAAAGGTGGTCCTGGGGCGGATTTATAAGGCTCCCATATCTAAAGGCATTTCACAGTCATGGTGACTTCCC
ACAATACATAGCAACATGCAAATATCGCGGGGAGTACCTCCCCTGTCCCTTGTACGCGGCTTCTCAGGACGCACG
CACGCGCTCTGTGTTCCCGCCCTGTGACTCTAGGCGGGCAATTCCTGGGACAGTGTTCTGACGGGAACGTTCAGG
CTCC
>H1_171-H1_172 (SEQ ID NO: 1120)
TGGAAAAGAGTGGCCCCAGAGAGCATTTATAAGGCTCCCGCACCTAAATGCATTTATCAGTTATGGTGACTTCCC
ACAATACATAGCAACATGCAAATATAGCGGGGAGTACCTCCCCTGTCCCTTGTCCGTGTCTTCTCAGGACGCACG
CACGCGCGCTGTGTTCCCGCCCTGTGACTCTAAGCGGGCAATTCCTGGAAGAGGCTTGCTGACGGGAACGTTCAG
GCTCC
>H1_171-H1_173 (SEQ ID NO: 1121)
TGGGAAAGAGTGGCCCCAGAGAGCATTTATAAGGCTCCCGCACCTAAATGCATTTATCAGTTATGGTGACTTCCC
ACAATACATAGCAACATGCAAATATAGCGGGGAGTACCTCCCCTGTCCCTTGTACGTGTCTTCTCAGGACGCACG
CACGCGCGCTGTGTTCCCGCCCTGTGACTCTAAGCGGGCAATTCCTGGAAGAGGCTTGCTGACGGGAACGTTCAG
GCTCC
>H1_175-H1_176 (SEQ ID NO: 1122)
TGGGAAAGGGTGGCCCCAGAGAGCATTTATAAGGCTCCCGCACCTAAATGCATTTATCAGTTATGGTGACTTCCC
ACAATACATAGCAACATGTAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCAG
GCTCC
>H1_177-H1_171 (SEQ ID NO: 1123)
TGGGAAAGGGTGGCCCCAGAGAGCATTTATAAGGCTCCCGCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCAG
GCTCC
>H1_177-H1_178 (SEQ ID NO: 1124)
TGGGAAACGGTGGCCCCAAAGAGCACTTATAAAGCCCCCTCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGTGGACAATTCCTGGGGGAGGCTTGCTGACGGGAACGTTCCG
GCTCC
>H1_177-H1_406 (SEQ ID NO: 1125)
TGGGAAACGGTGGCCCCAAAGAGCATTTATAAAGCTCCCTCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCCG
GCTCC
>H1_181-H1_182 (SEQ ID NO: 1126)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGCGGGTTTGCTGACAAGAACGTTCAG
GCTCC
>H1_182-H1_183 (SEQ ID NO: 1127)
TGGGAAAGGGTGGGCCCAGCGAGCATTTATAAGGCTCCCGCATCTAAAGACATTTTACAATTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGCGGGTTTGCTGACAAGAACGTTCAG
GCTCC
>H1_184-H1_185 (SEQ ID NO: 1128)
TGGGAAAGGGTGGCCCCAGAGAGCATTTATAAGGCTCCCGCACCTAAAGGCATTTAACAGTTATGGTGACTTCCC
ACAACGCGTAGCAACATGCAAATATCGCAGGGAGTACCGCCCCTGTCCCTTGTACGCATCTTCTCAGGACGCACG
CACGCGCGCTGTGTTCCCGCCCTGTGACTCCAGGCGGGCATTTCCCGGGGCGGGTTTGCTGACAGGAACGTTCAG
GCTCC
>H1_188-H1_162 (SEQ ID NO: 1129)
TGGGAAAAGGTGGCCCCACAGAGCATTTATAAGGCTCCCATACCTAAAGACATTTCTCAGTTATGGTGACTTCCC
TACAATACATAGCAACATGCAAATATCGCGGGGCGTACCTCCCCTGTCCCTTGTAGGCGTCTTCTCAGCCAGGAC
GCACGCGCGCTGTGTTCCCGCCCTGTGACTCTAGGCGGGCGATTCCTGGGAGAGGGTTGATGACGTCAACGTTCG
GGCTCC
>H1_188-H1_163 (SEQ ID NO: 1130)
TGGGAAAGGGTGGCCCCAGAGAGCATTTATAAGGCTCCCATACCTAAAGGCATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCCCGGGAGAGGGTTGCTGACGGGAACGTTCAG
GCTCC
>H1_188-H1_170 (SEQ ID NO: 1131)
TGGGAAAGGGTGGCCCCAGAGAGCATTTATAAGGCTCCCGCACCTAAAGGCATTTTACAGTTATGGTGACTTCCC
ACAACGCGTAGCAACATGCAAATATCGCGGGGAGTACCGCCCCTGTCCCTTGTACGCGTCTTCTCAGGACGCACG
CACGCGCGCTGTGTTCCCGCCCTGTGACTCCAGGCGGGCAATTCCCGGGGCGGGTTTGCTGACGGGAACGTTCAG
GCTCC
>H1_188-H1_177 (SEQ ID NO: 1132)
TGGGAAAGGGTGGCCCCAGAGAGCATTTATAAGGCTCCCATACCTAAAGGCATTTCTCAGTTATGGTGACTTCCC
ACAATACATAGCAACATGCAAATATCGCGGGGAGTACCTCCCCTGTCCCTTGTACGCGTCTTCTCAGGACGCACG
CACGCGCGCTGTGTTCCCGCCCTGTGACTCTAGGCGGGCAATTCCTGGGAGAGGGTTGCTGACGGGAACGTTCAG
GCTCC
>H1_188-H1_179 (SEQ ID NO: 1133)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGGACGCACG
CACGCGCGCTGTGTTCCCGCCCTGTGACTCCAGGCGGGTATTTCCCGGGGCGGGTTTGCTGACAGGAACGTTCAG
GCTCC
>H1_188-H1_180 (SEQ ID NO: 1134)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGCGGGTTTGCTGACAGGAACGTTCAG
GCTCC
>H1_188-H1_186 (SEQ ID NO: 1135)
TGGGAAAGGGTGGCCCCACCGAGCATTTATAAGGCTCCCGCATCTAAAGACATTTTACAGTTATGGTGACTTCCC
ACAACGCGTAGCAACATGCAAATATCGCGGAGAGTACCGCCCCTGTCCCATGCACGCGTCTTCTCAGCACGCACG
CACGCGCGCTGTGTTCCCGCCCTGTGACTCCAGGCGGGTATTTCCAGGGGCGGGTTTGCTGACAGGAACGTTCAG
GCTTC
>H1_188-H1_198 (SEQ ID NO: 1136)
TGGGAAAAGGTGGCCCCAGAGAGCATTTATAAGGCTCCCATACCTAAAGGCATTTCTCAGTTATGGTGACTTCCC
ACAATACATAGCAACATGCAAATATCGCGGGGAGTACCTCCCCTGTCCCTTGTACGCGTCTTCTCAGGACGCACG
CACGCGCGCTGTGTTCCCGCCCTGTGACTCTAGGCGGGCAATTCCTGGGAGAGGGTTGCTGACGGGAACGTTCAG
GCTCC
>H1_188-H1_203 (SEQ ID NO: 1137)
TGGGAAAAAGTGGGGCCTCACGCAGCATTTATAAGGCTCCCATACCTAAAGACATTTCACGGTTATGGTGACTTC
CCCCACAATACATAGCGACATGCAAATATCATGGTCCTTCCGCGGGGCGTGCCTCCCCCTGTCCCTTGGCCCGTA
GGCGTCTTCTCAGCCAGGAGACGCACGCGGCGCGCTGCGTGTTCCCGCCCTGTGACTTCTAGGCGGGCGATTCCC
TGGGAGAGGGTTGGATGACGTCAGCATCGCCAACGTTCGGGCTCC
>H1_189-H1_1 (SEQ ID NO: 1138)
TGGGAAAAGGTGGGCCCACGCAGAATTTATAAGGCTCCCATACCTAAAGACATTTTACGATTATGGTGACTTCCC
ACAATACATAGCGACATGCAAATATCGCGGGGCGTGCCTCCCCTGTCCCGTCACAGGCGTCTTCTCAGCCAGGGC
GCACGCGCGCTGCGTGTTCCCGCCCTGTGACTCTGGGCCCGCGATTCCTGGGAGCGGGTTGATGACGTCAGCGTT
CGGGCTCC
>H1_189-H1_192 (SEQ ID NO: 1139)
TGGGAAAGGGTGGACCCACCGAGCATTTATAAGGCTCCCGCATCTAAAGACATTTTACAGTTATGGTGACTTCCC
ACAACGCGTAGCAACATGCAAATATCGTGGAGAGTACCGCCCCTGTCCCATGCACGCGTCTTCTCAGCACGCACG
CACGCGCGCTGTGTTTCCCGCCCTGTGACTCCAGGCGGGTATTTCCAGGGGCGGGTTTGCTGACAGGAACGTTCA
GGCTTC
>H1_189-H1_227 (SEQ ID NO: 1140)
TGGGAAAAGGTGGGCCCACGCAGAATTTATAAGGCTCCCATACCTAAAGACATTTCACGATTATGGTGACTTCCC
ACAATACATAGCGACATGCAAATATCGCGGGGCGTGCCTCCCCCTGTCCCGTACCCCACAGGCGTCTTCTCAGCC
AGGGCGCACGCGCGCTGCGTGTTCCCGCCCTGAGTGACTAGGGATTCTGGGCCCGCGATTCCCGTGGGAGCGGGT
TGATGACGTCAGCGTTCGGGCTCC
>H1_189-H1_234 (SEQ ID NO: 1141)
TGGGAAAAGGTGGGCCCACGCAGAATTTATAAGGCTCCCATACCTAAAGACATTTCACGATTATGGTGACTTCCC
ACAATACATAGCGACATGCAAATATCGCGGGGCGTGCCTCCCCTGTCCCGTACCCCACAGGCGTCTTCTCAGCCA
GGGCGCACGCGCGCTGCGTGTTCCCGCCCTGAGTGACTAGGGATTCTGGGCCCGCGATTCCCGTGGGAGCGGGTT
GATGACGTCAGCGTTCGGGCTCC
>H1_189-H1_237 (SEQ ID NO: 1142)
TGGGAAAAGGTGGGCCCACGCAGAATTTATAAGGCTCCCATACCTAAAGACATTTTACGATTATGGTGACTTCCC
ACAATACATAGCGACATGCAAATATCGCGGGGCGTGCCTCCCCTGTCCCGTCACAGGCGTCTTCTCAGCCAGGGC
GCACGCGCGCTGCGTGTTCCCGCCCTGAGTGACTCTGGGCCCGCGATTCCCGTGGGAGCGGGTTGATGACGTCAG
CGTTCGGGCTCC
>H1_189-H1_286 (SEQ ID NO: 1143)
TGGGAAAAGGTGGGCCCACGGAGAATTTATAAGGCTCCCATACCTAAAGACATTTTACGATTATGGTGACTTCCC
ACAACACATAGCGACATGCAAATATCGCGGGGCGTGCCTCCCCTGTCCCGTACAGGCGTCTTCTCAGCCAGGGCG
CACGCGCGCTGCGTGTTCCCGCCCTGTGACTCCGGGCCCGCGATTCCTGGGAGCGGGTTGATGACGTCAGCGTTC
GGGCTCC
>H1_195-H1_184 (SEQ ID NO: 1144)
TGGGAAAGGGTGGCCCCAGAGAGCATTTATAAGGCTCCCGCACCTAAAGGCATTTTACAGTTATGGTGACTTCCC
ACAACGCGTAGCAACATGCAAATATCGCAGGGAGTACCGCCCCTGTCCCTTGTACGCGTCTTCTCAGGACGCACG
CACGCGCGCTGTGTTCCCGCCCTGTGACTCCAGGCGGGCATTTCCCGGGGCGGGTTTGCTGACAGGAACGTTCAG
GCTCC
>H1_196-H1_197 (SEQ ID NO: 1145)
TGAGAAAGGGTGGCTCCACAGAGCATTTATAAGGCTCCCATACCTAAAGACATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCTCGGGAGGGGGTTGCTGACGGGAACGTTCAG
GCTCC
>H1_199-H1_200 (SEQ ID NO: 1146)
TGGGGAAAAACAGCTCACGGCGGCATTTATAAGACTCACAGATCTAAAGCCATTTCACGAATAGGGTGACTTCCC
ACAATACACAGCGACATGCAAACATAGCGGGGCGTGCCTTTCCTGTACCCTGTGGGCATCTCTCCTGGACGCACG
CGCGCCGGGTGTTCCCGCGCTGTGACTCTAGGCAAGCGCTTCCTGGGAGAGAGTTGATGACGGCAGCATTCGGGC
TCC
>H1_203-H1_199 (SEQ ID NO: 1147)
TGGGGAAAAGCGGGCTCCAGGCAGCATTTATAAGACTCACATATCTAAAGACATTTCACGGTTAGGGTGACTTCC
CACAATACACAGCGACATGCAAATATCGCGGGGCGTGCCTCCCCTGTCCCTTGTGGGCATCTTCTCGCCTGGACG
CACGCGCGCCGCGTGTTCCCGCCCTGTGACTCTAGGCGGGCGATTCCTGGGAGAGGGTTGATGACGTCAACATTC
GGGCTCC
>H1_203-H1_202 (SEQ ID NO: 1148)
CGGAGCAAACAGGCCACCAGGCAGCCTTTATAAGACTCACATATCTAAAGACATTTCACAGTTAGGGTGACTTCC
CACAGTACACAGCGATATGCAAATATCGCGGAGCGTGCCTCCCCAGTCTCTGGCGGGCATCTTCTCGCCTACACG
CACGCGCGCCGCGTGTTCCCGCCCTGTGACGCTAGGCGGGCCATTCATGGGAGAGGGTTGATGACGTCAACATTC
GGACTCC
>H1_203-H1_206 (SEQ ID NO: 1149)
TGGAGAAAAGCGGGCTCCAGGCAGCATTTATAAGACTCACATATCTAAAGACATTTCACAGTTAGGGTGACTTCC
CACAATACACAGCGACATGCAAATATCGCGGAGCGTGCCTCCCCTGTCTCTTGTGGGCATCTTCTCGCCTGGACG
CACGCGCGCCGCGTGTTCCCGCCCTGTGACGCTAGGCGGGCGATTCCTGGGAGAGGGTTGATGACGTCAACATTC
GGGCTCC
>H1_203-H1_304 (SEQ ID NO: 1150)
TGGGAAAAAGAGGGGCTTCACGCAGCATTTATAAGGCTCCCATATCTAAAGACATTTCACGGTTAGGGTGACTTC
CCCCACAATACATAGCGACATGCAAATATCATGGTCCTTCCGCGGGGCGTGCCTCCCCCTGTCCCTTGGCCCGTG
GGCATCTTCTCGCCAGGAGACGCACGCGGCGCGCTGCGTGTTCCCGCCCTGTGACTTCTAGGCGGGCGATTCCCT
GGGAGAGGGTTGGATGACGTCAGCATCGCCAACATTCGGGCTCC
>H1_206-H1_207 (SEQ ID NO: 1151)
TGAAGAAAGGCGGCTCTAAGCAGCATTTATAAGACTCACATATCTGAAGACATTTCACAGTTAGGGTGACTTCCC
ACAAGACACAGCGACATGCAAATATCGCGGAATGTGCTTCCCCTGTCTCCTGTGGGCATCTTCTCGCCTGGACGC
ACGCGCACCGCGTGTTCCCGCCCTGTGACGCTAGGCGGGCGATTCCTGGGAGAGGGTTGATGACGTCAACACTCG
GGCTCC
>H1_210-H1_208 (SEQ ID NO: 1152)
TGGGAAAGGGTGGTCCCATACAGAACTTATAAGATTCCCATATCCAAAGACATTTCACGTTTATGGTGATTTCCC
AGAACACATAGCGACATGCAAATATTGCAGGGCGCCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_209 (SEQ ID NO: 1153)
TGGGAAAGGGTGGTCCCACACAGAACTTATAAGACTCCCATATCCAAAGACATTTCACGTTTATGGTGATTTCCC
AGAACACATAGCGACATGCAAATATTGCAGGGCGCCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_212 (SEQ ID NO: 1154)
TGGGGAAGGGTGGTCCCATACAGAACTTATAAGATTCCCATACCCAAAGACATTTCACGTTTATGGTGACTTCCC
AGAAGACATAGCGACATGCAAATATTGCAGGGCGTCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_220 (SEQ ID NO: 1155)
TGGGGAAGGGTGGTCCCATACAGAACTTATAAGATTCCCATACCCAAAGACATTTCACGTTTATGGTGACTTCCC
AGAAGACATAGCGACATGCAAATATTGCAGGGCGTCACTCCCCCTGTCCCTCAACAGTCATCTTCCTGCCAGGGC
GCACGCGCGCTGGGTGTTCCCGCCTAGTGACACTGGGCCCGCGATTCCTTGGAGCGGGTTGATGACGTCAGCGTT
CGAATTCC
>H1_210-H1_225 (SEQ ID NO: 1156)
TGGGAAAGGGTGGTCCCATACAGAACTTATAAGATTCCCATACCCAAAGACATTTCACGTTTATGGTGACTTCCC
AGAACACATAGCGACATGCAAATATTGCAGGGCGTCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_213-H1_219 (SEQ ID NO: 1157)
TGGGGAAAGGTGGTCCCATACAGAACTTATAAGATTCCCATACTCAAAGACATTTCACGTTTATGGTGACTTCCC
AGAAGACACAGCGACATGCAAATATTGTAGGGCGTCACACCCCCTGTCCCTCACAGTCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_219-H1_218 (SEQ ID NO: 1158)
TGGGGAAAGGTGGTCCCACACAGAACTTATAAGATTCCCATACTCAAAGACATTTCTCGTTTATGGTGACTTCCC
AGAAGACACAGCGACATGCAAATATTGTAGGGCGTCACACCCCTGTCCCTCACAGTCATCTTCCTGCCAGGGCGC
ACGCGCGCTGGGTGTTCCCGCGTAGTGACACTGGGCCCGCGATTCCTTGGAGCGGGTTGATGACGTCAGCGTTCG
AATTCC
>H1_220-H1_222 (SEQ ID NO: 1159)
TGGGGAAGGGTGGTCCCATACAGAACTTATAAGATTCCCATACTCAAAGACATTTCACGTTTATGGTGACTTCCC
AGAAGACATAGCGACATGCAAATATTGCAGGGCGTCACACCCCCTGTCCCTCAACAGTCATCTTCCTGCCAGGGC
GCACGCGCGCTGGGTGTTCCCGCGTAGTGACACTGGGCCCGCGATTCCTTGGAGCGGGTTGATGACGTCAGCGTT
CGAATTCC
>H1_220-H1_223 (SEQ ID NO: 1160)
TGGGGAAGGGTGGTCCTACACAGAACTTATAAGACTCCCATACTCAAAGACATTTCACGTTTATGGTGACTTCCC
AGAAGACATAGCGACATGCAAATATTGCAGGGCGTCACACCCCCTGTCCCTTACAGCCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_220-H1_224 (SEQ ID NO: 1161)
TGGGGAAGGGTGGTCCTACACAGAACTTATAAGACTCCCATACTCAAAGACATTTCACGTTTATGGTGACTTCCC
AGAAGACATAGCGACATGCAAATATTGCAGGGCGTCACACCCCCTGTCCCTTAACAGTCATCTTCCTGCCAGGGC
GCACGCGCGCTGGGTGTTCCCGCGTAGTGACACTGGGCCCGCGATTCCTTGGAGCGGGTTGATGACGTCAGCGTT
CGAATTCC
>H1_222-H1_213 (SEQ ID NO: 1162)
TGGGGAAGGGTGGTCCCATACAGAACTTATAAGATTCCCATACTCAAAGACATTTCACGTTTATGGTGACTTCCC
AGAAGACATAGCGACATGCAAATATTGCAGGGCGTCACACCCCCTGTCCCTCACAGTCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_227-H1_210 (SEQ ID NO: 1163)
TGGGGAAGGGTGGTCCCACACAGAACTTATAAGATTCCCATACCCAAAGACATTTCACGATTATGGTGACTTCCC
AGAAGACATAGCGACATGCAAATATTGCAGGGCGTGCCTCCCCCTGTCCCTCAACAGTCGTCTTCCTGCCAGGGC
GCACGCGCGCTGGGTGTTCCCGCCTAGTGACACTGGGCCCGCGATTCCTTGGAGCGGGTTGATGACGTCAGCGTT
CGAATTCC
>H1_227-H1_226 (SEQ ID NO: 1164)
TGGGGAAGGGTGGTCCTACACAGAACTTATAAGATTCCCATACCCAAAGACATTTCACGATTATGGTGACTTCCC
AGAAGACACAGCGACATGCAAATATTGCAGGTCGTGCCTCGCCTGTCCCTCACAGTCGTCTTCCTGCCAGGGCGC
ACGCGCGCTGGGTGTCCCGCCAACTGACACTGGGCTCGCGATTCCTTGGAGCGGGTTGATGACGTCAGCGTTCGA
ATTCC
>H1_227-H1_228 (SEQ ID NO: 1165)
TGGGGAAGGGTGGTCCCACACAGAACTTATAAGATTCCCATACCCAAAGACATTTCACGATTATGGTGACTTCCC
AGAAGACACAGCGACATGCAAATATTGCAGGTCGTGCCTCGCCTGTCCCTCACAGTCGTCTTCCTGCCAGGGCGC
ACGCGCGCTGGGTTTCCCGCCAACTGACACTGGGCTCGCGATTCCTTGGAGCGGGTTGATGACGTCAGCGTTCGA
ATTCC
>H1_227-H1_230 (SEQ ID NO: 1166)
TGGGGAAGGGTGGTCCTACGCAGAACTTATAAGATTCCCATACCCAAAGACATTTCACGATTATGGTGACTTCCC
AGAATACACAGCGACATGCAAATATTGCAGGTCGTGCCTCGCCTGTCCCTCACAGTCGTCTTCCTGCCAGGGCGC
ACGCGCGCTGGGTGTCCCGCCAACTGACACTGGGCTCGCGATTCCTTGGAGCGGGTTGATGACGTCAGCGTTCGA
ATTCC
>H1_231-H1_232 (SEQ ID NO: 1167)
TGAGGAAAAATGGTTCCACACAGAATTTATAAGGTTCCCAAATCTAAAGACATTTCACCATTATGGTGATTTCCC
ACAACACATAGCGACATGCAAATATCTCAGAGCGTACCTCCCCTGTCCTATACGGGCGTCAACTCGCCAGGGCGC
ACGCGCGCTGTGTGTTTCCCGCCTGTGACTCGGGACTCTGGGCCCGCGATTCCTCGGAGCGGGTTGAGAACGTCA
GCTCCGGTGCTTC
>H1_233-H1_231 (SEQ ID NO: 1168)
TGAGGAAAAGTGGTTCCACACAGAATTTATAAGGTTCCCAAATCTAAAGACATTTCACCATTATGGTGATTTCCC
ACAACACATAGCGACATGCAAATATCTCAGAGCGTACCTCCCCTGTCCTATACGGGCGTCAACTCGCCAGGGCGC
ACGCGCGCTGTGTGTTTCCCGCCTGTGACTCGGGACTCTGGGCCCGCGATTCCTCGGAGCGGGTTGATAACGTCA
GCTCCGGTGCTTC
>H1_234-H1_235 (SEQ ID NO: 1169)
TGGGAAAAGGTGGGCCCACACAGAATTTATAAGGCTCCCATACCTAAAGACATTTCACGATTATGGTGACTTCCC
ACAATACATAGCGACATGCAAATATCGCGGGGCGTGCCTCCCCTGTCCCGTACCCCACAGGCGTCTTCTCGCCAG
GGCGCACGCGCGCTGCGTGTTCCCGCCCTGTGACTAGGGATTCTGGGCCCGCGATTCCTGGGAGCGGGTTGATGA
CGTCAGCGTTCGGGCTCC
>H1_235-H1_233 (SEQ ID NO: 1170)
TGAGGAAAAGTGGGCCCACACAGAATTTATAAGGTTCCCAAACCTAAAGACATTTCACCATTATGGTGACTTCCC
ACAATACATAGCGACATGCAAATATCTCAGGGCGTGCCTCCCCTGTCCCGTACCCCACGGGCGTCAACTCGCCAG
GGCGCACGCGCGCTGCGTGTTTCCCGCCTGTGACTCGGGACTCTGGGCCCGCGATTCCTGGGAGCGGGTTGATGA
CGTCAGCTCTGGGGCTTC
>H1_238-H1_239 (SEQ ID NO: 1171)
TGGCAGAAAGCGGCCCGCCGCCGCATTTATAAGGCTCTCCCACCTAAAGCCATATAATGGTTATGGTGACTTCCC
AGAATACATGGCAACATGCAAATATCGTGCGGTATACCTCCCCTGTCGCGCGTAGGCGTCTCCTCCCCTGGACGC
ACGGGCGCCGCATGTTCCCGCCCTATGACTCTGGGCCGGCGACTACGGGAGAGAGCTGATGACGTGACCGCGACC
GCTCGGGCTCC
>H1_241-H1_238 (SEQ ID NO: 1172)
TGGGAAAAAGCGGCCCCCCGCCGCATTTATAAGGCTCTCCCACCTAAAGACATTTAACGGTTATGGTGACTTCCC
ACAATACATAGCAACATGCAAATATCGCGCGGTATACCTCCCCTGTCGCGCGTAGGCGTCTCCTCCCCTGGACGC
ACGGGCGCTGCGTGTTCCCGCCCTGTGACTCTGGGCCGGCGACTACGGGAGAGAGCTGATGACGTGACCGCGACC
GCTCGGGCTCC
>H1_242-H1_243 (SEQ ID NO: 1173)
TGGGAAGTAAGAGATTCACGCCGGTTATATAAGATTCCTGTAACTAAAGAAATTTCAAGGATAGGGTGACTTCCC
ACAATACAAAGCGACATGCAAATATCGCGGGGCGTGCCTGTCCTGACCTTTGTGAGACTCTTCGCTAGGACGCAG
GCGTGCTGCGAGTTCCCGCCTTATCGGCGAGTCCTGGGGGAGAGTTGATGACGCCAACATTCGGGCTCC
>H1_242-H1_248 (SEQ ID NO: 1174)
TGGGAAAAAAAGGCTTCACGCAGATTATATAAGGTTCCTGTACCTAAAGACATTTCAAGGTTAGGGTGACTTCCC
ACAATACATAGCGACATGCAAATATAGCGGGGCGTGCCTCCCCTGTCCCTTGTGGGCGTCTTCTCGCTAGGACGC
ACGCGCGCTGCGTGTTCCCGCCTTGTGACTCTAGGTCGGCGAGTCCTGGGAGAGGGTTGATGACGTCAACATTCG
GGCTCC
>H1_247-H1_246 (SEQ ID NO: 1175)
TGCGTAAAATACGCTTCTCGCAGATTATATAAGGTTCCTGTACCTAAAGACATTTCAAGGGTAGGGTGACTTCCC
ACAACACATAGCGACATGCAAATATAGGGTGTGTCTCCCCTGGCCCTTGTGGGCGTCTTCTCGCTAGGACGCACG
CGCGCTGCGTTTTCCCGCCTTCTGGCTCTAGGTCGGCGAGTCCCGGGAAAGGATTGATTACGTCAACATTCGGGC
TTC
>H1_248-H1_247 (SEQ ID NO: 1176)
TGCGTAAAAAAGGCTTCACGCAGATTATATAAGGTTCCTGTACCTAAAGACATTTCAAGGTTAGGGTGACTTCCC
ACAATACATAGCGACATGCAAATATAGGGGGGTGTGTCTCCCCTGGCCCTTGTGGGCGTCTTCTCGCTAGGACGC
ACGCGCGCTGCGTTTTCCCGCCTTGTGACTCTAGGTCGGCGAGTCCTGGGAAAGGATTGATTACGTCAACATTCG
GGCTTC
>H1_248-H1_249 (SEQ ID NO: 1177)
TGCGTAAAAAAGGCTTCACGGTGACTATATAAGGTTCCTGTACCTAATGACATTTCAAGATTAGGGTGACTTCCC
ACAATACATAGCGACATGCAAATAAAGGGGGGTTTCTCGTCTGTCCCCCCTGTGGGCGTCTTCTTGCTAGGACGC
ACGCGCGCTGCGTTTTCCCGCCTTGTGATTCTGGGTCGGCAAGTCCTGGGAAAGGATTGATTACGTCAACATTCG
GGCTTC
>H1_250-H1_251 (SEQ ID NO: 1178)
TGAGAAAAAAAGGCCACACGGAGAATATATAAGGCTCCCATATCTGAAGACATTTTAAGATTAGGGTGATTTCCC
ACAATACATAGCGACATGTAAATGTAGTGGGGCATGCCTTCCCTGTCCCTTGTGGGCAGCTTCTCGCCAGGACGC
ACGCGCGCTGCGTGTTCCCGCCTTGTGACTAAATTGGCGAGTCTGGGAGGAGACTGATGATGTCAGCATCATCAA
CTTTCCCGCTCC
>H1_251-H1_252 (SEQ ID NO: 1179)
TGAGGGAAGACTGTCGTAGGGAGAATATATAAGGCTCCCATATCGCTAGACATTTTAAGATGAGGGTGATTTCCC
ACAATGCATAGCGACATGTAAATGAAGTGGGGCATGCTTTCCCTGTCCCTTGTGGGCAGCTTCTCGCCAGGACGC
ACGCGCGCTGCGTGTTCCCGCCTTGTGACTAAATTGGCGAGTCTGGGAGGAGACTGATGATGTCAGCATCATCAA
CTTTCCCGCTCC
>H1_253-H1_242 (SEQ ID NO: 1180)
TGGGAAAAAAAGGCTTCACGCAGAATATATAAGGCTCCCATATCTAAAGACATTTCAAGGTTAGGGTGACTTCCC
ACAATACATAGCGACATGCAAATATAGCGGGGCGTGCCTCCCCTGTCCCTTGTGGGCATCTTCTCGCCAGGACGC
ACGCGCGCTGCGTGTTCCCGCCTTGTGACTCTAGGCTGGCGAGTCCCTGGGAGAGGGTTGATGACGTCAGCATCG
TCAACATTCGGGCTCC
>H1_253-H1_250 (SEQ ID NO: 1181)
TGAGAAAAAAAGGCCTCACGCAGAATATATAAGGCTCCCATATCTGAAGACATTTTAAGATTAGGGTGATTTCCC
ACAATACATAGCGACATGTAAATGTAGTGGGGCATGCCTCCCCTGTCCCTTGTGGGCAGCTTCTCGCCAGGACGC
ACGCGCGCTGCGTGTTCCCGCCTTGTGACTAAATTGGCGAGTCTGGGAGGAGATTGATGATGTCAGCATCATCAA
CTTTCCCGCTCC
>H1_253-H1_255 (SEQ ID NO: 1182)
CGCGAGAAAAATTCTTCACGCAGAATATATAAGGATCCCATATCTGAAGACATTTTACGATTACGGTGATTTCCC
ACAACACATAGCGACATGTAAATGTAGTGGGGCATGCCTCCCCTGTCCCTTGTGGGCAGCTTCTCGCCAGAACGC
ACGCGCGGTGCGTGTTCCCGCCTTGTGACTAAGTTGGCGAGTCAGGGAGGAGATTGATGATGTCATCATCGTCAG
CTCACCCGCTCC
>H1_253-H1_256 (SEQ ID NO: 1183)
CGAGAGAAAAAGTCTTCACGCAGAATATATAAGGATCCCATATCTGAAGACATTTTACGATTACGGTGATTTCCC
ACAACACATAGCGACATGTAAATGTAGTGGGGCATGCCTCCCCTGTCCCTTGTGGGCAGCTTCTCGCCAGAACGC
ACGCGCGGTGCGTGTTCCCGCCTTGTGACTAAGTTGGCGAGTCAGGGAGGAGATTGATGATGTCATCATCGTCAG
CTCACCCGCTCC
>H1_253-H1_257 (SEQ ID NO: 1184)
TGAGAAAAAAAGGCCTCACGCAGAATATATAAGGCTCCCATATCTGAAGACATTTTAAGGTTAGGGTGATTTCCC
ACAATACATAGCGACATGTAAATGTAGTGGGGCATGCCTCCCCTGTCCCTTGTGGGCAGCTTCTCGCCAGGACGC
ACGCGCGCTGCGTGTTCCCGCCTTGTGACTAAATTGGCGAGTCTGGGAGGAGATTGATGACGTCAGCATCATCAA
CTTTCCCGCTCC
>H1_253-H1_258 (SEQ ID NO: 1185)
TGAGAAAAAAAGGCCTCACGCAGAATATATAAGGCTCCCATATCTGAAGACATTTTAAGGTTAGGGTGATTTCCC
ACAATACATAGCGACATGCAAATATAGTGGGGCGTGCCTCCCCTGTCCCTTGTGGGCAGCTTCTCGCCAGGACGC
ACGCGCGCTGCGTGTTCCCGCCTTGTGACTAAATTGGCGAGTCTGGGAGGGGATTGATGACGTCAGCATCATCAA
CTTTCCCGCTCC
>H1_253-H1_261 (SEQ ID NO: 1186)
TGGGAAAAAGAGGGCTTCACGCAGAATATATAAGGCTCCCATATCTAAAGACATTTCACGGTTAGGGTGACTTCC
CCCACAATACATAGCGACATGCAAATATCATGGTCCTTCAGCGGGGCGTGCCTCCCCTGTCCCTTGTGGGCATCT
TCTCGCCAGGACACGCACGCGGCGCGCTGCGTGTTCCCGCCTTGTGACTTCTAGGCGGGCGAGTCCCTGGGAGAG
GGTTGGATGACGTCAGCATCGCCAACATTCGGGCTCC
>H1_253-H1_407 (SEQ ID NO: 1187)
TGGGAAAAAAAGGCTTCACGCAGAATATATAAGGCTCCCATATCTAAAGACATTTCAAGGTTAGGGTGACTTCCC
CCACAATACATAGCGACATGCAAATATCATGGTCCTTCAGCGGGGCGTGCCTCCCCTGTCCCTTGTGGGCATCTT
CTCGCCAGGACGCACGCGCGCTGCGTGTTCCCGCCTTGTGACTCTAGGCTGGCGAGTCCCTGGGAGAGGGTTGAT
GACGTCAGCATCGTCAACATTCGGGCTCC
>H1_261-H1_259 (SEQ ID NO: 1188)
CGGGAAAAAAACGGCTTCTGGTGGAAAATATATGAGGCCCATACCTGAAGACCTTTCACGGTTATGGTGACTTCC
CACAATACATAGCGACATGCAAATATAGTGGGGCGTGCCTCCACTGTCCTTTGCGGGCATCGTCTCGCCAGGAAG
CGCGCGCTGCGTGTTCCCGCCTTTTGACTCTAGGCGGGCGAATCCTGGGAGAGGGTTGTTGACGTCAACATTCGG
GCTCC
>H1_261-H1_260 (SEQ ID NO: 1189)
CAAGAGAAAACCGAGCCCTGCTGGAAAATATATGAGGCCCACTCTTCAAGACCTTTTATGGTTATGGTAACTTCC
CATAACACATAGCGACATGCAAATATCGTGGGGTGTGCCTCCACGGTCCTTTGCGGACACCGTCTTGCCCGTAAG
CGCGCTGGGTATTCCCGCCTTCTGACTCTAGGCGGGCGAATCCTAGGAGAGGGTTGTTGACGTCGACATTCGGGC
ACC
>H1_261-H1_264 (SEQ ID NO: 1190)
CAAGAGAGAAACGTGCCCTGCTGGAAAATATATGAGGCCCATTCCTCAAGACCTTTTATGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_265 (SEQ ID NO: 1191)
CAAGAAAGAAACGTCCTCTGGTGGAAAATATATGAGGCCCATTCCTCAAGACCTTTTACGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_268 (SEQ ID NO: 1192)
CAAGAAAGAAACGTCCTCTGGTGGAAAATATATGAGGCCCATTCCTCAAGACCTTTTACGGTTATGGTGACTTCC
CACAATACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_269 (SEQ ID NO: 1193)
CAAGAAAGAAACGTGCTCTGGTGGAAAATATATGAGGCCCATTCCTCAAGACCTTTTACGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_270 (SEQ ID NO: 1194)
CGGGAAAAAAACGGCCTCTGGTGGAAAATATATGAGGCCCATACCTGAAGACCTTTCACGGTTATGGTGACTTCC
CACAATACATAGCGACATGCAAATATCGTGGGGCGTGCCTCCACTGTCCTTTGCGGGCATCGTCTCGCCCGGAAG
CGCGCGCTGTGTGTTCCCGCCTTTTGACTCTAGGCGGGCGAATCCTGGGAGAGGGTTGTTGACGTCAACATTCGG
GCTCC
>H1_261-H1_272 (SEQ ID NO: 1195)
TGGGAAAAAGAGGGCTTCACGCGGAATATATAAGGCTCCCATACCTAAAGACCTTTCACGGTTAGGGTGACTTCC
CCACAATACATAGCGACATGCAAATATAGTGGGGCGTGCCTCCCCTGTCCCTTGCGGGCATCTTCTCGCCAGGAC
ACGCGCGCGCCGCGCTGCGTGTTCCCGCCTTTTGACTTCTAGGCGGGCGAATCCTGGGAGAGGGTTGGATGACGT
CCAACATTCGGGCTCC
>H1_261-H1_292 (SEQ ID NO: 1196)
CGGGAAAAAAAGGGCTTCTGGCGGAAAATATATGAGGCCCATACCTGAAGACCTTTCACGGTTATGGTGACTTCC
CACAATACATAGCGACATGCAAATATAGTGGGGCGTGCCTCCCCTGTCCCTTGCGGGCATCTTCTCGCCAGGAAG
CGCGCGCGCTGCGTGTTCCCGCCTTTTGACTCTAGGCGGGCGAATCCTGGGAGAGGGTTGATGACGTCAACATTC
GGGCTCC
>H1_263-H1_271 (SEQ ID NO: 1197)
CAAGAGAGAAACTTGTCGTGCTGGAAAATATATGAGGCCCATTCCTCAGGACCTTTTATGGTTAGGGTGATTTCC
CACAATACATAGCGACATGCAAATATAGTGGGGTGTGCTTCCACTGTCCTTTGCGGACACCGTCTCGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_264-H1_263 (SEQ ID NO: 1198)
CAAGAGAGAAACTTGTCGTGCTGGAAAATATATGAGGCCCATTCCTCAGGACCTTTTATGGTTAGGGTGACTTCC
CACAACACATAGCGACATGCAAATATCGTGGGGTGTGCTTCCACTGTCCTTTGCGGACACCGTCTCGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_266-H1_267 (SEQ ID NO: 1199)
CGAGGAAATAATCTCCCCTGGTGGCAAATATAGGAAGCCCATTCCTCAAGACCTTTTAAGGTTACGGTGACTTCC
CACAATACATAGCAACATGCAAATATTGTGGGGTGTGCCTTCACTGTCCTTTGCGGTCACTGTCTTGCCCATAAG
CGCGCTGTGTAATCCCGCCTTTTGACGTTAGGCAGGCGAATCCTGGGAGAGGGTTGCTGACGTCGACATTCGGCT
CC
>H1_268-H1_266 (SEQ ID NO: 1200)
CAAGGAAGTAACGTCCTCTGGTGGAAAATATATGAGGCCCATTCCTCAAGACCTTTTACGGTTATGGTGACTTCC
CACAATACATAGCAACATGCAAATATCGTGGGGTGTGCCTCCACTGTCCTTTGCGGACACTGTCTTGCCCGTAAG
CGCGCTGTGTAATCCCGCCTTTTGACTCTAGGCGGGCGAATCCTGGGAGAGGGTTGTTGACGTCGACATTCGGCT
CC
>H1_272-H1_273 (SEQ ID NO: 1201)
GGGGAGAAGGCGCTTTCCGCGGATTATATAAGGCTCCAGCACCTAGAGGCCTTTAACAGTTAGGGTGATTTCCCA
CAATGCATAGCGACATGCAAATATAGTTGGGTGTGCTTTCCCTGTTCCTTGCCTGCATCTTCTTGCCTGCGTGTT
CCCGCCTTTTGACTGCAGGCGGGCGAATCCTGGGAGAGAGTTGATGACGTCAACACTCAGGCTCC
>H1_272-H1_274 (SEQ ID NO: 1201)
GGGGAGAAAGGGGCTTCACGCGGAATATATAAGGCTCCCGTACCTAAAGGCCTTTCACGGTTAGGGTGACTTCCC
CACAATACATAGCGACATGCAAATATAGTTGGGCGTGCCTCCCCTGTCCCTTGCGGGCATCTTCTCGCCAGGACA
CGCGCGCGCCGCGCTGCGTGTTCCCGCCTTTTGACTTCCAGGCGGGCGAATCCTGGGAGAGGGTTGGATGACGTC
CAACATTCGGGCTCC
>H1_274-H1_291 (SEQ ID NO: 1202)
GGGGAGAAAGGGGCTTCACGGCGAATATATAAGGCTCCCGTACCTAAAGGCCTTTCACGGTTAGGGTGACTTCCC
CACAATACATAGCGACATGCAAATATAGTTGGGCGTGCCTCCCCTGTCCCTTGCGGGCATCTTCTCGCCCGGACA
CGCGCGCGCCGCGCTGCGTGTTCCCGCCTTTTGACTTCCAGGCGGGCGAATCCTGGGAGAGGGTTGGATGACGTC
CAACATTCGGGCTCC
>H1_276-H1_280 (SEQ ID NO: 1203)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTGGATGGGCGTGCCTCCCCGGTCCCTGCCGGCAACTTCTCTCCGGGACGCG
CGCTCGCGCTGAGTGTTCCCGCCTTTTGACGCCAGCGGAGCGAATCCGGGGAGCGGGCGGATGACGTCAACAGTG
CGGCTCC
>H1_279-H1_276 (SEQ ID NO: 1204)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTAGATGGGCGTGCCTCCCCGGTCCCTGCCGGCAACTTCTCTCCGGGACGCG
CGCTCGCGCTGAGTGTTCCCGCCTTTTGACGCCAGCCGAGCGAATCCGGGGAGCGGGCGGATGACGTCAACAGTG
CGGCTCC
>H1_280-H1_277 (SEQ ID NO: 1205)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTGGATGGGCGTGCCTCCCCGGTCCCTGCCAGCAACTTCTCTCCGGGACGCG
CGCTCGCGCTGAGTGTTCCCGCCTTTTGACGCCAGCGGAGCGAATCCGGGGAGCGGGCGGATGACGTGAACAGTG
CGGCTCC
>H1_282-H1_279 (SEQ ID NO: 1206)
GGGAAGAGAGCCTCACACGGCGGCTATATAAGGCCCCCTTACCTATAGGCCTTTTACGGTTAGGGTGACTTCCCC
ACAACACATAGCGACATGCAAATTTAGATGGGCGTGCCTCCCCTGTCCCTGTGGGCAACTTCTCTCCGGGACACG
CGCGCTCGCGCTGAGTGTTCCCGCCTTTTGACTTCCAGCCGAGCGAATCCTGGGAGAGGGCAGATGACGTCAACA
GTCAGGCTCC
>H1_282-H1_281 (SEQ ID NO: 1207)
GGGAAGAGGGCCTCACACGAGGGCTATATAAGGCCCCCTTACCTATAGGCCTTTTACGGTTAGAGTGACTTCCCA
CAACACCTAGCGACATGCAAATTTAGATGGGCGTGCCTCCTCTGTCCCTGTGGCAACACCTCTCCGGGACGCGCG
CTCGCTCTGAGTGTTCCCGCCTTTTGACTTCCAGCCGAACGAATCCTGGGAGAGGGCAGATGACGTCAATAGTCA
GGCTCC
>H1_282-H1_283 (SEQ ID NO: 1208)
GGGAAGAGGGCCTCACACGAGGGCTATATAAGGCCCCCTTACCTATAGGCCTTTTATGGTTAGAGTGACTTCCCA
CAACACCTAGCGACATGCAAATTTAGATGGGCGTGCCTCCTCTGTCCCTGTGGCAACACCTCTCCGGGACGCGCG
CTCGCTCTGAGCGTTCCCGCCTTTTGACTTCCAGCCGAACGAATCCTGGGAGAGGGCAGTGACGTCAATAGTCAG
GCTCC
>H1_282-H1_284 (SEQ ID NO: 1209)
GGGAAGAGAGCCTCACACGGCGGCTATATAAGGCCCCCTTACCTATAGGCCTTTTACGGTTAGGGTGACTTCCCA
CAACACATAGCGACATGCAAATTTAGATGGGCGTGCCTCCCCTGTCCCTGTGGGCAACTTCTCTCCGGGACACGC
GCGCTCGCGCTGAGTGTTCCCGCCTTTTGACTTCCAGCCGAGCGAATCCTGGGAGAGGGCAGATGACGTCAACAG
TCAGGCTCC
>H1_285-H1_282 (SEQ ID NO: 1210)
GGGAAGAGAGGCCTACACGGCGGCTATATAAGGCCCCCTTACCTATAGGCCTTTTACGGTTAGGGTGACTTCCCC
ACAACACATAGCGACATGCAAATTTAGATGGGCGTGCCTCCCCTGTCCCTGTGGGCAACTTCTCTCCGGGACACG
CGCGCTCGCGCTGAGTGTTCCCGCCTTTTGACTTCCAGCCGAGCGAATCCTGGGAGAGGGCAGATGACGTCAACA
GTCAGGCTCC
>H1_287-H1_285 (SEQ ID NO: 1211)
GGGAAGAGAGGCACTACACGGCGGCTATATAAGGCCCCCTTACCTATAGGCCTTTTACGGTTAGGGTGACTTCCC
CACAACACATAGCGACATGCAAATTTAGATGGGCGTGCCTCCCCTGTCCCTGTGGGCAACTTCTCTCCGGGACAC
GCGCGCTCCGCGCTGAGTGTTCCCGCCTTTTGACTTCCAGCCGAGCGAATCCTGGGAGAGGGCAGATGACGTCCA
ACAGTCAGGCTCC
>H1_287-H1_288 (SEQ ID NO: 1212)
GGGAGAAGGGGGAGTACACGGCGGATATATAAGGCCCCCTTATGTATAGTCCTTTTACGGTTAGGGTGACTTCCC
ACAACGCATAGCGACATGCAAATTTGACGGGCGTGCCTCCTCTGTCCCTGCGGGCAACTTCTCTCCTGGACGCGC
GCTCCGCGCTGCGTGTTCCCGCCTTTTGACTCCAGCCGAGCGAATCCTGGGAGAGGGCAGGTGACGTCCAACAGT
CAGGCTCG
>H1_287-H1_290 (SEQ ID NO: 1213)
GAGAGAGGCTGTGCACACGGCGGATATATAAGGCCCCCTTATGTATAATCCTTTACCGGTTAGGGTGACTTCCCA
CAACGCATAGCGACATGCAAATTTGACGGGCGTGCCTCCTCTGTCCCTGCGGGCAACTTCTCTCCTGGACGCGCG
CTCCGCGCTGCGTGTTCCCGCCTTTTGACTCCAGCCGAGCGAATCCTGGGAGAGGGCAGGTGACGTCCAACAGTC
AGGCTCG
>H1_288-H1_289 (SEQ ID NO: 1214)
GGGAGAAGGGGGAGTACACGGCGGATATATAAGGCCCCCTTATGTATAGTCCTTTTACGGTTAGGGTGACTTCCC
ACAACGCATAGCGACATGCAAATTTGACGGGCGTGCCTCCTCTGTCCCTGCGGGCAACTTCTCTCCTGGACGCGC
GCTCGCGCTGCGTGTTCCCGCCTTTTGACTCCAGCCGAGCGAATCCTGGGAGAGGGCAGGTGACGTCAACAGTCA
GGCTCG
>H1_291-H1_287 (SEQ ID NO: 1215)
GGGAAGAGAGGCACTACACGGCGGCTATATAAGGCCCCCTTACCTATAGGCCTTTTACGGTTAGGGTGACTTCCC
CACAACACATAGCGACATGCAAATTTAGATGGGCGTGCCTCCCCTGTCCCTTGTGGGCAACTTCTCTCCGGGACA
CGCGCGCTCCGCGCTGAGTGTTCCCGCCTTTTGACTTCCAGCCGAGCGAATCCTGGGAGAGGGCAGGATGACGTC
CAACAGTCAGGCTCC
>H1_294-H1_295 (SEQ ID NO: 1216)
TAGAAAAAATCGTAGTTTATGCTGGATTTATAAGATTCCCACATCTAAAGCCATTTCACAGTTACGGTGAACTTC
CCACTACACACGGCGATATGCAAATATAGCGGAAGTGTTCCTGAGGCGTGGTAAAGCGCGCGCGCGCTGAGAGTT
CCCGCCCTGTGGTGCTGGGCTGGAGATGCCTGAGAACTGGCTGATGACGGCAACGTTCGGGCTCC
>H1_295-H1_296 (SEQ ID NO: 1217)
TAGAAAAAATCGTGCCTATGCTGGATTTATAAGATTCCCACATCTAAAGCCATTTCTCAGTTACGGTGAACTTCC
CACTACACACGGCGATATGCAAATATAGCGGAAGTGTTCCTGAGGCGTGGTAAAGCGCGCGCGCGCTGAGAGTTC
CCGCCCTGTGGTGCTGGGCTGGAGATGCCTGAGAACTGGCTGATGACGGCAACGTTCGGGCTCC
>H1_296-H1_297 (SEQ ID NO: 1218)
TAGAAAAAATCGTGCCTACGCTGGATTTATAAGATTCCCACATCTAAAGCCATTTCTCAGTTACGGTGAACTTCC
CACTACACACGGCGATATGCAAATATAGCGGAAGTGTTCCTGAGGCGTGGTAAAGCGCGCGCGCGCTGAGAGTTC
CCGCCCTGTGGTGCTGGGCTGGAGATGCCTGAGAACTGGCTGATGACGGCAACGTTCGGGCTCC
>H1_298-H1_294 (SEQ ID NO: 1219)
TAGAAAAAATGGTAGTTTATGCGGGATTTATAAGACTCCCACATCTAAAGCCATTTCACAGTTACGGTGACTTCC
CCACAACACACGGCGATATGCAAATATAGCGGAAGTGTTCCTGAGGCGTGGTAAAGCGCACGCGCGCTGAGAGTT
CCCGCCCTGTGGTGCTGGGCCCGAGATGCCTGAGAGCGGGCTGATGACGGCAGCGTTTGGGCTCC
>H1_299-H1_298 (SEQ ID NO: 1220)
TAGAAAAAAGGGGAGTTTATGCGGGATTTATAAGACTCCCATATCTAAAGACATTTCACAGTTATGGTGACTTCC
CCACAACACATGGCGATATGCAAATATCGCGGAGCTGGCCCTGAGGCGTGGTAAGGCGCACGCGCGCTGAGAGTT
CCCGCCCTGTGGCGCTGGGCCCGAGATTCCTGAGAGCGGGTTGATGACGGCAGCGTTTGGGCTCC
>H1_299-H1_300 (SEQ ID NO: 1221)
TAGAGAAAAGGGGGTGTTTGCGGGATTTATAAGATTCCCATTGCTAAAGACATTTCACAGTTATGGTGACTTCCC
ACAACACTTGGCGATATGCAAATATCACGGAGTTGGCCCTGAGGCGCGGCGAGACGCACGCGCGCTGAGAGTTCC
CGCCTTCTCACCCTGGGTCCAAGGTTCCTGAAGGCGGGTTGAAGACTGCAGTGTTTGGGCGCC
>H1_301-H1_299 (SEQ ID NO: 1222)
TAGGAAAAAGGGGGGTTTATGCAGGATTTATAAGACTCCCATATCTAAAGACATTTCACGGTTATGGTGACTTCC
CCACAACACATAGCGATATGCAAATATCGCGGAGCGGGCCCTGAGGCGTGGTCAGGCGCACGCGCGCTGCGAGTT
CCCGCCCTGTGGCGCTGGGCCCGAGATTCCTGAGAGCGGGTTGATGACGTCAGCGTTTGGGCTCC
>H1_301-H1_302 (SEQ ID NO: 1223)
TAGGAAACGCGCATTTTAGGCAGGATTTATAAGACACCCATATCTAAAGACATTTCACGGTTATGGTGACTTCCC
ACAACACATAGCGAAATGCAAATATGTGGAGCAGGCGCTGAGGCGTGGTCGGGCGCACGCGCGCTGCGAGTTCCC
GCCCTTCGGCGCTAGGCCCGAGATGCCTGAGAGCTGGTTGATCACGTCTGCGTTTGGACTCA
>H1_301-H1_303 (SEQ ID NO: 1224)
TAGGAAAAGAGCATTTTAGGCAGGATTTATAAGACACCCATATCTAAAGACATTTCACGGTTATGGTGACTTCCC
ACAACACATAGCGAAATGCAAATATGTGGAGCGGGCGCTGAGGCGTGGTCGGGCGCACGCGCGCTGCGAGTTCCC
GCCCTTCGGCGCTAGGCCCGAGATTCCTGAGAGCTGGTTGATGACGTCAGCGTTTGGACTCC
>H1_304-H1_253 (SEQ ID NO: 1225)
TGGGAAAAAGAGGGGCTTCACGCAGCATTTATAAGGCTCCCATATCTAAAGACATTTCACGGTTAGGGTGACTTC
CCCCACAATACATAGCGACATGCAAATATCATGGTCCTTCAGCGGGGCGTGCCTCCCCCTGTCCCTTGGCCCGTG
GGCATCTTCTCGCCAGGACACGCACGCGGCGCGCTGCGTGTTCCCGCCTTGTGACTTCTAGGCGGGCGAGTCCCT
GGGAGAGGGTTGGATGACGTCAGCATCGCCAACATTCGGGCTCC
>H1_304-H1_293 (SEQ ID NO: 1226)
CGGGAAAAAGACGGGCCTCACGCCGCATTTATAAGGCTCCCATATCTAACGACATTTTACGGTTAGGGTGACTTC
CCACAATACATAGCGATATGCAAATATAGCGGGGCGTGTCTCCCCCTGGCCCTTGGCTCGTGGGCATCGTCTCGC
CAGGACGCATGCGCGCTGCTTGTTCCCGCCTTGACTACTTGCTAGTCCTGGGAGAGGGTTGATGACGTCAACGTT
CAGACTCC
>H1_304-H1_311 (SEQ ID NO: 1227)
CCGGCATAAGACGGGCCTCACGGCGCACTTATAAGGATCCCATATCTAACGACATTTTACGGTTAGGGTGACTTC
CCACAATACATAGCGATATGCAAATATAGCGGGGCGTGTCTACTCCTGGCCCTTGGTTTGTGGGCGTCGTCTCGC
CAGGACGCATGCGCACTGCTTGTTCCCGCCTTGACTACTTGCTAGTCCTGGGAGAGGGTTGATGACGTCAACGTT
CAGACTCC
>H1_306-H1_307 (SEQ ID NO: 1228)
TCAGCGTAAAGGAGTGCGTACAAAGAATTTATAAGGCTCGCATAGCTCTAGCTGCTTCACAGTTAGGGTGACTTC
CCACAAGCCATAGCGCATGTAAATATAAGGGCGTTTGTTCCCCCGCCCCCGTCCAGGCTGCAGCATCTCTCCAGG
ACGCAGGCGCACTGAGCCTTCCCGCCCGGTCACTCCAGACCCGCCATTCCCGGGCCAGGTTAATGACGTCACACT
TAAGCTCC
>H1_306-H1_310 (SEQ ID NO: 1229)
TCAGCGTAAAGGGATGCTTACGTAGAATTTATAAGGCTCCCATACCTAAAGCCATTTCACGGTTAGGGTGACTTC
CCACAAGACATAGCGACATGCAAATATAGAGGGGCGTGCTTCCCCTGTCCCGTCCCGTAGGCGTCTTCTCGCCAG
GGACGCACGCGCGCTGCGCCCTGTTCCCGCCCTGTCACTAGGGATTCTGGGCCGGCCATTCCCCGGGCGCAGGTT
GATGACGTCACGTTTGGGCTCC
>H1_308-H1_309 (SEQ ID NO: 1230)
TCAGCGTAAAAGAATGCTTAGCTAGAATTTATAAGGCTCCCAGACCTAAAGCCATATCTCGGTTAGGGTGACTTC
CCACAAGACATAGCGACATGCAAATATAGAGGGGCGGGCTTCCCCTGTGCCTTGTAGGCGTCTTCTCACGAAGTC
GCAAGCGCGTTGCGCCCTGTTCCCGCCCTGTCACTATTGATTATTGGCCGACCTTTCCTCGGGCGGAGTCTGATG
ACGTCATCGGTTCC
>H1_310-H1_308 (SEQ ID NO: 1231)
TCAGCGTAAAGGAATGCTTACCTAGAATTTATAAGGCTCCCAGACCTAAAGCCATATCACGGTTAGGGTGACTTC
CCACAAGACATAGCGACATGCAAATATAGAGGGGCGGGCTTCCCCTGTGCCTTGTAGGCGTCTTCTCACGAAGGA
CGCACGCGCGCTGCGCCCTGTTCCCGCCCTGTCACTATTGATTATTGGCCGACCATTCCCCGGGCGCAGTCTGAT
GACGTCATTCGGTTCC
>H1_312-H1_313 (SEQ ID NO: 1232)
TGGGGGAAGCTGGGCTCGATCAGCCTTTATAAAGCTCCAAAAACTCAAGACATTTTTCCGTTACGGTGGCTTCCC
ACAGTACACAGCGACATGCAAATAGCTTGCCAATGAATTCGCGGACCGCTTCCCGCCCCGGCGCAGGCGCGCGGA
CGCTGTCTCCCCTGGACGCGCGCTCGCGGTTCCCGGGAGCTGGCTGATGACGTTCGGTCTCC
>H1_312-H1_314 (SEQ ID NO: 1233)
TGGGGAAAGGTGGGCTCAAGCAGACTTTATAAAGCTCCAAAAACTCAAGACATTTTTCCGTTACGGTGGCTTCCC
ACAATACACAGCGACATGCAAATATAGTGGAGTGTGCTTGCCAATGATTTCCCGGGCCGCTTCTCGCCACGGCGC
AGGCGCGCTGTGTGTTCCCGCCCTGGACGGGCGCGCCCGCGGTTCCCGGGAGCGGGTTGATGACGTTCGGTCTCC
>H1_314-H1_315 (SEQ ID NO: 1234)
TGGGGAGTGGTGGATCCAAGCAGACTTTATAAAGCTCCGAAGGTCCAAGGCATCTTTCCCTTACGGTGGCTTCCC
ACAAGACATAGCGATATGCAAATTTATCGATACGTGCTTCAGACGCGCTTCTCGCCGCAGCGCAAGCGCGCTGTG
TGCTGACGCGGGGGACGGGCCAGTGCGCGATTCCCGGGAGCGGGTTGATGACGTTCGATCTCC
>H1_317-H1_316 (SEQ ID NO: 1235)
TGGGGAGAGGTGGATCCGAACAGACTTTATAAAGCTCCGAAAGCCCAAGGCATCTTTCCCTTACGGTAGCTTCCC
ACAAGACATAGCGACATGCAAATTTCTTGAAGTATGCTTCAGACGCGCTTCTCGCCACAGCGCAAGCGCGCTGTG
TGCTGACGCGGGAACGGGCCAGTGCGCGGTTCCCGGGAGCGGGTTGATGACGTTAGATCTCC
>H1_318-H1_317 (SEQ ID NO: 1236)
TGGGGAGAGGTGGATCCAAACAGACTTTATAAAGCTCCGAAAGCCCAAGGCATCTTTCCCTTACGGTGGCTTCCC
ACAAGACATAGCGACATGCAAATTTATTGAAGTATGCTTCAGACGCGCTTCTCGCCGCAGCGCAAGCGCGCTGTG
TGCTGACGCGGGAGACGGGCCAGTGCGCGGTTCCCGGGAGCGGGTTGATGACGTTCGATCTCC
>H1_322-H1_319 (SEQ ID NO: 1237)
TTCAGGGTGTAGACCGGCCGCCACTATAAGGCTCGAAAGAGGAATAAATTTTTCGTTTAGGGTGATTTCCCACAA
AGCACAGCGCGTAATTTGCATGTGCTCTACCCCAGGCTCCTGTGCTAGACAAGAAGCCCGCGCATCCGGGCAAGG
GATGATGACGTCGTCCTTCAAGAGCG
>H1_322-H1_321 (SEQ ID NO: 1238)
TTCAGGGTGTAGACCGGCCGCCACTATAAGGCTCGAAAGAGGAATAAATTTTTCGTTTAGGGTGATTTCCCACAA
AGCACAGCGCGTAATTTGCATGTGCTCTATCCCAGGCTCCTGTGCCAGACAAGAAGCCCGCGCATCCGGGCAAGG
GATGATGACGTCGTCCTTCAAGAGCG
>H1_322-H1_323 (SEQ ID NO: 1239)
TTCAGTGTGTAGACCGGCCGCCACTATAAGGTTCGAAAGAGGAATAAATTTTTCGTTTAGGGTGATTTCCCACAA
AGCACAGCGCGTAATTTGCATGTGCTCTACCCCAGGCTCCTGTGCTAGACAAGAAGCCCGCGCATCCGGGCAAGG
GATGATGACGTCGTCCTTCAAGAGCG
>H1_325-H1_327 (SEQ ID NO: 1240)
TGGAGGGTGTAGACCGGCCGCCACTATAAGGCTCGAAAGAGGAATAAATTTTTCGCTTACGGTGACTTCCCACAA
AGCACAGCGCGTAATTTGCATGTGCTCTATCCCAGGCTTCCTGTTCCAGACAAGAAGCCCGCGCATCCGGGCAAG
GGATGATGACGTCATCCCCGTCCTTCAAGCGCG
>H1_328-H1_329 (SEQ ID NO: 1241)
TGGAAGGTGGAGACCTGCCGCCATAATAAGACTCCAAAAGAGAGTGAATTTAACACTTACGGTGACTTCCCACAA
AGCACAGCGTGTAATTTGCATGCGCTCTAGCCCAGGCTCCAGCTCCGGACGAGAAGCCCGCGCATCCCGGCAAAG
GATGATGACGTCGTCCTTCAAGCGCT
>H1_328-H1_332 (SEQ ID NO: 1242)
TGGAGGGTGGAGACCGGCCACCATTATAAGACTCCAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCTTCAAGCGCG
>H1_330-H1_328 (SEQ ID NO: 1243)
TGGAGGGTGGAGACCGGCCACCATTATAAGACTCCAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCCTCAAGCGCG
>H1_332-H1_325 (SEQ ID NO: 1244)
TGGAGGGTGGAGACCGGCCACCATTATAAGACTCGAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCTTCAAGCGCG
>H1_332-H1_333 (SEQ ID NO: 1245)
TACAGGGTGGAGATCGGCGAAAATTATAAGACTCGAAAGCGGCATAAAGTTTAAGCTTATGGTGACTTCCCACAA
AGCACAGCGCGTAATTTGCATGTGCTTTATCCCAGGCTCTTTCTCCAGACCAGTAGCCTGCACATCCGGGCAAGG
GGTGATGACGTCGTCCATCAAGCGCG
>H1_334-H1_330 (SEQ ID NO: 1246)
GGGAAGGTGGAGACCGGCCACCATTATAAGACTCCAAAGCGGAATACATTTTTCGGTTATGGTGACTTCCCACAA
AGCACAGCGCGTAATTTGCATGCGCTCTATCCCAGGCTTCCTGCTCCAGACAAGAAGCCCGCGCATCCGGGCAAG
GGATGATGACGTCATCCCCGTCCCTCAAGCGCG
>H1_335-H1_337 (SEQ ID NO: 1247)
ACGGCGGTGTGGAGGGCGAACTTTATAAGCCTCCGAAGAGAAAGCGATTTTTCAGTTATGGTGGTTTCCCACAAG
GCACAGCGCACAGTTTATTTGCATGCGCTCTAGCCCCGGCTCCCGCTCCAGACTAAGAAGCCCGCGCATTTCGGC
TGCGGATGATGACGTCGGGCCTCAAGCGCC
>H1_336-H1_335 (SEQ ID NO: 1248)
ACGGCGGTGTGGAGGGCGAACTTTATAAGCCTCCGAAGAGAAAGCGATTTTTCAGTTATGGTGGTTTCCCACAAG
GCACAGCGCACAGTTTATTTGCATGCGCTCCCGCCGCTTCTAGCCCCGGCTCCCGCTCCAGACTAAGAAGCCCGC
GCATTTCGGCTGCGGATGATGACGTCGGGCCTCAAGCGCC
>H1_338-H1_334 (SEQ ID NO: 1249)
GGGGAGGTGTGGGCCGGCCAGCTTTATAAGACTCCAAAGCGGAATGCATTTTTCAGTTATGGTGGCTTCCCACAA
GGCACAGCGCGCTGCTTATTTGCATGGGCTCACGCCGCTTCTAGCCCGGGCTTCCTGCTCCAGACTAAGAAGCCC
GCGCATCCCGGCCGGGCGAGGGATGATGACGTCATCCCCAGCCCTCAAGCGCG
>H1_338-H1_340 (SEQ ID NO: 1250)
GGAGGGCGGTGGCCGGCGAGCTTAATAAGCCTCGGAGGCGGGACGCCTGTTACAGTGACGGTGGTTTCCCACAAA
GCACGGCGCGGCGGTCTTGATTTGCATGCGCCTTTATGCCCGCCTCCCGCTCCGGAGAAGAAGCCCGCGCATCCC
GGCTGGGCTGGGGGTGATGACGTCAGGGCTCGAGCGCC
>H1_338-H1_342 (SEQ ID NO: 1251)
GGAGAGCGGTGGCCGGCGAGCTTAATAAGCCTCGGAAGCGGAACGCATTTTACAGTGATGGTGGTTTCCCACAAG
GCACAGCGCGGCGGCCTTTATTTGCATGCGCTTCTATTCCCGCCTCCCGCTCCAGAGAAGAAGCCCGCGCATCCC
GGCTCGGCTGGGGATGATGACGTCAGGGCTCGAGCGCC
>H1_338-H1_343 (SEQ ID NO: 1252)
GGGGTGGTGTGGCTGGCGAGCTTAATAAGGCTCCGAAGCGGAATGCATTTTACAGTGATGGTGGTTTCCCACAAG
GCACAGCGCGGCGTTTATTTGCATGCGCTTCTATTCCCGCCTCCCGCTCCAGACAAGAAGCCCGCGCATCCCGGC
TCGGCTGGGGATGATGACGTCAGGGCTCGAGCGCC
>H1_338-H1_344 (SEQ ID NO: 1253)
GGAGAGGGGTGGCCGGCGAGCTTAATAAGCCTCCGAAGCGGAACGCATTTTACAGTGATGGTGGTTTCCCACAAG
GCACAGCGCGGCGTTTATTTGCATGCGCTTCTATTCCCGCCTCCCGCTCCAGAGAAGAAGCCCGCGCATCCCGGC
TCGGCTGGGGATGATGACGTCAGGGCTCGAGCGCC
>H1_338-H1_345 (SEQ ID NO: 1254)
GGGGTGGTGTGGGTGGCGAGCTTTATAAGGCTCCGAAGCGGAATGCATTTTTCAGTTATGGTGGTTTCCCACAAG
GCACAGCGCGCCGTTTATTTGCATGGGCTCCCGCCGCTTCTAGCCCCGGCTCCCGCTCCAGACTAAGAAGCCCGC
GCATCCCGGCCCGGCTGGGGATGATGACGTCAGGCCTCAAGCGCC
>H1_338-H1_351 (SEQ ID NO: 1255)
GGGGAGGTGTGGGCGGCGAGCTTTATAAGACTCCAAAGCGGAATGCATTTTTCAGTTATGGTGGTTTCCCACAAG
GCACAGCGCGCTGCTTATTTGCATGGGCTCACGCCGCTTCTAGCCCGGGCTCCCGCTCCAGACTAAGAAGCCCGC
GCATCCCGGCCGGGCAGGGGATGATGACGTCAGCCCTCAAGCGCG
>H1_340-H1_341 (SEQ ID NO: 1256)
GCAAAGCGGTGGCCGGCGAGCTTAATAAGCCTCGGAGGCGGGACGCCTGTTACAGTGACGGTGGTTTCCCACAAA
GCACGGCGCGGCGGTCTTGATTTGCATGCGCCTTTATGCCCGCCTCCCGCTCCGGAGAAGAAGCCCGCGCATCCC
GGCTGGGCTGGGGGTGATGACGTCAGGGCTCGAGCGCC
>H1_346-H1_338 (SEQ ID NO: 1257)
GGGGAGGTGTGGGCCGGCCAGCTTTATAAGACTCCAAAGCGGAATGCATTTTTCAGTTATGGTGGCTTCCCACAA
GGCACAGCGCGCTGCTTATTTGCATGGGCTCACGCCGCTTCTAGCCCGGGCTTCCTGCTCCAGACTAAAGAAGCC
CGCGCATCCCGGCCGGGCGAGGGATGATGACGTCATCCCCAGCCCTCAAGCGCG
>H1_346-H1_347 (SEQ ID NO: 1258)
GGCGAGGGGTGGGCAGCCACCTTTATAAGACTCCAGAGCCGAATGCATTTCTCAGTTGTGGTGGCTTCCCATGAG
GCACAGCGCGCTATTTGCATGCGCTCTAGCCCGGGCTCCGGCTCTGGAATAAAAAATCCCGCGCATCCGGGTGAG
GGATGACGACGTCACCCTCAAGCGCT
>H1_349-H1_346 (SEQ ID NO: 1259)
GGGGAAGTGGGGGCAGGCCGGCTTTATAAGACTCCAGAGCGGAACGCATTTTTCAGTTATGGTGGCTTCCCACAA
GGCACAGCGCTATGCTTATTTGCATGGGCTCACGCCGCTTCTAGCCCGGGCCCCCTGCTCCAGACAAAAAAGCCC
GCGCATCCCGGCCGGGCGCGGGATGATGACGTCATCCCCAGCCCTCGAGCGCG
>H1_349-H1_348 (SEQ ID NO: 1260)
GAAGAAGTGGGGGAGACCGGCTTTATAAGACTCAGAAGGGAACAAACTTTTCAGTTGCGGTGGCTTCCCACAAGG
CACAGCGCTTTATTTGCATGCGCGCTAACCGGGGCCCCCTACTAAAAAGCCCGCGCATGCCCGGCGCGGGATGAT
GACGTCAGCCCTCGAGCGCG
>H1_349-H1_350 (SEQ ID NO: 1261)
GAAGTCGTGGGGGAGAGCGGCTTTATAAGACTCAGAAGGGAACAAACTTTTCAGTTGCGGTGGCTTCCCACAAGG
CACAGCGCTTTATTTGCATGCGCGCTAACCGGGGCCCCCTACTAAAAAGCCCGCGCATGTCCGGCGCGGGATGAT
GACGTCAGCCCCCGAGCGCG
>H1_352-H1_349 (SEQ ID NO: 1262)
GGGGAAGTGGGGGCAGGCCGGCTTTATAAGACTCCAGAGCGGAACGCATTTTTCAGTTATGGTGGCTTCCCACAA
GGCACAGCGCTATGCTTATTTCCATGGCCCCACCTCAGCATGGAAGCTCACGCCGCTTCTAGCCCGGGCCCCCTG
CTCCAGACAAAAAAGCCCGCGCATCCCGGCCGGGCGCGGGATGATGACGTCATCCCCAGCCCTCGAGCGCG
>H1_352-H1_354 (SEQ ID NO: 1263)
GGGAAGGCGGGGCCGGCGGCGCTAAAAGGCTCCGGGGCGGCCCGGACTTATCAGTTACGGTGGCTTCCCACGAGG
CGCAGCGCCGCTCATTTGCATGGCCCCACCCCAGACGGGAAGCCCGCGCCGCTCATTTGCGTGGCCCCGCCCCAG
ACGGGAAGCCCGCGCTGCTCGGCCGCGGTGGTGACGTCGGCCTCTCGCGCC
>H1_352-H1_356 (SEQ ID NO: 1264)
GGGAAAGCGGGGCCGGCGGCGCTAAAAGACTCCAGGGCGGCCCGGACTTATCAGTTACGGTGGCTTCCCACGAGG
CGCAGCGCCGCTCATTTGCATGGCCCCACCCCAGAAGGGAAGCCCGCGCCGCTCATTTGCGTGGCCCCGCCCCAG
ACGGGAAGCCCGCGCTGCCCGGCCGCGGTGGTGACGTCGGCCTCTCGCGCC
>H1_354-H1_355 (SEQ ID NO: 1265)
GGGAAGGCGGGGCCGGCGGCGCTAAAAGGCTCCGGGGCCGCCCGGACTTCACAGTTACGGTGGCTTCCCACGAGG
CGCAGCGCTGTCATTTGCATGGCCCCGCCCCAGACGGGAAGCCCGCGCTGCTCATTTGCGTGGCCCCGCCCCAGA
CGGGAAGCCCGCGCTGCTCGGCCGCGGTGGTGACGTCGGCCTCTCGCGCC
>H1_357-H1_358 (SEQ ID NO: 1266)
TGAAAGGGGCTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTATGACAGAGACCCTGCGATTCCTGGGAGCTGGCTGATGACGTCAGTGTCTAACCTCC
>H1_357-H1_359 (SEQ ID NO: 1267)
TGAAAGGAACTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTATGACAGAGACCCTGCGATTCCTGGGAGCTGGCTGATGACGTCAGTGTCTAACCTCC
>H1_357-H1_360 (SEQ ID NO: 1268)
TGAAAGGAACTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTGTGACAGAGACCCTGCGATTCCTGGGAGCTGGCTGATGACGTCAGTGTCTAACCTCC
>H1_357-H1_363 (SEQ ID NO: 1269)
TGAAAGGAACTCATCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTGTGACAGAGACCCTGCGATTCCTGGGAGCTGGCTGATGACGTCAGTGTCTAACCTCC
>H1_357-H1_365 (SEQ ID NO: 1270)
TGAGAGAAAATAAGCTCAAGCAGAACTTATAAGGCTCCCAAATGTACAGACATTTCTCGGTCATGGTAACTACCC
ACAACACACAGCGATATGCAAATATAGCAGAGTGTGCCTCCCCGCTCCCGTCCGGTCGTCTTCTCGCCGGAGCGC
AGGCGCGCTGCGTGGTGCGGGACTGTGACCCTGAGCCTGCGATTCCTGGGAGCGGGCTGATGACGTCAGCGTCTG
ACCTCC
>H1_357-H1_367 (SEQ ID NO: 1271)
TGAGAGAAACTAATCTCAAGCAGAACTTATAAGGCTCCCATATGTACAGACATTTCTCGGTCATGGTAACTACCC
ACAACACACAGCGATATGCAAATATAGCAGAGTGTGCCTCCCCGCTCGCGTCCGGTCGTCTTCTCGCCGGAGCGC
AGGCGCGCTGCGTGGTGCGGGACTGTGACCCTGAGCCTGCGATTCCTGGGAGCGGGCTGATGACGTCAGCGTCTA
ACCTCC
>H1_357-H1_368 (SEQ ID NO: 1272)
TGAGAGAAAGTAAGCTGAAGCAGAACTTATAAGGCTCCCAAATCTACAGACATTTCTCGGTCATGGTGACTACCC
ACAACACACAGCGATATGCAAATATCGCGGGGTGTGCCTCCCTGCTCTCGTCCGGTCGTCTTCTCGCCAGGGCGC
AGGCGCGCTGCGTGGTCCGGGCCTGTGACCCTGAGCCCGCGATTCCTGGGAGCGGGTTGATGACGTCAGCGTTTG
ACCTCC
>H1_357-H1_374 (SEQ ID NO: 1273)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCGCGGGGTGTGCGCCTCCCTGCTCTCGTCCAGTCGTCTTCTCGCCAGGGC
GCACGCGTACTAGCGCGCTGCGTTGTTCCCGGCCTGTGACAGAGCCTGAGCCCGCGATTTCCTGGGAGCGGGTTG
ATGACGTCAGCGTTTGAACTCC
>H1_357-H1_395 (SEQ ID NO: 1274)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCGCGGGGTGTGCGCCTCCCTGCTCTCGTCCAGTCGTCTTCTCGCCAGGGC
GCACGCGCGCTGCGTGTTCCCGGCCTGTGACCCTGAGCCCGCGATTCCTGGGAGCGGGTTGATGACGTCAGCGTT
TGAACTCC
>H1_363-H1_364 (SEQ ID NO: 1275)
TGAAAGGGACTCCTCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTCGGCGCCGGCGCGC
TGCGTGGGGCGGAACTGTGACAGAGACCCTGCGATTCCTGGGAGCTGGCTGATGACGTCAGTGTCTAACCTCC
>H1_364-H1_361 (SEQ ID NO: 1276)
TGAAAGGGACTCCTCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTCGGCGCCGGCGCGC
TGCGTGGGGCGGAACTGTGACAGAGACCCTGCGATTCCTGGGAGCTGGCTGATGACATCAGTGTCTAACCTCC
>H1_365-H1_366 (SEQ ID NO: 1277)
TGAGGGAAGATAAGCTCAAGCAGAACTTATAAGGCTCCCAAATGTACAGACATTTATCGGTCATGGTAACTACCC
ACAACACACAGCGATATGCAAATATAGCAGAGCGTGCCTCCTGCACGGGCCGGTCGTCTTCTCGCCGGAGCGCAG
GCGCGCTGCGTGGTGCGGGACTGTGACCCTGAGCCTGCGATTCCTGGGAGCGGGCTGATGACGTCAGCGTCTGAG
CTCC
>H1_369-H1_396 (SEQ ID NO: 1278)
TGGGAGAAAGTGGGCTGAAGCAGGACTTATAAGGCTCCCAAATCTAAAGACATTTTTTGGTCATGGTGACTTCCC
ACAACACACAGCGTCATGCAAATATCATGGGGTGTGCGCCTCCCTGCTCCCGTCCAGTCGTCTTCTCGCCAGGGC
GCACGCGCGCTGCGTGTTCCCGGCCTGTGACCCTGAGCCCGCGATTGCTGGGAGCGAGTTGATGACGTCAGCGTT
TGAACTCC
>H1_371-H1_372 (SEQ ID NO: 1279)
TGGGGAAAGCTGGGCTCAAGCAGAGCTTATAAGGCTCTCGTACCTAAAGACATTTCACGGTCATGGTGACTACCC
ACAACACACAGCGACATGCAAATTTCGTGGAGTGTGCCTCCCTCCGCTTGTCCCGCGTCTTTTCTCTCCCGGGCG
CACGCGCGCACGCACGCGACGCGTTCCCGCCACAGCGCCCCCGCGGTTCCTGGGAGCGGGTTGATGACGTCAGCA
TTTGGACGCC
>H1_374-H1_373 (SEQ ID NO: 1280)
TGAAAGAAACTAGCCACAAACGGAAACTATAAGAGGTCCAAAGCTCAGTGTACTCTATGGTTAGGGTGACTTCCC
ACAATACATAGCGATATGCAGATTTCTTCCCCAATCTGGCCCGCCGGGCCCTCCCTAGAGCGCATGCGCTGCAGG
TCCACGGCAGAGCACTGGGCGGGCGATCCCGGGAGCGGGTTGATGACGTCAGCGTTTGAACTCC
>H1_374-H1_375 (SEQ ID NO: 1281)
TGAAAGAAACTAGCCACAAACGGAAACTATAAGAGGTCCAAAGCTCAGTGTACTCTATGGTTAGGGTGACTTCCC
ACAATACATAGCGATATGCAGATTTCTTCCCCAGTCTGGCCCGCTGGGCCCTCCCTAGAGCGCATGCGCTGCAGG
TCCACGGCAGAGCACTGGGCGGGCGATCCCGGGAGCGGGTTGATGACGTCAGCGTTTGAACTCC
>H1_374-H1_376 (SEQ ID NO: 1282)
TGAAAGAAACTAGTTACAAACGGAAACTATAAGAGGTCCAAAGCTCAGTGTACTTTATGGTCAGGGTGACTTCCC
ACAATACATAGCGATATGTAGATTTCTTCCCCGATCTGGGCCCGCCGGGTCCTCCCTAGAGCGCATGCGCTGCAG
GTCCACGGCAGAGGACTGGGCGGGCGATTCCCGGGAGCGGGTTGATGACGTCAGCGTTTGAACTCC
>H1_374-H1_391 (SEQ ID NO: 1283)
TGAGAGAAAATGGTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTTCCTGGGAGCGAGTTGAT
GACGTCAGCGTTTGAACTCC
>H1_374-H1_392 (SEQ ID NO: 1284)
TGAAAGAAACTGGTTTCAAACGGAAACTATAAGAGGTCCAAATCTCAGTATACTTTTTGGTCAGGGTGACTTCCC
ACAATACACAGCGATATGTAGATTTCCTCCCCGATCTGGTCCCGTCGGCTCCTCGCTAGGGCGCATGCGCTGCAG
GTCCCCGGCCTATGACTGGGCCGGCGATTTCCCGGGAGCGAGTTGATGACGTCAGCGTTTGAACTCC
>H1_377-H1_378 (SEQ ID NO: 1285)
TGAAAAAAAAGGTTTCAAAGCTACACTTATAAGGCTCCCAAATGTCAGTATATTTTTTGGTCACGGTGACTTCCC
ACAATGCATAGCGATATGTAGATATTGCGAGGAGTACCTCCCAGTTCTGGTCCTGTCAGCTCTTTGCTAGGACGC
ACGCGCTGCAGGTTCCCAGCCTGTGATTGGGCCAGCGATTCCGGGAGCGAATTGATGACGTCAGCGTTTGAACTC
C
>H1_377-H1_380 (SEQ ID NO: 1286)
TGAAAAAAAAGGTTTCAAAGCTACACTTATAAGGCTCCCAAATCTCAGTATATTTTTTGGTCACGGTGACTTCCC
ACAATGCATAGCGATATGTAGATATTGCGAGGAGTACCTCCCAGTTCTGGTCCTGTCAGCTCTTTGCTAGGACGC
ACGCGCTGCAGGTTCCCAGCCTGTGATTGGGCCAGCGATTCCGGGAGCGAATTGATGACGTCAGCGTTTGAACTC
C
>H1_383-H1_377 (SEQ ID NO: 1287)
TGAAAGAAAAGGTTTCAAAGCTACACTTATAAGGATCCCAAATCTCAGTATATTTTTTGGTCACGGTGACTTCCC
ACAATACACAGCGATATGTAGATATCGCGAGGAGTACCTCCCAGTTCTGGTCCTGTCAGCTCTTTGCTAGGGCGC
ACGCGCTGCAGGTTCACAGCCTGTGATTGGGCCCGCGATTCCGGGAGCGAATTGATGACGTCAGCGTTTGAACTC
C
>H1_383-H1_384 (SEQ ID NO: 1288)
TGAAAGAAAAGGTTTCAAAGCTACACTTATAAGGATCCCAAATCTCAGTATATTTTTTGGTCACGGTGACTTCCC
ACAAGACACAGCGATATGTAGATATCGCGAGGAGTACCTCCCAGTTCTGGTCCTGTCAGCTCTTTGCTAGGGCGC
ACGCGCTGCAGGTTCACAGCCTGTGATTGGGCCCGCGATTCCGGGAGCGAATTGATGACGTCAGCGTTTGAACTC
C
>H1_386-H1_383 (SEQ ID NO: 1289)
TGAAAGAAAAAGTTTTGAAGCAGAACTTATAAGAATCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCGCTAGGGCGC
ACGCGCGCTGCATGGTTCACAGCCTGTGACCCTGGGCCCGCGATTCCTGGGAGCGAGTTGATGACGTCAGCGTTT
GAACTCC
>H1_386-H1_385 (SEQ ID NO: 1290)
TGAAAGCAAAAGTTTTGAAGCAGAACTTATAAGAAGCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCACTAGGGCGC
ATGCGCGCTGCATGGTTCACAGCCTGTGACCCTGGGCCTGCGATTCCTGGGAGCGAGTTGATGACGTCAGCGTTT
GAACTCC
>H1_386-H1_387 (SEQ ID NO: 1291)
TGAAAGCAAAAGTTTTGAAGCAGAACTTATAAGAAGCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCACTAGGGCGC
ATGCGCTGCAGGTTCACAGCCTGTGACTGGGCCTGCGATTCCTGGGAGCGAGTTGATGACGTCAGCGTTTGAACT
CC
>H1_388-H1_386 (SEQ ID NO: 1292)
TGAGAGAAAATGTTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTCCTGGGAGCGAGTTGATG
ACGTCAGCGTTTGAACTCC
>H1_388-H1_390 (SEQ ID NO: 1293)
TGAGAGAAAATGTTTTGAAGCAGAACTTATAAGAATCCCAAATCTCAGTATATTTTTTGGTCATGGTGACTTCCC
ACAATACACAGCGATATGTAGATATGGTGGGGAGCACCTCCCAGTTCTGGCCCAGTCGGCTCCTCGCTAGGGCGC
ACGCGTACTAGCGCGCTGCGGGTTCCCGGCCTGTGACAGTGCCTGAGCCCGCGATTCCTGGGAGCGAGTTGATGA
CGTCAGCGTTTGAACTCC
>H1_388-H1_393 (SEQ ID NO: 1294)
TAAGAGAAAGTTTTTTGAAGCAGAACTTATAAGGATCCCAAAACTCAGTATATTTTTTGGTCATGGTGACTTCCC
ACAATACACAGCGATATGTAGATATGGTGGGGAGCACCTCCCAGTTCTGGCCCAGTCGGCTCCTCGCTAGGGCGC
ACGCGTACTAGCGCGCTGCGGGTTCCCGGCCTGTGACAGTGCCTGAGCCCGCGATTCCTGGGAGCGAGTTGATGA
CGTCAGCGTTTGAACTCC
>H1_391-H1_388 (SEQ ID NO: 1295)
TGAGAGAAAATGGTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTCCTGGGAGCGAGTTGATG
ACGTCAGCGTTTGAACTCC
>H1_393-H1_394 (SEQ ID NO: 1296)
TAAGAGAAAGCTTTCTGAACCAGAGCTTATAAAGATCCCAAAACTCAGGCTATATTTTGGTCATGGTGACTTCCC
ACAATACACAGCGATATGTAGATATAGTGGGGAGCACCTCCCAGTTCTGGCCCAGTCGGGTCCTCTCTAGGGCGC
ACGCGCGCTGCGGGTTCCCGGCCTGTGACAGTGCCTGAGCCCGCGATTCCTGGGAGCGAGTTGACGTCACCGTTT
GAACTTC
>H1_395-H1_369 (SEQ ID NO: 1297)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCATGGGGTGTGCGCCTCCCTGCTCTCGTCCAGTCGTCTTCTCGCCAGGGC
GCACGCGCGCTGCGTGTTCCCGGCCTGTGACCCTGAGCCCGCGATTGCTGGGAGCGAGTTGATGACGTCAGCGTT
TGAACTCC
>H1_398-H1_357 (SEQ ID NO: 1298)
TGGGAAAAAGTGGGGCTCAAGCAGAATTTATAAGGCTCCCAAACCTAAAGACATTTTACGGTTATGGTGACTTCC
CACAACACACAGCGACATGCAAATATCGCGGGGTGTGCGGCCTCCCTGCTCTCGTCCAGGCGTCTTCTCGCCAGG
GCGCACGCGCGCACGCGCGCTGCGCTGTTCCCGCCCTGGTGACGGAGCCTGAGCCCGCGATTTCCTGGGAGCGGG
TTGATGACGTCAGCGTTTGGACTCC
>H1_398-H1_399 (SEQ ID NO: 1299)
CAGGAAAGACTGCGCTGAGGCAGACTTTATAAGGCTCCCGCGCAGAAAGAAACTTTATAGTTATGGTGATTTCCC
ACAAGCCACTGCGTCATGCAAATAAAGCAGGGTTGACGGCTTCCAAGTATGTACCTTAAGGTTTTTCTCTAGGCC
GCGTACGCTCTGCGTATTCAGCCACGTGACCCTGAGCCAGTGGTTGTTGGGAGCACGTTGTGGACCTCTGCGTTT
GGATTCC
>H1_398-H1_400 (SEQ ID NO: 1300)
CAGGAAAGAGTGGGGCTCAGGCAGACTTTATAAGGCTCCCAAACAGAAAGACACTTTACAGTTATGGTGACTTCC
CACAAGACACTGCGTCATGCAAATATCGCAGGGTTGGCGGCCTTCCTTCTATCTTCCTTAAGGTTTCTCTCTAGG
GCGCGTACGCGCTGCGTATTCCCGCCCCGGTGACCCTGAGCCAGTGGTTGTTGGGAGCACGTTGATGACGTCTGC
GTTTGGATTCC
>H1_402-H1_403 (SEQ ID NO: 1301)
TGGGGAGTGGCCGCCTAGGGGGCGATATATAAGGCTCACAAAACCCGTGCTATTTCTTACAGAGGGTGAATATCC
CCATGATCCTCGGCGGCATGCAAATAATAGTTGCGTCAGAGTAGAGCGCAGCCTGCCGGTCTCTCCTAGCGCGGG
AAATCCTGTTTTCTTCTTCAGTCCCGGTGACGAGGACGCGCGCGCGCACCGTAGCCGGACAACGGTCTGGTAAGG
TAGGCGGGATTCGGTTGAGAGCGCC
>H1_403-H1_404 (SEQ ID NO: 1302)
CGTGGAATCCCCGCCTAGGGGGCGCTATATAAGGCTCACCAAACCCGTGCTATTTCTTACAGAGGGTGAATATCC
CATGATCCTTGGCGGCATGCAAATAACAGCTTGCGTCAGAGTAGAGCGCAGCCTACCAGTCTTTCCTAGCGCGGG
AAATCCCGTTTTCTTCTGAGGTCGCCGGTGACGCGCGCGTGCGCCGTAGCCAGAGAACGGTCCGGGAAGGTAGGC
CGGCCGGGATTCGGTTGAGAGCGCC
>H1_407-H1_408 (SEQ ID NO: 1303)
TGGGACAAAAAACTCTTGGTCACATTATATAAGAATCCCATATCTAAAGACATTTCAGGGTTAGGGTGACTTCCC
CAACAATACATAGCGACATGCAAATATCATGGTCCTTCCAGGAGGCGTGCCTCCCCGTCCCCTTGGTCCAGGTCT
TGCTGGGGCGCACGCGCGCTGCGTGTTCCCGCTCTGTGACTCTCAGCTCGCGATTCCTGAGAGCGGATTGGTGAA
GTCAATGTTCTGGCTCC
>FIG. 17 Consensus Sequence (SEQ ID NO: 1868)
TGAGCTTCCCTCCGCCCTATGRGRAARRGTGGTYCYAYNCAGAACTTATAAGRYTCCCAWAYYYAAAGACATTTC
WCGWTTATGGTGAYTTCCCAGAABACAYAGCGACATGCAAATATTGYAGGGCGTSMCWCCCCTGTCCCTNACRGY
CRTCTTCCTGCCAGGGCGCACGCGCGCTGSGTGTTCCCGCSTAGTGACDCTGGGCCCGCGATTCCTTGGAGCGGG
TTGATGACGTCAGCGTTCGAATTCCATGGCG

What is claimed is:

1. A nucleic acid comprising a compact promoter operably linked to a coding sequence of a large gene, wherein the compact promoter is between 50 and 250 bp, and wherein the coding sequence of the large gene is greater than about 4110 bp.

2. The nucleic acid of claim 1, wherein the compact promoter is between 50 and 225 bp.

3. The nucleic acid of claim 1, wherein the compact promoter is between 50 and 200 bp.

4. The nucleic acid of claim 1, wherein the compact promoter is between 50 and 180 bp.

5. The nucleic acid of any preceding claim, wherein the compact promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303 or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

6. The nucleic acid of any preceding claim, wherein the compact promoter comprises an H1 promoter.

7. The nucleic acid of claim 6, wherein the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303 or any sequence in FIGS. 3-19 that corresponds to the H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

8. The nucleic acid of claim 6 or 7, wherein the H1 promoter comprises a human H1 promoter.

9. The nucleic acid of any one of claims 1-5, wherein the compact promoter comprises a Gar1 promoter.

10. The nucleic acid of claim 9, wherein the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto.

11. The nucleic acid of claim 9 or 10, wherein the Gar1 promoter is a human Gar1 promoter.

12. The nucleic acid of any one of claims 1-5, wherein the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

13. The nucleic acid of any preceding claim, wherein the compact promoter does not comprise a viral promoter and/or a synthetic promoter.

14. The nucleic acid of any preceding claim, wherein the compact promoter does not comprise F5tg83.

15. The nucleic acid of any preceding claim, wherein the compact promoter comprises at least 95%, 98%, 99%, 99.5% or 100% identity to a naturally-occurring mammalian promoter.

16. The nucleic acid of any preceding claim, wherein the compact promoter is capable of expressing a luciferase reporter at a higher level than is a HSK thymidine kinase (TK) promoter.

17. The nucleic acid of any preceding claim, wherein the coding sequence encodes a cystic fibrosis transmembrane conductance regulator (CFTR), ATP7B, ATP7A, AGL, CPS1, or a functional fragment or variant thereof.

18. The nucleic acid of any preceding claim, wherein the coding sequence encodes a cystic fibrosis transmembrane conductance regulator (CFTR).

19. The nucleic acid of claim 17 or 18, wherein the CFTR coding sequence is codon optimized.

20. The nucleic acid of claim 19, wherein the codon-optimized CFTR coding sequence comprises one or more of the following features as compared to a wild type CFTR coding sequence:
(a) fewer unpaired base pairs of mRNA;
(b) increased codon usage bias;
(c) decreased GC content;
(d) fewer CpG dinucleotides;
(e) increased mRNA secondary structure;
(f) fewer cryptic splicing sites;
(g) fewer premature poly(A) sites;
(h) fewer RNA instability motifs;
(i) fewer AT-rich elements (ARE);
(j) fewer repeat sequences (e.g., direct repeat, reverse repeat, and dyad repeat);
(k) fewer GC peaks; and
(l) fewer cis-acting elements.

21. The nucleic acid of any preceding claim, wherein the CFTR coding sequence comprises a truncated form of a wild-type CFTR gene.

22. The nucleic acid of claim 21, wherein the truncated form of the wild-type CFTR gene comprises CTFRΔR.

23. An expression construct comprising the nucleic acid of any preceding claim.

24. The expression construct of claim 23, wherein the coding sequence can be expressed in a target cell.

25. The expression construct of claim 24, wherein the target cell is a lung cell, a pancreatic cell, a liver cell, or a neuronal cell.

26. The expression construct of claim 23 or 24, wherein the expression construct can be expressed in Calu-3, CFBE4lo−, or A549 cells.

27. The expression construct of claim 23 or 24, wherein the expression construct can be expressed in HEK293 cells.

28. The expression construct of claim 23 or 24, wherein the expression construct can be expressed in HeLa cells.

29. The expression construct of claim 23 or 24, wherein the coding sequence encodes a CFTR, and, when the expression construct is expressed in an epithelial cell, the expressed CFTR protein causes an increase in transepithelial electrical resistance as compared to a cell in which the expression construct is not present.

30. The expression construct of any one of claims 23, 24, and 29, wherein the coding sequence encodes a CFTR, and, when the expression construct is expressed in an epithelial cell, the expressed CFTR protein causes an increase in transepithelial Cl⁻ transport as compared to a cell in which the expression construct is not present.

31. A vector comprising the expression construct of any one of claims 23-30.

32. The vector of claim 31, wherein the vector comprises an adeno-associated viral (AAV) vector.

33. The vector of claim 32, wherein the AAV vector comprises an AAV-6 vector.

34. A method of expressing a protein in a cell, the method comprising transfecting a cell with the expression construct of any one of claims 23-30 or the vector of any one of claims 31-33.

35. A method of treating a disease (e.g., cystic fibrosis, Wilson disease, Menkes disease, Cori Disease, or carbamoyl phosphate synthetase I deficiency (CPS1D)) in a subject in need thereof, the method comprising administering to the subject a vector of any one of claims 31-33.

36. A nucleic acid comprising a cystic fibrosis transmembrane conductance regulator (CFTR) coding sequence, wherein the CFTR coding sequence is codon-optimized, wherein the CFTR coding sequence comprises one or more of the following features as compared to a wild type CFTR coding sequence:
(a) fewer unpaired base pairs of mRNA;
(b) increased codon usage bias;
(c) decreased GC content;
(d) fewer CpG dinucleotides;
(e) increased mRNA secondary structure;
(f) fewer cryptic splicing sites;
(g) fewer premature poly(A) sites;
(h) fewer RNA instability motifs;
(i) fewer AT-rich elements (ARE);
(j) fewer repeat sequences (e.g., direct repeat, reverse repeat, and dyad repeat);
(k) fewer GC peaks; and
(l) fewer cis-acting elements.

37. The nucleic acid of claim 20 or claim 36, wherein the codon usage bias is determined using the codon adaptive index (CAI).

38. The nucleic acid of claim 37, wherein the CAI score is greater than about 0.70.

39. The nucleic acid of any one of claims 20 and 36-38, wherein the frequency of optimal codons (FOP) is greater than about 80%.

40. The nucleic acid of any one of claims 20 and 36-39, wherein the cis-acting element is selected from the group consisting of splice donors/acceptors (e.g., GGTAAG, GGTGAT, GTAAAA, GTAAGT), PolyA (e.g., AATAAA, ATTAAA, AAAAAAA), destabilizing motifs (e.g., ATTTA), AT-rich elements (e.g., ATTTTA, ATTTTTA, ATTTTTTA), PolyT, polymerase slippage sites (e.g., GGGGGG, CCCCCC), and internal Kozak sequences (e.g., ACCACCATGG, GCCACCATGG).

41. The nucleic acid of any one of claims 20 and 36-40, wherein the nucleic acid further comprises a 3′UTR, a 5′UTR or a 3′UTR and a 5′UTR.

42. The nucleic acid of claim 41, wherein the minimum free energy structure of the nucleic acid comprising the 3′UTR, the 5′UTR or the 3′UTR and the 5′UTR does not favor base-pairing between (a) the 3′UTR, the 5′UTR or the 3′UTR and the 5′UTR and (b) the CFTR coding sequence.

43. An expression construct comprising the nucleic acid of any one of claims 36-42.

44. The expression construct of claim 43, wherein the half-life of the mRNA expressed from the codon optimized CFTR coding sequence is increased as compared to a wild-type CFTR coding sequence.

45. The expression construct of any one of claims 43-44, wherein expression of the codon optimized CFTR coding sequence results in an increased amount of CFTR mRNA or protein as compared to expression of a wild-type CFTR coding sequence.

46. The expression construct of any one of claims 43-45, wherein the CFTR coding sequence can be expressed in the lung and/or the pancreas.

47. The expression construct of any one of claims 43-46, wherein the expression construct can be expressed in HEK293 or A549 cells.

48. The expression construct of any one of claims 43-46, wherein, when the expression construct is expressed in an epithelial cell, the expressed CFTR protein causes an increase in transepithelial electrical resistance as compared to a cell in which the expression construct is not present.

49. The expression construct of any one of claims 43-46 and 48, wherein, when the expression construct is expressed in an epithelial cell, the expressed CFTR protein causes an increase in transepithelial Cl⁻ transport as compared to a cell in which the expression construct is not present.

50. A vector comprising the expression construct of any one of claims 43-49.

51. The vector of claim 50, wherein the vector comprises an adeno-associated viral (AAV) vector.

52. The vector of claim 51, wherein the AAV vector comprises an AAV-6 vector.

53. A method of expressing a CFTR protein in a cell, the method comprising transfecting a cell with the expression construct of any one of claims 43-49 or the vector of any one of claims 50-52.

54. A method of treating cystic fibrosis in a subject in need thereof, the method comprising administering to the subject a vector of any one of claims 46-49.

55. A nucleic acid comprising:
a) a compact bidirectional promoter;
b) a protein coding gene; and
c) a second gene;
wherein the compact bidirectional promoter has a size between 50 and 250 bp, comprises at least one regulatory element that provides for transcription of the protein coding gene in one direction and at least one regulatory element that provides for transcription of the second gene in the other direction, wherein the protein coding gene comprises a coding sequence from about 300 bp to about 4110 bp.

56. The nucleic acid of claim 55, wherein the compact bidirectional promoter is between 50 and 225 bp.

57. The nucleic acid of claim 55, wherein the compact bidirectional promoter is between 50 and 200 bp.

58. The nucleic acid of claim 55, wherein the compact bidirectional promoter is between 50 and 180 bp.

59. The nucleic acid of any one of claims 55-58, wherein the second gene encodes an RNA or a second protein.

60. The nucleic acid of any one of claims 55-60, wherein the compact bidirectional promoter comprises an H1 promoter.

61. The nucleic acid of claim 60, wherein the H1 promoter comprises a human H1 promoter.

62. The nucleic acid of any one of claims 55-61, wherein the coding sequence encodes a cystic fibrosis transmembrane conductance regulator (CFTR), ATP7B, ATP7A, AGL, CPS1, or a functional fragment or variant thereof.

63. The nucleic acid of any one of claims 55-61, wherein the coding sequence encodes a cystic fibrosis transmembrane conductance regulator (CFTR).

64. The nucleic acid of claim 62 or 63, wherein the CFTR coding sequence is codon optimized.

65. The nucleic acid of claim 64, wherein the codon-optimized CFTR coding sequence comprises one or more of the following features as compared to a wild type CFTR coding sequence:
(a) fewer unpaired base pairs of mRNA;
(b) increased codon usage bias;
(c) decreased GC content;
(d) fewer CpG dinucleotides;
(e) increased mRNA secondary structure;
(f) fewer cryptic splicing sites;
(g) fewer premature poly(A) sites;
(h) fewer RNA instability motifs;
(i) fewer AT-rich elements (ARE);
(j) fewer repeat sequences (e.g., direct repeat, reverse repeat, and dyad repeat);
(k) fewer GC peaks; and
(l) fewer cis-acting elements.

66. The nucleic acid of any one of claims 62-65, wherein the CFTR coding sequence comprises a truncated form of a wild-type CFTR gene.

67. The nucleic acid of claim 66, wherein the truncated form of the wild-type CFTR gene comprises CTFRΔR.

68. An expression construct comprising the nucleic acid of any one of claims 55-67.

69. The expression construct of claim 68, wherein the coding sequence can be expressed in a target cell.

70. The expression construct of claim 69, wherein the target cell is a lung cell, a pancreatic cell, a liver cell, or a neuronal cell.

71. The expression construct of claim 68 or 69, wherein the expression construct can be expressed in Calu-3, CFBE4lo−, or A549 cells.

72. The expression construct of claim 68 or 69, wherein the expression construct can be expressed in HEK293 cells.

73. The expression construct of claim 68 or 69, wherein the expression construct can be expressed in HeLa cells.

74. The expression construct of claim 68 or 69, wherein the coding sequence encodes a CFTR, and, when the expression construct is expressed in an epithelial cell, the expressed CFTR protein causes an increase in transepithelial electrical resistance as compared to a cell in which the expression construct is not present.

75. The expression construct of any one of claim 68, 69, and 74, wherein the coding sequence encodes a CFTR, and, when the expression construct is expressed in an epithelial cell, the expressed CFTR protein causes an increase in transepithelial Cl⁻ transport as compared to a cell in which the expression construct is not present.

76. A vector comprising the expression construct of any one of claims 68-75.

77. The vector of claim 76, wherein the vector comprises an adeno-associated viral (AAV) vector.

78. The vector of claim 77, wherein the AAV vector comprises an AAV-6 vector.
